(19) United States
(12) Patent Application Publication
(10) Pub. No.: US 2026/0176603 A1
(43) Pub. Date: Jun. 25, 2026

(54) TYPE II CAS PROTEINS AND APPLICATIONS THEREOF

(71) Applicant: Alia Therapeutics Srl, Milano (IT)

(72) Inventors: Antonio CASINI, Trento (IT); Laura PEZZÈ, Trento (IT); Matteo CICIANI, Schio (IT)

(73) Assignee: Alia Therapeutics Srl, Milano (IT)

(21) Appl. No.: 19/129,627

(22) PCT Filed: Nov. 16, 2023
(86) PCT No.: PCT/EP2023/082056

§ 371 Date: May 13, 2025

Related U.S. Application Data
(60) Provisional application No. 63/425,874, filed on Nov. 16, 2022.
(60) Provisional application No. 63/481,616, filed on Jan. 26, 2023.

(51) Int. Cl. C12N 9/22; C12N 15/11; C12N 15/113

(52) U.S. Cl. CPC C12N 9/22; C12N 15/11; C12N 15/111; C12N 15/113; C12N 2310/20

(57) ABSTRACT

Type II Cas proteins, for example Type II Cas proteins referred to as AHZW, ABSE, AIXM, AXTQ, AIWM, AIWR, AIYQ, EQSC, BDLP, and BDKL Type II Cas proteins; gRNAs for Type II Cas proteins; systems comprising Type II Cas proteins and gRNAs; nucleic acids encoding the Type II Cas proteins, gRNAs and systems; particles comprising the foregoing; pharmaceutical compositions of the foregoing; and uses of the foregoing, for example to alter the genomic DNA of a cell.

### 1. CROSS-REFERENCE TO RELATED APPLICATIONS

This application claims the priority benefit of U.S. provisional application Nos. 63/425,874, filed Nov. 16, 2022, and 63/481,616, filed Jan. 26, 2023, the contents of which are incorporated herein in their entireties by reference thereto.

### 2. SEQUENCE LISTING

The instant application contains a Sequence Listing which has been submitted electronically in XML format and is hereby incorporated by reference in its entirety. Said XML Sequence Listing, created on Nov. 13, 2023, is named ALA-010WO_SL.xml and is 534,992 bytes in size.

### 3. BACKGROUND

CRISPR-Cas genome editing with Type II Cas proteins and associated guide RNAs (gRNAs) is a powerful tool with the potential to treat a variety of genetic diseases. Adeno-associated viral vectors (AAVs) are commonly used to deliver Cas proteins, for example Streptococcus pyogenes Cas9 (SpCas9), and their guide RNAs (gRNAs). However, packaging a large Cas protein such as SpCas9 together with a guide RNA into a single AAV vector can be challenging due to the limited packaging capacity of AAVs. Thus, there is a need for Type II Cas nucleases with smaller sizes that can be packaged together with a gRNA in a single AAV. In addition, the discovery of novel nucleases with new PAM specificities can broaden the range of targetable sites in the cell genome, making genome editing more flexible and efficient.

### 4. SUMMARY

This disclosure is based, in part, on the discovery of a Type II Cas protein from an unclassified bacterium from the Solobacterium genus (referred to herein as “wild-type AHZW Type II Cas”), a Type II Cas protein from an unclassified bacterium from the Acholeplasmatales order (referred to herein as “wild-type ABSE Type II Cas”), a Type II Cas protein from an unclassified bacterium from the Bacilli class (referred to herein as “wild-type AIXM Type II Cas”), a Type II Cas protein from an unclassified bacterium from the Lachnospiraceae family (referred to herein as “wild-type AXTQ Type II Cas”), a Type II Cas protein from an unclassified bacterium from the Bacilli class (referred to herein as “wild-type AIWM Type II Cas”), a Type II Cas protein from an unclassified bacterium from the Bacilli class (referred to herein as “wild-type AIWR Type II Cas”), a Type II Cas protein from an unclassified bacterium from the Bacilli class (referred to herein as “wild-type AIYQ Type II Cas”), a Type II Cas protein from Slackia faecicanis (referred to herein as “wild-type EQSC Type II Cas”), a Type II Cas protein from unclassified bacterium from the Slackia genus (referred to herein as “wild-type BDLP Type II Cas”), and a Type II Cas protein from an unclassified bacterium from the Atopobiaceae family (referred to herein as “wild-type BDKL Type II Cas”). Wild-type AHZW, ABSE, AIXM, AXTQ, AIWM, AIWR, AIYQ, EQSC, BDLP, and BDKL Type II Cas proteins are each approximately 1000 amino acids in length, significantly shorter than SpCas9.

In one aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) to SEQ ID NO:1 (such proteins referred to herein as “AHZW Type II Cas proteins”). Exemplary AHZW Type II Cas protein sequences are set forth in SEQ ID NO:1, SEQ ID NO:2, and SEQ ID NO:3.

In one aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) to SEQ ID NO:7 (such proteins referred to herein as “ABSE Type II Cas proteins”). Exemplary ABSE Type II Cas protein sequences are set forth in SEQ ID NO:7, SEQ ID NO:8, and SEQ ID NO:9.

In one aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) to SEQ ID NO:13 (such proteins referred to herein as “AIXM Type II Cas proteins”). Exemplary AIXM Type II Cas protein sequences are set forth in SEQ ID NO:13, SEQ ID NO:14, and SEQ ID NO:15.

In one aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) to SEQ ID NO:19 (such proteins referred to herein as “AXTQ Type II Cas proteins”). Exemplary AXTQ Type II Cas protein sequences are set forth in SEQ ID NO:19, SEQ ID NO:20, and SEQ ID NO:21.

In one aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) to SEQ ID NO:25 (such proteins referred to herein as “AIWM Type II Cas proteins”). Exemplary AIWM Type II Cas protein sequences are set forth in SEQ ID NO:25, SEQ ID NO:26 and SEQ ID NO:27.

In one aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) to SEQ ID NO:31 (such proteins referred to herein as “AIWR Type II Cas proteins”). Exemplary AIWR Type II Cas protein sequences are set forth in SEQ ID NO:31, SEQ ID NO:32 and SEQ ID NO:33.

In one aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) to SEQ ID NO:37 (such proteins referred to herein as “AIYQ Type II Cas proteins”). Exemplary AIYQ Type II Cas protein sequences are set forth in SEQ ID NO:37, SEQ ID NO:38 and SEQ ID NO:39.

In one aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) to SEQ ID NO:43 (such proteins referred to herein as “EQSC Type II Cas proteins”). Exemplary EQSC Type II Cas protein sequences are set forth in SEQ ID NO:43, SEQ ID NO:44, and SEQ ID NO:45.

In one aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) to SEQ ID NO:49 (such proteins referred to herein as “BDLP Type II Cas proteins”). Exemplary BDLP Type II Cas protein sequences are set forth in SEQ ID NO:49, SEQ ID NO:50, and SEQ ID NO:51.

In one aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) to SEQ ID NO:55 (such proteins referred to herein as “BDKL Type II Cas proteins”). Exemplary BDKL Type II Cas protein sequences are set forth in SEQ ID NO:55, SEQ ID NO:56, and SEQ ID NO:57.

In another aspect, the disclosure provides Type II Cas proteins comprising an amino acid sequence having at least 50% (e.g., at least 55%, at least 60%, at least 65%, at least 70%, at least 75%, at least 85%, at least 90%, at least 95%, or more) sequence identity to a RuvC-I domain, RuvC-II domain, RuvC-III domain, BH domain, REC domain, HNH domain, WED domain, or PID domain of a AHZW Type II Cas protein, ABSE Type II Cas protein, AIXM Type II Cas protein, AXTQ Type II Cas protein, AIWM Type II Cas protein, AIWR Type II Cas protein, AIYQ Type II Cas protein, EQSC Type II Cas protein, BDLP Type II Cas protein, or a BDKL Type II Cas protein. In some embodiments, a Type II Cas protein of the disclosure is a chimeric Type II Cas protein, for example, comprising one or more domains from an AHZW, ABSE, AIXM, AXTQ, AIWM, AIWR, AIYQ, EQSC, BDLP, and/or BDKL Type II Cas protein(s) and one or more domains from a different Type II Cas protein such as SpCas9.

In some embodiments, the Type II Cas proteins of the disclosure are in the form of a fusion protein, for example, comprising an AHZW Type II Cas protein, ABSE Type II Cas protein, AIXM Type II Cas protein, AXTQ Type II Cas protein, AIWM Type II Cas protein, AIWR Type II Cas protein, AIYQ Type II Cas protein, EQSC Type II Cas protein, BDLP Type II Cas protein, or a BDKL Type II Cas protein sequence fused to one or more additional amino acid sequences, for example, one or more nuclear localization signals and/or one or more tags. Other exemplary fusion partners can enable base editing (e.g., where the fusion partner is nucleoside deaminase) or prime editing (e.g., where the fusion partner is a reverse transcriptase).

Exemplary features of Type II Cas proteins of the disclosure are described in Section 6.2 and specific embodiments 1 to 255 and 756 to 762, infra.

In further aspects, the disclosure provides guide (gRNA) molecules, for example single guide RNAs (sgRNAs), and combinations of two or more gRNA molecules (e.g., combinations of sgRNA molecules). In various embodiments, the disclosure provides gRNAs that can be used with the AHZW Type II Cas proteins of the disclosure, gRNAs that can be used with the ABSE Type II Cas proteins of the disclosure, gRNAs that can be used with the AIXM Type II Cas proteins of the disclosure, gRNAs that can be used with the AXTQ Type II Cas proteins of the disclosure, gRNAs that can be used with the AIWM Type II Cas proteins of the disclosure, gRNAs that can be used with the AIWR Type II Cas proteins of the disclosure, gRNAs that can be used with the AIYQ Type II Cas proteins of the disclosure, gRNAs that can be used with the EQSC Type II Cas proteins of the disclosure, gRNAs that can be used with the BDLP Type II Cas proteins of the disclosure, and gRNAs that can be used with the BDKL Type II Cas proteins of the disclosure. Exemplary features of the gRNAs of the disclosure and combinations of gRNAs are described in Section 6.3 and specific embodiments 256 to 611, infra.

In further aspects, the disclosure provides systems comprising a Type II Cas protein of the disclosure and one or more gRNAs, e.g., sgRNAs. For example, a system can comprise a ribonucleoprotein (RNP) comprising a Type II Cas protein complexed with a gRNA, e.g., an sgRNA or separate crRNA and tracrRNA. Exemplary features of systems are described in Section 6.4 and specific embodiments 614 to 698, infra.

In another aspect, the disclosure provides nucleic acids and pluralities of nucleic acids encoding a Type II Cas protein of the disclosure and, optionally, a guide RNA, for example a sgRNA. In some embodiments, the nucleic acids comprise a Type II Cas protein of the disclosure operably linked to a heterologous promoter, e.g., a mammalian promoter, for example a human promoter.

In another aspect, the disclosure provides nucleic acids encoding a gRNA, for example a sgRNA, of the disclosure and, optionally, a Type II Cas protein, for example an AHZW Type II Cas protein, ABSE Type II Cas protein, AIXM Type II Cas protein, AXTQ Type II Cas protein, AIWM Type II Cas protein, AIWR Type II Cas protein, AIYQ Type II Cas protein, EQSC Type II Cas protein, BDLP Type II Cas protein, or a BDKL Type II Cas protein.

In another aspect, the disclosure provides nucleic acids encoding combinations of gRNAs of the disclosure, for example a combination of two gRNAs, and, optionally, a Type II Cas protein.

Exemplary features of nucleic and pluralities of nucleic acids of the disclosure are described in Section 6.5 and specific embodiments 699 to 755, infra.

In further aspects, the disclosure provides particles comprising the Type II Cas proteins, gRNAs, nucleic acids, and systems of the disclosure. Exemplary features of particles of the disclosure are described in Section 6.6 and specific embodiments 763 to 778, infra.

In another aspect, the disclosure provides cells and populations of cells containing or contacted with a Type II Cas protein, gRNA, nucleic acid, plurality of nucleic acids, system, or particle of the disclosure. Exemplary features of such cells and cell populations are described in Section 6.6 and specific embodiments 780 to 787 and 824, infra.

In another aspect, the disclosure provides pharmaceutical compositions comprising a Type II Cas protein, gRNA, nucleic acid, plurality of nucleic acids, system, particle, cell, or population of cells together with one or more excipients. Exemplary features of pharmaceutical compositions are described in Section 6.7 and specific embodiment 779, infra.

In another aspect, the disclosure provides methods of altering cells (e.g., editing the genome of a cell) using the Type II Cas proteins, gRNAs, nucleic acids, systems, particles, and pharmaceutical compositions of the disclosure. Cells altered according to the methods of the disclosure can be used, for example, to treat subjects having a disease or disorder, e.g., genetic disease or disorder, for example retinitis pigmentosa caused by a RHO mutation. Features of exemplary methods of altering cells are described in Section 6.8 and specific embodiments 788 to 823, infra.

### 5. BRIEF DESCRIPTION OF THE FIGURES

FIGS. 1A-1G show predicted PAM logos for exemplary Type IIA Cas proteins of the disclosure: AHZW (FIG. 1A), ABSE (FIG. 1B), AIWM (FIG. 1C), AIWR (FIG. 1D), AIXM (FIG. 1E), AIYQ (FIG. 1F), and AXTQ (FIG. 1G).

FIG. 2A-2D show exemplary Type II Cas protein sgRNAs. Schematic representation of the hairpin structure generated for visualization after in silico folding using RNA folding form v2.3 (www.unafold.org) of the sgRNA scaffolds (not including the spacer sequence) designed from crRNAs and tracrRNAs identified for ABSE Type II Cas protein (FIG. 2A), AHZW Type II Cas protein (FIG. 2B), AIWM-AIWR-AIYQ Type II Cas proteins (FIG. 2C) and AIXM Type II Cas protein (FIG. 2D). These sgRNAs have been trimmed by the insertion of a GAAA tetraloop at the level of the repeat:antirepeat loop to contain their size. AIWM, AIWR and AIYQ Type II Cas proteins can share the same sgRNA scaffold. FIG. 2A discloses SEQ ID NO: 96, FIG. 2B discloses SEQ ID NO: 94, FIG. 2C discloses SEQ ID NO: 102, and FIG. 2D discloses SEQ ID NO: 98.

FIGS. 3A-3D show exemplary Type II Cas protein sgRNAs. Schematic representation of the hairpin structure generated for visualization after in silico folding using RNA folding form v2.3 (www.unafold.org) of the sgRNA scaffolds (not including the spacer sequence) designed from crRNAs and tracrRNAs identified for AXTQ Type II Cas protein (FIG. 3A), BDKL Type II Cas protein (FIG. 3B), BDLP Type II Cas proteins (FIG. 3C) and EQSC Type II Cas protein (FIG. 3D). These sgRNAs have been trimmed by the insertion of a GAAA tetraloop at the level of the repeat:antirepeat loop to contain their size. FIG. 3A discloses SEQ ID NO: 100, FIG. 3B discloses SEQ ID NO: 108, FIG. 3C discloses SEQ ID NO: 106, and FIG. 3D discloses SEQ ID NO: 104.

FIGS. 4A-4F show AIWM, AIWR and ABSE Type II Cas protein PAM specificities (Example 2). FIGS. 4A, 4C, and 4E show PAM sequence logos for AIWM Type II Cas (FIG. 4A), AIWR Type II Cas (FIG. 4C) and ABSE Type II Cas (FIG. 4E) resulting from the in vitro PAM assay. FIGS. 4B, 4D, and 4F show PAM enrichment heatmaps calculated from the same in vitro PAM assay showing the nucleotide preferences at different positions along the PAM for AIWM Type II Cas (FIG. 4B, positions 2, 3 and 4, 5), AIWR Type II Cas (FIG. 4D, left panel: positions 2, 3 and 4, 5; right panel: positions 3, 4 and 5, 7), ABSE Type II Cas (FIG. 4F, positions 2, 3 and 4, 5).

FIGS. 5A-5F show AIXM, AHZW and AIYQ Type II Cas protein PAM specificities (Example 2). FIGS. 5A, 5C, and 5E show PAM sequence logos for AIXM Type II Cas (FIG. 5A), AHZW Type II Cas (FIG. 5C) and AIYQ Type II Cas (FIG. 5E) resulting from the in vitro PAM assay. FIGS. 5B, 5D, and 5F show PAM enrichment heatmaps calculated from the same in vitro PAM assay showing the nucleotide preferences at different positions along the PAM for AIXM Type II Cas (FIG. 5B, positions 2, 3 and 4, 5), AHZW Type II Cas (FIG. 5D, left panel: positions 2, 3 and 4, 5; right panel: positions 2, 4 and 5, 7), AIYQ Type II Cas (FIG. 5F, positions 3, 4 and 5, 6).

FIGS. 6A-6F show BDLP, BDKL and EQSC Type II Cas protein PAM specificities (Example 2). FIGS. 6A, 6C, and 6E show PAM sequence logos for BDLP Type II Cas (FIG. 6A), BDKL Type II Cas (FIG. 6C) and EQSC Type II Cas (FIG. 6E) resulting from the in vitro PAM assay. FIGS. 6B, 6D, and 6F show PAM enrichment heatmaps calculated from the same in vitro PAM assay showing the nucleotide preferences at different positions along the PAM for BDLP Type II Cas (FIG. 6B, positions 3, 5 and 6, 7), BDKL Type II Cas (FIG. 6D, positions 5, 6 and 7, 8), EQSC Type II Cas (FIG. 6F, positions 5, 6 and 7, 8).

FIGS. 7A-7B show AXTQ Type II Cas protein PAM specificities (Example 2). FIG. 7A shows a PAM sequence logo for AXTQ Type II Cas resulting from the in vitro PAM assay. FIG. 7B shows PAM enrichment heatmaps calculated from the same in vitro PAM assay showing the nucleotide preferences at different positions along the PAM for AXTQ Type II Cas (left panel: positions 3, 4 and 7, 8; right panel: positions 4, 5 and positions 6, 8).

FIG. 8 shows an exemplary full-length sgRNA scaffold for AIWM, AIWR and AIYQ Type II Cas proteins. Schematic representation of the hairpin structure generated for visualization after in silico folding using RNA folding form v2.3 (www.unafold.org) of the full-length sgRNA scaffold (not including the spacer sequence) designed by direct fusion with a GAAA tetraloop with minimal trimming of the crRNA and tracrRNA identified for AIWM-AIWR-AIYQ Type II Cas proteins, which can share the same sgRNA scaffold. FIG. 8 discloses SEQ ID NO: 101.

FIGS. 9A-9C show AIWM and AIWR Type II Cas protein PAM specificities using a full-length sgRNA scaffold (Example 2). FIGS. 9A and 9C show PAM sequence logos for AIWM Type II Cas (FIG. 9A) and AIWR Type II Cas (FIG. 9C) resulting from the in vitro PAM assay performed using a full-length in vitro transcribed sgRNA scaffold. FIGS. 9B and 9D show PAM enrichment heatmaps calculated from the same in vitro PAM assay showing the nucleotide preferences at different positions along the PAM for AIWM Type II Cas (FIG. 9B, positions 2, 3 and 4, 5), AIWR Type II Cas (FIG. 9D, left panel: positions 2, 3 and 4, 5; right panel: positions 3, 4 and 5, 7).

FIG. 10 shows activity of Type II Cas proteins against an EGFP reporter in mammalian cells (Example 2). The activity of the selected Type II Cas proteins was evaluated after transient electroporation of plasmids encoding each nuclease together with the indicated guide RNAs in U2OS cells stably expressing EGFP. For each Type II Cas protein, three different sgRNAs targeting the EGFP coding sequence were evaluated. For AIWM and AIWR Type II Cas proteins different spacer lengths were also evaluated, as indicated in the graph legend. Loss of EGFP fluorescence, expressed as % of EGFP-negative cells, was measured by cytofluorimetry. Data presented as mean±SEM of n=2 biologically independent runs.

FIG. 11 shows a schematic representation of the rs7984 locus with the position of the EQSC, AHZW and BDLP Type II Cas guide RNAs used in the study of Example 3. Figure discloses SEQ ID NOS 341, 343, 340, 455, 339, 338, 337, 344, and 342, respectively, in order of appearance.

FIG. 12 shows the editing activity of EQSC, AHZW and BDLP Type II Cas proteins towards the rs7984A RHO SNP allele after transient plasmid transfection in HEK293T cells (homozygous rs7984A). Data reported as mean±SEM for n=2 independent runs.

FIGS. 13A-13C show the editing activity of BDLP and EQSC Type II Cas in combination with panels of sgRNAs targeting the TRAC (FIG. 13A), B2M (FIG. 13B), PD-1 (FIG. 13C) after transient plasmid transfection in HEK293T cells (Example 4). Data presented as mean±SEM for n≥2 independent runs.

### 6. DETAILED DESCRIPTION

In one aspect, the disclosure provides Type II Cas proteins (e.g., AHZW Type II Cas proteins, ABSE Type II Cas proteins, AIXM Type II Cas proteins, AXTQ Type II Cas proteins, AIWM Type II Cas proteins, AIWR Type II Cas proteins, AIYQ Type II Cas proteins, EQSC Type II Cas proteins, BDLP Type II Cas proteins, and BDKL Type II Cas proteins). Type II Cas proteins of the disclosure can be in the form of fusion proteins. Unless required otherwise by context, disclosures relating to Type II Cas proteins encompass Type II Cas proteins which are not fusion proteins and Type II Cas proteins which are in the form of fusion proteins (e.g., Type II Cas protein comprising one or more nuclear localization signals and/or one or more tags).

In some embodiments, a Type II Cas protein of the disclosure comprises an amino acid sequence having at least 50% (e.g., at least 55%, at least 60%, at least 65%, at least 70%, at least 75%, at least 85%, at least 90%, at least 95%, or more) sequence identity to a RuvC-I domain, RuvC-II domain, RuvC-III domain, BH domain, REC domain, HNH domain, WED domain, or PID domain of an AHZW Type II Cas protein, ABSE Type II Cas protein, AIXM Type II Cas protein, AXTQ Type II Cas protein, AIWM Type II Cas protein, AIWR Type II Cas protein, AIYQ Type II Cas protein, EQSC Type II Cas protein, BDLP Type II Cas protein, or a BDKL Type II Cas protein. In some embodiments, a Type II Cas protein of the disclosure is a chimeric Type II Cas protein, for example, comprising one or more domains from a AHZW Type II Cas protein and/or ABSE Type II Cas protein and/or AIXM Type II Cas protein and/or AXTQ Type II Cas protein and/or AIWM Type II Cas protein and/or AIWR Type II Cas protein and/or AIYQ Type II Cas protein and/or EQSC Type II Cas protein and/or BDLP Type II Cas protein and/or a BDKL Type II Cas protein, and one or more domains from a different Type II Cas protein such as SpCas9.

Exemplary features of Type II Cas proteins of the disclosure are described in Section 6.2.

In another aspect, the disclosure provides guide (gRNA) molecules, for example single guide RNAs (sgRNAs), and combinations of guide RNA molecules. Exemplary features of the gRNAs and combinations of gRNAs of the disclosure are further described in Section 6.3.

In further aspects, the disclosure provides systems comprising a Type II Cas protein of the disclosure and one or more gRNAs, e.g., sgRNAs. Exemplary features of systems are described in Section 6.4.

In further aspects, the disclosure provides nucleic acids and pluralities of nucleic acids encoding a Type II Cas protein of the disclosure and, optionally, a guide RNA, for example a sgRNA, and provides nucleic acids encoding a gRNA, for example a sgRNA, of the disclosure and, optionally, a Type II Cas protein. Exemplary features of nucleic and pluralities of nucleic acids of the disclosure are described in Section 6.5.

In further aspects, the disclosure provides particles comprising the Type II Cas proteins, gRNAs, nucleic acids, and systems of the disclosure. Exemplary features of particles of the disclosure are described in Section 6.6.

In another aspect, the disclosure provides cells and populations of cells containing or contacted with a Type II Cas protein, gRNA, nucleic acid, plurality of nucleic acids, system, or particle of the disclosure. Exemplary features of such cells and cell populations are described in Section 6.6.

In another aspect, the disclosure provides pharmaceutical compositions comprising a Type II Cas protein, gRNA, nucleic acid, plurality of nucleic acids, system, particle, cell, or population of cells together with one or more excipients. Exemplary features of pharmaceutical compositions are described in Section 6.7.

In another aspect, the disclosure provides methods of altering cells (e.g., editing the genome of a cell) using the Type II Cas proteins, gRNAs, nucleic acids, systems, particles, and pharmaceutical compositions of the disclosure. Features of exemplary methods of altering cells are described in Section 6.8.

Those skilled in the relevant art will recognize and appreciate that many changes can be made to the various embodiments described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### 6.1. Definitions

Unless defined otherwise, all technical and scientific terms used herein have the same meaning as commonly understood to one of ordinary skill in the art to which this invention belongs. The following definitions are provided for the full understanding of terms used in this specification.

As used in the specification and claims, the singular form “a,” “an,” and “the” include plural references unless the context clearly dictates otherwise. For example, the term “an agent” includes a plurality of agents, including mixtures thereof.

Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

A Type II Cas protein refers to a wild-type or engineered Type II Cas protein. Engineered Type II Cas proteins can also be referred to as Type II Cas variants. For the avoidance of doubt, any disclosure pertaining to a “Type II Cas” or “Type II Cas protein” pertains to wild-type Type II Cas proteins and Type II Cas variants, unless the context dictates otherwise. A Type II Cas protein can have nuclease activity or be catalytically inactive (e.g., as in a dCas).

As used herein, the percentage identity between two nucleotide sequences or between two amino acid sequences is calculated by multiplying the number of matches between a pair of aligned sequences by 100, and dividing by the length of the aligned region. Identity scoring only counts perfect matches and does not consider the degree of similarity of amino acids to one another, nor does it consider substitutions or deletions as matches. Alignment for purposes of determining percent sequence identity can be achieved in various ways that are within the skill in the art, for instance, by manual alignment or using publicly available computer software such as BLAST, BLAST-2, ALIGN, ALIGN-2 or Megalign (DNASTAR) software. Those skilled in the art can determine appropriate parameters for achieving maximum alignment.

Guide RNA molecule (gRNA) refers to an RNA capable of forming a complex with a Type II Cas protein and which can direct the Type II Cas protein to a target DNA. gRNAs typically comprise a spacer of 15 to 30 nucleotides in length. gRNAs of the disclosure are in some embodiments single guide RNAs (sgRNAs), which typically comprise a spacer at the 5′ end of the molecule and a 3′ sgRNA scaffold. Various non-limiting examples of 3′ sgRNA scaffolds are described in Section 6.3.

An sgRNA can in some embodiments comprise no uracil base at the 3′ end of the sgRNA sequence. Alternatively, a sgRNA can comprise one or more uracil bases at the 3′ end of the sgRNA sequence. For example, a sgRNA can comprise 1 uracil (U) at the 3′ end of the sgRNA sequence, 2 uracil (UU) at the 3′ end of the sgRNA sequence, 3 uracil (UUU) at the 3′ end of the sgRNA sequence, 4 uracil (UUUU) at the 3′ end of the sgRNA sequence, 5 uracil (UUUUU) at the 3′ end of the sgRNA sequence, 6 uracil (UUUUUU) at the 3′ end of the sgRNA sequence, 7 uracil (UUUUUUU) at the 3′ end of the sgRNA sequence, or 8 uracil (UUUUUUUU) at the 3′ end of the sgRNA sequence. Different length stretches of uracil can be appended at the 3′ end of a sgRNA as terminators. Thus, for example, the 3′ sgRNA scaffolds set forth in Section 6.3 can be modified by adding or removing one or more uracils at the end of the sequence.

Peptide, protein, and polypeptide are used interchangeably to refer to a natural or synthetic molecule comprising two or more amino acids linked by the carboxyl group of one amino acid to the alpha amino group of another. The amino acids may be natural or synthetic, and can contain chemical modifications such as disulfide bridges, substitution of radioisotopes, phosphorylation, substrate chelation (e.g., chelation of iron or copper atoms), glycosylation, acetylation, formylation, amidation, biotinylation, and a wide range of other modifications. A polypeptide may be attached to other molecules, for instance molecules required for function. Examples of molecules which may be attached to a polypeptide include, without limitation, cofactors, polynucleotides, lipids, metal ions, phosphate, etc. Non-limiting examples of polypeptides include peptide fragments, denatured/unstructured polypeptides, polypeptides having quaternary or aggregated structures, etc. There is expressly no requirement that a polypeptide must contain an intended function; a polypeptide can be functional, non-functional, function for unexpected/unintended purposes, or have unknown function. A polypeptide is comprised of approximately twenty, standard naturally occurring amino acids, although natural and synthetic amino acids which are not members of the standard twenty amino acids may also be used. The standard twenty amino acids include alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

Polynucleotide and oligonucleotide are used interchangeably and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, primers and gRNAs. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A polynucleotide is composed of a specific sequence of four nucleotide bases: adenine (A); cytosine (C); guanine (G); thymine (T); and uracil (U) for thymine (T) when the polynucleotide is RNA. Thus, the term “nucleotide sequence” is the alphabetical representation of a polynucleotide molecule. The letters used in polynucleotide sequences described herein correspond to IUPAC notation. For example, the letter “N” in a nucleotide sequence represents a nucleotide which can be A, T, C, or G in a DNA sequence, or A, U, C, or G in a RNA sequence; the letter “R” in a nucleotide sequence represents a nucleotide which can be A or G; and the letter “V” in a nucleotide sequence represents a nucleotide which can be “A, C, or G.

Protospacer adjacent motif (PAM) refers to a DNA sequence downstream (e.g., immediately downstream) of a target sequence on the non-target strand recognized by a Type II Cas protein. A PAM sequence is located 3′ of the target sequence on the non-target strand.

Spacer refers to a region of a gRNA molecule which is partially or fully complementary to a target sequence found in the + or − strand of genomic DNA. When complexed with a Type II Cas protein, the gRNA directs the Type II Cas to the target sequence in the genomic DNA. A spacer of a Type II Cas gRNA is typically 15 to 30 nucleotides in length (e.g., 20-25 nucleotides). The nucleotide sequence of a spacer can be, but is not necessarily, fully complementary to the target sequence. For example, a spacer can contain one or more mismatches with a target sequence, e.g., the spacer can comprise one, two, or three mismatches with the target sequence.

### 6.2. Type II Cas Proteins

### 6.2.1. Type IIA Cas Proteins

### 6.2.1.1. AHZW Type II Cas Proteins

In one aspect, the disclosure provides AHZW Type II Cas proteins. AHZW Type II Cas proteins can be further classified as Type IIA Cas proteins. The AHZW Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:1. In some embodiments, the AHZW Type II Cas proteins comprise an amino acid sequence that is at least 95%, at least 96%, at least 97%, at least 98%, or at least 99% identical to SEQ ID NO:1. In some embodiments, a AHZW Type II Cas protein comprises an amino acid sequence that is identical to SEQ ID NO:1.

Exemplary AHZW Type II Cas protein sequences and nucleotide sequences encoding exemplary AHZW Type II Cas proteins are set forth in Table 1A.

TABLE 1A
AHZW Type II Cas Sequences

| | | SEQ ID |
|---|---|---|
| Name | Sequence | NO |
| AHZW Type II | EKRNNGLVLGLDIGVTSVGYGIVNRETGEAVDFGVRLFSEWDPANNQNR | 1 |
| Cas amino | RAKRSGRRLRSRRKNRLQDMKKFLVDQGMIPSDYAFHLDPYALRVKGL | |
| acid | SEKLTDEELATVLYNLAKLRGSSLETVEDTDDTKSDDSLSTKNNLRINDA | |
| sequence | LIKSGKYVCQIQQERLSEKEKLRGHINNFRTKDYVSELKQILSNQGKSKD | |
| (without N- | FNDYVENLIVRRRDFSDGPGSQKSPTPYGRWLTKDQEEPIDLIEKMRGK | |
| terminal | CSYYPDQLRCAKMAYKADLFNLLNDLNNLSVKGEKLERDQKEQVIEYVN | |
| methionine) | EKGSIKPKQLAKLLGLSDIDDISGFRIDKSDKPILTEFKGYIKVKKVLENSG | |
| | LYHDHELVDQIIDVLTRDKVVERRANEIRKLSNKFSEEEIEALANLTQISGY | |
| | HSMSAKAINEFNKEMLISPLNQMQIITNRGNQNDRFNKLKGMKEIHADDT | |
| | AILSPVAKRAQREAMKVVNRLRELYGEFDSIVVETTRAKNSQEQKTKIKE | |
| | SQAFFEKEKKRAQQIVDENKYNLTVNAKLIQKLRLYTEQNYKTAYALVPID | |
| | LDLLISDPTAFEVDHIIPISISYDDSFSNKVLVTRWENQIKGNLTPLQAHKN | |
| | HKFGEYSDLASYIANVNDLRKMNGMRYEKKKNLLLSDEDLTKFDTQKKFI | |
| | ARNIVDTAYAERVVLNTLQDYFRANEISTVVNTVKGNLTHMFRNKIHLDK | |
| | ERSDIDTGDAHHAIDALIIASLKVFRRFGEILDKRPETIIDEETGEVLEIIEDK | |
| | KFFDPRYIEIVHKLSLIEKDVTKFSYKIDTKPNRQVADETIYSTRNINGEDL | |
| | VVKKYKNIYEEKFVTLANDILENNVEKYLMYRNSPETFKYISDIILYYAKEY | |
| | SDDEKLMKNGLFVKNPLNKYYEEHHEYLHKPSKHNNGPSIISMKYTDGR | |
| | LGNKIDISKNYSVDTEKKKVVLQQISPYRTDFYYSPIEGYRFVTIRYSNVY | |
| | YSSTQGLYIIEKDWYKSQKKSKKIDDSFEFCFSMHRNEIIRIKKEDEDMM | |
| | WRFTATNNDSTNKIEVKPIGHKINKQLILAIGKKIILLEKYATDVCGNLYKVT | |
| | NQELKLEFK | |
| AHZW Type II | MEKRNNGLVLGLDIGVTSVGYGIVNRETGEAVDFGVRLFSEWDPANNQ | 2 |
| Cas amino | NRRAKRSGRRLRSRRKNRLQDMKKFLVDQGMIPSDYAFHLDPYALRVK | |
| acid | GLSEKLTDEELATVLYNLAKLRGSSLETVEDTDDTKSDDSLSTKNNLRIN | |
| sequence | DALIKSGKYVCQIQQERLSEKEKLRGHINNFRTKDYVSELKQILSNQGKS | |
| | KDFNDYVENLIVRRRDFSDGPGSQKSPTPYGRWLTKDQEEPIDLIEKMR | |
| | GKCSYYPDQLRCAKMAYKADLFNLLNDLNNLSVKGEKLERDQKEQVIEY | |
| | VNEKGSIKPKQLAKLLGLSDIDDISGFRIDKSDKPILTEFKGYIKVKKVLEN | |
| | SGLYHDHELVDQIIDVLTRDKVVERRANEIRKLSNKFSEEEIEALANLTQIS | |
| | GYHSMSAKAINEFNKEMLISPLNQMQIITNRGNQNDRFNKLKGMKEIHAD | |
| | DTAILSPVAKRAQREAMKVVNRLRELYGEFDSIVVETTRAKNSQEQKTKI | |
| | KESQAFFEKEKKRAQQIVDENKYNLTVNAKLIQKLRLYTEQNYKTAYALV | |
| | PIDLDLLISDPTAFEVDHIIPISISYDDSFSNKVLVTRWENQIKGNLTPLQAH | |
| | KNHKFGEYSDLASYIANVNDLRKMNGMRYEKKKNLLLSDEDLTKFDTQK | |
| | KFIARNIVDTAYAERVVLNTLQDYFRANEISTVVNTVKGNLTHMFRNKIHL | |
| | DKERSDIDTGDAHHAIDALIIASLKVFRRFGEILDKRPETIIDEETGEVLEIIE | |
| | DKKFFDPRYIEIVHKLSLIEKDVTKFSYKIDTKPNRQVADETIYSTRNINGE | |
| | DLVVKKYKNIYEEKFVTLANDILENNVEKYLMYRNSPETFKYISDIILYYAK | |
| | EYSDDEKLMKNGLFVKNPLNKYYEEHHEYLHKPSKHNNGPSIISMKYTD | |
| | GRLGNKIDISKNYSVDTEKKKVVLQQISPYRTDFYYSPIEGYRFVTIRYSN | |
| | VYYSSTQGLYIIEKDWYKSQKKSKKIDDSFEFCFSMHRNEIIRIKKEDEDM | |
| | MWRFTATNNDSTNKIEVKPIGHKINKQLILAIGKKIILLEKYATDVCGNLYK | |
| | VTNQELKLEFK | |
| AHZW Type II | MIPNPLLGLDKRTADGSEFESPKKKRKVGSEKRNNGLVLGLDIGVTSVG | 3 |
| Cas | YGIVNRETGEAVDFGVRLFSEWDPANNQNRRAKRSGRRLRSRRKNRLQ | |
| mammalian | DMKKFLVDQGMIPSDYAFHLDPYALRVKGLSEKLTDEELATVLYNLAKLR | |
| expression | GSSLETVEDTDDTKSDDSLSTKNNLRINDALIKSGKYVCQIQQERLSEKE | |
| construct | KLRGHINNFRTKDYVSELKQILSNQGKSKDFNDYVENLIVRRRDFSDGPG | |
| (includes N- | SQKSPTPYGRWLTKDQEEPIDLIEKMRGKCSYYPDQLRCAKMAYKADLF | |
| terminal SV5 | NLLNDLNNLSVKGEKLERDQKEQVIEYVNEKGSIKPKQLAKLLGLSDIDDI | |
| tag and NLS | SGFRIDKSDKPILTEFKGYIKVKKVLENSGLYHDHELVDQIIDVLTRDKVVE | |
| and C- | RRANEIRKLSNKFSEEEIEALANLTQISGYHSMSAKAINEFNKEMLISPLN | |
| terminal NLS) | QMQIITNRGNQNDRFNKLKGMKEIHADDTAILSPVAKRAQREAMKVVNR | |
| (aa) | LRELYGEFDSIVVETTRAKNSQEQKTKIKESQAFFEKEKKRAQQIVDENK | |
| | YNLTVNAKLIQKLRLYTEQNYKTAYALVPIDLDLLISDPTAFEVDHIIPISISY | |
| | DDSFSNKVLVTRWENQIKGNLTPLQAHKNHKFGEYSDLASYIANVNDLR | |
| | KMNGMRYEKKKNLLLSDEDLTKFDTQKKFIARNIVDTAYAERVVLNTLQD | |
| | YFRANEISTVVNTVKGNLTHMFRNKIHLDKERSDIDTGDAHHAIDALIIASL | |
| | KVFRRFGEILDKRPETIIDEETGEVLEIIEDKKFFDPRYIEIVHKLSLIEKDVT | |
| | KFSYKIDTKPNRQVADETIYSTRNINGEDLVVKKYKNIYEEKFVTLANDILE | |
| | NNVEKYLMYRNSPETFKYISDIILYYAKEYSDDEKLMKNGLFVKNPLNKY | |
| | YEEHHEYLHKPSKHNNGPSIISMKYTDGRLGNKIDISKNYSVDTEKKKVV | |
| | LQQISPYRTDFYYSPIEGYRFVTIRYSNVYYSSTQGLYIIEKDWYKSQKKS | |
| | KKIDDSFEFCFSMHRNEIIRIKKEDEDMMWRFTATNNDSTNKIEVKPIGHK | |
| | INKQLILAIGKKIILLEKYATDVCGNLYKVTNQELKLEFKSRKRTADGSEFE | |
| | SPKKKRKV | |
| AHZW Type II | ATGGAGAAAAGAAATAATGGTTTAGTTTTAGGGTTAGACATCGGTGTT | 4 |
| Cas coding | ACTTCAGTTGGTTATGGAATTGTAAATCGTGAAACTGGAGAAGCAGTT | |
| sequence (nt) | GATTTTGGTGTGCGCTTATTTAGCGAATGGGATCCAGCAAATAACCAA | |
| (not codon | AATAGACGTGCAAAGAGAAGTGGCAGAAGACTTCGCTCTAGAAGGAA | |
| optimized) | AAATCGATTACAAGATATGAAAAAATTTCTTGTTGATCAAGGATGATTC | |
| | CTTCTGATTATGCTTTTCATCTAGATCCATATGCATTAAGAGTTAAAGG | |
| | CTTATCAGAGAAATTGACAGATGAAGAACTTGCAACAGTTCTATACAA | |
| | CTTAGCAAAATTACGTGGAAGTAGTTTGGAGACTGTAGAAGATACAGA | |
| | TGATACTAAGAGTGATGATTCTCTAAGTACGAAAAATAATTTAAGAATT | |
| | AATGATGCATTAATCAAGAGTGGAAAATATGTTTGTCAAATTCAGCAA | |
| | GAAAGATTATCTGAAAAGGAAAAGTTACGCGGACATATTAACAACTTC | |
| | CGTACAAAGGATTATGTTTCAGAATTAAAACAAATCTTATCAAATCAGG | |
| | GTAAGTCAAAAGATTTTAATGATTATGTAGAAAACTTAATAGTGCGTAG | |
| | AAGAGATTTTTCTGACGGACCTGGTTCCCAGAAATCTCCTACACCTTA | |
| | TGGACGTTGGTTAACAAAAGATCAAGAAGAACCAATAGACTTGATTGA | |
| | GAAGATGAGAGGTAAGTGCTCATACTATCCTGATCAATTACGTTGTGC | |
| | AAAAATGGCTTATAAAGCTGATTTATTTAATTTGCTAAACGACTTGAAT | |
| | AACTTAAGTGTCAAAGGTGAAAAACTTGAAAGAGATCAAAAAGAACAA | |
| | GTCATTGAATACGTTAATGAAAAGGGTTCTATCAAGCCAAAACAATTA | |
| | GCCAAACTATTAGGACTCAGTGATATTGATGATATTTCTGGTTTTAGAA | |
| | TAGATAAGAGTGATAAGCCAATACTCACTGAATTCAAAGGCTATATAA | |
| | AAGTTAAGAAGGTTCTTGAGAATTCGGGTTTGTATCATGACCATGAAC | |
| | TTGTTGATCAGATAATTGATGTTTTAACACGTGATAAGGTAGTTGAAA | |
| | GACGTGCGAATGAAATTCGTAAACTATCAAATAAGTTTAGTGAAGAGG | |
| | AGATAGAGGCATTAGCTAACCTAACACAGATTTCTGGATACCATTCTA | |
| | TGTCAGCAAAGGCAATTAATGAGTTTAATAAAGAGATGCTTATTTCAC | |
| | CATTAAACCAGATGCAAATTATTACGAATAGAGGTAATCAGAACGACC | |
| | GCTTTAATAAACTAAAAGGAATGAAAGAGATCCATGCAGATGATACTG | |
| | CAATTTTAAGTCCAGTTGCTAAGAGAGCACAAAGAGAAGCAATGAAAG | |
| | TCGTTAATAGACTTAGAGAACTCTATGGTGAATTTGATAGTATTGTTGT | |
| | AGAGACAACACGTGCAAAAAATAGCCAGGAACAAAAGACAAAAATTAA | |
| | AGAGTCACAAGCTTTCTTTGAAAAAGAAAAGAAACGTGCCCAACAAAT | |
| | AGTCGATGAAAATAAATATAATCTAACAGTTAATGCAAAGCTAATTCAG | |
| | AAGTTACGTTTATATACAGAACAGAATTATAAGACTGCGTATGCACTA | |
| | GTGCCAATTGATTTAGATTTATTAATTAGTGATCCGACAGCTTTTGAG | |
| | GTAGATCATATTATTCCAATCTCTATTTCCTATGATGATTCGTTCTCAA | |
| | ATAAGGTTCTTGTAACTCGTTGGGAGAATCAGATTAAAGGTAATCTAA | |
| | CACCACTACAGGCACATAAGAATCATAAGTTTGGAGAGTATTCAGATT | |
| | TAGCAAGTTATATTGCAAACGTCAATGACTTACGCAAGATGAATGGAA | |
| | TGCGATATGAGAAAAAGAAAAATCTCTTGTTATCAGATGAGGATTTGA | |
| | CGAAGTTTGATACACAAAAGAAATTTATTGCTAGAAATATTGTGGATAC | |
| | TGCATATGCAGAAAGAGTTGTTTTAAATACATTGCAAGACTACTTCCG | |
| | TGCTAATGAAATTTCTACTGTTGTAAATACCGTAAAAGGTAATTTAACG | |
| | CACATGTTTAGAAATAAGATTCATCTTGATAAGGAGAGAAGTGATATT | |
| | GATACTGGTGATGCACATCACGCTATTGATGCATTGATTATTGCTTCC | |
| | TTAAAGGTCTTCCGCCGTTTCGGAGAAATCCTTGATAAGCGACCAGA | |
| | AACAATTATTGATGAAGAAACAGGGGAAGTTTTAGAAATTATTGAGGA | |
| | TAAGAAATTCTTTGATCCACGGTATATAGAAATTGTTCATAAACTTTCA | |
| | CTGATAGAAAAGGATGTAACGAAGTTTTCTTACAAGATTGATACTAAA | |
| | CCTAATCGACAAGTAGCTGACGAAACAATCTATTCTACAAGAAATATT | |
| | AATGGGGAAGATCTTGTTGTTAAGAAATATAAAAACATTTACGAAGAA | |
| | AAGTTTGTTACATTAGCAAATGATATTTTAGAAAATAATGTTGAAAAAT | |
| | ATCTGATGTATCGCAATAGTCCAGAAACATTTAAGTATATATCTGATAT | |
| | TATTCTGTATTATGCAAAAGAATATAGTGACGATGAGAAACTTATGAA | |
| | GAATGGATTATTTGTAAAGAACCCATTAAATAAATATTACGAAGAACAT | |
| | CATGAGTATCTACATAAGCCATCGAAACATAATAATGGACCATCGATT | |
| | ATTTCTATGAAATATACAGATGGAAGATTAGGAAATAAGATAGATATTT | |
| | CGAAGAACTATTCAGTAGATACAGAAAAGAAAAAGGTAGTCCTTCAAC | |
| | AGATTTCTCCATATCGTACAGACTTCTATTATTCTCCTATAGAAGGATA | |
| | TAGATTCGTGACAATAAGATACTCAAATGTTTATTATTCCTCCACCCAA | |
| | GGTTTATATATCATTGAAAAAGATTGGTATAAATCCCAAAAGAAATCAA | |
| | AGAAGATTGATGATTCATTTGAATTCTGTTTCTCCATGCATAGAAATGA | |
| | AATTATTCGCATCAAGAAAGAAGATGAAGATATGATGTGGAGATTTAC | |
| | AGCAACAAATAATGACTCAACGAATAAAATTGAAGTTAAACCAATAGG | |
| | GCATAAAATTAATAAGCAGTTAATTCTAGCAATTGGAAAGAAGATTATT | |
| | TTATTAGAAAAATATGCAACTGACGTATGTGGAAATCTATATAAAGTAA | |
| | CAAATCAGGAGTTGAAACTAGAATTTAAATGA | |
| AHZW Type II | GAAAAGCGGAACAACGGCTTAGTGCTGGGACTGGATATCGGCGTGA | 5 |
| Cas coding | CCAGCGTGGGCTACGGCATCGTGAACAGAGAAACCGGCGAGGCCGT | |
| sequence (nt) | GGACTTTGGTGTTAGACTGTTCAGCGAATGGGACCCCGCCAACAACC | |
| (human | AAAACAGAAGAGCGAAGCGAAGCGGCAGACGGCTGAGAAGCAGACG | |
| codon- | GAAGAACAGACTGCAGGACATGAAGAAGTTCCTGGTGGACCAGGGA | |
| optimized) | ATGATCCCTAGCGATTACGCTTTCCACCTGGACCCTTACGCCCTGAG | |
| | AGTGAAGGGACTGTCCGAGAAGCTGACCGACGAGGAACTGGCCACG | |
| | GTGCTGTATAATCTGGCAAAGCTCCGGGGCAGCAGCCTGGAAACCG | |
| | TGGAAGATACCGATGATACCAAGTCTGATGACAGCCTATCCACCAAG | |
| | AATAACCTGAGGATTAACGACGCCCTGATAAAGTCTGGCAAGTACGT | |
| | GTGTCAGATCCAGCAGGAGCGGCTGTCTGAAAAGGAAAAACTGCGG | |
| | GGCCACATCAATAACTTCCGGACAAAGGACTACGTGAGCGAACTGAA | |
| | GCAGATCCTGAGCAATCAGGGCAAAAGCAAGGACTTTAACGATTACG | |
| | TTGAGAACCTGATCGTGCGGAGAAGAGATTTCAGCGATGGCCCCGG | |
| | CTCTCAGAAAAGTCCTACACCTTACGGCCGGTGGCTGACAAAGGACC | |
| | AAGAGGAACCTATCGACCTGATCGAGAAGATGCGGGGAAAGTGCAG | |
| | CTACTATCCTGACCAGCTGCGGTGCGCCAAGATGGCCTACAAGGCC | |
| | GATCTGTTCAACCTGCTGAACGACCTGAACAACCTGAGTGTGAAAGG | |
| | CGAAAAGCTGGAACGCGATCAGAAGGAACAGGTGATAGAATACGTGA | |
| | ACGAGAAAGGTTCCATCAAACCTAAGCAGCTGGCCAAACTGCTGGGC | |
| | CTGAGCGACATCGATGACATCTCTGGCTTCCGGATCGATAAGTCCGA | |
| | CAAGCCGATACTGACCGAGTTTAAGGGATACATCAAGGTGAAGAAGG | |
| | TGCTGGAAAACAGCGGCCTGTACCACGACCACGAGCTGGTGGACCA | |
| | GATCATCGATGTGCTGACCAGAGACAAAGTGGTCGAGCGTAGGGCC | |
| | AACGAGATCCGGAAGCTGAGTAACAAATTCTCCGAGGAGGAAATCGA | |
| | AGCCCTGGCCAACCTGACCCAGATCAGCGGCTACCACAGCATGAGC | |
| | GCCAAAGCCATCAACGAGTTCAACAAGGAGATGCTGATCTCCCCACT | |
| | GAACCAGATGCAGATCATAACAAACAGAGGAAACCAGAACGACAGAT | |
| | TCAATAAGCTGAAAGGAATGAAGGAGATCCACGCCGACGACACAGCC | |
| | ATCCTGAGCCCCGTGGCCAAGAGAGCCCAGAGAGAGGCCATGAAAG | |
| | TGGTGAACAGACTACGCGAGCTTTATGGCGAGTTCGACAGCATCGTG | |
| | GTCGAGACAACCAGAGCTAAGAACAGCCAGGAGCAGAAAACCAAAAT | |
| | CAAGGAGAGCCAGGCCTTCTTCGAGAAAGAGAAAAAGAGAGCTCAG | |
| | CAGATCGTGGATGAGAACAAGTACAACCTGACCGTCAACGCCAAACT | |
| | CATCCAGAAACTGCGGCTTTACACCGAGCAGAATTACAAGACCGCCT | |
| | ACGCCCTGGTGCCCATCGACCTGGACCTGCTGATCAGCGACCCTAC | |
| | CGCCTTCGAGGTGGACCACATCATCCCCATCAGCATCAGCTACGACG | |
| | ACAGCTTTTCTAATAAGGTGCTGGTCACCCGGTGGGAGAATCAGATC | |
| | AAGGGAAACCTGACCCCTCTGCAAGCCCACAAGAACCACAAGTTCGG | |
| | CGAGTACAGCGACTTAGCCAGCTACATCGCCAACGTGAACGACCTGC | |
| | GGAAAATGAACGGAATGAGATACGAGAAGAAGAAGAACCTGCTGCTG | |
| | TCTGACGAGGATCTGACCAAATTCGATACCCAGAAGAAGTTCATCGC | |
| | TAGAAACATCGTGGACACCGCTTATGCCGAGAGAGTGGTGCTGAATA | |
| | CCCTGCAAGATTACTTCAGAGCCAACGAAATCAGCACAGTGGTGAAT | |
| | ACAGTGAAGGGCAACCTGACCCACATGTTCCGGAACAAGATCCACCT | |
| | GGACAAGGAGAGATCTGACATTGATACAGGCGACGCCCATCATGCCA | |
| | TCGACGCTCTGATCATCGCTTCTCTCAAAGTCTTTCGGAGATTTGGCG | |
| | AAATCCTGGACAAGCGCCCCGAGACAATCATTGATGAAGAAACCGGC | |
| | GAAGTGCTGGAAATCATCGAGGACAAGAAGTTCTTTGACCCTAGATA | |
| | CATCGAGATCGTGCACAAGCTGTCTCTGATCGAGAAAGATGTGACCA | |
| | AGTTCAGCTACAAGATCGACACCAAGCCCAACAGACAGGTGGCCGAC | |
| | GAGACAATCTACTCTACAAGAAACATCAACGGCGAGGACCTCGTGGT | |
| | CAAGAAGTACAAGAACATCTACGAGGAAAAATTCGTGACACTGGCCA | |
| | ACGACATCCTGGAAAACAATGTGGAAAAGTACCTGATGTACAGAAATA | |
| | GCCCTGAAACCTTCAAGTACATCAGCGACATCATCCTGTATTACGCCA | |
| | AGGAGTACTCTGATGATGAAAAGCTGATGAAAAACGGCTTGTTCGTG | |
| | AAAAATCCCCTGAACAAGTACTACGAGGAACACCACGAGTACCTGCA | |
| | CAAGCCTAGCAAGCACAACAATGGCCCTAGTATCATCAGCATGAAGT | |
| | ATACAGATGGCCGGCTGGGCAACAAGATCGACATCAGCAAGAACTAC | |
| | TCTGTGGACACCGAGAAGAAGAAGGTGGTGCTGCAACAGATCAGCC | |
| | CTTACCGGACCGACTTCTACTACAGCCCTATCGAGGGCTACCGGTTC | |
| | GTGACCATCAGATACAGCAACGTGTACTACAGCAGCACGCAGGGCCT | |
| | GTACATTATCGAGAAGGATTGGTACAAGAGCCAAAAGAAATCCAAAAA | |
| | AATCGACGACTCCTTCGAGTTCTGCTTCAGCATGCACAGGAATGAGA | |
| | TCATCAGAATCAAAAAGGAGGACGAGGATATGATGTGGCGGTTCACT | |
| | GCCACAAACAACGACAGCACAAACAAGATCGAGGTGAAGCCTATCGG | |
| | CCATAAGATCAACAAGCAGCTGATCCTGGCTATTGGAAAGAAGATCAT | |
| | TCTGCTGGAAAAGTACGCTACAGATGTGTGCGGCAACCTGTACAAAG | |
| | TGACCAACCAGGAGCTGAAACTGGAGTTCAAG | |
| AHZW Type II | ATGATCCCCAATCCCCTGCTGGGCCTGGACAAGCGGACTGCCGACG | 6 |
| Cas | GCAGTGAGTTCGAGTCTCCCAAGAAAAAGAGGAAAGTGggatccGAAAA | |
| mammalian | GCGGAACAACGGCTTAGTGCTGGGACTGGATATCGGCGTGACCAGC | |
| expression | GTGGGCTACGGCATCGTGAACAGAGAAACCGGCGAGGCCGTGGACT | |
| construct | TTGGTGTTAGACTGTTCAGCGAATGGGACCCCGCCAACAACCAAAAC | |
| (includes N- | AGAAGAGCGAAGCGAAGCGGCAGACGGCTGAGAAGCAGACGGAAG | |
| terminal SV5 | AACAGACTGCAGGACATGAAGAAGTTCCTGGTGGACCAGGGAATGAT | |
| tag and NLS | CCCTAGCGATTACGCTTTCCACCTGGACCCTTACGCCCTGAGAGTGA | |
| and C- | AGGGACTGTCCGAGAAGCTGACCGACGAGGAACTGGCCACGGTGCT | |
| terminal NLS) | GTATAATCTGGCAAAGCTCCGGGGCAGCAGCCTGGAAACCGTGGAA | |
| (nt) | GATACCGATGATACCAAGTCTGATGACAGCCTATCCACCAAGAATAAC | |
| | CTGAGGATTAACGACGCCCTGATAAAGTCTGGCAAGTACGTGTGTCA | |
| | GATCCAGCAGGAGCGGCTGTCTGAAAAGGAAAAACTGCGGGGCCAC | |
| | ATCAATAACTTCCGGACAAAGGACTACGTGAGCGAACTGAAGCAGAT | |
| | CCTGAGCAATCAGGGCAAAAGCAAGGACTTTAACGATTACGTTGAGA | |
| | ACCTGATCGTGCGGAGAAGAGATTTCAGCGATGGCCCCGGCTCTCA | |
| | GAAAAGTCCTACACCTTACGGCCGGTGGCTGACAAAGGACCAAGAG | |
| | GAACCTATCGACCTGATCGAGAAGATGCGGGGAAAGTGCAGCTACTA | |
| | TCCTGACCAGCTGCGGTGCGCCAAGATGGCCTACAAGGCCGATCTG | |
| | TTCAACCTGCTGAACGACCTGAACAACCTGAGTGTGAAAGGCGAAAA | |
| | GCTGGAACGCGATCAGAAGGAACAGGTGATAGAATACGTGAACGAG | |
| | AAAGGTTCCATCAAACCTAAGCAGCTGGCCAAACTGCTGGGCCTGAG | |
| | CGACATCGATGACATCTCTGGCTTCCGGATCGATAAGTCCGACAAGC | |
| | CGATACTGACCGAGTTTAAGGGATACATCAAGGTGAAGAAGGTGCTG | |
| | GAAAACAGCGGCCTGTACCACGACCACGAGCTGGTGGACCAGATCA | |
| | TCGATGTGCTGACCAGAGACAAAGTGGTCGAGCGTAGGGCCAACGA | |
| | GATCCGGAAGCTGAGTAACAAATTCTCCGAGGAGGAAATCGAAGCCC | |
| | TGGCCAACCTGACCCAGATCAGCGGCTACCACAGCATGAGCGCCAA | |
| | AGCCATCAACGAGTTCAACAAGGAGATGCTGATCTCCCCACTGAACC | |
| | AGATGCAGATCATAACAAACAGAGGAAACCAGAACGACAGATTCAAT | |
| | AAGCTGAAAGGAATGAAGGAGATCCACGCCGACGACACAGCCATCCT | |
| | GAGCCCCGTGGCCAAGAGAGCCCAGAGAGAGGCCATGAAAGTGGTG | |
| | AACAGACTACGCGAGCTTTATGGCGAGTTCGACAGCATCGTGGTCGA | |
| | GACAACCAGAGCTAAGAACAGCCAGGAGCAGAAAACCAAAATCAAGG | |
| | AGAGCCAGGCCTTCTTCGAGAAAGAGAAAAAGAGAGCTCAGCAGATC | |
| | GTGGATGAGAACAAGTACAACCTGACCGTCAACGCCAAACTCATCCA | |
| | GAAACTGCGGCTTTACACCGAGCAGAATTACAAGACCGCCTACGCCC | |
| | TGGTGCCCATCGACCTGGACCTGCTGATCAGCGACCCTACCGCCTTC | |
| | GAGGTGGACCACATCATCCCCATCAGCATCAGCTACGACGACAGCTT | |
| | TTCTAATAAGGTGCTGGTCACCCGGTGGGAGAATCAGATCAAGGGAA | |
| | ACCTGACCCCTCTGCAAGCCCACAAGAACCACAAGTTCGGCGAGTAC | |
| | AGCGACTTAGCCAGCTACATCGCCAACGTGAACGACCTGCGGAAAAT | |
| | GAACGGAATGAGATACGAGAAGAAGAAGAACCTGCTGCTGTCTGACG | |
| | AGGATCTGACCAAATTCGATACCCAGAAGAAGTTCATCGCTAGAAACA | |
| | TCGTGGACACCGCTTATGCCGAGAGAGTGGTGCTGAATACCCTGCAA | |
| | GATTACTTCAGAGCCAACGAAATCAGCACAGTGGTGAATACAGTGAA | |
| | GGGCAACCTGACCCACATGTTCCGGAACAAGATCCACCTGGACAAG | |
| | GAGAGATCTGACATTGATACAGGCGACGCCCATCATGCCATCGACGC | |
| | TCTGATCATCGCTTCTCTCAAAGTCTTTCGGAGATTTGGCGAAATCCT | |
| | GGACAAGCGCCCCGAGACAATCATTGATGAAGAAACCGGCGAAGTG | |
| | CTGGAAATCATCGAGGACAAGAAGTTCTTTGACCCTAGATACATCGA | |
| | GATCGTGCACAAGCTGTCTCTGATCGAGAAAGATGTGACCAAGTTCA | |
| | GCTACAAGATCGACACCAAGCCCAACAGACAGGTGGCCGACGAGAC | |
| | AATCTACTCTACAAGAAACATCAACGGCGAGGACCTCGTGGTCAAGA | |
| | AGTACAAGAACATCTACGAGGAAAAATTCGTGACACTGGCCAACGAC | |
| | ATCCTGGAAAACAATGTGGAAAAGTACCTGATGTACAGAAATAGCCCT | |
| | GAAACCTTCAAGTACATCAGCGACATCATCCTGTATTACGCCAAGGA | |
| | GTACTCTGATGATGAAAAGCTGATGAAAAACGGCTTGTTCGTGAAAAA | |
| | TCCCCTGAACAAGTACTACGAGGAACACCACGAGTACCTGCACAAGC | |
| | CTAGCAAGCACAACAATGGCCCTAGTATCATCAGCATGAAGTATACA | |
| | GATGGCCGGCTGGGCAACAAGATCGACATCAGCAAGAACTACTCTGT | |
| | GGACACCGAGAAGAAGAAGGTGGTGCTGCAACAGATCAGCCCTTAC | |
| | CGGACCGACTTCTACTACAGCCCTATCGAGGGCTACCGGTTCGTGAC | |
| | CATCAGATACAGCAACGTGTACTACAGCAGCACGCAGGGCCTGTACA | |
| | TTATCGAGAAGGATTGGTACAAGAGCCAAAAGAAATCCAAAAAAATCG | |
| | ACGACTCCTTCGAGTTCTGCTTCAGCATGCACAGGAATGAGATCATC | |
| | AGAATCAAAAAGGAGGACGAGGATATGATGTGGCGGTTCACTGCCAC | |
| | AAACAACGACAGCACAAACAAGATCGAGGTGAAGCCTATCGGCCATA | |
| | AGATCAACAAGCAGCTGATCCTGGCTATTGGAAAGAAGATCATTCTG | |
| | CTGGAAAAGTACGCTACAGATGTGTGCGGCAACCTGTACAAAGTGAC | |
| | CAACCAGGAGCTGAAACTGGAGTTCAAGtctagaAAGCGGACAGCAGA | |
| | CGGCTCCGAATTTGAAAGCCCTAAGAAAAAGAGAAAGGTGTGA | |

In some embodiments an AHZW Type II Cas protein comprises an amino acid sequence of SEQ ID NO:1, SEQ ID NO:2, or SEQ ID NO:3. In some embodiments, an AHZW Type II Cas protein has nickase activity, for example resulting from one or more amino acid substitutions relative to the sequence of SEQ ID NO:1, SEQ ID NO:2, or SEQ ID NO:3. In some embodiments, the one or more amino acid substitutions providing nickase activity is a D13A substitution, wherein the position of the D13A substitution is defined with respect to the amino acid numbering of SEQ ID NO:2. In some embodiments, the one or more amino acid substitutions providing nickase activity is a N589A substitution, wherein the position of the N589A substitution is defined with respect to the amino acid numbering of SEQ ID NO:2. In some embodiments, an AHZW Type II Cas protein is catalytically inactive, for example due to both a D13A substitution and a N589A substitution, where the positions of the substitutions are defined with respect to the amino acid numbering of SEQ ID NO:2.

### 6.2.1.2. ABSE Type II Cas Proteins

In one aspect, the disclosure provides ABSE Type II Cas proteins. The ABSE Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:7. In some embodiments, the ABSE Type II Cas proteins comprise an amino acid sequence that is at least 95%, at least 96%, at least 97%, at least 98%, or at least 99% identical to SEQ ID NO:7. In some embodiments, an ABSE Type II Cas protein comprises an amino acid sequence that is identical to SEQ ID NO:7.

Exemplary ABSE Type II Cas protein sequences and nucleotide sequences encoding exemplary ABSE Type II Cas proteins are set forth in Table 1B.

TABLE 1B
ABSE Type II Cas Sequences

| | | SEQ ID |
|---|---|---|
| Name | Sequence | NO: |
| ABSE Type II | GYILGLDLGITSVGFGIIEEDTYKIIKYGVRLFDEGTADNNLTRRQNRSSRR | 7 |
| Cas amino | LKSRRKNRINAIKYLLLNNNVIPNVNFDILDNVYELRVKGLTQKLTNLELAN | |
| acid | VLVNLAKRRGSSLDVAVDENDNESVVAASALTKNTAELKKNNLYICQAQL | |
| sequence | AKIKSGVKLRNHDNIYKSDDYEKELRQILSNQDLSNELNDKIVEIIMRRRN | |
| (without N- | FSEGPGSYEFPTKYGSYRVDYVDGIKTIKHVNLIEEMRGKCSIFRNEPRIA | |
| terminal | KCSFEACLFNLLNDLNNLTINGNGKITKEQKLQIINDYVIPKGKITIPQLVKY | |
| methionine) | LGVEEKDITGFRKNKSEKNIITTFDTYSKMINFLPKDILNVTAYVDSIIELLTK | |
| | TAWVEERIEEIKKLNLGLNDDIINQLANMTKVNGYHSLSKKAMDIIIPEMIET | |
| | DKNQMQIIHDNNLDKNDCQEKGAKIKFDSSAILSPVTKRVHMQAIQVVNE | |
| | LRKEYGEFDSIVIETTRAKNSSDEKKDIQEDQKRLEEQKRKSDELLIDLDK | |
| | NPDNYNTLTKLKLRLYKEQEGKTIYAGLPIDLDLLLNDPTAYQIEHIIPYAV | |
| | SFDNSMNNKSLASAKENQDKGKRTPWQYFSSGMVSECEGPINDWESF | |
| | EAFVNSLKISKNKKNNLLNQEDVTKFDNMKAFVARNLSDTSYDVRTVMN | |
| | TMQQYFNDNNINTNVFTVKGRLTNIFRSRVGLKKDRDYFIHHAVDALIIAG | |
| | SKNQSVFNNAYLLSSTKDGILIKKTGEVFDYSQDPFDDPRFLNYISSLKKI | |
| | EGKPEDFSYKVDKKTNRQFADQTIYSTRNYDGVDYVVKKYKDIYGKDGE | |
| | SLKKLFDDKKEQTLLMARNDIQTFELFKKIYENYKNEKNPFAKYKDEFGPI | |
| | KKYCKDGKGPEIYQVKYISDKLGNHLDISDKYNVENGNKKVVLLQISPYR | |
| | TDVYQSKETGLYNFITIRRYHIYQENDMNIIKPELYQKLLSDKKIDETYEFK | |
| | FSLNRNDIINLVTIDNQADHIDEMLYRFIGTNNDKTNKIEIRYIEKMTLKQIM | |
| | VSIGKKIVKFEKYNVSPTGKKAKAEKEVLKLKWK | |
| ABSE Type II | MGYILGLDLGITSVGFGIIEEDTYKIIKYGVRLFDEGTADNNLTRRQNRSS | 8 |
| Cas amino | RRLKSRRKNRINAIKYLLLNNNVIPNVNFDILDNVYELRVKGLTQKLTNLEL | |
| acid | ANVLVNLAKRRGSSLDVAVDENDNESVVAASALTKNTAELKKNNLYICQ | |
| sequence | AQLAKIKSGVKLRNHDNIYKSDDYEKELRQILSNQDLSNELNDKIVEIIMR | |
| | RRNFSEGPGSYEFPTKYGSYRVDYVDGIKTIKHVNLIEEMRGKCSIFRNE | |
| | PRIAKCSFEACLFNLLNDLNNLTINGNGKITKEQKLQIINDYVIPKGKITIPQ | |
| | LVKYLGVEEKDITGFRKNKSEKNIITTFDTYSKMINFLPKDILNVTAYVDSII | |
| | ELLTKTAVVEERIEEIKKLNLGLNDDIINQLANMTKVNGYHSLSKKAMDIIIP | |
| | EMIETDKNQMQIIHDNNLDKNDCQEKGAKIKFDSSAILSPVTKRVHMQAI | |
| | QWVNELRKEYGEFDSIVIETTRAKNSSDEKKDIQEDQKRLEEQKRKSDEL | |
| | LIDLDKNPDNYNTLTKLKLRLYKEQEGKTIYAGLPIDLDLLLNDPTAYQIEHI | |
| | IPYAVSFDNSMNNKSLASAKENQDKGKRTPWQYFSSGMVSECEGPIND | |
| | WESFEAFVNSLKISKNKKNNLLNQEDVTKFDNMKAFVARNLSDTSYDVR | |
| | TVMNTMQQYFNDNNINTNVFTVKGRLTNIFRSRVGLKKDRDYFIHHAVD | |
| | ALIIAGSKNQSVFNNAYLLSSTKDGILIKKTGEVFDYSQDPFDDPRFLNYIS | |
| | SLKKIEGKPEDFSYKVDKKTNRQFADQTIYSTRNYDGVDYVVKKYKDIYG | |
| | KDGESLKKLFDDKKEQTLLMARNDIQTFELFKKIYENYKNEKNPFAKYKD | |
| | EFGPIKKYCKDGKGPEIYQVKYISDKLGNHLDISDKYNVENGNKKVVLLQI | |
| | SPYRTDVYQSKETGLYNFITIRRYHIYQENDMNIIKPELYQKLLSDKKIDET | |
| | YEFKFSLNRNDIINLVTIDNQADHIDEMLYRFIGTNNDKTNKIEIRYIEKMTL | |
| | KQIMVSIGKKIVKFEKYNVSPTGKKAKAEKEVLKLKWK | |
| ABSE Type II | MIPNPLLGLDKRTADGSEFESPKKKRKVGSGYILGLDLGITSVGFGIIEED | 9 |
| Cas | TYKIIKYGVRLFDEGTADNNLTRRQNRSSRRLKSRRKNRINAIKYLLLNNN | |
| mammalian | VIPNVNFDILDNVYELRVKGLTQKLTNLELANVLVNLAKRRGSSLDVAVD | |
| expression | ENDNESVVAASALTKNTAELKKNNLYICQAQLAKIKSGVKLRNHDNIYKS | |
| construct | DDYEKELRQILSNQDLSNELNDKIVEIIMRRRNFSEGPGSYEFPTKYGSY | |
| (includes N- | RVDYVDGIKTIKHVNLIEEMRGKCSIFRNEPRIAKCSFEACLFNLLNDLNN | |
| terminal SV5 | LTINGNGKITKEQKLQIINDYVIPKGKITIPQLVKYLGVEEKDITGFRKNKSE | |
| tag and NLS | KNIITTFDTYSKMINFLPKDILNVTAYVDSIIELLTKTAVVEERIEEIKKLNLGL | |
| and C- | NDDIINQLANMTKVNGYHSLSKKAMDIIIPEMIETDKNQMQIIHDNNLDKN | |
| terminal NLS) | DCQEKGAKIKFDSSAILSPVTKRVHMQAIQVVNELRKEYGEFDSIVIETTR | |
| (aa) | AKNSSDEKKDIQEDQKRLEEQKRKSDELLIDLDKNPDNYNTLTKLKLRLY | |
| | KEQEGKTIYAGLPIDLDLLLNDPTAYQIEHIIPYAVSFDNSMNNKSLASAKE | |
| | NQDKGKRTPWQYFSSGMVSECEGPINDWESFEAFVNSLKISKNKKNNL | |
| | LNQEDVTKFDNMKAFVARNLSDTSYDVRTVMNTMQQYFNDNNINTNVF | |
| | TVKGRLTNIFRSRVGLKKDRDYFIHHAVDALIIAGSKNQSVFNNAYLLSST | |
| | KDGILIKKTGEVFDYSQDPFDDPRFLNYISSLKKIEGKPEDFSYKVDKKTN | |
| | RQFADQTIYSTRNYDGVDYVVKKYKDIYGKDGESLKKLFDDKKEQTLLM | |
| | ARNDIQTFELFKKIYENYKNEKNPFAKYKDEFGPIKKYCKDGKGPEIYQVK | |
| | YISDKLGNHLDISDKYNVENGNKKVVLLQISPYRTDVYQSKETGLYNFITI | |
| | RRYHIYQENDMNIIKPELYQKLLSDKKIDETYEFKFSLNRNDIINLVTIDNQ | |
| | ADHIDEMLYRFIGTNNDKTNKIEIRYIEKMTLKQIMVSIGKKIVKFEKYNVSP | |
| | TGKKAKAEKEVLKLKWKSRKRTADGSEFESPKKKRKV | |
| ABSE Type II | ATGGGATATATTTTAGGATTAGATTTAGGAATTACTAGTGTAGGCTTTG | 10 |
| Cas coding | GTATTATTGAGGAGGATACATATAAAATAATAAAATATGGTGTTAGATT | |
| sequence (nt) | ATTTGATGAAGGTACAGCGGATAATAATTTAACAAGAAGACAAAATCG | |
| not codon | TTCATCAAGAAGACTTAAGAGTAGACGTAAAAATAGAATAAATGCGAT | |
| optimized | TAAATATTTATTATTAAATAATAATGTTATACCGAATGTTAATTTTGATA | |
| | TTTTAGACAACGTATATGAATTAAGAGTAAAAGGCCTAACTCAAAAATT | |
| | GACTAATCTAGAGCTTGCTAATGTATTAGTTAATTTAGCTAAAAGACGT | |
| | GGATCATCATTAGATGTTGCTGTAGATGAAAACGATAATGAAAGTGTT | |
| | GTAGCAGCGTCAGCATTAACTAAAAATACTGCAGAACTAAAGAAAAAT | |
| | AATTTATACATTTGTCAGGCTCAATTAGCTAAAATTAAAAGTGGAGTAA | |
| | AGCTTAGAAACCACGATAATATTTATAAAAGTGATGATTACGAAAAAG | |
| | AACTTAGACAAATTCTTTCTAATCAAGATTTATCTAATGAACTAAATGA | |
| | TAAAATAGTTGAAATTATTATGCGAAGAAGAAATTTCTCGGAAGGACC | |
| | AGGTTCTTATGAATTTCCAACTAAATATGGTTCATATCGTGTTGATTAT | |
| | GTTGACGGCATTAAGACGATTAAACATGTAAACTTAATTGAAGAAATG | |
| | CGTGGCAAGTGTTCTATTTTCAGAAATGAGCCACGTATTGCAAAATGT | |
| | AGTTTTGAGGCTTGCTTATTTAATTTATTAAATGATTTAAATAATCTTAC | |
| | AATTAACGGTAATGGAAAAATAACAAAAGAGCAAAAATTACAAATAATT | |
| | AATGATTATGTTATTCCAAAGGGAAAAATAACAATTCCACAATTAGTTA | |
| | AATATTTAGGAGTTGAGGAAAAGGATATTACAGGATTTAGAAAAAATA | |
| | AGTCTGAGAAAAATATAATTACAACATTTGATACATATTCTAAGATGAT | |
| | TAATTTTTTACCTAAGGATATTCTTAATGTAACAGCATATGTAGATTCA | |
| | ATTATTGAATTACTTACTAAAACTGCTGTAGTTGAAGAGAGAATTGAAG | |
| | AAATTAAGAAATTAAACCTTGGATTAAACGACGATATTATTAATCAACT | |
| | TGCAAATATGACCAAGGTTAATGGATATCATTCACTAAGTAAAAAAGC | |
| | AATGGATATTATTATTCCTGAGATGATTGAAACTGATAAAAATCAAATG | |
| | CAAATTATTCATGATAATAATTTAGATAAGAATGATTGTCAAGAAAAAG | |
| | GGGCTAAAATAAAGTTTGATTCATCAGCAATTTTATCACCAGTTACAAA | |
| | GCGTGTTCATATGCAGGCAATTCAGGTTGTCAACGAACTTAGAAAGG | |
| | AATATGGTGAATTTGATTCAATTGTTATTGAAACAACAAGAGCTAAAAA | |
| | CAGTAGTGATGAAAAGAAAGATATCCAAGAGGATCAAAAAAGATTAGA | |
| | AGAACAAAAAAGAAAATCTGATGAACTTCTTATAGATCTTGATAAGAAT | |
| | CCTGATAATTATAATACTTTAACTAAGCTTAAGTTAAGATTATATAAGG | |
| | AACAAGAAGGAAAAACAATTTATGCTGGTTTACCAATTGACCTTGATTT | |
| | ATTGCTTAATGATCCTACTGCTTATCAAATTGAGCATATTATTCCTTAT | |
| | GCTGTATCTTTTGATAATAGTATGAATAATAAGAGTCTTGCATCAGCAA | |
| | AAGAAAATCAAGATAAAGGAAAACGAACACCTTGGCAGTATTTTTCAT | |
| | CTGGTATGGTTTCAGAATGCGAAGGACCAATTAATGATTGGGAGTCC | |
| | TTTGAAGCATTTGTTAATAGTTTAAAGATATCAAAAAATAAAAAAAATAA | |
| | TCTTTTAAATCAAGAAGATGTAACTAAATTTGATAATATGAAGGCGTTT | |
| | GTTGCAAGAAATTTAAGTGATACTAGTTATGATGTTAGAACTGTTATGA | |
| | ATACGATGCAACAATATTTTAATGATAATAATATTAATACAAATGTATTT | |
| | ACAGTCAAGGGAAGATTAACCAATATTTTCAGAAGCAGAGTAGGGCTT | |
| | AAAAAGGATAGAGATTATTTTATTCATCATGCAGTAGATGCATTAATCA | |
| | TTGCTGGTTCTAAAAATCAGTCAGTTTTCAATAATGCATATTTATTATC | |
| | ATCGACAAAAGATGGAATACTTATTAAAAAAACAGGAGAGGTATTTGA | |
| | TTATTCTCAAGATCCGTTTGATGATCCTAGGTTTTTAAATTATATTTCAT | |
| | CACTTAAAAAAATTGAAGGAAAGCCTGAAGATTTTTCATATAAGGTTG | |
| | ATAAGAAGACAAATAGACAATTTGCAGATCAAACAATTTATTCAACAAG | |
| | AAATTATGATGGTGTTGATTACGTTGTAAAGAAATATAAAGATATTTAT | |
| | GGTAAAGATGGAGAATCATTAAAGAAGTTATTTGATGATAAAAAAGAA | |
| | CAAACCCTTTTAATGGCTAGGAACGATATTCAAACATTTGAACTATTTA | |
| | AGAAAATATATGAAAACTATAAGAATGAAAAAAATCCATTCGCTAAATA | |
| | TAAGGATGAATTTGGACCAATAAAGAAATATTGTAAAGATGGAAAAGG | |
| | ACCTGAAATTTACCAGGTTAAATATATTTCTGACAAATTAGGTAATCAT | |
| | TTAGATATTTCGGATAAATATAACGTAGAAAATGGTAATAAAAAGGTTG | |
| | TTTTACTTCAAATTTCACCATATAGAACGGATGTGTATCAATCTAAAGA | |
| | AACTGGACTTTATAATTTTATTACAATAAGGAGATATCATATTTATCAG | |
| | GAAAATGATATGAATATTATAAAGCCTGAATTGTATCAAAAATTATTGT | |
| | CGGATAAAAAAATCGATGAAACTTATGAATTTAAATTTTCATTAAATAG | |
| | AAACGATATTATTAATTTAGTTACAATAGATAATCAAGCTGATCATATT | |
| | GATGAGATGCTCTATAGATTTATTGGAACTAATAATGATAAGACAAATA | |
| | AAATTGAGATTAGATATATTGAAAAAATGACATTAAAACAAATTATGGT | |
| | TTCAATTGGTAAGAAAATTGTTAAATTTGAAAAATATAATGTTAGTCCA | |
| | ACAGGTAAAAAAGCCAAAGCTGAAAAAGAAGTATTGAAATTAAAGTGG | |
| | AAGTAG | |
| ABSE Type II | GGCTACATCCTGGGCCTGGACCTGGGCATCACCAGCGTGGGATTTG | |
| Cas coding | GCATCATCGAAGAGGATACCTACAAAATCATCAAATATGGCGTCAGAC | 11 |
| sequence (nt) | TGTTTGATGAGGGAACAGCCGACAATAACCTGACCAGACGGCAGAAC | |
| codon | AGAAGCAGCCGTAGACTGAAGAGCAGACGGAAGAATAGAATCAACG | |
| optimized | CAATCAAGTACCTCCTCCTTAATAACAATGTGATCCCTAACGTGAACT | |
| | TTGATATCCTGGACAACGTGTACGAACTGAGAGTGAAGGGCCTGACC | |
| | CAGAAGCTGACCAACCTGGAACTGGCCAACGTCCTGGTGAACCTCG | |
| | CTAAAAGAAGGGGCTCTAGCCTCGACGTTGCCGTGGACGAGAACGA | |
| | CAACGAGAGCGTGGTGGCCGCCAGCGCCCTGACCAAGAACACCGCT | |
| | GAACTGAAGAAGAACAACCTGTACATTTGTCAGGCCCAGCTGGCCAA | |
| | GATAAAGAGCGGCGTGAAACTGAGAAACCATGATAACATCTACAAGT | |
| | CTGACGACTACGAGAAGGAACTGAGACAGATCCTGTCTAACCAAGAC | |
| | CTGTCCAACGAACTGAACGACAAGATCGTGGAAATCATCATGAGAAG | |
| | AAGAAATTTCAGCGAGGGCCCCGGCTCTTACGAGTTCCCTACCAAGT | |
| | ACGGCAGCTACAGAGTGGATTACGTGGACGGCATCAAGACCATCAAG | |
| | CACGTGAATCTGATCGAGGAAATGCGGGGCAAATGCAGCATCTTCAG | |
| | AAACGAGCCTAGAATCGCTAAGTGCAGTTTTGAGGCCTGCCTGTTCA | |
| | ATTTGCTGAACGATCTGAACAACCTGACAATTAACGGCAACGGCAAG | |
| | ATCACAAAGGAACAGAAGCTGCAGATCATTAACGATTATGTGATCCCT | |
| | AAGGGAAAGATCACCATACCCCAGCTGGTGAAGTATCTGGGCGTGGA | |
| | AGAGAAAGACATCACCGGTTTCAGAAAGAACAAATCAGAGAAGAACA | |
| | TCATCACAACATTCGATACATACAGCAAGATGATAAATTTCCTGCCAA | |
| | AGGACATCCTCAACGTGACCGCCTACGTTGATTCCATCATCGAGCTG | |
| | CTGACCAAAACCGCCGTGGTTGAGGAAAGAATCGAAGAGATCAAGAA | |
| | ACTGAATCTCGGACTGAACGACGACATCATCAACCAGCTGGCTAATA | |
| | TGACCAAGGTGAACGGCTACCACTCTCTGAGCAAGAAGGCTATGGAC | |
| | ATCATCATCCCTGAGATGATCGAGACAGATAAAAACCAGATGCAAATC | |
| | ATCCACGACAACAACCTGGACAAGAACGACTGCCAGGAGAAGGGCG | |
| | CCAAGATCAAGTTCGACAGCAGCGCCATCCTGTCTCCTGTGACCAAA | |
| | AGGGTGCACATGCAGGCCATCCAGGTGGTGAACGAGCTCCGCAAGG | |
| | AATACGGAGAGTTCGACAGCATCGTGATCGAAACCACCCGGGCCAA | |
| | GAATAGCTCCGACGAGAAGAAGGATATTCAGGAGGATCAGAAGCGG | |
| | CTGGAAGAGCAGAAAAGAAAGAGCGACGAACTGCTGATCGACCTGG | |
| | ACAAAAACCCCGATAATTACAACACCCTGACAAAGCTGAAACTGAGAC | |
| | TGTACAAGGAGCAGGAGGGCAAGACCATCTACGCCGGCCTGCCTAT | |
| | CGACCTGGATCTACTGCTGAATGATCCTACCGCCTACCAGATCGAGC | |
| | ACATCATCCCCTACGCCGTGTCTTTTGACAACAGCATGAACAACAAGT | |
| | CCCTGGCCAGCGCCAAGGAGAACCAAGACAAGGGAAAGAGAACACC | |
| | TTGGCAGTACTTCTCTAGCGGCATGGTCAGCGAGTGCGAGGGCCCT | |
| | ATCAACGACTGGGAATCTTTCGAGGCCTTCGTGAACAGCCTGAAGAT | |
| | ATCTAAGAACAAGAAGAACAACCTGCTGAATCAGGAGGACGTCACAA | |
| | AGTTTGATAATATGAAGGCTTTCGTGGCCCGGAACCTGAGCGACACC | |
| | TCTTACGACGTGCGGACCGTTATGAACACCATGCAACAGTACTTTAAC | |
| | GATAACAATATCAATACCAATGTGTTCACCGTGAAAGGCAGACTGACA | |
| | AACATATTCCGAAGCCGGGTGGGCCTGAAAAAGGACAGAGATTATTT | |
| | CATCCACCACGCCGTGGATGCCCTGATCATCGCCGGAAGCAAGAATC | |
| | AGTCTGTGTTCAACAACGCCTACCTGCTGAGCTCTACCAAGGACGGC | |
| | ATCCTGATCAAGAAAACCGGCGAGGTGTTCGACTACTCACAGGACCC | |
| | CTTCGACGACCCCAGATTCCTGAACTACATCAGCTCCCTGAAAAAGAT | |
| | CGAAGGAAAGCCCGAGGATTTCAGCTACAAGGTGGATAAGAAAACCA | |
| | ATCGGCAATTTGCCGACCAGACCATCTACAGCACCCGGAATTACGAC | |
| | GGCGTGGACTACGTGGTGAAGAAATACAAGGATATCTACGGCAAGGA | |
| | CGGCGAGAGCCTGAAAAAGCTGTTCGACGACAAGAAAGAACAGACC | |
| | CTGCTGATGGCCAGAAACGACATCCAGACCTTCGAGCTGTTCAAGAA | |
| | GATCTACGAGAACTACAAGAACGAGAAGAATCCCTTCGCCAAGTATA | |
| | AGGATGAGTTCGGCCCAATCAAGAAGTACTGCAAGGACGGCAAGGG | |
| | CCCTGAGATCTACCAAGTGAAGTATATCAGCGACAAGCTGGGAAACC | |
| | ACCTGGACATCAGCGACAAGTACAACGTGGAAAACGGCAATAAGAAG | |
| | GTGGTCCTGCTCCAGATCTCCCCTTATCGGACAGACGTGTATCAGAG | |
| | CAAGGAAACCGGACTGTACAACTTCATCACTATTCGGCGCTACCACA | |
| | TCTACCAGGAGAACGACATGAACATTATTAAACCTGAACTGTACCAAA | |
| | AGCTGCTGTCTGACAAGAAGATCGACGAGACATACGAGTTCAAGTTC | |
| | AGCCTCAATAGAAACGATATCATTAACCTGGTGACCATTGACAACCAG | |
| | GCGGACCACATCGATGAAATGCTGTACAGATTCATCGGCACAAACAA | |
| | CGACAAAACAAACAAGATCGAGATCCGGTACATCGAAAAGATGACAC | |
| | TGAAACAGATCATGGTGTCCATCGGCAAGAAGATCGTCAAATTCGAA | |
| | AAGTACAACGTGTCCCCAACAGGCAAGAAGGCCAAGGCCGAGAAGG | |
| | AAGTGCTGAAGCTGAAATGGAAG | |
| ABSE Type II | ATGATCCCCAATCCCCTGCTGGGCCTGGACAAGCGGACTGCCGACG | 12 |
| Cas | GCAGTGAGTTCGAGTCTCCCAAGAAAAAGAGGAAAGTGggatccGGCT | |
| mammalian | ACATCCTGGGCCTGGACCTGGGCATCACCAGCGTGGGATTTGGCAT | |
| expression | CATCGAAGAGGATACCTACAAAATCATCAAATATGGCGTCAGACTGTT | |
| construct | TGATGAGGGAACAGCCGACAATAACCTGACCAGACGGCAGAACAGA | |
| (includes N- | AGCAGCCGTAGACTGAAGAGCAGACGGAAGAATAGAATCAACGCAAT | |
| terminal SV5 | CAAGTACCTCCTCCTTAATAACAATGTGATCCCTAACGTGAACTTTGA | |
| tag and NLS | TATCCTGGACAACGTGTACGAACTGAGAGTGAAGGGCCTGACCCAGA | |
| and C- | AGCTGACCAACCTGGAACTGGCCAACGTCCTGGTGAACCTCGCTAAA | |
| terminal NLS) | AGAAGGGGCTCTAGCCTCGACGTTGCCGTGGACGAGAACGACAACG | |
| (nt) | AGAGCGTGGTGGCCGCCAGCGCCCTGACCAAGAACACCGCTGAACT | |
| | GAAGAAGAACAACCTGTACATTTGTCAGGCCCAGCTGGCCAAGATAA | |
| | AGAGCGGCGTGAAACTGAGAAACCATGATAACATCTACAAGTCTGAC | |
| | GACTACGAGAAGGAACTGAGACAGATCCTGTCTAACCAAGACCTGTC | |
| | CAACGAACTGAACGACAAGATCGTGGAAATCATCATGAGAAGAAGAA | |
| | ATTTCAGCGAGGGCCCCGGCTCTTACGAGTTCCCTACCAAGTACGGC | |
| | AGCTACAGAGTGGATTACGTGGACGGCATCAAGACCATCAAGCACGT | |
| | GAATCTGATCGAGGAAATGCGGGGCAAATGCAGCATCTTCAGAAACG | |
| | AGCCTAGAATCGCTAAGTGCAGTTTTGAGGCCTGCCTGTTCAATTTGC | |
| | TGAACGATCTGAACAACCTGACAATTAACGGCAACGGCAAGATCACA | |
| | AAGGAACAGAAGCTGCAGATCATTAACGATTATGTGATCCCTAAGGG | |
| | AAAGATCACCATACCCCAGCTGGTGAAGTATCTGGGCGTGGAAGAGA | |
| | AAGACATCACCGGTTTCAGAAAGAACAAATCAGAGAAGAACATCATCA | |
| | CAACATTCGATACATACAGCAAGATGATAAATTTCCTGCCAAAGGACA | |
| | TCCTCAACGTGACCGCCTACGTTGATTCCATCATCGAGCTGCTGACC | |
| | AAAACCGCCGTGGTTGAGGAAAGAATCGAAGAGATCAAGAAACTGAA | |
| | TCTCGGACTGAACGACGACATCATCAACCAGCTGGCTAATATGACCA | |
| | AGGTGAACGGCTACCACTCTCTGAGCAAGAAGGCTATGGACATCATC | |
| | ATCCCTGAGATGATCGAGACAGATAAAAACCAGATGCAAATCATCCAC | |
| | GACAACAACCTGGACAAGAACGACTGCCAGGAGAAGGGCGCCAAGA | |
| | TCAAGTTCGACAGCAGCGCCATCCTGTCTCCTGTGACCAAAAGGGTG | |
| | CACATGCAGGCCATCCAGGTGGTGAACGAGCTCCGCAAGGAATACG | |
| | GAGAGTTCGACAGCATCGTGATCGAAACCACCCGGGCCAAGAATAG | |
| | CTCCGACGAGAAGAAGGATATTCAGGAGGATCAGAAGCGGCTGGAA | |
| | GAGCAGAAAAGAAAGAGCGACGAACTGCTGATCGACCTGGACAAAAA | |
| | CCCCGATAATTACAACACCCTGACAAAGCTGAAACTGAGACTGTACAA | |
| | GGAGCAGGAGGGCAAGACCATCTACGCCGGCCTGCCTATCGACCTG | |
| | GATCTACTGCTGAATGATCCTACCGCCTACCAGATCGAGCACATCAT | |
| | CCCCTACGCCGTGTCTTTTGACAACAGCATGAACAACAAGTCCCTGG | |
| | CCAGCGCCAAGGAGAACCAAGACAAGGGAAAGAGAACACCTTGGCA | |
| | GTACTTCTCTAGCGGCATGGTCAGCGAGTGCGAGGGCCCTATCAAC | |
| | GACTGGGAATCTTTCGAGGCCTTCGTGAACAGCCTGAAGATATCTAA | |
| | GAACAAGAAGAACAACCTGCTGAATCAGGAGGACGTCACAAAGTTTG | |
| | ATAATATGAAGGCTTTCGTGGCCCGGAACCTGAGCGACACCTCTTAC | |
| | GACGTGCGGACCGTTATGAACACCATGCAACAGTACTTTAACGATAA | |
| | CAATATCAATACCAATGTGTTCACCGTGAAAGGCAGACTGACAAACAT | |
| | ATTCCGAAGCCGGGTGGGCCTGAAAAAGGACAGAGATTATTTCATCC | |
| | ACCACGCCGTGGATGCCCTGATCATCGCCGGAAGCAAGAATCAGTCT | |
| | GTGTTCAACAACGCCTACCTGCTGAGCTCTACCAAGGACGGCATCCT | |
| | GATCAAGAAAACCGGCGAGGTGTTCGACTACTCACAGGACCCCTTCG | |
| | ACGACCCCAGATTCCTGAACTACATCAGCTCCCTGAAAAAGATCGAA | |
| | GGAAAGCCCGAGGATTTCAGCTACAAGGTGGATAAGAAAACCAATCG | |
| | GCAATTTGCCGACCAGACCATCTACAGCACCCGGAATTACGACGGCG | |
| | TGGACTACGTGGTGAAGAAATACAAGGATATCTACGGCAAGGACGGC | |
| | GAGAGCCTGAAAAAGCTGTTCGACGACAAGAAAGAACAGACCCTGCT | |
| | GATGGCCAGAAACGACATCCAGACCTTCGAGCTGTTCAAGAAGATCT | |
| | ACGAGAACTACAAGAACGAGAAGAATCCCTTCGCCAAGTATAAGGAT | |
| | GAGTTCGGCCCAATCAAGAAGTACTGCAAGGACGGCAAGGGCCCTG | |
| | AGATCTACCAAGTGAAGTATATCAGCGACAAGCTGGGAAACCACCTG | |
| | GACATCAGCGACAAGTACAACGTGGAAAACGGCAATAAGAAGGTGGT | |
| | CCTGCTCCAGATCTCCCCTTATCGGACAGACGTGTATCAGAGCAAGG | |
| | AAACCGGACTGTACAACTTCATCACTATTCGGCGCTACCACATCTACC | |
| | AGGAGAACGACATGAACATTATTAAACCTGAACTGTACCAAAAGCTGC | |
| | TGTCTGACAAGAAGATCGACGAGACATACGAGTTCAAGTTCAGCCTC | |
| | AATAGAAACGATATCATTAACCTGGTGACCATTGACAACCAGGCGGA | |
| | CCACATCGATGAAATGCTGTACAGATTCATCGGCACAAACAACGACA | |
| | AAACAAACAAGATCGAGATCCGGTACATCGAAAAGATGACACTGAAA | |
| | CAGATCATGGTGTCCATCGGCAAGAAGATCGTCAAATTCGAAAAGTA | |
| | CAACGTGTCCCCAACAGGCAAGAAGGCCAAGGCCGAGAAGGAAGTG | |
| | CTGAAGCTGAAATGGAAGtctagaAAGCGGACAGCAGACGGCTCCGAA | |
| | TTTGAAAGCCCTAAGAAAAAGAGAAAGGTGTGA | |

In some embodiments an ABSE Type II Cas protein comprises an amino acid sequence of SEQ ID NO:7, SEQ ID NO:8, or SEQ ID NO:9. In some embodiments, an ABSE Type II Cas protein has nickase activity, for example resulting from one or more amino acid substitutions relative to the sequence of SEQ ID NO:7, SEQ ID NO:8, or SEQ ID NO:9. In some embodiments, the one or more amino acid substitutions providing nickase activity is a D8A substitution, wherein the position of the D8A substitution is defined with respect to the amino acid numbering of SEQ ID NO:8. In some embodiments, the one or more amino acid substitutions providing nickase activity is a N587A substitution, wherein the position of the N587A substitution is defined with respect to the amino acid numbering of SEQ ID NO:8. In some embodiments, an ABSE Type II Cas protein is catalytically inactive, for example due to both a D8A substitution and a N587A substitution, where the positions of the substitutions are defined with respect to the amino acid numbering of SEQ ID NO:8.

### 6.2.1.3. AIXM Type II Cas Proteins

In one aspect, the disclosure provides AIXM Type II Cas proteins. AIXM Type II Cas proteins can be further classified as Type IIA Cas proteins. The AIXM Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:13. In some embodiments, the AIXM Type II Cas proteins comprise an amino acid sequence that is at least 95%, at least 96%, at least 97%, at least 98%, or at least 99% identical to SEQ ID NO:13. In some embodiments, an AIXM Type II Cas protein comprises an amino acid sequence that is identical to SEQ ID NO:13.

Exemplary AIXM Type II Cas protein sequences and nucleotide sequences encoding exemplary AIXM Type II Cas proteins are set forth in Table 1C.

TABLE 1C
AIXM Type II Cas Sequences

| | | SEQ ID |
|---|---|---|
| Name | Sequence | NO: |
| AIXM Type II | AKYILGLDMGITSVGWGLIDYDSGKIVDKGVRLFKEATAEDNLNRRTHRG | 13 |
| Cas amino | SRRLKRRRQQRIIELKRLLKKENILTDDYTPLNNVYYLRCKGLNEKLTNAE | |
| acid | LVSVLLHIAKIRGSSLEVVEDDEKKANEDKSSKANLSANEVKLRTSKEEIC | |
| sequence | EKQYKDLIENGKVRGINNIYKSSSYESELKKILANQGLSKEVNDKIINIIFRR | |
| (without N- | RDFNEGPGSMESPTIYGRFIPHENGEVEVIHSMIEKMQGKCSIYQNEPRA | |
| terminal | AKMSFTADLFNFLNDLNNLEIQRADNYDNYITPDEKKLIINEYIIKKGKITIQ | |
| valine) | KLCDVLQVKKENITGFRVNKGEPILTEFKGYQKVLKALNRIIDTEIVDAVID | |
| | VLTMHKDVEKRKEEIKKINNTEFSEEDIVKLSEITGIQQYHSLSYKAMKVII | |
| | PELYENPENQMEVITRLNLVKTSIDDLKGRTTIPSYSNRIYSPVAKRVHNE | |
| | TIKVINAVIKKYGDLDSIVIEMARDRNTAEERKNIEQAQKRNQENREKAKEI | |
| | LNVNYSFSKLDSKLVQKIMLYNQQDGKSMYSGKPINLNVLISDPNAYEID | |
| | HIIPLSISLDDSIANKVLVYRSENQQKLNKTPLQYLRSGNSNGWSVDEFR | |
| | EVVIKMYNDKKISLKKLQNLLCEKDITKQDVRKEFIERNLVDTRYASRVVL | |
| | GLLKDYFKANNKNTKVFTIRGQITSQFRKSRNLVKSRDCFYHHIVDALIIAY | |
| | FRKFNYINELMQYNFNYKGKIDEETGELIQYNDTLFSTKDDDFTIQLRKLN | |
| | DLNKYSEDIKISHKVDRKPNRKFSDETIYSAIKIGNDYMKISKYKDIYGEEG | |
| | KKFAKKIKDNLQKENELDIFLMKKVDPETFNLLVSIVKNTICGEKENPFVK | |
| | YCEENNIDFIRKKSKNGNGPIIRSIRYYDGKVNSYQDISQKYSNLSDDKKV | |
| | VRLHISPYRTDFYLTKEGNYKFLTIKYANIKENHNGYYIPKEVYETLKKSKK | |
| | INDSDEFLFSLYRNDYLRITTNDPTENAPMIYRFVGTKKDLNNVIEVKPIYC | |
| | DKIDKPQIEKTIGKKVTKIEKFDVDVLGNKIAKKRDKCLQLEL | |
| AIXM Type II | VAKYILGLDMGITSVGWGLIDYDSGKIVDKGVRLFKEATAEDNLNRRTHR | 14 |
| Cas amino | GSRRLKRRRQQRIIELKRLLKKENILTDDYTPLNNVYYLRCKGLNEKLTNA | |
| acid | ELVSVLLHIAKIRGSSLEVVEDDEKKANEDKSSKANLSANEVKLRTSKEEI | |
| sequence | CEKQYKDLIENGKVRGINNIYKSSSYESELKKILANQGLSKEVNDKIINIIFR | |
| | RRDFNEGPGSMESPTIYGRFIPHENGEVEVIHSMIEKMQGKCSIYQNEPR | |
| | AAKMSFTADLFNFLNDLNNLEIQRADNYDNYITPDEKKLIINEYIIKKGKITI | |
| | QKLCDVLQVKKENITGFRVNKGEPILTEFKGYQKVLKALNRIIDTEIVDAVI | |
| | DVLTMHKDVEKRKEEIKKINNTEFSEEDIVKLSEITGIQQYHSLSYKAMKVI | |
| | IPELYENPENQMEVITRLNLVKTSIDDLKGRTTIPSYSNRIYSPVAKRVHNE | |
| | TIKVINAVIKKYGDLDSIVIEMARDRNTAEERKNIEQAQKRNQENREKAKEI | |
| | LNVNYSFSKLDSKLVQKIMLYNQQDGKSMYSGKPINLNVLISDPNAYEID | |
| | HIIPLSISLDDSIANKVLVYRSENQQKLNKTPLQYLRSGNSNGWSVDEFR | |
| | EVVIKMYNDKKISLKKLQNLLCEKDITKQDVRKEFIERNLVDTRYASRVVL | |
| | GLLKDYFKANNKNTKVFTIRGQITSQFRKSRNLVKSRDCFYHHIVDALIIAY | |
| | FRKFNYINELMQYNFNYKGKIDEETGELIQYNDTLFSTKDDDFTIQLRKLN | |
| | DLNKYSEDIKISHKVDRKPNRKFSDETIYSAIKIGNDYMKISKYKDIYGEEG | |
| | KKFAKKIKDNLQKENELDIFLMKKVDPETFNLLVSIVKNTICGEKENPFVK | |
| | YCEENNIDFIRKKSKNGNGPIIRSIRYYDGKVNSYQDISQKYSNLSDDKKV | |
| | VRLHISPYRTDFYLTKEGNYKFLTIKYANIKENHNGYYIPKEVYETLKKSKK | |
| | INDSDEFLFSLYRNDYLRITTNDPTENAPMIYRFVGTKKDLNNVIEVKPIYC | |
| | DKIDKPQIEKTIGKKVTKIEKFDVDVLGNKIAKKRDKCLQLEL | |
| AIXM Type II | MIPNPLLGLDKRTADGSEFESPKKKRKVGSAKYILGLDMGITSVGWGLID | 15 |
| Cas | YDSGKIVDKGVRLFKEATAEDNLNRRTHRGSRRLKRRRQQRIIELKRLLK | |
| mammalian | KENILTDDYTPLNNVYYLRCKGLNEKLTNAELVSVLLHIAKIRGSSLEVVE | |
| expression | DDEKKANEDKSSKANLSANEVKLRTSKEEICEKQYKDLIENGKVRGINNI | |
| construct | YKSSSYESELKKILANQGLSKEVNDKIINIIFRRRDFNEGPGSMESPTIYGR | |
| (includes N- | FIPHENGEVEVIHSMIEKMQGKCSIYQNEPRAAKMSFTADLFNFLNDLNN | |
| terminal SV5 | LEIQRADNYDNYITPDEKKLIINEYIIKKGKITIQKLCDVLQVKKENITGFRVN | |
| tag and NLS | KGEPILTEFKGYQKVLKALNRIIDTEIVDAVIDVLTMHKDVEKRKEEIKKINN | |
| and C- | TEFSEEDIVKLSEITGIQQYHSLSYKAMKVIIPELYENPENQMEVITRLNLV | |
| terminal NLS) | KTSIDDLKGRTTIPSYSNRIYSPVAKRVHNETIKVINAVIKKYGDLDSIVIEM | |
| (aa) | ARDRNTAEERKNIEQAQKRNQENREKAKEILNVNYSFSKLDSKLVQKIML | |
| | YNQQDGKSMYSGKPINLNVLISDPNAYEIDHIIPLSISLDDSIANKVLVYRS | |
| | ENQQKLNKTPLQYLRSGNSNGWSVDEFREVVIKMYNDKKISLKKLQNLL | |
| | CEKDITKQDVRKEFIERNLVDTRYASRVVLGLLKDYFKANNKNTKVFTIRG | |
| | QITSQFRKSRNLVKSRDCFYHHIVDALIIAYFRKFNYINELMQYNFNYKGKI | |
| | DEETGELIQYNDTLFSTKDDDFTIQLRKLNDLNKYSEDIKISHKVDRKPNR | |
| | KFSDETIYSAIKIGNDYMKISKYKDIYGEEGKKFAKKIKDNLQKENELDIFL | |
| | MKKVDPETFNLLVSIVKNTICGEKENPFVKYCEENNIDFIRKKSKNGNGPII | |
| | RSIRYYDGKVNSYQDISQKYSNLSDDKKVVRLHISPYRTDFYLTKEGNYK | |
| | FLTIKYANIKENHNGYYIPKEVYETLKKSKKINDSDEFLFSLYRNDYLRITT | |
| | NDPTENAPMIYRFVGTKKDLNNVIEVKPIYCDKIDKPQIEKTIGKKVTKIEK | |
| | FDVDVLGNKIAKKRDKCLQLELSRKRTADGSEFESPKKKRKV | |
| AIXM Type II | GTGGCAAAATATATATTAGGACTAGATATGGGAATTACTTCGGTAGGT | 16 |
| Cas coding | TGGGGCCTAATTGACTATGATTCTGGAAAAATAGTTGATAAAGGCGTT | |
| sequence (nt) | AGACTTTTTAAAGAAGCAACTGCGGAAGATAATTTAAACAGACGTACT | |
| not codon | CATCGTGGTTCAAGAAGGTTAAAAAGACGTAGACAACAAAGAATAATT | |
| optimized | GAATTAAAACGATTATTAAAAAAAGAAAACATTCTTACAGATGATTATA | |
| | CTCCTTTAAATAATGTTTACTATCTTAGATGTAAAGGCTTAAACGAAAA | |
| | ATTAACAAATGCTGAACTTGTATCAGTATTATTACATATAGCAAAAATA | |
| | AGAGGAAGTTCCCTAGAAGTAGTTGAAGATGATGAAAAAAAGGCTAAT | |
| | GAGGATAAAAGTAGTAAAGCTAATTTATCAGCAAACGAAGTAAAATTA | |
| | AGAACTTCAAAAGAAGAAATTTGTGAAAAACAATATAAAGATCTAATCG | |
| | AAAATGGTAAAGTTCGTGGAATAAATAATATTTATAAAAGTTCTTCATA | |
| | TGAGTCAGAACTCAAAAAAATTTTAGCTAATCAAGGCTTGTCAAAAGA | |
| | AGTTAATGATAAGATAATAAATATAATTTTCAGAAGACGTGACTTTAAT | |
| | GAAGGACCAGGAAGCATGGAATCGCCTACTATATATGGTAGATTTATA | |
| | CCACATGAAAATGGTGAGGTAGAAGTTATACATAGTATGATTGAAAAA | |
| | ATGCAAGGGAAATGTTCCATATACCAAAACGAACCTCGTGCAGCAAA | |
| | AATGTCATTTACAGCTGATTTGTTTAATTTTTTAAATGATTTAAACAATT | |
| | TAGAAATACAGCGTGCTGATAACTATGATAATTATATTACCCCAGACG | |
| | AAAAAAAACTAATAATAAATGAGTATATCATAAAAAAAGGTAAAATTAC | |
| | AATTCAAAAATTATGCGATGTACTACAGGTAAAAAAAGAAAATATTACT | |
| | GGCTTTAGAGTTAACAAAGGTGAACCAATACTCACTGAATTTAAAGGT | |
| | TATCAAAAAGTATTAAAAGCTTTAAATAGAATAATTGATACTGAAATTG | |
| | TTGATGCGGTTATTGATGTTTTAACAATGCATAAAGATGTAGAAAAAA | |
| | GAAAAGAAGAAATTAAAAAAATAAACAATACAGAGTTTAGCGAAGAAG | |
| | ACATTGTAAAATTATCTGAAATAACCGGTATACAACAGTATCATTCATT | |
| | GTCTTATAAAGCAATGAAAGTAATAATACCTGAATTATATGAAAATCCT | |
| | GAAAATCAAATGGAAGTAATTACAAGATTAAATCTAGTGAAAACTTCAA | |
| | TAGATGATTTGAAAGGAAGAACAACAATACCTTCATATAGCAACAGAA | |
| | TATATTCCCCTGTAGCTAAACGTGTACATAATGAGACAATCAAAGTAA | |
| | TAAATGCGGTAATAAAAAAATATGGAGATTTAGATAGTATTGTTATAGA | |
| | AATGGCAAGGGATAGAAATACTGCCGAAGAAAGAAAAAATATTGAACA | |
| | AGCCCAAAAACGAAATCAAGAAAATAGAGAAAAAGCAAAAGAGATACT | |
| | AAATGTTAATTATAGTTTTTCGAAGCTTGATTCAAAATTAGTTCAAAAG | |
| | ATCATGCTTTATAATCAACAAGATGGTAAAAGTATGTATAGTGGAAAA | |
| | CCAATAAACTTAAATGTTTTGATAAGTGACCCTAATGCATACGAGATA | |
| | GATCATATCATACCACTATCAATTTCGCTTGATGATTCAATTGCCAATA | |
| | AAGTTCTGGTGTATAGAAGTGAAAACCAACAAAAATTAAATAAAACAC | |
| | CACTTCAATACTTACGTAGTGGTAATTCAAATGGTTGGAGCGTCGACG | |
| | AATTCCGTGAAGTTGTAATAAAAATGTATAATGATAAAAAAATATCACT | |
| | AAAAAAATTACAAAACTTATTATGTGAAAAAGATATAACAAAACAAGAT | |
| | GTAAGAAAAGAATTTATTGAAAGAAATTTAGTTGACACAAGATATGCAA | |
| | GTAGGGTAGTTTTGGGACTTCTAAAAGATTATTTCAAAGCCAACAATA | |
| | AAAATACTAAAGTATTTACAATAAGAGGCCAAATAACATCACAATTCAG | |
| | AAAAAGTAGAAACTTGGTGAAAAGTAGAGATTGCTTTTATCATCATATA | |
| | GTAGATGCATTAATAATTGCATATTTTAGAAAATTCAATTATATTAATGA | |
| | ATTAATGCAATACAATTTTAATTATAAAGGGAAAATTGATGAAGAAACA | |
| | GGAGAATTAATCCAATATAATGATACGTTGTTTTCAACGAAGGATGAT | |
| | GATTTCACAATACAATTAAGAAAACTAAATGACCTTAATAAATATAGTG | |
| | AAGATATCAAAATTTCGCATAAAGTTGATAGAAAGCCTAATAGAAAGTT | |
| | TAGTGATGAAACAATCTATTCTGCGATAAAGATCGGCAACGACTATAT | |
| | GAAAATTTCGAAATATAAAGATATTTACGGTGAAGAAGGCAAAAAATTT | |
| | GCAAAAAAAATTAAAGATAATCTTCAAAAGGAAAATGAATTAGATATAT | |
| | TCTTAATGAAAAAAGTTGATCCAGAAACTTTCAATTTATTAGTTAGTAT | |
| | TGTTAAGAATACAATATGTGGTGAAAAAGAAAATCCATTTGTTAAATAC | |
| | TGTGAAGAAAACAATATAGATTTTATTAGAAAAAAATCTAAAAATGGCA | |
| | ATGGTCCGATTATTAGGTCTATTAGATATTATGATGGTAAAGTAAATAG | |
| | TTACCAGGATATCTCACAAAAATATTCAAACTTATCAGATGACAAAAAA | |
| | GTGGTGCGTTTACACATTTCGCCGTATAGAACTGATTTTTATTTAACTA | |
| | AAGAAGGAAACTATAAATTCCTAACAATTAAATATGCTAATATCAAAGA | |
| | AAACCATAATGGATATTATATACCAAAAGAAGTTTACGAAACATTAAAA | |
| | AAATCAAAGAAAATAAATGATAGTGATGAATTTTTATTTTCGCTATATC | |
| | GAAATGATTACCTAAGAATTACAACAAATGATCCTACTGAAAATGCTC | |
| | CTATGATTTATCGATTTGTAGGAACAAAAAAAGATCTAAATAACGTTAT | |
| | AGAAGTAAAACCAATTTATTGTGATAAGATAGATAAACCACAAATTGA | |
| | GAAAACCATAGGTAAAAAAGTAACTAAAATTGAAAAGTTTGATGTTGAT | |
| | GTACTAGGTAATAAAATTGCTAAAAAAAGAGATAAATGCTTGCAATTA | |
| | GAGCTATAG | |
| AIXM Type II | GCCAAGTACATCCTGGGACTGGATATGGGCATCACCAGCGTGGGCT | 17 |
| Cas coding | GGGGCCTGATCGACTACGACAGCGGAAAGATCGTGGATAAGGGCGT | |
| sequence (nt) | CCGGCTGTTTAAAGAGGCAACCGCCGAGGATAATCTGAACAGAAGGA | |
| codon | CACACAGAGGCAGCCGGCGGCTGAAGCGCAGACGGCAGCAAAGAAT | |
| optimized | CATCGAGCTGAAAAGACTGCTGAAGAAGGAAAACATCCTGACAGATG | |
| | ACTACACCCCTCTGAATAATGTGTACTACCTGAGATGCAAAGGCCTGA | |
| | ACGAGAAGCTGACCAACGCTGAACTGGTGTCTGTGCTGCTGCACATC | |
| | GCCAAGATACGGGGCAGCAGCCTTGAGGTCGTGGAAGATGACGAGA | |
| | AGAAGGCGAATGAGGACAAGAGCTCCAAGGCCAATCTGAGCGCTAA | |
| | CGAGGTGAAGCTCAGAACATCTAAGGAGGAGATCTGCGAGAAGCAG | |
| | TACAAGGACCTAATAGAGAACGGAAAGGTCCGGGGCATCAATAACAT | |
| | CTATAAGTCTAGCTCCTACGAATCAGAGCTGAAGAAAATCCTGGCCAA | |
| | TCAGGGCCTGAGCAAAGAGGTGAACGACAAGATCATCAACATCATTT | |
| | TCCGCCGGCGAGACTTCAACGAAGGCCCCGGGAGCATGGAAAGCCC | |
| | TACAATCTACGGCAGATTCATCCCTCACGAGAACGGCGAGGTTGAAG | |
| | TGATCCACAGCATGATCGAAAAGATGCAGGGCAAATGCAGCATCTAC | |
| | CAGAACGAGCCTAGAGCCGCTAAGATGAGCTTCACAGCCGACCTGTT | |
| | CAATTTCCTGAACGACCTGAACAACCTGGAAATCCAGAGAGCCGATA | |
| | ACTACGACAACTACATTACCCCTGATGAGAAAAAGCTGATCATCAACG | |
| | AGTACATCATCAAGAAAGGCAAGATCACCATTCAGAAACTCTGCGAC | |
| | GTGCTGCAAGTGAAGAAGGAAAATATCACCGGCTTCCGGGTGAACAA | |
| | GGGCGAACCTATCCTGACAGAGTTTAAGGGCTACCAAAAGGTGCTGA | |
| | AGGCCCTGAACCGGATCATCGACACCGAGATCGTGGACGCCGTGAT | |
| | CGACGTGCTGACCATGCACAAAGACGTGGAAAAGAGAAAGGAGGAG | |
| | ATCAAAAAGATCAACAACACCGAGTTTTCTGAAGAGGATATCGTGAAG | |
| | CTGTCTGAAATCACTGGAATCCAGCAGTACCATTCTCTGAGCTACAAA | |
| | GCCATGAAAGTGATCATCCCTGAGCTGTACGAGAACCCAGAGAACCA | |
| | GATGGAAGTGATCACCAGACTGAACCTGGTTAAGACAAGCATCGACG | |
| | ACCTGAAGGGAAGAACCACCATTCCATCTTACAGCAATAGAATCTATA | |
| | GCCCCGTGGCCAAGAGGGTCCATAATGAAACTATTAAGGTGATCAAC | |
| | GCCGTGATCAAGAAATACGGCGACCTGGATTCCATTGTGATCGAAAT | |
| | GGCCAGAGATAGAAACACCGCCGAAGAGCGGAAGAACATCGAACAG | |
| | GCCCAGAAGCGGAATCAAGAGAATAGGGAAAAGGCTAAAGAAATCCT | |
| | GAACGTGAACTACAGCTTTAGCAAGCTGGACAGCAAGCTGGTCCAGA | |
| | AAATTATGCTGTACAACCAGCAGGACGGAAAGAGCATGTACAGCGGC | |
| | AAGCCCATCAACCTGAACGTGCTGATCTCCGATCCCAATGCTTACGA | |
| | GATCGACCACATCATCCCCTTGTCCATTAGTCTGGATGATAGCATCGC | |
| | CAACAAAGTGCTGGTGTACAGAAGCGAGAACCAGCAGAAGCTGAACA | |
| | AGACCCCTCTGCAGTACCTGAGATCTGGCAATAGCAACGGCTGGAGC | |
| | GTGGACGAGTTCAGAGAGGTGGTGATCAAGATGTACAACGACAAAAA | |
| | AATCTCTCTGAAGAAGCTGCAGAACCTGCTCTGTGAAAAGGACATTAC | |
| | CAAGCAGGACGTGCGGAAAGAGTTCATCGAACGGAACCTGGTGGAC | |
| | ACCCGGTACGCCAGCAGAGTGGTGCTGGGCCTGCTGAAGGACTACT | |
| | TCAAGGCCAACAACAAGAACACCAAGGTCTTTACCATCAGAGGCCAG | |
| | ATCACCAGCCAGTTCAGAAAGAGCAGAAACCTGGTGAAATCTAGGGA | |
| | TTGTTTCTACCACCACATCGTGGACGCCCTGATCATCGCCTACTTCAG | |
| | AAAGTTTAACTATATCAATGAACTGATGCAGTACAACTTCAACTACAAG | |
| | GGAAAGATTGACGAGGAGACAGGCGAGCTGATCCAGTACAATGACA | |
| | CCCTGTTCAGCACCAAGGACGATGACTTTACCATCCAACTGAGAAAG | |
| | CTGAACGACCTGAACAAGTACAGCGAGGATATCAAGATCAGCCACAA | |
| | GGTGGATAGAAAGCCTAACCGGAAATTCTCCGACGAGACAATCTACA | |
| | GCGCCATCAAAATCGGAAACGACTACATGAAGATATCCAAGTATAAG | |
| | GATATCTATGGAGAGGAAGGCAAAAAGTTCGCCAAGAAAATCAAGGA | |
| | CAACCTGCAAAAAGAAAATGAGCTGGATATCTTCCTGATGAAGAAAGT | |
| | GGACCCCGAGACATTCAACTTACTAGTGAGCATCGTGAAGAACACAA | |
| | TCTGCGGCGAGAAGGAGAACCCCTTCGTAAAATATTGCGAGGAAAAT | |
| | AATATCGACTTCATCAGAAAAAAGTCTAAGAACGGCAATGGCCCTATC | |
| | ATCCGGTCCATCAGATACTACGATGGCAAAGTTAACAGCTACCAGGA | |
| | CATCAGCCAGAAGTACAGCAACCTGAGCGATGACAAGAAGGTTGTGC | |
| | GGCTGCACATCTCTCCTTACCGGACCGACTTCTACCTGACCAAGGAA | |
| | GGCAACTACAAGTTCCTGACCATCAAGTACGCCAACATCAAGGAGAA | |
| | CCACAACGGCTACTATATCCCTAAGGAAGTGTACGAGACACTGAAGA | |
| | AGTCTAAGAAGATCAACGACAGCGACGAGTTCCTGTTCTCCCTGTAC | |
| | AGAAATGACTACCTGAGAATCACAACAAACGACCCTACCGAAAACGC | |
| | TCCTATGATCTACAGATTCGTGGGCACCAAGAAGGATCTGAATAACGT | |
| | GATCGAAGTGAAGCCCATTTACTGTGACAAGATCGACAAACCTCAGA | |
| | TCGAAAAGACCATCGGCAAAAAGGTGACCAAGATCGAGAAGTTCGAC | |
| | GTGGATGTTCTGGGGAACAAGATCGCCAAGAAGCGGGACAAGTGCC | |
| | TGCAGCTGGAATTG | |
| AIXM Type II | ATGATCCCCAATCCCCTGCTGGGCCTGGACAAGCGGACTGCCGACG | 18 |
| Cas coding | GCAGTGAGTTCGAGTCTCCCAAGAAAAAGAGGAAAGTGggatccGCCA | |
| sequence (nt) | AGTACATCCTGGGACTGGATATGGGCATCACCAGCGTGGGCTGGGG | |
| codon | CCTGATCGACTACGACAGCGGAAAGATCGTGGATAAGGGCGTCCGG | |
| optimized(inc | CTGTTTAAAGAGGCAACCGCCGAGGATAATCTGAACAGAAGGACACA | |
| luding V5-tag | CAGAGGCAGCCGGCGGCTGAAGCGCAGACGGCAGCAAAGAATCATC | |
| and N- and C- | GAGCTGAAAAGACTGCTGAAGAAGGAAAACATCCTGACAGATGACTA | |
| terminal NLS) | CACCCCTCTGAATAATGTGTACTACCTGAGATGCAAAGGCCTGAACG | |
| | AGAAGCTGACCAACGCTGAACTGGTGTCTGTGCTGCTGCACATCGCC | |
| | AAGATACGGGGCAGCAGCCTTGAGGTCGTGGAAGATGACGAGAAGA | |
| | AGGCGAATGAGGACAAGAGCTCCAAGGCCAATCTGAGCGCTAACGA | |
| | GGTGAAGCTCAGAACATCTAAGGAGGAGATCTGCGAGAAGCAGTACA | |
| | AGGACCTAATAGAGAACGGAAAGGTCCGGGGCATCAATAACATCTAT | |
| | AAGTCTAGCTCCTACGAATCAGAGCTGAAGAAAATCCTGGCCAATCA | |
| | GGGCCTGAGCAAAGAGGTGAACGACAAGATCATCAACATCATTTTCC | |
| | GCCGGCGAGACTTCAACGAAGGCCCCGGGAGCATGGAAAGCCCTAC | |
| | AATCTACGGCAGATTCATCCCTCACGAGAACGGCGAGGTTGAAGTGA | |
| | TCCACAGCATGATCGAAAAGATGCAGGGCAAATGCAGCATCTACCAG | |
| | AACGAGCCTAGAGCCGCTAAGATGAGCTTCACAGCCGACCTGTTCAA | |
| | TTTCCTGAACGACCTGAACAACCTGGAAATCCAGAGAGCCGATAACT | |
| | ACGACAACTACATTACCCCTGATGAGAAAAAGCTGATCATCAACGAGT | |
| | ACATCATCAAGAAAGGCAAGATCACCATTCAGAAACTCTGCGACGTG | |
| | CTGCAAGTGAAGAAGGAAAATATCACCGGCTTCCGGGTGAACAAGGG | |
| | CGAACCTATCCTGACAGAGTTTAAGGGCTACCAAAAGGTGCTGAAGG | |
| | CCCTGAACCGGATCATCGACACCGAGATCGTGGACGCCGTGATCGA | |
| | CGTGCTGACCATGCACAAAGACGTGGAAAAGAGAAAGGAGGAGATC | |
| | AAAAAGATCAACAACACCGAGTTTTCTGAAGAGGATATCGTGAAGCTG | |
| | TCTGAAATCACTGGAATCCAGCAGTACCATTCTCTGAGCTACAAAGCC | |
| | ATGAAAGTGATCATCCCTGAGCTGTACGAGAACCCAGAGAACCAGAT | |
| | GGAAGTGATCACCAGACTGAACCTGGTTAAGACAAGCATCGACGACC | |
| | TGAAGGGAAGAACCACCATTCCATCTTACAGCAATAGAATCTATAGCC | |
| | CCGTGGCCAAGAGGGTCCATAATGAAACTATTAAGGTGATCAACGCC | |
| | GTGATCAAGAAATACGGCGACCTGGATTCCATTGTGATCGAAATGGC | |
| | CAGAGATAGAAACACCGCCGAAGAGCGGAAGAACATCGAACAGGCC | |
| | CAGAAGCGGAATCAAGAGAATAGGGAAAAGGCTAAAGAAATCCTGAA | |
| | CGTGAACTACAGCTTTAGCAAGCTGGACAGCAAGCTGGTCCAGAAAA | |
| | TTATGCTGTACAACCAGCAGGACGGAAAGAGCATGTACAGCGGCAAG | |
| | CCCATCAACCTGAACGTGCTGATCTCCGATCCCAATGCTTACGAGAT | |
| | CGACCACATCATCCCCTTGTCCATTAGTCTGGATGATAGCATCGCCAA | |
| | CAAAGTGCTGGTGTACAGAAGCGAGAACCAGCAGAAGCTGAACAAGA | |
| | CCCCTCTGCAGTACCTGAGATCTGGCAATAGCAACGGCTGGAGCGT | |
| | GGACGAGTTCAGAGAGGTGGTGATCAAGATGTACAACGACAAAAAAA | |
| | TCTCTCTGAAGAAGCTGCAGAACCTGCTCTGTGAAAAGGACATTACC | |
| | AAGCAGGACGTGCGGAAAGAGTTCATCGAACGGAACCTGGTGGACA | |
| | CCCGGTACGCCAGCAGAGTGGTGCTGGGCCTGCTGAAGGACTACTT | |
| | CAAGGCCAACAACAAGAACACCAAGGTCTTTACCATCAGAGGCCAGA | |
| | TCACCAGCCAGTTCAGAAAGAGCAGAAACCTGGTGAAATCTAGGGAT | |
| | TGTTTCTACCACCACATCGTGGACGCCCTGATCATCGCCTACTTCAGA | |
| | AAGTTTAACTATATCAATGAACTGATGCAGTACAACTTCAACTACAAG | |
| | GGAAAGATTGACGAGGAGACAGGCGAGCTGATCCAGTACAATGACA | |
| | CCCTGTTCAGCACCAAGGACGATGACTTTACCATCCAACTGAGAAAG | |
| | CTGAACGACCTGAACAAGTACAGCGAGGATATCAAGATCAGCCACAA | |
| | GGTGGATAGAAAGCCTAACCGGAAATTCTCCGACGAGACAATCTACA | |
| | GCGCCATCAAAATCGGAAACGACTACATGAAGATATCCAAGTATAAG | |
| | GATATCTATGGAGAGGAAGGCAAAAAGTTCGCCAAGAAAATCAAGGA | |
| | CAACCTGCAAAAAGAAAATGAGCTGGATATCTTCCTGATGAAGAAAGT | |
| | GGACCCCGAGACATTCAACTTACTAGTGAGCATCGTGAAGAACACAA | |
| | TCTGCGGCGAGAAGGAGAACCCCTTCGTAAAATATTGCGAGGAAAAT | |
| | AATATCGACTTCATCAGAAAAAAGTCTAAGAACGGCAATGGCCCTATC | |
| | ATCCGGTCCATCAGATACTACGATGGCAAAGTTAACAGCTACCAGGA | |
| | CATCAGCCAGAAGTACAGCAACCTGAGCGATGACAAGAAGGTTGTGC | |
| | GGCTGCACATCTCTCCTTACCGGACCGACTTCTACCTGACCAAGGAA | |
| | GGCAACTACAAGTTCCTGACCATCAAGTACGCCAACATCAAGGAGAA | |
| | CCACAACGGCTACTATATCCCTAAGGAAGTGTACGAGACACTGAAGA | |
| | AGTCTAAGAAGATCAACGACAGCGACGAGTTCCTGTTCTCCCTGTAC | |
| | AGAAATGACTACCTGAGAATCACAACAAACGACCCTACCGAAAACGC | |
| | TCCTATGATCTACAGATTCGTGGGCACCAAGAAGGATCTGAATAACGT | |
| | GATCGAAGTGAAGCCCATTTACTGTGACAAGATCGACAAACCTCAGA | |
| | TCGAAAAGACCATCGGCAAAAAGGTGACCAAGATCGAGAAGTTCGAC | |
| | GTGGATGTTCTGGGGAACAAGATCGCCAAGAAGCGGGACAAGTGCC | |
| | TGCAGCTGGAATTGtctagaAAGCGGACAGCAGACGGCTCCGAATTTGA | |
| | AAGCCCTAAGAAAAAGAGAAAGGTGTGA | |

In some embodiments an AIXM Type II Cas protein comprises an amino acid sequence of SEQ ID NO:13, SEQ ID NO:14, or SEQ ID NO:15. In some embodiments, an AIXM Type II Cas protein has nickase activity, for example resulting from one or more amino acid substitutions relative to the sequence of SEQ ID NO:13, SEQ ID NO:14, or SEQ ID NO:15. In some embodiments, the one or more amino acid substitutions providing nickase activity is a D9A substitution, wherein the position of the D9A substitution is defined with respect to the amino acid numbering of SEQ ID NO:14. In some embodiments, the one or more amino acid substitutions providing nickase activity is a N590A substitution, wherein the position of the N590A substitution is defined with respect to the amino acid numbering of SEQ ID NO:14. In some embodiments, an AIXM Type II Cas protein is catalytically inactive, for example due to both a D9A substitution and a N590A substitution, where the positions of the substitutions are defined with respect to the amino acid numbering of SEQ ID NO:14.

### 6.2.1.4. AXTQ Type II Cas Proteins

In one aspect, the disclosure provides AXTQ Type II Cas proteins. AXTQ Type II Cas proteins can be further classified as Type IIA Cas proteins. The AXTQ Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:19. In some embodiments, the AXTQ Type II Cas proteins comprise an amino acid sequence that is at least 95%, at least 96%, at least 97%, at least 98%, or at least 99% identical to SEQ ID NO:19. In some embodiments, an AXTQ Type II Cas protein comprises an amino acid sequence that is identical to SEQ ID NO:19.

Exemplary AXTQ Type II Cas protein sequences and nucleotide sequences encoding exemplary AXTQ proteins are set forth in Table 1D.

TABLE 1D
AXTQ Type II Cas Sequences

| | | SEQ ID |
|---|---|---|
| Name | Sequence | NO: |
| AXTQ Type II | IFGLDVGVASLGWAVIGEDYKVIETGVNSFKSADASMNVERRGFRQLRR | 19 |
| Cas amino | LHRRKKNRINDFNNLWVKTGMRIPSQKCNNQIELRVKGLKDKLTEEEIYF | |
| acid | VLVNMLKHRGISYLEDAVDESNKNESKYEKGINENQKLLQEGKFPCEIQL | |
| sequence | SRLEKHGQYRGEIVENDIILGNVFTTKAYRKEVVALLENQTKSHQFITDEF | |
| (without N- | KSAYLNIFNRKRKYYEGPGNELSRTDYGIYTTVIDESTGEYKTEDNIFDKLI | |
| terminal | GKCSVYKNLRRAAGASYTSQEFNLLNDLNNLTVNGRKLEEVEKHRIVEII | |
| methionine) | KTSNTVNMKNIIKKVINENINTLSGARIDKDGKEIFHKFEQYNKIRKELEKIG | |
| | GDITNFSREDLDEIGEILTLNTEKEAILSALRKIPCNDQEKECFFNLRRSNP | |
| | GMFNKWQSFSLELMNELIPEMYLQSKNQMQLLTDMGVFKLSSDRYNGY | |
| | KYIPKEVVTQEIYNPVVKRSIRITIDVMNALIKKYGYPEKIVIEMPRDKNSDD | |
| | EKNRIKEEQKKNEREITEVLEKIKNEYGIVITDEHRKRQNQLNLKLKLWNE | |
| | QDGRCLYSGKEIDINDLLYDPSKFEIDHIIPRSISFDDSRSNKVLVYYTENK | |
| | KKGNLTPYMYLIGQNRDWDWHKYMNKVLELKANAKISKSKVDKLLFTDEI | |
| | TKIDVLKGFVSRNINDTRYASKVILNALQSFMQSKNVDTKVATIKGSFTHQ | |
| | MRKVLRLEKNRNESFAHHAIDAMLICYSQMGFDTYRKLQETFIDFDNEDI | |
| | TDKNLWDRYMDDRVCEEYLYNKKWRMIKENIQNAEKNIKYWYKVDSKV | |
| | NRGLCNQTIVGTREINGEIYKINKLNLYTPDGYNRFKKIIDSGKEKDFLMY | |
| | SNDRRTFNDILKIYRQYNDSTNAFMQYYNETGDFVRKYAKNHNGCRIEN | |
| | LKYYEKKIESCIDISHKYGHDVGSKKVVLLGLNPYRMDVYYNTNSNKYYF | |
| | VGVKFSDLRFQGGTYLVDKDMYSQALINEKMLQPGEDISMLQSKGYEFR | |
| | LTFYKNEIIKYEKKGEMFVERFLSRTSQDSNGIETKPINAEKFKNKDRNIV | |
| | KLAKTKFVSKIRTDILGNQYECLKEEFKLMIDNVD | |
| AXTQ Type II | MIFGLDVGVASLGWAVIGEDYKVIETGVNSFKSADASMNVERRGFRQLR | 20 |
| Cas amino | RLHRRKKNRINDFNNLWVKTGMRIPSQKCNNQIELRVKGLKDKLTEEEIY | |
| acid | FVLVNMLKHRGISYLEDAVDESNKNESKYEKGINENQKLLQEGKFPCEIQ | |
| sequence | LSRLEKHGQYRGEIVENDIILGNVFTTKAYRKEVVALLENQTKSHQFITDE | |
| | FKSAYLNIFNRKRKYYEGPGNELSRTDYGIYTTVIDESTGEYKTEDNIFDK | |
| | LIGKCSVYKNLRRAAGASYTSQEFNLLNDLNNLTVNGRKLEEVEKHRIVEI | |
| | IKTSNTVNMKNIIKKVINENINTLSGARIDKDGKEIFHKFEQYNKIRKELEKI | |
| | GGDITNFSREDLDEIGEILTLNTEKEAILSALRKIPCNDQEKECFFNLRRSN | |
| | PGMFNKWQSFSLELMNELIPEMYLQSKNQMQLLTDMGVFKLSSDRYNG | |
| | YKYIPKEVVTQEIYNPVVKRSIRITIDVMNALIKKYGYPEKIVIEMPRDKNSD | |
| | DEKNRIKEEQKKNEREITEVLEKIKNEYGIVITDEHRKRQNQLNLKLKLWN | |
| | EQDGRCLYSGKEIDINDLLYDPSKFEIDHIIPRSISFDDSRSNKVLVYYTEN | |
| | KKKGNLTPYMYLIGQNRDWDWHKYMNKVLELKANAKISKSKVDKLLFTD | |
| | EITKIDVLKGFVSRNINDTRYASKVILNALQSFMQSKNVDTKVATIKGSFTH | |
| | QMRKVLRLEKNRNESFAHHAIDAMLICYSQMGFDTYRKLQETFIDFDNE | |
| | DITDKNLWDRYMDDRVCEEYLYNKKWRMIKENIQNAEKNIKYWYKVDSK | |
| | VNRGLCNQTIVGTREINGEIYKINKLNLYTPDGYNRFKKIIDSGKEKDFLM | |
| | YSNDRRTFNDILKIYRQYNDSTNAFMQYYNETGDFVRKYAKNHNGCRIE | |
| | NLKYYEKKIESCIDISHKYGHDVGSKKVVLLGLNPYRMDVYYNTNSNKYY | |
| | FVGVKFSDLRFQGGTYLVDKDMYSQALINEKMLQPGEDISMLQSKGYEF | |
| | RLTFYKNEIIKYEKKGEMFVERFLSRTSQDSNGIETKPINAEKFKNKDRNI | |
| | VKLAKTKFVSKIRTDILGNQYECLKEEFKLMIDNVD | |
| AXTQ Type II | MIPNPLLGLDKRTADGSEFESPKKKRKVGSIFGLDVGVASLGWAVIGEDY | 21 |
| Cas | KVIETGVNSFKSADASMNVERRGFRQLRRLHRRKKNRINDFNNLWVKTG | |
| mammalian | MRIPSQKCNNQIELRVKGLKDKLTEEEIYFVLVNMLKHRGISYLEDAVDES | |
| expression | NKNESKYEKGINENQKLLQEGKFPCEIQLSRLEKHGQYRGEIVENDIILGN | |
| construct | VFTTKAYRKEVVALLENQTKSHQFITDEFKSAYLNIFNRKRKYYEGPGNE | |
| (includes N- | LSRTDYGIYTTVIDESTGEYKTEDNIFDKLIGKCSVYKNLRRAAGASYTSQ | |
| terminal SV5 | EFNLLNDLNNLTVNGRKLEEVEKHRIVEIIKTSNTVNMKNIIKKVINENINTL | |
| tag and NLS | SGARIDKDGKEIFHKFEQYNKIRKELEKIGGDITNFSREDLDEIGEILTLNT | |
| and C- | EKEAILSALRKIPCNDQEKECFFNLRRSNPGMFNKWQSFSLELMNELIPE | |
| terminal NLS) | MYLQSKNQMQLLTDMGVFKLSSDRYNGYKYIPKEVVTQEIYNPVVKRSI | |
| (aa) | RITIDVMNALIKKYGYPEKIVIEMPRDKNSDDEKNRIKEEQKKNEREITEVL | |
| | EKIKNEYGIVITDEHRKRQNQLNLKLKLWNEQDGRCLYSGKEIDINDLLYD | |
| | PSKFEIDHIIPRSISFDDSRSNKVLVYYTENKKKGNLTPYMYLIGQNRDWD | |
| | WHKYMNKVLELKANAKISKSKVDKLLFTDEITKIDVLKGFVSRNINDTRYA | |
| | SKVILNALQSFMQSKNVDTKVATIKGSFTHQMRKVLRLEKNRNESFAHH | |
| | AIDAMLICYSQMGFDTYRKLQETFIDFDNEDITDKNLWDRYMDDRVCEEY | |
| | LYNKKWRMIKENIQNAEKNIKYWYKVDSKVNRGLCNQTIVGTREINGEIY | |
| | KINKLNLYTPDGYNRFKKIIDSGKEKDFLMYSNDRRTFNDILKIYRQYNDS | |
| | TNAFMQYYNETGDFVRKYAKNHNGCRIENLKYYEKKIESCIDISHKYGHD | |
| | VGSKKVVLLGLNPYRMDVYYNTNSNKYYFVGVKFSDLRFQGGTYLVDK | |
| | DMYSQALINEKMLQPGEDISMLQSKGYEFRLTFYKNEIIKYEKKGEMFVE | |
| | RFLSRTSQDSNGIETKPINAEKFKNKDRNIVKLAKTKFVSKIRTDILGNQYE | |
| | CLKEEFKLMIDNVDSRKRTADGSEFESPKKKRKV | |
| AXTQ Type II | ATGATATTTGGATTAGATGTTGGAGTTGCGTCTTTAGGATGGGCAGTT | 22 |
| Cas coding | ATAGGTGAAGATTATAAAGTAATAGAAACAGGTGTAAATTCTTTTAAGT | |
| sequence (nt) | CGGCAGATGCAAGCATGAATGTTGAAAGACGAGGATTTAGGCAATTA | |
| not codon | AGAAGATTGCATAGAAGAAAGAAGAATAGAATAAATGATTTTAATAATT | |
| optimized | TGTGGGTTAAGACGGGGATGAGAATACCTAGTCAAAAATGTAACAAC | |
| | CAAATCGAACTCAGAGTAAAAGGCTTAAAGGATAAATTAACCGAAGAA | |
| | GAGATTTATTTTGTCCTTGTAAATATGTTAAAGCATAGGGGAATATCAT | |
| | ATCTTGAAGATGCAGTAGATGAATCCAACAAGAACGAAAGCAAATATG | |
| | AAAAAGGAATAAATGAAAATCAAAAGTTATTGCAAGAGGGCAAATTCC | |
| | CTTGTGAAATACAGTTAAGTAGGCTTGAAAAGCATGGTCAATATAGAG | |
| | GGGAAATTGTAGAAAATGATATTATATTGGGGAACGTTTTTACAACAA | |
| | AAGCTTATAGAAAAGAGGTGGTAGCATTATTGGAAAATCAGACAAAGT | |
| | CACATCAGTTTATCACAGATGAGTTTAAGTCAGCTTATTTAAATATATT | |
| | TAATAGAAAAAGAAAATATTATGAAGGACCGGGAAATGAATTATCAAG | |
| | GACGGATTATGGAATATATACAACTGTGATAGACGAAAGTACAGGTGA | |
| | ATATAAAACGGAGGATAACATATTTGATAAGTTAATTGGAAAATGTTCG | |
| | GTTTATAAGAATTTAAGAAGAGCTGCGGGAGCATCATATACATCACAG | |
| | GAATTTAATCTTTTAAATGACTTAAATAACCTGACCGTTAATGGAAGAA | |
| | AGCTAGAGGAAGTGGAGAAACACAGAATAGTTGAAATTATTAAAACAT | |
| | CAAACACTGTAAATATGAAAAATATTATAAAAAAAGTGATAAATGAGAA | |
| | TATTAATACATTGTCAGGTGCTAGAATCGATAAAGATGGAAAAGAAAT | |
| | TTTTCACAAATTTGAGCAATATAACAAAATACGAAAAGAATTAGAAAAA | |
| | ATAGGAGGGGATATAACAAATTTTTCTAGAGAGGATTTGGATGAAATC | |
| | GGAGAAATTTTGACACTAAATACAGAAAAAGAGGCAATCTTATCGGCG | |
| | TTGAGAAAAATACCATGCAACGATCAGGAGAAAGAGTGTTTCTTTAAT | |
| | TTAAGACGCTCAAATCCTGGTATGTTTAATAAGTGGCAGTCTTTTTCG | |
| | CTGGAATTAATGAACGAACTTATTCCTGAAATGTATTTGCAATCAAAGA | |
| | ATCAGATGCAACTACTTACTGATATGGGAGTGTTTAAATTATCAAGTG | |
| | ACAGATATAATGGGTATAAATATATACCCAAAGAAGTAGTGACACAAG | |
| | AAATATATAATCCGGTGGTTAAACGTTCAATAAGGATAACAATAGATG | |
| | TTATGAATGCTCTTATAAAAAAATATGGTTATCCGGAAAAGATAGTTAT | |
| | AGAAATGCCTAGAGATAAAAATTCTGATGATGAAAAAAATAGGATAAA | |
| | AGAAGAACAAAAGAAAAATGAAAGGGAGATAACAGAGGTACTTGAAA | |
| | AAATAAAGAATGAATATGGTATTGTAATAACAGATGAACATCGCAAAC | |
| | GACAGAACCAGTTAAATTTGAAGCTTAAATTATGGAATGAGCAGGATG | |
| | GGAGGTGTCTTTATTCAGGAAAAGAAATAGATATTAATGATTTACTGTA | |
| | TGATCCATCTAAATTTGAAATCGATCATATTATTCCACGATCAATATCA | |
| | TTTGATGATAGTCGTAGCAATAAAGTTCTGGTGTACTATACAGAAAAT | |
| | AAAAAAAAGGGAAATCTAACGCCATATATGTATCTTATTGGTCAAAATA | |
| | GAGATTGGGACTGGCATAAATATATGAATAAAGTTTTGGAATTAAAAG | |
| | CAAATGCCAAAATTTCCAAATCTAAAGTTGATAAATTGTTATTTACAGA | |
| | TGAAATTACTAAGATAGATGTATTGAAGGGATTTGTATCACGTAATATT | |
| | AATGATACGAGGTATGCATCTAAGGTTATTCTTAACGCGTTGCAGTCA | |
| | TTTATGCAATCAAAAAATGTAGACACAAAGGTAGCAACTATTAAAGGA | |
| | AGCTTTACTCATCAAATGAGGAAAGTTCTGAGACTGGAAAAGAATAGA | |
| | AATGAATCCTTCGCACATCATGCTATAGATGCAATGTTAATTTGTTATT | |
| | CACAAATGGGATTTGATACATATCGAAAGTTACAAGAAACATTTATTGA | |
| | TTTTGATAATGAGGATATAACAGATAAAAATCTGTGGGATAGATATATG | |
| | GATGATAGGGTCTGCGAAGAATATTTGTATAATAAAAAATGGAGAATG | |
| | ATTAAGGAAAATATACAAAACGCTGAAAAAAATATTAAGTATTGGTACA | |
| | AAGTAGATTCTAAAGTCAATAGAGGATTATGCAATCAAACAATAGTGG | |
| | GAACACGGGAAATAAATGGAGAAATATATAAAATTAATAAACTTAATCT | |
| | ATATACGCCAGATGGATATAATAGATTCAAAAAAATAATAGATTCAGGT | |
| | AAAGAAAAAGACTTTTTGATGTATAGTAATGACAGAAGAACATTTAAC | |
| | GATATATTAAAAATATACCGTCAATATAATGATTCAACAAATGCTTTTAT | |
| | GCAGTACTACAATGAAACAGGGGATTTTGTCAGAAAATATGCTAAAAA | |
| | TCATAATGGGTGCAGAATCGAAAATCTTAAATATTATGAAAAAAAGATT | |
| | GAGTCATGTATAGATATTTCGCATAAGTATGGGCATGATGTTGGAAGT | |
| | AAAAAAGTTGTTCTTTTAGGGCTTAACCCATATAGAATGGATGTATATT | |
| | ATAACACTAATAGCAATAAGTATTATTTTGTAGGAGTTAAATTTTCAGA | |
| | TTTAAGATTTCAAGGTGGTACATATTTGGTTGATAAAGATATGTATTCG | |
| | CAAGCGCTTATAAATGAGAAAATGTTACAGCCTGGAGAAGACATAAGT | |
| | ATGTTGCAGAGCAAAGGCTACGAATTCAGGTTGACGTTTTACAAAAAT | |
| | GAGATAATTAAATATGAAAAAAAAGGAGAAATGTTCGTTGAAAGGTTT | |
| | CTTTCGAGGACTTCGCAAGATAGTAATGGTATAGAAACAAAACCTATA | |
| | AACGCCGAAAAATTTAAAAATAAAGATAGGAATATAGTTAAACTTGCAA | |
| | AAACAAAATTTGTCAGTAAAATCCGTACAGATATATTAGGAAATCAGTA | |
| | TGAATGTTTAAAAGAAGAATTTAAATTAATGATTGACAATGTGGACTAG | |
| AXTQ Type II | ATCTTCGGCCTGGATGTAGGAGTGGCCAGCCTGGGATGGGCCGTGA | 23 |
| Cas coding | TCGGCGAGGACTACAAGGTGATCGAGACTGGCGTTAATTCTTTTAAG | |
| sequence (nt) | TCTGCCGACGCCTCTATGAATGTGGAAAGAAGAGGATTTAGACAGCT | |
| codon | GAGAAGATTGCACAGAAGAAAGAAGAACAGAATCAACGACTTTAACA | |
| optimized | ATCTGTGGGTCAAGACAGGCATGAGAATCCCTAGCCAGAAGTGCAAC | |
| | AACCAGATCGAGCTGAGAGTGAAGGGCCTGAAGGACAAGCTCACCG | |
| | AGGAAGAGATCTACTTCGTGCTGGTTAACATGCTGAAACACAGGGGC | |
| | ATCTCTTACCTGGAGGACGCCGTGGATGAGTCCAACAAAAATGAGAG | |
| | CAAGTACGAGAAGGGCATCAACGAAAATCAGAAACTGCTGCAGGAGG | |
| | GCAAGTTCCCTTGTGAAATCCAGCTGAGCAGACTGGAGAAACACGGC | |
| | CAGTATAGGGGCGAGATCGTGGAGAACGACATCATCCTGGGCAACG | |
| | TGTTCACAACCAAAGCCTACAGAAAGGAGGTGGTGGCCCTGCTGGAA | |
| | AACCAGACCAAGTCTCACCAGTTCATCACCGATGAGTTCAAGAGCGC | |
| | CTATCTGAACATCTTTAATAGAAAGCGGAAGTACTACGAGGGACCTG | |
| | GCAATGAACTGAGCAGAACCGATTACGGCATCTACACCACAGTGATT | |
| | GACGAGTCCACCGGCGAATACAAGACCGAGGACAACATCTTCGACAA | |
| | GCTGATCGGCAAGTGCAGCGTGTACAAGAACCTGAGAAGAGCCGCC | |
| | GGCGCCAGCTACACCTCTCAAGAGTTCAACCTTCTCAACGACCTGAA | |
| | TAACCTGACAGTGAACGGCAGAAAGCTCGAAGAGGTGGAAAAACATA | |
| | GAATCGTCGAGATCATCAAGACTTCTAACACCGTGAACATGAAGAATA | |
| | TAATCAAGAAGGTGATCAACGAAAACATCAACACCCTCAGCGGCGCC | |
| | AGAATCGACAAGGACGGCAAGGAGATCTTCCACAAGTTCGAGCAATA | |
| | CAACAAGATCCGGAAAGAGCTGGAGAAGATCGGAGGCGATATCACC | |
| | AACTTCAGCAGAGAGGACCTGGACGAGATCGGCGAGATCCTGACCC | |
| | TGAACACCGAGAAAGAGGCCATCCTGTCTGCCCTGAGAAAGATCCCA | |
| | TGCAACGACCAGGAAAAAGAATGCTTCTTCAACCTGAGAAGATCTAAC | |
| | CCCGGCATGTTCAACAAGTGGCAGTCTTTCAGCCTGGAACTGATGAA | |
| | CGAACTGATCCCCGAGATGTACCTGCAGAGCAAGAACCAAATGCAGC | |
| | TGCTGACCGATATGGGGGTCTTTAAGCTGAGTTCTGATAGATACAATG | |
| | GCTACAAGTACATTCCCAAGGAAGTGGTGACCCAGGAGATCTATAAT | |
| | CCTGTGGTGAAACGGTCCATCAGGATTACAATCGACGTGATGAACGC | |
| | CCTGATCAAGAAATATGGATACCCTGAGAAGATCGTGATCGAGATGC | |
| | CACGGGACAAAAACAGCGACGACGAGAAGAACAGAATCAAAGAAGA | |
| | GCAGAAGAAGAACGAGCGCGAGATCACAGAAGTGCTCGAAAAGATC | |
| | AAGAACGAATACGGAATTGTGATCACCGATGAGCACAGAAAGAGACA | |
| | GAACCAGCTGAATCTCAAACTGAAGCTGTGGAACGAACAAGACGGAA | |
| | GATGCCTGTACTCCGGCAAAGAAATCGACATCAACGATCTCCTGTAC | |
| | GACCCCTCCAAGTTTGAGATCGATCACATCATTCCTAGATCCATCAGC | |
| | TTCGACGATAGCCGGAGCAACAAGGTGCTGGTGTATTACACCGAGAA | |
| | CAAGAAGAAGGGCAACCTGACCCCCTACATGTACCTGATCGGACAGA | |
| | ACCGGGATTGGGACTGGCACAAGTATATGAACAAAGTGCTGGAACTG | |
| | AAGGCCAATGCTAAGATCAGCAAGAGCAAGGTGGACAAGCTGCTGTT | |
| | CACAGACGAGATAACCAAGATCGACGTGCTGAAGGGTTTCGTGAGCC | |
| | GGAACATCAACGACACCAGATACGCCAGCAAGGTGATCCTGAACGCA | |
| | CTGCAGTCTTTCATGCAAAGCAAGAACGTGGATACAAAGGTGGCTAC | |
| | AATCAAGGGCTCTTTTACCCACCAGATGCGGAAGGTGCTGAGACTGG | |
| | AAAAAAACAGAAATGAGTCCTTCGCCCACCACGCCATAGATGCTATGT | |
| | TAATTTGCTACAGCCAGATGGGCTTCGACACATACCGCAAGCTGCAG | |
| | GAGACATTTATCGACTTCGATAACGAGGATATCACAGACAAGAACCTG | |
| | TGGGATCGGTACATGGATGACAGAGTGTGCGAGGAGTACCTTTACAA | |
| | CAAGAAATGGCGGATGATCAAGGAAAACATTCAGAATGCCGAAAAGA | |
| | ACATCAAATACTGGTACAAGGTGGACTCCAAGGTGAACCGCGGACTT | |
| | TGTAACCAGACCATCGTGGGCACCCGGGAGATCAACGGCGAGATCT | |
| | ACAAGATAAATAAACTGAACCTGTACACACCTGATGGCTACAATAGAT | |
| | TCAAGAAAATTATCGACAGCGGCAAGGAAAAAGACTTTCTGATGTACA | |
| | GCAATGACAGACGGACCTTCAACGACATCCTGAAGATCTACCGGCAG | |
| | TACAACGACAGCACCAACGCCTTCATGCAGTACTACAACGAAACCGG | |
| | CGACTTCGTGCGGAAATACGCCAAAAACCACAACGGCTGTCGGATCG | |
| | AGAATCTGAAGTATTACGAAAAGAAGATCGAGAGCTGCATCGATATCA | |
| | GCCACAAGTACGGCCATGATGTTGGCAGCAAGAAAGTGGTGCTGCT | |
| | GGGCCTGAACCCTTACAGAATGGACGTGTACTATAATACCAATAGCA | |
| | ACAAGTACTATTTCGTGGGAGTGAAGTTCAGCGATCTGCGGTTTCAG | |
| | GGCGGCACCTACCTGGTGGACAAGGACATGTACAGCCAGGCCCTGA | |
| | TCAACGAAAAGATGCTGCAGCCTGGCGAGGATATTAGCATGCTGCAA | |
| | AGCAAGGGATACGAGTTCAGACTGACATTCTACAAGAATGAAATCATC | |
| | AAGTACGAGAAGAAGGGCGAAATGTTCGTGGAGAGATTCCTGAGCAG | |
| | AACCTCACAGGATTCCAACGGCATCGAGACAAAGCCTATCAACGCTG | |
| | AGAAATTCAAAAACAAAGACAGAAACATCGTGAAGCTGGCCAAGACC | |
| | AAGTTCGTGTCAAAGATCAGAACCGACATCCTGGGCAATCAGTACGA | |
| | GTGTTTAAAAGAGGAGTTCAAGCTGATGATCGACAACGTGGAC | |
| AXTQ Type II | ATGATCCCCAATCCCCTGCTGGGCCTGGACAAGCGGACTGCCGACG | 24 |
| Cas coding | GCAGTGAGTTCGAGTCTCCCAAGAAAAAGAGGAAAGTGggatccATCTT | |
| sequence (nt) | CGGCCTGGATGTAGGAGTGGCCAGCCTGGGATGGGCCGTGATCGG | |
| codon | CGAGGACTACAAGGTGATCGAGACTGGCGTTAATTCTTTTAAGTCTG | |
| optimized | CCGACGCCTCTATGAATGTGGAAAGAAGAGGATTTAGACAGCTGAGA | |
| (including | AGATTGCACAGAAGAAAGAAGAACAGAATCAACGACTTTAACAATCTG | |
| V5-tag and N- | TGGGTCAAGACAGGCATGAGAATCCCTAGCCAGAAGTGCAACAACCA | |
| and C- | GATCGAGCTGAGAGTGAAGGGCCTGAAGGACAAGCTCACCGAGGAA | |
| terminal NLS) | GAGATCTACTTCGTGCTGGTTAACATGCTGAAACACAGGGGCATCTC | |
| | TTACCTGGAGGACGCCGTGGATGAGTCCAACAAAAATGAGAGCAAGT | |
| | ACGAGAAGGGCATCAACGAAAATCAGAAACTGCTGCAGGAGGGCAA | |
| | GTTCCCTTGTGAAATCCAGCTGAGCAGACTGGAGAAACACGGCCAGT | |
| | ATAGGGGCGAGATCGTGGAGAACGACATCATCCTGGGCAACGTGTT | |
| | CACAACCAAAGCCTACAGAAAGGAGGTGGTGGCCCTGCTGGAAAAC | |
| | CAGACCAAGTCTCACCAGTTCATCACCGATGAGTTCAAGAGCGCCTA | |
| | TCTGAACATCTTTAATAGAAAGCGGAAGTACTACGAGGGACCTGGCA | |
| | ATGAACTGAGCAGAACCGATTACGGCATCTACACCACAGTGATTGAC | |
| | GAGTCCACCGGCGAATACAAGACCGAGGACAACATCTTCGACAAGCT | |
| | GATCGGCAAGTGCAGCGTGTACAAGAACCTGAGAAGAGCCGCCGGC | |
| | GCCAGCTACACCTCTCAAGAGTTCAACCTTCTCAACGACCTGAATAAC | |
| | CTGACAGTGAACGGCAGAAAGCTCGAAGAGGTGGAAAAACATAGAAT | |
| | CGTCGAGATCATCAAGACTTCTAACACCGTGAACATGAAGAATATAAT | |
| | CAAGAAGGTGATCAACGAAAACATCAACACCCTCAGCGGCGCCAGAA | |
| | TCGACAAGGACGGCAAGGAGATCTTCCACAAGTTCGAGCAATACAAC | |
| | AAGATCCGGAAAGAGCTGGAGAAGATCGGAGGCGATATCACCAACTT | |
| | CAGCAGAGAGGACCTGGACGAGATCGGCGAGATCCTGACCCTGAAC | |
| | ACCGAGAAAGAGGCCATCCTGTCTGCCCTGAGAAAGATCCCATGCAA | |
| | CGACCAGGAAAAAGAATGCTTCTTCAACCTGAGAAGATCTAACCCCG | |
| | GCATGTTCAACAAGTGGCAGTCTTTCAGCCTGGAACTGATGAACGAA | |
| | CTGATCCCCGAGATGTACCTGCAGAGCAAGAACCAAATGCAGCTGCT | |
| | GACCGATATGGGGGTCTTTAAGCTGAGTTCTGATAGATACAATGGCT | |
| | ACAAGTACATTCCCAAGGAAGTGGTGACCCAGGAGATCTATAATCCT | |
| | GTGGTGAAACGGTCCATCAGGATTACAATCGACGTGATGAACGCCCT | |
| | GATCAAGAAATATGGATACCCTGAGAAGATCGTGATCGAGATGCCAC | |
| | GGGACAAAAACAGCGACGACGAGAAGAACAGAATCAAAGAAGAGCA | |
| | GAAGAAGAACGAGCGCGAGATCACAGAAGTGCTCGAAAAGATCAAGA | |
| | ACGAATACGGAATTGTGATCACCGATGAGCACAGAAAGAGACAGAAC | |
| | CAGCTGAATCTCAAACTGAAGCTGTGGAACGAACAAGACGGAAGATG | |
| | CCTGTACTCCGGCAAAGAAATCGACATCAACGATCTCCTGTACGACC | |
| | CCTCCAAGTTTGAGATCGATCACATCATTCCTAGATCCATCAGCTTCG | |
| | ACGATAGCCGGAGCAACAAGGTGCTGGTGTATTACACCGAGAACAAG | |
| | AAGAAGGGCAACCTGACCCCCTACATGTACCTGATCGGACAGAACCG | |
| | GGATTGGGACTGGCACAAGTATATGAACAAAGTGCTGGAACTGAAGG | |
| | CCAATGCTAAGATCAGCAAGAGCAAGGTGGACAAGCTGCTGTTCACA | |
| | GACGAGATAACCAAGATCGACGTGCTGAAGGGTTTCGTGAGCCGGA | |
| | ACATCAACGACACCAGATACGCCAGCAAGGTGATCCTGAACGCACTG | |
| | CAGTCTTTCATGCAAAGCAAGAACGTGGATACAAAGGTGGCTACAAT | |
| | CAAGGGCTCTTTTACCCACCAGATGCGGAAGGTGCTGAGACTGGAAA | |
| | AAAACAGAAATGAGTCCTTCGCCCACCACGCCATAGATGCTATGTTAA | |
| | TTTGCTACAGCCAGATGGGCTTCGACACATACCGCAAGCTGCAGGAG | |
| | ACATTTATCGACTTCGATAACGAGGATATCACAGACAAGAACCTGTGG | |
| | GATCGGTACATGGATGACAGAGTGTGCGAGGAGTACCTTTACAACAA | |
| | GAAATGGCGGATGATCAAGGAAAACATTCAGAATGCCGAAAAGAACA | |
| | TCAAATACTGGTACAAGGTGGACTCCAAGGTGAACCGCGGACTTTGT | |
| | AACCAGACCATCGTGGGCACCCGGGAGATCAACGGCGAGATCTACA | |
| | AGATAAATAAACTGAACCTGTACACACCTGATGGCTACAATAGATTCA | |
| | AGAAAATTATCGACAGCGGCAAGGAAAAAGACTTTCTGATGTACAGC | |
| | AATGACAGACGGACCTTCAACGACATCCTGAAGATCTACCGGCAGTA | |
| | CAACGACAGCACCAACGCCTTCATGCAGTACTACAACGAAACCGGCG | |
| | ACTTCGTGCGGAAATACGCCAAAAACCACAACGGCTGTCGGATCGAG | |
| | AATCTGAAGTATTACGAAAAGAAGATCGAGAGCTGCATCGATATCAGC | |
| | CACAAGTACGGCCATGATGTTGGCAGCAAGAAAGTGGTGCTGCTGG | |
| | GCCTGAACCCTTACAGAATGGACGTGTACTATAATACCAATAGCAACA | |
| | AGTACTATTTCGTGGGAGTGAAGTTCAGCGATCTGCGGTTTCAGGGC | |
| | GGCACCTACCTGGTGGACAAGGACATGTACAGCCAGGCCCTGATCA | |
| | ACGAAAAGATGCTGCAGCCTGGCGAGGATATTAGCATGCTGCAAAGC | |
| | AAGGGATACGAGTTCAGACTGACATTCTACAAGAATGAAATCATCAAG | |
| | TACGAGAAGAAGGGCGAAATGTTCGTGGAGAGATTCCTGAGCAGAAC | |
| | CTCACAGGATTCCAACGGCATCGAGACAAAGCCTATCAACGCTGAGA | |
| | AATTCAAAAACAAAGACAGAAACATCGTGAAGCTGGCCAAGACCAAG | |
| | TTCGTGTCAAAGATCAGAACCGACATCCTGGGCAATCAGTACGAGTG | |
| | TTTAAAAGAGGAGTTCAAGCTGATGATCGACAACGTGGACtctagaAAG | |
| | CGGACAGCAGACGGCTCCGAATTTGAAAGCCCTAAGAAAAAGAGAAA | |
| | GGTGTGA | |

In some embodiments an AXTQ Type II Cas protein comprises an amino acid sequence of SEQ ID NO:19, SEQ ID NO:20, or SEQ ID NO:21. In some embodiments, an AXTQ Type II Cas protein has nickase activity, for example resulting from one or more amino acid substitutions relative to the sequence of SEQ ID NO:19, SEQ ID NO:20, or SEQ ID NO:21. In some embodiments, the one or more amino acid substitutions providing nickase activity is a D6A substitution, wherein the position of the D6A substitution is defined with respect to the amino acid numbering of SEQ ID NO:20. In some embodiments, the one or more amino acid substitutions providing nickase activity is a N611A substitution, wherein the position of the N611A substitution is defined with respect to the amino acid numbering of SEQ ID NO:20. In some embodiments, an AXTQ Type II Cas protein is catalytically inactive, for example due to both a D6A substitution and a N611A substitution, where the positions of the substitutions are defined with respect to the amino acid numbering of SEQ ID NO:20.

### 6.2.1.5. AIWM Type II Cas Proteins

In one aspect, the disclosure provides AIWM Type II Cas proteins. AIWM Type II Cas proteins can be further classified as Type IIA Cas proteins. The AIWM Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:25. In some embodiments, the AIWM Type II Cas proteins comprise an amino acid sequence that is at least 95%, at least 96%, at least 97%, at least 98%, or at least 99% identical to SEQ ID NO:25. In some embodiments, a AIWM Type II Cas protein comprises an amino acid sequence that is identical to SEQ ID NO:25.

Exemplary AIWM Type II Cas protein sequences and nucleotide sequences encoding exemplary AIWM Type II Cas proteins are set forth in Table 1E.

TABLE 1E
AIWM Type II Cas Sequences

| | | SEQ ID |
|---|---|---|
| Name | Sequence | NO |
| AIWM Type II | AKYILGLDMGITSVGWGLIDYDSGKIVDKGVRLFKEATAEDNLKRRTHRG | 25 |
| Cas amino | SRRLKRRRQQRIIELKRLLKKENILTDDYTPLNNVYYLRCKGLNEKLTNAE | |
| acid | LVSVLLHIAKIRGSSLEVVEDDEKKANEDKSSKANLSVNETKLRTSKEEIC | |
| sequence | EKQYKDLIENGKVRGINNIYKSSSYESELKKILANQGLSKEVNDKIINIIFRR | |
| (without N- | RDFNEGPGSMESPTIYGRFIPHENGEIEIIPSMIEKMQGKCSIYQNEPRAA | |
| terminal | KMSFTADLFNFLNDLNNLEIQRADNYDNYITPDEKKLIINEYIIKKGKITIQKL | |
| valine) | CDVLQVKKENITGFRVNKGEPILTEFKGYQKVLKALNRIIDTEIVDAVIDVL | |
| | TKYKDVEKRKEEIKKINNAEFSEEDIVKLSEITGIQQYHSLSYKAMKVIIPEL | |
| | YENPENQMEVITRLNLVKTSIDDLKGRTTIPSYSDRIYSPVAKRVHNETIK | |
| | WVNAVIKKYGDLDSIVIEMARDRNTAEERKNIEQAQKRNQENREKAKEIL | |
| | DVNYSFSKLDSKLVQKIMLYNQQDGKSMYSGKPINLNVLISDPNAYEIDHI | |
| | IPLSISLDDSIANKVLVYRSENQQKLNNTPLQYLRSGNSNGWSVDEFREV | |
| | VIKMYNDKKISLKKLQNLLCEKDITKQDVRKEFIERNLVDTRYASRVVLGL | |
| | LKDYFKANNKNTKVFTIRGQITSQFRKSRNLVKSRDCFYHHIVDALIIAYFR | |
| | KFNYINELMQYNFSYKVKIDEETGELIQYNDTLFSTKDDDFTIQLRKLNDL | |
| | NKYSEDIKISHKVDRKPNRQFSDETIYSARKINNDYMKISKYKDIYGEEGK | |
| | KFAKVIKDNLQKEKDLDKFLMKKVDPETFNLLVSIVINTTCGEKENPFVKY | |
| | CEENNIDFIRKKSKNGNGPIIRSIRYYDGKVNSHLDISQKYVNLSDDKKVV | |
| | LLQISPYRTDFYLTKEGNYKFLTIKYANIKENHNGYYIPKDVYESLKKSKKI | |
| | NDSDEFLFSLYRNDYLRITTNDPTENAPMIYRFVGTNNDLENKVEVKPIY | |
| | CDKNEENRIMKTIGKKVTKIEKFDVDVLGNKIAKKRDKCLQLEL | |
| AIWM Type II | VAKYILGLDMGITSVGWGLIDYDSGKIVDKGVRLFKEATAEDNLKRRTHR | 26 |
| Cas amino | GSRRLKRRRQQRIIELKRLLKKENILTDDYTPLNNVYYLRCKGLNEKLTNA | |
| acid | ELVSVLLHIAKIRGSSLEVVEDDEKKANEDKSSKANLSVNETKLRTSKEEI | |
| sequence | CEKQYKDLIENGKVRGINNIYKSSSYESELKKILANQGLSKEVNDKIINIIFR | |
| | RRDFNEGPGSMESPTIYGRFIPHENGEIEIIPSMIEKMQGKCSIYQNEPRA | |
| | AKMSFTADLFNFLNDLNNLEIQRADNYDNYITPDEKKLIINEYIIKKGKITIQ | |
| | KLCDVLQVKKENITGFRVNKGEPILTEFKGYQKVLKALNRIIDTEIVDAVID | |
| | VLTKYKDVEKRKEEIKKINNAEFSEEDIVKLSEITGIQQYHSLSYKAMKVIIP | |
| | ELYENPENQMEVITRLNLVKTSIDDLKGRTTIPSYSDRIYSPVAKRVHNETI | |
| | KVVNAVIKKYGDLDSIVIEMARDRNTAEERKNIEQAQKRNQENREKAKEIL | |
| | DVNYSFSKLDSKLVQKIMLYNQQDGKSMYSGKPINLNVLISDPNAYEIDHI | |
| | IPLSISLDDSIANKVLVYRSENQQKLNNTPLQYLRSGNSNGWSVDEFREV | |
| | VIKMYNDKKISLKKLQNLLCEKDITKQDVRKEFIERNLVDTRYASRVVLGL | |
| | LKDYFKANNKNTKVFTIRGQITSQFRKSRNLVKSRDCFYHHIVDALIIAYFR | |
| | KFNYINELMQYNFSYKVKIDEETGELIQYNDTLFSTKDDDFTIQLRKLNDL | |
| | NKYSEDIKISHKVDRKPNRQFSDETIYSARKINNDYMKISKYKDIYGEEGK | |
| | KFAKVIKDNLQKEKDLDKFLMKKVDPETFNLLVSIVINTTCGEKENPFVKY | |
| | CEENNIDFIRKKSKNGNGPIIRSIRYYDGKVNSHLDISQKYVNLSDDKKVV | |
| | LLQISPYRTDFYLTKEGNYKFLTIKYANIKENHNGYYIPKDVYESLKKSKKI | |
| | NDSDEFLFSLYRNDYLRITTNDPTENAPMIYRFVGTNNDLENKVEVKPIY | |
| | CDKNEENRIMKTIGKKVTKIEKFDVDVLGNKIAKKRDKCLQLEL | |
| AIWM Type II | MIPNPLLGLDKRTADGSEFESPKKKRKVGSAKYILGLDMGITSVGWGLID | 27 |
| Cas | YDSGKIVDKGVRLFKEATAEDNLKRRTHRGSRRLKRRRQQRIIELKRLLK | |
| mammalian | KENILTDDYTPLNNVYYLRCKGLNEKLTNAELVSVLLHIAKIRGSSLEVVE | |
| expression | DDEKKANEDKSSKANLSVNETKLRTSKEEICEKQYKDLIENGKVRGINNIY | |
| construct | KSSSYESELKKILANQGLSKEVNDKIINIIFRRRDFNEGPGSMESPTIYGRF | |
| (includes N- | IPHENGEIEIIPSMIEKMQGKCSIYQNEPRAAKMSFTADLFNFLNDLNNLEI | |
| terminal SV5 | QRADNYDNYITPDEKKLIINEYIIKKGKITIQKLCDVLQVKKENITGFRVNKG | |
| tag and NLS | EPILTEFKGYQKVLKALNRIIDTEIVDAVIDVLTKYKDVEKRKEEIKKINNAE | |
| and C- | FSEEDIVKLSEITGIQQYHSLSYKAMKVIIPELYENPENQMEVITRLNLVKT | |
| terminal NLS) | SIDDLKGRTTIPSYSDRIYSPVAKRVHNETIKVVNAVIKKYGDLDSIVIEMA | |
| (aa) | RDRNTAEERKNIEQAQKRNQENREKAKEILDVNYSFSKLDSKLVQKIMLY | |
| | NQQDGKSMYSGKPINLNVLISDPNAYEIDHIIPLSISLDDSIANKVLVYRSE | |
| | NQQKLNNTPLQYLRSGNSNGWSVDEFREVVIKMYNDKKISLKKLQNLLC | |
| | EKDITKQDVRKEFIERNLVDTRYASRVVLGLLKDYFKANNKNTKVFTIRG | |
| | QITSQFRKSRNLVKSRDCFYHHIVDALIIAYFRKFNYINELMQYNFSYKVKI | |
| | DEETGELIQYNDTLFSTKDDDFTIQLRKLNDLNKYSEDIKISHKVDRKPNR | |
| | QFSDETIYSARKINNDYMKISKYKDIYGEEGKKFAKVIKDNLQKEKDLDKF | |
| | LMKKVDPETFNLLVSIVINTTCGEKENPFVKYCEENNIDFIRKKSKNGNGP | |
| | IIRSIRYYDGKVNSHLDISQKYVNLSDDKKVVLLQISPYRTDFYLTKEGNY | |
| | KFLTIKYANIKENHNGYYIPKDVYESLKKSKKINDSDEFLFSLYRNDYLRIT | |
| | TNDPTENAPMIYRFVGTNNDLENKVEVKPIYCDKNEENRIMKTIGKKVTKI | |
| | EKFDVDVLGNKIAKKRDKCLQLELSRKRTADGSEFESPKKKRKV | |
| AIWM Type II | GTGGCAAAATATATATTAGGACTAGATATGGGGATTACTTCGGTAGGT | 28 |
| Cas coding | TGGGGCCTAATTGACTATGATTCTGGAAAAATAGTTGATAAAGGGGTT | |
| sequence (nt) | AGACTTTTTAAAGAAGCAACTGCGGAAGACAATTTAAAAAGACGTACA | |
| (not codon | CATCGTGGATCAAGAAGGTTAAAAAGACGTAGACAACAAAGAATAATT | |
| optimized) | GAATTAAAACGATTATTGAAAAAAGAAAACATCCTTACAGATGATTATA | |
| | CTCCTTTAAATAATGTTTACTATCTTAGATGTAAAGGTTTAAATGAAAA | |
| | ATTAACAAATGCCGAACTTGTATCAGTATTATTACATATAGCAAAAATA | |
| | AGAGGAAGTTCATTAGAAGTAGTTGAAGATGATGAAAAAAAGGCAAAT | |
| | GAAGATAAAAGTAGTAAAGCCAATTTATCAGTAAATGAAACAAAACTA | |
| | AGGACTTCAAAAGAAGAAATTTGTGAAAAACAGTATAAAGATCTAATC | |
| | GAAAATGGTAAAGTTCGTGGAATAAATAATATTTATAAAAGTTCTTCAT | |
| | ATGAGTCAGAACTCAAAAAAATTTTAGCTAATCAAGGCTTGTCAAAAG | |
| | AAGTTAATGATAAGATAATAAATATAATTTTTAGAAGACGTGACTTTAA | |
| | TGAAGGACCAGGAAGCATGGAATCGCCTACTATATATGGTAGATTTAT | |
| | ACCACATGAAAATGGTGAGATAGAAATTATACCTAGTATGATTGAAAA | |
| | AATGCAAGGGAAATGTTCTATATACCAAAATGAACCTCGTGCAGCAAA | |
| | AATGTCATTTACAGCTGATTTGTTTAATTTCTTAAACGATTTAAACAATT | |
| | TAGAAATACAGCGTGCTGATAACTATGATAATTATATTACCCCAGACG | |
| | AAAAAAAACTAATAATAAATGAGTATATCATAAAAAAAGGTAAAATTAC | |
| | AATTCAAAAATTATGCGATGTACTACAGGTAAAAAAAGAAAATATTACT | |
| | GGCTTTAGAGTTAACAAAGGTGAACCAATACTCACTGAATTTAAAGGT | |
| | TATCAAAAAGTATTAAAAGCTTTAAATAGAATAATTGATACTGAAATTG | |
| | TTGATGCGGTTATTGATGTTTTAACAAAATATAAAGATGTAGAAAAAAG | |
| | AAAAGAAGAAATTAAAAAAATAAACAATGCAGAGTTTAGTGAGGAAGA | |
| | CATTGTAAAATTATCTGAAATAACAGGTATACAACAGTATCATTCATTG | |
| | TCTTATAAAGCAATGAAAGTAATAATACCTGAATTATATGAAAATCCTG | |
| | AAAATCAAATGGAAGTAATTACAAGATTAAATCTAGTGAAAACTTCAAT | |
| | AGATGATTTGAAAGGAAGAACAACAATACCTTCATATAGCGACAGAAT | |
| | ATATTCCCCTGTAGCTAAACGTGTACATAATGAGACAATCAAAGTAGT | |
| | AAATGCGGTAATAAAAAAATATGGAGATTTAGATAGTATTGTTATAGAA | |
| | ATGGCAAGGGATAGAAATACTGCTGAAGAAAGGAAAAATATTGAACAA | |
| | GCCCAAAAACGAAATCAAGAAAATAGAGAAAAAGCAAAAGAGATACTA | |
| | GATGTTAATTATAGTTTTTCGAAACTTGATTCAAAATTAGTTCAAAAGA | |
| | TCATGCTTTATAATCAACAAGATGGTAAAAGTATGTATAGTGGAAAAC | |
| | CAATAAACTTAAATGTTTTAATAAGTGACCCTAATGCATACGAGATAGA | |
| | TCATATCATACCATTATCAATTTCGCTTGATGATTCAATTGCCAATAAA | |
| | GTTCTGGTGTATAGAAGTGAAAACCAACAAAAATTAAATAATACACCA | |
| | CTTCAATACTTACGTAGTGGTAATTCAAATGGTTGGAGCGTCGACGAA | |
| | TTCCGTGAAGTTGTAATAAAAATGTATAATGATAAAAAAATATCACTAA | |
| | AAAAATTACAAAACTTACTATGTGAAAAAGATATAACAAAACAAGATGT | |
| | AAGAAAAGAATTTATTGAAAGAAATTTAGTTGACACAAGATATGCAAGT | |
| | AGGGTAGTTTTGGGACTTCTAAAAGATTATTTCAAAGCCAACAATAAA | |
| | AATACTAAAGTATTTACAATAAGAGGCCAAATAACATCACAGTTCAGA | |
| | AAAAGTAGAAACTTGGTGAAAAGTAGAGATTGCTTTTATCATCATATA | |
| | GTAGATGCATTAATAATTGCATATTTTAGAAAATTCAATTACATTAATG | |
| | AATTAATGCAATACAATTTTAGTTATAAAGTGAAAATTGATGAAGAAAC | |
| | AGGAGAATTAATCCAATATAATGATACGTTGTTTTCAACGAAGGATGA | |
| | TGATTTCACAATACAATTAAGAAAACTAAATGACCTTAATAAATATAGT | |
| | GAAGATATCAAAATTTCGCATAAAGTTGATAGAAAGCCTAATAGACAG | |
| | TTTAGTGATGAAACAATCTATTCTGCGAGAAAAATCAATAACGATTATA | |
| | TGAAAATTTCGAAATATAAAGATATTTATGGTGAAGAAGGTAAAAAATT | |
| | TGCAAAAGTAATTAAAGATAATCTCCAAAAAGAAAAAGACTTAGATAAA | |
| | TTCTTAATGAAGAAGGTAGATCCAGAAACTTTTAATTTATTAGTTAGCA | |
| | TTGTTATAAATACAACATGTGGTGAAAAAGAAAACCCTTTTGTTAAATA | |
| | TTGTGAAGAAAACAATATAGATTTTATTAGAAAAAAATCTAAAAATGGT | |
| | AATGGTCCGATTATTAGGTCTATTAGATATTATGATGGTAAAGTAAATA | |
| | GTCACTTGGATATCTCACAAAAATATGTAAACTTATCTGATGACAAAAA | |
| | AGTGGTGCTTTTACAAATTTCACCATATAGAACTGATTTTTACTTAACT | |
| | AAAGAAGGAAACTATAAATTTCTAACAATTAAATATGCTAATATCAAAG | |
| | AAAACCATAATGGATATTATATACCCAAAGATGTTTACGAATCATTAAA | |
| | GAAATCAAAGAAAATAAATGATAGTGATGAATTTTTATTTTCTCTATAT | |
| | CGAAATGATTACCTAAGAATTACAACAAATGATCCTACTGAAAATGCT | |
| | CCTATGATTTATCGGTTTGTAGGTACAAATAATGATTTAGAAAACAAAG | |
| | TTGAAGTAAAACCAATTTATTGTGATAAAAATGAAGAAAATCGTATTAT | |
| | GAAAACCATAGGTAAAAAAGTGACTAAAATTGAAAAGTTTGATGTTGA | |
| | TGTACTAGGTAATAAAATTGCTAAAAAAAGAGATAAATGCTTGCAATTA | |
| | GAGCTGTAG | |
| AIWM Type II | GCTAAGTACATCCTGGGACTGGACATGGGCATCACAAGCGTGGGCT | 29 |
| Cas coding | GGGGCCTGATCGATTACGACAGCGGCAAGATCGTGGACAAGGGCGT | |
| sequence (nt) | GCGGCTGTTTAAGGAGGCCACAGCCGAGGACAACCTGAAGAGAAGG | |
| (human | ACCCACAGAGGCTCCCGGAGACTGAAGCGGCGGAGACAGCAAAGAA | |
| codon- | TTATAGAGCTGAAGCGCCTGCTGAAAAAAGAAAATATCCTGACAGATG | |
| optimized) | ACTACACCCCTCTGAACAATGTGTACTATCTGAGGTGTAAAGGACTCA | |
| | ACGAGAAACTGACAAACGCCGAGCTGGTGTCCGTCCTGCTTCATATC | |
| | GCCAAGATTCGGGGCAGCAGCCTGGAAGTGGTCGAAGATGACGAAA | |
| | AAAAGGCCAACGAAGATAAGAGCAGCAAGGCCAACCTGTCCGTGAAT | |
| | GAAACCAAGCTGAGAACAAGCAAAGAAGAGATCTGCGAGAAGCAATA | |
| | CAAAGACCTGATCGAGAACGGCAAGGTCAGAGGAATCAACAACATCT | |
| | ATAAGAGCTCTAGCTATGAAAGCGAGCTGAAGAAAATTCTGGCCAAT | |
| | CAGGGCCTGAGCAAGGAGGTGAACGATAAGATCATCAACATCATTTT | |
| | CAGACGGCGGGACTTCAACGAAGGCCCCGGCAGCATGGAGAGCCCA | |
| | ACAATCTACGGCAGGTTCATCCCACACGAGAACGGCGAAATCGAAAT | |
| | CATTCCTTCTATGATCGAAAAGATGCAGGGCAAGTGCAGCATCTACC | |
| | AGAACGAACCTAGAGCCGCGAAGATGAGCTTCACCGCCGATCTGTTC | |
| | AATTTCCTGAACGACCTGAACAATTTGGAGATCCAGAGAGCCGACAA | |
| | CTACGACAACTACATCACCCCTGACGAGAAGAAACTGATCATCAACG | |
| | AGTACATCATTAAGAAGGGCAAGATCACCATCCAGAAGCTGTGCGAC | |
| | GTGCTGCAAGTGAAGAAGGAGAACATCACCGGATTCAGAGTGAACAA | |
| | GGGCGAACCCATCCTGACCGAGTTCAAGGGATATCAGAAGGTGCTG | |
| | AAAGCTCTGAACAGAATCATTGATACCGAGATCGTGGATGCCGTGAT | |
| | CGATGTGCTGACCAAATACAAGGACGTGGAAAAACGGAAAGAGGAAA | |
| | TCAAAAAGATCAACAATGCTGAGTTCAGCGAGGAAGATATCGTGAAG | |
| | CTGAGCGAGATCACAGGCATCCAGCAGTACCACAGCCTGTCCTACAA | |
| | GGCCATGAAGGTGATCATCCCTGAGCTGTATGAGAACCCCGAGAACC | |
| | AGATGGAAGTGATCACCAGACTCAATCTGGTGAAAACCAGCATCGAC | |
| | GACCTGAAGGGCAGAACCACCATCCCCAGCTACTCCGACCGGATCTA | |
| | CAGCCCTGTGGCCAAAAGAGTGCACAACGAGACAATCAAGGTGGTG | |
| | AACGCAGTGATCAAGAAGTACGGCGACCTTGACAGCATCGTGATCGA | |
| | GATGGCCAGAGACAGAAACACCGCCGAAGAAAGAAAGAACATCGAA | |
| | CAGGCCCAGAAGCGGAACCAGGAGAATAGAGAGAAAGCTAAAGAAA | |
| | TCCTGGACGTAAACTACTCTTTTTCAAAGCTGGATTCTAAGCTGGTCC | |
| | AGAAAATCATGCTGTACAATCAACAAGACGGAAAGTCCATGTACAGC | |
| | GGCAAGCCCATCAATCTGAACGTCCTGATCAGCGACCCTAACGCCTA | |
| | CGAGATCGACCACATCATCCCCCTGTCTATCAGCCTGGACGACAGCA | |
| | TCGCCAACAAAGTGCTGGTTTACAGAAGCGAGAACCAGCAGAAGCTG | |
| | AACAACACACCCCTGCAGTACCTGAGAAGCGGCAATAGCAACGGCTG | |
| | GAGCGTGGACGAGTTCAGAGAAGTGGTGATCAAGATGTACAACGACA | |
| | AAAAAATTTCCCTCAAAAAACTGCAGAACCTGCTGTGCGAGAAGGAC | |
| | ATCACCAAGCAGGACGTGCGAAAGGAATTTATCGAGCGGAACCTGGT | |
| | CGATACAAGATACGCCAGCCGGGTGGTGCTGGGCCTGCTGAAGGAC | |
| | TATTTCAAGGCCAATAACAAGAACACCAAGGTTTTCACAATCAGAGGA | |
| | CAGATCACCAGCCAGTTTCGGAAGAGCAGAAATCTGGTGAAGAGCCG | |
| | GGATTGCTTCTACCACCACATCGTGGATGCCCTGATCATCGCTTACTT | |
| | CCGGAAGTTCAACTACATTAACGAGCTTATGCAGTACAACTTCAGTTA | |
| | CAAAGTGAAGATCGATGAGGAGACAGGCGAACTGATCCAGTATAATG | |
| | ATACCCTGTTCAGCACCAAGGATGACGATTTTACAATCCAACTGAGAA | |
| | AACTTAACGACCTGAACAAGTACAGTGAGGATATCAAGATCAGCCAC | |
| | AAGGTGGATAGAAAGCCTAATAGACAGTTTTCTGACGAGACAATCTAC | |
| | AGCGCCAGAAAGATCAACAACGACTACATGAAGATCAGCAAGTACAA | |
| | GGACATCTATGGCGAGGAGGGCAAGAAGTTCGCTAAGGTTATCAAGG | |
| | ACAACCTGCAGAAGGAGAAGGACCTGGACAAGTTTCTGATGAAGAAG | |
| | GTAGATCCTGAAACCTTCAACCTGCTGGTGTCCATCGTGATTAACACC | |
| | ACTTGCGGCGAGAAGGAGAACCCTTTCGTGAAGTACTGTGAAGAGAA | |
| | CAACATCGACTTCATCCGGAAGAAGAGCAAGAACGGGAACGGCCCTA | |
| | TCATCAGAAGCATCAGATACTACGACGGCAAAGTGAACAGCCACCTG | |
| | GACATCTCTCAGAAGTACGTGAACCTCTCTGACGACAAGAAGGTGGT | |
| | GCTGCTGCAGATCAGCCCCTACCGGACCGACTTCTACCTGACCAAAG | |
| | AGGGCAACTACAAATTCCTGACCATTAAGTACGCCAACATCAAGGAAA | |
| | ATCACAACGGCTACTACATCCCTAAGGACGTGTACGAGAGCCTGAAG | |
| | AAGTCTAAGAAGATCAACGACAGCGATGAGTTCCTGTTTTCTCTGTAC | |
| | AGAAATGACTACCTCAGGATCACTACAAACGACCCTACAGAGAACGC | |
| | TCCTATGATCTATAGATTCGTGGGCACCAACAACGACCTGGAAAACAA | |
| | GGTTGAGGTCAAACCTATCTATTGCGACAAAAATGAAGAGAACCGGA | |
| | TCATGAAAACCATCGGCAAGAAGGTGACCAAGATCGAGAAGTTCGAC | |
| | GTGGATGTGCTGGGAAACAAGATCGCCAAAAAGAGAGACAAGTGTCT | |
| | GCAGCTGGAGCTG | |
| AIWM Type II | ATGATCCCCAATCCCCTGCTGGGCCTGGACAAGCGGACTGCCGACG | 30 |
| Cas | GCAGTGAGTTCGAGTCTCCCAAGAAAAAGAGGAAAGTGggatccGCTAA | |
| mammalian | GTACATCCTGGGACTGGACATGGGCATCACAAGCGTGGGCTGGGGC | |
| expression | CTGATCGATTACGACAGCGGCAAGATCGTGGACAAGGGCGTGCGGC | |
| construct | TGTTTAAGGAGGCCACAGCCGAGGACAACCTGAAGAGAAGGACCCA | |
| (includes N- | CAGAGGCTCCCGGAGACTGAAGCGGCGGAGACAGCAAAGAATTATA | |
| terminal SV5 | GAGCTGAAGCGCCTGCTGAAAAAAGAAAATATCCTGACAGATGACTA | |
| tag and NLS | CACCCCTCTGAACAATGTGTACTATCTGAGGTGTAAAGGACTCAACG | |
| and C- | AGAAACTGACAAACGCCGAGCTGGTGTCCGTCCTGCTTCATATCGCC | |
| terminal NLS) | AAGATTCGGGGCAGCAGCCTGGAAGTGGTCGAAGATGACGAAAAAA | |
| (nt) | AGGCCAACGAAGATAAGAGCAGCAAGGCCAACCTGTCCGTGAATGAA | |
| | ACCAAGCTGAGAACAAGCAAAGAAGAGATCTGCGAGAAGCAATACAA | |
| | AGACCTGATCGAGAACGGCAAGGTCAGAGGAATCAACAACATCTATA | |
| | AGAGCTCTAGCTATGAAAGCGAGCTGAAGAAAATTCTGGCCAATCAG | |
| | GGCCTGAGCAAGGAGGTGAACGATAAGATCATCAACATCATTTTCAG | |
| | ACGGCGGGACTTCAACGAAGGCCCCGGCAGCATGGAGAGCCCAACA | |
| | ATCTACGGCAGGTTCATCCCACACGAGAACGGCGAAATCGAAATCAT | |
| | TCCTTCTATGATCGAAAAGATGCAGGGCAAGTGCAGCATCTACCAGA | |
| | ACGAACCTAGAGCCGCGAAGATGAGCTTCACCGCCGATCTGTTCAAT | |
| | TTCCTGAACGACCTGAACAATTTGGAGATCCAGAGAGCCGACAACTA | |
| | CGACAACTACATCACCCCTGACGAGAAGAAACTGATCATCAACGAGT | |
| | ACATCATTAAGAAGGGCAAGATCACCATCCAGAAGCTGTGCGACGTG | |
| | CTGCAAGTGAAGAAGGAGAACATCACCGGATTCAGAGTGAACAAGGG | |
| | CGAACCCATCCTGACCGAGTTCAAGGGATATCAGAAGGTGCTGAAAG | |
| | CTCTGAACAGAATCATTGATACCGAGATCGTGGATGCCGTGATCGAT | |
| | GTGCTGACCAAATACAAGGACGTGGAAAAACGGAAAGAGGAAATCAA | |
| | AAAGATCAACAATGCTGAGTTCAGCGAGGAAGATATCGTGAAGCTGA | |
| | GCGAGATCACAGGCATCCAGCAGTACCACAGCCTGTCCTACAAGGC | |
| | CATGAAGGTGATCATCCCTGAGCTGTATGAGAACCCCGAGAACCAGA | |
| | TGGAAGTGATCACCAGACTCAATCTGGTGAAAACCAGCATCGACGAC | |
| | CTGAAGGGCAGAACCACCATCCCCAGCTACTCCGACCGGATCTACAG | |
| | CCCTGTGGCCAAAAGAGTGCACAACGAGACAATCAAGGTGGTGAAC | |
| | GCAGTGATCAAGAAGTACGGCGACCTTGACAGCATCGTGATCGAGAT | |
| | GGCCAGAGACAGAAACACCGCCGAAGAAAGAAAGAACATCGAACAG | |
| | GCCCAGAAGCGGAACCAGGAGAATAGAGAGAAAGCTAAAGAAATCCT | |
| | GGACGTAAACTACTCTTTTTCAAAGCTGGATTCTAAGCTGGTCCAGAA | |
| | AATCATGCTGTACAATCAACAAGACGGAAAGTCCATGTACAGCGGCA | |
| | AGCCCATCAATCTGAACGTCCTGATCAGCGACCCTAACGCCTACGAG | |
| | ATCGACCACATCATCCCCCTGTCTATCAGCCTGGACGACAGCATCGC | |
| | CAACAAAGTGCTGGTTTACAGAAGCGAGAACCAGCAGAAGCTGAACA | |
| | ACACACCCCTGCAGTACCTGAGAAGCGGCAATAGCAACGGCTGGAG | |
| | CGTGGACGAGTTCAGAGAAGTGGTGATCAAGATGTACAACGACAAAA | |
| | AAATTTCCCTCAAAAAACTGCAGAACCTGCTGTGCGAGAAGGACATC | |
| | ACCAAGCAGGACGTGCGAAAGGAATTTATCGAGCGGAACCTGGTCG | |
| | ATACAAGATACGCCAGCCGGGTGGTGCTGGGCCTGCTGAAGGACTA | |
| | TTTCAAGGCCAATAACAAGAACACCAAGGTTTTCACAATCAGAGGACA | |
| | GATCACCAGCCAGTTTCGGAAGAGCAGAAATCTGGTGAAGAGCCGG | |
| | GATTGCTTCTACCACCACATCGTGGATGCCCTGATCATCGCTTACTTC | |
| | CGGAAGTTCAACTACATTAACGAGCTTATGCAGTACAACTTCAGTTAC | |
| | AAAGTGAAGATCGATGAGGAGACAGGCGAACTGATCCAGTATAATGA | |
| | TACCCTGTTCAGCACCAAGGATGACGATTTTACAATCCAACTGAGAAA | |
| | ACTTAACGACCTGAACAAGTACAGTGAGGATATCAAGATCAGCCACA | |
| | AGGTGGATAGAAAGCCTAATAGACAGTTTTCTGACGAGACAATCTACA | |
| | GCGCCAGAAAGATCAACAACGACTACATGAAGATCAGCAAGTACAAG | |
| | GACATCTATGGCGAGGAGGGCAAGAAGTTCGCTAAGGTTATCAAGGA | |
| | CAACCTGCAGAAGGAGAAGGACCTGGACAAGTTTCTGATGAAGAAGG | |
| | TAGATCCTGAAACCTTCAACCTGCTGGTGTCCATCGTGATTAACACCA | |
| | CTTGCGGCGAGAAGGAGAACCCTTTCGTGAAGTACTGTGAAGAGAAC | |
| | AACATCGACTTCATCCGGAAGAAGAGCAAGAACGGGAACGGCCCTAT | |
| | CATCAGAAGCATCAGATACTACGACGGCAAAGTGAACAGCCACCTGG | |
| | ACATCTCTCAGAAGTACGTGAACCTCTCTGACGACAAGAAGGTGGTG | |
| | CTGCTGCAGATCAGCCCCTACCGGACCGACTTCTACCTGACCAAAGA | |
| | GGGCAACTACAAATTCCTGACCATTAAGTACGCCAACATCAAGGAAAA | |
| | TCACAACGGCTACTACATCCCTAAGGACGTGTACGAGAGCCTGAAGA | |
| | AGTCTAAGAAGATCAACGACAGCGATGAGTTCCTGTTTTCTCTGTACA | |
| | GAAATGACTACCTCAGGATCACTACAAACGACCCTACAGAGAACGCT | |
| | CCTATGATCTATAGATTCGTGGGCACCAACAACGACCTGGAAAACAA | |
| | GGTTGAGGTCAAACCTATCTATTGCGACAAAAATGAAGAGAACCGGA | |
| | TCATGAAAACCATCGGCAAGAAGGTGACCAAGATCGAGAAGTTCGAC | |
| | GTGGATGTGCTGGGAAACAAGATCGCCAAAAAGAGAGACAAGTGTCT | |
| | GCAGCTGGAGCTGtctagaAAGCGGACAGCAGACGGCTCCGAATTTGA | |
| | AAGCCCTAAGAAAAAGAGAAAGGTGTGA | |

In some embodiments an AIWM Type II Cas protein comprises an amino acid sequence of SEQ ID NO:25, SEQ ID NO:26 or SEQ ID NO:27. In some embodiments, a AIWM Type II Cas protein has nickase activity, for example resulting from one or more amino acid substitutions relative to the sequence of SEQ ID NO:25, SEQ ID NO:26 or SEQ ID NO:27. In some embodiments, the one or more amino acid substitutions providing nickase activity is a D9A substitution, wherein the position of the D9A substitution is defined with respect to the amino acid numbering of SEQ ID NO:26. In some embodiments, the one or more amino acid substitutions providing nickase activity is a N590A substitution, wherein the position of the N590A substitution is defined with respect to the amino acid numbering of SEQ ID NO:26. In some embodiments, an AIWM Type II Cas protein is catalytically inactive, for example due to both a D9A substitution and a N590A substitution, where the positions of the substitutions are defined with respect to the amino acid numbering of SEQ ID NO:26.

### 6.2.1.6. AIWR Type II Cas Proteins

In one aspect, the disclosure provides AIWR Type II Cas proteins. AIWR Type II Cas proteins can be further classified as Type II Cas proteins The AIWR Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:31. In some embodiments, the AIWR Type II Cas proteins comprise an amino acid sequence that is at least 95%, at least 96%, at least 97%, at least 98%, or at least 99% identical to SEQ ID NO:31. In some embodiments, a AIWR Type II Cas protein comprises an amino acid sequence that is identical to SEQ ID NO:31.

Exemplary AIWR Type II Cas protein sequences and nucleotide sequences encoding exemplary AIWR Type II Cas proteins are set forth in Table 1F.

TABLE 1F
AIWR Type II Cas Sequences

| | | SEQ ID |
|---|---|---|
| Name | Sequence | NO |
| AIWR Type II | AKYILGLDMGITSVGWGLIDYDSGKIVDKGVRLFKEATAEDNLNRRTHRG | 31 |
| Cas amino | SRRLKRRRQQRIIELKRLLKKENILTDDYTPLNNVYYLRCKGLNEKLTNAE | |
| acid | LVSVLLHIAKIRGSSLEVVEDDEKKANEDKSSKANLSVNETKLRTSKEEIC | |
| sequence | EKQYKDLIENGKVRGINNIYKSSSYESELKKILDNQGLSKEVNDKIINIIFRR | |
| (without N- | RDFNEGPGSIESPTIYGRFIPHENGEIEIIPSMIEKMQGKCSIYQDEPRAPK | |
| terminal | MSFTADLFNFLNDLNNLEIQRADNYDNYITPDEKKLIINEYIIKKGKITIQKL | |
| valine) | CDVLQVKKENITGFRVNKGEPILTEFKGYQKVLKALNRIIDTEIVDAVIDVL | |
| | TKYKDVEKRKEEIKKINNAEFSEEDIVKLSEITGIQQYHSLSYKAMKVIIPEL | |
| | YENPENQMEVITRLNLVKTSIDDLKGRTTIPSYSDRIYSPVAKRVHNETIK | |
| | WVNAVIKKYGDLDSIVIEMARDRNTAEERKNIEQAQKRNQENREKAKEIL | |
| | DANHSFSKLDSKLVQKIMLYNQQDGKSMYSGKPINLNVLISDPNAYEIDHI | |
| | IPLSISLDDSIANKVLVYRSENQQKLNNTPLQYLRSGNSNGWSVDEFREV | |
| | VIKMYNDKKISLKKLQNLLCEKDITKQDVRKEFIERNLVDTRYASRVVLGL | |
| | LKDYFKANNKNTKVFTIRGQITSQFRKSRNLVKSRDCFYHHIVDALIIAYFR | |
| | KFNYINELMQYNFNYKGKIDEETGELIQYNDTLFSTKDDDFTIQLRKLNDL | |
| | NKYSEDIKISHKVDRKPNRKFSDETIYSARKIDNDYMKISKYKDIYGEEGK | |
| | KFAKVIKDNLQKEKDLDKFLMKKVDPETFNLLVSIVINTTCGEKENPFVKY | |
| | CEENNIDFIRKKSKNGNGPIIRSIRYYDGKVNSHLDISQKYSNLPNDKKVV | |
| | LLQISPYRTDFYLTKEGNYKFLTIRYANIRENHNGYYIPKEVYESLKKSKKI | |
| | NDSDEFLFSLYQNDYLRITTNDPTENVPMIYRFVGTNNDLANKVEVKPIY | |
| | CNKIDKPQIMKTIGKKVTKIEKFDVDVLGNKIAKKRDKCLQLEL | |
| AIWR Type II | VAKYILGLDMGITSVGWGLIDYDSGKIVDKGVRLFKEATAEDNLNRRTHR | 32 |
| Cas amino | GSRRLKRRRQQRIIELKRLLKKENILTDDYTPLNNVYYLRCKGLNEKLTNA | |
| acid | ELVSVLLHIAKIRGSSLEVVEDDEKKANEDKSSKANLSVNETKLRTSKEEI | |
| sequence | CEKQYKDLIENGKVRGINNIYKSSSYESELKKILDNQGLSKEVNDKIINIIFR | |
| | RRDFNEGPGSIESPTIYGRFIPHENGEIEIIPSMIEKMQGKCSIYQDEPRAP | |
| | KMSFTADLFNFLNDLNNLEIQRADNYDNYITPDEKKLIINEYIIKKGKITIQKL | |
| | CDVLQVKKENITGFRVNKGEPILTEFKGYQKVLKALNRIIDTEIVDAVIDVL | |
| | TKYKDVEKRKEEIKKINNAEFSEEDIVKLSEITGIQQYHSLSYKAMKVIIPEL | |
| | YENPENQMEVITRLNLVKTSIDDLKGRTTIPSYSDRIYSPVAKRVHNETIK | |
| | WVNAVIKKYGDLDSIVIEMARDRNTAEERKNIEQAQKRNQENREKAKEIL | |
| | DANHSFSKLDSKLVQKIMLYNQQDGKSMYSGKPINLNVLISDPNAYEIDHI | |
| | IPLSISLDDSIANKVLVYRSENQQKLNNTPLQYLRSGNSNGWSVDEFREV | |
| | VIKMYNDKKISLKKLQNLLCEKDITKQDVRKEFIERNLVDTRYASRVVLGL | |
| | LKDYFKANNKNTKVFTIRGQITSQFRKSRNLVKSRDCFYHHIVDALIIAYFR | |
| | KFNYINELMQYNFNYKGKIDEETGELIQYNDTLFSTKDDDFTIQLRKLNDL | |
| | NKYSEDIKISHKVDRKPNRKFSDETIYSARKIDNDYMKISKYKDIYGEEGK | |
| | KFAKVIKDNLQKEKDLDKFLMKKVDPETFNLLVSIVINTTCGEKENPFVKY | |
| | CEENNIDFIRKKSKNGNGPIIRSIRYYDGKVNSHLDISQKYSNLPNDKKVV | |
| | LLQISPYRTDFYLTKEGNYKFLTIRYANIRENHNGYYIPKEVYESLKKSKKI | |
| | NDSDEFLFSLYQNDYLRITTNDPTENVPMIYRFVGTNNDLANKVEVKPIY | |
| | CNKIDKPQIMKTIGKKVTKIEKFDVDVLGNKIAKKRDKCLQLEL | |
| | MIPNPLLGLDKRTADGSEFESPKKKRKVGSAKYILGLDMGITSVGWGLID | 33 |
| | YDSGKIVDKGVRLFKEATAEDNLNRRTHRGSRRLKRRRQQRIIELKRLLK | |
| | KENILTDDYTPLNNVYYLRCKGLNEKLTNAELVSVLLHIAKIRGSSLEVVE | |
| | DDEKKANEDKSSKANLSVNETKLRTSKEEICEKQYKDLIENGKVRGINNIY | |
| | KSSSYESELKKILDNQGLSKEVNDKIINIIFRRRDFNEGPGSIESPTIYGRFI | |
| | PHENGEIEIIPSMIEKMQGKCSIYQDEPRAPKMSFTADLFNFLNDLNNLEI | |
| | QRADNYDNYITPDEKKLIINEYIIKKGKITIQKLCDVLQVKKENITGFRVNKG | |
| | EPILTEFKGYQKVLKALNRIIDTEIVDAVIDVLTKYKDVEKRKEEIKKINNAE | |
| | FSEEDIVKLSEITGIQQYHSLSYKAMKVIIPELYENPENQMEVITRLNLVKT | |
| | SIDDLKGRTTIPSYSDRIYSPVAKRVHNETIKVVNAVIKKYGDLDSIVIEMA | |
| | RDRNTAEERKNIEQAQKRNQENREKAKEILDANHSFSKLDSKLVQKIMLY | |
| | NQQDGKSMYSGKPINLNVLISDPNAYEIDHIIPLSISLDDSIANKVLVYRSE | |
| | NQQKLNNTPLQYLRSGNSNGWSVDEFREVVIKMYNDKKISLKKLQNLLC | |
| | EKDITKQDVRKEFIERNLVDTRYASRVVLGLLKDYFKANNKNTKVFTIRG | |
| | QITSQFRKSRNLVKSRDCFYHHIVDALIIAYFRKFNYINELMQYNFNYKGKI | |
| | DEETGELIQYNDTLFSTKDDDFTIQLRKLNDLNKYSEDIKISHKVDRKPNR | |
| | KFSDETIYSARKIDNDYMKISKYKDIYGEEGKKFAKVIKDNLQKEKDLDKF | |
| | LMKKVDPETFNLLVSIVINTTCGEKENPFVKYCEENNIDFIRKKSKNGNGP | |
| | IIRSIRYYDGKVNSHLDISQKYSNLPNDKKVVLLQISPYRTDFYLTKEGNY | |
| | KFLTIRYANIRENHNGYYIPKEVYESLKKSKKINDSDEFLFSLYQNDYLRIT | |
| | TNDPTENVPMIYRFVGTNNDLANKVEVKPIYCNKIDKPQIMKTIGKKVTKI | |
| | EKFDVDVLGNKIAKKRDKCLQLELSRKRTADGSEFESPKKKRKV | |
| AIWR Type II | GTGGCAAAATATATATTAGGACTAGATATGGGAATTACTTCGGTAGGT | 34 |
| Cas coding | TGGGGCCTAATTGACTATGATTCTGGGAAAATAGTTGATAAAGGCGTT | |
| sequence (nt) | AGACTTTTTAAAGAAGCAACTGCGGAAGATAATTTAAACAGACGTACT | |
| (not codon | CATCGTGGTTCAAGAAGGTTAAAAAGACGTAGACAACAAAGAATAATT | |
| optimized) | GAATTAAAACGATTATTAAAAAAAGAAAACATTCTTACAGATGATTATA | |
| | CTCCTTTAAATAATGTTTACTATCTTAGATGTAAAGGCTTAAACGAAAA | |
| | ATTAACAAATGCTGAACTTGTATCAGTATTATTACATATAGCAAAAATA | |
| | AGAGGAAGTTCACTAGAAGTAGTTGAAGATGATGAAAAAAAGGCTAAT | |
| | GAGGATAAAAGTAGTAAAGCCAATTTATCAGTAAATGAAACAAAATTA | |
| | AGAACTTCAAAAGAAGAAATTTGTGAAAAACAGTATAAAGATCTAATC | |
| | GAAAATGGTAAAGTGCGTGGAATAAATAATATTTATAAAAGTTCTTCAT | |
| | ATGAGTCAGAACTCAAAAAAATTTTAGATAATCAAGGCTTGTCAAAAG | |
| | AAGTTAATGATAAAATAATAAATATAATTTTTAGAAGACGTGACTTTAAT | |
| | GAAGGACCAGGAAGCATAGAATCGCCTACTATATATGGTAGATTTATA | |
| | CCACATGAAAATGGTGAGATAGAAATTATACCTAGTATGATTGAAAAA | |
| | ATGCAGGGAAAATGTTCTATATATCAAGACGAGCCTCGTGCACCGAA | |
| | AATGTCATTTACAGCTGATTTGTTTAATTTCTTAAACGATTTAAACAATT | |
| | TAGAAATACAGCGTGCTGATAACTATGATAATTATATTACCCCAGACG | |
| | AAAAAAAACTAATAATAAATGAGTATATCATCAAAAAAGGTAAAATTAC | |
| | AATTCAAAAATTATGTGATGTACTACAGGTAAAAAAAGAAAATATTACT | |
| | GGCTTTAGAGTTAACAAAGGTGAACCAATACTCACTGAATTTAAAGGC | |
| | TATCAAAAAGTATTAAAAGCTTTAAATAGAATAATTGATACTGAAATTG | |
| | TTGATGCGGTTATTGACGTTTTAACAAAATATAAAGATGTAGAAAAAAG | |
| | AAAAGAAGAAATTAAAAAAATAAACAATGCAGAATTTAGTGAAGAAGA | |
| | CATTGTAAAATTATCAGAAATAACTGGTATACAACAATATCATTCATTG | |
| | TCTTATAAAGCAATGAAAGTAATAATACCTGAATTATATGAAAATCCTG | |
| | AAAATCAAATGGAAGTAATTACAAGATTAAATCTAGTGAAAACTTCAAT | |
| | AGATGATTTGAAAGGAAGAACAACAATACCTTCATATAGCGACAGAAT | |
| | ATATTCCCCTGTAGCTAAACGTGTACATAATGAGACAATCAAAGTAGT | |
| | AAATGCGGTAATAAAAAAATATGGAGATTTAGATAGTATTGTTATAGAA | |
| | ATGGCAAGGGATAGAAATACTGCTGAAGAAAGGAAAAATATTGAACAA | |
| | GCCCAAAAACGAAATCAAGAAAATAGAGAAAAAGCAAAAGAGATACTA | |
| | GATGCTAATCATAGTTTTTCGAAACTTGATTCAAAATTAGTTCAAAAGA | |
| | TCATGCTTTATAATCAACAAGATGGTAAAAGTATGTATAGTGGAAAAC | |
| | CAATAAACTTAAATGTTTTAATAAGTGACCCTAATGCATACGAGATAGA | |
| | TCATATCATACCACTATCAATTTCGCTTGATGATTCAATTGCCAATAAA | |
| | GTTCTGGTGTATAGAAGTGAAAACCAACAAAAATTAAATAATACACCA | |
| | CTTCAATACTTACGTAGTGGTAATTCAAATGGTTGGAGCGTCGACGAA | |
| | TTCCGTGAAGTTGTAATAAAAATGTATAATGATAAAAAAATATCACTAA | |
| | AAAAATTACAAAACTTATTATGTGAAAAAGATATAACAAAACAAGATGT | |
| | AAGAAAAGAATTTATTGAAAGAAATTTAGTTGACACAAGATATGCAAGT | |
| | AGGGTAGTTTTGGGACTTCTAAAAGATTATTTCAAAGCCAACAATAAA | |
| | AATACTAAAGTATTTACAATAAGAGGCCAAATAACATCACAATTCAGAA | |
| | AAAGTAGAAACTTGGTGAAAAGTAGAGATTGCTTTTATCATCATATAGT | |
| | AGATGCATTAATAATTGCATATTTTAGAAAATTCAATTACATTAATGAAT | |
| | TAATGCAATACAATTTTAATTATAAAGGGAAAATTGATGAAGAAACAGG | |
| | AGAATTAATCCAATATAATGATACGTTGTTTTCAACGAAGGATGATGAT | |
| | TTCACAATACAATTAAGAAAACTAAATGACCTTAATAAATATAGTGAAG | |
| | ATATCAAAATTTCGCATAAAGTTGATAGAAAGCCTAATAGAAAGTTTAG | |
| | TGACGAAACAATCTATTCTGCGAGAAAAATCGATAACGATTATATGAA | |
| | AATTTCAAAATATAAAGATATTTATGGTGAAGAAGGTAAAAAATTTGCA | |
| | AAAGTAATTAAAGATAATCTTCAAAAAGAAAAAGACTTAGATAAATTCT | |
| | TAATGAAGAAAGTAGATCCCGAAACTTTTAATTTATTAGTTAGCATTGT | |
| | TATAAATACAACATGTGGTGAAAAAGAAAACCCTTTTGTTAAATATTGT | |
| | GAAGAAAACAATATAGATTTTATTAGAAAAAAATCTAAAAATGGTAATG | |
| | GTCCGATTATTAGGTCTATTAGATATTATGATGGTAAAGTAAATAGTCA | |
| | CCTTGATATCTCACAAAAATATTCAAACTTACCAAATGACAAAAAGGTT | |
| | GTACTTTTACAAATTTCACCATATAGAACTGATTTTTATTTAACTAAAGA | |
| | AGGAAACTATAAATTTCTAACAATTAGATATGCTAATATCAGAGAAAAC | |
| | CATAATGGATATTATATACCAAAAGAAGTTTACGAATCATTAAAGAAAT | |
| | CAAAGAAAATAAATGATAGTGATGAATTCTTATTTTCGCTATATCAAAA | |
| | TGATTACCTAAGAATTACAACAAATGATCCTACTGAAAATGTTCCTATG | |
| | ATTTATCGATTTGTAGGTACAAATAATGATTTAGCAAACAAAGTTGAAG | |
| | TAAAACCAATTTATTGTAATAAGATAGATAAACCACAAATTATGAAAAC | |
| | CATAGGTAAAAAAGTAACTAAAATTGAAAAGTTTGATGTTGATGTACTA | |
| | GGTAATAAAATTGCTAAAAAAAGAGATAAATGCTTGCAATTAGAGCTG | |
| | TAG | |
| AIWR Type II | GCCAAGTACATCCTGGGCCTGGACATGGGCATCACCAGCGTGGGCT | 35 |
| Cas coding | GGGGCCTGATTGATTACGACAGCGGCAAGATCGTGGACAAGGGCGT | |
| sequence (nt) | GCGGCTGTTTAAGGAGGCCACCGCCGAGGACAACCTGAACAGAAGG | |
| (human | ACCCACAGAGGCAGCAGACGGCTGAAGCGGCGGAGACAGCAGAGA | |
| codon- | ATCATCGAACTAAAGCGGCTGCTGAAGAAGGAGAACATCCTGACCGA | |
| optimized) | CGATTACACCCCTCTGAATAACGTGTACTACCTGCGGTGCAAAGGCC | |
| | TGAACGAAAAGCTGACCAACGCCGAACTCGTGTCTGTTCTGCTGCAC | |
| | ATCGCCAAGATTCGGGGCTCTTCCCTTGAGGTGGTAGAGGACGATGA | |
| | GAAGAAAGCAAACGAGGATAAAAGCTCCAAGGCCAATCTGAGCGTGA | |
| | ATGAAACCAAGCTGCGGACAAGCAAGGAAGAAATCTGCGAGAAGCA | |
| | GTACAAGGACCTGATCGAAAACGGCAAGGTGCGGGGAATCAATAACA | |
| | TCTACAAGTCCTCTAGCTACGAATCTGAACTGAAGAAGATTCTGGACA | |
| | ATCAGGGCCTGTCCAAGGAAGTGAACGACAAAATCATAAACATTATCT | |
| | TCAGACGGAGAGACTTCAACGAGGGCCCTGGCAGCATTGAGAGCCC | |
| | TACCATCTATGGAAGATTCATCCCCCACGAGAACGGCGAGATCGAGA | |
| | TAATCCCCAGCATGATCGAGAAAATGCAGGGAAAATGCAGCATCTAC | |
| | CAGGATGAGCCTCGGGCCCCTAAGATGAGCTTCACCGCTGATTTGTT | |
| | TAACTTCCTGAACGATCTGAATAACCTGGAAATCCAGAGAGCCGACA | |
| | ACTACGACAATTACATCACTCCAGACGAGAAGAAGCTGATCATCAAC | |
| | GAGTACATCATCAAGAAGGGCAAGATCACCATTCAGAAACTGTGCGA | |
| | CGTGCTGCAAGTGAAGAAGGAGAACATCACAGGCTTCCGCGTGAACA | |
| | AGGGAGAACCCATTCTGACCGAGTTCAAGGGCTATCAGAAAGTGCTG | |
| | AAGGCTCTGAACAGAATCATCGACACCGAGATCGTGGATGCCGTGAT | |
| | CGACGTGCTGACAAAGTACAAGGATGTGGAAAAAAGAAAAGAGGAGA | |
| | TCAAGAAGATTAACAACGCTGAGTTCAGCGAGGAGGACATCGTGAAA | |
| | CTGAGCGAGATCACAGGAATCCAGCAGTACCACAGCCTGAGCTACAA | |
| | GGCTATGAAAGTGATCATCCCAGAGCTGTACGAGAACCCTGAGAATC | |
| | AGATGGAAGTGATCACAAGACTGAATCTGGTGAAAACCAGTATCGAT | |
| | GACCTGAAAGGCAGAACCACCATCCCTAGCTACAGCGACCGGATCTA | |
| | CAGCCCTGTGGCCAAGAGAGTGCATAACGAAACAATCAAAGTGGTGA | |
| | ACGCCGTGATCAAGAAATACGGAGATCTGGACAGCATCGTGATCGAG | |
| | ATGGCCAGAGATAGAAATACCGCCGAGGAGAGAAAGAACATCGAGC | |
| | AGGCCCAGAAAAGAAACCAGGAGAACAGAGAAAAAGCCAAGGAAATC | |
| | CTGGATGCTAACCACTCTTTCAGCAAATTGGACTCTAAACTGGTCCAG | |
| | AAAATCATGCTGTACAACCAGCAAGACGGCAAGAGCATGTACAGCGG | |
| | CAAACCTATCAATCTGAACGTGCTGATCAGCGATCCCAACGCTTACG | |
| | AGATCGATCACATCATCCCACTGTCCATTTCCCTGGACGACAGCATC | |
| | GCCAACAAAGTTCTGGTGTACAGAAGCGAAAATCAACAGAAGTTGAA | |
| | TAATACCCCTCTGCAGTACCTGAGATCTGGCAACAGCAACGGCTGGA | |
| | GCGTTGACGAGTTCCGGGAAGTCGTCATCAAGATGTACAACGACAAG | |
| | AAGATCTCCCTCAAGAAGCTGCAGAACCTGCTGTGTGAAAAGGACAT | |
| | CACAAAGCAAGACGTGCGGAAGGAATTTATCGAGCGGAATCTGGTGG | |
| | ACACAAGATACGCCAGCAGGGTGGTTCTGGGTCTGCTGAAGGACTAC | |
| | TTCAAGGCCAACAACAAGAACACCAAGGTGTTCACCATCCGGGGCCA | |
| | GATCACCAGCCAGTTCAGAAAGAGCAGAAACCTGGTGAAGAGCAGA | |
| | GATTGCTTCTACCACCACATCGTGGACGCCCTGATCATCGCCTACTT | |
| | CAGAAAGTTCAACTACATCAACGAGCTGATGCAGTACAACTTCAATTA | |
| | TAAGGGCAAGATCGATGAAGAAACCGGCGAGCTGATCCAGTACAACG | |
| | ACACCCTGTTCTCTACAAAGGACGACGATTTTACAATCCAGCTGAGAA | |
| | AGCTGAATGACTTGAACAAGTACAGCGAGGACATCAAAATCTCTCACA | |
| | AGGTGGACAGAAAGCCTAACCGGAAGTTCAGCGATGAGACTATCTAC | |
| | AGCGCCAGAAAAATTGATAACGACTACATGAAGATCTCTAAGTATAAG | |
| | GATATCTACGGCGAAGAAGGCAAGAAGTTTGCCAAGGTGATCAAAGA | |
| | TAATCTGCAGAAGGAAAAGGACCTGGATAAGTTCCTGATGAAGAAAG | |
| | TGGACCCTGAAACCTTCAACCTGCTCGTGTCCATCGTGATCAACACC | |
| | ACCTGTGGCGAGAAGGAGAATCCTTTCGTGAAGTACTGTGAAGAGAA | |
| | CAATATCGACTTTATCCGAAAAAAAAGTAAAAACGGCAACGGCCCCAT | |
| | CATCCGGTCTATCAGATACTATGACGGCAAGGTCAACAGCCACCTGG | |
| | ACATCTCACAGAAGTATTCTAACCTGCCTAACGACAAAAAGGTGGTGC | |
| | TGCTGCAAATCTCCCCGTACAGAACAGATTTTTACCTGACAAAGGAG | |
| | GGAAATTACAAGTTCCTGACCATCAGATACGCCAACATCAGAGAGAA | |
| | CCATAATGGATACTACATCCCCAAGGAAGTGTACGAGAGCCTGAAGA | |
| | AAAGCAAGAAGATCAACGATTCAGACGAGTTTCTGTTCTCTCTGTACC | |
| | AAAACGACTACCTGAGAATCACAACAAACGACCCCACCGAGAACGTG | |
| | CCTATGATCTATAGATTCGTGGGTACAAACAACGATCTGGCGAACAA | |
| | GGTTGAGGTGAAGCCTATCTATTGCAACAAGATCGACAAGCCTCAGA | |
| | TCATGAAAACCATAGGGAAGAAGGTGACCAAGATAGAGAAATTCGAC | |
| | GTAGACGTGCTTGGCAACAAGATCGCCAAAAAGCGGGACAAGTGCCT | |
| | GCAGCTGGAGCTG | |
| AIWR Type II | ATGATCCCCAATCCCCTGCTGGGCCTGGACAAGCGGACTGCCGACG | 36 |
| Cas | GCAGTGAGTTCGAGTCTCCCAAGAAAAAGAGGAAAGTGggatccGCCA | |
| mammalian | AGTACATCCTGGGCCTGGACATGGGCATCACCAGCGTGGGCTGGGG | |
| expression | CCTGATTGATTACGACAGCGGCAAGATCGTGGACAAGGGCGTGCGG | |
| construct | CTGTTTAAGGAGGCCACCGCCGAGGACAACCTGAACAGAAGGACCC | |
| (includes N- | ACAGAGGCAGCAGACGGCTGAAGCGGCGGAGACAGCAGAGAATCAT | |
| terminal SV5 | CGAACTAAAGCGGCTGCTGAAGAAGGAGAACATCCTGACCGACGATT | |
| tag and NLS | ACACCCCTCTGAATAACGTGTACTACCTGCGGTGCAAAGGCCTGAAC | |
| and C- | GAAAAGCTGACCAACGCCGAACTCGTGTCTGTTCTGCTGCACATCGC | |
| terminal NLS) | CAAGATTCGGGGCTCTTCCCTTGAGGTGGTAGAGGACGATGAGAAGA | |
| (nt) | AAGCAAACGAGGATAAAAGCTCCAAGGCCAATCTGAGCGTGAATGAA | |
| | ACCAAGCTGCGGACAAGCAAGGAAGAAATCTGCGAGAAGCAGTACAA | |
| | GGACCTGATCGAAAACGGCAAGGTGCGGGGAATCAATAACATCTACA | |
| | AGTCCTCTAGCTACGAATCTGAACTGAAGAAGATTCTGGACAATCAG | |
| | GGCCTGTCCAAGGAAGTGAACGACAAAATCATAAACATTATCTTCAGA | |
| | CGGAGAGACTTCAACGAGGGCCCTGGCAGCATTGAGAGCCCTACCA | |
| | TCTATGGAAGATTCATCCCCCACGAGAACGGCGAGATCGAGATAATC | |
| | CCCAGCATGATCGAGAAAATGCAGGGAAAATGCAGCATCTACCAGGA | |
| | TGAGCCTCGGGCCCCTAAGATGAGCTTCACCGCTGATTTGTTTAACTT | |
| | CCTGAACGATCTGAATAACCTGGAAATCCAGAGAGCCGACAACTACG | |
| | ACAATTACATCACTCCAGACGAGAAGAAGCTGATCATCAACGAGTACA | |
| | TCATCAAGAAGGGCAAGATCACCATTCAGAAACTGTGCGACGTGCTG | |
| | CAAGTGAAGAAGGAGAACATCACAGGCTTCCGCGTGAACAAGGGAG | |
| | AACCCATTCTGACCGAGTTCAAGGGCTATCAGAAAGTGCTGAAGGCT | |
| | CTGAACAGAATCATCGACACCGAGATCGTGGATGCCGTGATCGACGT | |
| | GCTGACAAAGTACAAGGATGTGGAAAAAAGAAAAGAGGAGATCAAGA | |
| | AGATTAACAACGCTGAGTTCAGCGAGGAGGACATCGTGAAACTGAGC | |
| | GAGATCACAGGAATCCAGCAGTACCACAGCCTGAGCTACAAGGCTAT | |
| | GAAAGTGATCATCCCAGAGCTGTACGAGAACCCTGAGAATCAGATGG | |
| | AAGTGATCACAAGACTGAATCTGGTGAAAACCAGTATCGATGACCTG | |
| | AAAGGCAGAACCACCATCCCTAGCTACAGCGACCGGATCTACAGCCC | |
| | TGTGGCCAAGAGAGTGCATAACGAAACAATCAAAGTGGTGAACGCCG | |
| | TGATCAAGAAATACGGAGATCTGGACAGCATCGTGATCGAGATGGCC | |
| | AGAGATAGAAATACCGCCGAGGAGAGAAAGAACATCGAGCAGGCCC | |
| | AGAAAAGAAACCAGGAGAACAGAGAAAAAGCCAAGGAAATCCTGGAT | |
| | GCTAACCACTCTTTCAGCAAATTGGACTCTAAACTGGTCCAGAAAATC | |
| | ATGCTGTACAACCAGCAAGACGGCAAGAGCATGTACAGCGGCAAACC | |
| | TATCAATCTGAACGTGCTGATCAGCGATCCCAACGCTTACGAGATCG | |
| | ATCACATCATCCCACTGTCCATTTCCCTGGACGACAGCATCGCCAAC | |
| | AAAGTTCTGGTGTACAGAAGCGAAAATCAACAGAAGTTGAATAATACC | |
| | CCTCTGCAGTACCTGAGATCTGGCAACAGCAACGGCTGGAGCGTTGA | |
| | CGAGTTCCGGGAAGTCGTCATCAAGATGTACAACGACAAGAAGATCT | |
| | CCCTCAAGAAGCTGCAGAACCTGCTGTGTGAAAAGGACATCACAAAG | |
| | CAAGACGTGCGGAAGGAATTTATCGAGCGGAATCTGGTGGACACAAG | |
| | ATACGCCAGCAGGGTGGTTCTGGGTCTGCTGAAGGACTACTTCAAGG | |
| | CCAACAACAAGAACACCAAGGTGTTCACCATCCGGGGCCAGATCACC | |
| | AGCCAGTTCAGAAAGAGCAGAAACCTGGTGAAGAGCAGAGATTGCTT | |
| | CTACCACCACATCGTGGACGCCCTGATCATCGCCTACTTCAGAAAGT | |
| | TCAACTACATCAACGAGCTGATGCAGTACAACTTCAATTATAAGGGCA | |
| | AGATCGATGAAGAAACCGGCGAGCTGATCCAGTACAACGACACCCTG | |
| | TTCTCTACAAAGGACGACGATTTTACAATCCAGCTGAGAAAGCTGAAT | |
| | GACTTGAACAAGTACAGCGAGGACATCAAAATCTCTCACAAGGTGGA | |
| | CAGAAAGCCTAACCGGAAGTTCAGCGATGAGACTATCTACAGCGCCA | |
| | GAAAAATTGATAACGACTACATGAAGATCTCTAAGTATAAGGATATCT | |
| | ACGGCGAAGAAGGCAAGAAGTTTGCCAAGGTGATCAAAGATAATCTG | |
| | CAGAAGGAAAAGGACCTGGATAAGTTCCTGATGAAGAAAGTGGACCC | |
| | TGAAACCTTCAACCTGCTCGTGTCCATCGTGATCAACACCACCTGTG | |
| | GCGAGAAGGAGAATCCTTTCGTGAAGTACTGTGAAGAGAACAATATC | |
| | GACTTTATCCGAAAAAAAAGTAAAAACGGCAACGGCCCCATCATCCG | |
| | GTCTATCAGATACTATGACGGCAAGGTCAACAGCCACCTGGACATCT | |
| | CACAGAAGTATTCTAACCTGCCTAACGACAAAAAGGTGGTGCTGCTG | |
| | CAAATCTCCCCGTACAGAACAGATTTTTACCTGACAAAGGAGGGAAAT | |
| | TACAAGTTCCTGACCATCAGATACGCCAACATCAGAGAGAACCATAAT | |
| | GGATACTACATCCCCAAGGAAGTGTACGAGAGCCTGAAGAAAAGCAA | |
| | GAAGATCAACGATTCAGACGAGTTTCTGTTCTCTCTGTACCAAAACGA | |
| | CTACCTGAGAATCACAACAAACGACCCCACCGAGAACGTGCCTATGA | |
| | TCTATAGATTCGTGGGTACAAACAACGATCTGGCGAACAAGGTTGAG | |
| | GTGAAGCCTATCTATTGCAACAAGATCGACAAGCCTCAGATCATGAAA | |
| | ACCATAGGGAAGAAGGTGACCAAGATAGAGAAATTCGACGTAGACGT | |
| | GCTTGGCAACAAGATCGCCAAAAAGCGGGACAAGTGCCTGCAGCTG | |
| | GAGCTGtctagaAAGCGGACAGCAGACGGCTCCGAATTTGAAAGCCCTA | |
| | AGAAAAAGAGAAAGGTGTGA | |

In some embodiments an AIWR Type II Cas protein comprises an amino acid sequence of SEQ ID NO:31, SEQ ID NO:32 or SEQ ID NO:33. In some embodiments, an AIWRType II Cas protein has nickase activity, for example resulting from one or more amino acid substitutions relative to the sequence of SEQ ID NO:31, SEQ ID NO:32 or SEQ ID NO:33. In some embodiments, the one or more amino acid substitutions providing nickase activity is a D9A substitution, wherein the position of the D9A substitution is defined with respect to the amino acid numbering of SEQ ID NO:32. In some embodiments, the one or more amino acid substitutions providing nickase activity is a N590A substitution, wherein the position of the N590A substitution is defined with respect to the amino acid numbering of SEQ ID NO:32. In some embodiments, an AIWR Type II Cas protein is catalytically inactive, for example due to both a D9A substitution and a N590A substitution, where the positions of the substitutions are defined with respect to the amino acid numbering of SEQ ID NO:32.

### 6.2.1.7. AIYQ Type II Cas Proteins

In one aspect, the disclosure provides AIYQ Type II Cas proteins. AIYQ Type II Cas proteins can be further classified as Type IIA Cas proteins. The AIYQ Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:37. In some embodiments, the AIYQ Type II Cas proteins comprise an amino acid sequence that is at least 95%, at least 96%, at least 97%, at least 98%, or at least 99% identical to SEQ ID NO:37. In some embodiments, a AIYQ Type II Cas protein comprises an amino acid sequence that is identical to SEQ ID NO:37.

Exemplary AIYQ Type II Cas protein sequences and nucleotide sequences encoding exemplary AIYQ Type II Cas proteins are set forth in Table 1G.

TABLE 1G
AIYQ Type II Cas Sequences

| | | SEQ ID |
|---|---|---|
| Name | Sequence | NO |
| AIYQ Type II | AKYILGLDMGITSVGWGLIDYDSGKIVDKGVRLFKEATAEDNLKRRTHRG | 37 |
| Cas amino | SRRLKRRRQQRIIELKRLLKKENILTDDYTPLNNVYSLRCKGLNEKLTNAE | |
| acid | LVSVLLHIAKIRGSSLEVVEDDEKKANEDKSSKANLSVNETKLRTSKEEIC | |
| sequence | EKQYKDLIENGKVRGINNIYKSSSYESELKKILANQGLSKEVNDKIINIIFRR | |
| (without N- | RDFNEGPGSMESPTIYGRFIPHKNGEVEVIHSMIEKMQGKCSIYQNEPRA | |
| terminal | AKMSFTADLFNFLNDLNNLEIQRADNYDNYITPDEKKLIINEYIIKKGKITIQ | |
| valine) | KLCDVLQVKKENITGFRVNKGEPILTEFKGYQKVLKALNRIIDTEIVDAVID | |
| | VLTMHKDVEKRKEEIKKINNTEFSEEDIVKLSEITGIQQYHSLSYKAMKVII | |
| | PELYENPENQMEVITRLNLVKTSIDDLKGRTTIPSYSDRIYSPVAKRVHNE | |
| | TIKVVNAVIKKYGDLDSIVIEMARDRNTAEEQKNIEQVKKRNQENREKAKE | |
| | IIEVNHSFSKLDSKLVQKIMLYNQQDGKSMYSGKPINLNVLISDREAYEID | |
| | HIIPLSISLDDSIANKVLVYKSENQQKLNNTPLQYLRSGNSNGWSVDEFR | |
| | EVVIKMYNDKKISLKKLQNLLCEKDITKYDVRKGFIERNLVDTRYASRVVL | |
| | GLLKDYFKANNKNTKVFTVRGQITSQFRRAKNLKKDRNYFYHHIVDALIIA | |
| | HFRKFNYISELMQYGFNYKEKIDEETGELIQYNDTLFPKKDDDFTIQLRKL | |
| | NDLNIYSENIKISHKVDRKPNRQFSDETIYSARKIDNDYMKILKYKDIYGEE | |
| | GKKFAKKIKDNLQKENELDIFLMKKVDPETFNLLVSIVKNTICGEKENPFV | |
| | KYCEENNIDFIRKKSKNGNGPIIRSIRYYKKIESDYLDVSNKYINLPYNKKV | |
| | LLDSTKNFRIDFYKSKNGIYKFLVIKYYYIDKINNKYYIPLNIYEKLKKDRCIN | |
| | DSDEFLFSVYKNEYLRITTKDSKKNVPNIYRFIGTTHNTIELKPLYCDEEQE | |
| | KKRNRPTIGKKITKIEKFDVDVLGNKIAKKRDKCLQLEL | |
| AIYQ Type II | VAKYILGLDMGITSVGWGLIDYDSGKIVDKGVRLFKEATAEDNLKRRTHR | 38 |
| Cas amino | GSRRLKRRRQQRIIELKRLLKKENILTDDYTPLNNVYSLRCKGLNEKLTNA | |
| acid | ELVSVLLHIAKIRGSSLEVVEDDEKKANEDKSSKANLSVNETKLRTSKEEI | |
| sequence | CEKQYKDLIENGKVRGINNIYKSSSYESELKKILANQGLSKEVNDKIINIIFR | |
| | RRDFNEGPGSMESPTIYGRFIPHKNGEVEVIHSMIEKMQGKCSIYQNEPR | |
| | AAKMSFTADLFNFLNDLNNLEIQRADNYDNYITPDEKKLIINEYIIKKGKITI | |
| | QKLCDVLQVKKENITGFRVNKGEPILTEFKGYQKVLKALNRIIDTEIVDAVI | |
| | DVLTMHKDVEKRKEEIKKINNTEFSEEDIVKLSEITGIQQYHSLSYKAMKVI | |
| | IPELYENPENQMEVITRLNLVKTSIDDLKGRTTIPSYSDRIYSPVAKRVHNE | |
| | TIKVVNAVIKKYGDLDSIVIEMARDRNTAEEQKNIEQVKKRNQENREKAKE | |
| | IIEVNHSFSKLDSKLVQKIMLYNQQDGKSMYSGKPINLNVLISDREAYEID | |
| | HIIPLSISLDDSIANKVLVYKSENQQKLNNTPLQYLRSGNSNGWSVDEFR | |
| | EVVIKMYNDKKISLKKLQNLLCEKDITKYDVRKGFIERNLVDTRYASRVVL | |
| | GLLKDYFKANNKNTKVFTVRGQITSQFRRAKNLKKDRNYFYHHIVDALIIA | |
| | HFRKFNYISELMQYGFNYKEKIDEETGELIQYNDTLFPKKDDDFTIQLRKL | |
| | NDLNIYSENIKISHKVDRKPNRQFSDETIYSARKIDNDYMKILKYKDIYGEE | |
| | GKKFAKKIKDNLQKENELDIFLMKKVDPETFNLLVSIVKNTICGEKENPFV | |
| | KYCEENNIDFIRKKSKNGNGPIIRSIRYYKKIESDYLDVSNKYINLPYNKKV | |
| | LLDSTKNFRIDFYKSKNGIYKFLVIKYYYIDKINNKYYIPLNIYEKLKKDRCIN | |
| | DSDEFLFSVYKNEYLRITTKDSKKNVPNIYRFIGTTHNTIELKPLYCDEEQE | |
| | KKRNRPTIGKKITKIEKFDVDVLGNKIAKKRDKCLQLEL | |
| AIYQ Type II | MIPNPLLGLDKRTADGSEFESPKKKRKVGSAKYILGLDMGITSVGWGLID | 39 |
| Cas | YDSGKIVDKGVRLFKEATAEDNLKRRTHRGSRRLKRRRQQRIIELKRLLK | |
| mammalian | KENILTDDYTPLNNVYSLRCKGLNEKLTNAELVSVLLHIAKIRGSSLEVVE | |
| expression | DDEKKANEDKSSKANLSVNETKLRTSKEEICEKQYKDLIENGKVRGINNIY | |
| construct | KSSSYESELKKILANQGLSKEVNDKIINIIFRRRDFNEGPGSMESPTIYGRF | |
| (includes N- | IPHKNGEVEVIHSMIEKMQGKCSIYQNEPRAAKMSFTADLFNFLNDLNNL | |
| terminal SV5 | EIQRADNYDNYITPDEKKLIINEYIIKKGKITIQKLCDVLQVKKENITGFRVN | |
| tag and NLS | KGEPILTEFKGYQKVLKALNRIIDTEIVDAVIDVLTMHKDVEKRKEEIKKINN | |
| and C- | TEFSEEDIVKLSEITGIQQYHSLSYKAMKVIIPELYENPENQMEVITRLNLV | |
| terminal NLS) | KTSIDDLKGRTTIPSYSDRIYSPVAKRVHNETIKVVNAVIKKYGDLDSIVIE | |
| (aa) | MARDRNTAEEQKNIEQVKKRNQENREKAKEIIEVNHSFSKLDSKLVQKIM | |
| | LYNQQDGKSMYSGKPINLNVLISDREAYEIDHIIPLSISLDDSIANKVLVYK | |
| | SENQQKLNNTPLQYLRSGNSNGWSVDEFREVVIKMYNDKKISLKKLQNL | |
| | LCEKDITKYDVRKGFIERNLVDTRYASRVVLGLLKDYFKANNKNTKVFTV | |
| | RGQITSQFRRAKNLKKDRNYFYHHIVDALIIAHFRKFNYISELMQYGFNYK | |
| | EKIDEETGELIQYNDTLFPKKDDDFTIQLRKLNDLNIYSENIKISHKVDRKP | |
| | NRQFSDETIYSARKIDNDYMKILKYKDIYGEEGKKFAKKIKDNLQKENELDI | |
| | FLMKKVDPETFNLLVSIVKNTICGEKENPFVKYCEENNIDFIRKKSKNGNG | |
| | PIIRSIRYYKKIESDYLDVSNKYINLPYNKKVLLDSTKNFRIDFYKSKNGIYK | |
| | FLVIKYYYIDKINNKYYIPLNIYEKLKKDRCINDSDEFLFSVYKNEYLRITTK | |
| | DSKKNVPNIYRFIGTTHNTIELKPLYCDEEQEKKRNRPTIGKKITKIEKFDV | |
| | DVLGNKIAKKRDKCLQLELSRKRTADGSEFESPKKKRKV | |
| AIYQ Type II | GTGGCAAAATATATATTAGGACTAGATATGGGAATTACTTCGGTAGGT | 40 |
| Cas coding | TGGGGCCTAATTGACTATGATTCTGGAAAAATAGTTGATAAAGGCGTT | |
| sequence (nt) | AGACTTTTTAAAGAAGCAACTGCGGAAGACAATTTAAAAAGACGTACA | |
| (not codon | CATCGTGGATCAAGAAGGTTAAAAAGACGTAGACAACAAAGAATAATT | |
| optimized) | GAATTAAAACGATTATTGAAAAAAGAAAACATCCTTACAGATGATTATA | |
| | CTCCTTTAAATAATGTTTACTCTCTTAGATGTAAAGGTTTAAATGAAAA | |
| | ATTAACAAATGCCGAACTTGTATCAGTATTATTACATATAGCAAAAATA | |
| | AGAGGAAGTTCCCTAGAAGTAGTTGAAGATGATGAAAAAAAGGCTAAT | |
| | GAGGATAAAAGTAGTAAAGCCAATTTATCAGTAAATGAAACAAAATTA | |
| | AGAACTTCAAAAGAAGAAATTTGTGAAAAACAATATAAAGATCTAATCG | |
| | AAAATGGTAAAGTTCGTGGAATAAATAATATTTATAAAAGTTCTTCATA | |
| | TGAGTCAGAACTCAAAAAAATTTTAGCTAATCAAGGCTTGTCAAAAGA | |
| | AGTTAATGATAAGATAATAAATATAATTTTCAGAAGACGTGACTTTAAT | |
| | GAAGGACCAGGAAGCATGGAATCGCCTACTATATATGGTAGATTTATA | |
| | CCACATAAAAATGGTGAGGTAGAAGTTATACATAGTATGATTGAAAAA | |
| | ATGCAAGGGAAATGTTCCATATACCAAAACGAACCTCGTGCAGCAAA | |
| | AATGTCATTTACAGCTGATTTGTTTAATTTTTTAAATGATTTAAACAATT | |
| | TAGAAATACAGCGTGCTGATAACTATGATAATTATATTACCCCAGACG | |
| | AAAAAAAGCTAATAATAAATGAGTATATCATAAAAAAAGGTAAAATTAC | |
| | AATTCAAAAATTATGCGATGTACTACAGGTAAAAAAAGAAAATATTACT | |
| | GGCTTTAGAGTTAACAAAGGTGAACCAATACTCACTGAATTTAAAGGT | |
| | TATCAAAAAGTATTAAAAGCTTTAAATAGAATAATTGATACTGAAATTG | |
| | TTGATGCGGTTATTGATGTTTTAACAATGCATAAAGATGTAGAAAAAA | |
| | GAAAAGAAGAAATTAAAAAAATAAACAATACAGAGTTTAGCGAAGAAG | |
| | ACATTGTAAAATTATCAGAAATAACTGGTATACAACAGTATCATTCATT | |
| | GTCTTATAAAGCAATGAAAGTAATAATACCTGAATTATATGAAAATCCT | |
| | GAAAATCAAATGGAAGTAATTACAAGATTAAATCTAGTGAAAACTTCAA | |
| | TAGATGATTTGAAAGGAAGAACAACAATACCTTCATATAGCGACAGAA | |
| | TATATTCCCCTGTAGCTAAACGTGTACATAATGAGACAATCAAAGTAG | |
| | TAAATGCGGTAATAAAAAAATATGGAGATTTAGATAGTATTGTTATAGA | |
| | AATGGCAAGGGATAGAAATACTGCTGAAGAACAAAAAAATATTGAACA | |
| | AGTCAAAAAGCGAAATCAAGAAAATAGAGAAAAAGCAAAAGAAATAAT | |
| | AGAAGTTAATCATAGTTTTTCAAAACTTGATTCAAAATTAGTTCAAAAG | |
| | ATTATGCTTTATAATCAACAAGATGGTAAAAGTATGTATAGTGGAAAAC | |
| | CAATAAACTTAAATGTTTTAATAAGTGACCGTGAAGCATACGAGATAG | |
| | ATCATATCATACCACTATCAATTTCACTTGATGATTCAATTGCCAATAA | |
| | AGTTTTGGTATATAAAAGTGAAAACCAACAAAAATTAAATAATACACCA | |
| | CTTCAATATTTACGTAGTGGTAACTCAAATGGCTGGAGCGTCGACGAA | |
| | TTCCGTGAAGTTGTAATAAAAATGTATAATGATAAAAAAATATCACTAA | |
| | AAAAATTACAAAACTTGTTATGTGAAAAAGATATAACAAAATATGATGT | |
| | AAGAAAAGGATTTATTGAAAGAAATTTAGTTGACACAAGATATGCAAG | |
| | TAGAGTAGTTTTGGGACTTCTAAAAGACTATTTCAAAGCTAACAATAAA | |
| | AATACTAAAGTATTTACAGTAAGAGGTCAAATCACATCACAGTTCAGA | |
| | AGAGCCAAAAACTTGAAAAAAGATAGAAATTACTTTTACCATCATATAG | |
| | TGGATGCATTAATAATTGCACATTTTAGGAAATTCAATTACATTAGTGA | |
| | ATTAATGCAATATGGCTTTAATTATAAAGAAAAAATCGATGAAGAAACA | |
| | GGAGAATTAATTCAATATAATGATACGTTGTTTCCAAAGAAGGATGAT | |
| | GATTTCACAATACAATTAAGAAAACTAAATGATCTTAATATATATAGTG | |
| | AAAACATCAAAATTTCGCATAAAGTTGATAGAAAGCCTAATAGACAGT | |
| | TTAGTGATGAAACAATCTATTCTGCGAGAAAAATCGACAACGATTATA | |
| | TGAAAATTTTGAAATATAAAGATATTTATGGTGAAGAAGGCAAAAAATT | |
| | TGCAAAAAAAATTAAAGATAATCTTCAAAAGGAAAATGAATTAGATATA | |
| | TTCTTAATGAAAAAAGTTGATCCAGAAACTTTCAATTTATTAGTTAGCA | |
| | TTGTTAAGAATACAATATGTGGTGAAAAAGAAAATCCATTTGTTAAATA | |
| | CTGTGAAGAAAACAATATAGATTTTATTAGAAAAAAATCTAAAAATGGC | |
| | AATGGTCCGATTATTAGATCTATTAGATATTATAAAAAAATAGAGAGTG | |
| | ACTATTTGGATGTTTCAAATAAATATATTAATTTACCATATAACAAAAAG | |
| | GTTTTGCTAGATAGCACTAAAAATTTTAGAATAGATTTTTATAAATCTAA | |
| | AAATGGCATTTATAAATTTTTGGTAATAAAGTATTACTATATAGATAAAA | |
| | TCAATAATAAATATTACATTCCATTAAATATATATGAAAAATTAAAAAAA | |
| | GATAGATGTATTAATGATAGTGATGAGTTCTTATTTTCAGTATATAAGA | |
| | ACGAGTATTTAAGAATTACAACCAAAGATAGTAAGAAAAATGTTCCAA | |
| | ATATTTATAGATTTATTGGAACAACACATAATACAATTGAATTAAAACC | |
| | GCTTTATTGTGACGAAGAACAAGAAAAAAAACGTAACAGACCAACCAT | |
| | AGGTAAAAAAATAACTAAAATTGAAAAGTTTGATGTTGATGTACTAGGT | |
| | AATAAAATTGCTAAAAAAAGAGATAAATGCTTGCAATTAGAGCTGTAG | |
| AIYQ Type II | GCTAAGTACATCCTGGGCCTGGATATGGGCATCACATCCGTGGGCTG | 41 |
| Cas coding | GGGCCTGATCGATTACGATAGCGGCAAAATCGTGGATAAGGGCGTG | |
| sequence (nt) | CGGCTGTTCAAGGAGGCCACCGCCGAGGATAACCTCAAGAGGAGGA | |
| (human | CCCATAGAGGAAGCAGACGGCTGAAGCGGCGGAGACAGCAAAGAAT | |
| codon- | CATCGAACTGAAGAGACTGCTGAAGAAGGAAAACATCCTGACAGACG | |
| optimized) | ATTATACACCTCTGAACAACGTCTACAGCCTGAGATGCAAGGGGCTG | |
| | AATGAAAAGCTCACAAACGCTGAGCTGGTGTCCGTGCTGCTGCACAT | |
| | CGCCAAAATCCGGGGAAGCAGCCTTGAAGTGGTGGAAGATGATGAG | |
| | AAGAAGGCTAATGAGGACAAGTCTAGCAAAGCAAACCTGAGCGTGAA | |
| | TGAAACAAAGCTCAGAACCAGCAAGGAAGAAATCTGCGAAAAACAGT | |
| | ACAAGGACCTGATTGAAAACGGCAAAGTTAGAGGCATCAACAACATC | |
| | TACAAGAGCTCCAGCTACGAAAGCGAGCTGAAGAAAATTCTCGCCAA | |
| | CCAGGGCCTGTCCAAGGAGGTGAACGACAAGATCATCAACATCATCT | |
| | TCAGACGGCGGGACTTCAACGAGGGCCCCGGCAGTATGGAATCTCC | |
| | AACAATCTACGGCCGATTTATCCCCCACAAGAACGGCGAGGTGGAAG | |
| | TGATCCATAGCATGATCGAAAAGATGCAGGGCAAGTGCAGCATCTAC | |
| | CAGAACGAACCTAGAGCCGCAAAGATGAGCTTCACCGCCGACCTCTT | |
| | CAACTTCCTGAACGACCTTAACAACCTGGAGATCCAGAGAGCCGATA | |
| | ATTATGACAACTACATCACACCTGATGAGAAGAAGCTGATCATAAACG | |
| | AGTACATCATCAAGAAGGGCAAGATTACGATTCAGAAGCTGTGCGAC | |
| | GTGCTGCAGGTAAAAAAGGAGAACATCACCGGCTTCCGCGTCAACAA | |
| | AGGAGAGCCCATCCTGACCGAGTTCAAGGGTTATCAGAAGGTGCTGA | |
| | AGGCTCTGAACAGAATCATCGACACCGAGATCGTGGATGCCGTGATC | |
| | GATGTGCTGACCATGCACAAGGATGTGGAAAAGCGGAAAGAAGAAAT | |
| | CAAGAAGATTAACAATACTGAGTTCAGCGAAGAGGACATCGTGAAAC | |
| | TGTCTGAGATCACAGGCATCCAGCAATACCACAGCCTGAGCTACAAG | |
| | GCCATGAAGGTCATCATCCCAGAGCTGTACGAGAATCCTGAAAACCA | |
| | GATGGAAGTGATCACCAGACTGAACCTGGTGAAAACCTCTATCGATG | |
| | ACCTGAAAGGAAGAACAACCATCCCCAGCTACAGCGACAGAATCTAC | |
| | AGCCCTGTTGCTAAAAGAGTGCACAATGAAACCATCAAGGTGGTTAAT | |
| | GCCGTGATTAAGAAGTACGGCGACCTGGATAGCATCGTTATCGAGAT | |
| | GGCCAGAGATAGGAACACAGCTGAAGAGCAGAAGAACATCGAACAG | |
| | GTGAAGAAGAGAAACCAGGAGAATAGAGAGAAGGCCAAGGAAATCAT | |
| | CGAAGTCAACCACAGTTTCAGCAAACTGGATTCTAAGCTGGTGCAGA | |
| | AAATCATGCTGTACAACCAGCAGGACGGCAAATCTATGTACTCTGGC | |
| | AAGCCCATCAACCTGAACGTGCTGATCAGCGACAGAGAGGCCTACGA | |
| | GATCGACCACATCATCCCTCTATCAATCTCTCTGGATGACAGCATCGC | |
| | CAATAAAGTGCTGGTCTACAAATCTGAGAACCAGCAAAAGCTGAATAA | |
| | CACCCCTCTGCAGTACCTGAGAAGCGGCAACAGCAACGGCTGGAGC | |
| | GTGGACGAGTTCAGAGAAGTGGTCATCAAGATGTACAACGACAAGAA | |
| | GATCTCCCTGAAGAAGCTGCAGAATCTCCTGTGTGAGAAGGACATAA | |
| | CCAAGTACGACGTGCGGAAGGGATTCATCGAGAGAAATCTGGTCGAC | |
| | ACCCGGTACGCCAGCAGAGTGGTGCTGGGACTGCTGAAGGACTACT | |
| | TTAAGGCCAACAATAAAAACACAAAAGTGTTCACAGTGCGCGGCCAG | |
| | ATCACCAGCCAGTTCAGACGGGCCAAGAACCTGAAAAAGGACAGAAA | |
| | CTACTTTTACCACCACATCGTGGACGCCCTGATCATCGCCCACTTCA | |
| | GAAAGTTTAACTACATCTCCGAGCTGATGCAATACGGATTTAACTACA | |
| | AGGAGAAGATTGACGAGGAAACCGGCGAGCTGATCCAGTACAATGA | |
| | CACACTGTTCCCTAAGAAAGACGATGATTTTACCATCCAGCTGCGGAA | |
| | GCTGAACGATCTGAACATCTACAGCGAAAACATCAAGATCAGCCACA | |
| | AAGTGGACAGAAAACCTAATAGACAATTCTCTGATGAAACCATCTATA | |
| | GCGCCCGGAAGATCGACAATGACTACATGAAAATCCTGAAGTACAAA | |
| | GATATCTACGGCGAGGAAGGCAAGAAGTTCGCCAAAAAGATCAAAGA | |
| | TAACCTGCAGAAGGAAAATGAGCTGGACATCTTCCTTATGAAGAAGG | |
| | TGGACCCTGAGACATTCAACCTGCTGGTTTCCATCGTGAAGAATACC | |
| | ATCTGTGGCGAAAAAGAGAACCCCTTCGTGAAATACTGCGAAGAGAA | |
| | CAACATCGACTTCATCAGAAAGAAAAGCAAGAACGGCAATGGCCCTA | |
| | TCATCCGGAGCATCAGATACTACAAAAAGATCGAGAGCGATTACCTG | |
| | GACGTGTCCAACAAGTACATCAATCTGCCTTACAACAAGAAGGTGCT | |
| | GCTCGATTCCACCAAGAACTTCCGGATCGACTTCTACAAGAGCAAGA | |
| | ATGGCATTTACAAGTTCCTGGTGATCAAGTATTATTACATCGACAAGA | |
| | TCAACAACAAATATTACATCCCACTGAACATCTATGAGAAGCTGAAGA | |
| | AAGACCGGTGCATCAACGACAGCGACGAGTTTCTGTTTAGCGTGTAC | |
| | AAAAACGAATACCTGCGGATCACAACAAAGGACAGCAAGAAAAACGT | |
| | GCCTAACATATACCGGTTCATCGGCACCACCCACAACACCATCGAGC | |
| | TGAAGCCTCTGTACTGTGACGAGGAACAGGAGAAGAAGCGGAACCG | |
| | CCCCACCATCGGCAAGAAGATTACCAAGATCGAGAAGTTCGACGTGG | |
| | ACGTCCTGGGAAACAAGATCGCCAAGAAGAGGGACAAGTGCCTGCA | |
| | GCTGGAACTG | |
| AIYQ Type II | ATGATCCCCAATCCCCTGCTGGGCCTGGACAAGCGGACTGCCGACG | 42 |
| Cas | GCAGTGAGTTCGAGTCTCCCAAGAAAAAGAGGAAAGTGggatccGCTAA | |
| mammalian | GTACATCCTGGGCCTGGATATGGGCATCACATCCGTGGGCTGGGGC | |
| expression | CTGATCGATTACGATAGCGGCAAAATCGTGGATAAGGGCGTGCGGCT | |
| construct | GTTCAAGGAGGCCACCGCCGAGGATAACCTCAAGAGGAGGACCCAT | |
| (includes N- | AGAGGAAGCAGACGGCTGAAGCGGCGGAGACAGCAAAGAATCATCG | |
| terminal SV5 | AACTGAAGAGACTGCTGAAGAAGGAAAACATCCTGACAGACGATTAT | |
| tag and NLS | ACACCTCTGAACAACGTCTACAGCCTGAGATGCAAGGGGCTGAATGA | |
| and C- | AAAGCTCACAAACGCTGAGCTGGTGTCCGTGCTGCTGCACATCGCCA | |
| terminal NLS) | AAATCCGGGGAAGCAGCCTTGAAGTGGTGGAAGATGATGAGAAGAA | |
| (nt) | GGCTAATGAGGACAAGTCTAGCAAAGCAAACCTGAGCGTGAATGAAA | |
| | CAAAGCTCAGAACCAGCAAGGAAGAAATCTGCGAAAAACAGTACAAG | |
| | GACCTGATTGAAAACGGCAAAGTTAGAGGCATCAACAACATCTACAA | |
| | GAGCTCCAGCTACGAAAGCGAGCTGAAGAAAATTCTCGCCAACCAGG | |
| | GCCTGTCCAAGGAGGTGAACGACAAGATCATCAACATCATCTTCAGA | |
| | CGGCGGGACTTCAACGAGGGCCCCGGCAGTATGGAATCTCCAACAA | |
| | TCTACGGCCGATTTATCCCCCACAAGAACGGCGAGGTGGAAGTGATC | |
| | CATAGCATGATCGAAAAGATGCAGGGCAAGTGCAGCATCTACCAGAA | |
| | CGAACCTAGAGCCGCAAAGATGAGCTTCACCGCCGACCTCTTCAACT | |
| | TCCTGAACGACCTTAACAACCTGGAGATCCAGAGAGCCGATAATTAT | |
| | GACAACTACATCACACCTGATGAGAAGAAGCTGATCATAAACGAGTA | |
| | CATCATCAAGAAGGGCAAGATTACGATTCAGAAGCTGTGCGACGTGC | |
| | TGCAGGTAAAAAAGGAGAACATCACCGGCTTCCGCGTCAACAAAGGA | |
| | GAGCCCATCCTGACCGAGTTCAAGGGTTATCAGAAGGTGCTGAAGGC | |
| | TCTGAACAGAATCATCGACACCGAGATCGTGGATGCCGTGATCGATG | |
| | TGCTGACCATGCACAAGGATGTGGAAAAGCGGAAAGAAGAAATCAAG | |
| | AAGATTAACAATACTGAGTTCAGCGAAGAGGACATCGTGAAACTGTCT | |
| | GAGATCACAGGCATCCAGCAATACCACAGCCTGAGCTACAAGGCCAT | |
| | GAAGGTCATCATCCCAGAGCTGTACGAGAATCCTGAAAACCAGATGG | |
| | AAGTGATCACCAGACTGAACCTGGTGAAAACCTCTATCGATGACCTG | |
| | AAAGGAAGAACAACCATCCCCAGCTACAGCGACAGAATCTACAGCCC | |
| | TGTTGCTAAAAGAGTGCACAATGAAACCATCAAGGTGGTTAATGCCGT | |
| | GATTAAGAAGTACGGCGACCTGGATAGCATCGTTATCGAGATGGCCA | |
| | GAGATAGGAACACAGCTGAAGAGCAGAAGAACATCGAACAGGTGAA | |
| | GAAGAGAAACCAGGAGAATAGAGAGAAGGCCAAGGAAATCATCGAA | |
| | GTCAACCACAGTTTCAGCAAACTGGATTCTAAGCTGGTGCAGAAAATC | |
| | ATGCTGTACAACCAGCAGGACGGCAAATCTATGTACTCTGGCAAGCC | |
| | CATCAACCTGAACGTGCTGATCAGCGACAGAGAGGCCTACGAGATCG | |
| | ACCACATCATCCCTCTATCAATCTCTCTGGATGACAGCATCGCCAATA | |
| | AAGTGCTGGTCTACAAATCTGAGAACCAGCAAAAGCTGAATAACACC | |
| | CCTCTGCAGTACCTGAGAAGCGGCAACAGCAACGGCTGGAGCGTGG | |
| | ACGAGTTCAGAGAAGTGGTCATCAAGATGTACAACGACAAGAAGATC | |
| | TCCCTGAAGAAGCTGCAGAATCTCCTGTGTGAGAAGGACATAACCAA | |
| | GTACGACGTGCGGAAGGGATTCATCGAGAGAAATCTGGTCGACACC | |
| | CGGTACGCCAGCAGAGTGGTGCTGGGACTGCTGAAGGACTACTTTAA | |
| | GGCCAACAATAAAAACACAAAAGTGTTCACAGTGCGCGGCCAGATCA | |
| | CCAGCCAGTTCAGACGGGCCAAGAACCTGAAAAAGGACAGAAACTAC | |
| | TTTTACCACCACATCGTGGACGCCCTGATCATCGCCCACTTCAGAAA | |
| | GTTTAACTACATCTCCGAGCTGATGCAATACGGATTTAACTACAAGGA | |
| | GAAGATTGACGAGGAAACCGGCGAGCTGATCCAGTACAATGACACAC | |
| | TGTTCCCTAAGAAAGACGATGATTTTACCATCCAGCTGCGGAAGCTG | |
| | AACGATCTGAACATCTACAGCGAAAACATCAAGATCAGCCACAAAGT | |
| | GGACAGAAAACCTAATAGACAATTCTCTGATGAAACCATCTATAGCGC | |
| | CCGGAAGATCGACAATGACTACATGAAAATCCTGAAGTACAAAGATAT | |
| | CTACGGCGAGGAAGGCAAGAAGTTCGCCAAAAAGATCAAAGATAACC | |
| | TGCAGAAGGAAAATGAGCTGGACATCTTCCTTATGAAGAAGGTGGAC | |
| | CCTGAGACATTCAACCTGCTGGTTTCCATCGTGAAGAATACCATCTGT | |
| | GGCGAAAAAGAGAACCCCTTCGTGAAATACTGCGAAGAGAACAACAT | |
| | CGACTTCATCAGAAAGAAAAGCAAGAACGGCAATGGCCCTATCATCC | |
| | GGAGCATCAGATACTACAAAAAGATCGAGAGCGATTACCTGGACGTG | |
| | TCCAACAAGTACATCAATCTGCCTTACAACAAGAAGGTGCTGCTCGAT | |
| | TCCACCAAGAACTTCCGGATCGACTTCTACAAGAGCAAGAATGGCAT | |
| | TTACAAGTTCCTGGTGATCAAGTATTATTACATCGACAAGATCAACAA | |
| | CAAATATTACATCCCACTGAACATCTATGAGAAGCTGAAGAAAGACCG | |
| | GTGCATCAACGACAGCGACGAGTTTCTGTTTAGCGTGTACAAAAACG | |
| | AATACCTGCGGATCACAACAAAGGACAGCAAGAAAAACGTGCCTAAC | |
| | ATATACCGGTTCATCGGCACCACCCACAACACCATCGAGCTGAAGCC | |
| | TCTGTACTGTGACGAGGAACAGGAGAAGAAGCGGAACCGCCCCACC | |
| | ATCGGCAAGAAGATTACCAAGATCGAGAAGTTCGACGTGGACGTCCT | |
| | GGGAAACAAGATCGCCAAGAAGAGGGACAAGTGCCTGCAGCTGGAA | |
| | CTGtctagaAAGCGGACAGCAGACGGCTCCGAATTTGAAAGCCCTAAGA | |
| | AAAAGAGAAAGGTGTGA | |

In some embodiments an AIYQ Type II Cas protein comprises an amino acid sequence of SEQ ID NO:37, SEQ ID NO:38 or SEQ ID NO:39. In some embodiments, an AIYQ Type II Cas protein has nickase activity, for example resulting from one or more amino acid substitutions relative to the sequence of SEQ ID NO:37, SEQ ID NO:38 or SEQ ID NO:39. In some embodiments, the one or more amino acid substitutions providing nickase activity is a D9A Substitution, wherein the position of the D9A substitution is defined with respect to the amino acid numbering of SEQ ID NO:38. In some embodiments, the one or more amino acid substitutions providing nickase activity is a N590A Substitution, wherein the position of the N590A substitution is defined with respect to the amino acid numbering of SEQ ID NO:38. In some embodiments, an AIYQ Type II Cas protein is catalytically inactive, for example due to both a D9A substitution and a N590A substitution, where the positions of the substitutions are defined with respect to the amino acid numbering of SEQ ID NO:38.

### 6.2.2. Type IIC Cas Proteins

### 6.2.2.1. EQSC Type II Cas Proteins

In one aspect, the disclosure provides EQSC Type II Cas proteins. EQSC Type II Cas proteins can be further classified as Type IIC Cas proteins. The EQSC Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:43. In some embodiments, the EQSC Type II Cas proteins comprise an amino acid sequence that is at least 95%, at least 96%, at least 97%, at least 98%, or at least 99% identical to SEQ ID NO:43. In some embodiments, a EQSC Type II Cas protein comprises an amino acid sequence that is identical to SEQ ID NO:43.

Exemplary EQSC Type II Cas protein sequences and nucleotide sequences encoding exemplary EQSC Type II Cas proteins are set forth in Table 2A.

TABLE 2A
EQSC Type II Cas Sequences

| | | SEQ ID |
|---|---|---|
| Name | Sequence | NO |
| EQSC Type II | VEVKTEAVGRLGRPRYLVLGMDPGIASCGFALLDINNHEILEMGVRLFDA | 43 |
| Cas amino | PLHPKTKQSLAAIRRGFRSTRRNIDRTQDRLKHCFECFKKYGVVPDDAD | |
| acid | KEYLHTVKGDKQPLKLRVDGLNRMLTNREWAMALYSLCKRRGYIPHGE | |
| sequence | GAQDKSSDDGKVLAAISANEKEIAAGKFRTAGEWLASRPTSRNRGGNYE | |
| (without N- | MCVKHEQLIEETRILFEAQRSFGSKVASEDFEREYIDICNWERPRDDFDR | |
| terminal | RTYGLVGSCVYFPQEKRAARCTLTSELVSAYGALGNITIVLGDGGVRRLT | |
| methionine) | AAERDECIRILFTADPNAKTRVVKFGALRKKLDLSSRDYFKGVPADDEKT | |
| | REVFKPSGWRALYSTLRESNPQLLARLRDDIDLADAVMEAAAYASSASS | |
| | LENCLSQLPLSDDEKQALCALPYSSKVLNGYGSRSKKALDLLLACLKEPE | |
| | VLNLTDAEAACGLDALRLGGPQLERSEKLIPYEAWVSAAGRTNNNPVVI | |
| | RAMSQMRKVVNAICGKWGVPNEIHVELDRELRLPKRAKEEIAKANKKNE | |
| | KERERIAVQAADILSCSPDEVSGKQIEKYRLWEEQENSDLYTGERIHVQR | |
| | LLSDDTYTQIDHILPFSRTGENSRNNKVLVLSESNQQKREQTPYEWMNS | |
| | GQPGAPSWDEFKLRVQANQHLKHRKKEFLLEQDLESKEVEFLQRNVTD | |
| | TAYMSREVCAYLSDCLLFPDDGMKVHVVPTTGRATAWLRRRWGLNFGA | |
| | TGEKDRSDDRHHATDACVIAACSRGLVIKTAKVMKGTHWSITRNMTSEE | |
| | RRDARMEALRESMPWESFADDVRARRDFVVPTRYVPRKGSGELFEQT | |
| | VYSYVGRNEKGQDLGRKKGVDKDKVMGNAVVSDDGKSVIKVSEMLCLR | |
| | LWHDPEIKKKGKVVGGWYADPVYKSDIPALKDGTYVPRIAKAHCGRSAW | |
| | APVPERLLLQRPVELYLGDVVRVKEMVGCLSGFDMGGVAWKFVDPMTR | |
| | ESISAPTIGSLTGKDAPKVVRGGIVG | |
| EQSC Type II | MVEVKTEAVGRLGRPRYLVLGMDPGIASCGFALLDINNHEILEMGVRLFD | 44 |
| Cas amino | APLHPKTKQSLAAIRRGFRSTRRNIDRTQDRLKHCFECFKKYGVVPDDA | |
| acid | DKEYLHTVKGDKQPLKLRVDGLNRMLTNREWAMALYSLCKRRGYIPHG | |
| sequence | EGAQDKSSDDGKVLAAISANEKEIAAGKFRTAGEWLASRPTSRNRGGNY | |
| | EMCVKHEQLIEETRILFEAQRSFGSKVASEDFEREYIDICNWERPRDDFD | |
| | RRTYGLVGSCVYFPQEKRAARCTLTSELVSAYGALGNITIVLGDGGVRRL | |
| | TAAERDECIRILFTADPNAKTRVVKFGALRKKLDLSSRDYFKGVPADDEK | |
| | TREVFKPSGWRALYSTLRESNPQLLARLRDDIDLADAVMEAAAYASSAS | |
| | SLENCLSQLPLSDDEKQALCALPYSSKVLNGYGSRSKKALDLLLACLKEP | |
| | EVLNLTDAEAACGLDALRLGGPQLERSEKLIPYEAWVSAAGRTNNNPVVI | |
| | RAMSQMRKVVNAICGKWGVPNEIHVELDRELRLPKRAKEEIAKANKKNE | |
| | KERERIAVQAADILSCSPDEVSGKQIEKYRLWEEQENSDLYTGERIHVQR | |
| | LLSDDTYTQIDHILPFSRTGENSRNNKVLVLSESNQQKREQTPYEWMNS | |
| | GQPGAPSWDEFKLRVQANQHLKHRKKEFLLEQDLESKEVEFLQRNVTD | |
| | TAYMSREVCAYLSDCLLFPDDGMKVHVVPTTGRATAWLRRRWGLNFGA | |
| | TGEKDRSDDRHHATDACVIAACSRGLVIKTAKVMKGTHWSITRNMTSEE | |
| | RRDARMEALRESMPWESFADDVRARRDFVVPTRYVPRKGSGELFEQT | |
| | VYSYVGRNEKGQDLGRKKGVDKDKVMGNAVVSDDGKSVIKVSEMLCLR | |
| | LWHDPEIKKKGKVVGGWYADPVYKSDIPALKDGTYVPRIAKAHCGRSAW | |
| | APVPERLLLQRPVELYLGDVVRVKEMVGCLSGFDMGGVAWKFVDPMTR | |
| | ESISAPTIGSLTGKDAPKVVRGGIVG | |
| EQSC Type II | MIPNPLLGLDKRTADGSEFESPKKKRKVGSVEVKTEAVGRLGRPRYLVL | 45 |
| Cas | GMDPGIASCGFALLDINNHEILEMGVRLFDAPLHPKTKQSLAAIRRGFRS | |
| mammalian | TRRNIDRTQDRLKHCFECFKKYGVVPDDADKEYLHTVKGDKQPLKLRVD | |
| expression | GLNRMLTNREWAMALYSLCKRRGYIPHGEGAQDKSSDDGKVLAAISAN | |
| construct | EKEIAAGKFRTAGEWLASRPTSRNRGGNYEMCVKHEQLIEETRILFEAQ | |
| (includes N- | RSFGSKVASEDFEREYIDICNWERPRDDFDRRTYGLVGSCVYFPQEKRA | |
| terminal SV5 | ARCTLTSELVSAYGALGNITIVLGDGGVRRLTAAERDECIRILFTADPNAK | |
| tag and NLS | TRVVKFGALRKKLDLSSRDYFKGVPADDEKTREVFKPSGWRALYSTLRE | |
| and C- | SNPQLLARLRDDIDLADAVMEAAAYASSASSLENCLSQLPLSDDEKQAL | |
| terminal NLS) | CALPYSSKVLNGYGSRSKKALDLLLACLKEPEVLNLTDAEAACGLDALRL | |
| (aa) | GGPQLERSEKLIPYEAWVSAAGRTNNNPVVIRAMSQMRKVVNAICGKW | |
| | GVPNEIHVELDRELRLPKRAKEEIAKANKKNEKERERIAVQAADILSCSPD | |
| | EVSGKQIEKYRLWEEQENSDLYTGERIHVQRLLSDDTYTQIDHILPFSRT | |
| | GENSRNNKVLVLSESNQQKREQTPYEWMNSGQPGAPSWDEFKLRVQA | |
| | NQHLKHRKKEFLLEQDLESKEVEFLQRNVTDTAYMSREVCAYLSDCLLF | |
| | PDDGMKVHVVPTTGRATAWLRRRWGLNFGATGEKDRSDDRHHATDAC | |
| | VIAACSRGLVIKTAKVMKGTHWSITRNMTSEERRDARMEALRESMPWES | |
| | FADDVRARRDFVVPTRYVPRKGSGELFEQTVYSYVGRNEKGQDLGRKK | |
| | GVDKDKVMGNAVVSDDGKSVIKVSEMLCLRLWHDPEIKKKGKVVGGWY | |
| | ADPVYKSDIPALKDGTYVPRIAKAHCGRSAWAPVPERLLLQRPVELYLG | |
| | DWVRVKEMVGCLSGFDMGGVAWKFVDPMTRESISAPTIGSLTGKDAPK | |
| | VVRGGIVGSRKRTADGSEFESPKKKRKV | |
| EQSC Type II | ATGGTCGAGGTTAAAACAGAGGCGGTCGGTCGGCTCGGTCGTCCTC | 46 |
| Cas coding | GATATCTGGTTTTGGGAATGGACCCAGGCATTGCGAGTTGCGGCTTC | |
| sequence (nt) | GCGTTGCTCGATATCAACAATCATGAGATCTTGGAAATGGGCGTTCG | |
| (not codon | CTTGTTCGATGCCCCGTTGCATCCTAAAACGAAGCAGAGCTTGGCCG | |
| optimized) | CTATTCGTCGCGGCTTCCGTTCGACGCGGCGCAATATCGACCGAACG | |
| | CAGGATCGACTCAAGCATTGCTTCGAGTGCTTTAAGAAATACGGCGT | |
| | CGTTCCAGATGATGCCGATAAAGAATATCTTCATACGGTCAAAGGGG | |
| | ATAAACAGCCCCTCAAGCTTCGTGTCGATGGCCTGAATCGCATGCTC | |
| | ACGAACAGGGAGTGGGCCATGGCCCTCTATTCTCTGTGCAAGCGTC | |
| | GTGGCTACATTCCGCACGGCGAAGGCGCTCAGGATAAATCGAGCGA | |
| | TGACGGCAAGGTGCTTGCTGCGATCAGCGCGAATGAAAAAGAAATCG | |
| | CAGCAGGAAAATTCAGAACCGCAGGCGAATGGCTCGCGTCGCGTCC | |
| | CACGAGCAGGAATCGCGGGGGCAATTACGAGATGTGCGTCAAGCAC | |
| | GAGCAGCTTATCGAAGAAACCCGTATTCTGTTCGAAGCACAGCGCTC | |
| | GTTCGGTTCCAAGGTTGCCTCTGAAGATTTCGAGCGCGAATACATCG | |
| | ACATATGCAATTGGGAACGGCCGCGCGACGATTTCGATAGGCGTACG | |
| | TACGGCCTGGTGGGGTCATGCGTTTACTTTCCGCAAGAGAAGCGCG | |
| | CGGCACGTTGCACGCTTACGAGCGAGCTTGTTTCCGCGTATGGCGCT | |
| | TTGGGAAATATAACCATCGTGCTCGGCGACGGCGGCGTGCGCCGTC | |
| | TTACGGCGGCCGAACGAGACGAATGCATTCGCATATTGTTTACGGCC | |
| | GATCCGAATGCGAAGACCCGCGTTGTGAAGTTCGGTGCCTTGCGCAA | |
| | AAAGCTCGACTTGAGTTCGCGCGATTATTTCAAGGGCGTTCCTGCCG | |
| | ATGACGAAAAGACCCGTGAGGTGTTCAAACCCAGCGGTTGGCGCGC | |
| | CTTGTATTCGACGTTGCGGGAATCCAATCCGCAGCTTCTCGCAAGGT | |
| | TGAGGGACGATATCGATTTGGCGGATGCCGTTATGGAGGCCGCCGC | |
| | GTATGCATCATCTGCGTCGAGCTTGGAGAACTGCCTCAGCCAGCTTC | |
| | CTCTTAGCGATGATGAAAAGCAGGCGCTTTGCGCGCTGCCGTATTCG | |
| | TCTAAAGTGCTGAATGGATATGGAAGCCGCTCGAAAAAGGCGCTCGA | |
| | CTTGCTGCTTGCTTGTCTCAAAGAACCCGAAGTGCTCAATCTGACCG | |
| | ATGCCGAGGCGGCATGTGGGTTGGATGCGTTGCGTCTTGGCGGCCC | |
| | ACAGCTCGAGCGTTCCGAGAAGCTGATTCCCTATGAGGCCTGGGTAA | |
| | GCGCGGCGGGTCGTACGAATAACAATCCTGTCGTCATTCGTGCTATG | |
| | TCGCAAATGCGCAAGGTCGTAAATGCCATATGCGGCAAATGGGGCGT | |
| | TCCGAACGAAATCCACGTCGAACTTGACCGCGAGCTTCGGTTGCCTA | |
| | AGCGTGCGAAAGAAGAAATTGCCAAGGCCAATAAGAAAAACGAGAAA | |
| | GAGCGCGAGCGCATAGCCGTTCAGGCCGCGGACATTCTTTCCTGCA | |
| | GCCCCGATGAGGTGAGCGGAAAGCAGATAGAGAAGTACCGCTTGTG | |
| | GGAGGAGCAGGAAAACTCCGATTTGTATACGGGCGAGCGTATCCAC | |
| | GTCCAACGCCTTTTGAGCGATGACACCTATACGCAGATCGACCATATT | |
| | CTTCCGTTTTCGCGAACGGGAGAGAATTCGCGTAATAACAAGGTGCT | |
| | CGTGCTTTCGGAAAGCAATCAGCAGAAACGCGAGCAGACTCCGTATG | |
| | AATGGATGAATTCGGGCCAGCCTGGTGCTCCGTCGTGGGACGAGTT | |
| | CAAACTTCGAGTTCAGGCGAACCAGCATCTTAAGCACCGCAAGAAAG | |
| | AATTCTTGCTCGAGCAGGATTTGGAATCGAAGGAAGTCGAATTCCTC | |
| | CAGCGAAACGTGACCGACACGGCTTATATGTCGCGCGAAGTCTGCG | |
| | CCTATCTGTCCGATTGCCTGTTGTTCCCCGATGACGGCATGAAGGTT | |
| | CACGTTGTTCCCACGACGGGTCGCGCTACGGCATGGTTGCGTCGCC | |
| | GATGGGGGCTGAACTTCGGAGCAACGGGGGAGAAGGACCGTTCGGA | |
| | CGACCGTCATCATGCTACGGACGCCTGCGTTATTGCCGCGTGCAGCA | |
| | GGGGCCTTGTCATCAAGACGGCAAAGGTTATGAAGGGAACGCACTG | |
| | GAGCATCACGCGTAATATGACGTCGGAGGAGCGCAGAGATGCCCGT | |
| | ATGGAGGCGTTGCGTGAGAGCATGCCCTGGGAAAGCTTCGCCGACG | |
| | ATGTCCGCGCACGTCGCGATTTCGTGGTACCCACGCGCTATGTGCCT | |
| | CGTAAAGGCAGCGGTGAGCTTTTCGAGCAGACAGTGTATTCGTATGT | |
| | CGGTCGGAACGAAAAAGGGCAGGACCTTGGCCGAAAGAAGGGCGTC | |
| | GACAAAGATAAGGTCATGGGCAATGCCGTAGTGTCCGATGATGGAAA | |
| | ATCGGTCATTAAGGTGAGCGAGATGCTGTGCCTGCGTTTGTGGCACG | |
| | ATCCAGAGATTAAAAAGAAGGGGAAGGTCGTTGGCGGCTGGTATGCC | |
| | GACCCGGTGTACAAGTCTGATATTCCCGCTCTTAAGGATGGCACGTA | |
| | TGTGCCGCGAATCGCAAAGGCCCACTGCGGCCGTTCGGCGTGGGCG | |
| | CCTGTTCCCGAACGGCTTTTGCTCCAGCGTCCGGTTGAGTTGTATCT | |
| | TGGTGATGTTGTCAGGGTGAAAGAGATGGTTGGGTGCCTTTCTGGTT | |
| | TTGACATGGGTGGTGTTGCATGGAAGTTCGTTGACCCAATGACGCGT | |
| | GAGTCGATATCTGCGCCGACCATTGGAAGTCTTACGGGCAAGGATGC | |
| | TCCCAAGGTGGTTCGTGGGGGCATTGTCGGTTAG | |
| EQSC Type II | GTGGAAGTGAAAACCGAAGCCGTGGGCAGACTGGGCCGGCCTAGAT | 47 |
| Cas coding | ATCTGGTCCTGGGCATGGACCCCGGCATCGCCTCCTGCGGCTTTGC | |
| sequence (nt) | CCTCCTGGACATCAACAATCACGAGATCCTGGAAATGGGAGTTCGGC | |
| (human | TGTTTGACGCACCTCTGCACCCTAAAACTAAGCAGAGCCTGGCCGCC | |
| codon- | ATCCGGAGGGGATTTCGGTCCACCCGGAGAAACATCGACCGGACCC | |
| optimized) | AGGACAGGCTCAAACATTGTTTTGAGTGCTTCAAGAAATACGGCGTG | |
| | GTTCCTGACGATGCCGACAAGGAGTACCTGCACACCGTGAAAGGAG | |
| | ATAAGCAACCTCTGAAGCTGAGAGTGGACGGTCTGAACAGAATGCTG | |
| | ACCAATAGAGAGTGGGCTATGGCCCTGTATTCTCTGTGCAAGCGGAG | |
| | AGGGTACATCCCCCACGGCGAGGGAGCTCAGGACAAAAGCAGCGAC | |
| | GATGGAAAAGTGCTCGCTGCAATTAGCGCTAACGAGAAGGAGATTGC | |
| | CGCCGGAAAGTTCAGAACCGCCGGCGAATGGCTGGCCAGCCGGCC | |
| | CACCTCCAGAAATAGAGGCGGCAACTACGAAATGTGCGTGAAGCACG | |
| | AGCAATTGATCGAGGAAACCAGAATCCTGTTCGAGGCCCAGCGGAG | |
| | CTTCGGCAGTAAGGTCGCTTCTGAGGACTTCGAGCGGGAGTACATCG | |
| | ACATCTGCAACTGGGAACGGCCTCGGGACGACTTCGACCGGCGCAC | |
| | CTACGGCCTGGTGGGGAGCTGCGTGTACTTCCCCCAGGAGAAAAGA | |
| | GCCGCTCGGTGCACCCTGACATCCGAGCTGGTGTCTGCCTATGGAG | |
| | CACTGGGCAATATCACAATCGTGCTGGGCGATGGCGGCGTGCGCCG | |
| | GCTGACCGCCGCCGAACGGGACGAGTGCATCAGAATCCTGTTCACC | |
| | GCCGATCCTAACGCCAAGACCAGAGTGGTGAAGTTCGGCGCCTTAC | |
| | GAAAAAAGCTGGACCTGTCCAGCAGAGACTACTTCAAGGGCGTGCCC | |
| | GCCGATGATGAAAAGACCAGAGAAGTCTTTAAGCCAAGCGGCTGGC | |
| | GCGCCCTGTACAGTACACTGAGAGAGAGCAACCCCCAGCTGCTGGC | |
| | CCGATTACGAGATGATATCGATCTCGCCGATGCTGTGATGGAAGCCG | |
| | CTGCTTACGCCAGCAGCGCCAGCTCTCTGGAAAACTGCCTGAGCCA | |
| | GCTGCCTCTGAGCGACGACGAGAAGCAGGCCTTGTGCGCCCTGCCA | |
| | TACTCAAGCAAAGTGCTGAACGGCTACGGCAGCAGATCTAAGAAGGC | |
| | CCTGGACCTTCTGCTGGCCTGTCTCAAGGAGCCTGAAGTTCTCAACC | |
| | TGACCGATGCTGAAGCTGCTTGTGGCCTCGATGCCCTGAGACTGGG | |
| | CGGTCCTCAGCTGGAACGGAGTGAAAAGCTGATACCCTACGAGGCC | |
| | TGGGTGAGCGCTGCCGGCCGTACCAACAACAATCCCGTGGTGATTA | |
| | GAGCCATGAGCCAAATGAGAAAGGTGGTCAACGCCATCTGCGGCAA | |
| | GTGGGGCGTGCCAAATGAGATCCACGTGGAACTGGATCGGGAACTG | |
| | CGGCTGCCCAAGCGGGCTAAAGAAGAGATCGCCAAGGCCAACAAGA | |
| | AGAACGAGAAAGAGAGAGAGAGAATCGCCGTGCAAGCCGCCGACAT | |
| | ACTGAGTTGTTCTCCTGATGAGGTGTCTGGCAAGCAGATCGAGAAGT | |
| | ACAGACTGTGGGAGGAACAGGAGAATAGCGACCTGTACACAGGCGA | |
| | GAGAATCCACGTCCAGAGACTTCTGAGCGACGATACCTATACACAGA | |
| | TCGACCACATCCTGCCCTTCAGCCGCACAGGCGAGAACAGCAGAAA | |
| | CAACAAGGTGCTCGTGCTGTCCGAAAGCAACCAGCAGAAGAGAGAG | |
| | CAGACCCCTTATGAGTGGATGAACAGCGGACAGCCTGGCGCTCCCA | |
| | GCTGGGACGAGTTCAAGCTGCGGGTGCAGGCCAACCAGCACCTGAA | |
| | GCATAGGAAGAAAGAGTTCCTGCTGGAGCAGGATCTGGAATCTAAGG | |
| | AAGTGGAGTTCCTGCAGAGAAACGTGACCGACACCGCCTACATGAGC | |
| | CGGGAAGTGTGCGCCTACCTGAGCGACTGCCTGCTCTTCCCTGACG | |
| | ACGGCATGAAGGTGCACGTGGTGCCCACAACAGGCAGAGCCACAGC | |
| | CTGGCTGAGAAGAAGATGGGGACTGAACTTCGGCGCTACCGGCGAG | |
| | AAAGACAGAAGCGATGACCGGCACCACGCCACCGACGCTTGCGTGA | |
| | TCGCCGCCTGTAGCAGAGGCCTGGTGATCAAGACAGCCAAGGTCAT | |
| | GAAGGGCACCCACTGGAGCATCACCAGAAACATGACAAGCGAGGAA | |
| | AGAAGAGATGCCCGGATGGAAGCTCTGAGAGAGAGCATGCCTTGGG | |
| | AGTCCTTCGCCGACGACGTGAGAGCTAGAAGAGATTTTGTGGTGCCA | |
| | ACACGGTACGTGCCTAGAAAGGGCTCTGGAGAGCTGTTCGAGCAGA | |
| | CCGTCTACAGCTACGTGGGAAGAAATGAAAAGGGCCAGGACCTGGG | |
| | ACGGAAGAAAGGCGTTGACAAGGACAAGGTGATGGGCAACGCCGTG | |
| | GTGAGCGACGACGGCAAGTCCGTGATCAAGGTGAGCGAAATGCTGT | |
| | GCCTGAGACTGTGGCACGACCCTGAGATCAAGAAGAAAGGCAAAGTT | |
| | GTCGGCGGCTGGTACGCCGACCCTGTGTACAAGAGCGACATCCCTG | |
| | CCCTGAAGGATGGCACATACGTGCCTAGAATCGCCAAGGCCCACTG | |
| | CGGCAGATCCGCGTGGGCCCCAGTACCAGAGAGACTGCTGCTGCAG | |
| | CGGCCTGTGGAACTGTACCTGGGCGATGTGGTGCGGGTGAAGGAGA | |
| | TGGTGGGTTGTCTGAGCGGCTTCGACATGGGCGGCGTGGCCTGGAA | |
| | ATTCGTGGACCCTATGACCAGAGAGAGCATCAGCGCCCCTACCATCG | |
| | GCTCCCTGACCGGCAAGGACGCCCCTAAGGTGGTGCGGGGCGGCA | |
| | TCGTGGGC | |
| EQSC Type II | ATGATCCCCAATCCCCTGCTGGGCCTGGACAAGCGGACTGCCGACG | 48 |
| Cas | GCAGTGAGTTCGAGTCTCCCAAGAAAAAGAGGAAAGTGggatccGTGG | |
| mammalian | AAGTGAAAACCGAAGCCGTGGGCAGACTGGGCCGGCCTAGATATCT | |
| expression | GGTCCTGGGCATGGACCCCGGCATCGCCTCCTGCGGCTTTGCCCTC | |
| construct | CTGGACATCAACAATCACGAGATCCTGGAAATGGGAGTTCGGCTGTT | |
| (includes N- | TGACGCACCTCTGCACCCTAAAACTAAGCAGAGCCTGGCCGCCATCC | |
| terminal SV5 | GGAGGGGATTTCGGTCCACCCGGAGAAACATCGACCGGACCCAGGA | |
| tag and NLS | CAGGCTCAAACATTGTTTTGAGTGCTTCAAGAAATACGGCGTGGTTCC | |
| and C- | TGACGATGCCGACAAGGAGTACCTGCACACCGTGAAAGGAGATAAG | |
| terminal NLS) | CAACCTCTGAAGCTGAGAGTGGACGGTCTGAACAGAATGCTGACCAA | |
| nt) | TAGAGAGTGGGCTATGGCCCTGTATTCTCTGTGCAAGCGGAGAGGGT | |
| | ACATCCCCCACGGCGAGGGAGCTCAGGACAAAAGCAGCGACGATGG | |
| | AAAAGTGCTCGCTGCAATTAGCGCTAACGAGAAGGAGATTGCCGCCG | |
| | GAAAGTTCAGAACCGCCGGCGAATGGCTGGCCAGCCGGCCCACCTC | |
| | CAGAAATAGAGGCGGCAACTACGAAATGTGCGTGAAGCACGAGCAAT | |
| | TGATCGAGGAAACCAGAATCCTGTTCGAGGCCCAGCGGAGCTTCGG | |
| | CAGTAAGGTCGCTTCTGAGGACTTCGAGCGGGAGTACATCGACATCT | |
| | GCAACTGGGAACGGCCTCGGGACGACTTCGACCGGCGCACCTACGG | |
| | CCTGGTGGGGAGCTGCGTGTACTTCCCCCAGGAGAAAAGAGCCGCT | |
| | CGGTGCACCCTGACATCCGAGCTGGTGTCTGCCTATGGAGCACTGG | |
| | GCAATATCACAATCGTGCTGGGCGATGGCGGCGTGCGCCGGCTGAC | |
| | CGCCGCCGAACGGGACGAGTGCATCAGAATCCTGTTCACCGCCGAT | |
| | CCTAACGCCAAGACCAGAGTGGTGAAGTTCGGCGCCTTACGAAAAAA | |
| | GCTGGACCTGTCCAGCAGAGACTACTTCAAGGGCGTGCCCGCCGAT | |
| | GATGAAAAGACCAGAGAAGTCTTTAAGCCAAGCGGCTGGCGCGCCC | |
| | TGTACAGTACACTGAGAGAGAGCAACCCCCAGCTGCTGGCCCGATTA | |
| | CGAGATGATATCGATCTCGCCGATGCTGTGATGGAAGCCGCTGCTTA | |
| | CGCCAGCAGCGCCAGCTCTCTGGAAAACTGCCTGAGCCAGCTGCCT | |
| | CTGAGCGACGACGAGAAGCAGGCCTTGTGCGCCCTGCCATACTCAA | |
| | GCAAAGTGCTGAACGGCTACGGCAGCAGATCTAAGAAGGCCCTGGA | |
| | CCTTCTGCTGGCCTGTCTCAAGGAGCCTGAAGTTCTCAACCTGACCG | |
| | ATGCTGAAGCTGCTTGTGGCCTCGATGCCCTGAGACTGGGCGGTCC | |
| | TCAGCTGGAACGGAGTGAAAAGCTGATACCCTACGAGGCCTGGGTG | |
| | AGCGCTGCCGGCCGTACCAACAACAATCCCGTGGTGATTAGAGCCAT | |
| | GAGCCAAATGAGAAAGGTGGTCAACGCCATCTGCGGCAAGTGGGGC | |
| | GTGCCAAATGAGATCCACGTGGAACTGGATCGGGAACTGCGGCTGC | |
| | CCAAGCGGGCTAAAGAAGAGATCGCCAAGGCCAACAAGAAGAACGA | |
| | GAAAGAGAGAGAGAGAATCGCCGTGCAAGCCGCCGACATACTGAGT | |
| | TGTTCTCCTGATGAGGTGTCTGGCAAGCAGATCGAGAAGTACAGACT | |
| | GTGGGAGGAACAGGAGAATAGCGACCTGTACACAGGCGAGAGAATC | |
| | CACGTCCAGAGACTTCTGAGCGACGATACCTATACACAGATCGACCA | |
| | CATCCTGCCCTTCAGCCGCACAGGCGAGAACAGCAGAAACAACAAG | |
| | GTGCTCGTGCTGTCCGAAAGCAACCAGCAGAAGAGAGAGCAGACCC | |
| | CTTATGAGTGGATGAACAGCGGACAGCCTGGCGCTCCCAGCTGGGA | |
| | CGAGTTCAAGCTGCGGGTGCAGGCCAACCAGCACCTGAAGCATAGG | |
| | AAGAAAGAGTTCCTGCTGGAGCAGGATCTGGAATCTAAGGAAGTGGA | |
| | GTTCCTGCAGAGAAACGTGACCGACACCGCCTACATGAGCCGGGAA | |
| | GTGTGCGCCTACCTGAGCGACTGCCTGCTCTTCCCTGACGACGGCAT | |
| | GAAGGTGCACGTGGTGCCCACAACAGGCAGAGCCACAGCCTGGCTG | |
| | AGAAGAAGATGGGGACTGAACTTCGGCGCTACCGGCGAGAAAGACA | |
| | GAAGCGATGACCGGCACCACGCCACCGACGCTTGCGTGATCGCCGC | |
| | CTGTAGCAGAGGCCTGGTGATCAAGACAGCCAAGGTCATGAAGGGC | |
| | ACCCACTGGAGCATCACCAGAAACATGACAAGCGAGGAAAGAAGAGA | |
| | TGCCCGGATGGAAGCTCTGAGAGAGAGCATGCCTTGGGAGTCCTTC | |
| | GCCGACGACGTGAGAGCTAGAAGAGATTTTGTGGTGCCAACACGGTA | |
| | CGTGCCTAGAAAGGGCTCTGGAGAGCTGTTCGAGCAGACCGTCTAC | |
| | AGCTACGTGGGAAGAAATGAAAAGGGCCAGGACCTGGGACGGAAGA | |
| | AAGGCGTTGACAAGGACAAGGTGATGGGCAACGCCGTGGTGAGCGA | |
| | CGACGGCAAGTCCGTGATCAAGGTGAGCGAAATGCTGTGCCTGAGA | |
| | CTGTGGCACGACCCTGAGATCAAGAAGAAAGGCAAAGTTGTCGGCG | |
| | GCTGGTACGCCGACCCTGTGTACAAGAGCGACATCCCTGCCCTGAA | |
| | GGATGGCACATACGTGCCTAGAATCGCCAAGGCCCACTGCGGCAGA | |
| | TCCGCGTGGGCCCCAGTACCAGAGAGACTGCTGCTGCAGCGGCCTG | |
| | TGGAACTGTACCTGGGCGATGTGGTGCGGGTGAAGGAGATGGTGGG | |
| | TTGTCTGAGCGGCTTCGACATGGGCGGCGTGGCCTGGAAATTCGTG | |
| | GACCCTATGACCAGAGAGAGCATCAGCGCCCCTACCATCGGCTCCCT | |
| | GACCGGCAAGGACGCCCCTAAGGTGGTGCGGGGCGGCATCGTGGG | |
| | CtctagaAAGCGGACAGCAGACGGCTCCGAATTTGAAAGCCCTAAGAAA | |
| | AAGAGAAAGGTGTGA | |

In some embodiments an EQSC Type II Cas protein comprises an amino acid sequence of SEQ ID NO:43, SEQ ID NO:44, or SEQ ID:45. In some embodiments, an EQSCType II Cas protein has nickase activity, for example resulting from one or more amino acid substitutions relative to the sequence of SEQ ID NO:43, SEQ ID NO:44, or SEQ ID:45. In some embodiments, the one or more amino acid substitutions providing nickase activity is a D23A Substitution, wherein the position of the D23A substitution is defined with respect to the amino acid numbering of SEQ ID NO:44. In some embodiments, the one or more amino acid substitutions providing nickase activity is a N629A Substitution, wherein the position of the N629A substitution is defined with respect to the amino acid numbering of SEQ ID NO:44. In some embodiments, an EQSC Type II Cas protein is catalytically inactive, for example due to both a D23A substitution and a N629A substitution, where the positions of the substitutions are defined with respect to the amino acid numbering of SEQ ID NO:44.

### 6.2.2.2. BDLP Type II Cas Proteins

In one aspect, the disclosure provides BDLP Type II Cas proteins. BDLP Type II Cas proteins can be further classified as Type IIC Cas proteins. The BDLP Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:49. In some embodiments, the BDLP Type II Cas proteins comprise an amino acid sequence that is at least 95%, at least 96%, at least 97%, at least 98%, or at least 99% identical to SEQ ID NO:49. In some embodiments, a BDLP Type II Cas protein comprises an amino acid sequence that is identical to SEQ ID NO:49.

Exemplary BDLP Type II Cas protein sequences and nucleotide sequences encoding exemplary BDLP Type II Cas proteins are set forth in Table 2B.

TABLE 2B
BDLP Type II Cas Sequences

| | | SEQ ID |
|---|---|---|
| Name | Sequence | NO |
| BDLP Type II | VEVKTEAVGRLGRPRYLVLGMDPGIASCGFALLDVNNHEILEMGVRLFD | 49 |
| Cas amino | APLHPKTKQSLAAIRRGFRSTRRNIDRTQDRLKHCLKCFKKYGVVPDDA | |
| acid | DKEYLHMVKGDKRPLELRVDGLNRMLTNREWGMVLYSLCKRRGYIPHG | |
| sequence | EGAQDKSSDDGKVLAAISANEKEIAAGEFKTVGEWLASRPASRNHGGNY | |
| (without N- | KMCVKHEQLIEETRILFEAQRSFGSKVASEDFEREYIDICNWERPRDDFD | |
| terminal | RRTYGLVGSCVYFPQEKRAARCTLTSELVSAYGALGNITIVLGDGGTRRL | |
| methionine) | TAAERNECICVLFTADPNAKTRVVKFGALRKKLDLGSRDYFKGVPADDE | |
| | KTREVFKPSGWRALYSTLRESNPQLLARLKNDIDLADAVMEAAAYASSA | |
| | SSLDNCLSQLPLDDDEKQALCALPYSSKVLNGYGSRSKKALDLLLACLEE | |
| | PEVLNLTDAEAACGLDAMRLGGPQLERSEKLIPYEAWVSATGRTNNNPV | |
| | VIRAMSQMRKVVNAICGKWGVPNEIHVELDRELRLPKRAKEEIAKANRKN | |
| | EKERERIAIQAADILSCSPDEVSGKQIEKYRLWEEQENSDLYTGERIHVQ | |
| | RLLNDDTYTQIDHILPFSRTGENSRNNKVLVLSESNQQKREQTPYEWMN | |
| | SGHPGAPSWDEFKLRVQANQHLKHRKKEFLLEQDLESKEVEFLQRNVT | |
| | DTAYMSREVCAYLSDCLLFPDDGMKVHVVPTTGRATAWLRRRWGLNF | |
| | GATGEKDRSDDRHHATDACVIAACSRSLVIKTAKVMKGTHWSVTRHMT | |
| | PEERRDARMEALRESMPWESFANDVRDRRDSVVPTRYVPRKGSGELF | |
| | EQTVYSYAGEVDSLSRQVIGKSGGKASPKANVFILDDGKSALLVGDMLC | |
| | LRLWFDPEVKKKGKVVGGWYANPLYKADLPDLRKGTYVPKAVKPRHGR | |
| | SAWLEIPERMLEQKPVEIYRGDLVEIDGKNGCFVGYNIANGSWTFVDPLT | |
| | REEIDMPSLASLSSQTNLKVVRRGLLPMD | |
| BDLP Type II | MVEVKTEAVGRLGRPRYLVLGMDPGIASCGFALLDVNNHEILEMGVRLF | 50 |
| Cas amino | DAPLHPKTKQSLAAIRRGFRSTRRNIDRTQDRLKHCLKCFKKYGVVPDD | |
| acid | ADKEYLHMVKGDKRPLELRVDGLNRMLTNREWGMVLYSLCKRRGYIPH | |
| sequence | GEGAQDKSSDDGKVLAAISANEKEIAAGEFKTVGEWLASRPASRNHGG | |
| | NYKMCVKHEQLIEETRILFEAQRSFGSKVASEDFEREYIDICNWERPRDD | |
| | FDRRTYGLVGSCVYFPQEKRAARCTLTSELVSAYGALGNITIVLGDGGTR | |
| | RLTAAERNECICVLFTADPNAKTRVVKFGALRKKLDLGSRDYFKGVPAD | |
| | DEKTREVFKPSGWRALYSTLRESNPQLLARLKNDIDLADAVMEAAAYAS | |
| | SASSLDNCLSQLPLDDDEKQALCALPYSSKVLNGYGSRSKKALDLLLACL | |
| | EEPEVLNLTDAEAACGLDAMRLGGPQLERSEKLIPYEAWVSATGRTNNN | |
| | PVVIRAMSQMRKVVNAICGKWGVPNEIHVELDRELRLPKRAKEEIAKANR | |
| | KNEKERERIAIQAADILSCSPDEVSGKQIEKYRLWEEQENSDLYTGERIH | |
| | VQRLLNDDTYTQIDHILPFSRTGENSRNNKVLVLSESNQQKREQTPYEW | |
| | MNSGHPGAPSWDEFKLRVQANQHLKHRKKEFLLEQDLESKEVEFLQRN | |
| | VTDTAYMSREVCAYLSDCLLFPDDGMKVHVVPTTGRATAWLRRRWGLN | |
| | FGATGEKDRSDDRHHATDACVIAACSRSLVIKTAKVMKGTHWSVTRHMT | |
| | PEERRDARMEALRESMPWESFANDVRDRRDSVVPTRYVPRKGSGELF | |
| | EQTVYSYAGEVDSLSRQVIGKSGGKASPKANVFILDDGKSALLVGDMLC | |
| | LRLWFDPEVKKKGKVVGGWYANPLYKADLPDLRKGTYVPKAVKPRHGR | |
| | SAWLEIPERMLEQKPVEIYRGDLVEIDGKNGCFVGYNIANGSWTFVDPLT | |
| | REEIDMPSLASLSSQTNLKVVRRGLLPMD | |
| BDLP Type II | KLEFATEMIPNPLLGLDKRTADGSEFESPKKKRKVGSVEVKTEAVGRLGR | 51 |
| Cas | PRYLVLGMDPGIASCGFALLDVNNHEILEMGVRLFDAPLHPKTKQSLAAI | |
| mammalian | RRGFRSTRRNIDRTQDRLKHCLKCFKKYGVVPDDADKEYLHMVKGDKR | |
| expression | PLELRVDGLNRMLTNREWGMVLYSLCKRRGYIPHGEGAQDKSSDDGKV | |
| construct | LAAISANEKEIAAGEFKTVGEWLASRPASRNHGGNYKMCVKHEQLIEET | |
| (includes N- | RILFEAQRSFGSKVASEDFEREYIDICNWERPRDDFDRRTYGLVGSCVY | |
| terminal SV5 | FPQEKRAARCTLTSELVSAYGALGNITIVLGDGGTRRLTAAERNECICVLF | |
| tag and NLS | TADPNAKTRVVKFGALRKKLDLGSRDYFKGVPADDEKTREVFKPSGWR | |
| and C- | ALYSTLRESNPQLLARLKNDIDLADAVMEAAAYASSASSLDNCLSQLPLD | |
| terminal NLS) | DDEKQALCALPYSSKVLNGYGSRSKKALDLLLACLEEPEVLNLTDAEAAC | |
| (aa) | GLDAMRLGGPQLERSEKLIPYEAWVSATGRTNNNPVVIRAMSQMRKVV | |
| | NAICGKWGVPNEIHVELDRELRLPKRAKEEIAKANRKNEKERERIAIQAA | |
| | DILSCSPDEVSGKQIEKYRLWEEQENSDLYTGERIHVQRLLNDDTYTQID | |
| | HILPFSRTGENSRNNKVLVLSESNQQKREQTPYEWMNSGHPGAPSWDE | |
| | FKLRVQANQHLKHRKKEFLLEQDLESKEVEFLQRNVTDTAYMSREVCAY | |
| | LSDCLLFPDDGMKVHVVPTTGRATAWLRRRWGLNFGATGEKDRSDDR | |
| | HHATDACVIAACSRSLVIKTAKVMKGTHWSVTRHMTPEERRDARMEALR | |
| | ESMPWESFANDVRDRRDSVVPTRYVPRKGSGELFEQTVYSYAGEVDSL | |
| | SRQVIGKSGGKASPKANVFILDDGKSALLVGDMLCLRLWFDPEVKKKGK | |
| | WVGGWYANPLYKADLPDLRKGTYVPKAVKPRHGRSAWLEIPERMLEQK | |
| | PVEIYRGDLVEIDGKNGCFVGYNIANGSWTFVDPLTREEIDMPSLASLSS | |
| | QTNLKVVRRGLLPMDSRKRTADGSEFESPKKKRKV | |
| BDLP Type II | ATGGTCGAGGTTAAAACAGAGGCGGTCGGTCGGCTCGGTCGTCCTC | |
| Cas coding | GATATCTGGTTTTGGGCATGGATCCGGGCATTGCGAGTTGCGGCTTC | |
| sequence (nt) | GCGTTGCTCGACGTCAACAATCATGAGATCTTGGAAATGGGCGTTCG | 52 |
| (not codon | TTTGTTCGATGCTCCGTTGCATCCTAAAACGAAGCAGAGCTTGGCCG | |
| optimized) | CCATTCGTCGCGGCTTCCGTTCGACGCGGCGTAATATCGACCGAACG | |
| | CAAGATCGACTCAAGCATTGCCTCAAGTGCTTTAAGAAATACGGTGTC | |
| | GTTCCGGATGATGCCGATAAAGAGTACCTTCATATGGTCAAGGGGGA | |
| | TAAACGGCCCCTCGAGCTTCGCGTCGACGGCCTGAACCGCATGCTC | |
| | ACGAACAGGGAGTGGGGCATGGTCCTTTATTCCCTTTGCAAGCGTCG | |
| | CGGCTACATTCCGCACGGCGAAGGCGCTCAGGATAAATCGAGCGAT | |
| | GACGGCAAAGTTCTTGCCGCGATCAGCGCGAATGAAAAAGAAATCGC | |
| | CGCAGGTGAATTCAAAACCGTAGGCGAATGGCTTGCGTCTCGTCCCG | |
| | CTAGCAGGAACCACGGGGGCAATTACAAGATGTGCGTCAAGCATGA | |
| | GCAGCTTATCGAAGAAACCCGTATTCTGTTCGAAGCACAGCGCTCGT | |
| | TCGGTTCCAAGGTTGCTTCTGAAGATTTCGAGCGCGAATACATCGAC | |
| | ATATGCAATTGGGAACGGCCGCGCGACGATTTCGATAGGCGCACGTA | |
| | CGGCCTGGTGGGGTCATGCGTTTACTTTCCACAAGAGAAGCGCGCG | |
| | GCACGCTGCACGCTTACAAGCGAGCTTGTTTCTGCGTATGGCGCTTT | |
| | GGGAAATATAACCATCGTGCTCGGCGATGGCGGCACACGCCGTCTTA | |
| | CGGCGGCCGAACGAAACGAATGCATCTGCGTATTGTTTACGGCCGAT | |
| | CCGAATGCGAAGACCCGCGTTGTGAAGTTCGGCGCCTTGCGCAAAA | |
| | AGCTCGACTTGGGTTCGCGTGATTATTTCAAGGGCGTTCCTGCCGAT | |
| | GACGAGAAGACCCGCGAGGTGTTCAAGCCCAGCGGTTGGCGCGCCT | |
| | TGTATTCGACTTTGAGGGAATCTAATCCGCAGCTTCTCGCAAGGCTG | |
| | AAGAATGATATCGATTTGGCGGATGCCGTTATGGAGGCCGCCGCGTA | |
| | TGCGTCGTCTGCATCGAGCTTGGACAATTGCCTCAGCCAGCTTCCTC | |
| | TTGATGACGATGAAAAACAGGCGCTTTGCGCGCTGCCGTATTCGTCT | |
| | AAAGTGCTGAATGGATACGGAAGTCGTTCAAAGAAAGCGCTCGACTT | |
| | GCTGCTTGCTTGCCTTGAAGAGCCTGAAGTTCTTAATCTGACCGATG | |
| | CAGAGGCGGCATGCGGGTTGGATGCGATGCGTCTTGGCGGTCCTCA | |
| | GCTCGAACGCTCTGAGAAACTAATTCCATATGAGGCTTGGGTAAGCG | |
| | CGACGGGTCGCACGAATAATAATCCTGTCGTCATTCGCGCTATGTCG | |
| | CAAATGCGCAAGGTCGTAAATGCCATATGCGGCAAATGGGGCGTTCC | |
| | GAACGAAATCCACGTTGAGCTTGACCGCGAGCTTCGGTTGCCCAAGC | |
| | GCGCGAAAGAAGAAATCGCTAAGGCCAATAGGAAAAACGAGAAAGAG | |
| | CGCGAGCGCATAGCCATTCAGGCTGCGGACATTCTTTCCTGCAGCCC | |
| | CGATGAGGTGAGTGGGAAACAGATAGAGAAGTATCGCTTGTGGGAG | |
| | GAGCAGGAAAACTCCGATTTGTATACGGGTGAGCGCATTCATGTCCA | |
| | ACGCCTTTTGAACGATGACACCTATACGCAGATTGACCATATTCTTCC | |
| | GTTTTCGCGAACGGGAGAGAATTCGCGCAATAACAAAGTGCTCGTGC | |
| | TTTCGGAGAGCAATCAGCAGAAACGCGAGCAGACTCCGTATGAATGG | |
| | ATGAATTCGGGCCATCCTGGCGCTCCGTCGTGGGACGAGTTCAAGCT | |
| | TCGAGTTCAGGCGAATCAGCACCTTAAACATCGTAAGAAAGAATTCTT | |
| | GCTCGAACAGGATTTGGAATCGAAGGAAGTCGAATTTCTCCAGCGAA | |
| | ACGTGACCGACACGGCCTATATGTCGCGCGAAGTCTGCGCCTATCTA | |
| | TCCGATTGTCTGCTGTTCCCCGATGATGGCATGAAGGTTCACGTTGT | |
| | CCCCACGACGGGTCGTGCTACGGCATGGTTGCGTCGCCGATGGGGG | |
| | CTGAACTTCGGAGCAACGGGGGAGAAGGATCGTTCGGACGATCGTC | |
| | ATCATGCCACGGATGCTTGCGTTATTGCCGCATGCAGCAGGAGCCTC | |
| | GTCATCAAGACGGCAAAGGTCATGAAGGGAACGCATTGGAGTGTTAC | |
| | GCGTCATATGACGCCGGAGGAGCGCAGAGATGCTCGTATGGAGGCG | |
| | TTGCGTGAGAGCATGCCCTGGGAAAGCTTTGCCAACGATGTCCGCGA | |
| | TCGTCGCGATTCCGTCGTGCCCACGCGCTATGTGCCTCGCAAAGGC | |
| | AGCGGTGAGCTTTTCGAGCAGACGGTTTATTCGTATGCGGGAGAAGT | |
| | AGATTCGTTGAGTCGGCAAGTAATTGGCAAGTCGGGTGGCAAAGCAT | |
| | CTCCGAAGGCGAATGTTTTCATCCTTGATGATGGAAAATCAGCACTGT | |
| | TGGTTGGCGACATGCTTTGCCTGAGACTGTGGTTTGATCCGGAAGTG | |
| | AAGAAGAAAGGCAAAGTTGTTGGAGGTTGGTATGCTAATCCACTGTA | |
| | CAAAGCCGATCTTCCAGACCTGCGGAAGGGTACGTATGTCCCGAAG | |
| | GCGGTTAAGCCAAGGCATGGAAGGTCTGCATGGCTTGAGATTCCAGA | |
| | ACGCATGCTTGAGCAGAAGCCGGTAGAGATATACCGTGGAGACTTGG | |
| | TTGAAATTGATGGAAAGAATGGATGCTTTGTTGGATACAACATCGCTA | |
| | ACGGATCATGGACCTTTGTTGACCCTCTAACGCGCGAGGAGATTGAC | |
| | ATGCCTTCCCTGGCCAGCCTTTCGTCGCAAACGAACTTGAAGGTCGT | |
| | GCGAAGAGGGCTGCTTCCCATGGATTGA | |
| BDLP Type II | GTCGAGGTGAAAACCGAGGCCGTGGGCAGACTGGGCAGACCTAGAT | 53 |
| Cas coding | ACCTGGTGCTGGGCATGGACCCCGGCATCGCTTCCTGCGGATTCGC | |
| sequence (nt) | CCTGCTGGATGTTAACAATCACGAGATCCTGGAGATGGGCGTGCGG | |
| (human | CTGTTTGATGCTCCTCTGCACCCCAAAACTAAGCAGAGCCTGGCCGC | |
| codon- | CATCAGGCGCGGATTTAGATCCACTAGAAGAAACATTGACAGAACCC | |
| optimized) | AGGACAGACTGAAACACTGCCTGAAGTGCTTCAAGAAATACGGCGTG | |
| | GTTCCAGATGACGCTGACAAGGAATACCTGCACATGGTGAAGGGGG | |
| | ATAAGCGGCCTCTGGAACTGAGAGTGGACGGCCTGAACCGGATGCT | |
| | GACAAACAGAGAGTGGGGAATGGTTCTGTACTCTCTGTGCAAGAGAA | |
| | GAGGCTACATCCCACACGGAGAGGGCGCCCAGGACAAGAGCTCCGA | |
| | CGACGGCAAGGTGCTGGCCGCTATCAGCGCCAACGAGAAAGAAATC | |
| | GCAGCCGGAGAGTTCAAGACAGTGGGCGAGTGGCTGGCCAGCAGAC | |
| | CTGCCAGCAGAAACCACGGCGGCAATTACAAAATGTGCGTGAAGCAC | |
| | GAGCAACTCATCGAGGAGACAAGAATCCTGTTCGAGGCCCAGCGGT | |
| | CTTTTGGCAGCAAGGTCGCCTCCGAGGATTTCGAAAGAGAGTACATC | |
| | GATATCTGCAACTGGGAAAGACCTAGAGATGACTTCGATAGACGGAC | |
| | ATACGGCCTGGTGGGCAGCTGTGTGTACTTCCCTCAGGAGAAGCGG | |
| | GCCGCCAGATGCACCCTGACCAGCGAGCTGGTCAGCGCTTATGGCG | |
| | CCCTGGGCAACATCACCATCGTGCTGGGCGACGGCGGCACCAGACG | |
| | GCTTACAGCCGCTGAGAGAAATGAGTGCATCTGTGTGCTGTTCACAG | |
| | CCGATCCCAACGCCAAGACCCGGGTGGTGAAGTTCGGCGCTCTTAG | |
| | AAAGAAGCTGGACCTGGGCAGTAGAGACTACTTCAAGGGCGTGCCC | |
| | GCTGATGACGAAAAGACCAGAGAAGTGTTCAAGCCTTCTGGCTGGCG | |
| | GGCTCTGTACAGCACCCTGCGGGAGAGCAACCCCCAGCTGCTGGCC | |
| | AGACTGAAGAATGACATCGATCTCGCCGACGCCGTGATGGAAGCCG | |
| | CCGCCTACGCCAGCAGCGCCTCTAGCCTGGACAACTGCCTGAGTCA | |
| | GCTTCCCCTGGACGATGACGAGAAGCAAGCCCTGTGCGCCCTGCCT | |
| | TACAGCAGCAAGGTGCTGAACGGCTACGGCAGCAGAAGCAAGAAAG | |
| | CCCTGGACCTGCTGCTGGCCTGCCTGGAGGAGCCTGAGGTGCTGAA | |
| | CCTGACAGATGCCGAGGCCGCTTGTGGACTGGATGCTATGAGACTG | |
| | GGAGGTCCTCAGCTGGAACGGAGCGAGAAGTTGATCCCCTATGAGG | |
| | CCTGGGTGAGCGCCACAGGTAGAACCAACAACAACCCCGTGGTGAT | |
| | CCGGGCTATGAGCCAGATGCGGAAAGTGGTGAACGCCATCTGCGGC | |
| | AAGTGGGGCGTGCCTAACGAGATCCACGTGGAACTGGATAGAGAGC | |
| | TGAGACTGCCTAAGCGCGCCAAAGAGGAAATCGCCAAGGCCAACCG | |
| | GAAGAACGAGAAAGAAAGAGAACGGATCGCCATCCAGGCCGCCGAC | |
| | ATCCTGTCTTGCAGCCCTGACGAGGTGAGCGGCAAACAGATCGAGAA | |
| | GTACAGACTGTGGGAGGAACAGGAGAACAGCGACCTGTACACCGGC | |
| | GAAAGAATCCACGTGCAGAGACTACTAAATGACGACACCTATACACA | |
| | GATTGACCACATCCTGCCCTTCAGCAGAACAGGCGAAAACAGCAGAA | |
| | ACAACAAGGTGCTTGTGCTCAGCGAATCTAACCAGCAGAAGAGAGAG | |
| | CAGACCCCATACGAGTGGATGAACAGCGGCCACCCTGGCGCCCCTT | |
| | CTTGGGACGAGTTCAAGCTGCGCGTTCAGGCCAATCAACACCTGAAG | |
| | CACAGAAAGAAGGAGTTCCTGCTGGAACAAGATCTGGAATCCAAGGA | |
| | GGTGGAGTTCCTGCAGAGAAACGTGACCGATACCGCCTACATGAGC | |
| | CGGGAGGTTTGTGCCTACCTGAGCGACTGCCTGCTGTTTCCGGACG | |
| | ACGGCATGAAAGTGCATGTGGTGCCTACCACCGGCAGAGCCACCGC | |
| | ATGGCTGAGAAGAAGATGGGGCCTGAACTTCGGCGCCACCGGAGAG | |
| | AAGGACAGGAGCGACGATAGACACCACGCCACAGACGCTTGTGTGA | |
| | TCGCCGCCTGTTCCCGGAGCCTCGTGATCAAGACCGCTAAGGTCATG | |
| | AAAGGAACCCACTGGTCCGTGACACGCCACATGACACCTGAGGAAC | |
| | GGAGGGATGCCAGAATGGAAGCCCTGAGGGAAAGCATGCCCTGGGA | |
| | GTCTTTCGCTAACGACGTGCGGGACAGAAGAGACAGCGTGGTGCCT | |
| | ACCCGGTACGTGCCTAGAAAGGGCTCTGGCGAGCTGTTCGAGCAGA | |
| | CAGTGTACAGCTACGCCGGCGAAGTGGATTCTCTGTCCAGACAGGTG | |
| | ATCGGAAAGAGCGGCGGCAAAGCCTCTCCTAAGGCCAACGTGTTCAT | |
| | CCTGGACGACGGCAAGAGCGCCCTGCTGGTCGGCGACATGCTGTGC | |
| | CTGAGACTGTGGTTCGACCCAGAAGTCAAGAAAAAGGGCAAGGTGGT | |
| | GGGTGGATGGTACGCGAATCCCCTGTATAAGGCCGACCTGCCTGAT | |
| | CTGCGGAAGGGCACATACGTGCCTAAGGCCGTGAAACCAAGACACG | |
| | GCAGAAGCGCCTGGCTGGAGATCCCCGAGAGAATGCTGGAACAGAA | |
| | ACCTGTGGAGATCTACCGGGGCGACCTGGTTGAAATCGATGGCAAG | |
| | AACGGTTGTTTTGTGGGATACAACATCGCTAATGGCTCTTGGACCTTC | |
| | GTGGACCCTCTTACCCGGGAAGAGATCGACATGCCTAGCCTGGCAA | |
| | GCCTGTCCAGCCAGACCAACCTGAAGGTGGTTAGACGCGGCCTGCT | |
| | GCCTATGGAC | |
| BDLP Type II | aagcttgaattcgccaccATGATCCCCAATCCCCTGCTGGGCCTGGACAAGC | 54 |
| Cas | GGACTGCCGACGGCAGTGAGTTCGAGTCTCCCAAGAAAAAGAGGAA | |
| mammalian | AGTGggatccGTCGAGGTGAAAACCGAGGCCGTGGGCAGACTGGGCA | |
| expression | GACCTAGATACCTGGTGCTGGGCATGGACCCCGGCATCGCTTCCTG | |
| construct | CGGATTCGCCCTGCTGGATGTTAACAATCACGAGATCCTGGAGATGG | |
| (includes N- | GCGTGCGGCTGTTTGATGCTCCTCTGCACCCCAAAACTAAGCAGAGC | |
| terminal SV5 | CTGGCCGCCATCAGGCGCGGATTTAGATCCACTAGAAGAAACATTGA | |
| tag and NLS | CAGAACCCAGGACAGACTGAAACACTGCCTGAAGTGCTTCAAGAAAT | |
| and C- | ACGGCGTGGTTCCAGATGACGCTGACAAGGAATACCTGCACATGGTG | |
| terminal NLS) | AAGGGGGATAAGCGGCCTCTGGAACTGAGAGTGGACGGCCTGAACC | |
| (nt) | GGATGCTGACAAACAGAGAGTGGGGAATGGTTCTGTACTCTCTGTGC | |
| | AAGAGAAGAGGCTACATCCCACACGGAGAGGGCGCCCAGGACAAGA | |
| | GCTCCGACGACGGCAAGGTGCTGGCCGCTATCAGCGCCAACGAGAA | |
| | AGAAATCGCAGCCGGAGAGTTCAAGACAGTGGGCGAGTGGCTGGCC | |
| | AGCAGACCTGCCAGCAGAAACCACGGCGGCAATTACAAAATGTGCGT | |
| | GAAGCACGAGCAACTCATCGAGGAGACAAGAATCCTGTTCGAGGCC | |
| | CAGCGGTCTTTTGGCAGCAAGGTCGCCTCCGAGGATTTCGAAAGAGA | |
| | GTACATCGATATCTGCAACTGGGAAAGACCTAGAGATGACTTCGATA | |
| | GACGGACATACGGCCTGGTGGGCAGCTGTGTGTACTTCCCTCAGGA | |
| | GAAGCGGGCCGCCAGATGCACCCTGACCAGCGAGCTGGTCAGCGCT | |
| | TATGGCGCCCTGGGCAACATCACCATCGTGCTGGGCGACGGCGGCA | |
| | CCAGACGGCTTACAGCCGCTGAGAGAAATGAGTGCATCTGTGTGCTG | |
| | TTCACAGCCGATCCCAACGCCAAGACCCGGGTGGTGAAGTTCGGCG | |
| | CTCTTAGAAAGAAGCTGGACCTGGGCAGTAGAGACTACTTCAAGGGC | |
| | GTGCCCGCTGATGACGAAAAGACCAGAGAAGTGTTCAAGCCTTCTGG | |
| | CTGGCGGGCTCTGTACAGCACCCTGCGGGAGAGCAACCCCCAGCTG | |
| | CTGGCCAGACTGAAGAATGACATCGATCTCGCCGACGCCGTGATGG | |
| | AAGCCGCCGCCTACGCCAGCAGCGCCTCTAGCCTGGACAACTGCCT | |
| | GAGTCAGCTTCCCCTGGACGATGACGAGAAGCAAGCCCTGTGCGCC | |
| | CTGCCTTACAGCAGCAAGGTGCTGAACGGCTACGGCAGCAGAAGCA | |
| | AGAAAGCCCTGGACCTGCTGCTGGCCTGCCTGGAGGAGCCTGAGGT | |
| | GCTGAACCTGACAGATGCCGAGGCCGCTTGTGGACTGGATGCTATG | |
| | AGACTGGGAGGTCCTCAGCTGGAACGGAGCGAGAAGTTGATCCCCT | |
| | ATGAGGCCTGGGTGAGCGCCACAGGTAGAACCAACAACAACCCCGT | |
| | GGTGATCCGGGCTATGAGCCAGATGCGGAAAGTGGTGAACGCCATC | |
| | TGCGGCAAGTGGGGCGTGCCTAACGAGATCCACGTGGAACTGGATA | |
| | GAGAGCTGAGACTGCCTAAGCGCGCCAAAGAGGAAATCGCCAAGGC | |
| | CAACCGGAAGAACGAGAAAGAAAGAGAACGGATCGCCATCCAGGCC | |
| | GCCGACATCCTGTCTTGCAGCCCTGACGAGGTGAGCGGCAAACAGA | |
| | TCGAGAAGTACAGACTGTGGGAGGAACAGGAGAACAGCGACCTGTA | |
| | CACCGGCGAAAGAATCCACGTGCAGAGACTACTAAATGACGACACCT | |
| | ATACACAGATTGACCACATCCTGCCCTTCAGCAGAACAGGCGAAAAC | |
| | AGCAGAAACAACAAGGTGCTTGTGCTCAGCGAATCTAACCAGCAGAA | |
| | GAGAGAGCAGACCCCATACGAGTGGATGAACAGCGGCCACCCTGGC | |
| | GCCCCTTCTTGGGACGAGTTCAAGCTGCGCGTTCAGGCCAATCAACA | |
| | CCTGAAGCACAGAAAGAAGGAGTTCCTGCTGGAACAAGATCTGGAAT | |
| | CCAAGGAGGTGGAGTTCCTGCAGAGAAACGTGACCGATACCGCCTA | |
| | CATGAGCCGGGAGGTTTGTGCCTACCTGAGCGACTGCCTGCTGTTTC | |
| | CGGACGACGGCATGAAAGTGCATGTGGTGCCTACCACCGGCAGAGC | |
| | CACCGCATGGCTGAGAAGAAGATGGGGCCTGAACTTCGGCGCCACC | |
| | GGAGAGAAGGACAGGAGCGACGATAGACACCACGCCACAGACGCTT | |
| | GTGTGATCGCCGCCTGTTCCCGGAGCCTCGTGATCAAGACCGCTAA | |
| | GGTCATGAAAGGAACCCACTGGTCCGTGACACGCCACATGACACCTG | |
| | AGGAACGGAGGGATGCCAGAATGGAAGCCCTGAGGGAAAGCATGCC | |
| | CTGGGAGTCTTTCGCTAACGACGTGCGGGACAGAAGAGACAGCGTG | |
| | GTGCCTACCCGGTACGTGCCTAGAAAGGGCTCTGGCGAGCTGTTCG | |
| | AGCAGACAGTGTACAGCTACGCCGGCGAAGTGGATTCTCTGTCCAGA | |
| | CAGGTGATCGGAAAGAGCGGCGGCAAAGCCTCTCCTAAGGCCAACG | |
| | TGTTCATCCTGGACGACGGCAAGAGCGCCCTGCTGGTCGGCGACAT | |
| | GCTGTGCCTGAGACTGTGGTTCGACCCAGAAGTCAAGAAAAAGGGCA | |
| | AGGTGGTGGGTGGATGGTACGCGAATCCCCTGTATAAGGCCGACCT | |
| | GCCTGATCTGCGGAAGGGCACATACGTGCCTAAGGCCGTGAAACCA | |
| | AGACACGGCAGAAGCGCCTGGCTGGAGATCCCCGAGAGAATGCTGG | |
| | AACAGAAACCTGTGGAGATCTACCGGGGCGACCTGGTTGAAATCGAT | |
| | GGCAAGAACGGTTGTTTTGTGGGATACAACATCGCTAATGGCTCTTG | |
| | GACCTTCGTGGACCCTCTTACCCGGGAAGAGATCGACATGCCTAGCC | |
| | TGGCAAGCCTGTCCAGCCAGACCAACCTGAAGGTGGTTAGACGCGG | |
| | CCTGCTGCCTATGGACtctagaAAGCGGACAGCAGACGGCTCCGAATTT | |
| | GAAAGCCCTAAGAAAAAGAGAAAGGTGTGA | |

In some embodiments an BDLP Type II Cas protein comprises an amino acid sequence of SEQ ID NO:49, SEQ ID NO:50 or SEQ ID:51. In some embodiments, a BDLP Type II Cas protein has nickase activity, for example resulting from one or more amino acid substitutions relative to the sequence of SEQ ID NO:49, SEQ ID NO:50, or SEQ ID:51. In some embodiments, the one or more amino acid substitutions providing nickase activity is a D23A Substitution, wherein the position of the D23A substitution is defined with respect to the amino acid numbering of SEQ ID NO:50. In some embodiments, the one or more amino acid substitutions providing nickase activity is a N629A Substitution, wherein the position of the N629A substitution is defined with respect to the amino acid numbering of SEQ ID NO:50. In some embodiments, a BDLP Type II Cas protein is catalytically inactive, for example due to both a D23A substitution and a N629A substitution, where the positions of the substitutions are defined with respect to the amino acid numbering of SEQ ID NO:50.

### 6.2.2.3. BDKL Type II Cas Proteins

In one aspect, the disclosure provides BDKL Type II Cas proteins. BDKL Type II Cas proteins can be further classified as Type IIC Cas proteins. The BDKL Type II Cas proteins typically comprise an amino acid sequence that is at least 50%, at least 55%, at least 60%, at least 65%, at least 70%, at least 75%, at least 85%, at least 90%, or at least 95% identical to SEQ ID NO:55. In some embodiments, the BDKL Type II Cas proteins comprise an amino acid sequence that is at least 95%, at least 96%, at least 97%, at least 98%, or at least 99% identical to SEQ ID NO:55. In some embodiments, a BDKL Type II Cas protein comprises an amino acid sequence that is identical to SEQ ID NO:55.

Exemplary BDKL Type II Cas protein sequences and nucleotide sequences encoding exemplary BDKL Type II Cas proteins are set forth in Table 2C.

TABLE 2C
BDKL Type II Cas Sequences

| | | SEQ ID |
|---|---|---|
| Name | Sequence | NO |
| BDKL Type II | PMAVNREVGKLGNPRYLVLGLDPGIASCGFALLDLNNHEILEMGSRLFD | 55 |
| Cas amino | APTVPKTGQSKAVVRRMHRSTRRNLDRTQDRLKHCLNALKEADLVPED | |
| acid | ATKEYFHTQKGDKPPLRLRVDGLDRRLTEREWAIVLYSLCKRRGYIPHG | |
| sequence | EGSVDGSSEDGKVLAAVSANAKLLDEGKYRTVGEWLASQERSRNRAGS | |
| (without N- | YEKCVTHKLLMEEASTLFAKQRGFGNEYASPELEKKYVGICDWEKPRDA | |
| terminal | FDERVYQTQVGHCIYFHDEKRAARSTLSNELVSAYAAFGNISIVQPGHET | |
| methionine) | RRVSPDVRDQCIGLLFSASPDGPASGTKVTYKSLRKLLDLSAKASFKGIN | |
| | VDDEKSREVFQPKGWRALRATLVDSAPELLMRLRTDIDLADEVFEAAAY | |
| | SSSIEVLRNRLGLLDLSDEEVDKLSRLPYASRVLNGYGSRSKKALDILLD | |
| | QFEDPEILDLAEAERSSGLLGKRMAGNGIAKSDRLMPYKCWLEDTGRTN | |
| | NNPVVLRAIAQMRKVVNAICREWGVPHEIHVELDRELKLSKKQQDKIRKN | |
| | NKKNEAENARVAKQIAGLLGCDPGSVSRNQIRKYTLWEQQDNVDIYTGD | |
| | AIDLERLLADSMYTQIDHVLPFSRTGDNAWYNKVLCLASSNQHKRERSP | |
| | YEWMTSGEAGAPSWEEFQARVVENHKLGYKKRSFLLEENLDAKVDDFL | |
| | RRSITDTAYMSRELSAYLDDCLEFPEGRTKVPVQPTNGRATAWLRHAW | |
| | GLNFGAQGEKDREDDRHHATDACVIAACNRSMLIKTAQYSSGKRKMSE | |
| | EQKDAELREAQPWPTFSKDVTTRRDCVIPTRFVPHKAGGEIFEQTLYAY | |
| | KGTNAQGKHILAKTDASTGKEKETVAGNAVVSVDGKSAVIVSEMLCLML | |
| | WHDADARPNGKVKGQWYADPIYRADLPALRDGTFVHRIAKAHKGRDVW | |
| | PEIPDRVLENTKPIVVYRGDVLMVDDAIGRFAGIDIDGVRWEFAQISPDVP | |
| | AEIDTIGSLGNENKVKLVTEDVLGHCWGQLNMKVGCAE | |
| BDKL Type II | MPMAVNREVGKLGNPRYLVLGLDPGIASCGFALLDLNNHEILEMGSRLF | 56 |
| Cas amino | DAPTVPKTGQSKAVVRRMHRSTRRNLDRTQDRLKHCLNALKEADLVPE | |
| acid | DATKEYFHTQKGDKPPLRLRVDGLDRRLTEREWAIVLYSLCKRRGYIPH | |
| sequence | GEGSVDGSSEDGKVLAAVSANAKLLDEGKYRTVGEWLASQERSRNRA | |
| | GSYEKCVTHKLLMEEASTLFAKQRGFGNEYASPELEKKYVGICDWEKPR | |
| | DAFDERVYQTQVGHCIYFHDEKRAARSTLSNELVSAYAAFGNISIVQPGH | |
| | ETRRVSPDVRDQCIGLLFSASPDGPASGTKVTYKSLRKLLDLSAKASFKG | |
| | INVDDEKSREVFQPKGWRALRATLVDSAPELLMRLRTDIDLADEVFEAAA | |
| | YSSSIEVLRNRLGLLDLSDEEVDKLSRLPYASRVLNGYGSRSKKALDILLD | |
| | QFEDPEILDLAEAERSSGLLGKRMAGNGIAKSDRLMPYKCWLEDTGRTN | |
| | NNPWVLRAIAQMRKVVNAICREWGVPHEIHVELDRELKLSKKQQDKIRKN | |
| | NKKNEAENARVAKQIAGLLGCDPGSVSRNQIRKYTLWEQQDNVDIYTGD | |
| | AIDLERLLADSMYTQIDHVLPFSRTGDNAWYNKVLCLASSNQHKRERSP | |
| | YEWMTSGEAGAPSWEEFQARVVENHKLGYKKRSFLLEENLDAKVDDFL | |
| | RRSITDTAYMSRELSAYLDDCLEFPEGRTKVPVQPTNGRATAWLRHAW | |
| | GLNFGAQGEKDREDDRHHATDACVIAACNRSMLIKTAQYSSGKRKMSE | |
| | EQKDAELREAQPWPTFSKDVTTRRDCVIPTRFVPHKAGGEIFEQTLYAY | |
| | KGTNAQGKHILAKTDASTGKEKETVAGNAVVSVDGKSAVIVSEMLCLML | |
| | WHDADARPNGKVKGQWYADPIYRADLPALRDGTFVHRIAKAHKGRDVW | |
| | PEIPDRVLENTKPIVVYRGDVLMVDDAIGRFAGIDIDGVRWEFAQISPDVP | |
| | AEIDTIGSLGNENKVKLVTEDVLGHCWGQLNMKVGCAE | |
| BDKL Type II | MIPNPLLGLDKRTADGSEFESPKKKRKVGSPMAVNREVGKLGNPRYLVL | 57 |
| Cas | GLDPGIASCGFALLDLNNHEILEMGSRLFDAPTVPKTGQSKAVVRRMHR | |
| mammalian | STRRNLDRTQDRLKHCLNALKEADLVPEDATKEYFHTQKGDKPPLRLRV | |
| expression | DGLDRRLTEREWAIVLYSLCKRRGYIPHGEGSVDGSSEDGKVLAAVSAN | |
| construct | AKLLDEGKYRTVGEWLASQERSRNRAGSYEKCVTHKLLMEEASTLFAK | |
| (includes N- | QRGFGNEYASPELEKKYVGICDWEKPRDAFDERVYQTQVGHCIYFHDE | |
| terminal SV5 | KRAARSTLSNELVSAYAAFGNISIVQPGHETRRVSPDVRDQCIGLLFSAS | |
| tag and NLS | PDGPASGTKVTYKSLRKLLDLSAKASFKGINVDDEKSREVFQPKGWRAL | |
| and C- | RATLVDSAPELLMRLRTDIDLADEVFEAAAYSSSIEVLRNRLGLLDLSDEE | |
| terminal NLS) | VDKLSRLPYASRVLNGYGSRSKKALDILLDQFEDPEILDLAEAERSSGLL | |
| (aa) | GKRMAGNGIAKSDRLMPYKCWLEDTGRTNNNPVVLRAIAQMRKVVNAI | |
| | CREWGVPHEIHVELDRELKLSKKQQDKIRKNNKKNEAENARVAKQIAGL | |
| | LGCDPGSVSRNQIRKYTLWEQQDNVDIYTGDAIDLERLLADSMYTQIDHV | |
| | LPFSRTGDNAWYNKVLCLASSNQHKRERSPYEWMTSGEAGAPSWEEF | |
| | QARVVENHKLGYKKRSFLLEENLDAKVDDFLRRSITDTAYMSRELSAYLD | |
| | DCLEFPEGRTKVPVQPTNGRATAWLRHAWGLNFGAQGEKDREDDRHH | |
| | ATDACVIAACNRSMLIKTAQYSSGKRKMSEEQKDAELREAQPWPTFSKD | |
| | VTTRRDCVIPTRFVPHKAGGEIFEQTLYAYKGTNAQGKHILAKTDASTGK | |
| | EKETVAGNAVVSVDGKSAVIVSEMLCLMLWHDADARPNGKVKGQWYA | |
| | DPIYRADLPALRDGTFVHRIAKAHKGRDVWPEIPDRVLENTKPIVVYRGD | |
| | VLMVDDAIGRFAGIDIDGVRWEFAQISPDVPAEIDTIGSLGNENKVKLVTE | |
| | DVLGHCWGQLNMKVGCAESRKRTADGSEFESPKKKRKV | |
| BDKL Type II | ATGCCCATGGCCGTCAACCGTGAAGTCGGCAAGTTGGGGAATCCTC | 58 |
| Cas coding | GCTATTTGGTGCTTGGTCTCGACCCGGGGATTGCCAGCTGCGGGTTT | |
| sequence (nt) | GCTCTCCTTGACCTCAACAATCACGAGATCCTCGAGATGGGGTCGCG | |
| (not codon | CCTATTTGACGCCCCGACAGTTCCTAAGACCGGCCAGAGCAAGGCG | |
| optimized) | GTCGTGAGGCGTATGCATCGCTCGACCCGCCGCAACCTCGACAGGA | |
| | CCCAAGACCGTCTCAAGCATTGTCTCAATGCCCTAAAGGAAGCCGAC | |
| | TTGGTCCCGGAGGATGCCACCAAGGAGTACTTCCATACGCAGAAGG | |
| | GTGACAAGCCGCCGCTGCGGCTCAGAGTCGACGGGCTCGATAGGAG | |
| | GCTGACAGAGCGCGAATGGGCCATCGTGCTCTATTCCCTGTGCAAGC | |
| | GTCGTGGCTACATTCCCCATGGTGAGGGGAGCGTTGATGGCTCCAG | |
| | CGAGGACGGCAAGGTGCTGGCTGCCGTCTCTGCCAATGCCAAGCTG | |
| | TTGGACGAGGGAAAGTATCGCACGGTAGGTGAGTGGCTGGCTTCGC | |
| | AGGAGCGCAGTCGCAATAGGGCCGGATCGTATGAGAAGTGCGTCAC | |
| | CCACAAGCTGCTGATGGAAGAGGCCTCGACGCTCTTTGCCAAGCAGA | |
| | GGGGCTTCGGCAACGAATACGCATCGCCGGAACTCGAGAAGAAATAT | |
| | GTCGGGATCTGCGATTGGGAAAAGCCGCGAGATGCCTTCGATGAGC | |
| | GCGTCTATCAGACGCAAGTTGGCCATTGCATCTATTTTCATGATGAGA | |
| | AACGCGCCGCTCGGTCCACGCTTTCCAACGAGCTCGTTTCCGCCTAT | |
| | GCGGCCTTTGGCAACATCTCGATAGTCCAGCCTGGTCATGAGACGCG | |
| | TCGGGTCAGTCCTGATGTTCGCGACCAGTGCATCGGCTTGCTGTTCT | |
| | CGGCGTCACCCGATGGCCCAGCCTCAGGCACTAAGGTCACCTATAA | |
| | GAGCCTGAGGAAGCTCCTGGACCTCTCCGCCAAGGCCTCCTTTAAAG | |
| | GAATCAATGTCGATGACGAGAAATCGCGCGAGGTCTTTCAGCCAAAG | |
| | GGCTGGCGAGCCCTGAGGGCTACGTTGGTCGATAGCGCCCCCGAGC | |
| | TCCTCATGAGGCTCAGGACGGACATCGATCTTGCCGATGAGGTCTTC | |
| | GAGGCGGCGGCTTACTCGTCATCCATCGAGGTTCTCAGGAACCGGC | |
| | TTGGCCTGCTCGACCTTTCTGACGAGGAAGTCGACAAGCTGAGCCGA | |
| | CTGCCGTATGCGAGCAGGGTCCTCAACGGCTATGGCTCTAGGTCGA | |
| | AGAAGGCATTGGACATCCTCTTGGATCAGTTTGAAGATCCAGAGATC | |
| | CTTGACCTCGCCGAGGCAGAGAGGAGCTCGGGGCTTCTTGGGAAGC | |
| | GGATGGCCGGGAACGGCATCGCCAAGTCCGATCGCCTGATGCCTTA | |
| | TAAGTGCTGGCTCGAAGACACTGGACGTACGAACAACAATCCTGTCG | |
| | TTCTGCGCGCAATCGCCCAGATGCGCAAGGTCGTCAACGCCATATGC | |
| | CGAGAGTGGGGGGTCCCGCATGAGATACATGTGGAGCTCGATAGGG | |
| | AATTAAAGCTCTCGAAGAAGCAACAAGACAAGATACGGAAAAACAACA | |
| | AGAAGAACGAGGCGGAGAACGCGCGCGTTGCCAAGCAGATTGCCGG | |
| | TCTGCTTGGGTGCGACCCGGGCTCCGTGAGCAGGAACCAGATTCGC | |
| | AAATACACCCTGTGGGAGCAGCAGGACAACGTCGACATCTATACGGG | |
| | GGACGCAATCGATCTCGAGCGCCTGCTCGCAGACAGCATGTACACC | |
| | CAAATAGACCACGTCTTGCCGTTCTCGAGGACTGGCGACAACGCTTG | |
| | GTACAACAAGGTTCTCTGCCTTGCATCGAGCAACCAGCACAAGCGAG | |
| | AGCGCTCACCTTATGAGTGGATGACGTCTGGCGAAGCCGGGGCTCC | |
| | CTCTTGGGAGGAGTTTCAGGCTCGTGTGGTCGAGAATCACAAGCTTG | |
| | GCTATAAGAAGCGCTCGTTCCTTCTCGAAGAGAACCTCGACGCCAAG | |
| | GTCGATGACTTTCTTAGAAGGAGCATCACTGACACCGCATACATGTCT | |
| | CGCGAACTGAGCGCGTATCTCGACGACTGCCTCGAGTTCCCTGAGG | |
| | GGAGAACGAAGGTCCCTGTCCAGCCAACGAACGGCAGGGCAACCGC | |
| | GTGGTTGCGGCATGCTTGGGGGCTGAACTTCGGCGCGCAAGGCGAG | |
| | AAGGACCGGGAGGACGACCGCCATCACGCCACCGATGCTTGCGTGA | |
| | TCGCTGCCTGCAACCGCTCAATGCTCATCAAGACGGCGCAGTATTCC | |
| | AGCGGAAAGCGCAAGATGAGCGAGGAGCAGAAGGATGCCGAGCTGC | |
| | GGGAAGCCCAGCCCTGGCCAACGTTCTCCAAGGATGTGACAACAAG | |
| | GCGGGATTGCGTTATTCCCACCAGGTTCGTACCTCATAAGGCAGGAG | |
| | GAGAGATCTTCGAGCAAACGCTTTATGCCTATAAGGGAACGAACGCC | |
| | CAAGGCAAGCACATCCTTGCCAAGACCGATGCGTCAACCGGCAAGG | |
| | AAAAGGAAACGGTTGCCGGGAATGCCGTTGTTTCTGTAGATGGGAAG | |
| | TCGGCGGTCATCGTCAGCGAGATGCTTTGCTTGATGCTGTGGCATGA | |
| | CGCAGACGCACGGCCCAACGGCAAGGTCAAAGGCCAGTGGTATGCC | |
| | GACCCCATCTATCGCGCCGATTTGCCTGCTCTGCGAGACGGCACCTT | |
| | CGTCCATCGCATTGCAAAGGCGCATAAAGGAAGGGACGTTTGGCCTG | |
| | AGATACCCGACCGTGTGTTGGAGAACACCAAACCGATAGTCGTCTAT | |
| | CGAGGCGACGTCCTCATGGTGGATGATGCGATAGGACGATTTGCAG | |
| | GCATTGATATCGATGGTGTTAGGTGGGAATTCGCTCAAATATCTCCTG | |
| | ATGTTCCCGCAGAGATTGACACCATAGGAAGCCTCGGCAACGAGAAC | |
| | AAGGTGAAGCTGGTTACAGAGGATGTCCTTGGCCACTGTTGGGGCCA | |
| | ATTAAATATGAAAGTGGGCTGCGCAGAATAA | |
| BDKL Type II | CCTATGGCCGTGAATCGGGAAGTGGGAAAGCTGGGCAATCCTAGATA | 59 |
| Cas coding | CCTGGTGCTGGGCCTGGACCCTGGCATCGCCAGCTGCGGCTTCGCC | |
| sequence (nt) | CTGCTGGATCTGAACAATCACGAAATCCTTGAGATGGGCAGCAGACT | |
| (human | GTTCGACGCACCAACAGTGCCTAAGACCGGCCAGAGCAAGGCCGTG | |
| codon- | GTGCGGAGAATGCACCGCTCTACAAGAAGAAACCTGGATAGGACACA | |
| optimized) | GGACCGGCTGAAGCACTGTCTGAATGCCCTCAAGGAAGCCGATCTG | |
| | GTTCCAGAGGACGCGACAAAGGAATACTTCCACACCCAAAAGGGCGA | |
| | TAAGCCTCCACTGAGGCTGAGAGTGGACGGCCTGGACCGGCGGCTG | |
| | ACCGAGAGAGAATGGGCCATCGTGCTGTACAGCCTGTGCAAGAGAA | |
| | GAGGCTACATCCCCCACGGCGAAGGCTCTGTGGATGGCAGCAGCGA | |
| | GGACGGCAAGGTCTTGGCCGCCGTGAGCGCCAACGCTAAGCTGCTG | |
| | GACGAGGGAAAATACAGAACCGTGGGAGAATGGCTGGCCAGTCAGG | |
| | AGAGATCCCGGAACAGAGCCGGCTCCTACGAGAAATGCGTGACACA | |
| | CAAACTGCTGATGGAAGAGGCTTCTACTCTTTTTGCCAAGCAAAGAG | |
| | GCTTCGGCAACGAGTATGCCAGCCCTGAATTGGAAAAGAAGTACGTG | |
| | GGTATTTGTGATTGGGAGAAGCCCAGAGATGCATTTGACGAACGCGT | |
| | CTACCAGACACAAGTGGGACATTGCATCTACTTTCACGATGAAAAGA | |
| | GAGCTGCCAGAAGCACGCTGAGCAACGAGCTGGTGTCGGCTTACGC | |
| | AGCTTTCGGCAACATCTCTATCGTGCAGCCTGGCCACGAAACCAGAC | |
| | GGGTCAGCCCCGACGTTCGGGACCAGTGCATCGGCCTGCTGTTCAG | |
| | CGCCTCCCCAGACGGTCCTGCCAGCGGCACAAAGGTGACCTACAAA | |
| | AGCCTGAGAAAGCTGCTGGACCTGTCTGCTAAGGCCTCTTTTAAGGG | |
| | CATCAACGTGGACGACGAGAAGAGCCGGGAAGTTTTCCAACCCAAG | |
| | GGCTGGAGAGCTCTGAGGGCCACCCTGGTGGACAGCGCCCCTGAG | |
| | CTGCTGATGAGACTCAGAACAGACATCGACCTGGCCGACGAGGTGTT | |
| | CGAGGCTGCCGCTTACAGCTCTTCTATCGAGGTGCTGAGAAATAGGC | |
| | TGGGCTTGCTGGATCTGAGCGACGAGGAAGTGGACAAGCTGTCCAG | |
| | ACTGCCATACGCCAGCAGAGTGCTGAACGGCTACGGCAGCCGGTCC | |
| | AAGAAGGCCCTGGACATCCTGCTGGACCAGTTCGAGGACCCTGAAAT | |
| | CCTGGACCTGGCTGAAGCCGAACGGAGCAGCGGCCTGCTCGGCAAG | |
| | CGGATGGCGGGCAACGGCATCGCTAAAAGCGACAGACTGATGCCTT | |
| | ACAAGTGCTGGCTGGAGGACACCGGCAGAACAAACAACAACCCTGT | |
| | GGTCCTGCGGGCCATCGCCCAGATGCGCAAGGTGGTGAACGCCATT | |
| | TGCAGAGAGTGGGGCGTGCCTCATGAGATCCACGTGGAACTGGATA | |
| | GAGAACTGAAGCTGAGCAAAAAGCAGCAGGACAAGATCCGGAAGAA | |
| | CAACAAGAAGAACGAGGCCGAAAACGCCCGGGTGGCCAAGCAGATC | |
| | GCCGGCCTGCTGGGCTGTGACCCCGGCAGCGTTAGCAGAAATCAGA | |
| | TCAGAAAGTACACCCTGTGGGAGCAGCAAGATAATGTGGACATCTAC | |
| | ACCGGAGATGCCATCGATCTGGAACGGCTCCTGGCTGACAGTATGTA | |
| | CACCCAGATCGACCACGTGCTCCCCTTCAGCAGAACCGGCGACAAC | |
| | GCCTGGTACAACAAAGTGCTCTGCCTGGCCAGCTCCAACCAGCACAA | |
| | GCGGGAGCGGTCACCCTACGAGTGGATGACCAGCGGCGAGGCCGG | |
| | AGCCCCTAGCTGGGAAGAGTTCCAGGCCCGAGTGGTGGAAAACCAC | |
| | AAGCTGGGATACAAGAAAAGATCCTTCCTGCTGGAAGAGAATCTGGA | |
| | TGCCAAGGTGGACGACTTCCTGAGAAGAAGCATCACCGACACCGCCT | |
| | ACATGAGCAGAGAGCTGAGCGCCTATCTGGACGACTGCCTGGAATTT | |
| | CCTGAGGGCCGTACAAAGGTGCCCGTGCAGCCTACTAACGGACGGG | |
| | CTACCGCCTGGCTCCGGCACGCCTGGGGCCTGAACTTCGGCGCCCA | |
| | GGGCGAGAAGGACAGGGAGGACGACAGACACCACGCGACCGACGC | |
| | TTGTGTGATCGCTGCATGCAACAGATCTATGCTGATCAAGACCGCCC | |
| | AATACAGCAGCGGAAAAAGAAAGATGAGCGAGGAACAGAAAGATGCT | |
| | GAACTGCGGGAGGCCCAGCCTTGGCCCACCTTTTCTAAGGACGTGA | |
| | CCACCCGCCGGGATTGCGTGATCCCTACACGGTTCGTGCCTCATAAG | |
| | GCCGGCGGCGAGATCTTCGAGCAGACCCTGTACGCCTACAAGGGCA | |
| | CCAACGCCCAGGGAAAGCACATCCTGGCCAAAACCGACGCCTCTAC | |
| | CGGCAAAGAGAAAGAGACAGTGGCCGGAAATGCCGTTGTTTCTGTG | |
| | GACGGCAAGTCCGCCGTGATCGTGTCCGAGATGCTGTGTCTGATGCT | |
| | GTGGCACGACGCCGACGCCAGGCCGAACGGTAAGGTGAAGGGACA | |
| | GTGGTATGCCGACCCCATCTATCGGGCCGACCTGCCTGCCCTGAGA | |
| | GATGGGACCTTCGTGCACAGAATCGCCAAAGCCCACAAGGGCCGAG | |
| | ATGTGTGGCCTGAGATTCCAGACAGAGTGCTGGAGAACACCAAGCCT | |
| | ATCGTCGTGTACCGGGGCGACGTGCTGATGGTGGATGATGCCATCG | |
| | GCAGATTCGCCGGCATCGATATCGATGGCGTGAGATGGGAGTTCGC | |
| | CCAGATTAGCCCTGATGTGCCTGCCGAAATCGATACAATCGGTAGCC | |
| | TGGGCAACGAGAACAAGGTGAAGCTCGTGACAGAGGATGTGCTGGG | |
| | ACACTGCTGGGGACAGCTGAACATGAAAGTAGGCTGCGCCGAG | |
| BDKL Type II | ATGATCCCCAATCCCCTGCTGGGCCTGGACAAGCGGACTGCCGACG | 60 |
| Cas | GCAGTGAGTTCGAGTCTCCCAAGAAAAAGAGGAAAGTGggatccCCTAT | |
| mammalian | GGCCGTGAATCGGGAAGTGGGAAAGCTGGGCAATCCTAGATACCTG | |
| expression | GTGCTGGGCCTGGACCCTGGCATCGCCAGCTGCGGCTTCGCCCTGC | |
| construct | TGGATCTGAACAATCACGAAATCCTTGAGATGGGCAGCAGACTGTTC | |
| (includes N- | GACGCACCAACAGTGCCTAAGACCGGCCAGAGCAAGGCCGTGGTGC | |
| terminal SV5 | GGAGAATGCACCGCTCTACAAGAAGAAACCTGGATAGGACACAGGAC | |
| tag and NLS | CGGCTGAAGCACTGTCTGAATGCCCTCAAGGAAGCCGATCTGGTTCC | |
| and C- | AGAGGACGCGACAAAGGAATACTTCCACACCCAAAAGGGCGATAAGC | |
| terminal NLS) | CTCCACTGAGGCTGAGAGTGGACGGCCTGGACCGGCGGCTGACCGA | |
| (nt) | GAGAGAATGGGCCATCGTGCTGTACAGCCTGTGCAAGAGAAGAGGC | |
| | TACATCCCCCACGGCGAAGGCTCTGTGGATGGCAGCAGCGAGGACG | |
| | GCAAGGTCTTGGCCGCCGTGAGCGCCAACGCTAAGCTGCTGGACGA | |
| | GGGAAAATACAGAACCGTGGGAGAATGGCTGGCCAGTCAGGAGAGA | |
| | TCCCGGAACAGAGCCGGCTCCTACGAGAAATGCGTGACACACAAACT | |
| | GCTGATGGAAGAGGCTTCTACTCTTTTTGCCAAGCAAAGAGGCTTCG | |
| | GCAACGAGTATGCCAGCCCTGAATTGGAAAAGAAGTACGTGGGTATT | |
| | TGTGATTGGGAGAAGCCCAGAGATGCATTTGACGAACGCGTCTACCA | |
| | GACACAAGTGGGACATTGCATCTACTTTCACGATGAAAAGAGAGCTG | |
| | CCAGAAGCACGCTGAGCAACGAGCTGGTGTCGGCTTACGCAGCTTT | |
| | CGGCAACATCTCTATCGTGCAGCCTGGCCACGAAACCAGACGGGTC | |
| | AGCCCCGACGTTCGGGACCAGTGCATCGGCCTGCTGTTCAGCGCCT | |
| | CCCCAGACGGTCCTGCCAGCGGCACAAAGGTGACCTACAAAAGCCT | |
| | GAGAAAGCTGCTGGACCTGTCTGCTAAGGCCTCTTTTAAGGGCATCA | |
| | ACGTGGACGACGAGAAGAGCCGGGAAGTTTTCCAACCCAAGGGCTG | |
| | GAGAGCTCTGAGGGCCACCCTGGTGGACAGCGCCCCTGAGCTGCTG | |
| | ATGAGACTCAGAACAGACATCGACCTGGCCGACGAGGTGTTCGAGG | |
| | CTGCCGCTTACAGCTCTTCTATCGAGGTGCTGAGAAATAGGCTGGGC | |
| | TTGCTGGATCTGAGCGACGAGGAAGTGGACAAGCTGTCCAGACTGC | |
| | CATACGCCAGCAGAGTGCTGAACGGCTACGGCAGCCGGTCCAAGAA | |
| | GGCCCTGGACATCCTGCTGGACCAGTTCGAGGACCCTGAAATCCTG | |
| | GACCTGGCTGAAGCCGAACGGAGCAGCGGCCTGCTCGGCAAGCGG | |
| | ATGGCGGGCAACGGCATCGCTAAAAGCGACAGACTGATGCCTTACAA | |
| | GTGCTGGCTGGAGGACACCGGCAGAACAAACAACAACCCTGTGGTC | |
| | CTGCGGGCCATCGCCCAGATGCGCAAGGTGGTGAACGCCATTTGCA | |
| | GAGAGTGGGGCGTGCCTCATGAGATCCACGTGGAACTGGATAGAGA | |
| | ACTGAAGCTGAGCAAAAAGCAGCAGGACAAGATCCGGAAGAACAACA | |
| | AGAAGAACGAGGCCGAAAACGCCCGGGTGGCCAAGCAGATCGCCG | |
| | GCCTGCTGGGCTGTGACCCCGGCAGCGTTAGCAGAAATCAGATCAG | |
| | AAAGTACACCCTGTGGGAGCAGCAAGATAATGTGGACATCTACACCG | |
| | GAGATGCCATCGATCTGGAACGGCTCCTGGCTGACAGTATGTACACC | |
| | CAGATCGACCACGTGCTCCCCTTCAGCAGAACCGGCGACAACGCCT | |
| | GGTACAACAAAGTGCTCTGCCTGGCCAGCTCCAACCAGCACAAGCG | |
| | GGAGCGGTCACCCTACGAGTGGATGACCAGCGGCGAGGCCGGAGC | |
| | CCCTAGCTGGGAAGAGTTCCAGGCCCGAGTGGTGGAAAACCACAAG | |
| | CTGGGATACAAGAAAAGATCCTTCCTGCTGGAAGAGAATCTGGATGC | |
| | CAAGGTGGACGACTTCCTGAGAAGAAGCATCACCGACACCGCCTACA | |
| | TGAGCAGAGAGCTGAGCGCCTATCTGGACGACTGCCTGGAATTTCCT | |
| | GAGGGCCGTACAAAGGTGCCCGTGCAGCCTACTAACGGACGGGCTA | |
| | CCGCCTGGCTCCGGCACGCCTGGGGCCTGAACTTCGGCGCCCAGG | |
| | GCGAGAAGGACAGGGAGGACGACAGACACCACGCGACCGACGCTT | |
| | GTGTGATCGCTGCATGCAACAGATCTATGCTGATCAAGACCGCCCAA | |
| | TACAGCAGCGGAAAAAGAAAGATGAGCGAGGAACAGAAAGATGCTGA | |
| | ACTGCGGGAGGCCCAGCCTTGGCCCACCTTTTCTAAGGACGTGACC | |
| | ACCCGCCGGGATTGCGTGATCCCTACACGGTTCGTGCCTCATAAGGC | |
| | CGGCGGCGAGATCTTCGAGCAGACCCTGTACGCCTACAAGGGCACC | |
| | AACGCCCAGGGAAAGCACATCCTGGCCAAAACCGACGCCTCTACCG | |
| | GCAAAGAGAAAGAGACAGTGGCCGGAAATGCCGTTGTTTCTGTGGAC | |
| | GGCAAGTCCGCCGTGATCGTGTCCGAGATGCTGTGTCTGATGCTGTG | |
| | GCACGACGCCGACGCCAGGCCGAACGGTAAGGTGAAGGGACAGTG | |
| | GTATGCCGACCCCATCTATCGGGCCGACCTGCCTGCCCTGAGAGAT | |
| | GGGACCTTCGTGCACAGAATCGCCAAAGCCCACAAGGGCCGAGATG | |
| | TGTGGCCTGAGATTCCAGACAGAGTGCTGGAGAACACCAAGCCTATC | |
| | GTCGTGTACCGGGGCGACGTGCTGATGGTGGATGATGCCATCGGCA | |
| | GATTCGCCGGCATCGATATCGATGGCGTGAGATGGGAGTTCGCCCA | |
| | GATTAGCCCTGATGTGCCTGCCGAAATCGATACAATCGGTAGCCTGG | |
| | GCAACGAGAACAAGGTGAAGCTCGTGACAGAGGATGTGCTGGGACA | |
| | CTGCTGGGGACAGCTGAACATGAAAGTAGGCTGCGCCGAGtctagaAA | |
| | GCGGACAGCAGACGGCTCCGAATTTGAAAGCCCTAAGAAAAAGAGAA | |
| | AGGTGTGA | |

In some embodiments an BDKL Type II Cas protein comprises an amino acid sequence of SEQ ID NO:55, SEQ ID NO:56, or SEQ ID:57. In some embodiments, a BDKL Type II Cas protein has nickase activity, for example resulting from one or more amino acid substitutions relative to the sequence of SEQ ID NO:55, SEQ ID NO:56, or SEQ ID:57. In some embodiments, the one or more amino acid substitutions providing nickase activity is a D23A substitution, wherein the position of the D23A substitution is defined with respect to the amino acid numbering of SEQ ID NO:56. In some embodiments, the one or more amino acid substitutions providing nickase activity is a N632A substitution, wherein the position of the N632A substitution is defined with respect to the amino acid numbering of SEQ ID NO:56. In some embodiments, a BDKL Type II Cas protein is catalytically inactive, for example due to both a D23A substitution and a N632A substitution, where the positions of the substitutions are defined with respect to the amino acid numbering of SEQ ID NO:56.

### 6.2.3. Fusion and Chimeric Proteins

The disclosure provides Type II Cas proteins (e.g., an AHZW Type II Cas protein as described in Section 6.2.1.1, an ABSE Type II Cas protein as described in Section 6.2.1.2, an AIXM Type II Cas protein as described in Section 6.2.1.3, an AXTQ Type II Cas protein as described in Section 6.2.1.4, an AIWM Type II Cas protein as described in Section 6.2.1.5, an AIWR Type II Cas protein as described in Section 6.2.1.6, an AIYQ Type II Cas protein as described in Section 6.2.1.7, an EQSC Type II Cas protein as described in Section 6.2.2.1, a BDLP Type II Cas protein as described in Section 6.2.2.2, or a BDKL Type II Cas protein as described in Section 6.2.2.3) which are in the form of fusion proteins comprising a Type II Cas protein sequence fused with one or more additional amino acid sequences, such as one or more nuclear localization signals and/or one or more non-native tags. Fusion proteins can also comprise an amino acid sequence of, for example, a nucleoside deaminase, a reverse transcriptase, a transcriptional activator (e.g., VP64), a transcriptional repressor (e.g., Krüppel associated box (KRAB)), a histone-modifying protein, an integrase, or a recombinase.

In some embodiments, a fusion protein of the disclosure comprises a means for localizing the Type II Cas protein to the nucleus, for example a nuclear localization signal.

Non-limiting examples of nuclear localization signals include KRTADGSEFESPKKKRKV (SEQ ID NO:109), PKKKRKV (SEQ ID NO:110), PKKKRRV (SEQ ID NO:111), KRPAATKKAGQAKKKK (SEQ ID NO:112), YGRKKRRQRRR (SEQ ID NO:113), RKKRRQRRR (SEQ ID NO:114), PAAKRVKLD (SEQ ID NO:115), RQRRNELKRSP (SEQ ID NO:116), VSRKRPRP (SEQ ID NO:117), PPKKARED (SEQ ID NO:118), PQPKKKPL (SEQ ID NO:119), SALIKKKKKMAP (SEQ ID NO:120), PKQKKRK (SEQ ID NO:121), RKLKKKIKKL (SEQ ID NO:122), REKKKFLKRR (SEQ ID NO:123), KRKGDEVDGVDEVAKKKSKK (SEQ ID NO:124), RKCLQAGMNLEARKTKK (SEQ ID NO:125), NQSSNFGPMKGGNFGGRSSGPYGGGGQYFAKPRNQGGY (SEQ ID NO:126), and RMRIZFKNKGKDTAELRRRRVEVSVELRKAKKDEQILKRRNV (SEQ ID NO:127).

Exemplary fusion partners include protein tags (e.g., V5-tag (e.g., having the sequence GKPIPNPLLGLDST (SEQ ID NO:128) or IPNPLLGLD (SEQ ID NO:129)), FLAG-tag, myc-tag, HA-tag, GST-tag, polyHis-tag, MBP-tag), protein domains, transcription modulators, enzymes acting on small molecule substrates, DNA, RNA and protein modification enzymes (e.g., adenosine deaminase, cytidine deaminase, guanosyl transferase, DNA methyltransferase, RNA methyltransferases, DNA demethylases, RNA demethylases, dioxygenases, polyadenylate polymerases, pseudouridine synthases, acetyltransferases, deacetylase, ubiquitin-ligases, deubiquitinases, kinases, phosphatases, NEDD8-ligases, de-NEDDylases, SUMO-ligases, deSUMOylases, histone deacetylases, reverse transcriptases, histone acetyltransferases histone methyltransferases, histone demethylases), protein DNA binding domains, RNA binding proteins, polypeptide sequences with specific biological functions (e.g., nuclear localization signals, mitochondrial localization signals, plastid localization signals, subcellular localization signals, destabilizing signals, Geminin destruction box motifs), and biological tethering domains (e.g., MS2, Csy4 and lambda N protein). Various Type II Cas fusion proteins are described in Ribeiro et al., 2018, In. J. Genomics, Article ID:1652567; Jayavaradhan, et al., 2019, Nat Commun 10:2866; Xiao et al., 2019, The CRISPR Journal, 2(1):51-63; Mali et al., 2013, Nat Methods. 10(10):957-63; U.S. Pat. Nos. 9,322,037, and 9,388,430. In some embodiments, a fusion partner is an adenosine deaminase. An exemplary adenosine deaminase is the tRNA adenosine deaminase (TadA) moiety contained in the adenine base editor ABE8e (Richter, 2020, Nature Biotechnology 38:883-891). The TadA moiety of ABE8e comprises the following amino acid sequence:

(SEQ ID NO: 130)
SEVEFSHEYWMRHALTLAKRARDEREVPVGAVLVLNNRVIGEGWNRAIGL
HDPTAHAEIMALRQGGLVMQNYRLIDATLYVTFEPCVMCAGAMIHSRIGR
VVFGVRNSKRGAAGSLMNVLNYPGMNHRVEITEGILADECAALLCDFYRM
PRQVFNAQKKAQSSIN

In some embodiments, an adenosine deaminase fusion partner comprises an amino acid sequence having at least 50%, at least 60%, at least 70%, at least 80%, at least 90%, at least 95%, at least 97%, at least 98%, at least 99% amino acid sequence identity with SEQ ID NO:130.

Type II Cas proteins of the disclosure in the form of a fusion protein comprising an adenosine deaminase can be used as an adenine base editor to change an “A” to a “G” in DNA. Type II Cas proteins of the disclosure in the form of a fusion protein comprising a cytidine deaminase can be used as a cytosine base editor to change a “C” to a “T” in DNA.

In some embodiments, a fusion protein of the disclosure comprises a means for deaminating adenosine, for example an adenosine deaminase, e.g., a TadA variant. In some embodiments, a fusion protein of the disclosure comprises a means for deaminating cytidine, for example a cytidine deaminase, e.g., cytidine deaminase 1 (CDA1) or an apolipoprotein B mRNA-editing complex (APOBEC) family deaminase (Cheng et al., 2019, Nat Commun. 10(1):3612; Gehrke et al., 2018, Nat Biotechnol. 36(10):977-982).

In some embodiments, a fusion protein of the disclosure comprises a means for synthesizing DNA from a single-stranded template, for example a reverse transcriptase. Type II Cas proteins of the disclosure in the form of a fusion protein comprising a reverse transcriptase (RT) can be used as a prime editor to carry out precise base editing without double-stranded DNA breaks.

In some embodiments, a fusion protein of the disclosure is a prime editor, e.g., a Type II Cas protein fused to a suitable RT (e.g., Moloney murine leukemia virus (M-MLV) RT or other RT enzyme). Such fusion proteins can be used in conjunction with a prime editing guide RNA (pegRNA) that both specifies the target site and encodes the desired edit (Anzalone et al., 2019, Nature, 576(7785):149-157).

In some embodiments, a fusion protein of the disclosure comprises one or more nuclear localization signals positioned N-terminal and/or C-terminal to a Type II Cas protein sequence (e.g., a AHZW Type II Cas protein having a sequence of SEQ ID NO:1)). In some embodiments, a fusion protein of the disclosure comprises an N-terminal and a C-terminal nuclear localization signal, for example each having the sequence KRTADGSEFESPKKKRKV (SEQ ID NO:109).

The disclosure provides chimeric Type II Cas proteins comprising one or more domains of an AHZW Type II Cas protein and one or more domains of one or more different proteins (e.g., one or more different Type II Cas proteins), chimeric Type II Cas proteins comprising one or more domains of an ABSE Type II Cas protein and one or more domains of one or more different proteins (e.g., one or more different Type II Cas proteins), chimeric Type II Cas proteins comprising one or more domains of an AIXM Type II Cas protein and one or more domains of one or more different proteins (e.g., one or more different Type II Cas proteins), chimeric Type II Cas proteins comprising one or more domains of an AXTQ Type II Cas protein and one or more domains of one or more different proteins (e.g., one or more different Type II Cas proteins), chimeric Type II Cas proteins comprising one or more domains of an AIWR Type II Cas protein and one or more domains of one or more different proteins (e.g., one or more different Type II Cas proteins), chimeric Type II Cas proteins comprising one or more domains of an AIWM Type II Cas protein and one or more domains of one or more different proteins (e.g., one or more different Type II Cas proteins), chimeric Type II Cas proteins comprising one or more domains of an AIYQ Type II Cas protein and one or more domains of one or more different proteins (e.g., one or more different Type II Cas proteins), chimeric Type II Cas proteins comprising one or more domains of an EQSC Type II Cas protein and one or more domains of one or more different proteins (e.g., one or more different Type II Cas proteins), chimeric Type II Cas proteins comprising one or more domains of a BDLP Type II Cas protein and one or more domains of one or more different proteins (e.g., one or more different Type II Cas proteins), and chimeric Type II Cas proteins comprising one or more domains of a BDKL Type II Cas protein and one or more domains of one or more different proteins (e.g., one or more different Type II Cas proteins).

The domain structures of wild-type AIK, BNK, HPLH, and ANAB Type II Cas proteins were inferred by multiple alignment with the amino acid sequences of Type II Cas proteins for which the crystal structure is known and for which it is thus possible to define the boundaries of each functional domain. The domains identified in Type II Cas proteins are: the RuvC catalytic domain (discontinuous, represented by RuvC-I, RuvC-II, and RuvC-III domains), bridge helix (BH), recognition (REC) domain, HNH catalytic domain, wedge (WED) domain, and PAM-interacting domain (PID).

Tables 3A-3B below report the amino acid positions corresponding to the boundaries between different functional domains in wild-type AHZW (SEQ ID NO:2), ABSE (SEQ ID NO:8), AIXM (SEQ ID NO:14), AXTQ (SEQ ID NO:20), AIWM (SEQ ID NO:26), AIWR (SEQ ID NO:32), AIYQ (SEQ ID NO:38), EQSC (SEQ ID NO:44), BDLP (SEQ ID NO:50), and BDKL (SEQ ID NO:56) Type II Cas proteins.

TABLE 3A
Amino Acid Positions of AHZW, ABSE,
AIXM, AXTQ, AIWM, AIWR, AIYQ Domains

| Domain | AHZW | ABSE | AIXM | AXTQ | AIWM | AIWR | AIYQ |
|---|---|---|---|---|---|---|---|
| RuvC-I | 1-46 | 1-41 | 1-42 | 1-38 | 1-42 | 1-42 | 1-42 |
| BH | 47-79 | 42-74 | 43-75 | 39-71 | 43-75 | 43-75 | 43-75 |
| REC | 80-430 | 75-429 | 76-431 | 72-444 | 76-431 | 76-431 | 76-431 |
| RuvC-II | 431-488 | 430-485 | 432-489 | 445-504 | 432-489 | 432-489 | 432-489 |
| HNH | 489-657 | 486-654 | 490-656 | 505-676 | 490-656 | 490-656 | 490-656 |
| RuvC-III | 658-797 | 655-791 | 657-797 | 677-819 | 657-797 | 657-797 | 657-797 |
| WED | 798-833 | 792-827 | 798-838 | 820-855 | 798-838 | 798-838 | 798-838 |
| PID | 834-1073 | 828-1057 | 839-1071 | 856-1096 | 839-1071 | 839-1071 | 839-1069 |

TABLE 3B
Amino Acid Positions of EQSC, BDLP, and BDKL Domains

| Domain | EQSC | BDLP | BDKL |
|---|---|---|---|
| RuvC-I | 1-61 | 1-61 | 1-61 |
| BH | 62-94 | 62-94 | 62-94 |
| REC | 95-466 | 95-466 | 95-469 |
| RuvC-II | 467-525 | 467-525 | 470-528 |
| HNH | 526-689 | 526-689 | 529-692 |
| RuvC-III | 690-835 | 690-835 | 693-829 |
| WED | 836-877 | 836-877 | 830-875 |
| PID | 878-1006 | 878-1008 | 876-1019 |

A chimeric Type II Cas protein can comprise one of more of the following domains (e.g., one or more, two or more, three or more, four or more, five or more, six or more, seven or more) from an AHZW Type II Cas protein, ABSE Type II Cas protein, AIXM Type II Cas proteins, and AXTQ Type II Cas protein, AIWM Type II Cas protein, AIWR Type II protein, AIYQ Type II protein, EQSC Type II protein, BDLP Type II protein, and/or BDKL Type II protein, and one or more domains from one or more other proteins, for example SaCas9, SpCas9 or a Type II Cas protein described in US 2020/0332273, US 2019/0169648, or 2015/0247150 (the contents of each of which are incorporated herein by reference in their entirety): RuvC-I, BH, REC, RuvC-II, HNH, RuvC-III, WED, PID. For example, the PID domain can be swapped between different Type II Cas proteins to change the PAM specificity of the resulting chimeric protein (which is given by the donor PID domain). Swapping of other domains or portions of them is also within the scope of the disclosure (e.g., through protein shuffling).

In some embodiments, a Type II Cas protein of the disclosure comprises one, two, three, four, five, six, seven, or eight of a RuvC-I domain, a BH domain, a REC domain, a RuvC-II domain, a HNH domain, a RuvC-III domain, a WED domain, and a PID domain arranged in the N-terminal to C-terminal direction. In some embodiments, all domains are from an AHZW Type II Cas protein (e.g., an AHZW Type II Cas protein whose amino acid sequence comprises SEQ ID NO:1, 2, or 3). In some embodiments, all domains are from an ABSE Type II Cas protein (e.g., an ABSE Type II Cas protein whose amino acid sequence comprises SEQ ID NO:7, 8, or 9). In some embodiments, all domains are from an AIXM Type II Cas protein (e.g., an AIXM Type II Cas protein whose amino acid sequence comprises SEQ ID NO:13, 14, or 15). In some embodiments, all domains are from an AXTQ Type II Cas protein (e.g., an AXTQ Type II Cas protein whose amino acid sequence comprises SEQ ID NO:19, 20, or 21). In some embodiments, all domains are from an AIWM Type II Cas protein (e.g., an AIWM Type II Cas protein whose amino acid sequence comprises SEQ ID NO:25, 26, or 27). In some embodiments, all domains are from an AIWR Type II Cas protein (e.g., an AIWR Type II Cas protein whose amino acid sequence comprises SEQ ID NO:31, 32, or 33). In some embodiments, all domains are from an AIYQ Type II Cas protein (e.g., an AIYQ Type II Cas protein whose amino acid sequence comprises SEQ ID NO:37, 38, or 39). In some embodiments, all domains are from an EQSC Type II Cas protein (e.g., an EQSC Type II Cas protein whose amino acid sequence comprises SEQ ID NO:43, 44, or 45). In some embodiments, all domains are from a BDLP Type II Cas protein (e.g., a BCLP Type II Cas protein whose amino acid sequence comprises SEQ ID NO:49, 50, or 51). In some embodiments, all domains are from a BDKL Type II Cas protein (e.g., a BDKL Type II Cas protein whose amino acid sequence comprises SEQ ID NO:55, 56, or 57). In other embodiments, one or more domains (e.g., one domain), e.g., a PID domain, is from another Type II Cas protein.

In addition, one or more amino acid substitutions can be introduced in one or more domains to modify the properties of the resulting nuclease in terms of editing activity, targeting specificity or PAM recognition specificity. For example, one or more amino acid substitutions can be introduced to provide nickase activity. Exemplary amino acid substitutions in SaCas9 providing nickase activity are the D10A substitution in the RuvC domain and the N580A substitution in the HNH domain. Combining both the D10A and N580A substitutions in SaCas9 provides a catalytically inactive nuclease. Corresponding substitutions can be introduced into the Type II Cas nucleases of the disclosure to provide nickases and catalytically inactive Cas proteins. For example, an AHZW Type II Cas protein can include a D13A substitution (corresponding to D10A in SaCas9) or a N589A substitution (corresponding to N580A in SaCas9) to provide a nickase, or D13A and N589A substitutions to provide a catalytically inactive Cas protein, where the positions of the D13A and N589A substitutions are defined with respect to amino acid numbering of SEQ ID NO:2. Positions corresponding to D10 and N580 of SaCas9 for Type II Cas proteins of the disclosure as shown in Table 4. Nickases and catalytically inactive Type II Cas proteins of the disclosure can be used, for example, in base editors comprising a cytosine or adenosine deaminase fusion partner. Catalytically inactive Type II Cas proteins can also be used, for example, as fusion partners for transcriptional activators or repressors.

TABLE 4

| | | | Reference |
|---|---|---|---|
| II | Position | Position | SEQ ID NO |
| Type | corresponding | corresponding | defining |
| Cas | to D10 | to N580 | amino acid |
| Protein | of SaCas9 | of SaCas9 | numbering |
| AHZW | D13 | N589 | 2 |
| ABSE | D8 | N587 | 8 |
| AIXM | D9 | N590 | 14 |
| AXTQ | D6 | N611 | 20 |
| AIWM | D9 | N590 | 26 |
| AIWR | D9 | N590 | 32 |
| AIYQ | D9 | N590 | 38 |
| EQSC | D23 | N629 | 44 |
| BDLP | D23 | N629 | 50 |
| BDKL | D23 | N632 | 56 |

### 6.3. Guide RNAs

The disclosure provides gRNA molecules that can be used with Type II Cas proteins of the disclosure to edit genomic DNA, for example mammalian DNA, e.g., human DNA. gRNAs of the disclosure typically comprise a spacer of 15 to 30 nucleotides in length. The spacer can be positioned 5′ of a crRNA scaffold to form a full crRNA. The crRNA can be used with a tracrRNA to effect cleavage of a target genomic sequence.

An exemplary crRNA scaffold sequence that can be used for AHZW Type II Cas gRNAs comprises GUUCUGCUACCAUCGAAAUUUUUGCUAGGCUACAAC (SEQ ID NO:61) and an exemplary tracrRNA sequence that can be used for AHZW Type II Cas gRNAs comprises

(SEQ ID NO: 62)
UUGUAGUCUAGCAAAGGUUUUGAUGAUCUAGCAGAACAAGGGUUUAUCCC
GGAAUCGACUCCUUAGGGAGUCUUUUUU.

An exemplary crRNA scaffold sequence that can be used for ABSE Type II Cas gRNAs comprises GUUUUGGUACCCUCUAAAUUUUUGCUAUACUGAAA (SEQ ID NO:63) and an exemplary tracrRNA sequence that can be used for ABSE Type II Cas gRNAs comprises

(SEQ ID NO: 64)
CAGUAUAGCAAAGGUUUAGAGGACCUAUCAAAACAAGGGAAUUAUUCCCG
AAAUCGGAACUGCUAAGCAGUUCCUUUUUU.

An exemplary crRNA scaffold sequence that can be used for AIXM Type II Cas gRNAs comprises GUUUUGCUACCCUCACAAUUUUUGCUAUGUAAAGAC (SEQ ID NO:65) and an exemplary tracrRNA sequence that can be used for AIXM Type II Cas gRNAs comprises

(SEQ ID NO: 66)
UUACAUAGCAAAGAUUGUGAGGAUCUAGCGAAACAAGGGCUGUCUUAGGA
CAUGUCCCGAAAUCGAGUCUCGUAGGAGACUCUUUUUU.

An exemplary crRNA scaffold sequence that can be used for AXTQ Type II Cas gRNAs comprises GUUUUAGUACCUGAAAGAAUUGAGUUAUUGUAAAAC (SEQ ID NO:67) and an exemplary tracrRNA sequence that can be used for AXTQ Type II Cas gRNAs comprises

(SEQ ID NO: 68)
GUUUUGCAAUAACUCAAUUUUUUCAGAUCUACUAAAACAAGGCUUUAUGC
CGAAAUCAAGGACACAGAUAAGUGUCCUUUUUU.

An exemplary crRNA scaffold sequence that can be used for AIWM Type II Cas gRNAs, AIWR Type II Cas gRNAs, and AIYQ Type II Cas gRNAs comprises GUUUUGCUACCCUCACAAUUUUUGCUAUGUAAAGAC (SEQ ID NO:69) and an exemplary tracrRNA sequence that can be used for AIWM Type II Cas gRNAs, AIWR Type II Cas gRNAs, and AIYQ Type II Cas gRNAs comprises

(SEQ ID NO: 70)
UUACAUAGCAAAGAUUGUGAGGAUCUAGCAAAACAAGGGCUGUCUUAGGA
CAUGUCCCGAAAUCGAGUCUCGUAGGAGACUCUUUUUU.

An exemplary crRNA scaffold sequence that can be used for EQSC Type II Cas gRNAs comprises GUCUUGAGCUCGCACUUUUCCCCAAGCUGAUACAAU (SEQ ID NO:71) and an exemplary tracrRNA sequence that can be used for EQSC Type II Cas gRNAs comprises

(SEQ ID NO: 72)
UCACCUUGGGGAAAAGUGCGAGACUCCAGACAAGGGGAACCUACAACGGU
AGGUUCACCCGUAGGGUUACCCCCGCGUCAUCUUCGGAAGGCGCGGGGCG
AACUCUUUUUU.

An exemplary crRNA scaffold sequence that can be used for BDLP Type II Cas gRNAs comprises GUCUUGAGCUCGCACUUUUCCCCAAGCUGAUACAAU (SEQ ID NO:73) and an exemplary tracrRNA sequence that can be used for BDLP Type II Cas gRNAs comprises

(SEQ ID NO: 74)
UCACCUUGGGGAAAAGUGCGAGACUCCAGACAAGGGGAGUCUACAACAGU
AGGUUCACCCGUAGGGUUACCCCCGCGUCAUCCUCGGAAGGCGCGGGGCG
AACUCUUUUUU.

An exemplary crRNA scaffold sequence that can be used for BDKL Type II Cas gRNAs comprises GUCUUGAGUUUGCGCCCUUCCCCAAGGUGAUACGCU (SEQ ID NO:75) and an exemplary tracrRNA sequence that can be used for BDKL Type II Cas gRNAs comprises

(SEQ ID NO: 76)
UCACCUUGGGGAAGGGCGCUGCUCCAGACAAGGGAAGCCACUUGCUGGCU
UACCCGUAAAGUUUCAACCCCGCGUUGCCUUCAGGCGGCGCGGGGUGAAC
UUUUUU.

gRNAs of the disclosure are in some embodiments single guide RNAs (sgRNAs), which typically comprise the spacer at the 5′ end of the molecule and a 3′ sgRNA scaffold. Alternatively, gRNAs can comprise separate crRNA and tracrRNA molecules.

Further features of exemplary gRNA spacer sequences are described in Section 6.3.1 and further features of exemplary 3′ sgRNA scaffolds are described in Section 0.

### 6.3.1. Spacers

The spacer sequence is partially or fully complementary to a target sequence found in a genomic DNA sequence, for example a human genomic DNA sequence. For example, a spacer sequence can be partially or fully complementary to a nucleotide sequence in a gene having a disease causing mutation. A spacer that is partially complementary to a target sequence can have, for example, one, two, or three mismatches with the target sequence.

gRNAs of the disclosure can comprise a spacer that is 15 to 30 nucleotides in length (e.g., 15 to 25, 16 to 24, 17 to 23, 18 to 22, 19 to 21, 18 to 30, 20 to 28, 22 to 26, or 23 to 25 nucleotides in length). In some embodiments, a spacer is 15 nucleotides in length. In other embodiments, a spacer is 16 nucleotides in length. In other embodiments, a spacer is 17 nucleotides in length. In other embodiments, a spacer is 18 nucleotides in length. In other embodiments, a spacer is 19 nucleotides in length. In other embodiments, a spacer is 20 nucleotides in length. In other embodiments, a spacer is 21 nucleotides in length. In other embodiments, a spacer is 22 nucleotides in length. In other embodiments, a spacer is 23 nucleotides in length. In other embodiments, a spacer is 24 nucleotides in length. In other embodiments, a spacer is 25 nucleotides in length. In other embodiments, a spacer is 26 nucleotides in length. In other embodiments, a spacer is 27 nucleotides in length. In other embodiments, a spacer is 28 nucleotides in length. In other embodiments, a spacer is 29 nucleotides in length. In other embodiments, a spacer is 30 nucleotides in length.

Type II Cas endonucleases require a specific sequence, called a protospacer adjacent motif (PAM) that is downstream (e.g., directly downstream) of the target sequence on the non-target strand. Thus, spacer sequences for targeting a gene of interest can be identified by scanning the gene for PAM sequences recognized by the Type II Cas protein. Exemplary PAM sequences for Type II Cas proteins are shown in Table 5A and Table 5B.

TABLE 5A
Exemplary Type II Cas Protein PAM Sequences
(predicted)

| Cas Protein | Sequence |
|---|---|
| AHZW Type II Cas Protein | NNAAG |
| ABSE Type II Cas Protein | NNAA |
| AIXM Type II Cas Protein | NNAGG |
| AXTQ Type II Cas Protein | NNG |
| AIWM Type II Cas Protein | NNGA |
| AIWR Type II Cas Protein | NNAAA |
| AIYQ Type II Cas Protein | NNGGNNAA |

N = A, T, C, or G

TABLE 5B
Exemplary Type II Cas Protein PAM Sequences (in vitro determined)

| Type II Cas protein | in vitro-determined PAM | Most preferred PAMs |
|---|---|---|
| AHZW Type II Cas | NYRRR, NNRRR | NYARR, NYAAR, NYARRNY, NYAARNY |
| ABSE Type II Cas | NYRRR, NNARR | NYAAR, NYARR |
| AIXM Type II Cas | NNRGR | NYRGR, NYAGG, NYRGG, NNAGG |
| AXTQ Type II Cas | NNGNNNYN, NNGNNNYR | NNGYNNYR, NNGYNNCR, NNGNVHYR, |
| | | NNGYVHYR, NNGYVHCR, NNGHNHYR |
| AIWM Type II Cas | NNGRR | NYGRR |
| AIWR Type II Cas | NYRRR, NNARR | NYRRRNY, NYARGNY, NYARRNY |
| AIYQ Type II Cas | NNGG | NNGGWW, NNGGAW |
| EQSC Type II Cas | NNNNCNNA | NNNNCKNA |
| BDLP Type II Cas | NNNNGT | |
| BDKL Type II Cas | NNNNCNNA, NNNNCC | NNNNCMNA, NNNNCCNA |

N = A, T, C, or G; H = A, C, or T; V = A, C, or G; R = G or A; Y = C or T; W = A or T; K = G or T

Example 3 describes exemplary sequences that can be used to target RHO genomic sequences. Example 4 describes exemplary sequences that can be used to target TRAC, B2M, and PD1. In some embodiments, a gRNA of the disclosure comprises a spacer sequence targeting RHO. In some embodiments, a gRNA of the disclosure comprises a spacer sequence targeting TRAC. In some embodiments, a gRNA of the disclosure comprises a spacer sequence targeting B2M. In some embodiments, a gRNA of the disclosure comprises a spacer sequence targeting PD1.

Additional exemplary spacer sequences that can be used in gRNAs of the disclosure are set forth in Table 6.

TABLE 6
Exemplary spacer sequences

| Type II Cas | Target | Name | Spacer Sequence | SEQ ID NO: |
|---|---|---|---|---|
| EQSC | RHO | gRNA-EQSC-RHOrs7984A/G-g1 | UUGUGGCUGACCCGYGGCUGCUC | 287 |
| EQSC | RHO | gRNA-EQSC-RHOrs7984A-g1 | UUGUGGCUGACCCGUGGCUGCUC | 288 |
| EQSC | RHO | gRNA-EQSC-RHOrs7984G-g1 | UUGUGGCUGACCCGCGGCUGCUC | 289 |
| EQSC | RHO | gRNA-EQSC-RHOrs7984A/G-g2 | CUUGUGGCUGACCCGYGGCUGCU | 290 |
| EQSC | RHO | gRNA-EQSC-RHOrs7984A-g2 | CUUGUGGCUGACCCGUGGCUGCU | 291 |
| EQSC | RHO | gRNA-EQSC-RHOrs7984G-g2 | CUUGUGGCUGACCCGCGGCUGCU | 292 |
| EQSC | RHO | gRNA-EQSC-RHOrs7984A/G-g3 | GGCCCUUGUGGCUGACCCGYGGC | 293 |
| EQSC | RHO | gRNA-EQSC-RHOrs7984A-g3 | GGCCCUUGUGGCUGACCCGUGGC | 294 |
| EQSC | RHO | gRNA-EQSC-RHOrs7984G-g3 | GGCCCUUGUGGCUGACCCGCGGC | 295 |
| EQSC | RHO | gRNA-EQSC-RHOrs7984A/G-g4 | CUUGGGUGGGAGCAGCCRCGGGU | 296 |
| EQSC | RHO | gRNA-EQSC-RHOrs7984A-g4 | CUUGGGUGGGAGCAGCCACGGGU | 297 |
| EQSC | RHO | gRNA-EQSC-RHOrs7984G-g4 | CUUGGGUGGGAGCAGCCGCGGGU | 298 |
| EQSC | RHO | gRNA-EQSC-RHOrs7984A/G-g5 | GAGCAGCCRCGGGUCAGCCACAA | 299 |
| EQSC | RHO | gRNA-EQSC-RHOrs7984A-g5 | GAGCAGCCACGGGUCAGCCACAA | 300 |
| EQSC | RHO | gRNA-EQSC-RHOrs7984G-g5 | GAGCAGCCGCGGGUCAGCCACAA | 301 |
| BDLP | RHO | gRNA-BDLP-RHOrs7984A-g1 | CAUGGCUGUGGCCCUUGUGGCUG | 302 |
| AHZW | RHO | gRNA-AHZW-rs7984A/G-g1 | GUGGGAGCAGCCRCGGGUCAGCC | 303 |
| AHZW | RHO | gRNA-AHZW-rs7984A-g1 | GUGGGAGCAGCCACGGGUCAGCC | 304 |
| AHZW | RHO | gRNA-AHZW-rs7984G-g1 | GUGGGAGCAGCCGCGGGUCAGCC | 305 |
| AHZW | RHO | gRNA-AHZW-rs7984A/G-g2 | GGCUGACCCGYGGCUGCUCCCAC | 306 |
| AHZW | RHO | gRNA-AHZW-rs7984A-g2 | GGCUGACCCGUGGCUGCUCCCAC | 307 |
| AHZW | RHO | gRNA-AHZW-rs7984G-g2 | GGCUGACCCGCGGCUGCUCCCAC | 308 |
| EQSC | B2M | gRNA-EQSC-B2M-g1 | CAGAAAGAGAGAGUAGCGCGAGC | 309 |
| EQSC | B2M | gRNA-EQSC-B2M-g2 | CACGUCAUCCAGCAGAGAAUGGA | 310 |
| EQSC | B2M | gRNA-EQSC-B2M-g3 | CAUUCUUCAGUAAGUCAACUUCA | 311 |
| BDLP | B2M | gRNA-BDLP-B2M-g1 | AGCAUUCGGGCCGAGAUGUCUCG | 312 |
| BDLP | B2M | gRNA-BDLP-B2M-g2 | GAGAUGUCUCGCUCCGUGGCCUU | 313 |
| BDLP | B2M | gRNA-BDLP-B2M-g3 | GGAUAGCCUCCAGGCCAGAAAGA | 314 |
| BDLP | B2M | gRNA-BDLP-B2M-g4 | UUGACUUUCCAUUCUCUGCUGGA | 315 |
| BDLP | B2M | gRNA-BDLP-B2M-g5 | GGAAAGUCAAAUUUCCUGAAUUG | 316 |
| EQSC | TRAC | gRNA-EQSC-TRAC-g1 | UUUGUCUGUGAUAUACACAUCAG | 317 |
| EQSC | TRAC | gRNA-EQSC-TRAC-g2 | GUAAGGAUUCUGAUGUGUAUAUCA | 318 |
| EQSC | TRAC | gRNA-EQSC-TRAC-g3 | GGCCACAGCACUGUUGCUCUUGA | 319 |
| EQSC | TRAC | gRNA-EQSC-TRAC-g4 | GAGCAACAGUGCUGUGGCCUGGA | 320 |
| EQSC | TRAC | gRNA-EQSC-TRAC-g5 | GGAGCAACAAAUCUGACUUUGCA | 321 |
| BDLP | TRAC | gRNA-BDLP-TRAC-g1 | AAUGUGUCACAAAGUAAGGAUUC | 322 |
| BDLP | TRAC | gRNA-BDLP-TRAC-g2 | UCUGAUGUGUAUAUCACAGACAA | 323 |
| BDLP | TRAC | gRNA-BDLP-TRAC-g3 | GCCUGGAGCAACAAAUCUGACUUU | 324 |
| BDLP | TRAC | gRNA-BDLP-TRAC-g4 | GGCGUUUGCACAUGCAAAGUCAG | 325 |
| BDLP | TRAC | gRNA-BDLP-TRAC-g5 | GAAGAAGGUGUCUUCUGGAAUAA | 326 |
| BDLP | TRAC | gRNA-BDLP-TRAC-g6 | GCUGCCCUUACCUGGGCUGGGGAA | 327 |
| BDLP | PD1 | gRNA-BDLP-PD1-g1 | UGGGCUGGCGGCCAGGAUGGUUC | 328 |
| BDLP | PD1 | gRNA-BDLP-PD1-g2 | CCGCCCAGACGACUGGCCAGGGC | 329 |
| BDLP | PD1 | gRNA-BDLP-PD1-g3 | CAGGCGCCCUGGCCAGUCGUCUG | 330 |
| BDLP | PD1 | gRNA-BDLP-PD1-g4 | AGAACCAUCCUGGCCGCCAGCCC | 331 |
| BDLP | PD1 | gRNA-BDLP-PD1-g5 | CUAAGAACCAUCCUGGCCGCCAG | 332 |
| BDLP | PD1 | gRNA-BDLP-PD1-g6 | GCUGGCGGCCAGGAUGGUUCUUAG | 333 |
| EQSC | PD1 | gRNA-EQSC-PD1-g1 | ACAACUGGGCUGGCGGCCAGGAU | 334 |
| EQSC | PD1 | gRNA-EQSC-PD1-g2 | GUGGGGCUGCUCCAGGCAUGCAG | 335 |
| EQSC | PD1 | gRNA-EQSC-PD1-g3 | GGCCGCCAGCCCAGUUGUAGCAC | 336 |

Position of RHO rs7984 SNP shown in bold (gRNA-BDLP-RHOrs7984A-g1 sequence does not span rs7984 SNP; BDLP PAM is on the SNP); Y = U or C; R = A or G

The RHO spacer sequences in Table 6 are useful for targeting a RHO gene in the vicinity of the rs7984 SNP, located in the 5′ untranslated region (UTR) of the RHO gene. Allele specific targeting can be achieved by using a gRNA targeting the SNP variant found in a cell or subject. For example, guides in Table 6 having “7984A” in their name can be used when the cell or subject has an “A” at the position of the rs7984 SNP, while guides having “7984G” in their name can be used when the cell or subject has a “G” at the position of the rs7984 SNP. Such guides can be used, for example, with a guide RNA targeting RHO intron 1 to knock-out expression of a mutant RHO protein. Dual targeting approaches to RHO editing are described in WO 2023/285431, the contents of which are incorporated herein by reference in their entireties.

In some embodiments, a gRNA of the disclosure has a spacer whose nucleotide sequence comprises 15 or more consecutive nucleotides from a sequence shown in Table 6. In some embodiments, a gRNA of the disclosure has a spacer whose nucleotide sequence comprises 16 or more consecutive nucleotides from a sequence shown in Table 6. In some embodiments, a gRNA of the disclosure has a spacer whose nucleotide sequence comprises 17 or more consecutive nucleotides from a sequence shown in Table 6. In some embodiments, a gRNA of the disclosure has a spacer whose nucleotide sequence comprises 18 or more consecutive nucleotides from a sequence shown in Table 6. In some embodiments, a gRNA of the disclosure has a spacer whose nucleotide sequence comprises 19 or more consecutive nucleotides from a sequence shown in Table 6. In some embodiments, a gRNA of the disclosure has a spacer whose nucleotide sequence comprises 20 or more consecutive nucleotides from a sequence shown in Table 6. In some embodiments, a gRNA of the disclosure has a spacer whose nucleotide sequence comprises 21 or more consecutive nucleotides from a sequence shown in Table 6. In some embodiments, a gRNA of the disclosure has a spacer whose nucleotide sequence comprises 22 or more consecutive nucleotides from a sequence shown in Table 6. In some embodiments, a gRNA of the disclosure has a spacer whose nucleotide sequence comprises 23 or more consecutive nucleotides from a sequence shown in Table 6. In some embodiments, a gRNA of the disclosure has a spacer whose nucleotide sequence comprises 24 consecutive nucleotides from a sequence shown in Table 6.

6.3.2. sgRNA Molecules

gRNAs of the disclosure can be single-guide RNA (sgRNA) molecules. A sgRNA can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

The sgRNA can comprise a variable length spacer sequence (e.g., 15 to 30 nucleotides) at the 5′ end of the sgRNA sequence and a 3′ sgRNA segment.

Type II Cas gRNAs typically comprise a repeat-antirepeat duplex and/or one or more stem-loops generated by the gRNA's secondary structure. The length of the repeat-antirepeat duplex and/or one or more stem-loops can be modified in order to modulate (e.g., increase) the editing efficacy of a Type II Cas nuclease, and/or to reduce the size of a guide RNA for easier vectorization in situations in which the cargo size of the vector is limiting (e.g., AAV vectors).

For example, the repeat-antirepeat duplex (which in a sgRNA is fused through a synthetic linker to become an additional stem loop in the structure) can be trimmed at different lengths without generally having detrimental effects on nuclease function and in some cases even producing increased enzymatic activity. If bulges are present within this duplex they generally should be retained in the final guide RNA sequence.

Further optimization of the structure can be obtained by introducing targeted base changes into the stems of the gRNA to increase their stability and folding. Such base changes will preferably correspond to the introduction of G:C couples, which are known to generate the strongest Watson-Crick pairing. For the sake of clarity, these substitutions can consist in the introduction of a G or a C in a specific position of a stem together with a complementary substitution in another position of the gRNA sequence which is predicted to base pair with the former, for example according to available bioinformatic tools for RNA folding such as UNAfold or RNAfold.

Stem-loop trimming can also be exploited to stabilize desired secondary structures by removing portions of the guide RNA producing unwanted secondary structures through annealing with other regions of the RNA molecule.

Exemplary 3′ sgRNA scaffold sequences for Type IIA Cas sgRNAs are shown in Table 7A. Exemplary 3′ sgRNA scaffold sequences for Type IIC Cas sgRNAs are shown in Table 7B.

TABLE 7A
Sequences of sgRNA Scaffolds for Type IIA Cas Proteins

| | | SEQ ID |
|---|---|---|
| Name | Sequence | NO: |
| AHZW Type II Cas | GUUCUGCUACCAUCGAAAUUUUUGCUAGGCUACAAGAAAUUGUAGU | 77 |
| sgRNA scaffold | CUAGCAAAGGUUUUGAUGAUCUAGCAGAACAAGGGUUUAUCCCGGA | |
| | AUCGACUCCUUAGGGAGUC | |
| AHZW Type II Cas | GUUCUGCUACCAUCGAAAGAUGAUCUAGCAGAACAAGGGUUUAUCC | 78 |
| sgRNA scaffold | CGGAAUCGACUCCUUAGGGAGUC | |
| (trimmed) | | |
| ABSE Type II Cas | GUUUUGGUACCCUCUAAAUUUUUGCUAUACUGAAAAGUAUAGCAAAG | 79 |
| sgRNA | GUUUAGAGGACCUAUCAAAACAAGGGAAUUAUUCCCGAAAUCGGAAC | |
| scaffold | UGCUAAGCAGUUCC | |
| ABSE Type II Cas | GUUUUGGUACCCUCGAAAGAGGACCUAUCAAAACAAGGGAAUUAUU | 80 |
| sgRNA | CCCGAAAUCGGAACUGCUAAGCAGUUCC | |
| scaffold (trimmed) | | |
| AIXM Type II Cas | GUUUUGCUACCCUCACAAUUUUUGCUAUGUAAGAAAUUACAUAGCAA | 81 |
| sgRNA scaffold | AGAUUGUGAGGAUCUAGCGAAACAAGGGCUGUCUUAGGACAUGUCC | |
| | CGAAAUCGAGUCUCGUAGGAGACUC | |
| AIXM Type II Cas | GUUUUGCUACCCUCGAAAGAGGAUCUAGCGAAACAAGGGCUGUCUU | 82 |
| sgRNA scaffold | AGGACAUGUCCCGAAAUCGAGUCUCGUAGGAGACUC | |
| (trimmed) | | |
| AXTQ Type II Cas | GUUUUAGUACCUGAAAGAAUUGAGUUAUUGUAAAACGAAAGUUUUG | 83 |
| sgRNA scaffold | CAAUAACUCAAUUUUUUCAGAUCUACUAAAACAAGGCUUUAUGCCGA | |
| | AAUCAAGGACACAGAUAAGUGUCC | |
| AXTQ Type II Cas | GUUUUAGUACCUGAGAAAUCAGAUCUACUAAAACAAGGCUUUAUGCC | 84 |
| sgRNA scaffold | GAAAUCAAGGACACAGAUAAGUGUCC | |
| (trimmed) | | |
| AIWM* Type II | GUUUUGCUACCCUCACAAUUUUUGCUAUGUAAGAAAUUACAUAGCAA | 85 |
| Cas sgRNA | AGAUUGUGAGGAUCUAGCAAAACAAGGGCUGUCUUAGGACAUGUCC | |
| scaffold | CGAAAUCGAGUCUCGUAGGAGACUC | |
| AIWM* Type II | GUUUUGCUACCCUCGAAAGAGGAUCUAGCAAAACAAGGGCUGUCUU | 86 |
| Cas sgRNA | AGGACAUGUCCCGAAAUCGAGUCUCGUAGGAGACUC | |
| scaffold (trimmed) | | |

*AIWM, AIWR, and AIYQ Type II Cas proteins can use the same sgRNA scaffold.

TABLE 7B
Sequences of sgRNA Scaffolds for Type IIC Cas Proteins

| | | SEQ ID |
|---|---|---|
| Name | Sequence | NO: |
| EQSC Type II | GUCUUGAGCUCGCACUUUUCCCCAAGCUGAGAAAUCACCUUGGGGAAA | 87 |
| Cas sgRNA | AGUGCGAGACUCCAGACAAGGGGAACCUACAACGGUAGGUUCACCCGU | |
| scaffold | AGGGUUACCCCCGCGUCAUCUUCGGAAGGCGCGGGGCGAACUC | |
| EQSC Type II | GUCUUGAGCUCGGAAACGAGACUCCAGACAAGGGGAACCUACAACGGU | 88 |
| Cas sgRNA | AGGUUCACCCGUAGGGUUACCCCCGCGUCAUCUUCGGAAGGCGCGGG | |
| scaffold | GCGAACUC | |
| (trimmed) | | |
| BDLP Type II | GUCUUGAGCUCGCACUUUUCCCCAAGCUGAGAAAUCACCUUGGGGAAA | 89 |
| Cas sgRNA | AGUGCGAGACUCCAGACAAGGGGAGUCUACAACAGUAGGUUCACCCGU | |
| scaffold | AGGGUUACCCCCGCGUCAUCCUCGGAAGGCGCGGGGCGAACUC | |
| BDLP Type II | GUCUUGAGCUCGGAAACGAGACUCCAGACAAGGGGAGUCUACAACAGU | 90 |
| Cas sgRNA | AGGUUCACCCGUAGGGUUACCCCCGCGUCAUCCUCGGAAGGCGCGGG | |
| scaffold | GCGAACUC | |
| (trimmed) | | |
| BDKL Type II | GUCUUGAGUUUGCGCCCUUCCCCAAGGUGAGAAAUCACCUUGGGGAA | 91 |
| Cas sgRNA | GGGCGCUGCUCCAGACAAGGGAAGCCACUUGCUGGCUUACCCGUAAA | |
| scaffold | GUUUCAACCCCGCGUUGCCUUCAGGCGGCGCGGGGUGAAC | |
| BDKL Type II | GUCUUGAGUUUGCGGAAACGCUGCUCCAGACAAGGGAAGCCACUUGC | 92 |
| Cas sgRNA | UGGCUUACCCGUAAAGUUUCAACCCCGCGUUGCCUUCAGGCGGCGCG | |
| scaffold | GGGUGAAC | |
| (trimmed) | | |

The sgRNA (e.g., for use with AHZW Type II Cas proteins, ABSE Type II Cas proteins, AIXM Type II Cas proteins, AXTQ Type II Cas proteins, AIWM Type II Cas proteins, AIWR Type II Cas proteins, AIYQ Type II Cas Proteins, EQSC Type II proteins, BDLP Type II proteins, or BDKL Type II B proteins) can comprise no uracil base at the 3′ end of the sgRNA sequence. Typically, however, the sgRNA comprises one or more uracil bases at the 3′ end of the sgRNA sequence, for example to promote correct sgRNA folding. For example, the sgRNA can comprise 1 uracil (U) at the 3′ end of the sgRNA sequence. The sgRNA can comprise 2 uracil (UU) at the 3′ end of the sgRNA sequence. The sgRNA can comprise 3 uracil (UUU) at the 3′ end of the sgRNA sequence. The sgRNA can comprise 4 uracil (UUUU) at the 3′ end of the sgRNA sequence. The sgRNA can comprise 5 uracil (UUUUU) at the 3′ end of the sgRNA sequence. The sgRNA can comprise 6 uracil (UUUUUU) at the 3′ end of the sgRNA sequence. The sgRNA can comprise 7 uracil (UUUUUUU) at the 3′ end of the sgRNA sequence. The sgRNA can comprise 8 uracil (UUUUUUUU) at the 3′ end of the sgRNA sequence. Different length stretches of uracil can be appended at the 3′end of a sgRNA as terminators. Thus, for example, the 3′ sgRNA sequences set forth in Table 7A and Table 7B can be modified by adding (or removing) one or more uracils at the end of the sequence.

In some embodiments, a sgRNA scaffold for use with an AHZW Type II Cas protein comprises the sequence

(SEQ ID NO: 93)
GUUCUGCUACCAUCGAAAUUUUUGCUAGGCUACAAGAAAUUGUAGUCUA
GCAAAGGUUUUGAUGAUCUAGCAGAACAAGGGUUUAUCCCGGAAUCGAC
UCCUUAGGGAGUCUUUUUU.

In some embodiments, a sgRNA scaffold for use with an AHZW Type II Cas protein comprises the sequence

(SEQ ID NO: 94)
GUUCUGCUACCAUCGAAAGAUGAUCUAGCAGAACAAGGGUUUAUCCCGG
AAUCGACUCCUUAGGGAGUCUUUUUU.

In some embodiments, a sgRNA scaffold for use with an ABSE Type II Cas protein comprises the sequence GUUUUGGUACCCUCUAAAUUUUUGCUAUACUGAAAAGUAUAGCAAAGGUUUAGAGGACCUAUCAA AACAAGGGAAUUAUUCCCGAAAUCGGAACUGCUAAGCAGUUCCUUUUUU (SEQ ID NO:95). In some embodiments, a sgRNA scaffold for use with an ABSE Type II Cas protein comprises the sequence

(SEQ ID NO: 96)
GUUUUGGUACCCUCGAAAGAGGACCUAUCAAAACAAGGGAAUUAUUCCC
GAAAUCGGAACUGCUAAGCAGUUCCUUUUUU.

In some embodiments, a sgRNA scaffold for use with an AIXM Type II Cas protein comprises the sequence GUUUUGCUACCCUCACAAUUUUUGCUAUGUAAGAAAUUACAUAGCAAAGAUUGUGAGGAUCUAGC GAAACAAGGGCUGUCUUAGGACAUGUCCCGAAAUCGAGUCUCGUAGGAGACUCUUUUUU (SEQ ID NO:97). In some embodiments, a sgRNA scaffold for use with an AIXM Type II Cas protein comprises the sequence

(SEQ ID NO: 98)
GUUUUGCUACCCUCGAAAGAGGAUCUAGCGAAACAAGGGCUGUCUUAGG
ACAUGUCCCGAAAUCGAGUCUCGUAGGAGACUCUUUUUU.

In some embodiments, a sgRNA scaffold for use with an AXTQ Type II Cas protein comprises the sequence GUUUUAGUACCUGAAAGAAUUGAGUUAUUGUAAAACGAAAGUUUUGCAAUAACUCAAUUUUUUCA GAUCUACUAAAACAAGGCUUUAUGCCGAAAUCAAGGACACAGAUAAGUGUCCUUUUUU (SEQ ID NO:99). In some embodiments, a sgRNA scaffold for use with an AXTQ Type II Cas protein comprises the sequence

(SEQ ID NO: 100)
GUUUUAGUACCUGAGAAAUCAGAUCUACUAAAACAAGGCUUUAUGCCGA
AAUCAAGGACACAGAUAAGUGUCCUUUUUU.

In some embodiments, a sgRNA scaffold for use with an AIWM Type II Cas protein, AIWR Type II Cas protein, or AIYQ Type II Cas protein comprises the sequence GUUUUGCUACCCUCACAAUUUUUGCUAUGUAAGAAAUUACAUAGCAAAGAUUGUGAGGAUCUAGC AAAACAAGGGCUGUCUUAGGACAUGUCCCGAAAUCGAGUCUCGUAGGAGACUCUUUUUU (SEQ ID NO:101). In some embodiments, a sgRNA scaffold for use with an AIWM Type II Cas protein, AIWR Type II Cas protein, or AIYQ Type II Cas protein comprises the sequence

(SEQ ID NO: 102)
GUUUUGCUACCCUCGAAAGAGGAUCUAGCAAAACAAGGGCUGUCUUAGG
ACAUGUCCCGAAAUCGAGUCUCGUAGGAGACUCUUUUUU.

In some embodiments, a sgRNA scaffold for use with an EQSC Type II Cas protein comprises the sequence GUCUUGAGCUCGCACUUUUCCCCAAGCUGAGAAAUCACCUUGGGGAAAAGUGCGAGACUCCAGA CAAGGGGAACCUACAACGGUAGGUUCACCCGUAGGGUUACCCCCGCGUCAUCUUCGGAAGGCGC GGGGCGAACUCUUUUUU (SEQ ID NO:103). In some embodiments, a sgRNA scaffold for use with an EQSC Type II Cas protein comprises the sequence

(SEQ ID NO: 104)
GUCUUGAGCUCGGAAACGAGACUCCAGACAAGGGGAACCUACAACGGUA
GGUUCACCCGUAGGGUUACCCCCGCGUCAUCUUCGGAAGGCGCGGGGCG
AACUCUUUUUU.

In some embodiments, a sgRNA scaffold for use with an BDLP Type II Cas protein comprises the sequence GUCUUGAGCUCGCACUUUUCCCCAAGCUGAGAAAUCACCUUGGGGAAAAGUGCGAGACUCCAGA CAAGGGGAGUCUACAACAGUAGGUUCACCCGUAGGGUUACCCCCGCGUCAUCCUCGGAAGGCGC GGGGCGAACUCUUUUUU (SEQ ID NO:105). In some embodiments, a sgRNA scaffold for use with an BDLP Type II Cas protein comprises the sequence

(SEQ ID NO: 106)
GUCUUGAGCUCGGAAACGAGACUCCAGACAAGGGGAGUCUACAACAGUA
GGUUCACCCGUAGGGUUACCCCCGCGUCAUCCUCGGAAGGCGCGGGGCG
AACUCUUUUUU.

In some embodiments, a sgRNA scaffold for use with an BDKL Type II Cas protein comprises the sequence GUCUUGAGUUUGCGCCCUUCCCCAAGGUGAGAAAUCACCUUGGGGAAGGGCGCUGCUCCAGACA AGGGAAGCCACUUGCUGGCUUACCCGUAAAGUUUCAACCCCGCGUUGCCUUCAGGCGGCGCGG GGUGAACUUUUUU (SEQ ID NO:107). In some embodiments, a sgRNA scaffold for use with an EQSC Type II Cas protein comprises the sequence

(SEQ ID NO: 108)
GUCUUGAGUUUGCGGAAACGCUGCUCCAGACAAGGGAAGCCACUUGCUG
GCUUACCCGUAAAGUUUCAACCCCGCGUUGCCUUCAGGCGGCGCGGGGU
GAACUUUUUU.

6.3.3. Modified gRNA Molecules

Guide RNAs can be readily synthesized by chemical means, enabling a number of modifications to be readily incorporated, as described in the art. The disclosed gRNA (e.g., sgRNA) molecules can be unmodified or can contain any one or more of an array of chemical modifications.

While chemical synthetic procedures are continually expanding, purifications of such RNAs by procedures such as high-performance liquid chromatography (HPLC, which avoids the use of gels such as PAGE) tends to become more challenging as polynucleotide lengths increase significantly beyond a hundred or so nucleotides. One approach that can be used for generating chemically modified RNAs of greater length is to produce two or more molecules that are ligated together. Much longer RNAs, such as those encoding a Type II Cas endonuclease, are more readily generated enzymatically. While fewer types of modifications are available for use in enzymatically produced RNAs, there are still modifications that can be used to, for instance, enhance stability, reduce the likelihood or degree of innate immune response, and/or enhance other attributes, as described herein and in the art.

By way of illustration of various types of modifications, especially those used frequently with smaller chemically synthesized RNAs, modifications can comprise one or more nucleotides modified at the 2′ position of the sugar, for instance a 2′-O-alkyl, 2′-O-alkyl-O-alkyl, or 2′-fluoro-modified nucleotide. In some examples, RNA modifications can comprise 2′-fluoro, 2′-amino or 2′-O-methyl modifications on the ribose of pyrimidines, abasic residues, or an inverted base at the 3′ end of the RNA. Such modifications can be routinely incorporated into oligonucleotides and these oligonucleotides have been shown to have a higher Tm (thus, higher target binding affinity) than 2′-deoxyoligonucleotides against a given target.

A number of nucleotide and nucleoside modifications have been shown to make the oligonucleotide into which they are incorporated more resistant to nuclease digestion than the native oligonucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH,˜N(CH₃)—O—CH₂ (known as a methylene (methylimino) or MMI backbone), CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones (see De Mesmaeker et al. 1995, Ace. Chem. Res., 28:366-374); morpholino backbone structures (see U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., 1991, Science 254:1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Braasch and David Corey, 2002, Biochemistry, 41(14):4503-4510; Genesis, Volume 30, Issue 3, (2001); Heasman, 2002, Dev. Biol., 243: 209-214; Nasevicius et al., 2000, Nat. Genet., 26:216-220; Lacerra et al., 2000, Proc. Natl. Acad. Sci., 97: 9591-9596; and U.S. Pat. No. 5,034,506.

Cyclohexenyl nucleic acid oligonucleotide mimetics are described in Wang et al., 2000, J. Am. Chem. Soc., 122: 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic intemucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

One or more substituted sugar moieties can also be included, e.g., one of the following at the 2′ position: OH, SH, SCH₃, F, OCN, OCH₃, OCH₃ O(CH₂)n CH₃, O(CH₂)n NH₂, or O(CH₂)n CH₃, where n is from 1 to about 10; C₁ to C₁₀ lower alkyl, alkoxyalkoxy, substituted lower alkyl, alkaryl or aralkyl; Cl; Br; CN; CF₃; OCF₃; O—, S—, or bi-alkyl; O—, S—, or N-alkenyl; SOCH₃; SO₂ CH₃; ONO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. In some aspects, a modification includes 2′-methoxyethoxy (2′-O—CH₂CH₂OCH₃, also known as 2′-O-(2-methoxyethyl)) (Martin et al., 1995, Helv. Chim. Acta, 78, 486). Other modifications include 2′-methoxy (2′-O—CH₃), 2′-propoxy (2′-OCH₂ CH₂CH₃) and 2′-fluoro (2′-F). Similar modifications can also be made at other positions on the oligonucleotide, particularly the 3′ position of the sugar on the 3′ terminal nucleotide and the 5′ position of 5′ terminal nucleotide. Oligonucleotides can also have sugar mimetics, such as cyclobutyls in place of the pentofuranosyl group.

In some examples, both a sugar and an internucleoside linkage (in the backbone) of the nucleotide units can be replaced with novel groups. The base units can be maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide can be replaced with an amide containing backbone, for example, an aminoethylglycine backbone. The nucleobases can be retained and bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262. Further teaching of PNA compounds can be found in Nielsen et al., 1991, Science, 254: 1497-1500.

RNAs such as guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxy cytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl) adenine, and 2,6-diaminopurine. Komberg, A., DNA Replication, W. H. Freeman & Co., San Francisco, pp. 75-77 (1980); Gebeyehu et al., Nucl. Acids Res. 15:4513 (1997). A “universal” base known in the art, e.g., inosine, can also be included. 5-Me-C substitutions have been shown to increase nucleic acid duplex stability by about 0.6-1.2° C. (Sanghvi, Y. S., in Crooke, S. T. and Lebleu, B., eds., Antisense Research and Applications, CRC Press, Boca Raton, 1993, pp. 276-278) and are aspects of base substitutions.

Modified nucleobases can comprise other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

Further, nucleobases can comprise those disclosed in U.S. Pat. No. 3,687,808, those disclosed in ‘The Concise Encyclopedia of Polymer Science and Engineering’, 858-859, Kroschwitz, J. I., ed. John Wiley & Sons, 1990, those disclosed by Englisch et al., Angewandle Chemie, International Edition’, 1991, 30, p. 613, and those disclosed by Sanghvi, Y. S., Chapter 15, Antisense Research and Applications', 289-302, Crooke, S. T. and Lebleu, B. ea., CRC Press, 1993. Certain of these nucleobases can be useful for increasing the binding affinity of the oligomeric compounds of the invention. These include 5-substituted pyrimidines, 6-azapyrimidines and N-2, N-6 and O-6 substituted purines, comprising 2-aminopropyladenine, 5-propynyluracil and 5-propynylcytosine. 5-methylcytosine substitutions have been shown to increase nucleic acid duplex stability by about 0.6-1.2° C. (Sanghvi, Y. S., Crooke, S. T. and Lebleu, B., eds, ‘Antisense Research and Applications’, CRC Press, Boca Raton, 1993, 276-278) and are aspects of base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and U.S. Patent Application Publication 2003/0158403.

Thus, a modified gRNA can include, for example, one or more non-natural sugars, internucleotide linkages and/or bases. It is not necessary for all positions in a given gRNA to be uniformly modified, and in fact more than one of the aforementioned modifications can be incorporated in a single oligonucleotide, or even in a single nucleoside within an oligonucleotide.

The guide RNAs and/or mRNA (or DNA) encoding an endonuclease can be chemically linked to one or more moieties or conjugates that enhance the activity, cellular distribution, or cellular uptake of the oligonucleotide. Such moieties comprise, but are not limited to, lipid moieties such as a cholesterol moiety (Letsinger et al. 1989, Proc. Natl. Acad. Sci. USA, 86: 6553-6556); cholic acid (Manoharan et al, 1994, Bioorg. Med. Chem. Let., 4: 1053-1060); a thioether, e.g., hexyl-S-tritylthiol (Manoharan et al, 1992, Ann. N. Y. Acad. Sci., 660: 306-309; Manoharan et al., 1993, Bioorg. Med. Chem. Let., 3: 2765-2770); a thiocholesterol (Oberhauser et al., 1992, Nucl. Acids Res., 20: 533-538); an aliphatic chain, e.g., dodecandiol or undecyl residues (Kabanov et al, 1990, FEBS Lett., 259: 327-330; Svinarchuk et al, 1993, Biochimie, 75: 49-54); a phospholipid, e.g., di-hexadecyl-rac-glycerol or triethylammonium 1,2-di-O-hexadecyl-rac-glycero-3-H-phosphonate (Manoharan et al., 1995, Tetrahedron Lett., 36: 3651-3654; and Shea et al, 1990, Nucl. Acids Res., 18: 3777-3783); a polyamine or a polyethylene glycol chain (Mancharan et al, 1995, Nucleosides & Nucleotides, 14: 969-973); adamantane acetic acid (Manoharan et al, 1995, Tetrahedron Lett., 36: 3651-3654); a palmityl moiety (Mishra et al., 1995, Biochim. Biophys. Acta, 1264: 229-237); or an octadecylamine or hexylamino-carbonyl-t oxycholesterol moiety (Crooke et al, 1996, J. Pharmacol. Exp. Ther., 277: 923-937). See also U.S. Pat. Nos. 4,828,979; 4,948,882; 5,218,105; 5,525,465; 5,541,313; 5,545,730; 5,552,538; 5,578,717; 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241; 5,391,723; 5,416,203; 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Sugars and other moieties can be used to target proteins and complexes comprising nucleotides, such as cationic polysomes and liposomes, to particular sites. For example, hepatic cell directed transfer can be mediated via asialoglycoprotein receptors (ASGPRs); see, e.g., Hu, et al., 2014, Protein Pept Lett. 21(10):1025-30. Other systems known in the art and regularly developed can be used to target biomolecules of use in the present case and/or complexes thereof to particular target cells of interest.

Targeting moieties or conjugates can include conjugate groups covalently bound to functional groups, such as primary or secondary hydroxyl groups. Conjugate groups of the present disclosure include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this present disclosure, include groups that improve uptake, enhance resistance to degradation, and/or strengthen sequence-specific hybridization with the target nucleic acid. Groups that enhance the pharmacokinetic properties, in the context of this disclosure, include groups that improve uptake, distribution, metabolism or excretion of the compounds of the present disclosure. Representative conjugate groups are disclosed in International Patent Application Publication WO1993007883, and U.S. Pat. No. 6,287,860. Conjugate moieties include, but are not limited to, lipid moieties such as a cholesterol moiety, cholic acid, a thioether, e.g., hexyl-5-trityl thiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241; 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

A large variety of modifications have been developed and applied to enhance RNA stability, reduce innate immune responses, and/or achieve other benefits that can be useful in connection with the introduction of polynucleotides into human cells, as described herein; see, e.g., the reviews by Whitehead K A et al., 2011, Annual Review of Chemical and Biomolecular Engineering, 2: 77-96; Gaglione and Messere, 2010, Mini Rev Med Chem, 10(7):578-95; Chernolovskaya et al, 2010, Curr Opin Mol Ther., 12(2): 158-67; Deleavey et al., 2009, Curr Protoc Nucleic Acid Chem Chapter 16:Unit 16.3; Behlke, 2008, Oligonucleotides 18(4):305-19; Fucini et al, 2012, Nucleic Acid Ther 22(3): 205-210; Bremsen et al, 2012, Front Genet 3: 154.

### 6.4. Systems

The disclosure provides systems comprising a Type II Cas protein of the disclosure (e.g., as described in Section 6.2) and a means for targeting the Type II Cas protein to a target genomic sequence. The means for targeting the Type II Cas protein to a target genomic sequence can be a guide RNA (gRNA) (e.g., as described in Section 6.3).

The disclosure also provides systems comprising a Type II Cas protein of the disclosure (e.g., as described in Section 6.2) and a gRNA (e.g., as described in Section 6.3). The systems can comprise a ribonucleoprotein particle (RNP) in which a Type II Cas protein is complexed with a gRNA, for example a sgRNA or separate crRNA and tracrRNA. Systems of the disclosure can in some embodiments further comprise genomic DNA complexed with the Type II Cas protein and the gRNA. Accordingly, the disclosure provides systems comprising a Type II Cas protein, a genomic DNA, and gRNA, all complexed with one another.

The systems of the disclosure can exist within a cell (whether the cell is in vivo, ex vivo, or in vitro) or outside a cell (e.g., in a particle our outside of a particle).

### 6.5. Nucleic Acids

The disclosure provides nucleic acids (e.g., DNA or RNA) encoding Type II Cas proteins (e.g., AHZW Type II Cas proteins, ABSE Type II Cas proteins, AIXM Type II Cas proteins, AXTQ Type II Cas proteins, AIWM Type II Cas proteins, AIWR Type II proteins, AIYQ Type II proteins, EQSC Type II proteins, BDLP Type II proteins, and BDKL Type II proteins), nucleic acids encoding gRNAs of the disclosure, nucleic acids encoding both Type II Cas proteins and gRNAs, and pluralities of nucleic acids, for example comprising a nucleic acid encoding a Type II Cas protein and a gRNA.

A nucleic acid encoding a Type II Cas protein and/or gRNA can be, for example, a plasmid or a viral genome (e.g., a lentivirus, retrovirus, adenovirus, or adeno-associated virus genome). Plasmids can be, for example, plasmids for producing virus particles, e.g., lentivirus particles, or plasmids for propagating the Type II Cas and gRNA coding sequences in bacterial (e.g., E. coli) or eukaryotic (e.g., yeast) cells.

A nucleic acid encoding a Type II Cas protein can, in some embodiments, further encode a gRNA. Alternatively, a gRNA can be encoded by a separate nucleic acid (e.g., DNA or mRNA).

Nucleic acids encoding a Type II Cas protein can be codon optimized, e.g., where at least one non-common codon or less-common codon has been replaced by a codon that is common in a host cell. For example, a codon optimized nucleic acid can direct the synthesis of an optimized messenger mRNA, e.g., optimized for expression in a mammalian expression system. As an example, if the intended target nucleic acid is within a human cell, a human codon-optimized polynucleotide encoding Type II Cas can be used for producing a Type II Cas polypeptide. Exemplary codon-optimized sequences are shown in Tables 1A-1G and Tables 2A-2C.

Nucleic acids of the disclosure, e.g., plasmids and viral vectors, can comprise one or more regulatory elements such as promoters, enhancers, and other expression control elements (e.g., transcription termination signals, such as polyadenylation signals and poly-U sequences). Such regulatory elements are described, for example, in Goeddel, 1990, GENE EXPRESSION TECHNOLOGY: METHODS IN ENZYMOLOGY 185, Academic Press, San Diego, Calif. Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest or in particular cell types. Regulatory elements may also direct expression in a temporal-dependent manner, such as in a cell-cycle dependent or developmental stage-dependent manner, which may or may not also be tissue or cell-type specific. In some embodiments, a nucleic acid of the disclosure comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof, e.g., to express a Type II Cas protein and a gRNA separately. Examples of pol III promoters include, but are not limited to, U6 and H1 promoters. Examples of pol II promoters include, but are not limited to, the retroviral Rous Sarcoma virus (RSV) LTR promoter (optionally with the RSV enhancer), the cytomegalovirus (CMV) promoter (optionally with the CMV enhancer) (see, e.g., Boshart et al, 1985, Cell 41:521-530), the SV40 promoter, the dihydrofolate reductase promoter, the β-actin promoter, the phosphoglycerol kinase (PGK) promoter, and EF1α promoters (for example, full length EF1α promoter and the EFS promoter, which is a short, intron-less form of the full EF1α promoter). Exemplary enhancer elements include WPRE; CMV enhancers; the R-U5′ segment in LTR of HTLV-I; SV40 enhancer; and the intron sequence between exons 2 and 3 of rabbit β-globin. It will be appreciated by those skilled in the art that the design of an expression vector can depend on such factors as the choice of the host cell, the level of expression desired, etc.

The term “vector” refers to a polynucleotide molecule capable of transporting another nucleic acid to which it has been linked. One type of polynucleotide vector includes a “plasmid”, which refers to a circular double-stranded DNA loop into which additional nucleic acid segments are or can be ligated. Another type of polynucleotide vector is a viral vector; wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some examples, vectors can be capable of directing the expression of nucleic acids to which they are operably linked. Such vectors can be referred to herein as “recombinant expression vectors”, or more simply “expression vectors”, which serve equivalent functions.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, CA (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

Vectors can include, but are not limited to, viral vectors based on vaccinia virus, poliovirus, adenovirus, adeno-associated virus (e.g., AAV2, AAV5, AAV7m8, AAV8, AAV9, AAVrh8r, AAVrh10), SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXTI, pSG5, pSVK3, pBPV, pMSG, and pSVLSV40 (Pharmacia). Additional vectors contemplated for eukaryotic target cells include, but are not limited to, the vectors pCTx-I, pCTx-2, and pCTx-3. Other vectors can be used so long as they are compatible with the host cell.

In some examples, a vector can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. The vector can be a self-inactivating vector that either inactivates the viral sequences or the components of the CRISPR machinery or other elements.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-l promoters (for example, the full EF1α promoter and the EFS promoter), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-l.

An expression vector can also contain a ribosome binding site for translation initiation and a transcription terminator. The expression vector can also comprise appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some cases, the promoter can be a spatially restricted and/or temporally restricted promoter (e.g., a tissue specific promoter, for example a human RHO promoter or human rhodopsin kinase promoter (hGRK), a cell type specific promoter, etc.).

### 6.6. Particles and Cells

The disclosure further provides particles comprising a Type II Cas protein of the disclosure (e.g., an AHZW Type II Cas protein, an ABSE Type II Cas protein, an AIXM Type II Cas protein, an AXTQ Type II Cas protein, an AIWM Type II Cas protein, an AIWR Type II protein, an AIYQ Type II protein, an EQSC Type II protein, a BDLP Type II protein, or a BDKL Type II protein), particles comprising a gRNA of the disclosure, particles comprising a system of the disclosure, and particles comprising a nucleic acid or plurality of nucleic acids of the disclosure. The particles can in some embodiments comprise or further comprise a gRNA, or a nucleic acid encoding the gRNA (e.g., DNA or mRNA). For example, the particles can comprise a RNP of the disclosure. Exemplary particles include lipid nanoparticles, vesicles, viral-like particles (VLPs) and gold nanoparticles. See, e.g., WO 2020/012335, the contents of which are incorporated herein by reference in their entireties, which describes vesicles that can be used to deliver gRNA molecules and Type II Cas proteins to cells (e.g., complexed together as a RNP).

The disclosure provides particles (e.g., virus particles) comprising a nucleic acid encoding a Type II Cas protein of the disclosure. The particles can further comprise a nucleic acid encoding a gRNA. Alternatively, a nucleic acid encoding a Type II Cas protein can further encode a gRNA.

The disclosure further provides pluralities of particles (e.g., pluralities of virus particles). Such pluralities can include a particle encoding a Type II Cas protein and a different particle encoding a gRNA. For example, a plurality of particles can comprise a virus particle (e.g., a AAV2, AAV5, AAV7m8, AAV8, AAV9, AAVrh8r, or AAVrh10 virus particle) encoding a Type II Cas protein and a second virus particle (e.g., a AAV2, AAV5, AAV7m8, AAV8, AAV9, AAVrh8r, or AAVrh10 virus particle) encoding a gRNA. Alternatively, a plurality of particles can comprise a plurality of virus particles where each particle encodes a Type II Cas protein and a gRNA.

The disclosure further provides cells and populations of cells (e.g., ex vivo cells and populations of cells) that can comprise a Type II Cas protein (e.g., introduced to the cell as a RNP) or a nucleic acid encoding the Type II Cas protein (e.g., DNA or mRNA) (optionally also encoding a gRNA). The disclosure further provides cells and populations of cells comprising a gRNA of the disclosure (optionally complexed with a Type II Cas protein) or a nucleic acid encoding the gRNA (e.g., DNA or mRNA) (optionally also encoding a Type II Cas protein). The cells and populations of cells can be, for example, human cells such as a stem cell, e.g., a hematopoietic stem cell (HSC), a pluripotent stem cell, an induced pluripotent stem cell (iPS), or an embryonic stem cell. In some embodiments, the cells and populations of cells are T cells. Methods for introducing proteins and nucleic acids to cells are known in the art. For example, a RNP can be produced by mixing a Type II Cas protein and one or more guide RNAs in an appropriate buffer. An RNP can be introduced to a cell, for example, via electroporation and other methods known in the art.

The cell populations of the disclosure can be cells in which gene editing by the systems of the disclosure has taken place, or cells in which the components of a system of the disclosure have been introduced or expressed but gene editing has not taken place, or a combination thereof. A cell population can comprise, for example, a population in which at least 1%, at least 5%, at least 10%, at least 15%, at least 20%, at least 30%, at least 40%, at least 50%, at least 60%, or at least 70% of the cells have undergone gene editing by a system of the disclosure.

### 6.7. Pharmaceutical Compositions

Also disclosed herein are pharmaceutical formulations and medicaments comprising a Type II Cas protein, gRNA, nucleic acid or plurality of nucleic acids, system, particle, or plurality of particles of the disclosure together with a pharmaceutically acceptable excipient.

Suitable excipients include, but are not limited to, salts, diluents, (e.g., Tris-HCl, acetate, phosphate), preservatives (e.g., Thimerosal, benzyl alcohol, parabens), binders, fillers, solubilizers, disintegrants, sorbents, solvents, pH modifying agents, antioxidants, antinfective agents, suspending agents, wetting agents, viscosity modifiers, tonicity agents, stabilizing agents, and other components and combinations thereof. Suitable pharmaceutically acceptable excipients can be selected from materials which are generally recognized as safe (GRAS), and may be administered to an individual without causing undesirable biological side effects or unwanted interactions. Suitable excipients and their formulations are described in Remington's Pharmaceutical Sciences, 16th ed. 1980, Mack Publishing Co. In addition, such compositions can be complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Suitable dosage forms for administration, e.g., parenteral administration, include solutions, suspensions, and emulsions.

The components of the pharmaceutical formulation can be dissolved or suspended in a suitable solvent such as, for example, water, Ringer's solution, phosphate buffered saline (PBS), or isotonic sodium chloride. The formulation may also be a sterile solution, suspension, or emulsion in a nontoxic, parenterally acceptable diluent or solvent such as 1,3-butanediol.

In some cases, formulations can include one or more tonicity agents to adjust the isotonic range of the formulation. Suitable tonicity agents are well known in the art and include glycerin, mannitol, sorbitol, sodium chloride, and other electrolytes. In some cases, the formulations can be buffered with an effective amount of buffer necessary to maintain a pH suitable for parenteral administration. Suitable buffers are well known by those skilled in the art and some examples of useful buffers are acetate, borate, carbonate, citrate, and phosphate buffers.

In some embodiments, the formulation can be distributed or packaged in a liquid form, or alternatively, as a solid, obtained, for example by lyophilization of a suitable liquid formulation, which can be reconstituted with an appropriate carrier or diluent prior to administration. In some embodiments, the formulations can comprise a guide RNA and a Type II Cas protein in a pharmaceutically effective amount sufficient to edit a gene in a cell. The pharmaceutical compositions can be formulated for medical and/or veterinary use.

### 6.8. Methods of Altering a Cell

The disclosure further provides methods of using the Type II Cas proteins, gRNAs, nucleic acids (including pluralities of nucleic acids), systems, and particles (including pluralities of particles) of the disclosure for altering cells.

In one aspect, a method of altering a cell comprises contacting a eukaryotic cell (e.g., a human cell) with a nucleic acid, particle, system or pharmaceutical composition described herein.

Contacting a cell with a disclosed nucleic acid, particle, system or pharmaceutical composition can be achieved by any method known in the art and can be performed in vivo, ex vivo, or in vitro. In some embodiments, the methods can include obtaining one or more cells from a subject prior to contacting the cell(s) with a herein disclosed nucleic acid, particle, system or pharmaceutical composition. In some embodiments, the methods can further comprise returning or implanting the contacted cell or a progeny thereof to the subject.

Type II Cas and gRNA, as well as nucleic acids encoding Type II Cas and gRNAs can be delivered to a cell by any means known in the art, for example, by viral or non-viral delivery vehicles, electroporation or lipid nanoparticles.

A polynucleotide encoding Type II Cas and a gRNA, can be delivered to a cell (ex vivo or in vivo) by a lipid nanoparticle (LNP). LNPs can have, for example, a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm. LNPs can be made from cationic, anionic, neutral lipids, and combinations thereof. Neutral lipids, such as the fusogenic phospholipid DOPE or the membrane component cholesterol, can be included in LNPs as ‘helper lipids’ to enhance transfection activity and nanoparticle stability.

LNPs can also be comprised of hydrophobic lipids, hydrophilic lipids, or both hydrophobic and hydrophilic lipids. Lipids and combinations of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerCl4, and PEG-CerC20. Lipids can be combined in any number of molar ratios to produce a LNP. In addition, the polynucleotide(s) can be combined with lipid(s) in a wide range of molar ratios to produce a LNP.

Type II Cas and/or gRNAs can be delivered to a cell via an adeno-associated viral vector (e.g., of an AAV2, AAV5, AAV7m8, AAV8, AAV9, AAVrh8r, or AAVrh10 serotype), or by another viral vector. Other viral vectors include, but are not limited to lentivirus, adenovirus, alphavirus, enterovirus, pestivirus, baculovirus, herpesvirus, Epstein Barr virus, papovavirus, poxvirus, vaccinia virus, and herpes simplex virus. In some embodiments, a Type II Cas mRNA is formulated in a lipid nanoparticle, while a sgRNA is delivered to a cell in an AAV or other viral vector. In some embodiments, one or more AAV vectors (e.g., one or more AAV2, AAV5, AAV7m8, AAV8, AAV9, AAVrh8r, or AAVrh10 serotype) are used to deliver both a sgRNA and a Type II Cas. In some embodiments, a Type II Cas and a sgRNA are delivered using separate vectors. In other embodiments, a Type II Cas and a sgRNA are delivered using a single vector. BNK Type II Cas and AIK Type II Cas, with their relatively small size, can be delivered with a gRNA (e.g., sgRNA) using a single AAV vector.

Compositions and methods for delivering Type II Cas and gRNAs to a cell and/or subject are further described in PCT Patent Application Publications WO 2019/102381, WO 2020/012335, and WO 2020/053224, each of which is incorporated by reference herein in its entirety.

DNA cleavage can result in a single-strand break (SSB) or double-strand break (DSB) at particular locations within the DNA molecule. Such breaks can be and regularly are repaired by natural, endogenous cellular processes, such as homology-dependent repair (HDR) and non-homologous end-joining (NHEJ). These repair processes can edit the targeted polynucleotide by introducing a mutation, thereby resulting in a polynucleotide having a sequence which differs from the polynucleotide's sequence prior to cleavage by a Type II Cas.

NHEJ and HDR DNA repair processes consist of a family of alternative pathways. Non-homologous end-joining (NHEJ) refers to the natural, cellular process in which a double-stranded DNA-break is repaired by the direct joining of two non-homologous DNA segments. See, e.g. Cahill et al., 2006, Front. Biosci. 11:1958-1976. DNA repair by non-homologous end-joining is error-prone and frequently results in the untemplated addition or deletion of DNA sequences at the site of repair. Thus, NHEJ repair mechanisms can introduce mutations into the coding sequence which can disrupt gene function. NHEJ directly joins the DNA ends resulting from a double-strand break, sometimes with a modification of the polynucleotide sequence such as a loss of or addition of nucleotides in the polynucleotide sequence. The modification of the polynucleotide sequence can disrupt (or perhaps enhance) gene expression.

Homology-dependent repair (HDR) utilizes a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus.

A third repair mechanism includes microhomology-mediated end joining (MMEJ), also referred to as “Alternative NHEJ (ANHEJ)”, in which the genetic outcome is similar to NHEJ in that small deletions and insertions can occur at the cleavage site. MMEJ can make use of homologous sequences of a few base pairs flanking the DNA break site to drive a more favored DNA end joining repair outcome. In some instances, it may be possible to predict likely repair outcomes based on analysis of potential microhomologies at the site of the DNA break.

Modifications of a cleaved polynucleotide by HDR, NHEJ, and/or ANHEJ can result in, for example, mutations, deletions, alterations, integrations, gene correction, gene replacement, gene tagging, transgene insertion, nucleotide deletion, gene disruption, translocations and/or gene mutation. The aforementioned process outcomes are examples of editing a polynucleotide.

Advantages of ex vivo cell therapy approaches include the ability to conduct a comprehensive analysis of the therapeutic prior to administration. Nuclease-based therapeutics can have some level of off-target effects. Performing gene correction ex vivo allows a method user to characterize the corrected cell population prior to implantation, including identifying any undesirable off-target effects. Where undesirable effects are observed, a method user may opt not to implant the cells or cell progeny, may further edit the cells, or may select new cells for editing and analysis. Other advantages include ease of genetic correction in iPSCs compared to other primary cell sources. iPSCs are prolific, making it easy to obtain the large number of cells that will be required for a cell-based therapy. Furthermore, iPSCs are an ideal cell type for performing clonal isolations. This allows screening for the correct genomic correction, without risking a decrease in viability.

Although certain cells present an attractive target for ex vivo treatment and therapy, increased efficacy in delivery may permit direct in vivo delivery to such cells. Ideally the targeting and editing is directed to the relevant cells. Cleavage in other cells can also be prevented by the use of promoters only active in certain cell types and/or developmental stages.

Additional promoters are inducible, and therefore can be temporally controlled if the nuclease is delivered as a plasmid. The amount of time that delivered protein and RNA remain in the cell can also be adjusted using treatments or domains added to change the half-life. In vivo treatment would eliminate a number of treatment steps, but a lower rate of delivery can require higher rates of editing. In vivo treatment can eliminate problems and losses from ex vivo treatment and engraftment.

An advantage of in vivo gene therapy can be the ease of therapeutic production and administration. The same therapeutic approach and therapy has the potential to be used to treat more than one patient, for example a number of patients who share the same or similar genotype or allele. In contrast, ex vivo cell therapy typically requires using a subject's own cells, which are isolated, manipulated and returned to the same patient.

Progenitor cells (also referred to as stem cells herein) are capable of both proliferation and giving rise to more progenitor cells, which in turn have the ability to generate a large number of cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then to a cell with the capacity or potential, under particular circumstances, to differentiate to a more specialized or differentiated phenotype, and which retains the capacity, under certain circumstances, to proliferate without substantially differentiating. In one aspect, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell can derive from a multipotent cell that itself is derived from a multipotent cell, and so on. While each of these multipotent cells can be considered stem cells, the range of cell types that each can give rise to can vary considerably. Some differentiated cells also have the capacity to give rise to cells of greater developmental potential. Such capacity can be natural or can be induced artificially upon treatment with various factors. In many biological instances, stem cells can also be “multipotent” because they can produce progeny of more than one distinct cell type, but this is not required.

Human cells described herein can be induced pluripotent stem cells (iPSCs). An advantage of using iPSCs in the methods of the disclosure is that the cells can be derived from the same subject to which the progenitor cells are to be administered. That is, a somatic cell can be obtained from a subject, reprogrammed to an induced pluripotent stem cell, and then differentiated into a progenitor cell to be administered to the subject (e.g., an autologous cell). Because progenitors are essentially derived from an autologous source, the risk of engraftment rejection or allergic response can be reduced compared to the use of cells from another subject or group of subjects. In addition, the use of iPSCs negates the need for cells obtained from an embryonic source. Thus, in one aspect, the stem cells used in the disclosed methods are not embryonic stem cells.

Methods are known in the art that can be used to generate pluripotent stem cells from somatic cells. Pluripotent stem cells generated by such methods can be used in the method of the disclosure.

Reprogramming methodologies for generating pluripotent cells using defined combinations of transcription factors have been described. Mouse somatic cells can be converted to ES cell-like cells with expanded developmental potential by the direct transduction of Oct4, Sox2, Klf4, and c-Myc; see, e.g., Takahashi and Yamanaka, 2006, Cell 126(4): 663-76. iPSCs resemble ES cells, as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission (see, e.g., Maherali and Hochedlinger, 2008, Cell Stem Cell. 3(6):595-605), and tetraploid complementation.

Human iPSCs can be obtained using similar transduction methods, and the transcription factor trio, OCT4, SOX2, and NANOG, has been established as the core set of transcription factors that govern pluripotency; see, e.g., 2014, Budniatzky and Gepstein, Stem Cells Transl Med. 3(4):448-57; Barrett et al, 2014, Stem Cells Trans Med 3: 1-6 sctm.2014-0121; Focosi et al, 2014, Blood Cancer Journal 4: e211. The production of iPSCs can be achieved by the introduction of nucleic acid sequences encoding stem cell-associated genes into an adult, somatic cell, historically using viral vectors.

iPSCs can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., 2010, Cell Stem Cell, 7(5):618-30. Reprogramming can be achieved by introducing a combination of nucleic acids encoding stem cell-associated genes, including, for example, Oct-4 (also known as Oct-3/4 or Pouf5l), Soxl, Sox2, Sox3, Sox 15, Sox 18, NANOG, Klfl, Klf2, Klf4, Klf5, NR5A2, c-Myc, 1-Myc, n-Myc, Rem2, Tert, and LIN28. Reprogramming using the methods and compositions described herein can further comprise introducing one or more of Oct-3/4, a member of the Sox family, a member of the Klf family, and a member of the Myc family to a somatic cell. The methods and compositions described herein can further comprise introducing one or more of each of Oct-4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one aspect the reprogramming is not affected by a method that alters the genome. Thus, in such examples, reprogramming can be achieved, e.g., without the use of viral or plasmid vectors.

Efficiency of reprogramming (the number of reprogrammed cells) derived from a population of starting cells can be enhanced by the addition of various agents, e.g., small molecules, as shown by Shi et al., 2008, Cell-Stem Cell 2:525-528; Huangfu et al., 2008, Nature Biotechnology 26(7):795-797; and Marson et al., 2008, Cell-Stem Cell 3: 132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HD AC) inhibitors, valproic acid, 5′-azacytidine, dexamethasone, suberoylanilide, hydroxamic acid (SAHA), vitamin C, and trichostatin (TSA), among others. Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-IH,3H-benzo[de]isoquinolin-2-yl)-N-hydroxybutanamide), Phenylbutyrate (e.g., sodium phenylbutyrate) and Valproic Acid ((VP A) and other short chain fatty acids), Scriptaid, Suramin Sodium, Trichostatin A (TSA), APHA Compound 8, Apicidin, Sodium Butyrate, pi valoyloxy methyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-C1-UCHA (e.g., 6-(3-chlorophenylureido) caproic hydroxamic acid), AOE (2-amino-8-oxo-9, 10-epoxy decanoic acid), CHAP31 and CHAP 50. Other reprogramming enhancing agents include, for example, dominant negative forms of the HDACs (e.g, catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Titan Pharmaceuticals, MethylGene, and Sigma Aldrich.

To confirm the induction of pluripotent stem cells, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbxl5, Ecatl, Esgl, Eras, Gdfi, Fgf4, Cripto, Daxl, Zpf296, Slc2a3, Rexl, Utfl, and Natl. In one case, for example, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. Detection can involve not only RT-PCR, but also detection of protein markers. Intracellular markers can be best identified via RT-PCR, or protein detection methods such as immunocytochemistry, while cell surface markers are readily identified, e.g., by immunocytochemistry.

Pluripotency of isolated cells can be confirmed by tests evaluating the ability of the iPSCs to differentiate into cells of each of the three germ layers. As one example, teratoma formation in nude mice can be used to evaluate the pluripotent character of the isolated clones. The cells can be introduced into nude mice and histology and/or immunohistochemistry can be performed on a tumor arising from the cells. The growth of a tumor comprising cells from all three germ layers, for example, further indicates that the cells are pluripotent stem cells.

Patient-specific iPS cells or cell line can be created. There are many established methods in the art for creating patient specific iPS cells, e.g., as described in Takahashi and Yamanaka 2006; Takahashi, Tanabe et al. 2007. For example, the creating step can comprise: a) isolating a somatic cell, such as a skin cell or fibroblast, from the patient; and b) introducing a set of pluripotency-associated genes into the somatic cell in order to induce the cell to become a pluripotent stem cell. The set of pluripotency-associated genes can be one or more of the genes selected from the group consisting of OCT4, SOX1, SOX2, SOX3, SOX15, SOX18, NANOG, KLF1, KLF2, KLF4, KLF5, c-MYC, n-MYC, REM2, TERT and LIN28.

In some aspects, a biopsy or aspirate of a subject's bone marrow can be performed. A biopsy or aspirate is a sample of tissue or fluid taken from the body. There are many different kinds of biopsies or aspirates. Nearly all of them involve using a sharp tool to remove a small amount of tissue. If the biopsy will be on the skin or other sensitive area, numbing medicine can be applied first. A biopsy or aspirate can be performed according to any of the known methods in the art. For example, in a bone marrow aspirate, a large needle is used to enter the pelvis bone to collect bone marrow.

In some aspects, a mesenchymal stem cell can be isolated from a subject. Mesenchymal stem cells can be isolated according to any method known in the art, such as from a subject's bone marrow or peripheral blood. For example, marrow aspirate can be collected into a syringe with heparin. Cells can be washed and centrifuged on a Percoll™ density gradient. Cells, such as blood cells, liver cells, interstitial cells, macrophages, mast cells, and thymocytes, can be separated using density gradient centrifugation media, Percoll™. The cells can then be cultured in Dulbecco's modified Eagle's medium (DMEM) (low glucose) containing 10% fetal bovine serum (FBS) (Pittinger et. al., 1999, Science 284: 143-147).

### 6.8.1. Exemplary Genomic Targets

The Type II Cas proteins and gRNAs of the disclosure can be used to alter various genomic targets. In some aspects, the methods of altering a cell are methods for altering a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence. In some aspects, the methods of altering a cell are methods of altering a RHO genomic sequence. In some aspects, the methods of altering a cell are methods of altering a TRAC, B2M, PD1, or LAG3 genomic sequence. Reference genomic sequences are available in public databases, for example those maintained by NCBI. For example, DNMT1 has the NCBI gene ID 1786; RHO has the NCBI gene ID: 6010; TRAC has the NCBI gene ID:28755; B2M has the NCBI gene ID: 567; PD1 has the NCBI gene ID:5133; and LAG3 has the NCBI gene ID: 3902.

In some embodiments, the methods of altering a cell are methods for altering a hemoglobin subunit beta (HBB) gene. HBB mutations are associated with β-thalassemia and SCD. Dever et al., 2016 Nature 539(7629):384-389.

In some embodiments, the methods of altering a cell are methods for altering a CCR5 gene. CCR5 has demonstrated involvement in several different disease states including, but not limited to, human immunodeficiency virus (HIV) and acquired immune deficiency syndrome (AIDS). WO 2018/119359 describes CCR5 editing by CRISPR-Cas to make loss of function CCR5 in order to provide protection against HIV infection, decrease one or more symptoms of HIV infection, halt or delay progression of HIV to AIDS, and/or decrease one or more symptoms of AIDS.

In some embodiments, the methods of altering a cell are methods for altering a PD1, B2M gene, TRAC gene, or a combination thereof. CAR-T cells having PD1, B2M and TRAC genes disrupted by CRISPR-Type II Cas have demonstrated enhanced activity in preclinical glioma models. Choi et al., 2019, Journal for Immuno Therapy of Cancer 7:309.

In some embodiments, the methods of altering a cell are methods for altering an USH2A gene. Mutations in the USH2A gene can cause Usher syndrome type 2A, which is characterized by progressive hearing and vision loss.

In some embodiments, the methods of altering a cell are methods for altering a RHO gene. Mutations in the RHO gene can cause retinitis pigmentosa (RP).

Allele specific editing of human RHO alleles having pathogenic mutations (e.g., a P23 mutation such as P23H or a P347 mutation such as P347L, P347S, P347R, P347Q, P347T, or P347A) can be achieved using guide RNA (gRNA) molecules targeting the rs7984 SNP (for example having spacers as shown in Table 6) located in the 5′ untranslated region (UTR) of the RHO gene. SNPs are very common in the human population, and a significant proportion of subjects are heterozygous for the rs7984 SNP. For a subject heterozygous for the rs7984 SNP and heterozygous for a pathogenic RHO gene mutation, allele specific editing of the RHO allele having the pathogenic mutation can be achieved through the use of a gRNA targeting the SNP variant found in the subject's RHO allele having the pathogenic mutation. This allele-specific editing strategy, which does not directly target a specific pathogenic RHO gene mutation, advantageously allows editing of RHO genes having a variety of different pathogenic mutations. A rs7984 SNP targeting gRNA of the disclosure can be used in combination with a second gRNA targeting a second site in the RHO gene, for example a site in intron 1, to promote two cuts in the RHO gene having the pathogenic mutation. Cleaving the RHO gene having the pathogenic mutation at two sites can promote a deletion in the RHO gene having the pathogenic mutation, which can result in reduced mutant RHO protein expression.

Editing a subject's RHO allele can comprise editing a RHO allele in one or more cells from the subject (e.g., photoreceptor cells or retinal progenitor cells) or one or more cells derived from a cell of the subject (e.g., an induced pluripotent stem cell (iPSC)). For example, one or more cells from the subject or one or more cells derived from a cell of the subject can be contacted with a nucleic acid, system, or particle of the disclosure ex vivo, and cells having an edited RHO gene or progeny thereof can subsequently be implanted into the subject. Edited iPSCs can be differentiated, for instance into photoreceptor cells or retinal progenitor cells. In some embodiments, resultant differentiated cells can be implanted into the subject. When differentiated cells from the subject are edited, implantation of edited cells can proceed without an intervening differentiation step.

An in vivo method of RHO allele editing can comprise editing a RHO allele having a pathogenic mutation in a cell of a subject, such as photoreceptor cells or retinal progenitor cells. In some embodiments, the in vivo methods comprise administering one or more pharmaceutical compositions of the disclosure to or near the eye of a subject, e.g., by sub-retinal injection or intravitreal injection. For example, a single pharmaceutical composition comprising one or more AAV particles encoding one or more gRNAs (e.g., a gRNA targeting the rs7984 SNP and a gRNA targeting RHO intron 1) and a Type II Cas protein of the disclosure can be used; or alternatively, multiple pharmaceutical compositions can be used, for example a first pharmaceutical composition comprising an AAV particle encoding the gRNA(s) and a second, separate pharmaceutical composition comprising a second AAV particle encoding the Type II Cas protein. When multiple pharmaceutical compositions are used, they are preferably administered sufficiently close in time so that the gRNA(s) and Type II Cas protein provided by the pharmaceutical compositions are present together in vivo.

Targeting of (one or more of) human TRAC, human B2M, human PD1, and human LAG3 genes can be used, for example, in the engineering of chimeric antigen receptor (CAR) T cells. For example, CRISPR/Cas technology has been used to deliver CAR-encoding DNA sequences to loci such as TRAC and PD1 (see, e.g., Eyquem et al., 2017, Nature 543(7643):113-117; Hu et al., 2023, eClinicalMedicine 60:102010), while TRAC, B2M, PD1, and LAG3 knockout CAR T-cells have been reported (see, e.g., Dimitri et al., 2022, Molecular Cancer 21:78; Liu et al., 2016, Cell Research 27:154-157; Ren et al., 2017, Clin Cancer Res. 23(9):2255-2266; Zhang et al., 2017, Front Med. 11(4):554-562). Thus, the Type II Cas proteins and TRAC, B2M, PD1, and LAG3 guides of the disclosure can be used for targeted knock-in of an exogenous DNA sequence to a desired genomic site in a human cell and/or knock-out of TRAC, B2M, PD1, or LAG3 in a human cell, for example a human T cell. In some embodiments, T cells are edited ex vivo to produce CAR-T cells and subsequently administered to a subject in need of CAR-T cell therapy.

In some embodiments, the methods of altering a cell are methods for altering a DNMT1 gene. Mutations in the DNMT1 gene can cause DNMT1-related disorder, which is a degenerative disorder of the central and peripheral nervous systems. DNMT1-related disorder is characterized by sensory impairment, loss of sweating, dementia, and hearing loss.

### 7. EXAMPLES

### 7.1. Example 1: Identification and Characterization of Type II Cas Proteins

This Example describes studies performed to identify and characterize AHZW, ABSE, AIXM, AXTQ, AIWM, AIWR, AIYQ, EQSC, BDLP, and BDKL Type II Cas proteins.

### 7.1.1. Materials and Methods

### 7.1.1.1. Identification of Type II Cas Proteins From Metagenomic Data

154,723 bacterial and archaeal metagenome-assembled genomes (MAGs) reconstructed from the human microbiome (Pasolli, et al., 2019, Cell 176(3):649-662.e20) were screened in order to find new Type II Cas proteins. cas1, cas2 and cas9 genes were identified from the protein annotation, performed with Prokka version 1.12 (Seemann, 2014, Bioinformatics 30(14):2068-2069). CRISPR arrays were identified using MinCED version 0.4.2 (with default parameters) (Bland, et al., 2007, BMC bioinformatics 8:209). Only loci having a CRISPR array and cas1-2-9 genes at a maximum distance of 10 kbp from each other were considered. Loci containing Type II Cas proteins shorter than 950 aa were discarded. The resulting 17173 CRISPR-Type II Cas loci were filtered by selecting short proteins (less than 1100 aa) from putative unknown species. Type II Cas proteins from the same species, having similar length but slightly different sequence, were compared by multiple sequence alignment. Proteins presenting deletions in nucleasic domains were discarded. The remaining proteins were compared for sequencing coverage and the ortholog with the highest coverage was selected for each species.

7.1.1.2. tracrRNA Identification

Identification of tracrRNAs for CRISPR-Type II Cas loci of interest was performed with a method based on a work by Chyou and Brown (Chyou and Brown, 2019, RNA biology 16(4):423-434). Starting from unique direct repeats in the CRISPR array, BLAST version 2.2.31 (with parameters-task blastn-short-gapopen 2-gapextend 1-penalty-1-reward 1-evalue 1-word_size 8) (Altschul, et al., 1990, Journal of Molecular Biology 215(3):403-410) was used to identify anti-repeats within a 3000 bp window flanking the CRISPR-Type II Cas locus. A custom version of RNIE (Gardner, et al., 2011, Nucleic Acids Research 39(14):5845-5852) was used to predict Rho-independent transcription terminators (RITs) near anti-repeats. Putative tracrRNA sequences, starting with an anti-repeat and ending with either a RIT (when found) or a poly-T, were combined with directed repeats to form sgRNA scaffolds. The secondary structure of sgRNA scaffolds was predicted using RNAsubopt version 2.4.14 (with parameters--noLP-e 5) (Lorenz, et al., 2011, Algorithms for Molecular Biology 6(1):26). sgRNAs lacking the functional modules identified by (Briner, et al., 2014 Molecular Cell 56(2):333-339), namely the repeat:anti-repeat duplex, nexus and 3′ hairpin-like folds, were discarded.

### 7.1.2. Results

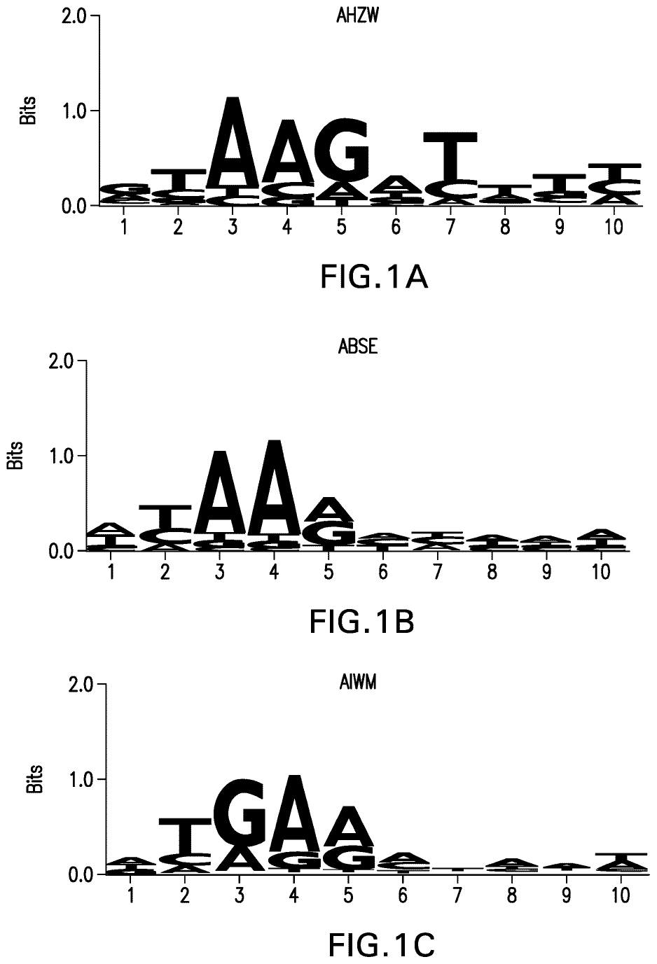

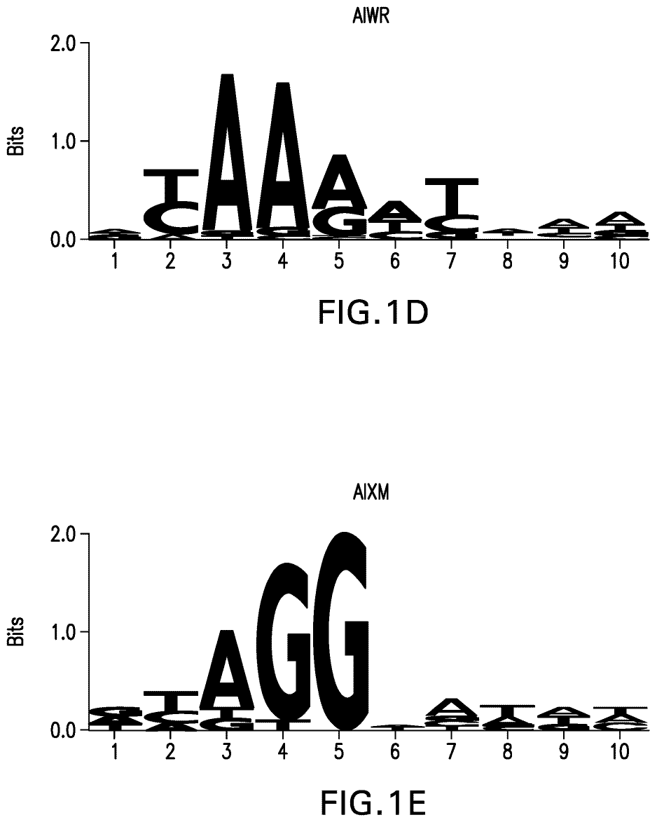

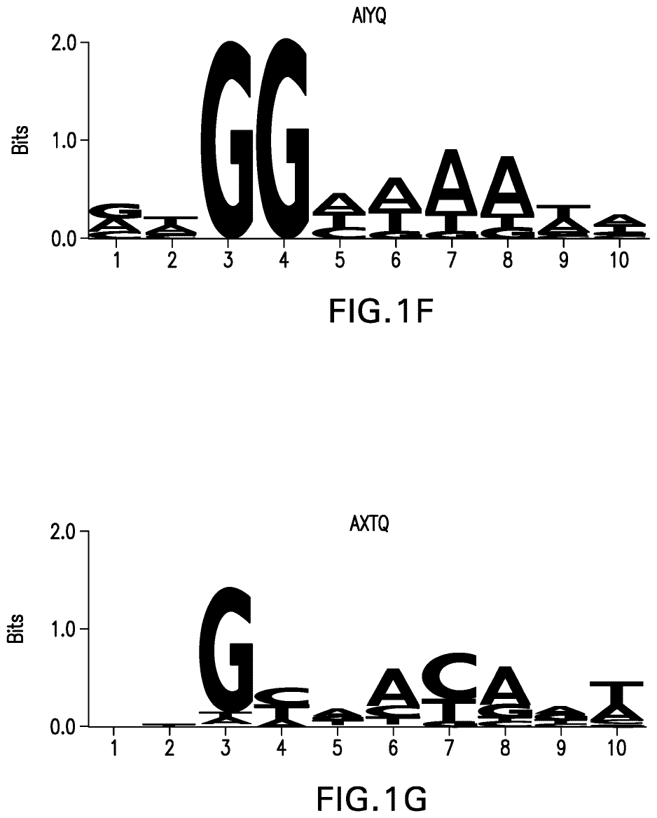

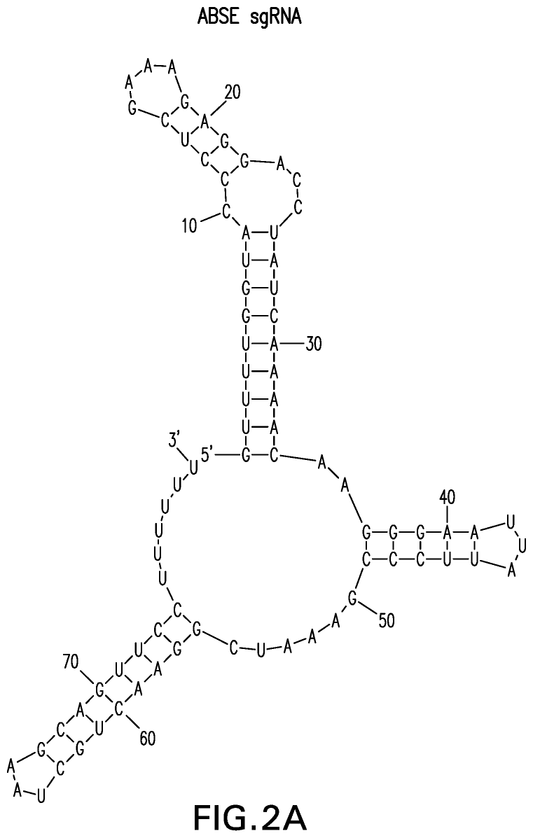

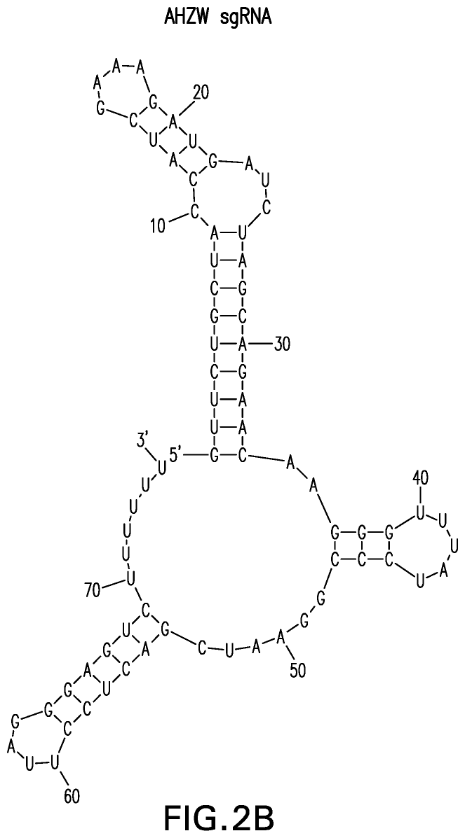

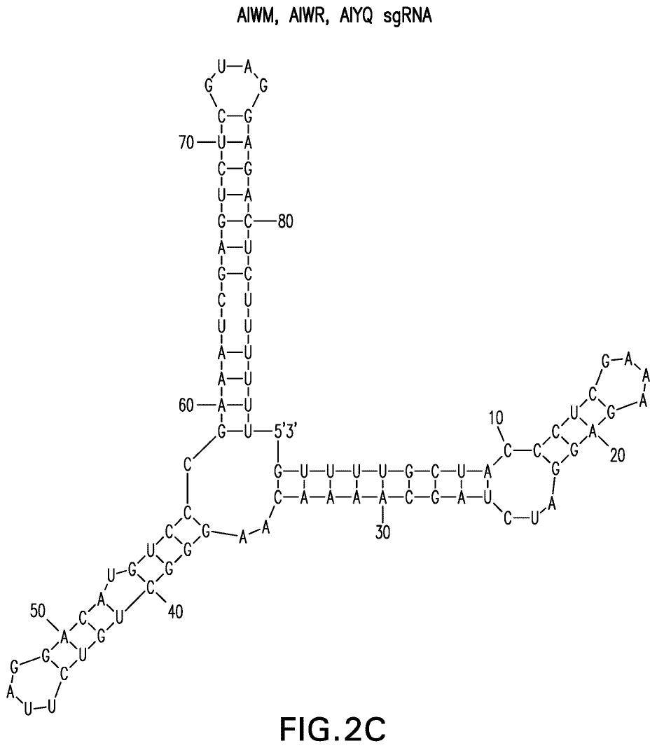

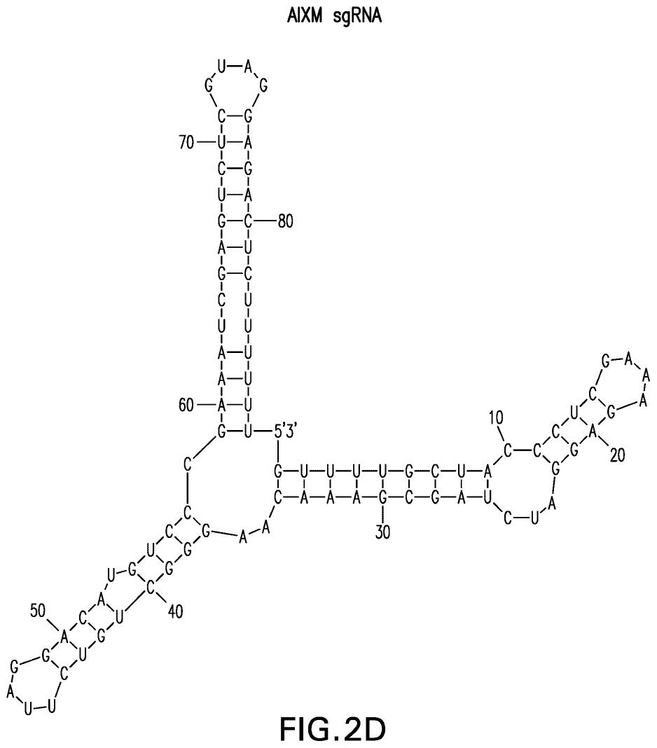

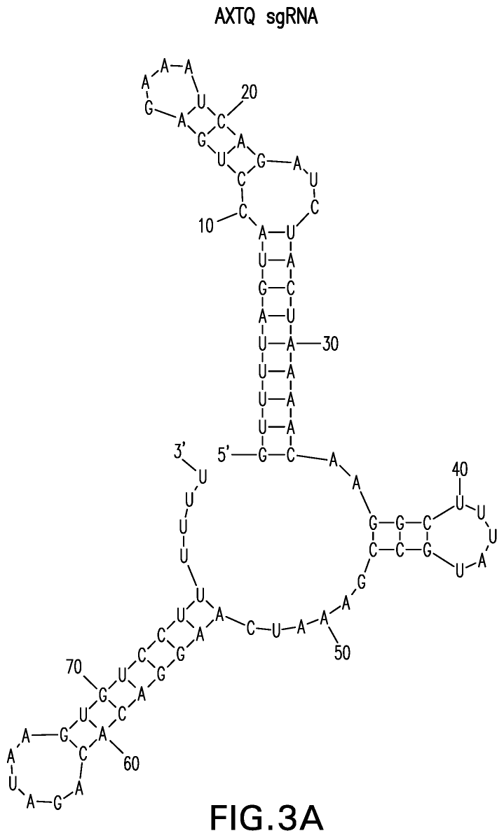

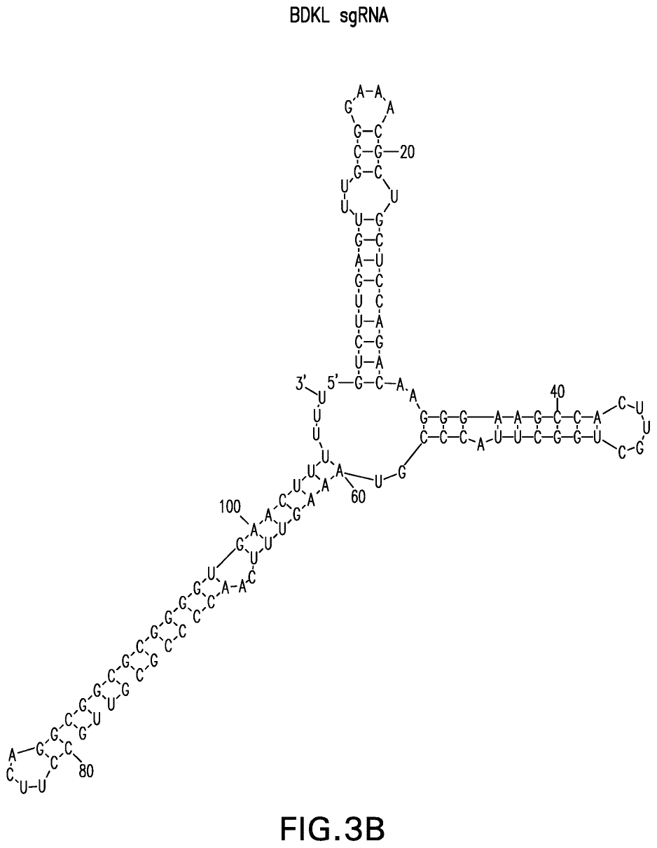

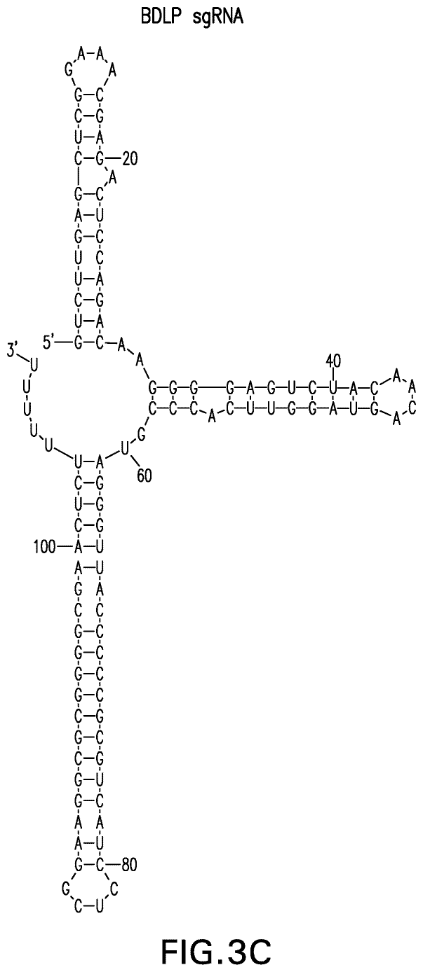

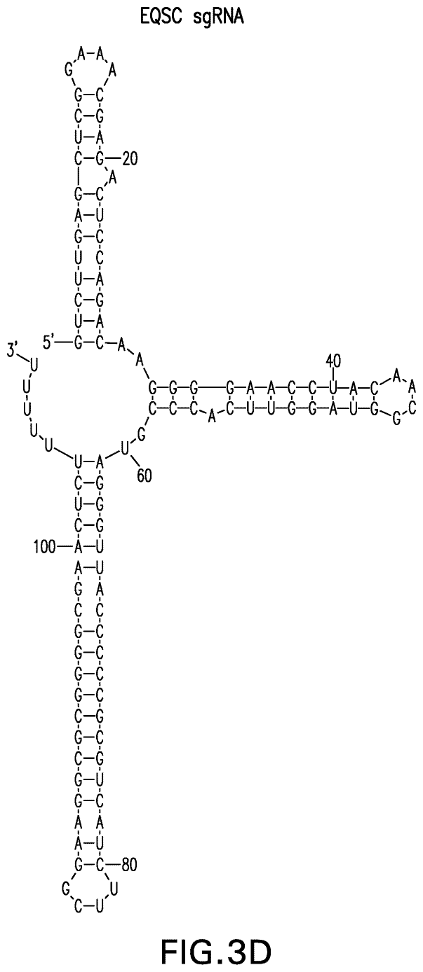

AHZW, ABSE, AIXM, AXTQ, AIWM, AIWR, AIYQ, EQSC, BDLP, and BDKL Type II Cas proteins were identified. Amino acid sequences of AHZW, ABSE, AIXM, AXTQ, AIWM, AIWR, AIYQ, EQSC, BDLP, and BDKL Type II Cas proteins and nucleotide sequences encoding exemplary AHZW, ABSE, AIXM, AXTQ, AIWM, AIWR, AIYQ, EQSC, BDLP, and BDKL Type II Cas proteins are shown in Tables 1A-2C. Exemplary predicted PAM sequences are shown in Table 5. Exemplary predicted PAM logos are shown in FIGS. 1A-1G. crRNA and tracrRNA for the nucleases are described in Section 6.3. Exemplary sgRNA scaffolds are shown in Tables 7A-7B. Schematic representations of the hairpin structure of exemplary Type II Cas protein sgRNAs are shown in FIGS. 2A-3D and FIG. 8.

### 7.2. Example 2: Further Characterization of Type II Cas Proteins

This Example describes studies performed to further characterize AHZW, ABSE, AIXM, AXTQ, AIWM, AIWR, AIYQ, EQSC, BDLP, and BDKL Type II Cas proteins.

### 7.2.1. Materials and Methods

### 7.2.1.1. In Vitro Type II PAM Identification Assay

In vitro PAM evaluation of the Type II Cas proteins was performed according to the protocol from Karvelis, Young and Siksnys (Karvelis et al., 2019, Methods in Enzymology 616:219-240). In brief: a human codon optimized version of each Type II Cas protein gene (see Tables 1A-1G and 2A-2C for sequences) obtained as a synthetic construct (Twist Bioscience) was cloned into an expression vector for in vitro transcription and translation (IVT) (pT7-N-His-GST, Thermo Fisher Scientific). The sgRNAs to perform the assay were obtained by in vitro transcription of the guide using the High Yield T7 RNA Synthesis Kit (Jena Bioscience) starting from a PCR template generated by amplification from each sgRNA expression construct. The primers used to generate the IVT templates are reported in Table 8. In vitro transcribed gRNAs were subsequently purified using the MEGAClear™ Transcription Clean-up kit (Thermo Fisher Scientific). The in vitro transcription and translation reaction for Type II Cas expression was performed according to the manufacturer's protocol (1-Step Human High-Yield Mini IVT Kit, Thermo Fisher Scientific). The nuclease-guide RNA RNP complex was assembled by combining 20 μL of the supernatant containing the soluble Type II Cas protein with 1 μL of RiboLock™ RNase Inhibitor (Thermo Fisher Scientific) and 2 μg of guide RNA (previously transcribed in vitro). The RNP complex was used to digest 1 μg of a PAM plasmid DNA library (containing a defined target sequence flanked at the 3′-end by a randomized 8 nucleotide PAM sequence) for 1 hour at 37° C.

TABLE 8
Sequences of the primers used for PCR amplification of gRNAs used as templates
for in vitro transcription

| Primer name | Sequence (5′→3′) | SEQ ID NO |
|---|---|---|
| IVT-T7-ABSE-gRNA-F | cctcTAATACGACTCACTATAGGtcGTCGCCCTCGAACTTC | 131 |
| | ACCTGTTTTGGTACCCTCGAAAGAG | |
| IVT-ABSE-gRNA-R | AAAAAAGGAACTGCTTAGCAGTT | 132 |
| IVT-T7-AHZW-gRNA-F | cctcTAATACGACTCACTATAGGtcGTCGCCCTCGAACTTC | 133 |
| | ACCTGTTCTGCTACCATCGAAAGAT | |
| IVT-AHZW-gRNA-R | AAAAAAGACTCCCTAAGGAGTC | 134 |
| IVT- | TAATACGACTCACTATAGGTCGTCGCCCTCGAACTTCA | 135 |
| AIWM_AIWR_AIYQ- | CCTGTTTTGCTACCCTCGAAAG | |
| gRNA-F | | |
| IVT- | TAATACGACTCACTATAGGTCGTCGCCCTCGAACTTCA | 136 |
| AIWM_AIWR_AIYQ- | CCTGTTTTGCTACCCTCACAATTTTTG | |
| gRNA full-length-F | | |
| IVT- | AAAAAGAGTCTCCTACGAGAC | 137 |
| AIWM_AIWR_AIYQ- | | |
| gRNA-R | | |
| IVT-T7-AIXM-gRNA-F | cctcTAATACGACTCACTATAGGtcGTCGCCCTCGAACTTC | 138 |
| | ACCTGTTTTGCTACCCTCGAAAGAG | |
| IVT-AIXM-gRNA-R | AAAAAAGAGTCTCCTACGAGAC | 139 |
| IVT-T7-AXTQ-gRNA-F | cctcTAATACGACTCACTATAGGtcGTCGCCCTCGAACTTC | 140 |
| | ACCTGTTTTAGTACCTGAGAAATCAG | |
| IVT-AXTQ-gRNA-R | AAAAAAGGACACTTATCTGTGTC | 141 |
| IVT-T7-BDKL-gRNA-F | cctcTAATACGACTCACTATAGGtcGTCGCCCTCGAACTTC | 142 |
| | ACCTGTCTTGAGTTTGCGGAAACG | |
| IVT-BDKL-gRNA-R | AAAAAAGTTCACCCCGCGC | 143 |
| IVT-T7-BDLP-gRNA-F | cctcTAATACGACTCACTATAGGtcGTCGCCCTCGAACTTC | 144 |
| | ACCTGTCTTGAGCTCGGAAACGA | |
| IVT-BDLP-gRNA-R | AAAAAAGAGTTCGCCCCGC | 145 |
| IVT-T7-EQSC-gRNA-F | cctcTAATACGACTCACTATAGGtcGTCGCCCTCGAACTTC | 144 |
| | ACCTGTCTTGAGCTCGGAAACGA | |
| IVT-EQSC-gRNA-R | AAAAAAGAGTTCGCCCCGC | 145 |

A double stranded DNA adapter (Table 9) was ligated to the DNA ends generated by the targeted Type II Cas cleavage and the final ligation product was purified using a GeneJet™ PCR Purification Kit (Thermo Fisher Scientific).

TABLE 9
Sequences of the two oligonucleotides used to
prepare the dsDNA adapter for the in vitro PAM
assay

| Oligo | Sequence (5′→3) |
|---|---|
| Oligo UP | CGGCATTCCTGCTGAACCGCTCTTCCGATCT |
| | (SEQ ID NO: 146) |
| Oligo BOTTOM | GATCGGAAGAGCGGTTCAGCAGGAATGCCG |
| | (SEQ ID NO: 147) |

One round of a two-step PCR (Phusion® HF DNA polymerase, Thermo Fisher Scientific) was performed to enrich the sequences that were cut using a set of forward primers annealing on the adapter and a reverse primer designed on the plasmid backbone downstream of the PAM (Table 10). A second round of PCR was performed to attach the Illumina indexes and adapters. PCR products were purified using the GeneJet™ PCR Purification Kit (Thermo Fisher Scientific).

TABLE 10
Sequences of the primers used for NGS library preparation in the in vitro PAM assay

| Primer name | Sequence (5′→3′) | SEQ ID NO |
|---|---|---|
| F4a | TCGTCGGCAGCGTCAGATGTGTATAAGAGACAGCTGCTGAACCGCTC | 148 |
| | TTCCGATC | |
| F4b | TCGTCGGCAGCGTCAGATGTGTATAAGAGACAGTAAGACTGCTGAACC | 149 |
| | GCTCTTCCGATC | |
| F4c | TCGTCGGCAGCGTCAGATGTGTATAAGAGACAGGCTAGACCTAATGTG | 150 |
| | ATCTGCTGAACCGCTCTTCCGATC | |
| R3 | GTCTCGTGGGCTCGGAGATGTGTATAAGAGACAGTCTGCGTTCTGATT | 151 |
| | TAATCTGTATCAGGC | |

The library was analyzed with a 71-bp single read sequencing, using a flow cell v2 micro, on an Illumina MiSeq™ sequencer.

PAM sequences were extracted from Illumina MiSeq™ reads and used to generate PAM sequence logos, using Logomaker version 0.8. PAM heatmaps were used to display PAM enrichment, computed dividing the frequency of PAM sequences in the cleaved library by the frequency of the same sequences in a control uncleaved library.

### 7.2.1.2. Plasmids

Type II Cas proteins were expressed in mammalian cells from a plasmid vector characterized by an EF1-alpha-driven cassette. Each Type II Cas protein coding sequence was human codon-optimized and modified by the addition of an SV5 tag at the N-terminus and two bipartite nuclear localization signals (one at the N-terminus and one at the C-terminus) (sequences are shown in Tables 1A-1G and 2A-2C). sgRNAs were expressed from a U6-driven cassette located on an independent plasmid construct. The human codon-optimized coding sequences of the Type II Cas proteins, as well as the sgRNA scaffolds, were obtained by synthesis from Twist Bioscience. Spacer sequences were cloned into the sgRNA plasmid as annealed DNA oligonucleotides (Eurofins Genomics) using a double Bsal site present in the plasmid. The list of spacer sequences and relative cloning oligonucleotides used in the present example is reported in Table 11. In all cases in which a spacer did not contain a matching native 5′-G, this nucleotide was appended upstream the targeting sequence in order to allow efficient transcription from a U6 promoter.

TABLE 11
Spacer sequences and cloning oligos

| | | | | | Oligo used to clone the spacer in | | | |
|---|---|---|---|---|---|---|---|---|
| Spacer sequences used in the EGFP reporter assay | | | | | sgRNA plasmid (**) | | | |
| | Proto- | SEQ ID | | SEQ ID | Oligo 1 | SEQ ID | Oligo 2 | SEQ ID |
| Name | spacer | NO | Target (*) | NO | (5′ > 3′) | NO | (5′→3′) | NO |
| gRNA- | GCTGGAC | 152 | cgaGCTGGACGGCG | 185 | caccGCT | 220 | aaacGGCC | 253 |
| ABSE- | GGCGACG | | ACGTAAACGGCCA | | GGACGG | | GTTTACG | |
| EGFP- | TAAACGG | | CAAGttc | | CGACGT | | TCGCCGT | |
| g1 | CC | | | | AAACGG | | CCAGC | |
| | | | | | CC | | | |
| gRNA- | GGGCGA | 153 | cgaGGGCGAGGGC | 186 | caccGGG | 221 | aaacCGTA | 254 |
| ABSE- | GGGCGAT | | GATGCCACCTACG | | CGAGGG | | GGTGGCA | |
| EGFP- | GCCACCT | | GCAAGctg | | CGATGC | | TCGCCCT | |
| g2 | ACG | | | | CACCTA | | CGCCC | |
| | | | | | CG | | | |
| gRNA- | CCTGAAG | 154 | gacCCTGAAGTTCA | 187 | caccGCC | 222 | aaacCGGT | 255 |
| ABSE- | TTCATCT | | TCTGCACCACCGG | | TGAAGT | | GGTGCAG | |
| EGFP- | GCACCAC | | CAAGctg | | TCATCT | | ATGAACT | |
| g3 | CG | | | | GCACCA | | TCAGGC | |
| | | | | | CCG | | | |
| gRNA- | GTCAGGG | 155 | tagGTCAGGGTGGT | 188 | caccGTC | 223 | gaacCCCA | 256 |
| AHZW- | TGGTCAC | | CACGAGGGTGGGC | | AGGGTG | | CCCTCGT | |
| EGFP- | GAGGGTG | | CAGGGCacg | | GTCACG | | GACCACC | |
| g1 | GG | | | | AGGGTG | | CTGAC | |
| | | | | | GG | | | |
| gRNA- | CCACATG | 156 | cgaCCACATGAAGC | 189 | caccGCC | 224 | gaacAGAA | 257 |
| AHZW- | AAGCAGC | | AGCACGACTTCTTC | | ACATGA | | GTCGTGC | |
| EGFP- | ACGACTT | | AAGTCcgc | | AGCAGC | | TGCTTCA | |
| g2 | CT | | | | ACGACT | | TGTGGC | |
| | | | | | TCT | | | |
| gRNA- | CTTCTTC | 157 | catCTTCTTCAAGGA | 190 | caccGCT | 225 | gaacAGTT | 258 |
| AHZW- | AAGGACG | | CGACGGCAACTAC | | TCTTCAA | | GCCGTCG | |
| EGFP- | ACGGCAA | | AAGACccg | | GGACGA | | TCCTTGA | |
| g3 | CT | | | | CGGCAA | | AGAAGC | |
| | | | | | CT | | | |
| gRNA- | GTTTACG | 158 | gccGTTTACGTCGC | 191 | caccGTTT | 226 | aaacTCGA | 259 |
| AIXM- | TCGCCGT | | CGTCCAGCTCGAC | | ACGTCG | | GCTGGAC | |
| EGFP- | CCAGCTC | | CAGGatg | | CCGTCC | | GGCGAC | |
| g1 | GA | | | | AGCTCG | | GTAAAC | |
| | | | | | A | | | |
| gRNA- | GTCAGGG | 155 | tagGTCAGGGTGGT | 192 | caccGTC | 223 | aaacCCCA | 260 |
| AIXM- | TGGTCAC | | CACGAGGGTGGGC | | AGGGTG | | CCCTCGT | |
| EGFP- | GAGGGTG | | CAGGgca | | GTCACG | | GACCACC | |
| g2 | GG | | | | AGGGTG | | CTGAC | |
| | | | | | GG | | | |
| gRNA- | TCCGCCA | 159 | aagTCCGCCATGCC | 193 | caccGTC | 227 | aaacACGT | 261 |
| AIXM- | TGCCCGA | | CGAAGGCTACGTC | | CGCCAT | | AGCCTTC | |
| EGFP- | AGGCTAC | | CAGGagc | | GCCCGA | | GGGCATG | |
| g3 | GT | | | | AGGCTA | | GCGGAC | |
| | | | | | CGT | | | |
| gRNA- | TTTACGT | 160 | ccgTTTACGTCGCC | 194 | caccGTTT | 228 | aaacGTCG | 262 |
| AIYQ- | CGCCGTC | | GTCCAGCTCGACC | | ACGTCG | | AGCTGGA | |
| EGFP- | CAGCTCG | | AGGATggg | | CCGTCC | | CGGCGAC | |
| g1 | AC | | | | AGCTCG | | GTAAAC | |
| | | | | | AC | | | |
| gRNA- | GACCCTG | 161 | gctGACCCTGAAGTT | 195 | caccGAC | 229 | aaacTGGT | 263 |
| AIYQ- | AAGTTCA | | CATCTGCACCACC | | CCTGAA | | GCAGATG | |
| EGFP- | TCTGCAC | | GGCAagc | | GTTCAT | | AACTTCA | |
| g2 | CA | | | | CTGCAC | | GGGTC | |
| | | | | | CA | | | |
| gRNA- | AAGTCGT | 162 | aagAAGTCGTGCTG | 196 | caccGAA | 230 | aaacGACC | 264 |
| AIYQ- | GCTGCTT | | CTTCATGTGGTCG | | GTCGTG | | ACATGAA | |
| EGFP- | CATGTGG | | GGGTAgcg | | CTGCTT | | GCAGCAC | |
| g3 | TC | | | | CATGTG | | GACTTC | |
| | | | | | GTC | | | |
| gRNA- | GGCAAGC | 163 | tacGGCAAGCTGAC | 197 | caccGGC | 231 | aaacATGA | 265 |
| AXTQ- | TGACCCT | | CCTGAAGTTCATCT | | AAGCTG | | ACTTCAG | |
| EGFP- | GAAGTTC | | GCACCAccg | | ACCCTG | | GGTCAGC | |
| g1 | AT | | | | AAGTTC | | TTGCC | |
| | | | | | AT | | | |
| gRNA- | GCACTGC | 164 | gaaGCACTGCACGC | 198 | caccGCA | 232 | aaacCCCT | 266 |
| AXTQ- | ACGCCGT | | CGTAGGTCAGGGT | | CTGCAC | | GACCTAC | |
| EGFP- | AGGTCAG | | GGTCACGagg | | GCCGTA | | GGCGTGC | |
| g2 | GG | | | | GGTCAG | | AGTGC | |
| | | | | | GG | | | |
| gRNA- | ATGCCCG | 165 | gccATGCCCGAAGG | 199 | caccGAT | 233 | aaacTCCT | 267 |
| AXTQ- | AAGGCTA | | CTACGTCCAGGAG | | GCCCGA | | GGACGTA | |
| EGFP- | CGTCCAG | | CGCACCAtct | | AGGCTA | | GCCTTCG | |
| g3 | GA | | | | CGTCCA | | GGCATC | |
| | | | | | GGA | | | |
| gRNA- | GCCCATC | 166 | ggtGCCCATCCTGG | 200 | caccGCC | 234 | agacCCGT | 268 |
| BDLP- | CTGGTCG | | TCGAGCTGGACGG | | CATCCT | | CCAGCTC | |
| EGFP- | AGCTGGA | | CGACGTaaa | | GGTCGA | | GACCAGG | |
| g1 | CGG | | | | GCTGGA | | ATGGGC | |
| | | | | | CGG | | | |
| gRNA- | GGGGTAG | 167 | gtcGGGGTAGCGGC | 201 | caccGGG | 235 | agacTGCA | 269 |
| BDLP- | CGGCTGA | | TGAAGCACTGCAC | | GTAGCG | | GTGCTTC | |
| EGFP- | AGCACTG | | GCCGTagg | | GCTGAA | | AGCCGCT | |
| g2 | CA | | | | GCACTG | | ACCCC | |
| | | | | | CA | | | |
| gRNA- | GTAGTTG | 168 | cttGTAGTTGCCGTC | 202 | caccGTA | 236 | agacTTCT | 270 |
| BDLP- | CCGTCGT | | GTCCTTGAAGAAG | | GTTGCC | | TCAAGGA | |
| EGFP- | CCTTGAA | | ATGGTgcg | | GTCGTC | | CGACGGC | |
| g3 | GAA | | | | CTTGAA | | AACTAC | |
| | | | | | GAA | | | |
| gRNA- | GGCGAG | 169 | aagGGCGAGGAGCT | 203 | caccGGC | 237 | agacACCC | 271 |
| BDKL- | GAGCTGT | | GTTCACCGGGGTG | | GAGGAG | | CGGTGAA | |
| EGFP- | TCACCGG | | GTGCCCAtcc | | CTGTTC | | CAGCTCC | |
| g1 | GGT | | | | ACCGGG | | TCGCC | |
| | | | | | GT | | | |
| gRNA- | CCTGGCC | 170 | tgcCCTGGCCCACC | 204 | caccGCC | 238 | agacGGTG | 272 |
| BDKL- | CACCCTC | | CTCGTGACCACCC | | TGGCCC | | GTCACGA | |
| EGFP- | GTGACCA | | TGACCTAcgg | | ACCCTC | | GGGTGG | |
| g2 | CC | | | | GTGACC | | GCCAGGC | |
| | | | | | ACC | | | |
| gRNA- | AGCACGA | 171 | agcAGCACGACTTC | 205 | caccGAG | 239 | agacGGC | 273 |
| BDKL- | CTTCTTC | | TTCAAGTCCGCCAT | | CACGAC | | GGACTTG | |
| EGFP- | AAGTCCG | | GCCCGAagg | | TTCTTCA | | AAGAAGT | |
| g3 | CC | | | | AGTCCG | | CGTGCTC | |
| | | | | | CC | | | |
| gRNA- | GCCCATC | 172 | ggtGCCCATCCTGG | 206 | caccGCC | 240 | agacCGTC | 274 |
| EQSC- | CTGGTCG | | TCGAGCTGGACGG | | CATCCT | | CAGCTCG | |
| EGFP- | AGCTGGA | | CGACGTAaac | | GGTCGA | | ACCAGGA | |
| g1 | CG | | | | GCTGGA | | TGGGC | |
| | | | | | CG | | | |
| gRNA- | TGCAGAT | 173 | tggTGCAGATGAACT | 207 | caccGTG | 241 | agacGCTG | 275 |
| EQSC- | GAACTTC | | TCAGGGTCAGCTT | | CAGATG | | ACCCTGA | |
| EGFP- | AGGGTCA | | GCCGTAggt | | AACTTCA | | AGTTCAT | |
| g2 | GC | | | | GGGTCA | | CTGCAC | |
| | | | | | GC | | | |
| gRNA- | GCACGAC | 174 | gcaGCACGACTTCT | 208 | caccGCA | 242 | agacTGGC | 276 |
| EQSC- | TTCTTCAA | | TCAAGTCCGCCAT | | CGACTT | | GGACTTG | |
| EGFP- | GTCCGCC | | GCCCGAAggc | | CTTCAA | | AAGAAGT | |
| g3 | A | | | | GTCCGC | | CGTGC | |
| | | | | | CA | | | |
| gRNA- | CGCCCTC | 175 | catCGCCCTCGCCC | 209 | caccGCG | 243 | aaacCGTG | 277 |
| AIWM- | GCCCTCG | | TCGCCGGACACGC | | CCCTCG | | TCCGGCG | |
| EGFP- | CCGGACA | | TGAActt | | CCCTCG | | AGGGCGA | |
| g1-23 | CG | | | | CCGGAC | | GGGCGC | |
| nt | | | | | ACG | | | |
| gRNA- | GCCCTCG | 176 | atcGCCCTCGCCCT | 210 | caccGCC | 244 | aaacCGTG | 278 |
| AIWM- | CCCTCGC | | CGCCGGACACGCT | | CTCGCC | | TCCGGCG | |
| EGFP- | CGGACAC | | GAActt | | CTCGCC | | AGGGCGA | |
| g1-22 | G | | | | GGACAC | | GGGC | |
| nt | | | | | G | | | |
| gRNA- | GCTTCAT | 177 | gctGCTTCATGTGGT | 211 | caccGCT | 245 | aaacCCGC | 279 |
| AIWM- | GTGGTCG | | CGGGGTAGCGGCT | | TCATGT | | TACCCCG | |
| EGFP- | GGGTAGC | | GAAgca | | GGTCGG | | ACCACAT | |
| g2-23nt | GG | | | | GGTAGC | | GAAGC | |
| | | | | | GG | | | |
| gRNA- | TTCATGT | 178 | tgcTTCATGTGGTCG | 212 | caccGTT | 246 | aaacCCGC | 280 |
| AIWM- | GGTCGGG | | GGGTAGCGGCTGA | | CATGTG | | TACCCCG | |
| EGFP- | GTAGCGG | | Agca | | GTCGGG | | ACCACAT | |
| g2-21 | | | | | GTAGCG | | GAAC | |
| nt | | | | | G | | | |
| gRNA- | GCAACTA | 179 | acgGCAACTACAAG | 213 | caccGCA | 247 | aaacCTCG | 281 |
| AIWM- | CAAGACC | | ACCCGCGCCGAGG | | ACTACA | | GCGCGG | |
| EGFP- | CGCGCCG | | TGAAgtt | | AGACCC | | GTCTTGT | |
| g3-23 | AG | | | | GCGCCG | | AGTTGC | |
| nt | | | | | AG | | | |
| gRNA- | AACTACA | 180 | ggcAACTACAAGAC | 214 | caccGAA | 248 | aaacCTCG | 282 |
| AIWM- | AGACCCG | | CCGCGCCGAGGTG | | CTACAA | | GCGCGG | |
| EGFP- | CGCCGAG | | AAgtt | | GACCCG | | GTCTTGT | |
| g3-21 | | | | | CGCCGA | | AGTTC | |
| nt | | | | | G | | | |
| gRNA- | GTTTACG | 158 | gccGTTTACGTCGC | 215 | caccGTTT | 226 | aaacTCGA | 259 |
| AIWR- | TCGCCGT | | CGTCCAGCTCGAC | | ACGTCG | | GCTGGAC | |
| EGFP- | CCAGCTC | | CAGGATggg | | CCGTCC | | GGCGAC | |
| g1-23 | GA | | | | AGCTCG | | GTAAAC | |
| nt | | | | | A | | | |
| gRNA- | TTACGTC | 181 | cgtTTACGTCGCCGT | 216 | caccGTT | 249 | aaacTCGA | 283 |
| AIWR- | GCCGTCC | | CCAGCTCGACCAG | | ACGTCG | | GCTGGAC | |
| EGFP- | AGCTCGA | | GATggg | | CCGTCC | | GGCGAC | |
| g1-21 | | | | | AGCTCG | | GTAAC | |
| nt | | | | | A | | | |
| gRNA- | GTAGCGG | 182 | gggGTAGCGGCTGA | 217 | caccGTA | 250 | aaacGGC | 284 |
| AIWR- | CTGAAGC | | AGCACTGCACGCC | | GCGGCT | | GTGCAGT | |
| EGFP- | ACTGCAC | | GTAGGTCagg | | GAAGCA | | GCTTCAG | |
| g2-24 | GCC | | | | CTGCAC | | CCGCTAC | |
| nt | | | | | GCC | | | |
| gRNA- | GCGGCTG | 183 | gtaGCGGCTGAAGC | 218 | caccGCG | 251 | aaacGGC | 285 |
| AIWR- | AAGCACT | | ACTGCACGCCGTA | | GCTGAA | | GTGCAGT | |
| EGFP- | GCACGCC | | GGTCagg | | GCACTG | | GCTTCAG | |
| g2-21 | | | | | CACGCC | | CCGC | |
| nt | | | | | | | | |
| gRNA- | GTCAGGG | 155 | tagGTCAGGGTGGT | 188 | caccGTC | 223 | aaacCCCA | 260 |
| AIWR- | TGGTCAC | | CACGAGGGTGGGC | | AGGGTG | | CCCTCGT | |
| EGFP- | GAGGGTG | | CAGGGCacg | | GTCACG | | GACCACC | |
| g3-23 | GG | | | | AGGGTG | | CTGAC | |
| nt | | | | | GG | | | |
| gRNA- | CAGGGTG | 184 | ggtCAGGGTGGTCA | 219 | caccGCA | 252 | aaacCCCA | 286 |
| AIWR- | GTCACGA | | CGAGGGTGGGCCA | | GGGTGG | | CCCTCGT | |
| EGFP- | GGGTGG | | GGGCacg | | TCACGA | | GACCACC | |
| g3-21 | G | | | | GGGTGG | | CTGC | |
| nt | | | | | G | | | |

(*) The target sequences are reported with three flanking nucleotides on each side. The PAM sequence is highlighted in bold.
(**) The cloning overhang is reported in lowercase. Nucleotides highlighted in bold represent 5′-G appended to favor transcription from canonical U6 Pol III promoters.

### 7.2.1.3. Cell Lines

U2OS-EGFP cells, harboring a single integrated copy of an EGFP reporter gene, were cultured in DMEM (Life Technologies) supplemented with 10% FBS (Life Technologies), 2 mM L-Glutamine (Life Technologies) and penicillin/streptomycin (Life Technologies). All cells were incubated at 37° C. and 5% CO2 in a humidified atmosphere. All cells tested mycoplasma negative (PlasmoTest, Invivogen).

### 7.2.1.4. Cell Line Transfections

To perform editing studies, 200,000 U2OS-EGFP cells were nucleofected with 500 ng of nuclease-expressing plasmid and 250 ng of sgRNA-expressing plasmid containing a guide designed to target EGFP using the 4D-Nucleofector™ SE Kit (Lonza), DN-100 program, according to the manufacturer's protocol. After electroporation, cells were plated in a 24-well plate. EGFP knock-out was analyzed 4 days after nucleofection using a BD FACSymphony™ A1 flow cytometer.

### 7.2.2. Results

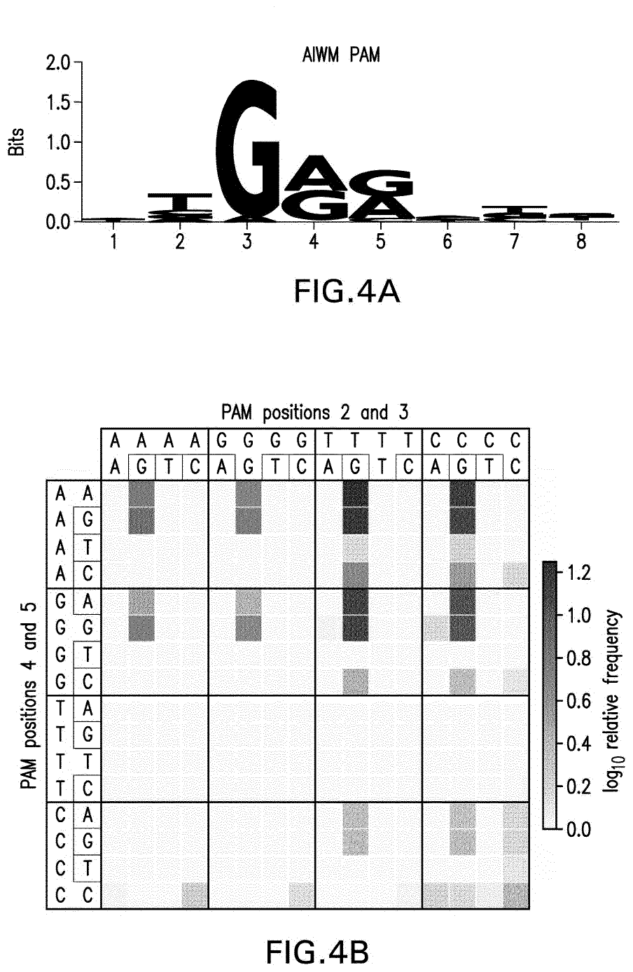

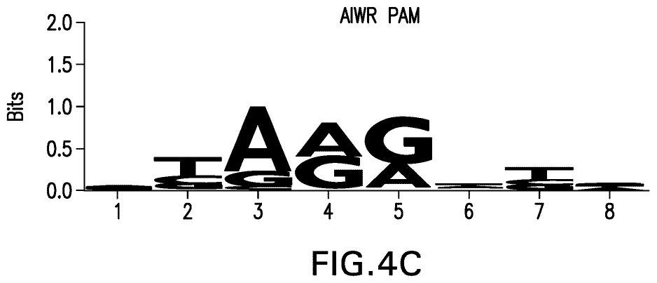

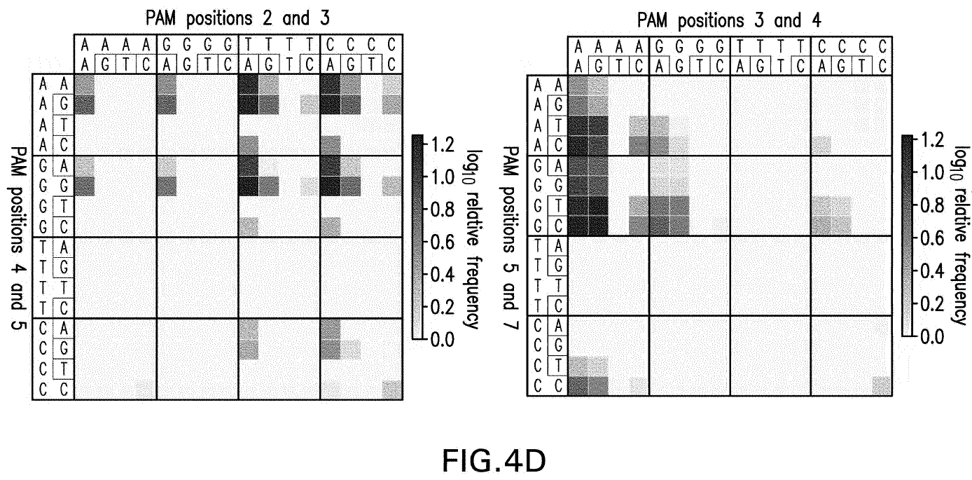

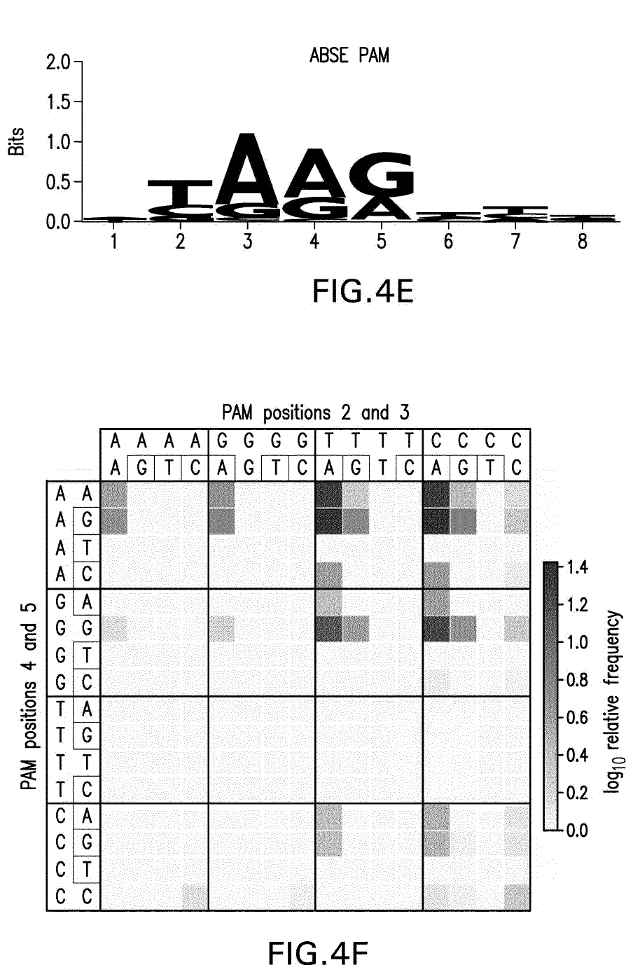

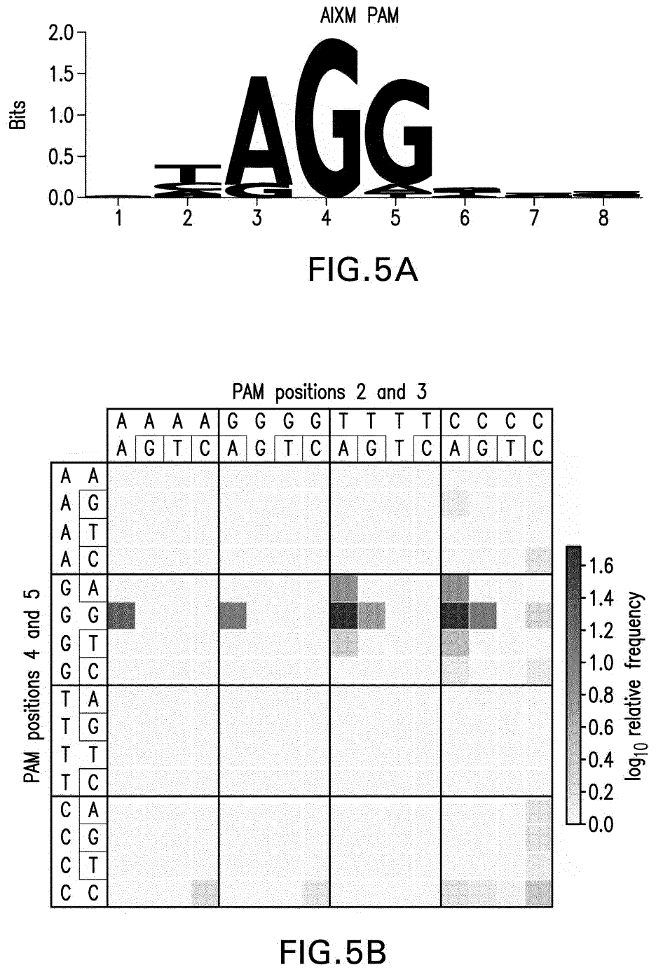

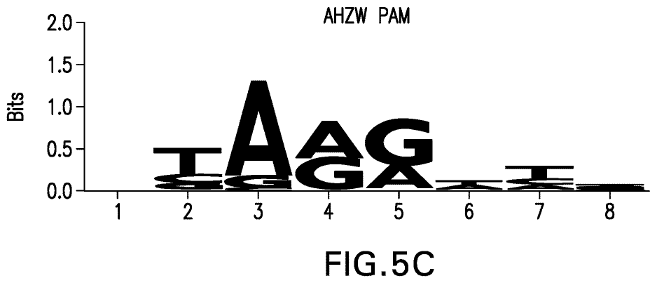

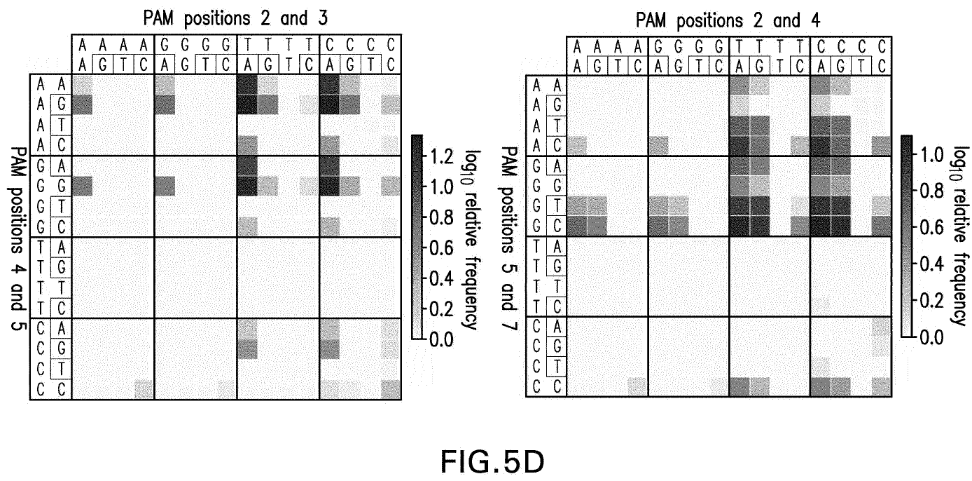

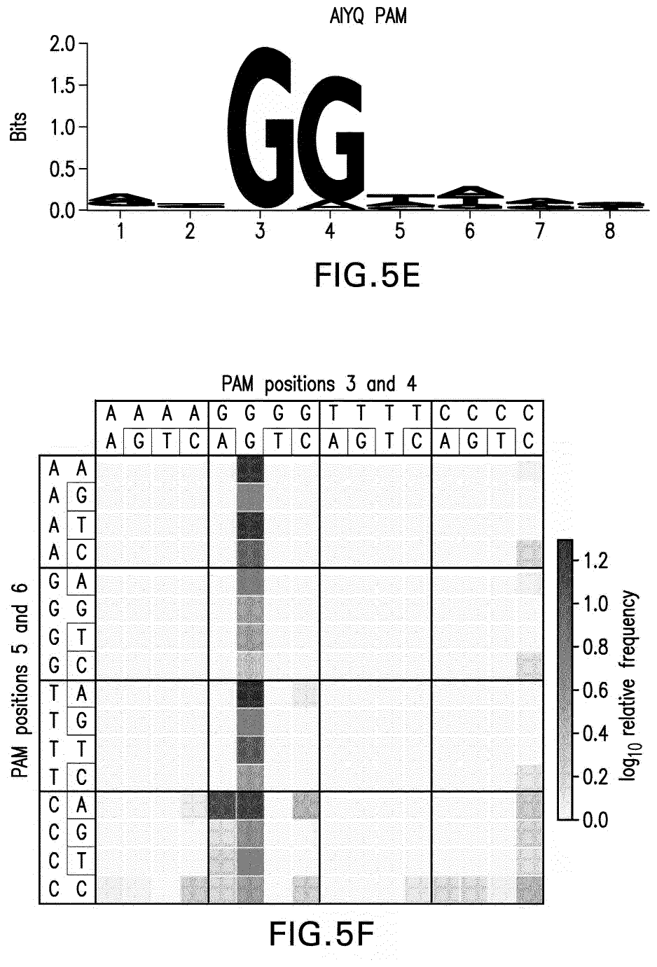

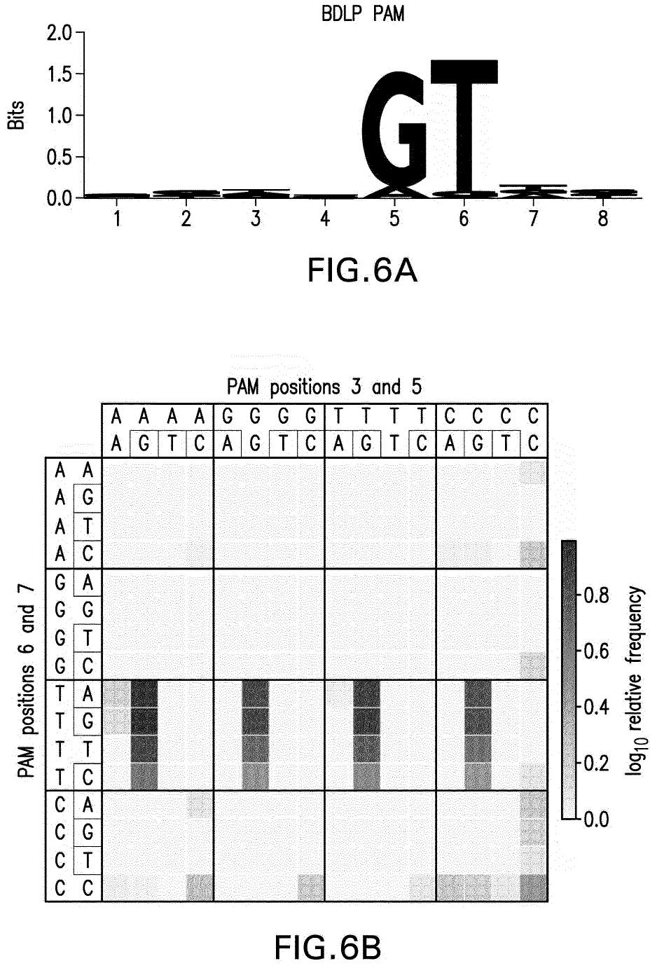

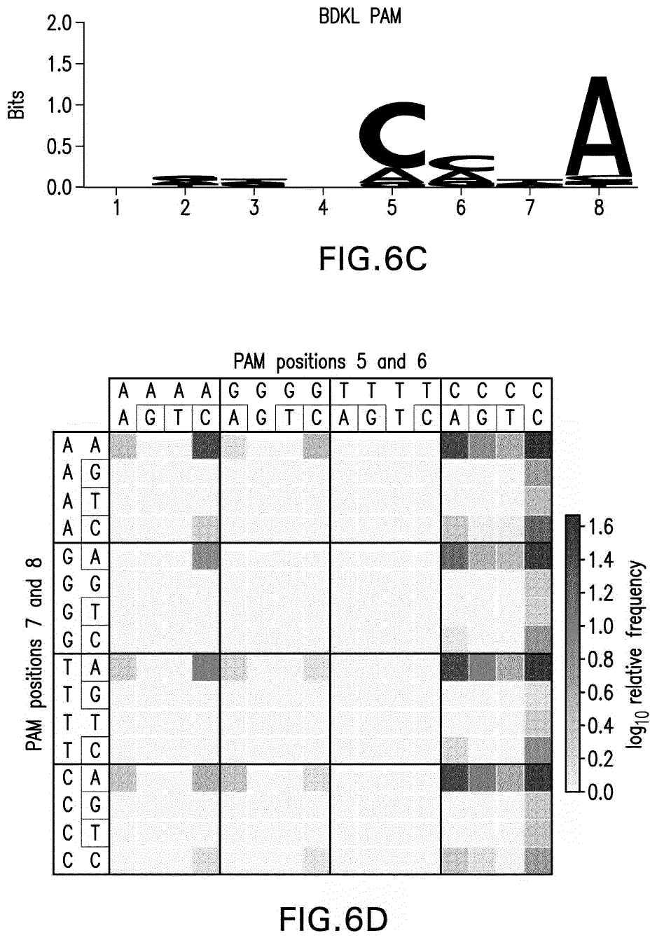

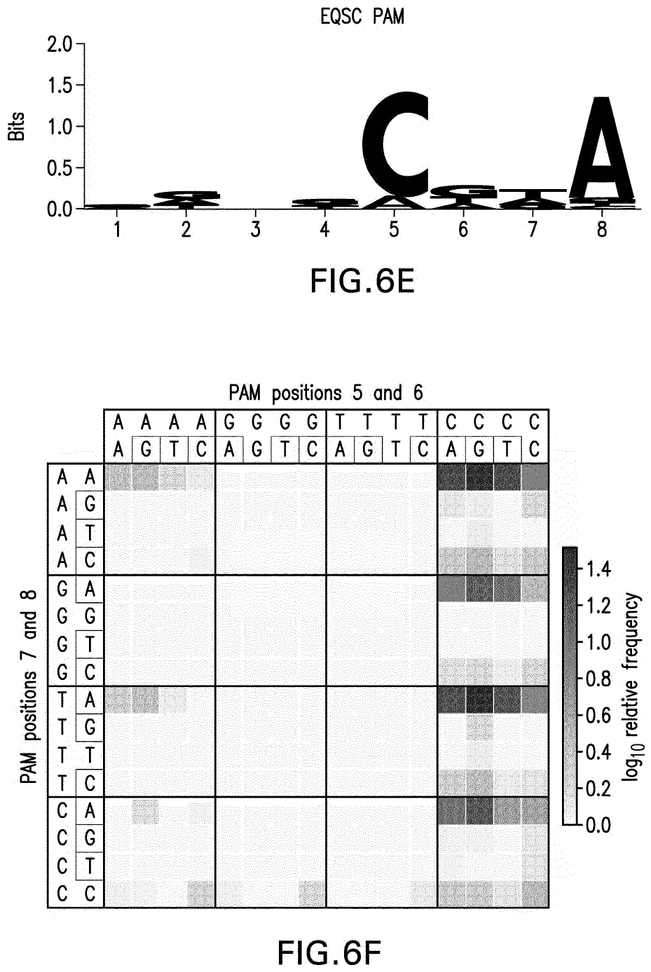

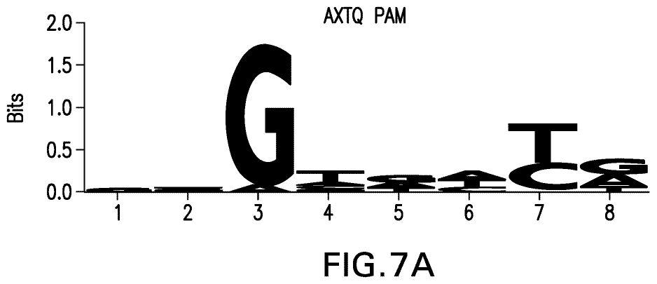

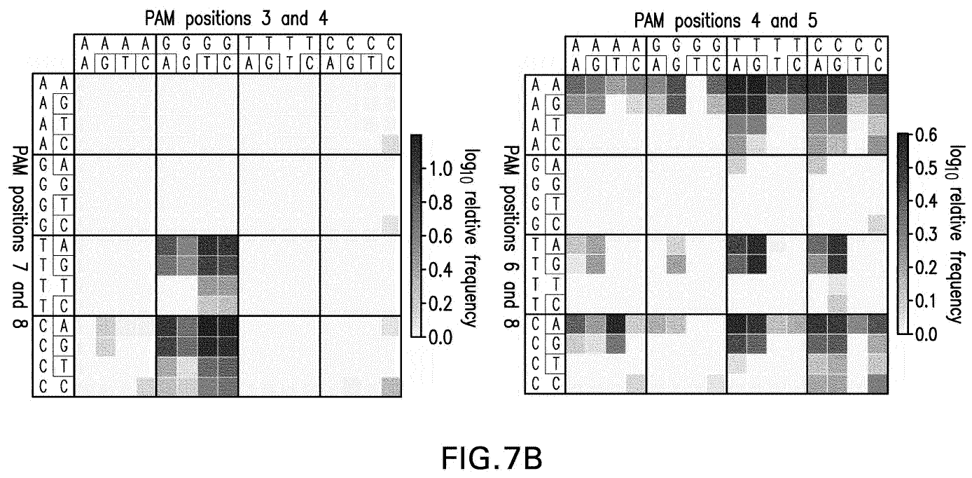

Having determined the sgRNA requirements for the selected Type II Cas proteins identified in Example 1, it was possible to proceed with the in vitro determination of the PAM sites recognized by each nuclease. To this aim the in vitro PAM assay described in Section 7.2.1 was exploited. Briefly, the assay uses in vitro translated Type II Cas proteins coupled with an in vitro synthesized sgRNA to generate a functional ribonucleoprotein complex to cleave a plasmid library characterized by a defined target sequence followed by a randomized 8 nt stretch corresponding to the putative PAMs. Cleaved PAMs could then be recovered after library preparation by next generation sequencing. For most of the PAM assays, in vitro transcribed trimmed versions of the sgRNAs targeting the reporter plasmid were used. Table 12 below contains the PAM preferences as determined based on the assay outcome. The PAM logos and the PAM heatmaps reporting the nucleotide preferences for specific positions along the PAMs are reported in FIGS. 4A-7B.

TABLE 12
PAM preferences as determined by in vitro assay

| Type II Cas protein | in vitro-determined PAM | Most preferred PAMs |
|---|---|---|
| AIWM Type II Cas | NNGRR | NYGRR |
| AIWR Type II Cas | NYRRR, NNARR | NYRRRNY, NYARGNY, NYARRNY |
| ABSE Type II Cas | NYRRR, NNARR | NYAAR, NYARR |
| AIXM Type II Cas | NNRGR | NYRGR, NYAGG, NYRGG, NNAGG |
| AHZW Type II Cas | NYRRR, NNRRR | NYARR, NYAAR, NYARRNY, NYAARNY |
| AIYQ Type II Cas | NNGG | NNGGWW, NNGGAW |
| BDLP Type II Cas | NNNNGT | |
| BDKL Type II Cas | NNNNCNNA, NNNNCC | NNNNCMNA, NNNNCCNA |
| EQSC Type II Cas | NNNNCNNA | NNNNCKNA |
| AXTQ Type II Cas | NNGNNNYN, NNGNNNYR | NNGYNNYR, NNGYNNCR, NNGNVHYR, |
| | | NNGYVHYR, NNGYVHCR, NNGHNHYR |

N = A, T, C, or G; H = A, C, or T; V = A, C, or G; R = G or A; Y = C or T; W = A or T; K = G or T

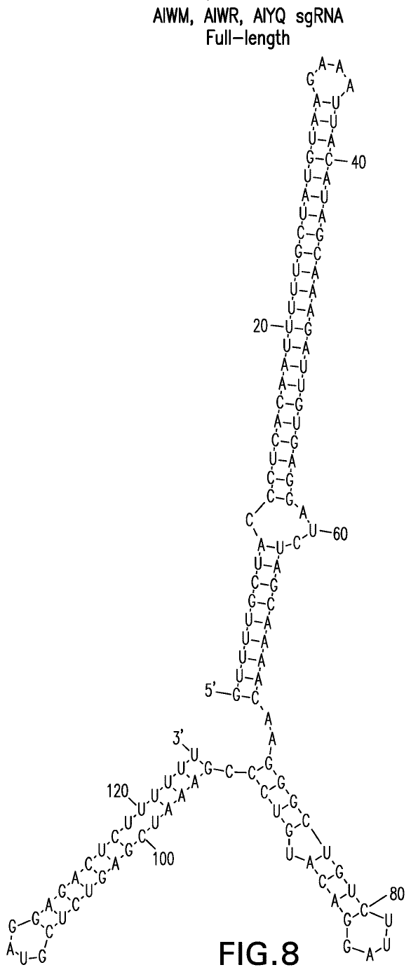

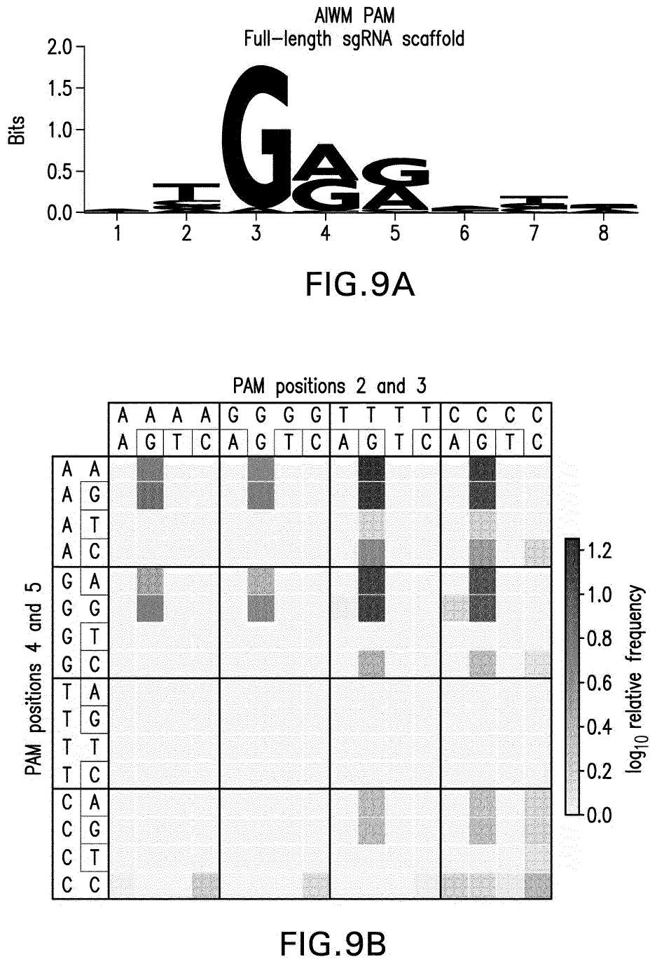

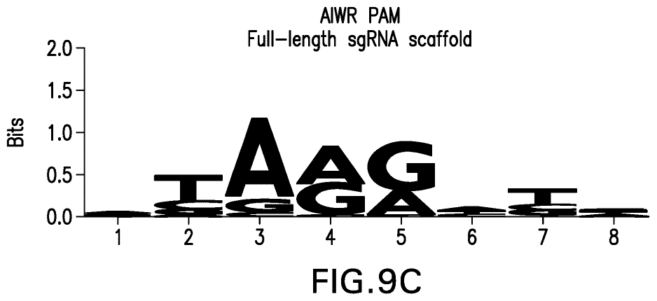

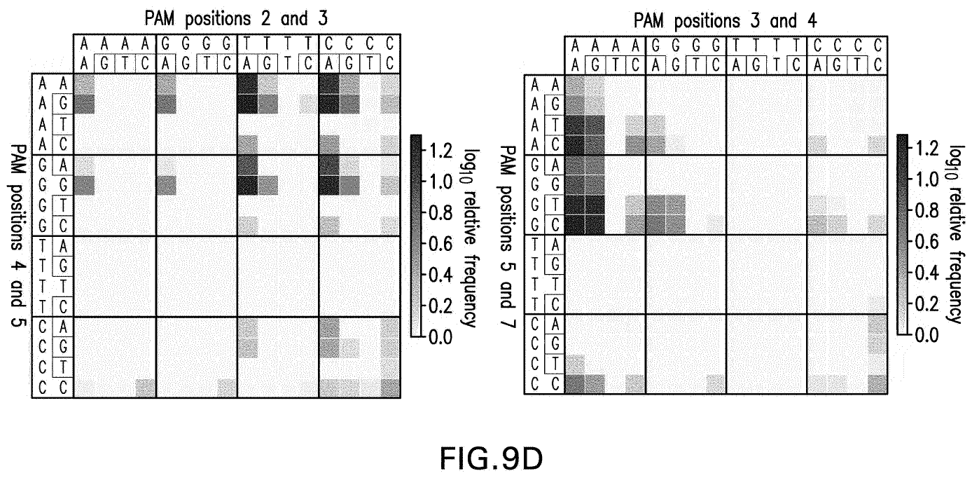

To further confirm the robustness of the generated data, the PAM preferences of AIWM and AIXM Type II Cas proteins were determined using the same PAM assay but exploiting in vitro transcribed full-length sgRNAs (represented in FIG. 8). As shown in FIG. 9, the PAM preferences determined in these conditions matched those previously established using the trimmed version of the same guide RNA.

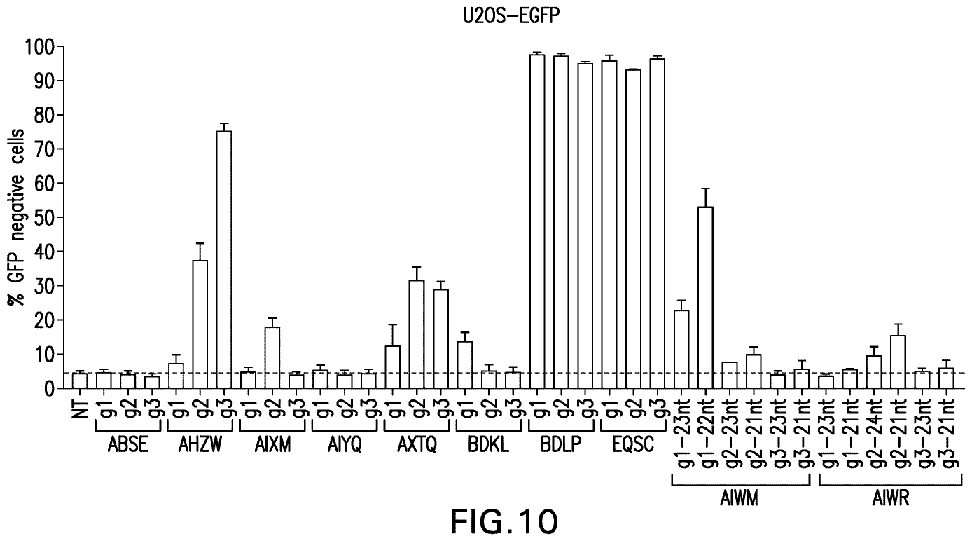

After the discovery of the PAM sequences and the sgRNAs of the selected Type II Cas proteins and after obtaining preliminary information on the ability of these nucleases to cut a desired target in vitro (plasmid target used during the PAM assay), their ability to cleave selected targets in mammalian cells was investigated. An EGFP reporter system was used as it allowed an easier readout on the editing activity, based on the loss of fluorescence of treated cells quantitatively measured by cytofluorimetry. sgRNAs targeting the EGFP coding sequence (three for each evaluated Type II Cas protein) were thus designed for all the Type II Cas proteins and evaluated in U2OS cells stably expressing a single copy of an EGFP reporter by transient electroporation. For AIWM and AIWR Type II Cas proteins, different spacer lengths were also evaluated (range: 21 to 24 matching nucleotides) to evaluate the influence on protein activity. As reported in FIG. 10, it was surprisingly found that some of the evaluated guides in combination with their respective Type II Cas protein were able to significantly downregulate EGFP expression in target cells. In particular, BDLP and EQSC Type II Cas proteins showed very high (>90% EGFP KO) with all the evaluated guides; AIWM, AHZW and AXTQ Type II Cas proteins showed appreciable knock-out activity (>20% EGFP KO) with at least one of the evaluated sgRNAs; AIXM, BDKL and AIWR Type II Cas proteins showed less activity than the aforementioned Type II Cas proteins but still showed some activity (>10% EGFP KO) with at least one of the evaluated guide RNAs. The remaining Type II Cas proteins (ABSE and AIYQ) did not show editing levels above the background of the assay against the currently evaluated targets in the EGFP coding sequence. These data clearly demonstrate that some of the selected Type II Cas proteins were able to very efficiently modify genetic targets in mammalian cells and can thus be exploited to edit the mammalian genome.

### 7.3. Example 3: Allele Specific RHO Editing with EQSC, AHZW, and BDLP Type II Cas Proteins

This Example describes studies performed to evaluate allele-specific editing at the RHO rs7984 locus with EQSC, AHZW, and BDLP Type II Cas proteins.

### 7.3.1. Materials and Methods

### 7.3.1.1. Plasmids

EF1alpha-driven expression plasmids were used to express EQSC, AHZW, and BDLP Type II Cas proteins in mammalian cells. Briefly, the human codon-optimized coding sequences of the different Type II Cas were cloned into the aforementioned expression plasmid. The sgRNA scaffold of each Type II Cas (trimmed scaffold reported in Table 7A or Table 7B, with added 3′ uracils) was cloned into an expression plasmid containing a human U6 promoter to drive guide RNA expression in mammalian cells. Each Type II Cas coding sequence, modified by the addition of an SV5 tag at the N-terminus and two bipartite nuclear localization signals (1 at the N-terminus and 1 at the C-terminus) and human codon-optimized, as well as the sgRNA expression cassettes (U6 promoter+sgRNA scaffolds), were obtained as synthetic constructs from Twist Bioscience. Spacer sequences were cloned into the sgRNA expression plasmids as annealed DNA oligonucleotides using a double Bsal site present in the plasmid. The list of spacer sequences and relative cloning oligonucleotides used in the present example is reported in Table 13.

TABLE 13
Sequences of the oligonucleotides used for cloning sgRNA spacers

| | |
|---|---|
| Spacer sequences | Oligo used to clone the spacer in the sgRNA plasmid (*) |

| | | SEQ ID | | SEQ ID | | SEQ ID |
|---|---|---|---|---|---|---|
| Name | Protospacer | NO: | Oligo 1 (5′→3′) | NO: | Oligo 2 (5′→3′) | NO: |
| gRNA-EQSC- | TTGTGGCTGACCC | 337 | caccgTTGTGGCTGA | 345 | agacGAGCAGCCAC | 353 |
| RHOrs7984A- | GTGGCTGCTC | | CCCGTGGCTGCTC | | GGGTCAGCCACAAc | |
| g1 | | | | | | |
| gRNA-EQSC- | CTTGTGGCTGACC | 338 | caccgCTTGTGGCTG | 346 | agacAGCAGCCACG | 354 |
| RHOrs7984A- | CGTGGCTGCT | | ACCCGTGGCTGCT | | GGTCAGCCACAAGc | |
| g2 | | | | | | |
| gRNA-EQSC- | GGCCCTTGTGGCT | 339 | caccGGCCCTTGTGG | 347 | agacGCCACGGGTC | 355 |
| RHOrs7984A- | GACCCGTGGC | | CTGACCCGTGGC | | AGCCACAAGGGCC | |
| g3 | | | | | | |
| gRNA-EQSC- | CTTGGGTGGGAGC | 340 | caccgCTTGGGTGGG | 348 | agacACCCGTGGCTG | 356 |
| RHOrs7984A- | AGCCACGGGT | | AGCAGCCACGGGT | | CTCCCACCCAAGc | |
| g4 | | | | | | |
| gRNA-EQSC- | GAGCAGCCACGGG | 341 | caccGAGCAGCCACG | 349 | agacTTGTGGCTGAC | 357 |
| RHOrs7984A- | TCAGCCACAA | | GGTCAGCCACAA | | CCGTGGCTGCTC | |
| g5 | | | | | | |
| gRNA-BDLP- | CATGGCTGTGGCC | 342 | caccgCATGGCTGTG | 350 | agacCAGCCACAAGG | 358 |
| RHOrs7984A- | CTTGTGGCTG | | GCCCTTGTGGCTG | | GCCACAGCCATGc | |
| g1 | | | | | | |
| gRNA-AHZW- | GTGGGAGCAGCCA | 343 | caccGTGGGAGCAGC | 351 | gaacGGCTGACCCGT | 359 |
| rs7984A-g1 | CGGGTCAGCC | | CACGGGTCAGCC | | GGCTGCTCCCAC | |
| gRNA-AHZW- | GGCTGACCCGTGG | 344 | caccGGCTGACCCGT | 352 | gaacGTGGGAGCAG | 360 |
| rs7984A-g2 | CTGCTCCCAC | | GGCTGCTCCCAC | | CCACGGGTCAGCC | |

(*) The cloning overhang is reported in lowercase. Nucleotides highlighted in bold represent 5′-G appended to favor transcription from canonical U6 Pol III promoter

### 7.3.1.2. Cell Lines

HEK293T cells (obtained from ATCC) were cultured in DMEM (Life Technologies) supplemented with 10% FBS (Life Technologies), 2 mM L-Glutamine (Life Technologies) and penicillin/streptomycin (Life Technologies). All cells were incubated at 37° C. and 5% CO₂ in a humidified atmosphere. All cells tested mycoplasma negative (PlasmoTest™, Invivogen).

### 7.3.1.3. Cell Line Transfections

To perform editing studies on target RHO locus 100,000 HEK293T cells were seeded in a 24-well plate 24 hours before transfection. Cells were then transfected with 500 ng of nuclease-expressing plasmid together with 250 ng of sgRNA expression vector targeting the locus of interest using the TransIT®-LT1 reagent (Mirus Bio) according to the manufacturer's protocol. Cell pellets were collected 3 day from transfection for analysis.

### 7.3.1.4. Evaluation of Gene Editing

Three days after transfection cells were collected and DNA was extracted using the QuickExtract™ DNA Extraction Solution (Lucigen) according to the manufacturer's instructions. To amplify the target loci, PCR reactions were performed using the HOT FIREPol® polymerase (Solis BioDyne) and the oligonucleotides listed in Table 14. The amplified products were purified, sent for Sanger sequencing (EasyRun service, Microsynth) and analyzed with the TIDE web tool (shinyapps.datacurators.nl/tide/) to quantify indels. The primers used for Sanger sequencing reactions on amplicons are reported in Table 15, associated with their respective target locus.

TABLE 14
Oligonucleotides used to amplify target regions to
perform TIDE analysis

| Locus | For (5′ → 3′) | Rev (5′ → 3′) |
|---|---|---|
| rs7984 | GGGAACGAACAGGTAAGGG | AGCGTGAGGAAGTTGATGG |
| endogenous | G (SEQ ID NO: 361) | G (SEQ ID NO: 362) |

TABLE 15
Oligonucleotides used for Sanger sequencing to
perform TIDE analysis

| | |
|---|---|
| rs7984 endogenous | AGCGTGAGGAAGTTGATGGG |
| | (SEQ ID NO: 362) |

### 7.3.2. Results

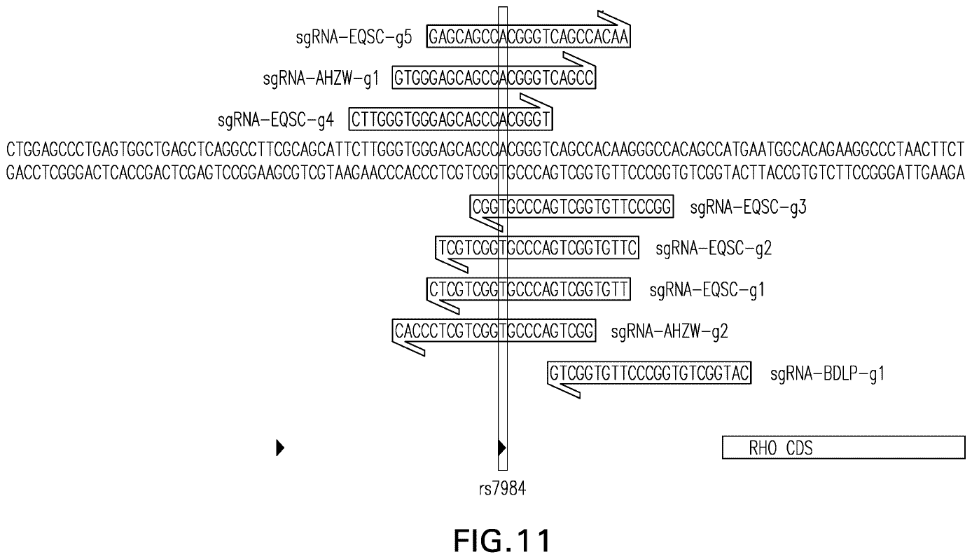

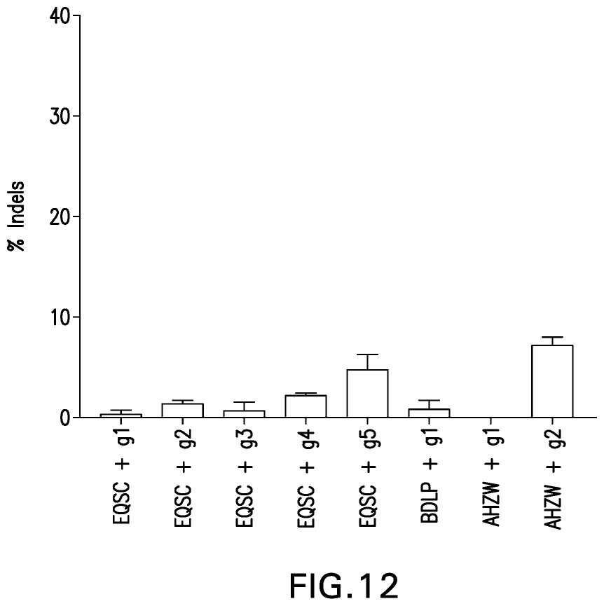

A set of sgRNAs associated with PAMs strongly recognized by EQSC, AHZW and BDLP Type II Cas spanning the rs7984 SNP were designed (FIG. 11). The editing activity of the selected guides in combination with the respective nucleases was evaluated by transient transfection of HEK293T cells. These cells are homozygous for the rs7984A allele of the SNP and sgRNAs targeting the rs7984A allele were used. As shown in FIG. 12, besides BDLP Type II Cas which did not show appreciable indel formation in this study, for both EQSC and AHZW Type II Cas at least one sgRNA was identified producing editing levels which were above the background of the readout assay.

### 7.4. Example 4: Gene Editing with BDLP and EQSC Type II Cas Proteins

To extensively evaluate the cleavage activity BDLP and EQSC Type II Cas, a panel of endogenous loci (B2M, TRAC, PD-1) which are commonly targeted to generate allogeneic CAR-T cells (Chimeric Antigen Receptor T cells) were selected for editing studies. For each target locus multiple sgRNAs were designed and evaluated in parallel by transient plasmid transfection in HEK293T cells.

Materials and methods were similar to those used in Example 3. Table 16 shows protospacer and oligo sequences used for cloning sgRNA spacers. Table 17 shows oligos used for TIDE analysis.

TABLE 16
Sequences of the oligonucleotides used for cloning sgRNA spacers

| Spacer sequences | | | Oligo used to clone the spacer in the sgRNA plasmid (*) | | | |
|---|---|---|---|---|---|---|
| | | SEQ ID | | SEQ ID | | SEQ ID |
| Name | Protospacer | NO: | Oligo 1 (5′→3′) | NO: | Oligo 2 (5′→3′) | NO |
| gRNA-EQSC- | CAGAAAGAGAGA | 363 | caccgCAGAAAGAGAG | 391 | agacGCTCGCGCTAC | 419 |
| B2M-g1 | GTAGCGCGAGC | | AGTAGCGCGAGC | | TCTCTCTTTCTGC | |
| gRNA-EQSC- | CACGTCATCCAG | 364 | caccgCACGTCATCCA | 392 | agacTCCATTCTCTGC | 420 |
| B2M-g2 | CAGAGAATGGA | | GCAGAGAATGGA | | TGGATGACGTGC | |
| gRNA-EQSC- | CATTCTTCAGTAA | 365 | caccgCATTCTTCAGTA | 393 | agacTGAAGTTGACTT | 421 |
| B2M-g3 | GTCAACTTCA | | AGTCAACTTCA | | ACTGAAGAATGc | |
| gRNA-BDLP- | AGCATTCGGGCC | 366 | caccgAGCATTCGGGC | 394 | agacCGAGACATCTC | 422 |
| B2M-g1 | GAGATGTCTCG | | CGAGATGTCTCG | | GGCCCGAATGCTc | |
| gRNA-BDLP- | GAGATGTCTCGC | 367 | caccGAGATGTCTCGC | 395 | agacAAGGCCACGGA | 423 |
| B2M-g2 | TCCGTGGCCTT | | TCCGTGGCCTT | | GCGAGACATCTC | |
| gRNA-BDLP- | GGATAGCCTCCA | 368 | caccGGATAGCCTCCA | 396 | agacTCTTTCTGGCCT | 424 |
| B2M-g3 | GGCCAGAAAGA | | GGCCAGAAAGA | | GGAGGCTATCC | |
| gRNA-BDLP- | TTGACTTTCCATT | 369 | caccgTTGACTTTCCAT | 397 | agacTCCAGCAGAGA | 425 |
| B2M-g4 | CTCTGCTGGA | | TCTCTGCTGGA | | ATGGAAAGTCAAc | |
| gRNA-BDLP- | GGAAAGTCAAAT | 370 | caccGGAAAGTCAAAT | 398 | agacCAATTCAGGAA | 426 |
| B2M-g5 | TTCCTGAATTG | | TTCCTGAATTG | | ATTTGACTTTCC | |
| gRNA-EQSC- | TTTGTCTGTGATA | 371 | caccgTTTGTCTGTGAT | 399 | agacCTGATGTGTATA | 427 |
| TRAC-g1 | TACACATCAG | | ATACACATCAG | | TCACAGACAAAC | |
| gRNA-EQSC- | GTAAGGATTCTG | 372 | caccGTAAGGATTCTG | 400 | agacTGATATACACAT | 428 |
| TRAC-g2 | ATGTGTATATCA | | ATGTGTATATCA | | CAGAATCCTTAC | |
| gRNA-EQSC- | GGCCACAGCACT | 373 | caccGGCCACAGCACT | 401 | agacTCAAGAGCAAC | 429 |
| TRAC-g3 | GTTGCTCTTGA | | GTTGCTCTTGA | | AGTGCTGTGGCC | |
| gRNA-EQSC- | GAGCAACAGTGC | 374 | caccGAGCAACAGTGC | 402 | agacTCCAGGCCACA | 430 |
| TRAC-g4 | TGTGGCCTGGA | | TGTGGCCTGGA | | GCACTGTTGCTC | |
| gRNA-EQSC- | GGAGCAACAAAT | 375 | caccGGAGCAACAAAT | 403 | agacTGCAAAGTCAG | 431 |
| TRAC-g5 | CTGACTTTGCA | | CTGACTTTGCA | | ATTTGTTGCTCC | |
| gRNA-BDLP- | AATGTGTCACAA | 376 | caccgAATGTGTCACA | 404 | agacGAATCCTTACTT | 432 |
| TRAC-g1 | AGTAAGGATTC | | AAGTAAGGATTC | | TGTGACACATTC | |
| gRNA-BDLP- | TCTGATGTGTATA | 377 | caccgTCTGATGTGTAT | 405 | agacTTGTCTGTGATA | 433 |
| TRAC-g2 | TCACAGACAA | | ATCACAGACAA | | TACACATCAGAC | |
| gRNA-BDLP- | GCCTGGAGCAAC | 378 | caccGCCTGGAGCAAC | 406 | agacAAAGTCAGATTT | 434 |
| TRAC-g3 | AAATCTGACTTT | | AAATCTGACTTT | | GTTGCTCCAGGC | |
| gRNA-BDLP- | GGCGTTTGCACA | 379 | caccGGCGTTTGCACA | 407 | agacCTGACTTTGCAT | 435 |
| TRAC-g4 | TGCAAAGTCAG | | TGCAAAGTCAG | | GTGCAAACGCC | |
| gRNA-BDLP- | GAAGAAGGTGTC | 380 | caccGAAGAAGGTGTC | 408 | agacTTATTCCAGAAG | 436 |
| TRAC-g5 | TTCTGGAATAA | | TTCTGGAATAA | | ACACCTTCTTC | |
| gRNA-BDLP- | GCTGCCCTTACC | 381 | caccGCTGCCCTTACC | 409 | agacTTCCCCAGCCC | 437 |
| TRAC-g6 | TGGGCTGGGGAA | | TGGGCTGGGGAA | | AGGTAAGGGCAGC | |
| gRNA-BDLP- | TGGGCTGGCGGC | 382 | caccgTGGGCTGGCGG | 410 | agacGAACCATCCTG | 438 |
| PD1-g1 | CAGGATGGTTC | | CCAGGATGGTTC | | GCCGCCAGCCCAC | |
| gRNA-BDLP- | CCGCCCAGACGA | 383 | caccgCCGCCCAGACG | 411 | agacGCCCTGGCCAG | 439 |
| PD1-g2 | CTGGCCAGGGC | | ACTGGCCAGGGC | | TCGTCTGGGCGGC | |
| gRNA-BDLP- | CAGGCGCCCTGG | 384 | caccgCAGGCGCCCTG | 412 | agacCAGACGACTGG | 440 |
| PD1-g3 | CCAGTCGTCTG | | GCCAGTCGTCTG | | CCAGGGCGCCTGc | |
| gRNA-BDLP- | AGAACCATCCTG | \|385 | caccgAGAACCATCCT | 413 | agacGGGCTGGCGG | 441 |
| PD1-g4 | GCCGCCAGCCC | | GGCCGCCAGCCC | | CCAGGATGGTTCTC | |
| gRNA-BDLP- | CTAAGAACCATC | 386 | caccgCTAAGAACCAT | 414 | agacCTGGCGGCCAG | 442 |
| PD1-g5 | CTGGCCGCCAG | | CCTGGCCGCCAG | | GATGGTTCTTAGC | |
| gRNA-BDLP- | GCTGGCGGCCA | 387 | caccGCTGGCGGCCA | 415 | agacCTAAGAACCAT | 443 |
| PD1-g6 | GGATGGTTCTTA | | GGATGGTTCTTAG | | CCTGGCCGCCAGC | |
| | G | | | | | |
| gRNA-EQSC- | ACAACTGGGCTG | 388 | caccgACAACTGGGCT | 416 | agacATCCTGGCCGC | 444 |
| PD1-g1 | GCGGCCAGGAT | | GGCGGCCAGGAT | | CAGCCCAGTTGTc | |
| gRNA-EQSC- | GTGGGGCTGCTC | 389 | caccGTGGGGCTGCTC | 417 | agacCTGCATGCCTG | 445 |
| PD1-g2 | CAGGCATGCAG | | CAGGCATGCAG | | GAGCAGCCCCAC | |
| gRNA-EQSC- | GGCCGCCAGCC | 390 | caccGGCCGCCAGCC | 418 | agacGTGCTACAACT | 446 |
| PD1-g3 | CAGTTGTAGCAC | | CAGTTGTAGCAC | | GGGCTGGCGGCC | |

(*) The cloning overhang is reported in lowercase. Nucleotides highlighted in bold represent 5′-G appended to favor transcription from canonical U6 Pol III promoter

TABLE 17
Oligonucleotides used to amplify target regions to
perform TIDE analysis

| Locus | For (5′ → 3′) | Rev (5′ → 3′) |
|---|---|---|
| TRAC | CATCACGAGCAGCTGGTTTC | TGGCAATGGATAAGGCCGAG |
| | (SEQ ID NO: 447) | (SEQ ID NO: 451) |
| B2M | CTCTAACCTGGCACTGCGTC | CGAAGTCCACAGCTCTCCAG |
| ex1 | (SEQ ID NO: 448) | (SEQ ID NO: 452) |
| B2M | TGGCCAGAGTGGAAATGGAA | TGTATTTGTGCAAGTGCTGCT |
| ex2 | (SEQ ID NO: 449) | (SEQ ID NO: 453) |
| PD-1 | CACTGCCTCTGTCACTCTCG | GACTTGGGCCAGGGGAGGAG |
| | (SEQ ID NO: 450) | (SEQ ID ID NO: 454) |

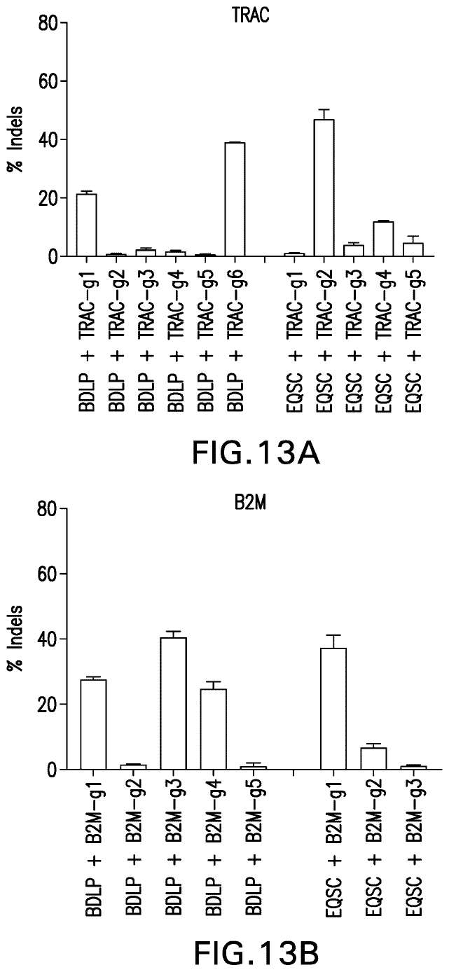

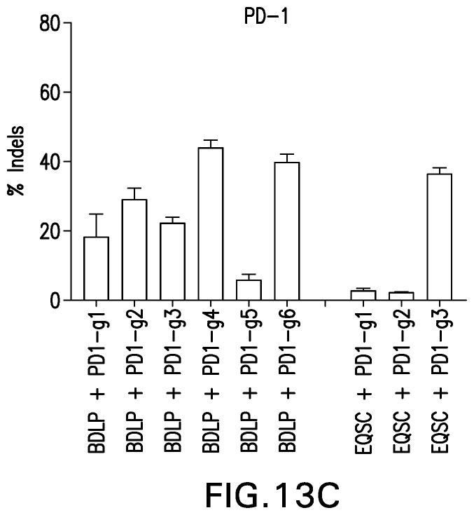

As shown in FIGS. 13A-13C, for each of the evaluated loci, both nucleases showed significant editing activity (near or above 40% indel formation) with at least one of the selected guide RNAs, demonstrating that these novel Type II Cas proteins have the ability to effectively modify genomic targets of interest.

### 8. SPECIFIC EMBODIMENTS

The present disclosure is exemplified by the specific embodiments below.

1. A Type II Cas protein comprising an amino acid sequence having at least 50% sequence identity to:
(a) the amino acid sequence of a RuvC-I domain of a reference protein sequence;
(b) the amino acid sequence of a RuvC-II domain of a reference protein sequence;
(c) the amino acid sequence of a RuvC-III domain of a reference protein sequence;
(d) the amino acid sequence of a BH domain of a reference protein sequence;
(e) the amino acid sequence of a REC domain of a reference protein sequence;
(f) the amino acid sequence of a HNH domain of a reference protein sequence;
(g) the amino acid sequence of a WED domain of a reference protein sequence;
(h) the amino acid sequence of a PID domain of a reference protein sequence; or
(i) the amino acid sequence of the full length of a reference protein sequence;
wherein the reference protein sequence is SEQ ID NO:1, SEQ ID NO:2, SEQ ID NO:7, SEQ ID NO:8, SEQ ID NO:13, SEQ ID NO:14, SEQ ID NO:19, SEQ ID NO:20, SEQ ID NO:25, SEQ ID NO:26, SEQ ID NO:31, SEQ ID NO:32, SEQ ID NO:37, SEQ ID NO:38, SEQ ID NO:43, SEQ ID NO:44, SEQ ID NO:49, SEQ ID NO:50, SEQ ID NO:55, or SEQ ID NO:56.

2. The Type II Cas protein of embodiment 1, wherein the amino acid sequence of the Type II Cas protein comprises an amino acid sequence that is at least 50% identical to the amino acid sequence of the RuvC-I domain of the reference protein sequence.

3. The Type II Cas protein of embodiment 1, wherein the amino acid sequence of the Type II Cas protein comprises an amino acid sequence that is at least 55% identical to the amino acid sequence of the RuvC-I domain of the reference protein sequence.

4. The Type II Cas protein of embodiment 1, wherein the amino acid sequence of the Type II Cas protein comprises an amino acid sequence that is at least 60% identical to the amino acid sequence of the RuvC-I domain of the reference protein sequence.

5. The Type II Cas protein of embodiment 1, wherein the amino acid sequence of the Type II Cas protein comprises an amino acid sequence that is at least 65% identical to the amino acid sequence of the RuvC-I domain of the reference protein sequence.

6. The Type II Cas protein of embodiment 1, wherein the amino acid sequence of the Type II Cas protein comprises an amino acid sequence that is at least 70% identical to the amino acid sequence of the RuvC-I domain of the reference protein sequence.

7. The Type II Cas protein of embodiment 1, wherein the amino acid sequence of the Type II Cas protein comprises an amino acid sequence that is at least 75% identical to the amino acid sequence of the RuvC-I domain of the reference protein sequence.

8. The Type II Cas protein of embodiment 1, wherein the amino acid sequence of the Type II Cas protein comprises an amino acid sequence that is at least 80% identical to the amino acid sequence of the RuvC-I domain of the reference protein sequence.

9. The Type II Cas protein of embodiment 1, wherein the amino acid sequence of the Type II Cas protein comprises an amino acid sequence that is at least 85% identical to the amino acid sequence of the RuvC-I domain of the reference protein sequence.

10. The Type II Cas protein of embodiment 1, wherein the amino acid sequence of the Type II Cas protein comprises an amino acid sequence that is at least 90% identical to the amino acid sequence of the RuvC-I domain of the reference protein sequence.

11. The Type II Cas protein of embodiment 1, wherein the amino acid sequence of the Type II Cas protein comprises an amino acid sequence that is at least 95% identical to the amino acid sequence of the RuvC-I domain of the reference protein sequence.

12. The Type II Cas protein of embodiment 1, wherein the amino acid sequence of the Type II Cas protein comprises an amino acid sequence that is at least 96% identical to the amino acid sequence of the RuvC-I domain of the reference protein sequence.

13. The Type II Cas protein of embodiment 1, wherein the amino acid sequence of the Type II Cas protein comprises an amino acid sequence that is at least 97% identical to the amino acid sequence of the RuvC-I domain of the reference protein sequence.

14. The Type II Cas protein of embodiment 1, wherein the amino acid sequence of the Type II Cas protein comprises an amino acid sequence that is at least 98% identical to the amino acid sequence of the RuvC-I domain of the reference protein sequence.

15. The Type II Cas protein of embodiment 1, wherein the amino acid sequence of the Type II Cas protein comprises an amino acid sequence that is at least 99% identical to the amino acid sequence of the RuvC-I domain of the reference protein sequence.

16. The Type II Cas protein of embodiment 1, wherein the amino acid sequence of the Type II Cas protein comprises an amino acid sequence that is identical to the amino acid sequence of the RuvC-I domain of the reference protein sequence.

17. The Type II Cas protein of any one of embodiments 1 to 16, wherein the amino acid sequence of the Type II Cas protein comprises an amino acid sequence that is at least 50% identical to the amino acid sequence of the RuvC-II domain of the reference protein sequence.

18. The Type II Cas protein of any one of embodiments 1 to 16, wherein the amino acid sequence of the Type II Cas protein comprises an amino acid sequence that is at least 55% identical to the amino acid sequence of the RuvC-II domain of the reference protein sequence.

19. The Type II Cas protein of any one of embodiments 1 to 16, wherein the amino acid sequence of the Type II Cas protein comprises an amino acid sequence that is at least 60% identical to the amino acid sequence of the RuvC-II domain of the reference protein sequence.

20. The Type II Cas protein of any one of embodiments 1 to 16, wherein the amino acid sequence of the Type II Cas protein comprises an amino acid sequence that is at least 65% identical to the amino acid sequence of the RuvC-II domain of the reference protein sequence.

21. The Type II Cas protein of any one of embodiments 1 to 16, wherein the amino acid sequence of the Type II Cas protein comprises an amino acid sequence that is at least 70% identical to the amino acid sequence of the RuvC-II domain of the reference protein sequence.

22. The Type II Cas protein of any one of embodiments 1 to 16, wherein the amino acid sequence of the Type II Cas protein comprises an amino acid sequence that is at least 75% identical to the amino acid sequence of the RuvC-II domain of the reference protein sequence.

23. The Type II Cas protein of any one of embodiments 1 to 16, wherein the amino acid sequence of the Type II Cas protein comprises an amino acid sequence that is at least 80% identical to the amino acid sequence of the RuvC-II domain of the reference protein sequence.

24. The Type II Cas protein of any one of embodiments 1 to 16, wherein the amino acid sequence of the Type II Cas protein comprises an amino acid sequence that is at least 85% identical to the amino acid sequence of the RuvC-II domain of the reference protein sequence.

25. The Type II Cas protein of any one of embodiments 1 to 16, wherein the amino acid sequence of the Type II Cas protein comprises an amino acid sequence that is at least 90% identical to the amino acid sequence of the RuvC-II domain of the reference protein sequence.

26. The Type II Cas protein of any one of embodiments 1 to 16, wherein the amino acid sequence of the Type II Cas protein comprises an amino acid sequence that is at least 95% identical to the amino acid sequence of the RuvC-II domain of the reference protein sequence.

27. The Type II Cas protein of any one of embodiments 1 to 16, wherein the amino acid sequence of the Type II Cas protein comprises an amino acid sequence that is at least 96% identical to the amino acid sequence of the RuvC-II domain of the reference protein sequence.

28. The Type II Cas protein of any one of embodiments 1 to 16, wherein the amino acid sequence of the Type II Cas protein comprises an amino acid sequence that is at least 97% identical to the amino acid sequence of the RuvC-II domain of the reference protein sequence.

29. The Type II Cas protein of any one of embodiments 1 to 16, wherein the amino acid sequence of the Type II Cas protein comprises an amino acid sequence that is at least 98% identical to the amino acid sequence of the RuvC-II domain of the reference protein sequence.

30. The Type II Cas protein of any one of embodiments 1 to 16, wherein the amino acid sequence of the Type II Cas protein comprises an amino acid sequence that is at least 99% identical to the amino acid sequence of the RuvC-II domain of the reference protein sequence.

31. The Type II Cas protein of any one of embodiments 1 to 16, wherein the amino acid sequence of the Type II Cas protein comprises an amino acid sequence that is identical to the amino acid sequence of the RuvC-II domain of the reference protein sequence.

32. The Type II Cas protein of any one of embodiments 1 to 31, wherein the amino acid sequence of the Type II Cas protein comprises an amino acid sequence that is at least 50% identical to the amino acid sequence of the RuvC-III domain of the reference protein sequence.

33. The Type II Cas protein of any one of embodiments 1 to 31, wherein the amino acid sequence of the Type II Cas protein comprises an amino acid sequence that is at least 55% identical to the amino acid sequence of the RuvC-III domain of the reference protein sequence.

34. The Type II Cas protein of any one of embodiments 1 to 31, wherein the amino acid sequence of the Type II Cas protein comprises an amino acid sequence that is at least 60% identical to the amino acid sequence of the RuvC-III domain of the reference protein sequence.

35. The Type II Cas protein of any one of embodiments 1 to 31, wherein the amino acid sequence of the Type II Cas protein comprises an amino acid sequence that is at least 65% identical to the amino acid sequence of the RuvC-III domain of the reference protein sequence.

36. The Type II Cas protein of any one of embodiments 1 to 31, wherein the amino acid sequence of the Type II Cas protein comprises an amino acid sequence that is at least 70% identical to the amino acid sequence of the RuvC-III domain of the reference protein sequence.

37. The Type II Cas protein of any one of embodiments 1 to 31, wherein the amino acid sequence of the Type II Cas protein comprises an amino acid sequence that is at least 75% identical to the amino acid sequence of the RuvC-III domain of the reference protein sequence.

38. The Type II Cas protein of any one of embodiments 1 to 31, wherein the amino acid sequence of the Type II Cas protein comprises an amino acid sequence that is at least 80% identical to the amino acid sequence of the RuvC-III domain of the reference protein sequence.

39. The Type II Cas protein of any one of embodiments 1 to 31, wherein the amino acid sequence of the Type II Cas protein comprises an amino acid sequence that is at least 85% identical to the amino acid sequence of the RuvC-III domain of the reference protein sequence.

40. The Type II Cas protein of any one of embodiments 1 to 31, wherein the amino acid sequence of the Type II Cas protein comprises an amino acid sequence that is at least 90% identical to the amino acid sequence of the RuvC-III domain of the reference protein sequence.

41. The Type II Cas protein of any one of embodiments 1 to 31, wherein the amino acid sequence of the Type II Cas protein comprises an amino acid sequence that is at least 95% identical to the amino acid sequence of the RuvC-III domain of the reference protein sequence.

42. The Type II Cas protein of any one of embodiments 1 to 31, wherein the amino acid sequence of the Type II Cas protein comprises an amino acid sequence that is at least 96% identical to the amino acid sequence of the RuvC-III domain of the reference protein sequence.

43. The Type II Cas protein of any one of embodiments 1 to 31, wherein the amino acid sequence of the Type II Cas protein comprises an amino acid sequence that is at least 97% identical to the amino acid sequence of the RuvC-III domain of the reference protein sequence.

44. The Type II Cas protein of any one of embodiments 1 to 31, wherein the amino acid sequence of the Type II Cas protein comprises an amino acid sequence that is at least 98% identical to the amino acid sequence of the RuvC-III domain of the reference protein sequence.

45. The Type II Cas protein of any one of embodiments 1 to 31, wherein the amino acid sequence of the Type II Cas protein comprises an amino acid sequence that is at least 99% identical to the amino acid sequence of the RuvC-III domain of the reference protein sequence.

46. The Type II Cas protein of any one of embodiments 1 to 31, wherein the amino acid sequence of the Type II Cas protein comprises an amino acid sequence that is identical to the amino acid sequence of the RuvC-III domain of the reference protein sequence.

47. The Type II Cas protein of any one of embodiments 1 to 46, wherein the amino acid sequence of the Type II Cas protein comprises an amino acid sequence that is at least 50% identical to the amino acid sequence of the BH domain of the reference protein sequence.

48. The Type II Cas protein of any one of embodiments 1 to 46, wherein the amino acid sequence of the Type II Cas protein comprises an amino acid sequence that is at least 55% identical to the amino acid sequence of the BH domain of the reference protein sequence.

49. The Type II Cas protein of any one of embodiments 1 to 46, wherein the amino acid sequence of the Type II Cas protein comprises an amino acid sequence that is at least 60% identical to the amino acid sequence of the BH domain of the reference protein sequence.

50. The Type II Cas protein of any one of embodiments 1 to 46, wherein the amino acid sequence of the Type II Cas protein comprises an amino acid sequence that is at least 65% identical to the amino acid sequence of the BH domain of the reference protein sequence.

51. The Type II Cas protein of any one of embodiments 1 to 46, wherein the amino acid sequence of the Type II Cas protein comprises an amino acid sequence that is at least 70% identical to the amino acid sequence of the BH domain of the reference protein sequence.

52. The Type II Cas protein of any one of embodiments 1 to 46, wherein the amino acid sequence of the Type II Cas protein comprises an amino acid sequence that is at least 75% identical to the amino acid sequence of the BH domain of the reference protein sequence.

53. The Type II Cas protein of any one of embodiments 1 to 46, wherein the amino acid sequence of the Type II Cas protein comprises an amino acid sequence that is at least 80% identical to the amino acid sequence of the BH domain of the reference protein sequence.

54. The Type II Cas protein of any one of embodiments 1 to 46, wherein the amino acid sequence of the Type II Cas protein comprises an amino acid sequence that is at least 85% identical to the amino acid sequence of the BH domain of the reference protein sequence.

55. The Type II Cas protein of any one of embodiments 1 to 46, wherein the amino acid sequence of the Type II Cas protein comprises an amino acid sequence that is at least 90% identical to the amino acid sequence of the BH domain of the reference protein sequence.

56. The Type II Cas protein of any one of embodiments 1 to 46, wherein the amino acid sequence of the Type II Cas protein comprises an amino acid sequence that is at least 95% identical to the amino acid sequence of the BH domain of the reference protein sequence.

57. The Type II Cas protein of any one of embodiments 1 to 46, wherein the amino acid sequence of the Type II Cas protein comprises an amino acid sequence that is at least 96% identical to the amino acid sequence of the BH domain of the reference protein sequence.

58. The Type II Cas protein of any one of embodiments 1 to 46, wherein the amino acid sequence of the Type II Cas protein comprises an amino acid sequence that is at least 97% identical to the amino acid sequence of the BH domain of the reference protein sequence.

59. The Type II Cas protein of any one of embodiments 1 to 46, wherein the amino acid sequence of the Type II Cas protein comprises an amino acid sequence that is at least 98% identical to the amino acid sequence of the BH domain of the reference protein sequence.

60. The Type II Cas protein of any one of embodiments 1 to 46, wherein the amino acid sequence of the Type II Cas protein comprises an amino acid sequence that is at least 99% identical to the amino acid sequence of the BH domain of the reference protein sequence.

61. The Type II Cas protein of any one of embodiments 1 to 46, wherein the amino acid sequence of the Type II Cas protein comprises an amino acid sequence that is identical to the amino acid sequence of the BH domain of the reference protein sequence.

62. The Type II Cas protein of any one of embodiments 1 to 61, wherein the amino acid sequence of the Type II Cas protein comprises an amino acid sequence that is at least 50% identical to the amino acid sequence of the REC domain of the reference protein sequence.

63. The Type II Cas protein of any one of embodiments 1 to 61, wherein the amino acid sequence of the Type II Cas protein comprises an amino acid sequence that is at least 55% identical to the amino acid sequence of the REC domain of the reference protein sequence.

64. The Type II Cas protein of any one of embodiments 1 to 61, wherein the amino acid sequence of the Type II Cas protein comprises an amino acid sequence that is at least 60% identical to the amino acid sequence of the REC domain of the reference protein sequence.

65. The Type II Cas protein of any one of embodiments 1 to 61, wherein the amino acid sequence of the Type II Cas protein comprises an amino acid sequence that is at least 65% identical to the amino acid sequence of the REC domain of the reference protein sequence.

66. The Type II Cas protein of any one of embodiments 1 to 61, wherein the amino acid sequence of the Type II Cas protein comprises an amino acid sequence that is at least 70% identical to the amino acid sequence of the REC domain of the reference protein sequence.

67. The Type II Cas protein of any one of embodiments 1 to 61, wherein the amino acid sequence of the Type II Cas protein comprises an amino acid sequence that is at least 75% identical to the amino acid sequence of the REC domain of the reference protein sequence.

68. The Type II Cas protein of any one of embodiments 1 to 61, wherein the amino acid sequence of the Type II Cas protein comprises an amino acid sequence that is at least 80% identical to the amino acid sequence of the REC domain of the reference protein sequence.

69. The Type II Cas protein of any one of embodiments 1 to 61, wherein the amino acid sequence of the Type II Cas protein comprises an amino acid sequence that is at least 85% identical to the amino acid sequence of the REC domain of the reference protein sequence.

70. The Type II Cas protein of any one of embodiments 1 to 61, wherein the amino acid sequence of the Type II Cas protein comprises an amino acid sequence that is at least 90% identical to the amino acid sequence of the REC domain of the reference protein sequence.

71. The Type II Cas protein of any one of embodiments 1 to 61, wherein the amino acid sequence of the Type II Cas protein comprises an amino acid sequence that is at least 95% identical to the amino acid sequence of the REC domain of the reference protein sequence.

72. The Type II Cas protein of any one of embodiments 1 to 61, wherein the amino acid sequence of the Type II Cas protein comprises an amino acid sequence that is at least 96% identical to the amino acid sequence of the REC domain of the reference protein sequence.

73. The Type II Cas protein of any one of embodiments 1 to 61, wherein the amino acid sequence of the Type II Cas protein comprises an amino acid sequence that is at least 97% identical to the amino acid sequence of the REC domain of the reference protein sequence.

74. The Type II Cas protein of any one of embodiments 1 to 61, wherein the amino acid sequence of the Type II Cas protein comprises an amino acid sequence that is at least 98% identical to the amino acid sequence of the REC domain of the reference protein sequence.

75. The Type II Cas protein of any one of embodiments 1 to 61, wherein the amino acid sequence of the Type II Cas protein comprises an amino acid sequence that is at least 99% identical to the amino acid sequence of the REC domain of the reference protein sequence.

76. The Type II Cas protein of any one of embodiments 1 to 61, wherein the amino acid sequence of the Type II Cas protein comprises an amino acid sequence that is identical to the amino acid sequence of the REC domain of the reference protein sequence.

77. The Type II Cas protein of any one of embodiments 1 to 76, wherein the amino acid sequence of the Type II Cas protein comprises an amino acid sequence that is at least 50% identical to the amino acid sequence of the HNH domain of the reference protein sequence.

78. The Type II Cas protein of any one of embodiments 1 to 76, wherein the amino acid sequence of the Type II Cas protein comprises an amino acid sequence that is at least 55% identical to the amino acid sequence of the HNH domain of the reference protein sequence.

79. The Type II Cas protein of any one of embodiments 1 to 76, wherein the amino acid sequence of the Type II Cas protein comprises an amino acid sequence that is at least 60% identical to the amino acid sequence of the HNH domain of the reference protein sequence.

80. The Type II Cas protein of any one of embodiments 1 to 76, wherein the amino acid sequence of the Type II Cas protein comprises an amino acid sequence that is at least 65% identical to the amino acid sequence of the HNH domain of the reference protein sequence.

81. The Type II Cas protein of any one of embodiments 1 to 76, wherein the amino acid sequence of the Type II Cas protein comprises an amino acid sequence that is at least 70% identical to the amino acid sequence of the HNH domain of the reference protein sequence.

82. The Type II Cas protein of any one of embodiments 1 to 76, wherein the amino acid sequence of the Type II Cas protein comprises an amino acid sequence that is at least 75% identical to the amino acid sequence of the HNH domain of the reference protein sequence.

83. The Type II Cas protein of any one of embodiments 1 to 76, wherein the amino acid sequence of the Type II Cas protein comprises an amino acid sequence that is at least 80% identical to the amino acid sequence of the HNH domain of the reference protein sequence.

84. The Type II Cas protein of any one of embodiments 1 to 76, wherein the amino acid sequence of the Type II Cas protein comprises an amino acid sequence that is at least 85% identical to the amino acid sequence of the HNH domain of the reference protein sequence.

85. The Type II Cas protein of any one of embodiments 1 to 76, wherein the amino acid sequence of the Type II Cas protein comprises an amino acid sequence that is at least 90% identical to the amino acid sequence of the HNH domain of the reference protein sequence.

86. The Type II Cas protein of any one of embodiments 1 to 76, wherein the amino acid sequence of the Type II Cas protein comprises an amino acid sequence that is at least 95% identical to the amino acid sequence of the HNH domain of the reference protein sequence.

87. The Type II Cas protein of any one of embodiments 1 to 76, wherein the amino acid sequence of the Type II Cas protein comprises an amino acid sequence that is at least 96% identical to the amino acid sequence of the HNH domain of the reference protein sequence.

88. The Type II Cas protein of any one of embodiments 1 to 76, wherein the amino acid sequence of the Type II Cas protein comprises an amino acid sequence that is at least 97% identical to the amino acid sequence of the HNH domain of the reference protein sequence.

89. The Type II Cas protein of any one of embodiments 1 to 76, wherein the amino acid sequence of the Type II Cas protein comprises an amino acid sequence that is at least 98% identical to the amino acid sequence of the HNH domain of the reference protein sequence.

90. The Type II Cas protein of any one of embodiments 1 to 76, wherein the amino acid sequence of the Type II Cas protein comprises an amino acid sequence that is at least 99% identical to the amino acid sequence of the HNH domain of the reference protein sequence.

91. The Type II Cas protein of any one of embodiments 1 to 76, wherein the amino acid sequence of the Type II Cas protein comprises an amino acid sequence that is identical to the amino acid sequence of the HNH domain of the reference protein sequence.

92. The Type II Cas protein of any one of embodiments 1 to 91, wherein the amino acid sequence of the Type II Cas protein comprises an amino acid sequence that is at least 50% identical to the amino acid sequence of the WED domain of the reference protein sequence.

93. The Type II Cas protein of any one of embodiments 1 to 91, wherein the amino acid sequence of the Type II Cas protein comprises an amino acid sequence that is at least 55% identical to the amino acid sequence of the WED domain of the reference protein sequence.

94. The Type II Cas protein of any one of embodiments 1 to 91, wherein the amino acid sequence of the Type II Cas protein comprises an amino acid sequence that is at least 60% identical to the amino acid sequence of the WED domain of the reference protein sequence.

95. The Type II Cas protein of any one of embodiments 1 to 91, wherein the amino acid sequence of the Type II Cas protein comprises an amino acid sequence that is at least 65% identical to the amino acid sequence of the WED domain of the reference protein sequence.

96. The Type II Cas protein of any one of embodiments 1 to 91, wherein the amino acid sequence of the Type II Cas protein comprises an amino acid sequence that is at least 70% identical to the amino acid sequence of the WED domain of the reference protein sequence.

97. The Type II Cas protein of any one of embodiments 1 to 91, wherein the amino acid sequence of the Type II Cas protein comprises an amino acid sequence that is at least 75% identical to the amino acid sequence of the WED domain of the reference protein sequence.

98. The Type II Cas protein of any one of embodiments 1 to 91, wherein the amino acid sequence of the Type II Cas protein comprises an amino acid sequence that is at least 80% identical to the amino acid sequence of the WED domain of the reference protein sequence.

99. The Type II Cas protein of any one of embodiments 1 to 91, wherein the amino acid sequence of the Type II Cas protein comprises an amino acid sequence that is at least 85% identical to the amino acid sequence of the WED domain of the reference protein sequence.

100. The Type II Cas protein of any one of embodiments 1 to 91, wherein the amino acid sequence of the Type II Cas protein comprises an amino acid sequence that is at least 90% identical to the amino acid sequence of the WED domain of the reference protein sequence.

101. The Type II Cas protein of any one of embodiments 1 to 91, wherein the amino acid sequence of the Type II Cas protein comprises an amino acid sequence that is at least 95% identical to the amino acid sequence of the WED domain of the reference protein sequence.

102. The Type II Cas protein of any one of embodiments 1 to 91, wherein the amino acid sequence of the Type II Cas protein comprises an amino acid sequence that is at least 96% identical to the amino acid sequence of the WED domain of the reference protein sequence.

103. The Type II Cas protein of any one of embodiments 1 to 91, wherein the amino acid sequence of the Type II Cas protein comprises an amino acid sequence that is at least 97% identical to the amino acid sequence of the WED domain of the reference protein sequence.

104. The Type II Cas protein of any one of embodiments 1 to 91, wherein the amino acid sequence of the Type II Cas protein comprises an amino acid sequence that is at least 98% identical to the amino acid sequence of the WED domain of the reference protein sequence.

105. The Type II Cas protein of any one of embodiments 1 to 91, wherein the amino acid sequence of the Type II Cas protein comprises an amino acid sequence that is at least 99% identical to the amino acid sequence of the WED domain of the reference protein sequence.

106. The Type II Cas protein of any one of embodiments 1 to 91, wherein the amino acid sequence of the Type II Cas protein comprises an amino acid sequence that is identical to the amino acid sequence of the WED domain of the reference protein sequence.

107. The Type II Cas protein of any one of embodiments 1 to 106, wherein the amino acid sequence of the Type II Cas protein comprises an amino acid sequence that is at least 50% identical to the amino acid sequence of the PID domain of the reference protein sequence.

108. The Type II Cas protein of any one of embodiments 1 to 106, wherein the amino acid sequence of the Type II Cas protein comprises an amino acid sequence that is at least 55% identical to the amino acid sequence of the PID domain of the reference protein sequence.

109. The Type II Cas protein of any one of embodiments 1 to 106, wherein the amino acid sequence of the Type II Cas protein comprises an amino acid sequence that is at least 60% identical to the amino acid sequence of the PID domain of the reference protein sequence.

110. The Type II Cas protein of any one of embodiments 1 to 106, wherein the amino acid sequence of the Type II Cas protein comprises an amino acid sequence that is at least 65% identical to the amino acid sequence of the PID domain of the reference protein sequence.

111. The Type II Cas protein of any one of embodiments 1 to 106, wherein the amino acid sequence of the Type II Cas protein comprises an amino acid sequence that is at least 70% identical to the amino acid sequence of the PID domain of the reference protein sequence.

112. The Type II Cas protein of any one of embodiments 1 to 106, wherein the amino acid sequence of the Type II Cas protein comprises an amino acid sequence that is at least 75% identical to the amino acid sequence of the PID domain of the reference protein sequence.

113. The Type II Cas protein of any one of embodiments 1 to 106, wherein the amino acid sequence of the Type II Cas protein comprises an amino acid sequence that is at least 80% identical to the amino acid sequence of the PID domain of the reference protein sequence.

114. The Type II Cas protein of any one of embodiments 1 to 106, wherein the amino acid sequence of the Type II Cas protein comprises an amino acid sequence that is at least 85% identical to the amino acid sequence of the PID domain of the reference protein sequence.

115. The Type II Cas protein of any one of embodiments 1 to 106, wherein the amino acid sequence of the Type II Cas protein comprises an amino acid sequence that is at least 90% identical to the amino acid sequence of the PID domain of the reference protein sequence.

116. The Type II Cas protein of any one of embodiments 1 to 106, wherein the amino acid sequence of the Type II Cas protein comprises an amino acid sequence that is at least 95% identical to the amino acid sequence of the PID domain of the reference protein sequence.

117. The Type II Cas protein of any one of embodiments 1 to 106, wherein the amino acid sequence of the Type II Cas protein comprises an amino acid sequence that is at least 96% identical to the amino acid sequence of the PID domain of the reference protein sequence.

118. The Type II Cas protein of any one of embodiments 1 to 106, wherein the amino acid sequence of the Type II Cas protein comprises an amino acid sequence that is at least 97% identical to the amino acid sequence of the PID domain of the reference protein sequence.

119. The Type II Cas protein of any one of embodiments 1 to 106, wherein the amino acid sequence of the Type II Cas protein comprises an amino acid sequence that is at least 98% identical to the amino acid sequence of the PID domain of the reference protein sequence.

120. The Type II Cas protein of any one of embodiments 1 to 106, wherein the amino acid sequence of the Type II Cas protein comprises an amino acid sequence that is at least 99% identical to the amino acid sequence of the PID domain of the reference protein sequence.

121. The Type II Cas protein of any one of embodiments 1 to 106, wherein the amino acid sequence of the Type II Cas protein comprises an amino acid sequence that is identical to the amino acid sequence of the PID domain of the reference protein sequence.

122. The Type II Cas protein of embodiment 1, wherein the amino acid sequence of the Type II Cas protein comprises an amino acid sequence that is at least 55% identical to the full length of the reference protein sequence.

123. The Type II Cas protein of embodiment 1, wherein the amino acid sequence of the Type II Cas protein comprises an amino acid sequence that is at least 60% identical to the full length of the reference protein sequence.

124. The Type II Cas protein of embodiment 1, wherein the amino acid sequence of the Type II Cas protein comprises an amino acid sequence that is at least 65% identical to the full length of the reference protein sequence.

125. The Type II Cas protein of embodiment 1, wherein the amino acid sequence of the Type II Cas protein comprises an amino acid sequence that is at least 70% identical to the full length of the reference protein sequence.

126. The Type II Cas protein of embodiment 1, wherein the amino acid sequence of the Type II Cas protein comprises an amino acid sequence that is at least 75% identical to the full length of the reference protein sequence.

127. The Type II Cas protein of embodiment 1, wherein the amino acid sequence of the Type II Cas protein comprises an amino acid sequence that is at least 80% identical to the full length of the reference protein sequence.

128. The Type II Cas protein of embodiment 1, wherein the amino acid sequence of the Type II Cas protein comprises an amino acid sequence that is at least 85% identical to the full length of the reference protein sequence.

129. The Type II Cas protein of embodiment 1, wherein the amino acid sequence of the Type II Cas protein comprises an amino acid sequence that is at least 90% identical to the full length of the reference protein sequence.

130. The Type II Cas protein of embodiment 1, wherein the amino acid sequence of the Type II Cas protein comprises an amino acid sequence that is at least 95% identical to the full length of the reference protein sequence.

131. The Type II Cas protein of embodiment 1, wherein the amino acid sequence of the Type II Cas protein comprises an amino acid sequence that is at least 96% identical to the full length of the reference protein sequence.

132. The Type II Cas protein of embodiment 1, wherein the amino acid sequence of the Type II Cas protein comprises an amino acid sequence that is at least 97% identical to the full length of the reference protein sequence.

133. The Type II Cas protein of embodiment 1, wherein the amino acid sequence of the Type II Cas protein comprises an amino acid sequence that is at least 98% identical to the full length of the reference protein sequence.

134. The Type II Cas protein of embodiment 1, wherein the amino acid sequence of the Type II Cas protein comprises an amino acid sequence that is at least 99% identical to the full length of the reference protein sequence.

135. The Type II Cas protein of embodiment 1, wherein the amino acid sequence of the Type II Cas protein comprises an amino acid sequence that is identical to the full length of the reference protein sequence.

136. The Type II Cas protein of any one of embodiments 1 to 134, which is a chimeric Type II Cas protein.

137. The Type II Cas protein of any one of embodiments 1 to 136, which is a fusion protein.

138. The Type II Cas protein of embodiment 137, which comprises one or more nuclear localization signals.

139. The Type II Cas protein of embodiment 138, which comprises two or more nuclear localization signals.

140. The Type II Cas protein of embodiment 138 or embodiment 139, which comprises an N-terminal nuclear localization signal.

141. The Type II Cas protein of any one of embodiments 138 to 140, which comprises a C-terminal nuclear localization signal.

142. The Type II Cas protein of any one of embodiments 138 to 141, which comprises an N-terminal nuclear localization signal and a C-terminal nuclear localization signal.

143. The Type II Cas protein of any one of embodiments 138 to 142, wherein the amino acid sequence of one or more of the nuclear localization signals comprises the amino acid sequence

(SEQ ID NO: 109)
KRTADGSEFESPKKKRKV,
(SEQ ID NO: 110)
PKKKRKV,
(SEQ ID NO: 111)
PKKKRRV,
(SEQ ID NO: 112)
KRPAATKKAGQAKKKK,
(SEQ ID NO: 113)
YGRKKRRQRRR,
(SEQ ID NO: 114)
RKKRRQRRR,
(SEQ ID NO: 115)
PAAKRVKLD,
(SEQ ID NO: 116)
RQRRNELKRSP,
(SEQ ID NO: 117)
VSRKRPRP,
(SEQ ID NO: 118)
PPKKARED,
(SEQ ID NO: 119)
PQPKKKPL,
(SEQ ID NO: 120)
SALIKKKKKMAP,
(SEQ ID NO: 121)
PKQKKRK,
(SEQ ID NO: 122)
RKLKKKIKKL,
(SEQ ID NO: 123)
REKKKFLKRR,
(SEQ ID NO: 124)
KRKGDEVDGVDEVAKKKSKK,
(SEQ ID NO: 125)
RKCLQAGMNLEARKTKK,
(SEQ ID NO: 126)
NQSSNFGPMKGGNFGGRSSGPYGGGGQYFAKPRNQGGY,
or
(SEQ ID NO: 127)
RMRIZFKNKGKDTAELRRRRVEVSVELRKAKKDEQILKRRNV.

144. The Type II Cas protein of embodiment 143, wherein the amino acid sequence of one or more of the nuclear localization signals comprises the amino acid sequence KRTADGSEFESPKKKRKV (SEQ ID NO:109).

145. The Type II Cas protein of embodiment 143, wherein the amino acid sequence of one or more of the nuclear localization signals comprises the amino acid sequence PKKKRKV (SEQ ID NO:110).

146. The Type II Cas protein of embodiment 143, wherein the amino acid sequence of one or more of the nuclear localization signals comprises the amino acid sequence PKKKRRV (SEQ ID NO:111).

147. The Type II Cas protein of embodiment 143, wherein the amino acid sequence of one or more of the nuclear localization signals comprises the amino acid sequence KRPAATKKAGQAKKKK (SEQ ID NO:112).

148. The Type II Cas protein of embodiment 143, wherein the amino acid sequence of one or more of the nuclear localization signals comprises the amino acid sequence YGRKKRRQRRR (SEQ ID NO:113).

149. The Type II Cas protein of embodiment 143, wherein the amino acid sequence of one or more of the nuclear localization signals comprises the amino acid sequence RKKRRQRRR (SEQ ID NO:114).

150. The Type II Cas protein of embodiment 143, wherein the amino acid sequence of one or more of the nuclear localization signals comprises the amino acid sequence PAAKRVKLD (SEQ ID NO:115).

151. The Type II Cas protein of embodiment 143, wherein the amino acid sequence of one or more of the nuclear localization signals comprises the amino acid sequence RQRRNELKRSP (SEQ ID NO:116).

152. The Type II Cas protein of embodiment 143, wherein the amino acid sequence of one or more of the nuclear localization signals comprises the amino acid sequence VSRKRPRP (SEQ ID NO:117).

153. The Type II Cas protein of embodiment 143, wherein the amino acid sequence of one or more of the nuclear localization signals comprises the amino acid sequence PPKKARED (SEQ ID NO:118).

154. The Type II Cas protein of embodiment 143, wherein the amino acid sequence of one or more of the nuclear localization signals comprises the amino acid sequence PQPKKKPL (SEQ ID NO:119).

155. The Type II Cas protein of embodiment 143, wherein the amino acid sequence of one or more of the nuclear localization signals comprises the amino acid sequence SALIKKKKKMAP (SEQ ID NO:120).

156. The Type II Cas protein of embodiment 143, wherein the amino acid sequence of one or more of the nuclear localization signals comprises the amino acid sequence PKQKKRK (SEQ ID NO:121).

157. The Type II Cas protein of embodiment 143, wherein the amino acid sequence of one or more of the nuclear localization signals comprises the amino acid sequence RKLKKKIKKL (SEQ ID NO:122).

158. The Type II Cas protein of embodiment 143, wherein the amino acid sequence of one or more of the nuclear localization signals comprises the amino acid sequence REKKKFLKRR (SEQ ID NO:123).

159. The Type II Cas protein of embodiment 143, wherein the amino acid sequence of one or more of the nuclear localization signals comprises the amino acid sequence

(SEQ ID NO: 124)
KRKGDEVDGVDEVAKKKSKK.

160. The Type II Cas protein of embodiment 143, wherein the amino acid sequence of one or more of the nuclear localization signals comprises the amino acid sequence RKCLQAGMNLEARKTKK (SEQ ID NO:125).

161. The Type II Cas protein of embodiment 143, wherein the amino acid sequence of one or more of the nuclear localization signals comprises the amino acid sequence

(SEQ ID NO: 126)
NQSSNFGPMKGGNFGGRSSGPYGGGGQYFAKPRNQGGY.

162. The Type II Cas protein of embodiment 143, wherein the amino acid sequence of one or more of the nuclear localization signals comprises the amino acid sequence

(SEQ ID NO: 127)
RMRIZFKNKGKDTAELRRRRVEVSVELRKAKKDEQILKRRNV.

163. The Type II Cas protein of any one of embodiments 138 to 162, wherein the amino acid sequence of each nuclear localization signal is the same.

164. The Type II Cas protein of any one of embodiments 136 to 163, which comprises a fusion partner which is a DNA, RNA or protein modification enzyme, optionally wherein the DNA, RNA or protein modification enzyme is an adenosine deaminase, a cytidine deaminase, a reverse transcriptase, a guanosyl transferase, a DNA methyltransferase, a RNA methyltransferase, a DNA demethylase, a RNA demethylase, a dioxygenase, a polyadenylate polymerase, a pseudouridine synthase, an acetyltransferase, a deacetylase, a ubiquitin-ligase, a deubiquitinase, a kinase, a phosphatase, a NEDD8-ligase, a de-NEDDylase, a SUMO-ligase, a deSUMOylase, a histone deacetylase, a histone acetyltransferase, a histone methyltransferase, or a histone demethylase.

165. The Type II Cas protein of any one of embodiments 136 to 164, which comprises a means for deaminating adenosine, optionally wherein the means for deaminating adenosine is an adenosine deaminase.

166. The Type II Cas protein of any one of embodiments 136 to 164, which comprises a fusion partner which is an adenosine deaminase, optionally wherein the amino acid sequence of the adenosine deaminase comprises an amino acid sequence having at least 50%, at least 60%, at least 70%, at least 80%, at least 90%, at least 95%, at least 97%, at least 98%, at least 99%, or 100% sequence identity with SEQ ID NO:130, optionally wherein the adenosine deaminase is the adenosine deaminase moiety contained in the adenine base editor ABE8e.

167. The Type II Cas protein of any one of embodiments 136 to 164, which comprises a means for deaminating cytidine, optionally wherein the means for deaminating cytidine is a cytidine deaminase.

168. The Type II Cas protein of any one of embodiments 136 to 164, which comprises a fusion partner which is a cytidine deaminase.

169. The Type II Cas protein of any one of embodiments 136 to 164, which comprises a means for synthesizing DNA from a single-stranded template, optionally wherein the means for synthesizing DNA from a single-stranded template is a reverse transcriptase.

170. The Type II Cas protein of any one of embodiments 136 to 164, which comprises a fusion partner which is a reverse transcriptase.

171. The Type II Cas protein of any one of embodiments 136 to 170, which comprises a tag.

172. The Type II Cas protein of embodiment 171, wherein the tag is a SV5 tag, optionally wherein the SV5 tag comprises the amino acid sequence GKPIPNPLLGLDST (SEQ ID NO:128) or IPNPLLGLD (SEQ ID NO:129).

173. The Type II Cas protein of any one of embodiments 1 to 172, wherein the reference protein sequence is SEQ ID NO:1.

174. The Type II Cas protein of embodiment 173, whose amino acid sequence comprises the amino acid sequence of SEQ ID NO:1.

175. The Type II Cas protein of any one of embodiments 1 to 172, wherein the reference protein sequence is SEQ ID NO:2.

176. The Type II Cas protein of any one of embodiments 173 to 175, whose amino acid sequence comprises the amino acid sequence of SEQ ID NO:2.

177. The Type II Cas protein of embodiment 173 or embodiment 174, whose amino acid sequence comprises the amino acid sequence of SEQ ID NO:3.

178. The Type II Cas protein of any one of embodiments 1 to 172, wherein the reference protein sequence is SEQ ID NO:7.

179. The Type II Cas protein of embodiment 178, whose amino acid sequence comprises the amino acid sequence of SEQ ID NO:7.

180. The Type II Cas protein of any one of embodiments 1 to 172, wherein the reference protein sequence is SEQ ID NO:8.

181. The Type II Cas protein of any one of embodiments 178 to 180, whose amino acid sequence comprises the amino acid sequence of SEQ ID NO:8.

182. The Type II Cas protein of embodiment 178 or embodiment 179, whose amino acid sequence comprises the amino acid sequence of SEQ ID NO:9.

183. The Type II Cas protein of any one of embodiments 1 to 172, wherein the reference protein sequence is SEQ ID NO:13.

184. The Type II Cas protein of embodiment 183, whose amino acid sequence comprises the amino acid sequence of SEQ ID NO:13.

185. The Type II Cas protein of any one of embodiments 1 to 172, wherein the reference protein sequence is SEQ ID NO:14.

186. The Type II Cas protein of any one of embodiments 183 to 185, whose amino acid sequence comprises the amino acid sequence of SEQ ID NO:14.

187. The Type II Cas protein of embodiment 183 or embodiment 184, whose amino acid sequence comprises the amino acid sequence of SEQ ID NO:15.

188. The Type II Cas protein of any one of embodiments 1 to 172, wherein the reference protein sequence is SEQ ID NO:19.

189. The Type II Cas protein of embodiment 188, whose amino acid sequence comprises the amino acid sequence of SEQ ID NO:19.

190. The Type II Cas protein of any one of embodiments 1 to 172, wherein the reference protein sequence is SEQ ID NO:20.

191. The Type II Cas protein of any one of embodiments 188 to 190, whose amino acid sequence comprises the amino acid sequence of SEQ ID NO:20.

192. The Type II Cas protein of embodiment 188 or embodiment 189, whose amino acid sequence comprises the amino acid sequence of SEQ ID NO:21.

193. The Type II Cas protein of any one of embodiments 1 to 172, wherein the reference protein sequence is SEQ ID NO:25.

194. The Type II Cas protein of embodiment 193, whose amino acid sequence comprises the amino acid sequence of SEQ ID NO:25.

195. The Type II Cas protein of any one of embodiments 1 to 172, wherein the reference protein sequence is SEQ ID NO:26.

196. The Type II Cas protein of any one of embodiments 193 to 195, whose amino acid sequence comprises the amino acid sequence of SEQ ID NO:26.

197. The Type II Cas protein of embodiment 194 or embodiment 195, whose amino acid sequence comprises the amino acid sequence of SEQ ID NO:27.

198. The Type II Cas protein of any one of embodiments 1 to 172, wherein the reference protein sequence is SEQ ID NO:31.

199. The Type II Cas protein of embodiment 198, whose amino acid sequence comprises the amino acid sequence of SEQ ID NO:31.

200. The Type II Cas protein of any one of embodiments 1 to 172, wherein the reference protein sequence is SEQ ID NO:32.

201. The Type II Cas protein of any one of embodiments 199 to 200, whose amino acid sequence comprises the amino acid sequence of SEQ ID NO:32.

202. The Type II Cas protein of embodiment 198 or embodiment 199, whose amino acid sequence comprises the amino acid sequence of SEQ ID NO:33.

203. The Type II Cas protein of any one of embodiments 1 to 172, wherein the reference protein sequence is SEQ ID NO:37.

204. The Type II Cas protein of embodiment 203, whose amino acid sequence comprises the amino acid sequence of SEQ ID NO:37.

205. The Type II Cas protein of any one of embodiments 1 to 172, wherein the reference protein sequence is SEQ ID NO:38.

206. The Type II Cas protein of any one of embodiments 203 to 205, whose amino acid sequence comprises the amino acid sequence of SEQ ID NO:38.

207. The Type II Cas protein of embodiment 203 or embodiment 204, whose amino acid sequence comprises the amino acid sequence of SEQ ID NO:39.

208. The Type II Cas protein of any one of embodiments 1 to 172, wherein the reference protein sequence is SEQ ID NO:43.

209. The Type II Cas protein of embodiment 208, whose amino acid sequence comprises the amino acid sequence of SEQ ID NO:43.

210. The Type II Cas protein of any one of embodiments 1 to 172, wherein the reference protein sequence is SEQ ID NO:44.

211. The Type II Cas protein of any one of embodiments 208 to 210, whose amino acid sequence comprises the amino acid sequence of SEQ ID NO:44.

212. The Type II Cas protein of embodiment 208 or embodiment 209, whose amino acid sequence comprises the amino acid sequence of SEQ ID NO:45.

213. The Type II Cas protein of any one of embodiments 1 to 172, wherein the reference protein sequence is SEQ ID NO:49.

214. The Type II Cas protein of embodiment 213, whose amino acid sequence comprises the amino acid sequence of SEQ ID NO:49.

215. The Type II Cas protein of any one of embodiments 1 to 172, wherein the reference protein sequence is SEQ ID NO:50.

216. The Type II Cas protein of any one of embodiments 213 to 215, whose amino acid sequence comprises the amino acid sequence of SEQ ID NO:50.

217. The Type II Cas protein of embodiment 213 or embodiment 214, whose amino acid sequence comprises the amino acid sequence of SEQ ID NO:51.

218. The Type II Cas protein of any one of embodiments 1 to 172, wherein the reference protein sequence is SEQ ID NO:55.

219. The Type II Cas protein of embodiment 218, whose amino acid sequence comprises the amino acid sequence of SEQ ID NO:55.

220. The Type II Cas protein of any one of embodiments 1 to 172, wherein the reference protein sequence is SEQ ID NO:56.

221. The Type II Cas protein of any one of embodiments 218 to 220, whose amino acid sequence comprises the amino acid sequence of SEQ ID NO:56.

222. The Type II Cas protein of embodiment 218 or embodiment 219, whose amino acid sequence comprises the amino acid sequence of SEQ ID NO:57.

223. A Type II Cas protein whose amino acid sequence is identical to a Type II Cas protein of any one of embodiments 1 to 222 except for one or more amino acid substitutions relative to the reference sequence that provide nickase activity.

224. The Type II Cas of embodiment 223, wherein the one or more amino acid substitutions that provide nickase activity are in a RuvC or HNH domain.

225. The Type II Cas of embodiment 223, wherein the one or more amino acid substitutions relative to the reference sequence that provide nickase activity comprise a D13A substitution, wherein the position of the D13A substitution is defined with respect to the amino acid numbering of SEQ ID NO:2.

226. The Type II Cas of embodiment 223, wherein the one or more amino acid substitutions relative to the reference sequence that provide nickase activity comprise a N589A substitution, wherein the position of the N589A substitution is defined with respect to the amino acid numbering of SEQ ID NO:2.

227. The Type II Cas of embodiment 223, wherein the one or more amino acid substitutions relative to the reference sequence that provide nickase activity comprise a D8A substitution, wherein the position of the D8A substitution is defined with respect to the amino acid numbering of SEQ ID NO:8.

228. The Type II Cas of embodiment 223, wherein the one or more amino acid substitutions relative to the reference sequence that provide nickase activity comprise a N587A substitution, wherein the position of the N587A substitution is defined with respect to the amino acid numbering of SEQ ID NO:8.

229. The Type II Cas of embodiment 223, wherein the one or more amino acid substitutions relative to the reference sequence that provide nickase activity comprise a D9A substitution, wherein the position of the D9A substitution is defined with respect to the amino acid numbering of SEQ ID NO:14.

230. The Type II Cas of embodiment 223, wherein the one or more amino acid substitutions relative to the reference sequence that provide nickase activity comprise a N590A substitution, wherein the position of the N590A substitution is defined with respect to the amino acid numbering of SEQ ID NO:14.

231. The Type II Cas of embodiment 223, wherein the one or more amino acid substitutions relative to the reference sequence that provide nickase activity comprise a D6A substitution, wherein the position of the D6A substitution is defined with respect to the amino acid numbering of SEQ ID NO:20.

232. The Type II Cas of embodiment 223, wherein the one or more amino acid substitutions relative to the reference sequence that provide nickase activity comprise a N611A substitution, wherein the position of the N611A substitution is defined with respect to the amino acid numbering of SEQ ID NO:20.

233. The Type II Cas of embodiment 223, wherein the one or more amino acid substitutions relative to the reference sequence that provide nickase activity comprise a D9A substitution, wherein the position of the D9A substitution is defined with respect to the amino acid numbering of SEQ ID NO:26.

234. The Type II Cas of embodiment 223, wherein the one or more amino acid substitutions relative to the reference sequence that provide nickase activity comprise a N590A substitution, wherein the position of the N590A substitution is defined with respect to the amino acid numbering of SEQ ID NO:26.

235. The Type II Cas of embodiment 223, wherein the one or more amino acid substitutions relative to the reference sequence that provide nickase activity comprise a D9A substitution, wherein the position of the D9A substitution is defined with respect to the amino acid numbering of SEQ ID NO:32.

236. The Type II Cas of embodiment 223, wherein the one or more amino acid substitutions relative to the reference sequence that provide nickase activity comprise a N590A substitution, wherein the position of the N590A substitution is defined with respect to the amino acid numbering of SEQ ID NO:32.

237. The Type II Cas of embodiment 223, wherein the one or more amino acid substitutions relative to the reference sequence that provide nickase activity comprise a D9A substitution, wherein the position of the D9A substitution is defined with respect to the amino acid numbering of SEQ ID NO:38.

238. The Type II Cas of embodiment 223, wherein the one or more amino acid substitutions relative to the reference sequence that provide nickase activity comprise a N590A substitution, wherein the position of the N590A substitution is defined with respect to the amino acid numbering of SEQ ID NO:38.

239. The Type II Cas of embodiment 223, wherein the one or more amino acid substitutions relative to the reference sequence that provide nickase activity comprise a D23A substitution, wherein the position of the D23A substitution is defined with respect to the amino acid numbering of SEQ ID NO:44.

240. The Type II Cas of embodiment 223, wherein the one or more amino acid substitutions relative to the reference sequence that provide nickase activity comprise a N629A substitution, wherein the position of the N629A substitution is defined with respect to the amino acid numbering of SEQ ID NO:44.

241. The Type II Cas of embodiment 223, wherein the one or more amino acid substitutions relative to the reference sequence that provide nickase activity comprise a D23A substitution, wherein the position of the D23A substitution is defined with respect to the amino acid numbering of SEQ ID NO:50.

242. The Type II Cas of embodiment 223, wherein the one or more amino acid substitutions relative to the reference sequence that provide nickase activity comprise a N629A substitution, wherein the position of the N629A substitution is defined with respect to the amino acid numbering of SEQ ID NO:50.

243. The Type II Cas of embodiment 223, wherein the one or more amino acid substitutions relative to the reference sequence that provide nickase activity comprise a D23A substitution, wherein the position of the D23A substitution is defined with respect to the amino acid numbering of SEQ ID NO:56.

244. The Type II Cas of embodiment 223, wherein the one or more amino acid substitutions relative to the reference sequence that provide nickase activity comprise a N632A substitution, wherein the position of the N632A substitution is defined with respect to the amino acid numbering of SEQ ID NO:56.

245. A Type II Cas protein whose amino acid sequence is identical to a Type II Cas protein of any one of embodiments 1 to 222 except for one or more amino acid substitutions relative to the reference sequence that render the Type II Cas protein catalytically inactive.

246. The Type II Cas protein of embodiment 245, wherein the one or more amino acid substitutions that render the Type II Cas protein catalytically inactive comprise D13A and N589A substitutions, wherein the positions of the D13A and N589A substitutions are defined with respect to the amino acid numbering of SEQ ID NO:2.

247. The Type II Cas protein of embodiment 245, wherein the one or more amino acid substitutions that render the Type II Cas protein catalytically inactive comprise D8A and N587A substitutions, wherein the positions of the D8A and N587A substitutions are defined with respect to the amino acid numbering of SEQ ID NO:8.

248. The Type II Cas protein of embodiment 245, wherein the one or more amino acid substitutions that render the Type II Cas protein catalytically inactive comprise D9A and N590A substitutions, wherein the positions of the D9A and N590A substitutions are defined with respect to the amino acid numbering of SEQ ID NO:14.

249. The Type II Cas protein of embodiment 245, wherein the one or more amino acid substitutions that render the Type II Cas protein catalytically inactive comprise D6A and N611A substitutions, wherein the positions of the D6A and N611A substitutions are defined with respect to the amino acid numbering of SEQ ID NO:20.

250. The Type II Cas protein of embodiment 245, wherein the one or more amino acid substitutions that render the Type II Cas protein catalytically inactive comprise D9A and N590A substitutions, wherein the positions of the D9A and N590A substitutions are defined with respect to the amino acid numbering of SEQ ID NO:26.

251. The Type II Cas protein of embodiment 245, wherein the one or more amino acid substitutions that render the Type II Cas protein catalytically inactive comprise D9A and N590A substitutions, wherein the positions of the D9A and N590A substitutions are defined with respect to the amino acid numbering of SEQ ID NO:32.

252. The Type II Cas protein of embodiment 245, wherein the one or more amino acid substitutions that render the Type II Cas protein catalytically inactive comprise D9A and N590A substitutions, wherein the positions of the D9A and N590A substitutions are defined with respect to the amino acid numbering of SEQ ID NO:38.

253. The Type II Cas protein of embodiment 245, wherein the one or more amino acid substitutions that render the Type II Cas protein catalytically inactive comprise D23A and N629A substitutions, wherein the positions of the D23A and N629A substitutions are defined with respect to the amino acid numbering of SEQ ID NO:44.

254. The Type II Cas protein of embodiment 245, wherein the one or more amino acid substitutions that render the Type II Cas protein catalytically inactive comprise D23A and N629A substitutions, wherein the positions of the D23A and N629A substitutions are defined with respect to the amino acid numbering of SEQ ID NO:50.

255. The Type II Cas protein of embodiment 245, wherein the one or more amino acid substitutions that render the Type II Cas protein catalytically inactive comprise D23A and N632A substitutions, wherein the positions of the D23A and N632A substitutions are defined with respect to the amino acid numbering of SEQ ID NO:56.

256. An AHZW Type II Cas guide RNA molecule.

257. An ABSE Type II Cas guide RNA molecule.

258. An AIXM Type II Cas guide RNA molecule.

259 An AXTQ Type II Cas guide RNA molecule.

260. An AIWM Type II Cas guide RNA molecule.

261. An AIWR Type II Cas guide RNA molecule.

262. An AIYQ Type II Cas guide RNA molecule.

263 An EQSC Type II Cas guide RNA molecule.

264. A BDLP Type II Cas guide RNA molecule.

265. A BDKL Type II Cas guide RNA molecule.

266. The gRNA of any one of embodiments 256 to 265, which is a gRNA for editing a human RHO gene.

267. The gRNA of any one of embodiments 256 to 265, which is a gRNA for editing a human B2M gene.

268. The gRNA of any one of embodiments 256 to 265, which is a gRNA for editing a human TRAC gene.

269. The gRNA of any one of embodiments 256 to 265, which is a gRNA for editing a human LAG3 gene.

270. The gRNA of any one of embodiments 256 to 265, which is a gRNA for editing a human PD1 gene.

271. A guide RNA (gRNA) molecule for editing a human RHO gene, the gRNA comprising a spacer whose nucleotide sequence comprises 15 or more consecutive nucleotides of a reference sequence or comprises a nucleotide sequence that is at least 85% identical to the reference sequence, wherein the reference sequence is

(SEQ ID NO: 287)
(a) UUGUGGCUGACCCGYGGCUGCUC;
(SEQ ID NO: 288)
(b) UUGUGGCUGACCCGUGGCUGCUC;
(SEQ ID NO: 289)
(c) UUGUGGCUGACCCGCGGCUGCUC;
(SEQ ID NO: 290)
(d) CUUGUGGCUGACCCGYGGCUGCU;
(SEQ ID NO: 291)
(e) CUUGUGGCUGACCCGUGGCUGCU;
(SEQ ID NO: 292)
(f) CUUGUGGCUGACCCGCGGCUGCU;
(SEQ ID NO: 293)
(g) GGCCCUUGUGGCUGACCCGYGGC;
(SEQ ID NO: 294)
(h) GGCCCUUGUGGCUGACCCGUGGC;
(SEQ ID NO: 295)
(i) GGCCCUUGUGGCUGACCCGCGGC;
(SEQ ID NO: 296)
(j) CUUGGGUGGGAGCAGCCRCGGGU;
(SEQ ID NO: 297)
(k) CUUGGGUGGGAGCAGCCACGGGU;
(SEQ ID NO: 298)
(l) CUUGGGUGGGAGCAGCCGCGGGU;
(SEQ ID NO: 299)
(m) GAGCAGCCRCGGGUCAGCCACAA;
(SEQ ID NO: 300)
(n) GAGCAGCCACGGGUCAGCCACAA;
(SEQ ID NO: 301)
(o) GAGCAGCCGCGGGUCAGCCACAA;
(SEQ ID NO: 302)
(p) CAUGGCUGUGGCCCUUGUGGCUG;
(SEQ ID NO: 303)
(q) GUGGGAGCAGCCRCGGGUCAGCC;
(SEQ ID NO: 304)
(r) GUGGGAGCAGCCACGGGUCAGCC;
(SEQ ID NO: 305)
(s) GUGGGAGCAGCCGCGGGUCAGCC;
(SEQ ID NO: 306)
(t) GGCUGACCCGYGGCUGCUCCCAC;
(SEQ ID NO: 307)
(u) GGCUGACCCGUGGCUGCUCCCAC;
or
(SEQ ID NO: 308)
(v) GGCUGACCCGCGGCUGCUCCCAC ,

where Y is U or C and R is A or G.

272. A guide RNA (gRNA) molecule for editing a human B2M gene, the gRNA comprising a spacer whose nucleotide sequence comprises 15 or more consecutive nucleotides of a reference sequence or comprises a nucleotide sequence that is at least 85% identical to the reference sequence, wherein the reference sequence is

(SEQ ID NO: 309)
(a) CAGAAAGAGAGAGUAGCGCGAGC;
(SEQ ID NO: 310)
(b) CACGUCAUCCAGCAGAGAAUGGA;
(SEQ ID NO: 311)
(c) CAUUCUUCAGUAAGUCAACUUCA;
(SEQ ID NO: 312)
(d) AGCAUUCGGGCCGAGAUGUCUCG;
(SEQ ID NO: 313)
(e) GAGAUGUCUCGCUCCGUGGCCUU;
(SEQ ID NO: 314)
(f) GGAUAGCCUCCAGGCCAGAAAGA;
(SEQ ID NO: 315)
(g) UUGACUUUCCAUUCUCUGCUGGA;
or
(SEQ ID NO: 316)
(h) GGAAAGUCAAAUUUCCUGAAUUG.

273. A guide RNA (gRNA) molecule for editing a human TRAC gene, the gRNA comprising a spacer whose nucleotide sequence comprises 15 or more consecutive nucleotides of a reference sequence or comprises a nucleotide sequence that is at least 85% identical to the reference sequence, wherein the reference sequence is

(SEQ ID NO: 317)
(a) UUUGUCUGUGAUAUACACAUCAG;
(SEQ ID NO: 318)
(b) GUAAGGAUUCUGAUGUGUAUAUCA;
(SEQ ID NO: 319)
(c) GGCCACAGCACUGUUGCUCUUGA;
(SEQ ID NO: 320)
(d) GAGCAACAGUGCUGUGGCCUGGA;
(SEQ ID NO: 321)
(e) GGAGCAACAAAUCUGACUUUGCA;
(SEQ ID NO: 322)
(f) AAUGUGUCACAAAGUAAGGAUUC;
(SEQ ID NO: 323)
(g) UCUGAUGUGUAUAUCACAGACAA;
(SEQ ID NO: 324)
(h) GCCUGGAGCAACAAAUCUGACUUU;
(SEQ ID NO: 325)
(i) GGCGUUUGCACAUGCAAAGUCAG;
(SEQ ID NO: 326)
(j) GAAGAAGGUGUCUUCUGGAAUAA;
or
(SEQ ID NO: 327)
(k) GCUGCCCUUACCUGGGCUGGGGAA.

274. A guide RNA (gRNA) molecule for editing a human PD1 gene, the gRNA comprising a spacer whose nucleotide sequence comprises 15 or more consecutive nucleotides of a reference sequence or comprises a nucleotide sequence that is at least 85% identical to the reference sequence, wherein the reference sequence is

(SEQ ID NO: 328)
(a) UGGGCUGGCGGCCAGGAUGGUUC;
(SEQ ID NO: 329)
(b) CCGCCCAGACGACUGGCCAGGGC;
(SEQ ID NO: 330)
(c) CAGGCGCCCUGGCCAGUCGUCUG;
(SEQ ID NO: 331)
(d) AGAACCAUCCUGGCCGCCAGCCC;
(SEQ ID NO: 332)
(e) CUAAGAACCAUCCUGGCCGCCAG;
(SEQ ID NO: 333)
(f) GCUGGCGGCCAGGAUGGUUCUUAG;
(SEQ ID NO: 334)
(g) ACAACUGGGCUGGCGGCCAGGAU;
(SEQ ID NO: 335)
(h) GUGGGGCUGCUCCAGGCAUGCAG;
or
(SEQ ID NO: 336)
(i) GGCCGCCAGCCCAGUUGUAGCAC.

275. The gRNA of any one of embodiments 271 to 274, which comprises a spacer that is 15 to 30 nucleotides in length.

276. The gRNA of embodiment 275, wherein the spacer is 18 to 30 nucleotides in length.

277. The gRNA of embodiment 275, wherein the spacer is 20 to 28 nucleotides in length.

278. The gRNA of embodiment 275, wherein the spacer is 22 to 26 nucleotides in length.

279. The gRNA of embodiment 275, wherein the spacer is 23 to 25 nucleotides in length.

280. The gRNA of embodiment 275, wherein the spacer is 22 to 25 nucleotides in length.

281. The gRNA of embodiment 275, wherein the spacer is 15 to 25 nucleotides in length.

282. The gRNA of embodiment 275, wherein the spacer is 16 to 24 nucleotides in length.

283. The gRNA of embodiment 275, wherein the spacer is 17 to 23 nucleotides in length.

284. The gRNA of embodiment 275, wherein the spacer is 18 to 22 nucleotides in length.

285. The gRNA of embodiment 275, wherein the spacer is 19 to 21 nucleotides in length.

286. The gRNA of embodiment 275, wherein the spacer is 25 nucleotides in length.

287. The gRNA of embodiment 275, wherein the spacer is 24 nucleotides in length.

288. The gRNA of embodiment 275, wherein the spacer is 23 nucleotides in length.

289. The gRNA of embodiment 275, wherein the spacer is 22 nucleotides in length.

290. The gRNA of embodiment 275, wherein the spacer is 21 nucleotides in length.

291. The gRNA of embodiment 275, wherein the spacer is 20 nucleotides in length.

292. The gRNA of any one of embodiments 271 to 291, wherein the spacer comprises 16 or more consecutive nucleotides of the reference sequence.

293. The gRNA of any one of embodiments 271 to 291, wherein the spacer comprises 17 or more consecutive nucleotides of the reference sequence.

294. The gRNA of any one of embodiments 271 to 291, wherein the spacer comprises 18 or more consecutive nucleotides of the reference sequence.

295. The gRNA of any one of embodiments 271 to 291, wherein the spacer comprises 19 or more consecutive nucleotides of the reference sequence.

296. The gRNA of any one of embodiments 271 to 291, wherein the spacer comprises 20 consecutive nucleotides of the reference sequence.

297. The gRNA of any one of embodiments 271 to 290, wherein the spacer comprises 21 consecutive nucleotides of the reference sequence.

298. The gRNA of any one of embodiments 271 to 289, wherein the spacer comprises 22 consecutive nucleotides of the reference sequence.

299. The gRNA of any one of embodiments 271 to 288, wherein the reference sequence is a reference sequence having at least 23 nucleotides and the spacer comprises 23 consecutive nucleotides of the reference sequence.

300. The gRNA of any one of embodiments 271 to 287, wherein the reference sequence is a reference sequence having at least 24 nucleotides and the spacer comprises 24 consecutive nucleotides of the reference sequence.

301. The gRNA of any one of embodiments 271 to 291, wherein the spacer comprises a nucleotide sequence that is at least 90% identical to the reference sequence.

302. The gRNA of embodiment 301, wherein the spacer comprises a nucleotide sequence that is at least 95% identical to the reference sequence.

303. The gRNA of any one of embodiments 271 to 291, wherein the spacer comprises a nucleotide sequence that has one mismatch relative to the reference sequence.

304. The gRNA of any one of embodiments 271 to 291, wherein the spacer comprises a nucleotide sequence that has two mismatches relative to the reference sequence.

305. The gRNA of any one of embodiments 271 to 291, wherein the spacer comprises the reference sequence.

306. The gRNA of any one of embodiments 271 and 272 to 305 when depending directly or indirectly from embodiment 271, wherein the reference sequence is

(SEQ ID NO: 287)
UUGUGGCUGACCCGYGGCUGCUC.

307. The gRNA of any one of embodiments 271 and 272 to 305 when depending directly or indirectly from embodiment 271, wherein the reference sequence is

(SEQ ID NO: 288)
UUGUGGCUGACCCGUGGCUGCUC.

308. The gRNA of any one of embodiments 271 and 272 to 305 when depending directly or indirectly from embodiment 271, wherein the reference sequence is

(SEQ ID NO: 289)
UUGUGGCUGACCCGCGGCUGCUC.

309. The gRNA of any one of embodiments 271 and 272 to 305 when depending directly or indirectly from embodiment 271, wherein the reference sequence is

(SEQ ID NO: 290)
CUUGUGGCUGACCCGYGGCUGCU.

310. The gRNA of any one of embodiments 271 and 272 to 305 when depending directly or indirectly from embodiment 271, wherein the reference sequence is

(SEQ ID NO: 291)
CUUGUGGCUGACCCGUGGCUGCU.

311. The gRNA of any one of embodiments 271 and 272 to 305 when depending directly or indirectly from embodiment 271, wherein the reference sequence is

(SEQ ID NO: 292)
CUUGUGGCUGACCCGCGGCUGCU.

312. The gRNA of any one of embodiments 271 and 272 to 305 when depending directly or indirectly from embodiment 271, wherein the reference sequence is

(SEQ ID NO: 293)
GGCCCUUGUGGCUGACCCGYGGC.

313. The gRNA of any one of embodiments 271 and 272 to 305 when depending directly or indirectly from embodiment 271, wherein the reference sequence is

(SEQ ID NO: 294)
GGCCCUUGUGGCUGACCCGUGGC.

314. The gRNA of any one of embodiments 271 and 272 to 305 when depending directly or indirectly from embodiment 271, wherein the reference sequence is

(SEQ ID NO: 295)
GGCCCUUGUGGCUGACCCGCGGC.

315. The gRNA of any one of embodiments 271 and 272 to 305 when depending directly or indirectly from embodiment 271, wherein the reference sequence is

(SEQ ID NO: 296)
CUUGGGUGGGAGCAGCCRCGGGU.

316. The gRNA of any one of embodiments 271 and 272 to 305 when depending directly or indirectly from embodiment 271, wherein the reference sequence is

(SEQ ID NO: 297)
CUUGGGUGGGAGCAGCCACGGGU.

317. The gRNA of any one of embodiments 271 and 272 to 305 when depending directly or indirectly from embodiment 271, wherein the reference sequence is

(SEQ ID NO: 298)
CUUGGGUGGGAGCAGCCGCGGGU.

318. The gRNA of any one of embodiments 271 and 272 to 305 when depending directly or indirectly from embodiment 271, wherein the reference sequence is

(SEQ ID NO: 299)
GAGCAGCCRCGGGUCAGCCACAA.

319. The gRNA of any one of embodiments 271 and 272 to 305 when depending directly or indirectly from embodiment 271, wherein the reference sequence is

(SEQ ID NO: 300)
GAGCAGCCACGGGUCAGCCACAA.

320. The gRNA of any one of embodiments 271 and 272 to 305 when depending directly or indirectly from embodiment 271, wherein the reference sequence is

(SEQ ID NO: 301)
GAGCAGCCGCGGGUCAGCCACAA.

321. The gRNA of any one of embodiments 271 and 272 to 305 when depending directly or indirectly from embodiment 271, wherein the reference sequence is

(SEQ ID NO: 302)
CAUGGCUGUGGCCCUUGUGGCUG.

322. The gRNA of any one of embodiments 271 and 272 to 305 when depending directly or indirectly from embodiment 271, wherein the reference sequence is

(SEQ ID NO: 303)
GUGGGAGCAGCCRCGGGUCAGCC.

323. The gRNA of any one of embodiments 271 and 272 to 305 when depending directly or indirectly from embodiment 271, wherein the reference sequence is

(SEQ ID NO: 304)
GUGGGAGCAGCCACGGGUCAGCC.

324. The gRNA of any one of embodiments 271 and 272 to 305 when depending directly or indirectly from embodiment 271, wherein the reference sequence is

(SEQ ID NO: 305)
GUGGGAGCAGCCGCGGGUCAGCC.

325. The gRNA of any one of embodiments 271 and 272 to 305 when depending directly or indirectly from embodiment 271, wherein the reference sequence is

(SEQ ID NO: 306)
GGCUGACCCGYGGCUGCUCCCAC.

326. The gRNA of any one of embodiments 271 and 272 to 305 when depending directly or indirectly from embodiment 271, wherein the reference sequence is

(SEQ ID NO: 307)
GGCUGACCCGUGGCUGCUCCCAC.

327. The gRNA of any one of embodiments 271 and 272 to 305 when depending directly or indirectly from embodiment 271, wherein the reference sequence is

(SEQ ID NO: 308)
GGCUGACCCGCGGCUGCUCCCAC,

328. The gRNA of any one of embodiments 272 and 273 to 305 when depending directly or indirectly from embodiment 272, wherein the reference sequence is

(SEQ ID NO: 309)
CAGAAAGAGAGAGUAGCGCGAGC.

329. The gRNA of any one of embodiments 272 and 273 to 305 when depending directly or indirectly from embodiment 272, wherein the reference sequence is CACGUCAUCCAGCAGAGAAUGGA (SEQ ID NO:310).

330. The gRNA of any one of embodiments 272 and 273 to 305 when depending directly or indirectly from embodiment 272, wherein the reference sequence is CAUUCUUCAGUAAGUCAACUUCA (SEQ ID NO:311).

331. The gRNA of any one of embodiments 272 and 273 to 305 when depending directly or indirectly from embodiment 272, wherein the reference sequence is

(SEQ ID NO: 312)
AGCAUUCGGGCCGAGAUGUCUCG.

332. The gRNA of any one of embodiments 272 and 273 to 305 when depending directly or indirectly from embodiment 272, wherein the reference sequence is

(SEQ ID NO: 313)
GAGAUGUCUCGCUCCGUGGCCUU.

333. The gRNA of any one of embodiments 272 and 273 to 305 when depending directly or indirectly from embodiment 272, wherein the reference sequence is

(SEQ ID NO: 314)
GGAUAGCCUCCAGGCCAGAAAGA.

334. The gRNA of any one of embodiments 272 and 273 to 305 when depending directly or indirectly from embodiment 272, wherein the reference sequence is

(SEQ ID NO: 315)
UUGACUUUCCAUUCUCUGCUGGA.

335. The gRNA of any one of embodiments 272 and 273 to 305 when depending directly or indirectly from embodiment 272, wherein the reference sequence is GGAAAGUCAAAUUUCCUGAAUUG (SEQ ID NO:316).

336. The gRNA of any one of embodiments 273 and 274 to 305 when depending directly or indirectly from embodiment 273, wherein the reference sequence is UUUGUCUGUGAUAUACACAUCAG (SEQ ID NO:317).

337. The gRNA of any one of embodiments 273 and 274 to 305 when depending directly or indirectly from embodiment 273, wherein the reference sequence is

(SEQ ID NO: 318)
GUAAGGAUUCUGAUGUGUAUAUCA.

338. The gRNA of any one of embodiments 273 and 274 to 305 when depending directly or indirectly from embodiment 273, wherein the reference sequence is

(SEQ ID NO: 319)
GGCCACAGCACUGUUGCUCUUGA.

339. The gRNA of any one of embodiments 273 and 274 to 305 when depending directly or indirectly from embodiment 273, wherein the reference sequence is

(SEQ ID NO: 320)
GAGCAACAGUGCUGUGGCCUGGA.

340. The gRNA of any one of embodiments 273 and 274 to 305 when depending directly or indirectly from embodiment 273, wherein the reference sequence is GGAGCAACAAAUCUGACUUUGCA (SEQ ID NO:321).

341. The gRNA of any one of embodiments 273 and 274 to 305 when depending directly or indirectly from embodiment 273, wherein the reference sequence is AAUGUGUCACAAAGUAAGGAUUC (SEQ ID NO:322).

342. The gRNA of any one of embodiments 273 and 274 to 305 when depending directly or indirectly from embodiment 273, wherein the reference sequence is UCUGAUGUGUAUAUCACAGACAA (SEQ ID NO:323).

343. The gRNA of any one of embodiments 273 and 274 to 305 when depending directly or indirectly from embodiment 273, wherein the reference sequence is

(SEQ ID NO: 324)
GCCUGGAGCAACAAAUCUGACUUU.

344. The gRNA of any one of embodiments 273 and 274 to 305 when depending directly or indirectly from embodiment 273, wherein the reference sequence is

(SEQ ID NO: 325)
GGCGUUUGCACAUGCAAAGUCAG.

345. The gRNA of any one of embodiments 273 and 274 to 305 when depending directly or indirectly from embodiment 273, wherein the reference sequence is

(SEQ ID NO: 326)
GAAGAAGGUGUCUUCUGGAAUAA.

346. The gRNA of any one of embodiments 273 and 274 to 305 when depending directly or indirectly from embodiment 273, wherein the reference sequence is

(SEQ ID NO: 327)
GCUGCCCUUACCUGGGCUGGGGAA.

347. The gRNA of any one of embodiments 274 and 275 to 305 when depending directly or indirectly from embodiment 274, wherein the reference sequence is

(SEQ ID NO: 328)
UGGGCUGGCGGCCAGGAUGGUUC.

348. The gRNA of any one of embodiments 274 and 275 to 305 when depending directly or indirectly from embodiment 274, wherein the reference sequence is

(SEQ ID NO: 329)
CCGCCCAGACGACUGGCCAGGGC.

349. The gRNA of any one of embodiments 274 and 275 to 305 when depending directly or indirectly from embodiment 274, wherein the reference sequence is

(SEQ ID NO: 330)
CAGGCGCCCUGGCCAGUCGUCUG.

350. The gRNA of any one of embodiments 274 and 275 to 305 when depending directly or indirectly from embodiment 274, wherein the reference sequence is

(SEQ ID NO: 331)
AGAACCAUCCUGGCCGCCAGCCC.

351. The gRNA of any one of embodiments 274 and 275 to 305 when depending directly or indirectly from embodiment 274, wherein the reference sequence is

(SEQ ID NO: 332)
CUAAGAACCAUCCUGGCCGCCAG.

352. The gRNA of any one of embodiments 274 and 275 to 305 when depending directly or indirectly from embodiment 274, wherein the reference sequence is

(SEQ ID NO: 333)
GCUGGCGGCCAGGAUGGUUCUUAG.

353. The gRNA of any one of embodiments 274 and 275 to 305 when depending directly or indirectly from embodiment 274, wherein the reference sequence is

(SEQ ID NO: 334)
ACAACUGGGCUGGCGGCCAGGAU.

354. The gRNA of any one of embodiments 274 and 275 to 305 when depending directly or indirectly from embodiment 274, wherein the reference sequence is

(SEQ ID NO: 335)
GUGGGGCUGCUCCAGGCAUGCAG.

355. The gRNA of any one of embodiments 274 and 275 to 305 when depending directly or indirectly from embodiment 274, wherein the reference sequence is

(SEQ ID NO: 336)
GGCCGCCAGCCCAGUUGUAGCAC.

356. The gRNA of any one of embodiments 256 to 355, which is a single guide RNA (sgRNA).

357. A gRNA comprising a spacer and a sgRNA scaffold, which is optionally a gRNA according to any one of embodiments 256 to 356, wherein:
(a) the spacer is positioned 5′ to the sgRNA scaffold; and
(b) the nucleotide sequence of the sgRNA scaffold comprises a nucleotide sequence that is at least 50% identical to a reference scaffold sequence, wherein the reference scaffold sequence is any one of SEQ ID NOS:77-92.

358. A gRNA comprising a means for binding a target mammalian genomic sequence and a sgRNA scaffold, optionally wherein the means for binding a target mammalian genomic sequence is a spacer, wherein:
(a) the means for binding a target genomic sequence is positioned 5′ to the sgRNA scaffold; and
(b) the nucleotide sequence of the sgRNA scaffold comprises a nucleotide sequence that is at least 50% identical to a reference scaffold sequence, wherein the reference scaffold sequence is any one of SEQ ID NOS:77-92.

359. The gRNA of embodiment 357 or embodiment 358, wherein the sgRNA scaffold comprises one or more G:C couples not present in the reference scaffold sequence.

360. The gRNA of any one of embodiments 357 to 358, wherein the sgRNA scaffold comprises one or more U to A substitutions relative to the reference scaffold sequence.

361. The gRNA of any one of embodiments 357 to 360, wherein the sgRNA scaffold comprises one or more trimmed stem loop sequences in place of one or more longer stem loop sequences in the reference scaffold sequence.

362. The gRNA of embodiment 361, wherein the trimmed stem loop sequence comprises a GAAA tetraloop in place of a longer stem loop sequence in the reference scaffold sequence.

363. The gRNA of any one of embodiments 357 to 362, wherein the sgRNA scaffold comprises one or more trimmed loop sequences in place of one or more longer loop sequences in the reference scaffold sequence.

364. The gRNA of embodiment 363, wherein the sgRNA scaffold comprises a GAAA tetraloop in place of a longer loop sequence in the reference scaffold sequence.

365. The gRNA of any one of embodiments 357 to 364, wherein the sgRNA scaffold comprises a nucleotide sequence that is at least 55% identical to the reference scaffold sequence.

366. The gRNA of embodiment 365, wherein the sgRNA scaffold comprises a nucleotide sequence that is at least 60% identical to the reference scaffold sequence.

367. The gRNA of embodiment 365, wherein the sgRNA scaffold comprises a nucleotide sequence that is at least 65% identical to the reference scaffold sequence.

368. The gRNA of embodiment 365, wherein the sgRNA scaffold comprises a nucleotide sequence that is at least 70% identical to the reference scaffold sequence.

369. The gRNA of embodiment 365, wherein the sgRNA scaffold comprises a nucleotide sequence that is at least 75% identical to the reference scaffold sequence.

370. The gRNA of embodiment 365, wherein the sgRNA scaffold comprises a nucleotide sequence that is at least 80% identical to the reference scaffold sequence.

371. The gRNA of embodiment 365, wherein the sgRNA scaffold comprises a nucleotide sequence that is at least 85% identical to the reference scaffold sequence.

372. The gRNA of embodiment 365, wherein the sgRNA scaffold comprises a nucleotide sequence that is at least 90% identical to the reference scaffold sequence.

373. The gRNA of embodiment 365, wherein the sgRNA scaffold comprises a nucleotide sequence that is at least 95% identical to the reference scaffold sequence.

374. The gRNA of embodiment 365, wherein the sgRNA scaffold comprises a nucleotide sequence that is at least 96% identical to the reference scaffold sequence.

375. The gRNA of embodiment 365, wherein the sgRNA scaffold comprises a nucleotide sequence that is at least 97% identical to the reference scaffold sequence.

376. The gRNA of embodiment 365, wherein the sgRNA scaffold comprises a nucleotide sequence that is at least 98% identical to the reference scaffold sequence.

377. The gRNA of embodiment 365, wherein the sgRNA scaffold comprises a nucleotide sequence that is at least 99% identical to the reference scaffold sequence.

378. The gRNA of embodiment 365, wherein the sgRNA scaffold comprises a nucleotide sequence that has no more than 5 nucleotide mismatches with the reference scaffold sequence.

379. The gRNA of embodiment 365, wherein the sgRNA scaffold comprises a nucleotide sequence that has no more than 4 nucleotide mismatches with the reference scaffold sequence.

380. The gRNA of embodiment 365, wherein the sgRNA scaffold comprises a nucleotide sequence that has no more than 3 nucleotide mismatches with the reference scaffold sequence.

381. The gRNA of embodiment 365, wherein the sgRNA scaffold comprises a nucleotide sequence that has no more than 2 nucleotide mismatches with the reference scaffold sequence.

382. The gRNA of embodiment 365, wherein the sgRNA scaffold comprises a nucleotide sequence that has no more than 1 nucleotide mismatches with the reference scaffold sequence.

383. The gRNA of embodiment 357 or embodiment 358, wherein the sgRNA scaffold comprises a nucleotide sequence that is 100% identical to the reference scaffold sequence.

384. The gRNA of any one of embodiments 357 to 383, wherein the reference scaffold sequence is SEQ ID NO:77 or SEQ ID NO:78.

385. The gRNA of embodiment 384, wherein the reference scaffold sequence is SEQ ID NO:77.

386. The gRNA of embodiment 384, wherein the reference scaffold sequence is SEQ ID NO:78.

387. The gRNA of any one of embodiments 357 to 383, wherein the reference scaffold sequence is SEQ ID NO:79 or SEQ ID NO:80.

388. The gRNA of embodiment 387, wherein the reference scaffold sequence is SEQ ID NO:79.

389. The gRNA of embodiment 387, wherein the reference scaffold sequence is SEQ ID NO:80.

390. The gRNA of any one of embodiments 357 to 383, wherein the reference scaffold sequence is SEQ ID NO:81 or SEQ ID NO:82.

391. The gRNA of embodiment 390, wherein the reference scaffold sequence is SEQ ID NO:81.

392. The gRNA of embodiment 390, wherein the reference scaffold sequence is SEQ ID NO:82.

393. The gRNA of any one of embodiments 357 to 383, wherein the reference scaffold sequence is SEQ ID NO:83 or SEQ ID NO:84.

394. The gRNA of embodiment 393, wherein the reference scaffold sequence is SEQ ID NO:83.

395. The gRNA of embodiment 393, wherein the reference scaffold sequence is SEQ ID NO:84.

396. The gRNA of any one of embodiments 357 to 383, wherein the reference scaffold sequence is SEQ ID NO:85 or SEQ ID NO:86.

397. The gRNA of embodiment 396, wherein the reference scaffold sequence is SEQ ID NO:85.

398. The gRNA of embodiment 396, wherein the reference scaffold sequence is SEQ ID NO:86.

399. The gRNA of any one of embodiments 357 to 383, wherein the reference scaffold sequence is SEQ ID NO:87 or SEQ ID NO:88.

400. The gRNA of embodiment 399, wherein the reference scaffold sequence is SEQ ID NO:87.

401. The gRNA of embodiment 399, wherein the reference scaffold sequence is SEQ ID NO:88.

402. The gRNA of any one of embodiments 357 to 383, wherein the reference scaffold sequence is SEQ ID NO:89 or SEQ ID NO:90.

403. The gRNA of embodiment 402, wherein the reference scaffold sequence is SEQ ID NO:89.

404. The gRNA of embodiment 402, wherein the reference scaffold sequence is SEQ ID NO:90.

405. The gRNA of any one of embodiments 357 to 383, wherein the reference scaffold sequence is SEQ ID NO:91 or SEQ ID NO:92.

406. The gRNA of embodiment 405, wherein the reference scaffold sequence is SEQ ID NO:91.

407. The gRNA of embodiment 405, wherein the reference scaffold sequence is SEQ ID NO:92.

408. The gRNA of embodiment 357 or embodiment 358, wherein the nucleotide sequence of the sgRNA scaffold comprises the nucleotide sequence of SEQ ID NO:77.

409. The gRNA of embodiment 357 or embodiment 358, wherein the nucleotide sequence of the sgRNA scaffold comprises the nucleotide sequence of SEQ ID NO:78.

410. The gRNA of embodiment 357 or embodiment 358, wherein the nucleotide sequence of the sgRNA scaffold comprises the nucleotide sequence of SEQ ID NO:79.

411. The gRNA of embodiment 357 or embodiment 358, wherein the nucleotide sequence of the sgRNA scaffold comprises the nucleotide sequence of SEQ ID NO:80.

412. The gRNA of embodiment 357 or embodiment 358, wherein the nucleotide sequence of the sgRNA scaffold comprises the nucleotide sequence of SEQ ID NO:81.

413. The gRNA of embodiment 357 or embodiment 358, wherein the nucleotide sequence of the sgRNA scaffold comprises the nucleotide sequence of SEQ ID NO:82.

414. The gRNA of embodiment 357 or embodiment 358, wherein the nucleotide sequence of the sgRNA scaffold comprises the nucleotide sequence of SEQ ID NO:83.

415. The gRNA of embodiment 357 or embodiment 358, wherein the nucleotide sequence of the sgRNA scaffold comprises the nucleotide sequence of SEQ ID NO:84.

416. The gRNA of embodiment 357 or embodiment 358, wherein the nucleotide sequence of the sgRNA scaffold comprises the nucleotide sequence of SEQ ID NO:85.

417. The gRNA of embodiment 357 or embodiment 358, wherein the nucleotide sequence of the sgRNA scaffold comprises the nucleotide sequence of SEQ ID NO:86.

418. The gRNA of embodiment 357 or embodiment 358, wherein the nucleotide sequence of the sgRNA scaffold comprises the nucleotide sequence of SEQ ID NO:87.

419. The gRNA of embodiment 357 or embodiment 358, wherein the nucleotide sequence of the sgRNA scaffold comprises the nucleotide sequence of SEQ ID NO:88.

420. The gRNA of embodiment 357 or embodiment 358, wherein the nucleotide sequence of the sgRNA scaffold comprises the nucleotide sequence of SEQ ID NO:89.

421. The gRNA of embodiment 357 or embodiment 358, wherein the nucleotide sequence of the sgRNA scaffold comprises the nucleotide sequence of SEQ ID NO:90.

422. The gRNA of embodiment 357 or embodiment 358, wherein the nucleotide sequence of the sgRNA scaffold comprises the nucleotide sequence of SEQ ID NO:91.

423. The gRNA of embodiment 357 or embodiment 358, wherein the nucleotide sequence of the sgRNA scaffold comprises the nucleotide sequence of SEQ ID NO:92.

424. The gRNA of any one of embodiments 357 to 423, wherein the sgRNA scaffold comprises 1 to 8 uracils at its 3′ end.

425. The gRNA of embodiment 424, wherein the sgRNA scaffold comprises 1 uracil at its 3′ end.

426. The gRNA of embodiment 424, wherein the sgRNA scaffold comprises 2 uracils at its 3′ end.

427. The gRNA of embodiment 424, wherein the sgRNA scaffold comprises 3 uracils at its 3′ end.

428. The gRNA of embodiment 424, wherein the sgRNA scaffold comprises 4 uracils at its 3′ end.

429. The gRNA of embodiment 424, wherein the sgRNA scaffold comprises 5 uracils at its 3′ end.

430. The gRNA of embodiment 424, wherein the sgRNA scaffold comprises 6 uracils at its 3′ end.

431. The gRNA of embodiment 424, wherein the sgRNA scaffold comprises 7 uracils at its 3′ end.

432. The gRNA of embodiment 424, wherein the sgRNA scaffold comprises 8 uracils at its 3′ end.

433. The gRNA of embodiment 357 or embodiment 358, wherein the nucleotide sequence of the sgRNA scaffold comprises the nucleotide sequence of any one of SEQ ID NOS:93-108.

434. The gRNA of embodiment 433, wherein the nucleotide sequence of the sgRNA scaffold comprises the nucleotide sequence of SEQ ID NO:93.

435. The gRNA of embodiment 433, wherein the nucleotide sequence of the sgRNA scaffold comprises the nucleotide sequence of SEQ ID NO:94.

436. The gRNA of embodiment 433, wherein the nucleotide sequence of the sgRNA scaffold comprises the nucleotide sequence of SEQ ID NO:95.

437. The gRNA of embodiment 433, wherein the nucleotide sequence of the sgRNA scaffold comprises the nucleotide sequence of SEQ ID NO:96.

438. The gRNA of embodiment 433, wherein the nucleotide sequence of the sgRNA scaffold comprises the nucleotide sequence of SEQ ID NO:97.

439. The gRNA of embodiment 433, wherein the nucleotide sequence of the sgRNA scaffold comprises the nucleotide sequence of SEQ ID NO:98.

440. The gRNA of embodiment 433, wherein the nucleotide sequence of the sgRNA scaffold comprises the nucleotide sequence of SEQ ID NO:99.

441. The gRNA of embodiment 433, wherein the nucleotide sequence of the sgRNA scaffold comprises the nucleotide sequence of SEQ ID NO:100.

442. The gRNA of embodiment 433, wherein the nucleotide sequence of the sgRNA scaffold comprises the nucleotide sequence of SEQ ID NO:101.

443. The gRNA of embodiment 433, wherein the nucleotide sequence of the sgRNA scaffold comprises the nucleotide sequence of SEQ ID NO:102.

444. The gRNA of embodiment 433, wherein the nucleotide sequence of the sgRNA scaffold comprises the nucleotide sequence of SEQ ID NO:103.

445. The gRNA of embodiment 433, wherein the nucleotide sequence of the sgRNA scaffold comprises the nucleotide sequence of SEQ ID NO:104.

446. The gRNA of embodiment 433, wherein the nucleotide sequence of the sgRNA scaffold comprises the nucleotide sequence of SEQ ID NO:105.

447. The gRNA of embodiment 433, wherein the nucleotide sequence of the sgRNA scaffold comprises the nucleotide sequence of SEQ ID NO:106.

448. The gRNA of embodiment 433, wherein the nucleotide sequence of the sgRNA scaffold comprises the nucleotide sequence of SEQ ID NO:107.

449. The gRNA of embodiment 433, wherein the nucleotide sequence of the sgRNA scaffold comprises the nucleotide sequence of SEQ ID NO:108.

450. The gRNA of any one of embodiments 357 to 449, wherein the nucleotide sequence of the spacer is partially or fully complementary to a target mammalian genomic sequence.

451. A gRNA comprising (i) a crRNA comprising a spacer (optionally wherein the spacer is a spacer described in any one of embodiments 271 to 355) and a crRNA scaffold, wherein the spacer is 5′ to the crRNA scaffold, and (ii) a tracrRNA, wherein the nucleotide sequence of the spacer is partially or fully complementary to a target mammalian genomic sequence and the nucleotide sequence of the crRNA scaffold comprises the nucleotide sequence of SEQ ID NO:61, SEQ ID NO:63, SEQ ID NO:65, SEQ ID NO:67, SEQ ID NO:69, SEQ ID NO:71, SEQ ID NO:73, or SEQ ID NO:75.

452. A gRNA comprising (i) a crRNA comprising a means for binding a target mammalian genomic sequence (which is optionally a spacer) and a crRNA scaffold, wherein the means for binding a target mammalian genomic sequence is 5′ to the crRNA scaffold, and (ii) a tracrRNA, wherein the nucleotide sequence of the crRNA scaffold comprises the nucleotide sequence of SEQ ID NO:61, SEQ ID NO:63, SEQ ID NO:65, SEQ ID NO:67, SEQ ID NO:69, SEQ ID NO:71, SEQ ID NO:73, or SEQ ID NO:75.

453. The gRNA of embodiment 451 or 452, wherein the nucleotide sequence of the crRNA scaffold comprises the nucleotide sequence of SEQ ID NO:61.

454. The gRNA of any one of embodiments 451 to 453, wherein the nucleotide sequence of the tracrRNA comprises the nucleotide sequence of SEQ ID NO:62.

455. The gRNA of embodiment 451 or 452, wherein the nucleotide sequence of the crRNA scaffold comprises the nucleotide sequence of SEQ ID NO:63.

456. The gRNA of embodiment 451, embodiment 452, or embodiment 455, wherein the nucleotide sequence of the tracrRNA comprises the nucleotide sequence of SEQ ID NO:64.

457. The gRNA of embodiment 451 or 452, wherein the nucleotide sequence of the crRNA scaffold comprises the nucleotide sequence of SEQ ID NO:65.

458. The gRNA of embodiment 451, embodiment 452, or embodiment 457, wherein the nucleotide sequence of the tracrRNA comprises the nucleotide sequence of SEQ ID NO:66.

459. The gRNA of embodiment 451 or 452, wherein the nucleotide sequence of the crRNA scaffold comprises the nucleotide sequence of SEQ ID NO:67.

460. The gRNA of embodiment 451, embodiment 452, or embodiment 459, wherein the nucleotide sequence of the tracrRNA comprises the nucleotide sequence of SEQ ID NO:68.

461. The gRNA of embodiment 451 or 452, wherein the nucleotide sequence of the crRNA scaffold comprises the nucleotide sequence of SEQ ID NO:69.

462. The gRNA of embodiment 451, embodiment 452, or embodiment 461, wherein the nucleotide sequence of the tracrRNA comprises the nucleotide sequence of SEQ ID NO:70.

463. The gRNA of embodiment 451 or 452, wherein the nucleotide sequence of the crRNA scaffold comprises the nucleotide sequence of SEQ ID NO:71.

464. The gRNA of embodiment 451, embodiment 452, or embodiment 463, wherein the nucleotide sequence of the tracrRNA comprises the nucleotide sequence of SEQ ID NO:72.

465. The gRNA of embodiment 451 or 452, wherein the nucleotide sequence of the crRNA scaffold comprises the nucleotide sequence of SEQ ID NO:73.

466. The gRNA of embodiment 451, embodiment 452, or embodiment 465, wherein the nucleotide sequence of the tracrRNA comprises the nucleotide sequence of SEQ ID NO:74.

467. The gRNA of embodiment 451 or 452, wherein the nucleotide sequence of the crRNA scaffold comprises the nucleotide sequence of SEQ ID NO:75.

468. The gRNA of embodiment 451, embodiment 452, or embodiment 467, wherein the nucleotide sequence of the tracrRNA comprises the nucleotide sequence of SEQ ID NO:76.

469. The gRNA of any one of embodiments 451 to 468, wherein the gRNA comprises separate crRNA and tracrRNA molecules.

470. The gRNA of any one of embodiments 451 to 468, wherein the gRNA is a single guide RNA (sgRNA).

471. The gRNA of any one of embodiments 450 to 470, wherein the target mammalian genomic sequence is a human genomic sequence.

472. The gRNA of embodiment 471, wherein the target mammalian genomic sequence is a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence.

473. The gRNA of embodiment 472, wherein the target mammalian genomic sequence is a CCR5, EMX1, Fas, FANCF, HBB, ZSCAN, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, or BCR genomic sequence.

474. The gRNA of embodiment 471, wherein the target mammalian genomic sequence is a RHO genomic sequence.

475. The gRNA of embodiment 471, wherein the target mammalian genomic sequence is a B2M genomic sequence.

476. The gRNA of embodiment 471, wherein the target mammalian genomic sequence is a TRAC genomic sequence.

477. The gRNA of embodiment 471, wherein the target mammalian genomic sequence is a LAG3 genomic sequence.

478. The gRNA of embodiment 471, wherein the target mammalian genomic sequence is a PD1 genomic sequence.

479. The gRNA of any one of embodiments 450 to 478, wherein the target mammalian genomic sequence is upstream of a protospacer adjacent motif (PAM) sequence in the non-target strand recognized by a Type II Cas protein, optionally wherein the Type II Cas protein is a Type II Cas protein according to any one of embodiments 1 to 255.

480. The gRNA of embodiment 479, wherein the PAM sequence is NNAGG.

481. The gRNA of embodiment 479, wherein the PAM sequence is NNAA.

482. The gRNA of embodiment 479, wherein the PAM sequence is NNAGG.

483. The gRNA of embodiment 479, wherein the PAM sequence is NNG.

484. The gRNA of embodiment 479, wherein the PAM sequence is NNGA.

485. The gRNA of embodiment 479, wherein the PAM sequence is NNAAA.

486. The gRNA of embodiment 479, wherein the PAM sequence is NNGGNNAA.

487. The gRNA of embodiment 479, wherein the PAM sequence is NYRRR.

488. The gRNA of embodiment 479, wherein the PAM sequence is NNRRR.

489. The gRNA of embodiment 479, wherein the PAM sequence is NYARR.

490. The gRNA of embodiment 479, wherein the PAM sequence is NYAAR.

491. The gRNA of embodiment 479, wherein the PAM sequence is NYARRNY.

492. The gRNA of embodiment 479, wherein the PAM sequence is NYAARNY.

493. The gRNA of embodiment 479, wherein the PAM sequence is NYRRR.

494. The gRNA of embodiment 479, wherein the PAM sequence is NNARR.

495. The gRNA of embodiment 479, wherein the PAM sequence is NYAAR.

496. The gRNA of embodiment 479, wherein the PAM sequence is NYARR.

497. The gRNA of embodiment 479, wherein the PAM sequence is NNRGR.

498. The gRNA of embodiment 479, wherein the PAM sequence is NYRGR.

499. The gRNA of embodiment 479, wherein the PAM sequence is NYAGG.

500. The gRNA of embodiment 479, wherein the PAM sequence is NYRGG.

501. The gRNA of embodiment 479, wherein the PAM sequence is NNAGG.

502. The gRNA of embodiment 479, wherein the PAM sequence is NNGNNNYN.

503. The gRNA of embodiment 479, wherein the PAM sequence is NNGNNNYR.

504. The gRNA of embodiment 479, wherein the PAM sequence is NNGYNNYR.

505. The gRNA of embodiment 479, wherein the PAM sequence is NNGYNNCR.

506. The gRNA of embodiment 479, wherein the PAM sequence is NNGNVHYR.

507. The gRNA of embodiment 479, wherein the PAM sequence is NNGYVHYR.

508. The gRNA of embodiment 479, wherein the PAM sequence is NNGYVHCR.

509. The gRNA of embodiment 479, wherein the PAM sequence is NNGHNHYR.

510. The gRNA of embodiment 479, wherein the PAM sequence is NNGRR.

511. The gRNA of embodiment 479, wherein the PAM sequence is NYGRR.

512. The gRNA of embodiment 479, wherein the PAM sequence is NYRRR.

513. The gRNA of embodiment 479, wherein the PAM sequence is NNARR.

514. The gRNA of embodiment 479, wherein the PAM sequence is NYRRRNY.

515. The gRNA of embodiment 479, wherein the PAM sequence is NYARGNY.

516. The gRNA of embodiment 479, wherein the PAM sequence is NYARRNY.

517. The gRNA of embodiment 479, wherein the PAM sequence is NNGG.

518. The gRNA of embodiment 479, wherein the PAM sequence is NNGGWW.

519. The gRNA of embodiment 479, wherein the PAM sequence is NNGGAW.

520. The gRNA of embodiment 479, wherein the PAM sequence is NNNNCNNA.

521. The gRNA of embodiment 479, wherein the PAM sequence is NNNNCKNA.

522. The gRNA of embodiment 479, wherein the PAM sequence is NNNNGT.

523. The gRNA of embodiment 479, wherein the PAM sequence is NNNNCNNA.

524. The gRNA of embodiment 479, wherein the PAM sequence is NNNNCC.

525. The gRNA of embodiment 479, wherein the PAM sequence is NNNNCMNA.

526. The gRNA of embodiment 479, wherein the PAM sequence is NNNNCCNA.

527. The gRNA of any one of embodiments 357 to 526, wherein the spacer is 15 to 30 nucleotides in length.

528. The gRNA of embodiment 527, wherein the spacer is 15 to 25 nucleotides in length.

529. The gRNA of embodiment 527, wherein the spacer is 16 to 24 nucleotides in length.

530. The gRNA of embodiment 527, wherein the spacer is 17 to 23 nucleotides in length.

531. The gRNA of embodiment 527, wherein the spacer is 18 to 22 nucleotides in length.

532. The gRNA of embodiment 527, wherein the spacer is 19 to 21 nucleotides in length.

533. The gRNA of embodiment 527, wherein the spacer is 18 to 30 nucleotides in length.

534. The gRNA of embodiment 527, wherein the spacer is 20 to 28 nucleotides in length.

535. The gRNA of embodiment 527, wherein the spacer is 22 to 26 nucleotides in length.

536. The gRNA of embodiment 527, wherein the spacer is 23 to 25 nucleotides in length.

537. The gRNA of embodiment 527, wherein the spacer is 20 nucleotides in length.

538. The gRNA of embodiment 527, wherein the spacer is 21 nucleotides in length.

539. The gRNA of embodiment 527, wherein the spacer is 22 nucleotides in length.

540. The gRNA of embodiment 527, wherein the spacer is 23 nucleotides in length.

541. The gRNA of embodiment 527, wherein the spacer is 24 nucleotides in length.

542. The gRNA of embodiment 527, wherein the spacer is 25 nucleotides in length.

543. The gRNA of embodiment 527, wherein the spacer is 26 nucleotides in length.

544. The gRNA of embodiment 527, wherein the spacer is 27 nucleotides in length.

545. The gRNA of embodiment 527, wherein the spacer is 28 nucleotides in length.

546. A gRNA comprising a spacer comprising the sequence of SEQ ID NO:287.

547. A gRNA comprising a spacer comprising the sequence of SEQ ID NO:288.

548. A gRNA comprising a spacer comprising the sequence of SEQ ID NO:289.

549. A gRNA comprising a spacer comprising the sequence of SEQ ID NO:290.

550. A gRNA comprising a spacer comprising the sequence of SEQ ID NO:291.

551. A gRNA comprising a spacer comprising the sequence of SEQ ID NO:292.

552. A gRNA comprising a spacer comprising the sequence of SEQ ID NO:293.

553. A gRNA comprising a spacer comprising the sequence of SEQ ID NO:294.

554. A gRNA comprising a spacer comprising the sequence of SEQ ID NO:295.

555. A gRNA comprising a spacer comprising the sequence of SEQ ID NO:296.

556. A gRNA comprising a spacer comprising the sequence of SEQ ID NO:297.

557. A gRNA comprising a spacer comprising the sequence of SEQ ID NO:298.

558. A gRNA comprising a spacer comprising the sequence of SEQ ID NO:299.

559. A gRNA comprising a spacer comprising the sequence of SEQ ID NO:300.

560. A gRNA comprising a spacer comprising the sequence of SEQ ID NO:301.

561. A gRNA comprising a spacer comprising the sequence of SEQ ID NO:302.

562. A gRNA comprising a spacer comprising the sequence of SEQ ID NO:303.

563. A gRNA comprising a spacer comprising the sequence of SEQ ID NO:304.

564. A gRNA comprising a spacer comprising the sequence of SEQ ID NO:305.

565. A gRNA comprising a spacer comprising the sequence of SEQ ID NO:306.

566. A gRNA comprising a spacer comprising the sequence of SEQ ID NO:307.

567. A gRNA comprising a spacer comprising the sequence of SEQ ID NO:308.

568. A gRNA comprising a spacer comprising the sequence of SEQ ID NO:309.

569. A gRNA comprising a spacer comprising the sequence of SEQ ID NO:310.

570. A gRNA comprising a spacer comprising the sequence of SEQ ID NO:311.

571. A gRNA comprising a spacer comprising the sequence of SEQ ID NO:312.

572. A gRNA comprising a spacer comprising the sequence of SEQ ID NO:313.

573. A gRNA comprising a spacer comprising the sequence of SEQ ID NO:314.

574. A gRNA comprising a spacer comprising the sequence of SEQ ID NO:315.

575. A gRNA comprising a spacer comprising the sequence of SEQ ID NO:316.

576. A gRNA comprising a spacer comprising the sequence of SEQ ID NO:317.

577. A gRNA comprising a spacer comprising the sequence of SEQ ID NO:318.

578. A gRNA comprising a spacer comprising the sequence of SEQ ID NO:319.

579. A gRNA comprising a spacer comprising the sequence of SEQ ID NO:320.

580. A gRNA comprising a spacer comprising the sequence of SEQ ID NO:321.

581. A gRNA comprising a spacer comprising the sequence of SEQ ID NO:322.

582. A gRNA comprising a spacer comprising the sequence of SEQ ID NO:323.

583. A gRNA comprising a spacer comprising the sequence of SEQ ID NO:324.

584. A gRNA comprising a spacer comprising the sequence of SEQ ID NO:325.

585. A gRNA comprising a spacer comprising the sequence of SEQ ID NO:326.

586. A gRNA comprising a spacer comprising the sequence of SEQ ID NO:327.

587. A gRNA comprising a spacer comprising the sequence of SEQ ID NO:328.

588. A gRNA comprising a spacer comprising the sequence of SEQ ID NO:329.

589. A gRNA comprising a spacer comprising the sequence of SEQ ID NO:330.

590. A gRNA comprising a spacer comprising the sequence of SEQ ID NO:331.

591. A gRNA comprising a spacer comprising the sequence of SEQ ID NO:332.

592. A gRNA comprising a spacer comprising the sequence of SEQ ID NO:333.

593. A gRNA comprising a spacer comprising the sequence of SEQ ID NO:334.

594. A gRNA comprising a spacer comprising the sequence of SEQ ID NO:335.

595. A gRNA comprising a spacer comprising the sequence of SEQ ID NO:336.

596. The gRNA of any one of embodiments 546 to 595, wherein the spacer is positioned 5′ to a gRNA scaffold.

597. The gRNA of any one of embodiments 546 to 560, 568 to 570, 576 to 580, and 593 to 595, wherein the spacer is positioned 5′ to a scaffold whose sequence comprises the sequence of SEQID NO:87.

598. The gRNA of any one of embodiments 546 to 560, 568 to 570, 576 to 580, and 593 to 595, wherein the spacer is positioned 5′ to a scaffold whose sequence comprises the sequence of SEQID NO:88.

599. The gRNA of any one of embodiments 561, 571 to 575, and 581 to 592, wherein the spacer is positioned 5′ to a scaffold whose sequence comprises the sequence of SEQ ID NO:89.

600. The gRNA of any one of embodiments 561, 571 to 575, and 581 to 592, wherein the spacer is positioned 5′ to a scaffold whose sequence comprises the sequence of SEQ ID NO:90.

601. The gRNA of any one of embodiments 562 to 568, wherein the spacer is positioned 5′ to a scaffold whose sequence comprises the sequence of SEQ ID NO:77.

602. The gRNA of any one of embodiments 562 to 568, wherein the spacer is positioned 5′ to a scaffold whose sequence comprises the sequence of SEQ ID NO:78.

603. The gRNA of any one of embodiments 596 to 602, which comprises 1 to 8 uracils at the 3′ end.

604. The gRNA of any one of embodiments 596 to 602, which comprises 1 uracil at the 3′ end.

605. The gRNA of any one of embodiments 596 to 602, which comprises 2 uracils at the 3′ end.

606. The gRNA of any one of embodiments 596 to 602, which comprises 3 uracils at the 3′ end.

607. The gRNA of any one of embodiments 596 to 602, which comprises 4 uracils at the 3′ end.

608. The gRNA of any one of embodiments 596 to 602, which comprises 5 uracils at the 3′ end.

609. The gRNA of any one of embodiments 596 to 602, which comprises 6 uracils at the 3′ end.

610. The gRNA of any one of embodiments 596 to 602, which comprises 7 uracils at the 3′ end.

611. The gRNA of any one of embodiments 596 to 602, which comprises 8 uracils at the 3′ end.

612. A combination of gRNAs comprising a first gRNA and a second gRNA independently selected from gRNAs of embodiments 256 to 611.

613. A combination of gRNAs comprising a first gRNA selected from gRNAs of embodiments 256 to 611 provided that the first gRNA targets RHO and a second gRNA targeting RHO intron 1.

614. A system comprising the Type II Cas protein of any one of embodiments 1 to 255 and a guide RNA (gRNA) comprising a spacer sequence, optionally wherein the gRNA is a gRNA according to any one of embodiments 256 to 611.

615. A system comprising the Type II Cas protein of any one of embodiments 1 to 255 and a means for targeting the Type II Cas protein to a target genomic sequence, optionally wherein the means for targeting the Type II Cas protein to a target genomic sequence is a guide RNA (gRNA) molecule, optionally as described in in any one of embodiments 256 to 611, optionally wherein the gRNA molecule comprises a spacer partially or fully complementary to a target mammalian genomic sequence.

616. The system of embodiment 614, wherein the spacer sequence is partially or fully complementary to a target mammalian genomic sequence.

617. The system of any one of embodiments 615 to 616, wherein the target mammalian genomic sequence is a human genomic sequence.

618. The system of embodiment 617, wherein the target mammalian genomic sequence is a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence.

619. The system of embodiment 617, wherein the target mammalian genomic sequence is a RHO genomic sequence.

620. The system of embodiment 617, wherein the target mammalian genomic sequence is a RHO genomic sequence is a B2M genomic sequence.

621. The system of embodiment 617, wherein the target mammalian genomic sequence is a RHO genomic sequence is a TRAC genomic sequence.

622. The system of embodiment 617, wherein the target mammalian genomic sequence is a RHO genomic sequence is a PD1 genomic sequence.

623. The system of embodiment 617, wherein the target mammalian genomic sequence is a RHO genomic sequence is a LAG3 genomic sequence.

624. The system of any one of embodiments 615 to 623, wherein the target mammalian genomic sequence is upstream of a protospacer adjacent motif (PAM) sequence in the non-target strand recognized by the Type II Cas protein.

625. The system of embodiment 624, wherein the reference protein sequence is SEQ ID NO:1 or SEQ ID NO:2 and wherein the PAM sequence is NNAGG.

626. The system of embodiment 624, wherein the reference protein sequence is SEQ ID NO:7 or SEQ ID NO:8 and the PAM sequence is NNAA.

627. The system of embodiment 624, wherein the reference protein sequence is SEQ ID NO:13 or SEQ ID NO:14 and the PAM sequence is NNAGG.

628. The system of embodiment 624, wherein the reference protein sequence is SEQ ID NO:19 or SEQ ID NO:20 and the PAM sequence is NNG.

629. The system of embodiment 624, wherein the reference protein sequence is SEQ ID NO:25 or SEQ ID NO:26 and the PAM sequence is NNGA.

630. The system of embodiment 624, wherein the reference protein sequence is SEQ ID NO:31 or SEQ ID NO:32 and the PAM sequence is NNAAA.

631. The system of embodiment 624, wherein the reference protein sequence is SEQ ID NO:37 or SEQ ID NO:38 and the PAM sequence is NNGGNNAA.

632. The system of embodiment 624, wherein the reference protein sequence is SEQ ID NO:1 or SEQ ID NO:2 and wherein the PAM sequence is NYRRR.

633. The system of embodiment 624, wherein the reference protein sequence is SEQ ID NO:1 or SEQ ID NO:2 and wherein the PAM sequence is NNRRR.

634. The system of embodiment 624, wherein the reference protein sequence is SEQ ID NO:1 or SEQ ID NO:2 and wherein the PAM sequence is NYARR.

635. The system of embodiment 624, wherein the reference protein sequence is SEQ ID NO:1 or SEQ ID NO:2 and wherein the PAM sequence is NYAAR.

636. The system of embodiment 624, wherein the reference protein sequence is SEQ ID NO:1 or SEQ ID NO:2 and wherein the PAM sequence is NYARRNY.

637. The system of embodiment 624, wherein the reference protein sequence is SEQ ID NO:1 or SEQ ID NO:2 and wherein the PAM sequence is NYAARNY.

638. The system of embodiment 624, wherein the reference protein sequence is SEQ ID NO:7 or SEQ ID NO:8 and wherein the PAM sequence is NYRRR.

639. The system of embodiment 624, wherein the reference protein sequence is SEQ ID NO:7 or SEQ ID NO:8 and wherein the PAM sequence is NNARR.

640. The system of embodiment 624, wherein the reference protein sequence is SEQ ID NO:7 or SEQ ID NO:8 and wherein the PAM sequence is NYAAR.

641. The system of embodiment 624, wherein the reference protein sequence is SEQ ID NO:7 or SEQ ID NO:8 and wherein the PAM sequence is NYARR.

642. The system of embodiment 624, wherein the reference protein sequence is SEQ ID NO:13 or SEQ ID NO:14 and wherein the PAM sequence is NNRGR.

643. The system of embodiment 624, wherein the reference protein sequence is SEQ ID NO:13 or SEQ ID NO:14 and wherein the PAM sequence is NYRGR.

644. The system of embodiment 624, wherein the reference protein sequence is SEQ ID NO:13 or SEQ ID NO:14 and wherein the PAM sequence is NYAGG.

645. The system of embodiment 624, wherein the reference protein sequence is SEQ ID NO:13 or SEQ ID NO:14 and wherein the PAM sequence is NYRGG.

646. The system of embodiment 624, wherein the reference protein sequence is SEQ ID NO:13 or SEQ ID NO:14 and wherein the PAM sequence is NNAGG.

647. The system of embodiment 624, wherein the reference protein sequence is SEQ ID NO:19 or SEQ ID NO:20 and wherein the PAM sequence is NNGNNNYN.

648. The system of embodiment 624, wherein the reference protein sequence is SEQ ID NO:19 or SEQ ID NO:20 and wherein the PAM sequence is NNGNNNYR.

649. The system of embodiment 624, wherein the reference protein sequence is SEQ ID NO:19 or SEQ ID NO:20 and wherein the PAM sequence is NNGYNNYR.

650. The system of embodiment 624, wherein the reference protein sequence is SEQ ID NO:19 or SEQ ID NO:20 and wherein the PAM sequence is NNGYNNCR.

651. The system of embodiment 624, wherein the reference protein sequence is SEQ ID NO:19 or SEQ ID NO:20 and wherein the PAM sequence is NNGNVHYR.

652. The system of embodiment 624, wherein the reference protein sequence is SEQ ID NO:19 or SEQ ID NO:20 and wherein the PAM sequence is NNGYVHYR.

653. The system of embodiment 624, wherein the reference protein sequence is SEQ ID NO:19 or SEQ ID NO:20 and wherein the PAM sequence is NNGYVHCR.

654. The system of embodiment 624, wherein the reference protein sequence is SEQ ID NO:19 or SEQ ID NO:20 and wherein the PAM sequence is NNGHNHYR.

655. The system of embodiment 624, wherein the reference protein sequence is SEQ ID NO:25 or SEQ ID NO:26 and wherein the PAM sequence is NNGRR.

656. The system of embodiment 624, wherein the reference protein sequence is SEQ ID NO:25 or SEQ ID NO:26 and wherein the PAM sequence is NYGRR.

657. The system of embodiment 624, wherein the reference protein sequence is SEQ ID NO:31 or SEQ ID NO:32 and wherein the PAM sequence is NYRRR.

658. The system of embodiment 624, wherein the reference protein sequence is SEQ ID NO:31 or SEQ ID NO:32 and wherein the PAM sequence is NNARR.

659. The system of embodiment 624, wherein the reference protein sequence is SEQ ID NO:31 or SEQ ID NO:32 and wherein the PAM sequence is NYRRRNY.

660. The system of embodiment 624, wherein the reference protein sequence is SEQ ID NO:31 or SEQ ID NO:32 and wherein the PAM sequence is NYARGNY.

661. The system of embodiment 624, wherein the reference protein sequence is SEQ ID NO:31 or SEQ ID NO:32 and wherein the PAM sequence is NYARRNY.

662. The system of embodiment 624, wherein the reference protein sequence is SEQ ID NO:37 or SEQ ID NO:38 and wherein the PAM sequence is NNGG.

663. The system of embodiment 624, wherein the reference protein sequence is SEQ ID NO:37 or SEQ ID NO:38 and wherein the PAM sequence is NNGGWW.

664. The system of embodiment 624, wherein the reference protein sequence is SEQ ID NO:37 or SEQ ID NO:38 and wherein the PAM sequence is NNGGAW.

665. The system of embodiment 624, wherein the reference protein sequence is SEQ ID NO:43 or SEQ ID NO:44 and wherein the PAM sequence is NNNNCNNA.

666. The system of embodiment 624, wherein the reference protein sequence is SEQ ID NO:43 or SEQ ID NO:44 and wherein the PAM sequence is NNNNCKNA.

667. The system of embodiment 624, wherein the reference protein sequence is SEQ ID NO:49 or SEQ ID NO:50 and wherein the PAM sequence is NNNNGT.

668. The system of embodiment 624, wherein the reference protein sequence is SEQ ID NO:55 or SEQ ID NO:56 and wherein the PAM sequence is NNNNCNNA.

669. The system of embodiment 624, wherein the reference protein sequence is SEQ ID NO:55 or SEQ ID NO:56 and wherein the PAM sequence is NNNNCC.

670. The system of embodiment 624, wherein the reference protein sequence is SEQ ID NO:55 or SEQ ID NO:56 and wherein the PAM sequence is NNNNCMNA.

671. The system of embodiment 624, wherein the reference protein sequence is SEQ NO:55 or SEQ ID NO:56 and wherein the PAM sequence is NNNNCCNA.

672. The system of any one of embodiments 614 to 671, wherein the gRNA comprises a crRNA sequence and a tracrRNA sequence.

673. The system of embodiment 672, wherein the reference protein sequence is SEQ ID NO:1 or SEQ ID NO:2 and wherein the crRNA sequence comprises the spacer sequence 5′ to the nucleotide sequence of SEQ ID NO:61.

674. The system of embodiment 672 or embodiment 673, wherein the reference protein sequence is SEQ ID NO:1 or SEQ ID NO:2 and wherein the tracrRNA sequence comprises the nucleotide sequence of SEQ ID NO:62.

675. The system of embodiment 672, wherein the reference protein sequence is SEQ ID NO:7 or SEQ ID NO:8 and wherein the crRNA sequence comprises the spacer sequence 5′ to the nucleotide sequence of SEQ ID NO:63.

676. The system of embodiment 672 or embodiment 675, wherein the reference protein sequence is SEQ ID NO:7 or SEQ ID NO:8 and wherein the tracrRNA sequence comprises the nucleotide sequence of SEQ ID NO:64.

677. The system of embodiment 672, wherein the reference protein sequence is SEQ ID NO:13 or SEQ ID NO:14 and wherein the crRNA sequence comprises the spacer sequence 5′ to the nucleotide sequence of SEQ ID NO:65.

678. The system of embodiment 672 or embodiment 677, wherein the reference protein sequence is SEQ ID NO:13 or SEQ ID NO:14 and wherein the tracrRNA sequence comprises the nucleotide sequence of SEQ ID NO:66.

679. The system of embodiment 672, wherein the reference protein sequence is SEQ ID NO:19 or SEQ ID NO:20 and wherein the crRNA sequence comprises the spacer sequence 5′ to the nucleotide sequence of SEQ ID NO:67.

680. The system of embodiment 672 or embodiment 679, wherein the reference protein sequence is SEQ ID NO:19 or SEQ ID NO:20 and wherein the tracrRNA sequence comprises the nucleotide sequence of SEQ ID NO:68.

681. The system of embodiment 672, wherein the reference protein sequence is SEQ ID NO:25 or SEQ ID NO:26 and wherein the crRNA sequence comprises the spacer sequence 5′ to the nucleotide sequence of SEQ ID NO:69.

682. The system of embodiment 672 or embodiment 681, wherein the reference protein sequence is SEQ ID NO:25 or SEQ ID NO:26 and wherein the tracrRNA sequence comprises the nucleotide sequence of SEQ ID NO:70.

683. The system of embodiment 672, wherein the reference protein sequence is SEQ ID NO:31 or SEQ ID NO:32 and wherein the crRNA sequence comprises the spacer sequence 5′ to the nucleotide sequence of SEQ ID NO:69.

684. The system of embodiment 672 or embodiment 683, wherein the reference protein sequence is SEQ ID NO:31 or SEQ ID NO:32 and wherein the tracrRNA sequence comprises the nucleotide sequence of SEQ ID NO:70.

685. The system of embodiment 672, wherein the reference protein sequence is SEQ ID NO:37 or SEQ ID NO:38 and wherein the crRNA sequence comprises the spacer sequence 5′ to the nucleotide sequence of SEQ ID NO:69.

686. The system of embodiment 672 or embodiment 685, wherein the reference protein sequence is SEQ ID NO:37 or SEQ ID NO:38 and wherein the tracrRNA sequence comprises the nucleotide sequence of SEQ ID NO:70.

687. The system of embodiment 672, wherein the reference protein sequence is SEQ ID NO:43 or SEQ ID NO:44 and wherein the crRNA sequence comprises the spacer sequence 5′ to the nucleotide sequence of SEQ ID NO:71.

688. The system of embodiment 672 or embodiment 687, wherein the reference protein sequence is SEQ ID NO:43 or SEQ ID NO:44 and wherein the tracrRNA sequence comprises the nucleotide sequence of SEQ ID NO:72.

689. The system of embodiment 672, wherein the reference protein sequence is SEQ ID NO:49 or SEQ ID NO:50 and wherein the crRNA sequence comprises the spacer sequence 5′ to the nucleotide sequence of SEQ ID NO:73.

690. The system of embodiment 672 or embodiment 689, wherein the reference protein sequence is SEQ ID NO:49 or SEQ ID NO:50 and wherein the tracrRNA sequence comprises the nucleotide sequence of SEQ ID NO:74.

691. The system of embodiment 672, wherein the reference protein sequence is SEQ ID NO:55 or SEQ ID NO:56 and wherein the crRNA sequence comprises the spacer sequence 5′ to the nucleotide sequence of SEQ ID NO:75.

692. The system of embodiment 672 or embodiment 691, wherein the reference protein sequence is SEQ ID NO:55 or SEQ ID NO:56 and wherein the tracrRNA sequence comprises the nucleotide sequence of SEQ ID NO:76.

693. The system of any one of embodiments 627 to 692, wherein the gRNA comprises separate crRNA and tracrRNA molecules.

694. The system of any one of embodiments 546 to 692, wherein the gRNA is a single guide RNA (sgRNA) comprising the spacer and a sgRNA scaffold, wherein the spacer is positioned 5′ to the sgRNA scaffold.

695. The system of embodiment 694, wherein the sgRNA scaffold is a sgRNA scaffold described in any one of embodiments 256 to 611.

696. The system of embodiment 694 or embodiment 695, wherein the sgRNA scaffold comprises the nucleotide sequence of any one SEQ ID NOS:77-108.

697. The system of any one of embodiments 546 to 696, wherein the spacer is a spacer described in any one of embodiments 256 to 611.

698. The system of any one of embodiments 546 to 697, which is a ribonucleoprotein (RNP) comprising the Type II Cas protein complexed to the gRNA or means for targeting the Type II Cas protein to a target genomic sequence.

699. A nucleic acid encoding the Type II Cas protein of any one of embodiments 1 to 255, optionally wherein the nucleotide sequence encoding the Type II Cas protein is operably linked to a promoter that is heterologous to the Type II Cas protein.

700. The nucleic acid of embodiment 699, wherein the nucleotide sequence encoding the Type II Cas protein is codon optimized for expression in human cells.

701. The nucleic acid of embodiment 700, wherein when the reference protein sequence is SEQ ID NO:1 or SEQ ID NO:2, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:5 or SEQ ID NO:6.

702. The nucleic acid of embodiment 700, wherein when the reference protein sequence is SEQ ID NO:7 or SEQ ID NO:8, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:11 or SEQ ID NO:12.

703. The nucleic acid of embodiment 700, wherein when the reference protein sequence is SEQ ID NO:13 or SEQ ID NO:14, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:17 or SEQ ID NO:18.

704. The nucleic acid of embodiment 700, wherein when the reference protein sequence is SEQ ID NO:19 or SEQ ID NO:20, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:23 or SEQ ID NO:24.

705. The nucleic acid of embodiment 700, wherein when the reference protein sequence is SEQ ID NO:25 or SEQ ID NO:26, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:29 or SEQ ID NO:30.

706. The nucleic acid of embodiment 700, wherein when the reference protein sequence is SEQ ID NO:31 or SEQ ID NO:32, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:35 or SEQ ID NO:36.

707. The nucleic acid of embodiment 700, wherein when the reference protein sequence is SEQ ID NO:37 or SEQ ID NO:38, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:41 or SEQ ID NO:42.

708. The nucleic acid of embodiment 700, wherein when the reference protein sequence is SEQ ID NO:43 or SEQ ID NO:44, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:47 or SEQ ID NO:48.

709. The nucleic acid of embodiment 700, wherein when the reference protein sequence is SEQ ID NO:49 or SEQ ID NO:50, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:53 or SEQ ID NO:54.

710. The nucleic acid of embodiment 700, wherein when the reference protein sequence is SEQ ID NO:55 or SEQ ID NO:56, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:59 or SEQ ID NO:60.

711. The nucleic acid of any one of embodiments embodiment 699 to 710, which is a plasmid.

712. The nucleic acid of any one of embodiments embodiment 699 to 710, which is a viral genome.

713. The nucleic acid of embodiment 712, wherein the viral genome is an adeno-associated virus (AAV) genome.

714. The nucleic acid of embodiment 713, wherein the AAV genome is an AAV2, AAV5, AAV7m8, AAV8, AAV9, AAVrh8r, or AAVrh10 genome.

715. The nucleic acid of embodiment 714, wherein the AAV genome is an AAV2 genome. 716. The nucleic acid of embodiment 714, wherein the AAV genome is an AAV5 genome.

717. The nucleic acid of embodiment 714, wherein the AAV genome is an AAV7m8 genome.

718. The nucleic acid of embodiment 714, wherein the AAV genome is an AAV8 genome.

719. The nucleic acid of embodiment 714, wherein the AAV genome is an AAV9 genome.

720. The nucleic acid of embodiment 714, wherein the AAV genome is an AAVrh8r genome.

721. The nucleic acid of embodiment 714, wherein the AAV genome is an AAVrh10 genome.

722. The nucleic acid of any one of embodiments 699 to 721, further encoding a gRNA, optionally wherein the gRNA is a gRNA according to any one of embodiments 256 to 611.

723. The nucleic acid of any one of embodiments 699 to 721, further encoding a combination of gRNAs, optionally wherein the combination of gRNAs is a combination of gRNAs according to any one of embodiments 612 to 613.

724. A nucleic acid encoding the gRNA of any one of embodiments 256 to 611.

725. A nucleic acid encoding the combination of gRNAs of any one of embodiments 612 to 613.

726. The nucleic acid of embodiment 724 or embodiment 725, which is a plasmid.

727. The nucleic acid of embodiment 724 or embodiment 725, which is a viral genome.

728. The nucleic acid of embodiment 727, wherein the viral genome is an adeno-associated virus (AAV) genome.

729. The nucleic acid of embodiment 728, wherein the AAV genome is a AAV2, AAV5, AAV7m8, AAV8, AAV9, AAVrh8r, or AAVrh10 genome.

730. The nucleic acid of embodiment 729, wherein the AAV genome is an AAV2 genome.

731. The nucleic acid of embodiment 729, wherein the AAV genome is an AAV5 genome.

732. The nucleic acid of embodiment 729, wherein the AAV genome is an AAV7m8 genome.

733. The nucleic acid of embodiment 729, wherein the AAV genome is an AAV8 genome.

734. The nucleic acid of embodiment 729, wherein the AAV genome is an AAV9 genome.

735. The nucleic acid of embodiment 729, wherein the AAV genome is an AAVrh8r genome.

736. The nucleic acid of embodiment 729, wherein the AAV genome is an AAVrh10 genome.

737. The nucleic acid of any one of embodiments 724 to 736, further encoding a Type II Cas protein, optionally wherein the Type II Cas protein is a Type II Cas protein according to any one of embodiments 1 to 255.

738. A nucleic acid encoding the Type II Cas protein and gRNA of the system of any one of embodiments 614 to 698.

739. The nucleic acid of embodiment 738, wherein the nucleotide sequence encoding the Type II Cas protein is codon optimized for expression in human cells.

740. The nucleic acid of embodiment 738 or embodiment 739, which is a plasmid.

741. The nucleic acid of embodiment 738 or embodiment 739, which is a viral genome.

742. The nucleic acid of embodiment 741, wherein the viral genome is an adeno-associated virus (AAV) genome.

743. The nucleic acid of embodiment 742, wherein the AAV genome is a AAV2, AAV5, AAV7m8, AAV8, AAV9, AAVrh8r, or AAVrh10 genome.

744. The nucleic acid of embodiment 743, wherein the AAV genome is an AAV2 genome.

745. The nucleic acid of embodiment 743, wherein the AAV genome is an AAV5 genome.

746. The nucleic acid of embodiment 743, wherein the AAV genome is an AAV7m8 genome.

747. The nucleic acid of embodiment 743, wherein the AAV genome is an AAV8 genome.

748. The nucleic acid of embodiment 743, wherein the AAV genome is an AAV9 genome.

749. The nucleic acid of embodiment 743, wherein the AAV genome is an AAVrh8r genome.

750. The nucleic acid of embodiment 743, wherein the AAV genome is an AAVrh10 genome.

751. A plurality of nucleic acids comprising separate nucleic acids encoding the Type II Cas protein and gRNA of the system of any one of embodiments 614 to 698.

752. The plurality of nucleic acid of embodiment 751, wherein the separate nucleic acids encoding the Type II Cas protein and gRNA are plasmids.

753. The plurality of nucleic acids of embodiment 751, wherein the separate nucleic acids encoding the Type II Cas protein and gRNA are viral genomes.

754. The plurality of nucleic acids of embodiment 753, wherein the viral genomes are adeno-associated virus (AAV) genomes.

755. The plurality of nucleic acids of embodiment 754, wherein the AAV genomes the encoding the Type II Cas protein and gRNA are independently an AAV2, AAV5, AAV7m8, AAV8, AAV9, AAVrh8r, or AAVrh10 genome.

756. A Type II Cas protein according to any one of embodiments 1 to 255, a gRNA according to any one of embodiments 256 to 611, a combination of gRNAs according to any one of embodiments 612 to 613, a system according to of any one of embodiments 614 to 698, a nucleic acid according to any one of embodiments 699 to 750, a plurality of nucleic acids according to of any one of embodiments 751 to 755, particle according to any one of embodiments 763 to 778, or pharmaceutical composition according to embodiment 779 for use in a method of editing a human genomic sequence.

757. The Type II Cas protein, gRNA, system, nucleic acid, a plurality of nucleic acids, particle, or pharmaceutical composition for use according to embodiment 756, wherein the human genomic sequence is a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence.

758. The Type II Cas protein, gRNA, combination of gRNAs, system, nucleic acid, a plurality of nucleic acids, particle, or pharmaceutical composition for use according to embodiment 757, wherein the human genomic sequence is a RHO genomic sequence, optionally wherein the RHO genomic sequence has a pathogenic mutation.

759. The Type II Cas protein, gRNA, combination of gRNAs, system, nucleic acid, a plurality of nucleic acids, particle, or pharmaceutical composition for use according to embodiment 757, wherein the human genomic sequence is a TRAC genomic sequence, optionally wherein the human genomic sequence is in a T cell.

760. The Type II Cas protein, gRNA, combination of gRNAs, system, nucleic acid, a plurality of nucleic acids, particle, or pharmaceutical composition for use according to embodiment 757, wherein the human genomic sequence is a B2M genomic sequence, optionally wherein the human genomic sequence is in a T cell.

761. The Type II Cas protein, gRNA, combination of gRNAs, system, nucleic acid, a plurality of nucleic acids, particle, or pharmaceutical composition for use according to embodiment 757, wherein the human genomic sequence is a PD1 genomic sequence, optionally wherein the human genomic sequence is in a T cell.

762. The Type II Cas protein, gRNA, combination of gRNAs, system, nucleic acid, a plurality of nucleic acids, particle, or pharmaceutical composition for use according to embodiment 757, wherein the human genomic sequence is a LAG3 genomic sequence, optionally wherein the human genomic sequence is in a T cell.

763. A particle comprising a Type II Cas protein according to any one of embodiments 1 to 255, a gRNA according to any one of embodiments 256 to 611, a combination of gRNAs according to any one of embodiments 612 to 613, a system according to of any one of embodiments 614 to 698, a nucleic acid according to any one of embodiments 699 to 750, or a plurality of nucleic acids according to of any one of embodiments 751 to 755.

764. The particle of embodiment 763, which is a lipid nanoparticle, a vesicle, a gold nanoparticle, a viral-like particle (VLP) or a viral particle.

765. The particle of embodiment 764, which is a lipid nanoparticle.

766. The particle of embodiment 764, which is a vesicle.

767. The particle of embodiment 764, which is a gold nanoparticle.

768. The particle of embodiment 764, which is a viral-like particle (VLP).

769. The particle of embodiment 764, which is a viral particle.

770. The particle of embodiment 769, which is an adeno-associated virus (AAV) particle.

771. The particle of embodiment 770, wherein the AAV particle is an AAV2, AAV5, AAV7m8, AAV8, AAV9, AAVrh8r, or AAVrh10 particle.

772. The particle of embodiment 771, wherein the AAV particle is an AAV2 particle.

773. The particle of embodiment 771, wherein the AAV particle is an AAV5 particle.

774. The particle of embodiment 771, wherein the AAV particle is an AAV7m8 particle.

775. The particle of embodiment 771, wherein the AAV particle is an AAV8 particle.

776. The particle of embodiment 771, wherein the AAV particle is an AAV9 particle.

777. The particle of embodiment 771, wherein the AAV particle is an AAVrh8r particle.

778. The particle of embodiment 771, wherein the AAV particle is an AAVrh10 particle.

779. A pharmaceutical composition comprising a Type II Cas protein according to any one of embodiments 1 to 255, a gRNA according to any one of embodiments 256 to 611, a combination of gRNAs according to any one of embodiments 612 to 613, a system according to of any one of embodiments 614 to 698, a nucleic acid according to any one of embodiments 699 to 750, or a plurality of nucleic acids according to of any one of embodiments 751 to 755, or a particle according to any one of embodiments 758 to 778 and at least one pharmaceutically acceptable excipient.

780. A cell comprising a Type II Cas protein according to any one of embodiments 1 to 255, a gRNA according to any one of embodiments 256 to 545, a system according to of any one of embodiments 546 to 698, a nucleic acid according to any one of embodiments 699 to 750, or a plurality of nucleic acids according to of any one of embodiments 751 to 755, or a particle according to any one of embodiments 763 to 778.

781. The cell of embodiment 780, which is a human cell.

782. The cell of embodiment 780 or embodiment 781, wherein the cell is a hematopoietic progenitor cell.

783. The cell of any one of embodiments 780 to 782, which is a stem cell.

784. The cell of embodiment 783, wherein the stem cell is a hematopoietic stem cell (HSC), a pluripotent stem cell, or an induced pluripotent stem cell (iPS).

785. The cell of embodiment 784, wherein the stem cell is an embryonic stem cell.

786. The cell of any one of embodiments 780 to 785, which is an ex vivo cell.

787. A population of cells according to any one embodiments 780 to 786.

788. A method for altering a cell, the method comprising contacting the cell with a Type II Cas protein according to any one of embodiments 1 to 255, a gRNA according to any one of embodiments 256 to 611, a combination of gRNAs according to any one of embodiments 612 to 613, a system according to of any one of embodiments 614 to 698, a nucleic acid according to any one of embodiments 699 to 750, or a plurality of nucleic acids according to of any one of embodiments 751 to 755, a particle according to any one of embodiments 763 to 778, or a pharmaceutical composition according to embodiment 779.

789. The method of embodiment 788, which comprises contacting the cell with the Type II Cas protein of any one of embodiments 1 to 255.

790. The method of embodiment 788, which comprises contacting the cell with the gRNA of any one of embodiments 256 to 611.

791. The method of embodiment 788, which comprises contacting the cell with the combination of gRNAs according to any one of embodiments 612 to 613.

792. The method of embodiment 788, which comprises contacting the cell with the system of any one of embodiments 614 to 698.

793. The method of embodiment 792, which comprises electroporation of the cell prior to contacting the cell with the system.

794. The method of embodiment 792, which comprises lipid-mediated delivery of the system to the cell, optionally wherein the lipid-mediated delivery is cationic lipid-mediated delivery.

795. The method of embodiment 792, which comprises polymer-mediated delivery of the system to the cell.

796. The method of embodiment 792, which comprises delivery of the system to the cell by lipofection.

797. The method of embodiment 792, which comprises delivery of the system to the cell by nucleofection.

798. The method of embodiment 788, which comprises contacting the cell with the nucleic acid of any one of embodiments 699 to 750.

799. The method of embodiment 788, which comprises contacting the cell with the plurality of nucleic acids of any one of embodiments 751 to 755.

800. The method of embodiment 788, which comprises contacting the cell with the particle of any one of embodiments 763 to 778.

801. The method of embodiment 788, which comprises contacting the cell with the pharmaceutical composition of embodiment 779.

802. The method of any one of embodiments 788 to 801, wherein the contacting alters a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence

803. The method of any one of embodiments 788 to 801, wherein the contacting alters a CCR5, EMX1, Fas, FANCF, HBB, ZSCAN, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, or BCR genomic sequence.

804. The method of any one of embodiments 788 to 801, wherein the contacting alters a RHO genomic sequence.

805. The method of embodiment 804, wherein the cell has a RHO allele having a pathogenic mutation and the contacting alters the RHO allele having the pathogenic mutation, optionally wherein the alteration is a deletion.

806. The method of embodiment 805, wherein the cell is a cell from a subject having a RHO allele with the pathogenic mutation or a progeny of such cell.

807. The method of embodiment 806, wherein the subject is heterozygous for the rs7984 SNP and the subject is heterozygous for the pathogenic mutation.

808. The method of embodiment 807, which further comprises a step of genotyping the subject to determine which allele of the rs7984 SNP is in phase with the pathogenic mutation.

809. The method of any one of embodiments 788 to 801, wherein the contacting alters a B2M genomic sequence.

810. The method of any one of embodiments 788 to 801, wherein the contacting alters a TRAC genomic sequence.

811. The method of any one of embodiments 788 to 801, wherein the contacting alters a PD1 genomic sequence.

812. The method of any one of embodiments 788 to 801, wherein the contacting alters a LAG3 genomic sequence.

813. The method of any one of embodiments 788 to 812, wherein the cell is a human cell. 814. The method of any one of embodiments 788 to 813, wherein the cell is a hematopoietic progenitor cell.

815. The method of any one of embodiments 788 to 814, wherein the cell is a stem cell.

816. The method of embodiment 815, wherein the stem cell is a hematopoietic stem cell (HSC), a pluripotent stem cell, or an induced pluripotent stem cell (iPS).

817. The method of embodiment 816, wherein the stem cell is an embryonic stem cell.

818. The method of any one of embodiments 788 to 813, wherein the cell is a retinal cell.

819. The method of any one of embodiments 788 to 813, wherein the cell is a photoreceptor cell.

820. The method of any one of embodiments 788 to 813, wherein the cell is a T cell.

821. The method of any one of embodiments 788 to 820, wherein the contacting is in vitro.

822. The method of embodiment 821, further comprising transplanting the cell to a subject.

823. The method of any one of embodiments 788 to 820, wherein the contacting is in vivo in a subject.

824. A cell or population of cells produced by the method of any one of embodiments 788 to 818.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A Type II Cas protein comprising an amino acid sequence having at least 50% sequence identity to:
(a) the amino acid sequence of a RuvC-I domain of a reference protein sequence;
(b) the amino acid sequence of a RuvC-II domain of a reference protein sequence;
(c) the amino acid sequence of a RuvC-III domain of a reference protein sequence;
(d) the amino acid sequence of a BH domain of a reference protein sequence;
(e) the amino acid sequence of a REC domain of a reference protein sequence;
(f) the amino acid sequence of a HNH domain of a reference protein sequence;
(g) the amino acid sequence of a WED domain of a reference protein sequence;
(h) the amino acid sequence of a PID domain of a reference protein sequence; or
(i) the amino acid sequence of the full length of a reference protein sequence;
wherein the reference protein sequence is SEQ ID NO:1, SEQ ID NO:2, SEQ ID NO:7, SEQ ID NO:8, SEQ ID NO:13, SEQ ID NO:14, SEQ ID NO:19, SEQ ID NO:20, SEQ ID NO:25, SEQ ID NO:26, SEQ ID NO:31, SEQ ID NO:32, SEQ ID NO:37, SEQ ID NO:38, SEQ ID NO:43, SEQ ID NO:44, SEQ ID NO:49, SEQ ID NO:50, SEQ ID NO:55, or SEQ ID NO:56.

2. The Type II Cas protein of claim 1, wherein the amino acid sequence of the Type II Cas protein comprises an amino acid sequence that is at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, or is at least 99% identical to the full length of the reference protein sequence.

3. The Type II Cas protein of claim 1, wherein the amino acid sequence of the Type II Cas protein comprises an amino acid sequence that is identical to the full length of the reference protein sequence.

4. The Type II Cas protein of any one of claims 1 to 3, which is a fusion protein.

5. The Type II Cas protein of claim 4, which comprises one or more nuclear localization signals, such as two or more nuclear localization signals, and which optionally comprises an N-terminal nuclear localization signal and/or a C-terminal nuclear localization signal.

6. The Type II Cas protein of claim 5, wherein the amino acid sequence of one or more of the nuclear localization signals comprises the amino acid sequence KRTADGSEFESPKKKRKV (SEQ ID NO:109), PKKKRKV (SEQ ID NO:110), PKKKRRV (SEQ ID NO:111), KRPAATKKAGQAKKKK (SEQ ID NO:112), YGRKKRRQRRR (SEQ ID NO:113), RKKRRQRRR (SEQ ID NO:114), PAAKRVKLD (SEQ ID NO:115), RQRRNELKRSP (SEQ ID NO:116), VSRKRPRP (SEQ ID NO:117), PPKKARED (SEQ ID NO:118), PQPKKKPL (SEQ ID NO:119), SALIKKKKKMAP (SEQ ID NO:120), PKQKKRK (SEQ ID NO:121), RKLKKKIKKL (SEQ ID NO:122), REKKKFLKRR (SEQ ID NO:123), KRKGDEVDGVDEVAKKKSKK (SEQ ID NO:124), RKCLQAGMNLEARKTKK (SEQ ID NO:125), NQSSNFGPMKGGNFGGRSSGPYGGGGQYFAKPRNQGGY (SEQ ID NO:126), or RMRIZFKNKGKDTAELRRRRVEVSVELRKAKKDEQILKRRNV (SEQ ID NO:127).

7. The Type II Cas protein of claim 5 or claim 6, wherein the amino acid sequence of each nuclear localization signal is the same.

8. The Type II Cas protein of any one of claims 4 to 7, which comprises a fusion partner which is a DNA, RNA or protein modification enzyme, optionally wherein the DNA, RNA or protein modification enzyme is an adenosine deaminase, a cytidine deaminase, a reverse transcriptase, a guanosyl transferase, a DNA methyltransferase, a RNA methyltransferase, a DNA demethylase, a RNA demethylase, a dioxygenase, a polyadenylate polymerase, a pseudouridine synthase, an acetyltransferase, a deacetylase, a ubiquitin-ligase, a deubiquitinase, a kinase, a phosphatase, a NEDD8-ligase, a de-NEDDylase, a SUMO-ligase, a deSUMOylase, a histone deacetylase, a histone acetyltransferase, a histone methyltransferase, or a histone demethylase.

9. The Type II Cas protein of any one of claims 1 to 8, which comprises a tag, e.g., a SV5 tag, optionally wherein the SV5 tag comprises the amino acid sequence GKPIPNPLLGLDST (SEQ ID NO:128) or IPNPLLGLD (SEQ ID NO:129).

10. The Type II Cas protein of any one of claims 1 to 9, whose amino acid sequence comprises the amino acid sequence of SEQ ID NO:1, SEQ ID NO:2, or SEQ ID NO:3.

11. The Type II Cas protein any one of claims 1 to 9, whose amino acid sequence comprises the amino acid sequence of SEQ ID NO:7, SEQ ID NO:8, or SEQ ID NO:9.

12. The Type II Cas protein of any one of claims 1 to 9, whose amino acid sequence comprises the amino acid sequence of SEQ ID NO:13, SEQ ID NO:14, or SEQ ID NO:15.

13. The Type II Cas protein of any one of claims 1 to 9, whose amino acid sequence comprises the amino acid sequence of SEQ ID NO:19, SEQ ID NO:20, or SEQ ID NO:21.

14. The Type II Cas protein of any one of claims 1 to 9, whose amino acid sequence comprises the amino acid sequence of SEQ ID NO:25, SEQ ID NO:26, or SEQ ID NO:27.

15. The Type II Cas protein of any one of claims 1 to 9, whose amino acid sequence comprises the amino acid sequence of SEQ ID NO:31, SEQ ID NO:32, or SEQ ID NO:33.

16. The Type II Cas protein of any one of claims 1 to 9, whose amino acid sequence comprises the amino acid sequence of SEQ ID NO:37, SEQ ID NO:38, or SEQ ID NO:39.

17. The Type II Cas protein of any one of claims 1 to 9, whose amino acid sequence comprises the amino acid sequence of SEQ ID NO:43, SEQ ID NO:44, or SEQ ID NO:45.

18. The Type II Cas protein of any one of claims 1 to 9, whose amino acid sequence comprises the amino acid sequence of SEQ ID NO:49, SEQ ID NO:50, or SEQ ID NO:51.

19. The Type II Cas protein of any one of claims 1 to 9, whose amino acid sequence comprises the amino acid sequence of SEQ ID NO:55, SEQ ID NO:56, or SEQ ID NO:57.

20. A Type II Cas protein whose amino acid sequence is identical to a Type II Cas protein of any one of claims 1 to 19 except for one or more amino acid substitutions relative to the reference sequence that provide nickase activity or that render the Type II Cas protein catalytically inactive.

21. A guide RNA (gRNA) molecule for editing a human RHO gene, the gRNA comprising a spacer whose nucleotide sequence comprises 15 or more consecutive nucleotides of a reference sequence or comprises a nucleotide sequence that is at least 85% identical to the reference sequence, wherein the reference sequence is
(a)
(SEQ ID NO: 287)
UUGUGGCUGACCCGYGGCUGCUC;
(b)
(SEQ ID NO: 288)
UUGUGGCUGACCCGUGGCUGCUC;
(c)
(SEQ ID NO: 289)
UUGUGGCUGACCCGCGGCUGCUC;
(d)
(SEQ ID NO: 290)
CUUGUGGCUGACCCGYGGCUGCU;
(e)
(SEQ ID NO: 291)
CUUGUGGCUGACCCGUGGCUGCU;
(f)
(SEQ ID NO: 292)
CUUGUGGCUGACCCGCGGCUGCU;
(g)
(SEQ ID NO: 293)
GGCCCUUGUGGCUGACCCGYGGC;
(h)
(SEQ ID NO: 294)
GGCCCUUGUGGCUGACCCGUGGC;
(i)
(SEQ ID NO: 295)
GGCCCUUGUGGCUGACCCGCGGC;
(j)
(SEQ ID NO: 296)
CUUGGGUGGGAGCAGCCRCGGGU;
(k)
(SEQ ID NO: 297)
CUUGGGUGGGAGCAGCCACGGGU;
(l)
(SEQ ID NO: 298)
CUUGGGUGGGAGCAGCCGCGGGU;
(m)
(SEQ ID NO: 299)
GAGCAGCCRCGGGUCAGCCACAA;
(n)
(SEQ ID NO: 300)
GAGCAGCCACGGGUCAGCCACAA;
(o)
(SEQ ID NO: 301)
GAGCAGCCGCGGGUCAGCCACAA;
(p)
(SEQ ID NO: 302)
CAUGGCUGUGGCCCUUGUGGCUG;
(q)
(SEQ ID NO: 303)
GUGGGAGCAGCCRCGGGUCAGCC;
(r)
(SEQ ID NO: 304)
GUGGGAGCAGCCACGGGUCAGCC;
(s)
(SEQ ID NO: 305)
GUGGGAGCAGCCGCGGGUCAGCC;
(t)
(SEQ ID NO: 306)
GGCUGACCCGYGGCUGCUCCCAC;
(u)
(SEQ ID NO: 307)
GGCUGACCCGUGGCUGCUCCCAC;
or
(v)
(SEQ ID NO: 308)
GGCUGACCCGCGGCUGCUCCCAC,
where Y is U or C and R is A or G.

22. A guide RNA (gRNA) molecule for editing a human B2M gene, the gRNA comprising a spacer whose nucleotide sequence comprises 15 or more consecutive nucleotides of a reference sequence or comprises a nucleotide sequence that is at least 85% identical to the reference sequence, wherein the reference sequence is
(a)
(SEQ ID NO: 309)
CAGAAAGAGAGAGUAGCGCGAGC;
(b)
(SEQ ID NO: 310)
CACGUCAUCCAGCAGAGAAUGGA;
(c)
(SEQ ID NO: 311)
CAUUCUUCAGUAAGUCAACUUCA;
(d)
(SEQ ID NO: 312)
AGCAUUCGGGCCGAGAUGUCUCG;
(e)
(SEQ ID NO: 313)
GAGAUGUCUCGCUCCGUGGCCUU;
(f)
(SEQ ID NO: 314)
GGAUAGCCUCCAGGCCAGAAAGA;
(g)
(SEQ ID NO: 315)
UUGACUUUCCAUUCUCUGCUGGA;
or
(h)
(SEQ ID NO: 316)
GGAAAGUCAAAUUUCCUGAAUUG.

23. A guide RNA (gRNA) molecule for editing a human TRAC gene, the gRNA comprising a spacer whose nucleotide sequence comprises 15 or more consecutive nucleotides of a reference sequence or comprises a nucleotide sequence that is at least 85% identical to the reference sequence, wherein the reference sequence is
(a)
(SEQ ID NO: 317
UUUGUCUGUGAUAUACACAUCAG);
(b)
(SEQ ID NO: 318)
GUAAGGAUUCUGAUGUGUAUAUCA;
(c)
(SEQ ID NO: 319)
GGCCACAGCACUGUUGCUCUUGA;
(d)
(SEQ ID NO: 320)
GAGCAACAGUGCUGUGGCCUGGA;
(e)
(SEQ ID NO: 321)
GGAGCAACAAAUCUGACUUUGCA;
(f)
(SEQ ID NO: 322)
AAUGUGUCACAAAGUAAGGAUUC;
(g)
(SEQ ID NO: 323)
UCUGAUGUGUAUAUCACAGACAA;
(h)
(SEQ ID NO: 324)
GCCUGGAGCAACAAAUCUGACUUU;
(i)
(SEQ ID NO: 325)
GGCGUUUGCACAUGCAAAGUCAG;
(j)
(SEQ ID NO: 326)
GAAGAAGGUGUCUUCUGGAAUAA;
or
(k)
(SEQ ID NO: 327)
GCUGCCCUUACCUGGGCUGGGGAA.

24. A guide RNA (gRNA) molecule for editing a human PD1 gene, the gRNA comprising a spacer whose nucleotide sequence comprises 15 or more consecutive nucleotides of a reference sequence or comprises a nucleotide sequence that is at least 85% identical to the reference sequence, wherein the reference sequence is
(a)
(SEQ ID NO: 328)
UGGGCUGGCGGCCAGGAUGGUUC;
(b)
(SEQ ID NO: 329)
CCGCCCAGACGACUGGCCAGGGC;
(c)
(SEQ ID NO: 330)
CAGGCGCCCUGGCCAGUCGUCUG;
(d)
(SEQ ID NO: 331)
AGAACCAUCCUGGCCGCCAGCCC;
(e)
(SEQ ID NO: 332)
CUAAGAACCAUCCUGGCCGCCAG;
(f)
(SEQ ID NO: 333)
GCUGGCGGCCAGGAUGGUUCUUAG;
(g)
(SEQ ID NO: 334)
ACAACUGGGCUGGCGGCCAGGAU;
(h)
(SEQ ID NO: 335)
GUGGGGCUGCUCCAGGCAUGCAG;
or
(i)
(SEQ ID NO: 336)
GGCCGCCAGCCCAGUUGUAGCAC.

25. The gRNA of any one of claims 21 to 24, which comprises a spacer that is 15 to 30 nucleotides in length, 18 to 30 nucleotides in length, 20 to 28 nucleotides in length, 22 to 26 nucleotides in length, 23 to 25 nucleotides in length, 22 to 25 nucleotides in length, 15 to 25 nucleotides in length, 16 to 24 nucleotides in length, 17 to 23 nucleotides in length, 18 to 22 nucleotides in length, 19 to 21 nucleotides in length, 25 nucleotides in length, 24 nucleotides in length, 23 nucleotides in length, 22 nucleotides in length, 21 nucleotides in length, or 20 nucleotides in length.

26. The gRNA of any one of claims 21 to 25, wherein the spacer comprises the reference sequence.

27. The gRNA of any one of claims 21 to 26, which is a single guide RNA (sgRNA).

28. A gRNA comprising a spacer and a sgRNA scaffold, wherein:
(a) the spacer is positioned 5′ to the sgRNA scaffold; and
(b) the nucleotide sequence of the sgRNA scaffold comprises a nucleotide sequence that is at least 50% identical to a reference scaffold sequence, wherein the reference scaffold sequence is any one of SEQ ID NOS:77-92.

29. The gRNA of claim 28, wherein the sgRNA scaffold comprises a nucleotide sequence that is 100% identical to the reference scaffold sequence.

30. The gRNA of claim 28, wherein the nucleotide sequence of the sgRNA scaffold comprises the nucleotide sequence of any one of SEQ ID NOS:93-108.

31. The gRNA of any one of claims 28 to 30, wherein the target mammalian genomic sequence is a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence.

32. The gRNA of any one of claims 28 to 30, wherein the nucleotide sequence of the spacer comprises a sequence selected from SEQ ID NOs:287-336.

33. A system comprising the Type II Cas protein of any one of claims 1 to 20 and a guide RNA (gRNA) comprising a spacer sequence.

34. A nucleic acid encoding the Type II Cas protein of any one of claims 1 to 20, optionally wherein the nucleotide sequence encoding the Type II Cas protein is operably linked to a promoter that is heterologous to the Type II Cas protein.

35. The nucleic acid of claim 34, wherein the nucleotide sequence encoding the Type II Cas protein is codon optimized for expression in human cells.

36. A nucleic acid encoding the gRNA of any one of claims 21 to 32.

37. A plurality of nucleic acids comprising separate nucleic acids encoding the Type II Cas protein and gRNA of the system of claim 33.

38. A particle comprising a Type II Cas protein according to any one of claims 1 to 20, a gRNA according to any one of claims 21 to 32, a system according to claim 33, a nucleic acid according to any one of claims 34 to 36, or a plurality of nucleic acids according to claim 37.

39. A pharmaceutical composition comprising a Type II Cas protein according to any one of claims 1 to 20, a gRNA according to any one of claims 21 to 32, a system according to claim 33, a nucleic acid according to any one of claims 34 to 36, a plurality of nucleic acids according to claim 37, or a particle according to claim 38 and at least one pharmaceutically acceptable excipient.

40. A Type II Cas protein according to any one of claims 1 to 20, a gRNA according to any one of claims 21 to 32, a system according to claim 33, a nucleic acid according to any one of claims 34 to 36, a plurality of nucleic acids according to claim 37, a particle according to claim 38, or pharmaceutical composition according to claim 39 for use in a method of editing a human genomic sequence.

41. The Type II Cas protein, gRNA, system, nucleic acid, a plurality of nucleic acids, particle, or pharmaceutical composition for use according to claim 40, wherein the human genomic sequence is a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence.

42. The Type II Cas protein, gRNA, combination of gRNAs, system, nucleic acid, a plurality of nucleic acids, particle, or pharmaceutical composition for use according to claim 41, wherein the human genomic sequence is a RHO genomic sequence, optionally wherein the RHO genomic sequence has a pathogenic mutation.

43. The Type II Cas protein, gRNA, combination of gRNAs, system, nucleic acid, a plurality of nucleic acids, particle, or pharmaceutical composition for use according to claim 41, wherein the human genomic sequence is a TRAC, B2M, PD1, or LAG3 genomic sequence, optionally wherein the human genomic sequence is in a T cell.

44. An ex vivo human cell comprising a Type II Cas protein according to any one of claims 1 to 20, a gRNA according to any one of claims 21 to 32, a system according to claim 33, a nucleic acid according to any one of claims 34 to 36, a plurality of nucleic acids according to claim 37, or a particle according to claim 38.


SEQUENCE LISTING (455 sequences)

SEQ ID NO: 1 - protein, 1072 aa, synthetic construct
EKRNNGLVLGLDIGVTSVGYGIVNRETGEAVDFGVRLFSEWDPANNQNRRAKRSGRRLRSRRKNRLQDMKKFLVDQGMIPSDYAFHLDPYALRVKGLSEKLTDEELATVLYNLAKLRGSSLETVEDTDDTKSDDSLSTKNNLRINDALIKSGKYVCQIQQERLSEKEKLRGHINNFRTKDYVSELKQILSNQGKSKDFNDYVENLIVRRRDFSDGPGSQKSPTPYGRWLTKDQEEPIDLIEKMRGKCSYYPDQLRCAKMAYKADLFNLLNDLNNLSVKGEKLERDQKEQVIEYVNEKGSIKPKQLAKLLGLSDIDDISGFRIDKSDKPILTEFKGYIKVKKVLENSGLYHDHELVDQIIDVLTRDKVVERRANEIRKLSNKFSEEEIEALANLTQISGYHSMSAKAINEFNKEMLISPLNQMQIITNRGNQNDRFNKLKGMKEIHADDTAILSPVAKRAQREAMKVVNRLRELYGEFDSIVVETTRAKNSQEQKTKIKESQAFFEKEKKRAQQIVDENKYNLTVNAKLIQKLRLYTEQNYKTAYALVPIDLDLLISDPTAFEVDHIIPISISYDDSFSNKVLVTRWENQIKGNLTPLQAHKNHKFGEYSDLASYIANVNDLRKMNGMRYEKKKNLLLSDEDLTKFDTQKKFIARNIVDTAYAERVVLNTLQDYFRANEISTVVNTVKGNLTHMFRNKIHLDKERSDIDTGDAHHAIDALIIASLKVFRRFGEILDKRPETIIDEETGEVLEIIEDKKFFDPRYIEIVHKLSLIEKDVTKFSYKIDTKPNRQVADETIYSTRNINGEDLVVKKYKNIYEEKFVTLANDILENNVEKYLMYRNSPETFKYISDIILYYAKEYSDDEKLMKNGLFVKNPLNKYYEEHHEYLHKPSKHNNGPSIISMKYTDGRLGNKIDISKNYSVDTEKKKVVLQQISPYRTDFYYSPIEGYRFVTIRYSNVYYSSTQGLYIIEKDWYKSQKKSKKIDDSFEFCFSMHRNEIIRIKKEDEDMMWRFTATNNDSTNKIEVKPIGHKINKQLILAIGKKIILLEKYATDVCGNLYKVTNQELKLEFK

SEQ ID NO: 2 - protein, 1073 aa, synthetic construct
MEKRNNGLVLGLDIGVTSVGYGIVNRETGEAVDFGVRLFSEWDPANNQNRRAKRSGRRLRSRRKNRLQDMKKFLVDQGMIPSDYAFHLDPYALRVKGLSEKLTDEELATVLYNLAKLRGSSLETVEDTDDTKSDDSLSTKNNLRINDALIKSGKYVCQIQQERLSEKEKLRGHINNFRTKDYVSELKQILSNQGKSKDFNDYVENLIVRRRDFSDGPGSQKSPTPYGRWLTKDQEEPIDLIEKMRGKCSYYPDQLRCAKMAYKADLFNLLNDLNNLSVKGEKLERDQKEQVIEYVNEKGSIKPKQLAKLLGLSDIDDISGFRIDKSDKPILTEFKGYIKVKKVLENSGLYHDHELVDQIIDVLTRDKVVERRANEIRKLSNKFSEEEIEALANLTQISGYHSMSAKAINEFNKEMLISPLNQMQIITNRGNQNDRFNKLKGMKEIHADDTAILSPVAKRAQREAMKVVNRLRELYGEFDSIVVETTRAKNSQEQKTKIKESQAFFEKEKKRAQQIVDENKYNLTVNAKLIQKLRLYTEQNYKTAYALVPIDLDLLISDPTAFEVDHIIPISISYDDSFSNKVLVTRWENQIKGNLTPLQAHKNHKFGEYSDLASYIANVNDLRKMNGMRYEKKKNLLLSDEDLTKFDTQKKFIARNIVDTAYAERVVLNTLQDYFRANEISTVVNTVKGNLTHMFRNKIHLDKERSDIDTGDAHHAIDALIIASLKVFRRFGEILDKRPETIIDEETGEVLEIIEDKKFFDPRYIEIVHKLSLIEKDVTKFSYKIDTKPNRQVADETIYSTRNINGEDLVVKKYKNIYEEKFVTLANDILENNVEKYLMYRNSPETFKYISDIILYYAKEYSDDEKLMKNGLFVKNPLNKYYEEHHEYLHKPSKHNNGPSIISMKYTDGRLGNKIDISKNYSVDTEKKKVVLQQISPYRTDFYYSPIEGYRFVTIRYSNVYYSSTQGLYIIEKDWYKSQKKSKKIDDSFEFCFSMHRNEIIRIKKEDEDMMWRFTATNNDSTNKIEVKPIGHKINKQLILAIGKKIILLEKYATDVCGNLYKVTNQELKLEFK

SEQ ID NO: 3 - protein, 1122 aa, synthetic construct
MIPNPLLGLDKRTADGSEFESPKKKRKVGSEKRNNGLVLGLDIGVTSVGYGIVNRETGEAVDFGVRLFSEWDPANNQNRRAKRSGRRLRSRRKNRLQDMKKFLVDQGMIPSDYAFHLDPYALRVKGLSEKLTDEELATVLYNLAKLRGSSLETVEDTDDTKSDDSLSTKNNLRINDALIKSGKYVCQIQQERLSEKEKLRGHINNFRTKDYVSELKQILSNQGKSKDFNDYVENLIVRRRDFSDGPGSQKSPTPYGRWLTKDQEEPIDLIEKMRGKCSYYPDQLRCAKMAYKADLFNLLNDLNNLSVKGEKLERDQKEQVIEYVNEKGSIKPKQLAKLLGLSDIDDISGFRIDKSDKPILTEFKGYIKVKKVLENSGLYHDHELVDQIIDVLTRDKVVERRANEIRKLSNKFSEEEIEALANLTQISGYHSMSAKAINEFNKEMLISPLNQMQIITNRGNQNDRFNKLKGMKEIHADDTAILSPVAKRAQREAMKVVNRLRELYGEFDSIVVETTRAKNSQEQKTKIKESQAFFEKEKKRAQQIVDENKYNLTVNAKLIQKLRLYTEQNYKTAYALVPIDLDLLISDPTAFEVDHIIPISISYDDSFSNKVLVTRWENQIKGNLTPLQAHKNHKFGEYSDLASYIANVNDLRKMNGMRYEKKKNLLLSDEDLTKFDTQKKFIARNIVDTAYAERVVLNTLQDYFRANEISTVVNTVKGNLTHMFRNKIHLDKERSDIDTGDAHHAIDALIIASLKVFRRFGEILDKRPETIIDEETGEVLEIIEDKKFFDPRYIEIVHKLSLIEKDVTKFSYKIDTKPNRQVADETIYSTRNINGEDLVVKKYKNIYEEKFVTLANDILENNVEKYLMYRNSPETFKYISDIILYYAKEYSDDEKLMKNGLFVKNPLNKYYEEHHEYLHKPSKHNNGPSIISMKYTDGRLGNKIDISKNYSVDTEKKKVVLQQISPYRTDFYYSPIEGYRFVTIRYSNVYYSSTQGLYIIEKDWYKSQKKSKKIDDSFEFCFSMHRNEIIRIKKEDEDMMWRFTATNNDSTNKIEVKPIGHKINKQLILAIGKKIILLEKYATDVCGNLYKVTNQELKLEFKSRKRTADGSEFESPKKKRKV

SEQ ID NO: 4 - DNA, 3221 bp, synthetic construct
  source 1..3221: mol_type = other DNA
atggagaaaagaaataatggtttagttttagggttagacatcggtgttacttcagttggttatggaattgtaaatcgtgaaactggagaagcagttgattttggtgtgcgcttatttagcgaatgggatccagcaaataaccaaaatagacgtgcaaagagaagtggcagaagacttcgctctagaaggaaaaatcgattacaagatatgaaaaaatttcttgttgatcaaggatgattccttctgattatgcttttcatctagatccatatgcattaagagttaaaggcttatcagagaaattgacagatgaagaacttgcaacagttctatacaacttagcaaaattacgtggaagtagtttggagactgtagaagatacagatgatactaagagtgatgattctctaagtacgaaaaataatttaagaattaatgatgcattaatcaagagtggaaaatatgtttgtcaaattcagcaagaaagattatctgaaaaggaaaagttacgcggacatattaacaacttccgtacaaaggattatgtttcagaattaaaacaaatcttatcaaatcagggtaagtcaaaagattttaatgattatgtagaaaacttaatagtgcgtagaagagatttttctgacggacctggttcccagaaatctcctacaccttatggacgttggttaacaaaagatcaagaagaaccaatagacttgattgagaagatgagaggtaagtgctcatactatcctgatcaattacgttgtgcaaaaatggcttataaagctgatttatttaatttgctaaacgacttgaataacttaagtgtcaaaggtgaaaaacttgaaagagatcaaaaagaacaagtcattgaatacgttaatgaaaagggttctatcaagccaaaacaattagccaaactattaggactcagtgatattgatgatatttctggttttagaatagataagagtgataagccaatactcactgaattcaaaggctatataaaagttaagaaggttcttgagaattcgggtttgtatcatgaccatgaacttgttgatcagataattgatgttttaacacgtgataaggtagttgaaagacgtgcgaatgaaattcgtaaactatcaaataagtttagtgaagaggagatagaggcattagctaacctaacacagatttctggataccattctatgtcagcaaaggcaattaatgagtttaataaagagatgcttatttcaccattaaaccagatgcaaattattacgaatagaggtaatcagaacgaccgctttaataaactaaaaggaatgaaagagatccatgcagatgatactgcaattttaagtccagttgctaagagagcacaaagagaagcaatgaaagtcgttaatagacttagagaactctatggtgaatttgatagtattgttgtagagacaacacgtgcaaaaaatagccaggaacaaaagacaaaaattaaagagtcacaagctttctttgaaaaagaaaagaaacgtgcccaacaaatagtcgatgaaaataaatataatctaacagttaatgcaaagctaattcagaagttacgtttatatacagaacagaattataagactgcgtatgcactagtgccaattgatttagatttattaattagtgatccgacagcttttgaggtagatcatattattccaatctctatttcctatgatgattcgttctcaaataaggttcttgtaactcgttgggagaatcagattaaaggtaatctaacaccactacaggcacataagaatcataagtttggagagtattcagatttagcaagttatattgcaaacgtcaatgacttacgcaagatgaatggaatgcgatatgagaaaaagaaaaatctcttgttatcagatgaggatttgacgaagtttgatacacaaaagaaatttattgctagaaatattgtggatactgcatatgcagaaagagttgttttaaatacattgcaagactacttccgtgctaatgaaatttctactgttgtaaataccgtaaaaggtaatttaacgcacatgtttagaaataagattcatcttgataaggagagaagtgatattgatactggtgatgcacatcacgctattgatgcattgattattgcttccttaaaggtcttccgccgtttcggagaaatccttgataagcgaccagaaacaattattgatgaagaaacaggggaagttttagaaattattgaggataagaaattctttgatccacggtatatagaaattgttcataaactttcactgatagaaaaggatgtaacgaagttttcttacaagattgatactaaacctaatcgacaagtagctgacgaaacaatctattctacaagaaatattaatggggaagatcttgttgttaagaaatataaaaacatttacgaagaaaagtttgttacattagcaaatgatattttagaaaataatgttgaaaaatatctgatgtatcgcaatagtccagaaacatttaagtatatatctgatattattctgtattatgcaaaagaatatagtgacgatgagaaacttatgaagaatggattatttgtaaagaacccattaaataaatattacgaagaacatcatgagtatctacataagccatcgaaacataataatggaccatcgattatttctatgaaatatacagatggaagattaggaaataagatagatatttcgaagaactattcagtagatacagaaaagaaaaaggtagtccttcaacagatttctccatatcgtacagacttctattattctcctatagaaggatatagattcgtgacaataagatactcaaatgtttattattcctccacccaaggtttatatatcattgaaaaagattggtataaatcccaaaagaaatcaaagaagattgatgattcatttgaattctgtttctccatgcatagaaatgaaattattcgcatcaagaaagaagatgaagatatgatgtggagatttacagcaacaaataatgactcaacgaataaaattgaagttaaaccaatagggcataaaattaataagcagttaattctagcaattggaaagaagattattttattagaaaaatatgcaactgacgtatgtggaaatctatataaagtaacaaatcaggagttgaaactagaatttaaatga

SEQ ID NO: 5 - DNA, 3216 bp, synthetic construct
  source 1..3216: mol_type = other DNA
gaaaagcggaacaacggcttagtgctgggactggatatcggcgtgaccagcgtgggctacggcatcgtgaacagagaaaccggcgaggccgtggactttggtgttagactgttcagcgaatgggaccccgccaacaaccaaaacagaagagcgaagcgaagcggcagacggctgagaagcagacggaagaacagactgcaggacatgaagaagttcctggtggaccagggaatgatccctagcgattacgctttccacctggacccttacgccctgagagtgaagggactgtccgagaagctgaccgacgaggaactggccacggtgctgtataatctggcaaagctccggggcagcagcctggaaaccgtggaagataccgatgataccaagtctgatgacagcctatccaccaagaataacctgaggattaacgacgccctgataaagtctggcaagtacgtgtgtcagatccagcaggagcggctgtctgaaaaggaaaaactgcggggccacatcaataacttccggacaaaggactacgtgagcgaactgaagcagatcctgagcaatcagggcaaaagcaaggactttaacgattacgttgagaacctgatcgtgcggagaagagatttcagcgatggccccggctctcagaaaagtcctacaccttacggccggtggctgacaaaggaccaagaggaacctatcgacctgatcgagaagatgcggggaaagtgcagctactatcctgaccagctgcggtgcgccaagatggcctacaaggccgatctgttcaacctgctgaacgacctgaacaacctgagtgtgaaaggcgaaaagctggaacgcgatcagaaggaacaggtgatagaatacgtgaacgagaaaggttccatcaaacctaagcagctggccaaactgctgggcctgagcgacatcgatgacatctctggcttccggatcgataagtccgacaagccgatactgaccgagtttaagggatacatcaaggtgaagaaggtgctggaaaacagcggcctgtaccacgaccacgagctggtggaccagatcatcgatgtgctgaccagagacaaagtggtcgagcgtagggccaacgagatccggaagctgagtaacaaattctccgaggaggaaatcgaagccctggccaacctgacccagatcagcggctaccacagcatgagcgccaaagccatcaacgagttcaacaaggagatgctgatctccccactgaaccagatgcagatcataacaaacagaggaaaccagaacgacagattcaataagctgaaaggaatgaaggagatccacgccgacgacacagccatcctgagccccgtggccaagagagcccagagagaggccatgaaagtggtgaacagactacgcgagctttatggcgagttcgacagcatcgtggtcgagacaaccagagctaagaacagccaggagcagaaaaccaaaatcaaggagagccaggccttcttcgagaaagagaaaaagagagctcagcagatcgtggatgagaacaagtacaacctgaccgtcaacgccaaactcatccagaaactgcggctttacaccgagcagaattacaagaccgcctacgccctggtgcccatcgacctggacctgctgatcagcgaccctaccgccttcgaggtggaccacatcatccccatcagcatcagctacgacgacagcttttctaataaggtgctggtcacccggtgggagaatcagatcaagggaaacctgacccctctgcaagcccacaagaaccacaagttcggcgagtacagcgacttagccagctacatcgccaacgtgaacgacctgcggaaaatgaacggaatgagatacgagaagaagaagaacctgctgctgtctgacgaggatctgaccaaattcgatacccagaagaagttcatcgctagaaacatcgtggacaccgcttatgccgagagagtggtgctgaataccctgcaagattacttcagagccaacgaaatcagcacagtggtgaatacagtgaagggcaacctgacccacatgttccggaacaagatccacctggacaaggagagatctgacattgatacaggcgacgcccatcatgccatcgacgctctgatcatcgcttctctcaaagtctttcggagatttggcgaaatcctggacaagcgccccgagacaatcattgatgaagaaaccggcgaagtgctggaaatcatcgaggacaagaagttctttgaccctagatacatcgagatcgtgcacaagctgtctctgatcgagaaagatgtgaccaagttcagctacaagatcgacaccaagcccaacagacaggtggccgacgagacaatctactctacaagaaacatcaacggcgaggacctcgtggtcaagaagtacaagaacatctacgaggaaaaattcgtgacactggccaacgacatcctggaaaacaatgtggaaaagtacctgatgtacagaaatagccctgaaaccttcaagtacatcagcgacatcatcctgtattacgccaaggagtactctgatgatgaaaagctgatgaaaaacggcttgttcgtgaaaaatcccctgaacaagtactacgaggaacaccacgagtacctgcacaagcctagcaagcacaacaatggccctagtatcatcagcatgaagtatacagatggccggctgggcaacaagatcgacatcagcaagaactactctgtggacaccgagaagaagaaggtggtgctgcaacagatcagcccttaccggaccgacttctactacagccctatcgagggctaccggttcgtgaccatcagatacagcaacgtgtactacagcagcacgcagggcctgtacattatcgagaaggattggtacaagagccaaaagaaatccaaaaaaatcgacgactccttcgagttctgcttcagcatgcacaggaatgagatcatcagaatcaaaaaggaggacgaggatatgatgtggcggttcactgccacaaacaacgacagcacaaacaagatcgaggtgaagcctatcggccataagatcaacaagcagctgatcctggctattggaaagaagatcattctgctggaaaagtacgctacagatgtgtgcggcaacctgtacaaagtgaccaaccaggagctgaaactggagttcaag

SEQ ID NO: 6 - DNA, 3369 bp, synthetic construct
  source 1..3369: mol_type = other DNA
atgatccccaatcccctgctgggcctggacaagcggactgccgacggcagtgagttcgagtctcccaagaaaaagaggaaagtgggatccgaaaagcggaacaacggcttagtgctgggactggatatcggcgtgaccagcgtgggctacggcatcgtgaacagagaaaccggcgaggccgtggactttggtgttagactgttcagcgaatgggaccccgccaacaaccaaaacagaagagcgaagcgaagcggcagacggctgagaagcagacggaagaacagactgcaggacatgaagaagttcctggtggaccagggaatgatccctagcgattacgctttccacctggacccttacgccctgagagtgaagggactgtccgagaagctgaccgacgaggaactggccacggtgctgtataatctggcaaagctccggggcagcagcctggaaaccgtggaagataccgatgataccaagtctgatgacagcctatccaccaagaataacctgaggattaacgacgccctgataaagtctggcaagtacgtgtgtcagatccagcaggagcggctgtctgaaaaggaaaaactgcggggccacatcaataacttccggacaaaggactacgtgagcgaactgaagcagatcctgagcaatcagggcaaaagcaaggactttaacgattacgttgagaacctgatcgtgcggagaagagatttcagcgatggccccggctctcagaaaagtcctacaccttacggccggtggctgacaaaggaccaagaggaacctatcgacctgatcgagaagatgcggggaaagtgcagctactatcctgaccagctgcggtgcgccaagatggcctacaaggccgatctgttcaacctgctgaacgacctgaacaacctgagtgtgaaaggcgaaaagctggaacgcgatcagaaggaacaggtgatagaatacgtgaacgagaaaggttccatcaaacctaagcagctggccaaactgctgggcctgagcgacatcgatgacatctctggcttccggatcgataagtccgacaagccgatactgaccgagtttaagggatacatcaaggtgaagaaggtgctggaaaacagcggcctgtaccacgaccacgagctggtggaccagatcatcgatgtgctgaccagagacaaagtggtcgagcgtagggccaacgagatccggaagctgagtaacaaattctccgaggaggaaatcgaagccctggccaacctgacccagatcagcggctaccacagcatgagcgccaaagccatcaacgagttcaacaaggagatgctgatctccccactgaaccagatgcagatcataacaaacagaggaaaccagaacgacagattcaataagctgaaaggaatgaaggagatccacgccgacgacacagccatcctgagccccgtggccaagagagcccagagagaggccatgaaagtggtgaacagactacgcgagctttatggcgagttcgacagcatcgtggtcgagacaaccagagctaagaacagccaggagcagaaaaccaaaatcaaggagagccaggccttcttcgagaaagagaaaaagagagctcagcagatcgtggatgagaacaagtacaacctgaccgtcaacgccaaactcatccagaaactgcggctttacaccgagcagaattacaagaccgcctacgccctggtgcccatcgacctggacctgctgatcagcgaccctaccgccttcgaggtggaccacatcatccccatcagcatcagctacgacgacagcttttctaataaggtgctggtcacccggtgggagaatcagatcaagggaaacctgacccctctgcaagcccacaagaaccacaagttcggcgagtacagcgacttagccagctacatcgccaacgtgaacgacctgcggaaaatgaacggaatgagatacgagaagaagaagaacctgctgctgtctgacgaggatctgaccaaattcgatacccagaagaagttcatcgctagaaacatcgtggacaccgcttatgccgagagagtggtgctgaataccctgcaagattacttcagagccaacgaaatcagcacagtggtgaatacagtgaagggcaacctgacccacatgttccggaacaagatccacctggacaaggagagatctgacattgatacaggcgacgcccatcatgccatcgacgctctgatcatcgcttctctcaaagtctttcggagatttggcgaaatcctggacaagcgccccgagacaatcattgatgaagaaaccggcgaagtgctggaaatcatcgaggacaagaagttctttgaccctagatacatcgagatcgtgcacaagctgtctctgatcgagaaagatgtgaccaagttcagctacaagatcgacaccaagcccaacagacaggtggccgacgagacaatctactctacaagaaacatcaacggcgaggacctcgtggtcaagaagtacaagaacatctacgaggaaaaattcgtgacactggccaacgacatcctggaaaacaatgtggaaaagtacctgatgtacagaaatagccctgaaaccttcaagtacatcagcgacatcatcctgtattacgccaaggagtactctgatgatgaaaagctgatgaaaaacggcttgttcgtgaaaaatcccctgaacaagtactacgaggaacaccacgagtacctgcacaagcctagcaagcacaacaatggccctagtatcatcagcatgaagtatacagatggccggctgggcaacaagatcgacatcagcaagaactactctgtggacaccgagaagaagaaggtggtgctgcaacagatcagcccttaccggaccgacttctactacagccctatcgagggctaccggttcgtgaccatcagatacagcaacgtgtactacagcagcacgcagggcctgtacattatcgagaaggattggtacaagagccaaaagaaatccaaaaaaatcgacgactccttcgagttctgcttcagcatgcacaggaatgagatcatcagaatcaaaaaggaggacgaggatatgatgtggcggttcactgccacaaacaacgacagcacaaacaagatcgaggtgaagcctatcggccataagatcaacaagcagctgatcctggctattggaaagaagatcattctgctggaaaagtacgctacagatgtgtgcggcaacctgtacaaagtgaccaaccaggagctgaaactggagttcaagtctagaaagcggacagcagacggctccgaatttgaaagccctaagaaaaagagaaaggtgtga

SEQ ID NO: 7 - protein, 1056 aa, synthetic construct
GYILGLDLGITSVGFGIIEEDTYKIIKYGVRLFDEGTADNNLTRRQNRSSRRLKSRRKNRINAIKYLLLNNNVIPNVNFDILDNVYELRVKGLTQKLTNLELANVLVNLAKRRGSSLDVAVDENDNESVVAASALTKNTAELKKNNLYICQAQLAKIKSGVKLRNHDNIYKSDDYEKELRQILSNQDLSNELNDKIVEIIMRRRNFSEGPGSYEFPTKYGSYRVDYVDGIKTIKHVNLIEEMRGKCSIFRNEPRIAKCSFEACLFNLLNDLNNLTINGNGKITKEQKLQIINDYVIPKGKITIPQLVKYLGVEEKDITGFRKNKSEKNIITTFDTYSKMINFLPKDILNVTAYVDSIIELLTKTAVVEERIEEIKKLNLGLNDDIINQLANMTKVNGYHSLSKKAMDIIIPEMIETDKNQMQIIHDNNLDKNDCQEKGAKIKFDSSAILSPVTKRVHMQAIQVVNELRKEYGEFDSIVIETTRAKNSSDEKKDIQEDQKRLEEQKRKSDELLIDLDKNPDNYNTLTKLKLRLYKEQEGKTIYAGLPIDLDLLLNDPTAYQIEHIIPYAVSFDNSMNNKSLASAKENQDKGKRTPWQYFSSGMVSECEGPINDWESFEAFVNSLKISKNKKNNLLNQEDVTKFDNMKAFVARNLSDTSYDVRTVMNTMQQYFNDNNINTNVFTVKGRLTNIFRSRVGLKKDRDYFIHHAVDALIIAGSKNQSVFNNAYLLSSTKDGILIKKTGEVFDYSQDPFDDPRFLNYISSLKKIEGKPEDFSYKVDKKTNRQFADQTIYSTRNYDGVDYVVKKYKDIYGKDGESLKKLFDDKKEQTLLMARNDIQTFELFKKIYENYKNEKNPFAKYKDEFGPIKKYCKDGKGPEIYQVKYISDKLGNHLDISDKYNVENGNKKVVLLQISPYRTDVYQSKETGLYNFITIRRYHIYQENDMNIIKPELYQKLLSDKKIDETYEFKFSLNRNDIINLVTIDNQADHIDEMLYRFIGTNNDKTNKIEIRYIEKMTLKQIMVSIGKKIVKFEKYNVSPTGKKAKAEKEVLKLKWK

SEQ ID NO: 8 - protein, 1057 aa, synthetic construct
MGYILGLDLGITSVGFGIIEEDTYKIIKYGVRLFDEGTADNNLTRRQNRSSRRLKSRRKNRINAIKYLLLNNNVIPNVNFDILDNVYELRVKGLTQKLTNLELANVLVNLAKRRGSSLDVAVDENDNESVVAASALTKNTAELKKNNLYICQAQLAKIKSGVKLRNHDNIYKSDDYEKELRQILSNQDLSNELNDKIVEIIMRRRNFSEGPGSYEFPTKYGSYRVDYVDGIKTIKHVNLIEEMRGKCSIFRNEPRIAKCSFEACLFNLLNDLNNLTINGNGKITKEQKLQIINDYVIPKGKITIPQLVKYLGVEEKDITGFRKNKSEKNIITTFDTYSKMINFLPKDILNVTAYVDSIIELLTKTAVVEERIEEIKKLNLGLNDDIINQLANMTKVNGYHSLSKKAMDIIIPEMIETDKNQMQIIHDNNLDKNDCQEKGAKIKFDSSAILSPVTKRVHMQAIQVVNELRKEYGEFDSIVIETTRAKNSSDEKKDIQEDQKRLEEQKRKSDELLIDLDKNPDNYNTLTKLKLRLYKEQEGKTIYAGLPIDLDLLLNDPTAYQIEHIIPYAVSFDNSMNNKSLASAKENQDKGKRTPWQYFSSGMVSECEGPINDWESFEAFVNSLKISKNKKNNLLNQEDVTKFDNMKAFVARNLSDTSYDVRTVMNTMQQYFNDNNINTNVFTVKGRLTNIFRSRVGLKKDRDYFIHHAVDALIIAGSKNQSVFNNAYLLSSTKDGILIKKTGEVFDYSQDPFDDPRFLNYISSLKKIEGKPEDFSYKVDKKTNRQFADQTIYSTRNYDGVDYVVKKYKDIYGKDGESLKKLFDDKKEQTLLMARNDIQTFELFKKIYENYKNEKNPFAKYKDEFGPIKKYCKDGKGPEIYQVKYISDKLGNHLDISDKYNVENGNKKVVLLQISPYRTDVYQSKETGLYNFITIRRYHIYQENDMNIIKPELYQKLLSDKKIDETYEFKFSLNRNDIINLVTIDNQADHIDEMLYRFIGTNNDKTNKIEIRYIEKMTLKQIMVSIGKKIVKFEKYNVSPTGKKAKAEKEVLKLKWK

SEQ ID NO: 9 - protein, 1106 aa, synthetic construct
MIPNPLLGLDKRTADGSEFESPKKKRKVGSGYILGLDLGITSVGFGIIEEDTYKIIKYGVRLFDEGTADNNLTRRQNRSSRRLKSRRKNRINAIKYLLLNNNVIPNVNFDILDNVYELRVKGLTQKLTNLELANVLVNLAKRRGSSLDVAVDENDNESVVAASALTKNTAELKKNNLYICQAQLAKIKSGVKLRNHDNIYKSDDYEKELRQILSNQDLSNELNDKIVEIIMRRRNFSEGPGSYEFPTKYGSYRVDYVDGIKTIKHVNLIEEMRGKCSIFRNEPRIAKCSFEACLFNLLNDLNNLTINGNGKITKEQKLQIINDYVIPKGKITIPQLVKYLGVEEKDITGFRKNKSEKNIITTFDTYSKMINFLPKDILNVTAYVDSIIELLTKTAVVEERIEEIKKLNLGLNDDIINQLANMTKVNGYHSLSKKAMDIIIPEMIETDKNQMQIIHDNNLDKNDCQEKGAKIKFDSSAILSPVTKRVHMQAIQVVNELRKEYGEFDSIVIETTRAKNSSDEKKDIQEDQKRLEEQKRKSDELLIDLDKNPDNYNTLTKLKLRLYKEQEGKTIYAGLPIDLDLLLNDPTAYQIEHIIPYAVSFDNSMNNKSLASAKENQDKGKRTPWQYFSSGMVSECEGPINDWESFEAFVNSLKISKNKKNNLLNQEDVTKFDNMKAFVARNLSDTSYDVRTVMNTMQQYFNDNNINTNVFTVKGRLTNIFRSRVGLKKDRDYFIHHAVDALIIAGSKNQSVFNNAYLLSSTKDGILIKKTGEVFDYSQDPFDDPRFLNYISSLKKIEGKPEDFSYKVDKKTNRQFADQTIYSTRNYDGVDYVVKKYKDIYGKDGESLKKLFDDKKEQTLLMARNDIQTFELFKKIYENYKNEKNPFAKYKDEFGPIKKYCKDGKGPEIYQVKYISDKLGNHLDISDKYNVENGNKKVVLLQISPYRTDVYQSKETGLYNFITIRRYHIYQENDMNIIKPELYQKLLSDKKIDETYEFKFSLNRNDIINLVTIDNQADHIDEMLYRFIGTNNDKTNKIEIRYIEKMTLKQIMVSIGKKIVKFEKYNVSPTGKKAKAEKEVLKLKWKSRKRTADGSEFESPKKKRKV

SEQ ID NO: 10 - DNA, 3174 bp, synthetic construct
  source 1..3174: mol_type = other DNA
atgggatatattttaggattagatttaggaattactagtgtaggctttggtattattgaggaggatacatataaaataataaaatatggtgttagattatttgatgaaggtacagcggataataatttaacaagaagacaaaatcgttcatcaagaagacttaagagtagacgtaaaaatagaataaatgcgattaaatatttattattaaataataatgttataccgaatgttaattttgatattttagacaacgtatatgaattaagagtaaaaggcctaactcaaaaattgactaatctagagcttgctaatgtattagttaatttagctaaaagacgtggatcatcattagatgttgctgtagatgaaaacgataatgaaagtgttgtagcagcgtcagcattaactaaaaatactgcagaactaaagaaaaataatttatacatttgtcaggctcaattagctaaaattaaaagtggagtaaagcttagaaaccacgataatatttataaaagtgatgattacgaaaaagaacttagacaaattctttctaatcaagatttatctaatgaactaaatgataaaatagttgaaattattatgcgaagaagaaatttctcggaaggaccaggttcttatgaatttccaactaaatatggttcatatcgtgttgattatgttgacggcattaagacgattaaacatgtaaacttaattgaagaaatgcgtggcaagtgttctattttcagaaatgagccacgtattgcaaaatgtagttttgaggcttgcttatttaatttattaaatgatttaaataatcttacaattaacggtaatggaaaaataacaaaagagcaaaaattacaaataattaatgattatgttattccaaagggaaaaataacaattccacaattagttaaatatttaggagttgaggaaaaggatattacaggatttagaaaaaataagtctgagaaaaatataattacaacatttgatacatattctaagatgattaattttttacctaaggatattcttaatgtaacagcatatgtagattcaattattgaattacttactaaaactgctgtagttgaagagagaattgaagaaattaagaaattaaaccttggattaaacgacgatattattaatcaacttgcaaatatgaccaaggttaatggatatcattcactaagtaaaaaagcaatggatattattattcctgagatgattgaaactgataaaaatcaaatgcaaattattcatgataataatttagataagaatgattgtcaagaaaaaggggctaaaataaagtttgattcatcagcaattttatcaccagttacaaagcgtgttcatatgcaggcaattcaggttgtcaacgaacttagaaaggaatatggtgaatttgattcaattgttattgaaacaacaagagctaaaaacagtagtgatgaaaagaaagatatccaagaggatcaaaaaagattagaagaacaaaaaagaaaatctgatgaacttcttatagatcttgataagaatcctgataattataatactttaactaagcttaagttaagattatataaggaacaagaaggaaaaacaatttatgctggtttaccaattgaccttgatttattgcttaatgatcctactgcttatcaaattgagcatattattccttatgctgtatcttttgataatagtatgaataataagagtcttgcatcagcaaaagaaaatcaagataaaggaaaacgaacaccttggcagtatttttcatctggtatggtttcagaatgcgaaggaccaattaatgattgggagtcctttgaagcatttgttaatagtttaaagatatcaaaaaataaaaaaaataatcttttaaatcaagaagatgtaactaaatttgataatatgaaggcgtttgttgcaagaaatttaagtgatactagttatgatgttagaactgttatgaatacgatgcaacaatattttaatgataataatattaatacaaatgtatttacagtcaagggaagattaaccaatattttcagaagcagagtagggcttaaaaaggatagagattattttattcatcatgcagtagatgcattaatcattgctggttctaaaaatcagtcagttttcaataatgcatatttattatcatcgacaaaagatggaatacttattaaaaaaacaggagaggtatttgattattctcaagatccgtttgatgatcctaggtttttaaattatatttcatcacttaaaaaaattgaaggaaagcctgaagatttttcatataaggttgataagaagacaaatagacaatttgcagatcaaacaatttattcaacaagaaattatgatggtgttgattacgttgtaaagaaatataaagatatttatggtaaagatggagaatcattaaagaagttatttgatgataaaaaagaacaaacccttttaatggctaggaacgatattcaaacatttgaactatttaagaaaatatatgaaaactataagaatgaaaaaaatccattcgctaaatataaggatgaatttggaccaataaagaaatattgtaaagatggaaaaggacctgaaatttaccaggttaaatatatttctgacaaattaggtaatcatttagatatttcggataaatataacgtagaaaatggtaataaaaaggttgttttacttcaaatttcaccatatagaacggatgtgtatcaatctaaagaaactggactttataattttattacaataaggagatatcatatttatcaggaaaatgatatgaatattataaagcctgaattgtatcaaaaattattgtcggataaaaaaatcgatgaaacttatgaatttaaattttcattaaatagaaacgatattattaatttagttacaatagataatcaagctgatcatattgatgagatgctctatagatttattggaactaataatgataagacaaataaaattgagattagatatattgaaaaaatgacattaaaacaaattatggtttcaattggtaagaaaattgttaaatttgaaaaatataatgttagtccaacaggtaaaaaagccaaagctgaaaaagaagtattgaaattaaagtggaagtag

SEQ ID NO: 11 - DNA, 3168 bp, synthetic construct
  source 1..3168: mol_type = other DNA
ggctacatcctgggcctggacctgggcatcaccagcgtgggatttggcatcatcgaagaggatacctacaaaatcatcaaatatggcgtcagactgtttgatgagggaacagccgacaataacctgaccagacggcagaacagaagcagccgtagactgaagagcagacggaagaatagaatcaacgcaatcaagtacctcctccttaataacaatgtgatccctaacgtgaactttgatatcctggacaacgtgtacgaactgagagtgaagggcctgacccagaagctgaccaacctggaactggccaacgtcctggtgaacctcgctaaaagaaggggctctagcctcgacgttgccgtggacgagaacgacaacgagagcgtggtggccgccagcgccctgaccaagaacaccgctgaactgaagaagaacaacctgtacatttgtcaggcccagctggccaagataaagagcggcgtgaaactgagaaaccatgataacatctacaagtctgacgactacgagaaggaactgagacagatcctgtctaaccaagacctgtccaacgaactgaacgacaagatcgtggaaatcatcatgagaagaagaaatttcagcgagggccccggctcttacgagttccctaccaagtacggcagctacagagtggattacgtggacggcatcaagaccatcaagcacgtgaatctgatcgaggaaatgcggggcaaatgcagcatcttcagaaacgagcctagaatcgctaagtgcagttttgaggcctgcctgttcaatttgctgaacgatctgaacaacctgacaattaacggcaacggcaagatcacaaaggaacagaagctgcagatcattaacgattatgtgatccctaagggaaagatcaccataccccagctggtgaagtatctgggcgtggaagagaaagacatcaccggtttcagaaagaacaaatcagagaagaacatcatcacaacattcgatacatacagcaagatgataaatttcctgccaaaggacatcctcaacgtgaccgcctacgttgattccatcatcgagctgctgaccaaaaccgccgtggttgaggaaagaatcgaagagatcaagaaactgaatctcggactgaacgacgacatcatcaaccagctggctaatatgaccaaggtgaacggctaccactctctgagcaagaaggctatggacatcatcatccctgagatgatcgagacagataaaaaccagatgcaaatcatccacgacaacaacctggacaagaacgactgccaggagaagggcgccaagatcaagttcgacagcagcgccatcctgtctcctgtgaccaaaagggtgcacatgcaggccatccaggtggtgaacgagctccgcaaggaatacggagagttcgacagcatcgtgatcgaaaccacccgggccaagaatagctccgacgagaagaaggatattcaggaggatcagaagcggctggaagagcagaaaagaaagagcgacgaactgctgatcgacctggacaaaaaccccgataattacaacaccctgacaaagctgaaactgagactgtacaaggagcaggagggcaagaccatctacgccggcctgcctatcgacctggatctactgctgaatgatcctaccgcctaccagatcgagcacatcatcccctacgccgtgtcttttgacaacagcatgaacaacaagtccctggccagcgccaaggagaaccaagacaagggaaagagaacaccttggcagtacttctctagcggcatggtcagcgagtgcgagggccctatcaacgactgggaatctttcgaggccttcgtgaacagcctgaagatatctaagaacaagaagaacaacctgctgaatcaggaggacgtcacaaagtttgataatatgaaggctttcgtggcccggaacctgagcgacacctcttacgacgtgcggaccgttatgaacaccatgcaacagtactttaacgataacaatatcaataccaatgtgttcaccgtgaaaggcagactgacaaacatattccgaagccgggtgggcctgaaaaaggacagagattatttcatccaccacgccgtggatgccctgatcatcgccggaagcaagaatcagtctgtgttcaacaacgcctacctgctgagctctaccaaggacggcatcctgatcaagaaaaccggcgaggtgttcgactactcacaggaccccttcgacgaccccagattcctgaactacatcagctccctgaaaaagatcgaaggaaagcccgaggatttcagctacaaggtggataagaaaaccaatcggcaatttgccgaccagaccatctacagcacccggaattacgacggcgtggactacgtggtgaagaaatacaaggatatctacggcaaggacggcgagagcctgaaaaagctgttcgacgacaagaaagaacagaccctgctgatggccagaaacgacatccagaccttcgagctgttcaagaagatctacgagaactacaagaacgagaagaatcccttcgccaagtataaggatgagttcggcccaatcaagaagtactgcaaggacggcaagggccctgagatctaccaagtgaagtatatcagcgacaagctgggaaaccacctggacatcagcgacaagtacaacgtggaaaacggcaataagaaggtggtcctgctccagatctccccttatcggacagacgtgtatcagagcaaggaaaccggactgtacaacttcatcactattcggcgctaccacatctaccaggagaacgacatgaacattattaaacctgaactgtaccaaaagctgctgtctgacaagaagatcgacgagacatacgagttcaagttcagcctcaatagaaacgatatcattaacctggtgaccattgacaaccaggcggaccacatcgatgaaatgctgtacagattcatcggcacaaacaacgacaaaacaaacaagatcgagatccggtacatcgaaaagatgacactgaaacagatcatggtgtccatcggcaagaagatcgtcaaattcgaaaagtacaacgtgtccccaacaggcaagaaggccaaggccgagaaggaagtgctgaagctgaaatggaag

SEQ ID NO: 12 - DNA, 3321 bp, synthetic construct
  source 1..3321: mol_type = other DNA
atgatccccaatcccctgctgggcctggacaagcggactgccgacggcagtgagttcgagtctcccaagaaaaagaggaaagtgggatccggctacatcctgggcctggacctgggcatcaccagcgtgggatttggcatcatcgaagaggatacctacaaaatcatcaaatatggcgtcagactgtttgatgagggaacagccgacaataacctgaccagacggcagaacagaagcagccgtagactgaagagcagacggaagaatagaatcaacgcaatcaagtacctcctccttaataacaatgtgatccctaacgtgaactttgatatcctggacaacgtgtacgaactgagagtgaagggcctgacccagaagctgaccaacctggaactggccaacgtcctggtgaacctcgctaaaagaaggggctctagcctcgacgttgccgtggacgagaacgacaacgagagcgtggtggccgccagcgccctgaccaagaacaccgctgaactgaagaagaacaacctgtacatttgtcaggcccagctggccaagataaagagcggcgtgaaactgagaaaccatgataacatctacaagtctgacgactacgagaaggaactgagacagatcctgtctaaccaagacctgtccaacgaactgaacgacaagatcgtggaaatcatcatgagaagaagaaatttcagcgagggccccggctcttacgagttccctaccaagtacggcagctacagagtggattacgtggacggcatcaagaccatcaagcacgtgaatctgatcgaggaaatgcggggcaaatgcagcatcttcagaaacgagcctagaatcgctaagtgcagttttgaggcctgcctgttcaatttgctgaacgatctgaacaacctgacaattaacggcaacggcaagatcacaaaggaacagaagctgcagatcattaacgattatgtgatccctaagggaaagatcaccataccccagctggtgaagtatctgggcgtggaagagaaagacatcaccggtttcagaaagaacaaatcagagaagaacatcatcacaacattcgatacatacagcaagatgataaatttcctgccaaaggacatcctcaacgtgaccgcctacgttgattccatcatcgagctgctgaccaaaaccgccgtggttgaggaaagaatcgaagagatcaagaaactgaatctcggactgaacgacgacatcatcaaccagctggctaatatgaccaaggtgaacggctaccactctctgagcaagaaggctatggacatcatcatccctgagatgatcgagacagataaaaaccagatgcaaatcatccacgacaacaacctggacaagaacgactgccaggagaagggcgccaagatcaagttcgacagcagcgccatcctgtctcctgtgaccaaaagggtgcacatgcaggccatccaggtggtgaacgagctccgcaaggaatacggagagttcgacagcatcgtgatcgaaaccacccgggccaagaatagctccgacgagaagaaggatattcaggaggatcagaagcggctggaagagcagaaaagaaagagcgacgaactgctgatcgacctggacaaaaaccccgataattacaacaccctgacaaagctgaaactgagactgtacaaggagcaggagggcaagaccatctacgccggcctgcctatcgacctggatctactgctgaatgatcctaccgcctaccagatcgagcacatcatcccctacgccgtgtcttttgacaacagcatgaacaacaagtccctggccagcgccaaggagaaccaagacaagggaaagagaacaccttggcagtacttctctagcggcatggtcagcgagtgcgagggccctatcaacgactgggaatctttcgaggccttcgtgaacagcctgaagatatctaagaacaagaagaacaacctgctgaatcaggaggacgtcacaaagtttgataatatgaaggctttcgtggcccggaacctgagcgacacctcttacgacgtgcggaccgttatgaacaccatgcaacagtactttaacgataacaatatcaataccaatgtgttcaccgtgaaaggcagactgacaaacatattccgaagccgggtgggcctgaaaaaggacagagattatttcatccaccacgccgtggatgccctgatcatcgccggaagcaagaatcagtctgtgttcaacaacgcctacctgctgagctctaccaaggacggcatcctgatcaagaaaaccggcgaggtgttcgactactcacaggaccccttcgacgaccccagattcctgaactacatcagctccctgaaaaagatcgaaggaaagcccgaggatttcagctacaaggtggataagaaaaccaatcggcaatttgccgaccagaccatctacagcacccggaattacgacggcgtggactacgtggtgaagaaatacaaggatatctacggcaaggacggcgagagcctgaaaaagctgttcgacgacaagaaagaacagaccctgctgatggccagaaacgacatccagaccttcgagctgttcaagaagatctacgagaactacaagaacgagaagaatcccttcgccaagtataaggatgagttcggcccaatcaagaagtactgcaaggacggcaagggccctgagatctaccaagtgaagtatatcagcgacaagctgggaaaccacctggacatcagcgacaagtacaacgtggaaaacggcaataagaaggtggtcctgctccagatctccccttatcggacagacgtgtatcagagcaaggaaaccggactgtacaacttcatcactattcggcgctaccacatctaccaggagaacgacatgaacattattaaacctgaactgtaccaaaagctgctgtctgacaagaagatcgacgagacatacgagttcaagttcagcctcaatagaaacgatatcattaacctggtgaccattgacaaccaggcggaccacatcgatgaaatgctgtacagattcatcggcacaaacaacgacaaaacaaacaagatcgagatccggtacatcgaaaagatgacactgaaacagatcatggtgtccatcggcaagaagatcgtcaaattcgaaaagtacaacgtgtccccaacaggcaagaaggccaaggccgagaaggaagtgctgaagctgaaatggaagtctagaaagcggacagcagacggctccgaatttgaaagccctaagaaaaagagaaaggtgtga

SEQ ID NO: 13 - protein, 1070 aa, synthetic construct
AKYILGLDMGITSVGWGLIDYDSGKIVDKGVRLFKEATAEDNLNRRTHRGSRRLKRRRQQRIIELKRLLKKENILTDDYTPLNNVYYLRCKGLNEKLTNAELVSVLLHIAKIRGSSLEVVEDDEKKANEDKSSKANLSANEVKLRTSKEEICEKQYKDLIENGKVRGINNIYKSSSYESELKKILANQGLSKEVNDKIINIIFRRRDFNEGPGSMESPTIYGRFIPHENGEVEVIHSMIEKMQGKCSIYQNEPRAAKMSFTADLFNFLNDLNNLEIQRADNYDNYITPDEKKLIINEYIIKKGKITIQKLCDVLQVKKENITGFRVNKGEPILTEFKGYQKVLKALNRIIDTEIVDAVIDVLTMHKDVEKRKEEIKKINNTEFSEEDIVKLSEITGIQQYHSLSYKAMKVIIPELYENPENQMEVITRLNLVKTSIDDLKGRTTIPSYSNRIYSPVAKRVHNETIKVINAVIKKYGDLDSIVIEMARDRNTAEERKNIEQAQKRNQENREKAKEILNVNYSFSKLDSKLVQKIMLYNQQDGKSMYSGKPINLNVLISDPNAYEIDHIIPLSISLDDSIANKVLVYRSENQQKLNKTPLQYLRSGNSNGWSVDEFREVVIKMYNDKKISLKKLQNLLCEKDITKQDVRKEFIERNLVDTRYASRVVLGLLKDYFKANNKNTKVFTIRGQITSQFRKSRNLVKSRDCFYHHIVDALIIAYFRKFNYINELMQYNFNYKGKIDEETGELIQYNDTLFSTKDDDFTIQLRKLNDLNKYSEDIKISHKVDRKPNRKFSDETIYSAIKIGNDYMKISKYKDIYGEEGKKFAKKIKDNLQKENELDIFLMKKVDPETFNLLVSIVKNTICGEKENPFVKYCEENNIDFIRKKSKNGNGPIIRSIRYYDGKVNSYQDISQKYSNLSDDKKVVRLHISPYRTDFYLTKEGNYKFLTIKYANIKENHNGYYIPKEVYETLKKSKKINDSDEFLFSLYRNDYLRITTNDPTENAPMIYRFVGTKKDLNNVIEVKPIYCDKIDKPQIEKTIGKKVTKIEKFDVDVLGNKIAKKRDKCLQLEL

SEQ ID NO: 14 - protein, 1071 aa, synthetic construct
VAKYILGLDMGITSVGWGLIDYDSGKIVDKGVRLFKEATAEDNLNRRTHRGSRRLKRRRQQRIIELKRLLKKENILTDDYTPLNNVYYLRCKGLNEKLTNAELVSVLLHIAKIRGSSLEVVEDDEKKANEDKSSKANLSANEVKLRTSKEEICEKQYKDLIENGKVRGINNIYKSSSYESELKKILANQGLSKEVNDKIINIIFRRRDFNEGPGSMESPTIYGRFIPHENGEVEVIHSMIEKMQGKCSIYQNEPRAAKMSFTADLFNFLNDLNNLEIQRADNYDNYITPDEKKLIINEYIIKKGKITIQKLCDVLQVKKENITGFRVNKGEPILTEFKGYQKVLKALNRIIDTEIVDAVIDVLTMHKDVEKRKEEIKKINNTEFSEEDIVKLSEITGIQQYHSLSYKAMKVIIPELYENPENQMEVITRLNLVKTSIDDLKGRTTIPSYSNRIYSPVAKRVHNETIKVINAVIKKYGDLDSIVIEMARDRNTAEERKNIEQAQKRNQENREKAKEILNVNYSFSKLDSKLVQKIMLYNQQDGKSMYSGKPINLNVLISDPNAYEIDHIIPLSISLDDSIANKVLVYRSENQQKLNKTPLQYLRSGNSNGWSVDEFREVVIKMYNDKKISLKKLQNLLCEKDITKQDVRKEFIERNLVDTRYASRVVLGLLKDYFKANNKNTKVFTIRGQITSQFRKSRNLVKSRDCFYHHIVDALIIAYFRKFNYINELMQYNFNYKGKIDEETGELIQYNDTLFSTKDDDFTIQLRKLNDLNKYSEDIKISHKVDRKPNRKFSDETIYSAIKIGNDYMKISKYKDIYGEEGKKFAKKIKDNLQKENELDIFLMKKVDPETFNLLVSIVKNTICGEKENPFVKYCEENNIDFIRKKSKNGNGPIIRSIRYYDGKVNSYQDISQKYSNLSDDKKVVRLHISPYRTDFYLTKEGNYKFLTIKYANIKENHNGYYIPKEVYETLKKSKKINDSDEFLFSLYRNDYLRITTNDPTENAPMIYRFVGTKKDLNNVIEVKPIYCDKIDKPQIEKTIGKKVTKIEKFDVDVLGNKIAKKRDKCLQLEL

SEQ ID NO: 15 - protein, 1120 aa, synthetic construct
MIPNPLLGLDKRTADGSEFESPKKKRKVGSAKYILGLDMGITSVGWGLIDYDSGKIVDKGVRLFKEATAEDNLNRRTHRGSRRLKRRRQQRIIELKRLLKKENILTDDYTPLNNVYYLRCKGLNEKLTNAELVSVLLHIAKIRGSSLEVVEDDEKKANEDKSSKANLSANEVKLRTSKEEICEKQYKDLIENGKVRGINNIYKSSSYESELKKILANQGLSKEVNDKIINIIFRRRDFNEGPGSMESPTIYGRFIPHENGEVEVIHSMIEKMQGKCSIYQNEPRAAKMSFTADLFNFLNDLNNLEIQRADNYDNYITPDEKKLIINEYIIKKGKITIQKLCDVLQVKKENITGFRVNKGEPILTEFKGYQKVLKALNRIIDTEIVDAVIDVLTMHKDVEKRKEEIKKINNTEFSEEDIVKLSEITGIQQYHSLSYKAMKVIIPELYENPENQMEVITRLNLVKTSIDDLKGRTTIPSYSNRIYSPVAKRVHNETIKVINAVIKKYGDLDSIVIEMARDRNTAEERKNIEQAQKRNQENREKAKEILNVNYSFSKLDSKLVQKIMLYNQQDGKSMYSGKPINLNVLISDPNAYEIDHIIPLSISLDDSIANKVLVYRSENQQKLNKTPLQYLRSGNSNGWSVDEFREVVIKMYNDKKISLKKLQNLLCEKDITKQDVRKEFIERNLVDTRYASRVVLGLLKDYFKANNKNTKVFTIRGQITSQFRKSRNLVKSRDCFYHHIVDALIIAYFRKFNYINELMQYNFNYKGKIDEETGELIQYNDTLFSTKDDDFTIQLRKLNDLNKYSEDIKISHKVDRKPNRKFSDETIYSAIKIGNDYMKISKYKDIYGEEGKKFAKKIKDNLQKENELDIFLMKKVDPETFNLLVSIVKNTICGEKENPFVKYCEENNIDFIRKKSKNGNGPIIRSIRYYDGKVNSYQDISQKYSNLSDDKKVVRLHISPYRTDFYLTKEGNYKFLTIKYANIKENHNGYYIPKEVYETLKKSKKINDSDEFLFSLYRNDYLRITTNDPTENAPMIYRFVGTKKDLNNVIEVKPIYCDKIDKPQIEKTIGKKVTKIEKFDVDVLGNKIAKKRDKCLQLELSRKRTADGSEFESPKKKRKV

SEQ ID NO: 16 - DNA, 3216 bp, synthetic construct
  source 1..3216: mol_type = other DNA
gtggcaaaatatatattaggactagatatgggaattacttcggtaggttggggcctaattgactatgattctggaaaaatagttgataaaggcgttagactttttaaagaagcaactgcggaagataatttaaacagacgtactcatcgtggttcaagaaggttaaaaagacgtagacaacaaagaataattgaattaaaacgattattaaaaaaagaaaacattcttacagatgattatactcctttaaataatgtttactatcttagatgtaaaggcttaaacgaaaaattaacaaatgctgaacttgtatcagtattattacatatagcaaaaataagaggaagttccctagaagtagttgaagatgatgaaaaaaaggctaatgaggataaaagtagtaaagctaatttatcagcaaacgaagtaaaattaagaacttcaaaagaagaaatttgtgaaaaacaatataaagatctaatcgaaaatggtaaagttcgtggaataaataatatttataaaagttcttcatatgagtcagaactcaaaaaaattttagctaatcaaggcttgtcaaaagaagttaatgataagataataaatataattttcagaagacgtgactttaatgaaggaccaggaagcatggaatcgcctactatatatggtagatttataccacatgaaaatggtgaggtagaagttatacatagtatgattgaaaaaatgcaagggaaatgttccatataccaaaacgaacctcgtgcagcaaaaatgtcatttacagctgatttgtttaattttttaaatgatttaaacaatttagaaatacagcgtgctgataactatgataattatattaccccagacgaaaaaaaactaataataaatgagtatatcataaaaaaaggtaaaattacaattcaaaaattatgcgatgtactacaggtaaaaaaagaaaatattactggctttagagttaacaaaggtgaaccaatactcactgaatttaaaggttatcaaaaagtattaaaagctttaaatagaataattgatactgaaattgttgatgcggttattgatgttttaacaatgcataaagatgtagaaaaaagaaaagaagaaattaaaaaaataaacaatacagagtttagcgaagaagacattgtaaaattatctgaaataaccggtatacaacagtatcattcattgtcttataaagcaatgaaagtaataatacctgaattatatgaaaatcctgaaaatcaaatggaagtaattacaagattaaatctagtgaaaacttcaatagatgatttgaaaggaagaacaacaataccttcatatagcaacagaatatattcccctgtagctaaacgtgtacataatgagacaatcaaagtaataaatgcggtaataaaaaaatatggagatttagatagtattgttatagaaatggcaagggatagaaatactgccgaagaaagaaaaaatattgaacaagcccaaaaacgaaatcaagaaaatagagaaaaagcaaaagagatactaaatgttaattatagtttttcgaagcttgattcaaaattagttcaaaagatcatgctttataatcaacaagatggtaaaagtatgtatagtggaaaaccaataaacttaaatgttttgataagtgaccctaatgcatacgagatagatcatatcataccactatcaatttcgcttgatgattcaattgccaataaagttctggtgtatagaagtgaaaaccaacaaaaattaaataaaacaccacttcaatacttacgtagtggtaattcaaatggttggagcgtcgacgaattccgtgaagttgtaataaaaatgtataatgataaaaaaatatcactaaaaaaattacaaaacttattatgtgaaaaagatataacaaaacaagatgtaagaaaagaatttattgaaagaaatttagttgacacaagatatgcaagtagggtagttttgggacttctaaaagattatttcaaagccaacaataaaaatactaaagtatttacaataagaggccaaataacatcacaattcagaaaaagtagaaacttggtgaaaagtagagattgcttttatcatcatatagtagatgcattaataattgcatattttagaaaattcaattatattaatgaattaatgcaatacaattttaattataaagggaaaattgatgaagaaacaggagaattaatccaatataatgatacgttgttttcaacgaaggatgatgatttcacaatacaattaagaaaactaaatgaccttaataaatatagtgaagatatcaaaatttcgcataaagttgatagaaagcctaatagaaagtttagtgatgaaacaatctattctgcgataaagatcggcaacgactatatgaaaatttcgaaatataaagatatttacggtgaagaaggcaaaaaatttgcaaaaaaaattaaagataatcttcaaaaggaaaatgaattagatatattcttaatgaaaaaagttgatccagaaactttcaatttattagttagtattgttaagaatacaatatgtggtgaaaaagaaaatccatttgttaaatactgtgaagaaaacaatatagattttattagaaaaaaatctaaaaatggcaatggtccgattattaggtctattagatattatgatggtaaagtaaatagttaccaggatatctcacaaaaatattcaaacttatcagatgacaaaaaagtggtgcgtttacacatttcgccgtatagaactgatttttatttaactaaagaaggaaactataaattcctaacaattaaatatgctaatatcaaagaaaaccataatggatattatataccaaaagaagtttacgaaacattaaaaaaatcaaagaaaataaatgatagtgatgaatttttattttcgctatatcgaaatgattacctaagaattacaacaaatgatcctactgaaaatgctcctatgatttatcgatttgtaggaacaaaaaaagatctaaataacgttatagaagtaaaaccaatttattgtgataagatagataaaccacaaattgagaaaaccataggtaaaaaagtaactaaaattgaaaagtttgatgttgatgtactaggtaataaaattgctaaaaaaagagataaatgcttgcaattagagctatag

SEQ ID NO: 17 - DNA, 3210 bp, synthetic construct
  source 1..3210: mol_type = other DNA
gccaagtacatcctgggactggatatgggcatcaccagcgtgggctggggcctgatcgactacgacagcggaaagatcgtggataagggcgtccggctgtttaaagaggcaaccgccgaggataatctgaacagaaggacacacagaggcagccggcggctgaagcgcagacggcagcaaagaatcatcgagctgaaaagactgctgaagaaggaaaacatcctgacagatgactacacccctctgaataatgtgtactacctgagatgcaaaggcctgaacgagaagctgaccaacgctgaactggtgtctgtgctgctgcacatcgccaagatacggggcagcagccttgaggtcgtggaagatgacgagaagaaggcgaatgaggacaagagctccaaggccaatctgagcgctaacgaggtgaagctcagaacatctaaggaggagatctgcgagaagcagtacaaggacctaatagagaacggaaaggtccggggcatcaataacatctataagtctagctcctacgaatcagagctgaagaaaatcctggccaatcagggcctgagcaaagaggtgaacgacaagatcatcaacatcattttccgccggcgagacttcaacgaaggccccgggagcatggaaagccctacaatctacggcagattcatccctcacgagaacggcgaggttgaagtgatccacagcatgatcgaaaagatgcagggcaaatgcagcatctaccagaacgagcctagagccgctaagatgagcttcacagccgacctgttcaatttcctgaacgacctgaacaacctggaaatccagagagccgataactacgacaactacattacccctgatgagaaaaagctgatcatcaacgagtacatcatcaagaaaggcaagatcaccattcagaaactctgcgacgtgctgcaagtgaagaaggaaaatatcaccggcttccgggtgaacaagggcgaacctatcctgacagagtttaagggctaccaaaaggtgctgaaggccctgaaccggatcatcgacaccgagatcgtggacgccgtgatcgacgtgctgaccatgcacaaagacgtggaaaagagaaaggaggagatcaaaaagatcaacaacaccgagttttctgaagaggatatcgtgaagctgtctgaaatcactggaatccagcagtaccattctctgagctacaaagccatgaaagtgatcatccctgagctgtacgagaacccagagaaccagatggaagtgatcaccagactgaacctggttaagacaagcatcgacgacctgaagggaagaaccaccattccatcttacagcaatagaatctatagccccgtggccaagagggtccataatgaaactattaaggtgatcaacgccgtgatcaagaaatacggcgacctggattccattgtgatcgaaatggccagagatagaaacaccgccgaagagcggaagaacatcgaacaggcccagaagcggaatcaagagaatagggaaaaggctaaagaaatcctgaacgtgaactacagctttagcaagctggacagcaagctggtccagaaaattatgctgtacaaccagcaggacggaaagagcatgtacagcggcaagcccatcaacctgaacgtgctgatctccgatcccaatgcttacgagatcgaccacatcatccccttgtccattagtctggatgatagcatcgccaacaaagtgctggtgtacagaagcgagaaccagcagaagctgaacaagacccctctgcagtacctgagatctggcaatagcaacggctggagcgtggacgagttcagagaggtggtgatcaagatgtacaacgacaaaaaaatctctctgaagaagctgcagaacctgctctgtgaaaaggacattaccaagcaggacgtgcggaaagagttcatcgaacggaacctggtggacacccggtacgccagcagagtggtgctgggcctgctgaaggactacttcaaggccaacaacaagaacaccaaggtctttaccatcagaggccagatcaccagccagttcagaaagagcagaaacctggtgaaatctagggattgtttctaccaccacatcgtggacgccctgatcatcgcctacttcagaaagtttaactatatcaatgaactgatgcagtacaacttcaactacaagggaaagattgacgaggagacaggcgagctgatccagtacaatgacaccctgttcagcaccaaggacgatgactttaccatccaactgagaaagctgaacgacctgaacaagtacagcgaggatatcaagatcagccacaaggtggatagaaagcctaaccggaaattctccgacgagacaatctacagcgccatcaaaatcggaaacgactacatgaagatatccaagtataaggatatctatggagaggaaggcaaaaagttcgccaagaaaatcaaggacaacctgcaaaaagaaaatgagctggatatcttcctgatgaagaaagtggaccccgagacattcaacttactagtgagcatcgtgaagaacacaatctgcggcgagaaggagaaccccttcgtaaaatattgcgaggaaaataatatcgacttcatcagaaaaaagtctaagaacggcaatggccctatcatccggtccatcagatactacgatggcaaagttaacagctaccaggacatcagccagaagtacagcaacctgagcgatgacaagaaggttgtgcggctgcacatctctccttaccggaccgacttctacctgaccaaggaaggcaactacaagttcctgaccatcaagtacgccaacatcaaggagaaccacaacggctactatatccctaaggaagtgtacgagacactgaagaagtctaagaagatcaacgacagcgacgagttcctgttctccctgtacagaaatgactacctgagaatcacaacaaacgaccctaccgaaaacgctcctatgatctacagattcgtgggcaccaagaaggatctgaataacgtgatcgaagtgaagcccatttactgtgacaagatcgacaaacctcagatcgaaaagaccatcggcaaaaaggtgaccaagatcgagaagttcgacgtggatgttctggggaacaagatcgccaagaagcgggacaagtgcctgcagctggaattg

SEQ ID NO: 18 - DNA, 3363 bp, synthetic construct
  source 1..3363: mol_type = other DNA
atgatccccaatcccctgctgggcctggacaagcggactgccgacggcagtgagttcgagtctcccaagaaaaagaggaaagtgggatccgccaagtacatcctgggactggatatgggcatcaccagcgtgggctggggcctgatcgactacgacagcggaaagatcgtggataagggcgtccggctgtttaaagaggcaaccgccgaggataatctgaacagaaggacacacagaggcagccggcggctgaagcgcagacggcagcaaagaatcatcgagctgaaaagactgctgaagaaggaaaacatcctgacagatgactacacccctctgaataatgtgtactacctgagatgcaaaggcctgaacgagaagctgaccaacgctgaactggtgtctgtgctgctgcacatcgccaagatacggggcagcagccttgaggtcgtggaagatgacgagaagaaggcgaatgaggacaagagctccaaggccaatctgagcgctaacgaggtgaagctcagaacatctaaggaggagatctgcgagaagcagtacaaggacctaatagagaacggaaaggtccggggcatcaataacatctataagtctagctcctacgaatcagagctgaagaaaatcctggccaatcagggcctgagcaaagaggtgaacgacaagatcatcaacatcattttccgccggcgagacttcaacgaaggccccgggagcatggaaagccctacaatctacggcagattcatccctcacgagaacggcgaggttgaagtgatccacagcatgatcgaaaagatgcagggcaaatgcagcatctaccagaacgagcctagagccgctaagatgagcttcacagccgacctgttcaatttcctgaacgacctgaacaacctggaaatccagagagccgataactacgacaactacattacccctgatgagaaaaagctgatcatcaacgagtacatcatcaagaaaggcaagatcaccattcagaaactctgcgacgtgctgcaagtgaagaaggaaaatatcaccggcttccgggtgaacaagggcgaacctatcctgacagagtttaagggctaccaaaaggtgctgaaggccctgaaccggatcatcgacaccgagatcgtggacgccgtgatcgacgtgctgaccatgcacaaagacgtggaaaagagaaaggaggagatcaaaaagatcaacaacaccgagttttctgaagaggatatcgtgaagctgtctgaaatcactggaatccagcagtaccattctctgagctacaaagccatgaaagtgatcatccctgagctgtacgagaacccagagaaccagatggaagtgatcaccagactgaacctggttaagacaagcatcgacgacctgaagggaagaaccaccattccatcttacagcaatagaatctatagccccgtggccaagagggtccataatgaaactattaaggtgatcaacgccgtgatcaagaaatacggcgacctggattccattgtgatcgaaatggccagagatagaaacaccgccgaagagcggaagaacatcgaacaggcccagaagcggaatcaagagaatagggaaaaggctaaagaaatcctgaacgtgaactacagctttagcaagctggacagcaagctggtccagaaaattatgctgtacaaccagcaggacggaaagagcatgtacagcggcaagcccatcaacctgaacgtgctgatctccgatcccaatgcttacgagatcgaccacatcatccccttgtccattagtctggatgatagcatcgccaacaaagtgctggtgtacagaagcgagaaccagcagaagctgaacaagacccctctgcagtacctgagatctggcaatagcaacggctggagcgtggacgagttcagagaggtggtgatcaagatgtacaacgacaaaaaaatctctctgaagaagctgcagaacctgctctgtgaaaaggacattaccaagcaggacgtgcggaaagagttcatcgaacggaacctggtggacacccggtacgccagcagagtggtgctgggcctgctgaaggactacttcaaggccaacaacaagaacaccaaggtctttaccatcagaggccagatcaccagccagttcagaaagagcagaaacctggtgaaatctagggattgtttctaccaccacatcgtggacgccctgatcatcgcctacttcagaaagtttaactatatcaatgaactgatgcagtacaacttcaactacaagggaaagattgacgaggagacaggcgagctgatccagtacaatgacaccctgttcagcaccaaggacgatgactttaccatccaactgagaaagctgaacgacctgaacaagtacagcgaggatatcaagatcagccacaaggtggatagaaagcctaaccggaaattctccgacgagacaatctacagcgccatcaaaatcggaaacgactacatgaagatatccaagtataaggatatctatggagaggaaggcaaaaagttcgccaagaaaatcaaggacaacctgcaaaaagaaaatgagctggatatcttcctgatgaagaaagtggaccccgagacattcaacttactagtgagcatcgtgaagaacacaatctgcggcgagaaggagaaccccttcgtaaaatattgcgaggaaaataatatcgacttcatcagaaaaaagtctaagaacggcaatggccctatcatccggtccatcagatactacgatggcaaagttaacagctaccaggacatcagccagaagtacagcaacctgagcgatgacaagaaggttgtgcggctgcacatctctccttaccggaccgacttctacctgaccaaggaaggcaactacaagttcctgaccatcaagtacgccaacatcaaggagaaccacaacggctactatatccctaaggaagtgtacgagacactgaagaagtctaagaagatcaacgacagcgacgagttcctgttctccctgtacagaaatgactacctgagaatcacaacaaacgaccctaccgaaaacgctcctatgatctacagattcgtgggcaccaagaaggatctgaataacgtgatcgaagtgaagcccatttactgtgacaagatcgacaaacctcagatcgaaaagaccatcggcaaaaaggtgaccaagatcgagaagttcgacgtggatgttctggggaacaagatcgccaagaagcgggacaagtgcctgcagctggaattgtctagaaagcggacagcagacggctccgaatttgaaagccctaagaaaaagagaaaggtgtga

SEQ ID NO: 19 - protein, 1095 aa, synthetic construct
IFGLDVGVASLGWAVIGEDYKVIETGVNSFKSADASMNVERRGFRQLRRLHRRKKNRINDFNNLWVKTGMRIPSQKCNNQIELRVKGLKDKLTEEEIYFVLVNMLKHRGISYLEDAVDESNKNESKYEKGINENQKLLQEGKFPCEIQLSRLEKHGQYRGEIVENDIILGNVFTTKAYRKEVVALLENQTKSHQFITDEFKSAYLNIFNRKRKYYEGPGNELSRTDYGIYTTVIDESTGEYKTEDNIFDKLIGKCSVYKNLRRAAGASYTSQEFNLLNDLNNLTVNGRKLEEVEKHRIVEIIKTSNTVNMKNIIKKVINENINTLSGARIDKDGKEIFHKFEQYNKIRKELEKIGGDITNFSREDLDEIGEILTLNTEKEAILSALRKIPCNDQEKECFFNLRRSNPGMFNKWQSFSLELMNELIPEMYLQSKNQMQLLTDMGVFKLSSDRYNGYKYIPKEVVTQEIYNPVVKRSIRITIDVMNALIKKYGYPEKIVIEMPRDKNSDDEKNRIKEEQKKNEREITEVLEKIKNEYGIVITDEHRKRQNQLNLKLKLWNEQDGRCLYSGKEIDINDLLYDPSKFEIDHIIPRSISFDDSRSNKVLVYYTENKKKGNLTPYMYLIGQNRDWDWHKYMNKVLELKANAKISKSKVDKLLFTDEITKIDVLKGFVSRNINDTRYASKVILNALQSFMQSKNVDTKVATIKGSFTHQMRKVLRLEKNRNESFAHHAIDAMLICYSQMGFDTYRKLQETFIDFDNEDITDKNLWDRYMDDRVCEEYLYNKKWRMIKENIQNAEKNIKYWYKVDSKVNRGLCNQTIVGTREINGEIYKINKLNLYTPDGYNRFKKIIDSGKEKDFLMYSNDRRTFNDILKIYRQYNDSTNAFMQYYNETGDFVRKYAKNHNGCRIENLKYYEKKIESCIDISHKYGHDVGSKKVVLLGLNPYRMDVYYNTNSNKYYFVGVKFSDLRFQGGTYLVDKDMYSQALINEKMLQPGEDISMLQSKGYEFRLTFYKNEIIKYEKKGEMFVERFLSRTSQDSNGIETKPINAEKFKNKDRNIVKLAKTKFVSKIRTDILGNQYECLKEEFKLMIDNVD

SEQ ID NO: 20 - protein, 1096 aa, synthetic construct
MIFGLDVGVASLGWAVIGEDYKVIETGVNSFKSADASMNVERRGFRQLRRLHRRKKNRINDFNNLWVKTGMRIPSQKCNNQIELRVKGLKDKLTEEEIYFVLVNMLKHRGISYLEDAVDESNKNESKYEKGINENQKLLQEGKFPCEIQLSRLEKHGQYRGEIVENDIILGNVFTTKAYRKEVVALLENQTKSHQFITDEFKSAYLNIFNRKRKYYEGPGNELSRTDYGIYTTVIDESTGEYKTEDNIFDKLIGKCSVYKNLRRAAGASYTSQEFNLLNDLNNLTVNGRKLEEVEKHRIVEIIKTSNTVNMKNIIKKVINENINTLSGARIDKDGKEIFHKFEQYNKIRKELEKIGGDITNFSREDLDEIGEILTLNTEKEAILSALRKIPCNDQEKECFFNLRRSNPGMFNKWQSFSLELMNELIPEMYLQSKNQMQLLTDMGVFKLSSDRYNGYKYIPKEVVTQEIYNPVVKRSIRITIDVMNALIKKYGYPEKIVIEMPRDKNSDDEKNRIKEEQKKNEREITEVLEKIKNEYGIVITDEHRKRQNQLNLKLKLWNEQDGRCLYSGKEIDINDLLYDPSKFEIDHIIPRSISFDDSRSNKVLVYYTENKKKGNLTPYMYLIGQNRDWDWHKYMNKVLELKANAKISKSKVDKLLFTDEITKIDVLKGFVSRNINDTRYASKVILNALQSFMQSKNVDTKVATIKGSFTHQMRKVLRLEKNRNESFAHHAIDAMLICYSQMGFDTYRKLQETFIDFDNEDITDKNLWDRYMDDRVCEEYLYNKKWRMIKENIQNAEKNIKYWYKVDSKVNRGLCNQTIVGTREINGEIYKINKLNLYTPDGYNRFKKIIDSGKEKDFLMYSNDRRTFNDILKIYRQYNDSTNAFMQYYNETGDFVRKYAKNHNGCRIENLKYYEKKIESCIDISHKYGHDVGSKKVVLLGLNPYRMDVYYNTNSNKYYFVGVKFSDLRFQGGTYLVDKDMYSQALINEKMLQPGEDISMLQSKGYEFRLTFYKNEIIKYEKKGEMFVERFLSRTSQDSNGIETKPINAEKFKNKDRNIVKLAKTKFVSKIRTDILGNQYECLKEEFKLMIDNVD

SEQ ID NO: 21 - protein, 1145 aa, synthetic construct
MIPNPLLGLDKRTADGSEFESPKKKRKVGSIFGLDVGVASLGWAVIGEDYKVIETGVNSFKSADASMNVERRGFRQLRRLHRRKKNRINDFNNLWVKTGMRIPSQKCNNQIELRVKGLKDKLTEEEIYFVLVNMLKHRGISYLEDAVDESNKNESKYEKGINENQKLLQEGKFPCEIQLSRLEKHGQYRGEIVENDIILGNVFTTKAYRKEVVALLENQTKSHQFITDEFKSAYLNIFNRKRKYYEGPGNELSRTDYGIYTTVIDESTGEYKTEDNIFDKLIGKCSVYKNLRRAAGASYTSQEFNLLNDLNNLTVNGRKLEEVEKHRIVEIIKTSNTVNMKNIIKKVINENINTLSGARIDKDGKEIFHKFEQYNKIRKELEKIGGDITNFSREDLDEIGEILTLNTEKEAILSALRKIPCNDQEKECFFNLRRSNPGMFNKWQSFSLELMNELIPEMYLQSKNQMQLLTDMGVFKLSSDRYNGYKYIPKEVVTQEIYNPVVKRSIRITIDVMNALIKKYGYPEKIVIEMPRDKNSDDEKNRIKEEQKKNEREITEVLEKIKNEYGIVITDEHRKRQNQLNLKLKLWNEQDGRCLYSGKEIDINDLLYDPSKFEIDHIIPRSISFDDSRSNKVLVYYTENKKKGNLTPYMYLIGQNRDWDWHKYMNKVLELKANAKISKSKVDKLLFTDEITKIDVLKGFVSRNINDTRYASKVILNALQSFMQSKNVDTKVATIKGSFTHQMRKVLRLEKNRNESFAHHAIDAMLICYSQMGFDTYRKLQETFIDFDNEDITDKNLWDRYMDDRVCEEYLYNKKWRMIKENIQNAEKNIKYWYKVDSKVNRGLCNQTIVGTREINGEIYKINKLNLYTPDGYNRFKKIIDSGKEKDFLMYSNDRRTFNDILKIYRQYNDSTNAFMQYYNETGDFVRKYAKNHNGCRIENLKYYEKKIESCIDISHKYGHDVGSKKVVLLGLNPYRMDVYYNTNSNKYYFVGVKFSDLRFQGGTYLVDKDMYSQALINEKMLQPGEDISMLQSKGYEFRLTFYKNEIIKYEKKGEMFVERFLSRTSQDSNGIETKPINAEKFKNKDRNIVKLAKTKFVSKIRTDILGNQYECLKEEFKLMIDNVDSRKRTADGSEFESPKKKRKV

SEQ ID NO: 22 - DNA, 3291 bp, synthetic construct
  source 1..3291: mol_type = other DNA
atgatatttggattagatgttggagttgcgtctttaggatgggcagttataggtgaagattataaagtaatagaaacaggtgtaaattcttttaagtcggcagatgcaagcatgaatgttgaaagacgaggatttaggcaattaagaagattgcatagaagaaagaagaatagaataaatgattttaataatttgtgggttaagacggggatgagaatacctagtcaaaaatgtaacaaccaaatcgaactcagagtaaaaggcttaaaggataaattaaccgaagaagagatttattttgtccttgtaaatatgttaaagcataggggaatatcatatcttgaagatgcagtagatgaatccaacaagaacgaaagcaaatatgaaaaaggaataaatgaaaatcaaaagttattgcaagagggcaaattcccttgtgaaatacagttaagtaggcttgaaaagcatggtcaatatagaggggaaattgtagaaaatgatattatattggggaacgtttttacaacaaaagcttatagaaaagaggtggtagcattattggaaaatcagacaaagtcacatcagtttatcacagatgagtttaagtcagcttatttaaatatatttaatagaaaaagaaaatattatgaaggaccgggaaatgaattatcaaggacggattatggaatatatacaactgtgatagacgaaagtacaggtgaatataaaacggaggataacatatttgataagttaattggaaaatgttcggtttataagaatttaagaagagctgcgggagcatcatatacatcacaggaatttaatcttttaaatgacttaaataacctgaccgttaatggaagaaagctagaggaagtggagaaacacagaatagttgaaattattaaaacatcaaacactgtaaatatgaaaaatattataaaaaaagtgataaatgagaatattaatacattgtcaggtgctagaatcgataaagatggaaaagaaatttttcacaaatttgagcaatataacaaaatacgaaaagaattagaaaaaataggaggggatataacaaatttttctagagaggatttggatgaaatcggagaaattttgacactaaatacagaaaaagaggcaatcttatcggcgttgagaaaaataccatgcaacgatcaggagaaagagtgtttctttaatttaagacgctcaaatcctggtatgtttaataagtggcagtctttttcgctggaattaatgaacgaacttattcctgaaatgtatttgcaatcaaagaatcagatgcaactacttactgatatgggagtgtttaaattatcaagtgacagatataatgggtataaatatatacccaaagaagtagtgacacaagaaatatataatccggtggttaaacgttcaataaggataacaatagatgttatgaatgctcttataaaaaaatatggttatccggaaaagatagttatagaaatgcctagagataaaaattctgatgatgaaaaaaataggataaaagaagaacaaaagaaaaatgaaagggagataacagaggtacttgaaaaaataaagaatgaatatggtattgtaataacagatgaacatcgcaaacgacagaaccagttaaatttgaagcttaaattatggaatgagcaggatgggaggtgtctttattcaggaaaagaaatagatattaatgatttactgtatgatccatctaaatttgaaatcgatcatattattccacgatcaatatcatttgatgatagtcgtagcaataaagttctggtgtactatacagaaaataaaaaaaagggaaatctaacgccatatatgtatcttattggtcaaaatagagattgggactggcataaatatatgaataaagttttggaattaaaagcaaatgccaaaatttccaaatctaaagttgataaattgttatttacagatgaaattactaagatagatgtattgaagggatttgtatcacgtaatattaatgatacgaggtatgcatctaaggttattcttaacgcgttgcagtcatttatgcaatcaaaaaatgtagacacaaaggtagcaactattaaaggaagctttactcatcaaatgaggaaagttctgagactggaaaagaatagaaatgaatccttcgcacatcatgctatagatgcaatgttaatttgttattcacaaatgggatttgatacatatcgaaagttacaagaaacatttattgattttgataatgaggatataacagataaaaatctgtgggatagatatatggatgatagggtctgcgaagaatatttgtataataaaaaatggagaatgattaaggaaaatatacaaaacgctgaaaaaaatattaagtattggtacaaagtagattctaaagtcaatagaggattatgcaatcaaacaatagtgggaacacgggaaataaatggagaaatatataaaattaataaacttaatctatatacgccagatggatataatagattcaaaaaaataatagattcaggtaaagaaaaagactttttgatgtatagtaatgacagaagaacatttaacgatatattaaaaatataccgtcaatataatgattcaacaaatgcttttatgcagtactacaatgaaacaggggattttgtcagaaaatatgctaaaaatcataatgggtgcagaatcgaaaatcttaaatattatgaaaaaaagattgagtcatgtatagatatttcgcataagtatgggcatgatgttggaagtaaaaaagttgttcttttagggcttaacccatatagaatggatgtatattataacactaatagcaataagtattattttgtaggagttaaattttcagatttaagatttcaaggtggtacatatttggttgataaagatatgtattcgcaagcgcttataaatgagaaaatgttacagcctggagaagacataagtatgttgcagagcaaaggctacgaattcaggttgacgttttacaaaaatgagataattaaatatgaaaaaaaaggagaaatgttcgttgaaaggtttctttcgaggacttcgcaagatagtaatggtatagaaacaaaacctataaacgccgaaaaatttaaaaataaagataggaatatagttaaacttgcaaaaacaaaatttgtcagtaaaatccgtacagatatattaggaaatcagtatgaatgtttaaaagaagaatttaaattaatgattgacaatgtggactag

SEQ ID NO: 23 - DNA, 3285 bp, synthetic construct
  source 1..3285: mol_type = other DNA
atcttcggcctggatgtaggagtggccagcctgggatgggccgtgatcggcgaggactacaaggtgatcgagactggcgttaattcttttaagtctgccgacgcctctatgaatgtggaaagaagaggatttagacagctgagaagattgcacagaagaaagaagaacagaatcaacgactttaacaatctgtgggtcaagacaggcatgagaatccctagccagaagtgcaacaaccagatcgagctgagagtgaagggcctgaaggacaagctcaccgaggaagagatctacttcgtgctggttaacatgctgaaacacaggggcatctcttacctggaggacgccgtggatgagtccaacaaaaatgagagcaagtacgagaagggcatcaacgaaaatcagaaactgctgcaggagggcaagttcccttgtgaaatccagctgagcagactggagaaacacggccagtataggggcgagatcgtggagaacgacatcatcctgggcaacgtgttcacaaccaaagcctacagaaaggaggtggtggccctgctggaaaaccagaccaagtctcaccagttcatcaccgatgagttcaagagcgcctatctgaacatctttaatagaaagcggaagtactacgagggacctggcaatgaactgagcagaaccgattacggcatctacaccacagtgattgacgagtccaccggcgaatacaagaccgaggacaacatcttcgacaagctgatcggcaagtgcagcgtgtacaagaacctgagaagagccgccggcgccagctacacctctcaagagttcaaccttctcaacgacctgaataacctgacagtgaacggcagaaagctcgaagaggtggaaaaacatagaatcgtcgagatcatcaagacttctaacaccgtgaacatgaagaatataatcaagaaggtgatcaacgaaaacatcaacaccctcagcggcgccagaatcgacaaggacggcaaggagatcttccacaagttcgagcaatacaacaagatccggaaagagctggagaagatcggaggcgatatcaccaacttcagcagagaggacctggacgagatcggcgagatcctgaccctgaacaccgagaaagaggccatcctgtctgccctgagaaagatcccatgcaacgaccaggaaaaagaatgcttcttcaacctgagaagatctaaccccggcatgttcaacaagtggcagtctttcagcctggaactgatgaacgaactgatccccgagatgtacctgcagagcaagaaccaaatgcagctgctgaccgatatgggggtctttaagctgagttctgatagatacaatggctacaagtacattcccaaggaagtggtgacccaggagatctataatcctgtggtgaaacggtccatcaggattacaatcgacgtgatgaacgccctgatcaagaaatatggataccctgagaagatcgtgatcgagatgccacgggacaaaaacagcgacgacgagaagaacagaatcaaagaagagcagaagaagaacgagcgcgagatcacagaagtgctcgaaaagatcaagaacgaatacggaattgtgatcaccgatgagcacagaaagagacagaaccagctgaatctcaaactgaagctgtggaacgaacaagacggaagatgcctgtactccggcaaagaaatcgacatcaacgatctcctgtacgacccctccaagtttgagatcgatcacatcattcctagatccatcagcttcgacgatagccggagcaacaaggtgctggtgtattacaccgagaacaagaagaagggcaacctgaccccctacatgtacctgatcggacagaaccgggattgggactggcacaagtatatgaacaaagtgctggaactgaaggccaatgctaagatcagcaagagcaaggtggacaagctgctgttcacagacgagataaccaagatcgacgtgctgaagggtttcgtgagccggaacatcaacgacaccagatacgccagcaaggtgatcctgaacgcactgcagtctttcatgcaaagcaagaacgtggatacaaaggtggctacaatcaagggctcttttacccaccagatgcggaaggtgctgagactggaaaaaaacagaaatgagtccttcgcccaccacgccatagatgctatgttaatttgctacagccagatgggcttcgacacataccgcaagctgcaggagacatttatcgacttcgataacgaggatatcacagacaagaacctgtgggatcggtacatggatgacagagtgtgcgaggagtacctttacaacaagaaatggcggatgatcaaggaaaacattcagaatgccgaaaagaacatcaaatactggtacaaggtggactccaaggtgaaccgcggactttgtaaccagaccatcgtgggcacccgggagatcaacggcgagatctacaagataaataaactgaacctgtacacacctgatggctacaatagattcaagaaaattatcgacagcggcaaggaaaaagactttctgatgtacagcaatgacagacggaccttcaacgacatcctgaagatctaccggcagtacaacgacagcaccaacgccttcatgcagtactacaacgaaaccggcgacttcgtgcggaaatacgccaaaaaccacaacggctgtcggatcgagaatctgaagtattacgaaaagaagatcgagagctgcatcgatatcagccacaagtacggccatgatgttggcagcaagaaagtggtgctgctgggcctgaacccttacagaatggacgtgtactataataccaatagcaacaagtactatttcgtgggagtgaagttcagcgatctgcggtttcagggcggcacctacctggtggacaaggacatgtacagccaggccctgatcaacgaaaagatgctgcagcctggcgaggatattagcatgctgcaaagcaagggatacgagttcagactgacattctacaagaatgaaatcatcaagtacgagaagaagggcgaaatgttcgtggagagattcctgagcagaacctcacaggattccaacggcatcgagacaaagcctatcaacgctgagaaattcaaaaacaaagacagaaacatcgtgaagctggccaagaccaagttcgtgtcaaagatcagaaccgacatcctgggcaatcagtacgagtgtttaaaagaggagttcaagctgatgatcgacaacgtggac

SEQ ID NO: 24 - DNA, 3438 bp, synthetic construct
  source 1..3438: mol_type = other DNA
atgatccccaatcccctgctgggcctggacaagcggactgccgacggcagtgagttcgagtctcccaagaaaaagaggaaagtgggatccatcttcggcctggatgtaggagtggccagcctgggatgggccgtgatcggcgaggactacaaggtgatcgagactggcgttaattcttttaagtctgccgacgcctctatgaatgtggaaagaagaggatttagacagctgagaagattgcacagaagaaagaagaacagaatcaacgactttaacaatctgtgggtcaagacaggcatgagaatccctagccagaagtgcaacaaccagatcgagctgagagtgaagggcctgaaggacaagctcaccgaggaagagatctacttcgtgctggttaacatgctgaaacacaggggcatctcttacctggaggacgccgtggatgagtccaacaaaaatgagagcaagtacgagaagggcatcaacgaaaatcagaaactgctgcaggagggcaagttcccttgtgaaatccagctgagcagactggagaaacacggccagtataggggcgagatcgtggagaacgacatcatcctgggcaacgtgttcacaaccaaagcctacagaaaggaggtggtggccctgctggaaaaccagaccaagtctcaccagttcatcaccgatgagttcaagagcgcctatctgaacatctttaatagaaagcggaagtactacgagggacctggcaatgaactgagcagaaccgattacggcatctacaccacagtgattgacgagtccaccggcgaatacaagaccgaggacaacatcttcgacaagctgatcggcaagtgcagcgtgtacaagaacctgagaagagccgccggcgccagctacacctctcaagagttcaaccttctcaacgacctgaataacctgacagtgaacggcagaaagctcgaagaggtggaaaaacatagaatcgtcgagatcatcaagacttctaacaccgtgaacatgaagaatataatcaagaaggtgatcaacgaaaacatcaacaccctcagcggcgccagaatcgacaaggacggcaaggagatcttccacaagttcgagcaatacaacaagatccggaaagagctggagaagatcggaggcgatatcaccaacttcagcagagaggacctggacgagatcggcgagatcctgaccctgaacaccgagaaagaggccatcctgtctgccctgagaaagatcccatgcaacgaccaggaaaaagaatgcttcttcaacctgagaagatctaaccccggcatgttcaacaagtggcagtctttcagcctggaactgatgaacgaactgatccccgagatgtacctgcagagcaagaaccaaatgcagctgctgaccgatatgggggtctttaagctgagttctgatagatacaatggctacaagtacattcccaaggaagtggtgacccaggagatctataatcctgtggtgaaacggtccatcaggattacaatcgacgtgatgaacgccctgatcaagaaatatggataccctgagaagatcgtgatcgagatgccacgggacaaaaacagcgacgacgagaagaacagaatcaaagaagagcagaagaagaacgagcgcgagatcacagaagtgctcgaaaagatcaagaacgaatacggaattgtgatcaccgatgagcacagaaagagacagaaccagctgaatctcaaactgaagctgtggaacgaacaagacggaagatgcctgtactccggcaaagaaatcgacatcaacgatctcctgtacgacccctccaagtttgagatcgatcacatcattcctagatccatcagcttcgacgatagccggagcaacaaggtgctggtgtattacaccgagaacaagaagaagggcaacctgaccccctacatgtacctgatcggacagaaccgggattgggactggcacaagtatatgaacaaagtgctggaactgaaggccaatgctaagatcagcaagagcaaggtggacaagctgctgttcacagacgagataaccaagatcgacgtgctgaagggtttcgtgagccggaacatcaacgacaccagatacgccagcaaggtgatcctgaacgcactgcagtctttcatgcaaagcaagaacgtggatacaaaggtggctacaatcaagggctcttttacccaccagatgcggaaggtgctgagactggaaaaaaacagaaatgagtccttcgcccaccacgccatagatgctatgttaatttgctacagccagatgggcttcgacacataccgcaagctgcaggagacatttatcgacttcgataacgaggatatcacagacaagaacctgtgggatcggtacatggatgacagagtgtgcgaggagtacctttacaacaagaaatggcggatgatcaaggaaaacattcagaatgccgaaaagaacatcaaatactggtacaaggtggactccaaggtgaaccgcggactttgtaaccagaccatcgtgggcacccgggagatcaacggcgagatctacaagataaataaactgaacctgtacacacctgatggctacaatagattcaagaaaattatcgacagcggcaaggaaaaagactttctgatgtacagcaatgacagacggaccttcaacgacatcctgaagatctaccggcagtacaacgacagcaccaacgccttcatgcagtactacaacgaaaccggcgacttcgtgcggaaatacgccaaaaaccacaacggctgtcggatcgagaatctgaagtattacgaaaagaagatcgagagctgcatcgatatcagccacaagtacggccatgatgttggcagcaagaaagtggtgctgctgggcctgaacccttacagaatggacgtgtactataataccaatagcaacaagtactatttcgtgggagtgaagttcagcgatctgcggtttcagggcggcacctacctggtggacaaggacatgtacagccaggccctgatcaacgaaaagatgctgcagcctggcgaggatattagcatgctgcaaagcaagggatacgagttcagactgacattctacaagaatgaaatcatcaagtacgagaagaagggcgaaatgttcgtggagagattcctgagcagaacctcacaggattccaacggcatcgagacaaagcctatcaacgctgagaaattcaaaaacaaagacagaaacatcgtgaagctggccaagaccaagttcgtgtcaaagatcagaaccgacatcctgggcaatcagtacgagtgtttaaaagaggagttcaagctgatgatcgacaacgtggactctagaaagcggacagcagacggctccgaatttgaaagccctaagaaaaagagaaaggtgtga

SEQ ID NO: 25 - protein, 1070 aa, synthetic construct
AKYILGLDMGITSVGWGLIDYDSGKIVDKGVRLFKEATAEDNLKRRTHRGSRRLKRRRQQRIIELKRLLKKENILTDDYTPLNNVYYLRCKGLNEKLTNAELVSVLLHIAKIRGSSLEVVEDDEKKANEDKSSKANLSVNETKLRTSKEEICEKQYKDLIENGKVRGINNIYKSSSYESELKKILANQGLSKEVNDKIINIIFRRRDFNEGPGSMESPTIYGRFIPHENGEIEIIPSMIEKMQGKCSIYQNEPRAAKMSFTADLFNFLNDLNNLEIQRADNYDNYITPDEKKLIINEYIIKKGKITIQKLCDVLQVKKENITGFRVNKGEPILTEFKGYQKVLKALNRIIDTEIVDAVIDVLTKYKDVEKRKEEIKKINNAEFSEEDIVKLSEITGIQQYHSLSYKAMKVIIPELYENPENQMEVITRLNLVKTSIDDLKGRTTIPSYSDRIYSPVAKRVHNETIKVVNAVIKKYGDLDSIVIEMARDRNTAEERKNIEQAQKRNQENREKAKEILDVNYSFSKLDSKLVQKIMLYNQQDGKSMYSGKPINLNVLISDPNAYEIDHIIPLSISLDDSIANKVLVYRSENQQKLNNTPLQYLRSGNSNGWSVDEFREVVIKMYNDKKISLKKLQNLLCEKDITKQDVRKEFIERNLVDTRYASRVVLGLLKDYFKANNKNTKVFTIRGQITSQFRKSRNLVKSRDCFYHHIVDALIIAYFRKFNYINELMQYNFSYKVKIDEETGELIQYNDTLFSTKDDDFTIQLRKLNDLNKYSEDIKISHKVDRKPNRQFSDETIYSARKINNDYMKISKYKDIYGEEGKKFAKVIKDNLQKEKDLDKFLMKKVDPETFNLLVSIVINTTCGEKENPFVKYCEENNIDFIRKKSKNGNGPIIRSIRYYDGKVNSHLDISQKYVNLSDDKKVVLLQISPYRTDFYLTKEGNYKFLTIKYANIKENHNGYYIPKDVYESLKKSKKINDSDEFLFSLYRNDYLRITTNDPTENAPMIYRFVGTNNDLENKVEVKPIYCDKNEENRIMKTIGKKVTKIEKFDVDVLGNKIAKKRDKCLQLEL

SEQ ID NO: 26 - protein, 1071 aa, synthetic construct
VAKYILGLDMGITSVGWGLIDYDSGKIVDKGVRLFKEATAEDNLKRRTHRGSRRLKRRRQQRIIELKRLLKKENILTDDYTPLNNVYYLRCKGLNEKLTNAELVSVLLHIAKIRGSSLEVVEDDEKKANEDKSSKANLSVNETKLRTSKEEICEKQYKDLIENGKVRGINNIYKSSSYESELKKILANQGLSKEVNDKIINIIFRRRDFNEGPGSMESPTIYGRFIPHENGEIEIIPSMIEKMQGKCSIYQNEPRAAKMSFTADLFNFLNDLNNLEIQRADNYDNYITPDEKKLIINEYIIKKGKITIQKLCDVLQVKKENITGFRVNKGEPILTEFKGYQKVLKALNRIIDTEIVDAVIDVLTKYKDVEKRKEEIKKINNAEFSEEDIVKLSEITGIQQYHSLSYKAMKVIIPELYENPENQMEVITRLNLVKTSIDDLKGRTTIPSYSDRIYSPVAKRVHNETIKVVNAVIKKYGDLDSIVIEMARDRNTAEERKNIEQAQKRNQENREKAKEILDVNYSFSKLDSKLVQKIMLYNQQDGKSMYSGKPINLNVLISDPNAYEIDHIIPLSISLDDSIANKVLVYRSENQQKLNNTPLQYLRSGNSNGWSVDEFREVVIKMYNDKKISLKKLQNLLCEKDITKQDVRKEFIERNLVDTRYASRVVLGLLKDYFKANNKNTKVFTIRGQITSQFRKSRNLVKSRDCFYHHIVDALIIAYFRKFNYINELMQYNFSYKVKIDEETGELIQYNDTLFSTKDDDFTIQLRKLNDLNKYSEDIKISHKVDRKPNRQFSDETIYSARKINNDYMKISKYKDIYGEEGKKFAKVIKDNLQKEKDLDKFLMKKVDPETFNLLVSIVINTTCGEKENPFVKYCEENNIDFIRKKSKNGNGPIIRSIRYYDGKVNSHLDISQKYVNLSDDKKVVLLQISPYRTDFYLTKEGNYKFLTIKYANIKENHNGYYIPKDVYESLKKSKKINDSDEFLFSLYRNDYLRITTNDPTENAPMIYRFVGTNNDLENKVEVKPIYCDKNEENRIMKTIGKKVTKIEKFDVDVLGNKIAKKRDKCLQLEL

SEQ ID NO: 27 - protein, 1120 aa, synthetic construct
MIPNPLLGLDKRTADGSEFESPKKKRKVGSAKYILGLDMGITSVGWGLIDYDSGKIVDKGVRLFKEATAEDNLKRRTHRGSRRLKRRRQQRIIELKRLLKKENILTDDYTPLNNVYYLRCKGLNEKLTNAELVSVLLHIAKIRGSSLEVVEDDEKKANEDKSSKANLSVNETKLRTSKEEICEKQYKDLIENGKVRGINNIYKSSSYESELKKILANQGLSKEVNDKIINIIFRRRDFNEGPGSMESPTIYGRFIPHENGEIEIIPSMIEKMQGKCSIYQNEPRAAKMSFTADLFNFLNDLNNLEIQRADNYDNYITPDEKKLIINEYIIKKGKITIQKLCDVLQVKKENITGFRVNKGEPILTEFKGYQKVLKALNRIIDTEIVDAVIDVLTKYKDVEKRKEEIKKINNAEFSEEDIVKLSEITGIQQYHSLSYKAMKVIIPELYENPENQMEVITRLNLVKTSIDDLKGRTTIPSYSDRIYSPVAKRVHNETIKVVNAVIKKYGDLDSIVIEMARDRNTAEERKNIEQAQKRNQENREKAKEILDVNYSFSKLDSKLVQKIMLYNQQDGKSMYSGKPINLNVLISDPNAYEIDHIIPLSISLDDSIANKVLVYRSENQQKLNNTPLQYLRSGNSNGWSVDEFREVVIKMYNDKKISLKKLQNLLCEKDITKQDVRKEFIERNLVDTRYASRVVLGLLKDYFKANNKNTKVFTIRGQITSQFRKSRNLVKSRDCFYHHIVDALIIAYFRKFNYINELMQYNFSYKVKIDEETGELIQYNDTLFSTKDDDFTIQLRKLNDLNKYSEDIKISHKVDRKPNRQFSDETIYSARKINNDYMKISKYKDIYGEEGKKFAKVIKDNLQKEKDLDKFLMKKVDPETFNLLVSIVINTTCGEKENPFVKYCEENNIDFIRKKSKNGNGPIIRSIRYYDGKVNSHLDISQKYVNLSDDKKVVLLQISPYRTDFYLTKEGNYKFLTIKYANIKENHNGYYIPKDVYESLKKSKKINDSDEFLFSLYRNDYLRITTNDPTENAPMIYRFVGTNNDLENKVEVKPIYCDKNEENRIMKTIGKKVTKIEKFDVDVLGNKIAKKRDKCLQLELSRKRTADGSEFESPKKKRKV

SEQ ID NO: 28 - DNA, 3216 bp, synthetic construct
  source 1..3216: mol_type = other DNA
gtggcaaaatatatattaggactagatatggggattacttcggtaggttggggcctaattgactatgattctggaaaaatagttgataaaggggttagactttttaaagaagcaactgcggaagacaatttaaaaagacgtacacatcgtggatcaagaaggttaaaaagacgtagacaacaaagaataattgaattaaaacgattattgaaaaaagaaaacatccttacagatgattatactcctttaaataatgtttactatcttagatgtaaaggtttaaatgaaaaattaacaaatgccgaacttgtatcagtattattacatatagcaaaaataagaggaagttcattagaagtagttgaagatgatgaaaaaaaggcaaatgaagataaaagtagtaaagccaatttatcagtaaatgaaacaaaactaaggacttcaaaagaagaaatttgtgaaaaacagtataaagatctaatcgaaaatggtaaagttcgtggaataaataatatttataaaagttcttcatatgagtcagaactcaaaaaaattttagctaatcaaggcttgtcaaaagaagttaatgataagataataaatataatttttagaagacgtgactttaatgaaggaccaggaagcatggaatcgcctactatatatggtagatttataccacatgaaaatggtgagatagaaattatacctagtatgattgaaaaaatgcaagggaaatgttctatataccaaaatgaacctcgtgcagcaaaaatgtcatttacagctgatttgtttaatttcttaaacgatttaaacaatttagaaatacagcgtgctgataactatgataattatattaccccagacgaaaaaaaactaataataaatgagtatatcataaaaaaaggtaaaattacaattcaaaaattatgcgatgtactacaggtaaaaaaagaaaatattactggctttagagttaacaaaggtgaaccaatactcactgaatttaaaggttatcaaaaagtattaaaagctttaaatagaataattgatactgaaattgttgatgcggttattgatgttttaacaaaatataaagatgtagaaaaaagaaaagaagaaattaaaaaaataaacaatgcagagtttagtgaggaagacattgtaaaattatctgaaataacaggtatacaacagtatcattcattgtcttataaagcaatgaaagtaataatacctgaattatatgaaaatcctgaaaatcaaatggaagtaattacaagattaaatctagtgaaaacttcaatagatgatttgaaaggaagaacaacaataccttcatatagcgacagaatatattcccctgtagctaaacgtgtacataatgagacaatcaaagtagtaaatgcggtaataaaaaaatatggagatttagatagtattgttatagaaatggcaagggatagaaatactgctgaagaaaggaaaaatattgaacaagcccaaaaacgaaatcaagaaaatagagaaaaagcaaaagagatactagatgttaattatagtttttcgaaacttgattcaaaattagttcaaaagatcatgctttataatcaacaagatggtaaaagtatgtatagtggaaaaccaataaacttaaatgttttaataagtgaccctaatgcatacgagatagatcatatcataccattatcaatttcgcttgatgattcaattgccaataaagttctggtgtatagaagtgaaaaccaacaaaaattaaataatacaccacttcaatacttacgtagtggtaattcaaatggttggagcgtcgacgaattccgtgaagttgtaataaaaatgtataatgataaaaaaatatcactaaaaaaattacaaaacttactatgtgaaaaagatataacaaaacaagatgtaagaaaagaatttattgaaagaaatttagttgacacaagatatgcaagtagggtagttttgggacttctaaaagattatttcaaagccaacaataaaaatactaaagtatttacaataagaggccaaataacatcacagttcagaaaaagtagaaacttggtgaaaagtagagattgcttttatcatcatatagtagatgcattaataattgcatattttagaaaattcaattacattaatgaattaatgcaatacaattttagttataaagtgaaaattgatgaagaaacaggagaattaatccaatataatgatacgttgttttcaacgaaggatgatgatttcacaatacaattaagaaaactaaatgaccttaataaatatagtgaagatatcaaaatttcgcataaagttgatagaaagcctaatagacagtttagtgatgaaacaatctattctgcgagaaaaatcaataacgattatatgaaaatttcgaaatataaagatatttatggtgaagaaggtaaaaaatttgcaaaagtaattaaagataatctccaaaaagaaaaagacttagataaattcttaatgaagaaggtagatccagaaacttttaatttattagttagcattgttataaatacaacatgtggtgaaaaagaaaacccttttgttaaatattgtgaagaaaacaatatagattttattagaaaaaaatctaaaaatggtaatggtccgattattaggtctattagatattatgatggtaaagtaaatagtcacttggatatctcacaaaaatatgtaaacttatctgatgacaaaaaagtggtgcttttacaaatttcaccatatagaactgatttttacttaactaaagaaggaaactataaatttctaacaattaaatatgctaatatcaaagaaaaccataatggatattatatacccaaagatgtttacgaatcattaaagaaatcaaagaaaataaatgatagtgatgaatttttattttctctatatcgaaatgattacctaagaattacaacaaatgatcctactgaaaatgctcctatgatttatcggtttgtaggtacaaataatgatttagaaaacaaagttgaagtaaaaccaatttattgtgataaaaatgaagaaaatcgtattatgaaaaccataggtaaaaaagtgactaaaattgaaaagtttgatgttgatgtactaggtaataaaattgctaaaaaaagagataaatgcttgcaattagagctgtag

SEQ ID NO: 29 - DNA, 3210 bp, synthetic construct
  source 1..3210: mol_type = other DNA
gctaagtacatcctgggactggacatgggcatcacaagcgtgggctggggcctgatcgattacgacagcggcaagatcgtggacaagggcgtgcggctgtttaaggaggccacagccgaggacaacctgaagagaaggacccacagaggctcccggagactgaagcggcggagacagcaaagaattatagagctgaagcgcctgctgaaaaaagaaaatatcctgacagatgactacacccctctgaacaatgtgtactatctgaggtgtaaaggactcaacgagaaactgacaaacgccgagctggtgtccgtcctgcttcatatcgccaagattcggggcagcagcctggaagtggtcgaagatgacgaaaaaaaggccaacgaagataagagcagcaaggccaacctgtccgtgaatgaaaccaagctgagaacaagcaaagaagagatctgcgagaagcaatacaaagacctgatcgagaacggcaaggtcagaggaatcaacaacatctataagagctctagctatgaaagcgagctgaagaaaattctggccaatcagggcctgagcaaggaggtgaacgataagatcatcaacatcattttcagacggcgggacttcaacgaaggccccggcagcatggagagcccaacaatctacggcaggttcatcccacacgagaacggcgaaatcgaaatcattccttctatgatcgaaaagatgcagggcaagtgcagcatctaccagaacgaacctagagccgcgaagatgagcttcaccgccgatctgttcaatttcctgaacgacctgaacaatttggagatccagagagccgacaactacgacaactacatcacccctgacgagaagaaactgatcatcaacgagtacatcattaagaagggcaagatcaccatccagaagctgtgcgacgtgctgcaagtgaagaaggagaacatcaccggattcagagtgaacaagggcgaacccatcctgaccgagttcaagggatatcagaaggtgctgaaagctctgaacagaatcattgataccgagatcgtggatgccgtgatcgatgtgctgaccaaatacaaggacgtggaaaaacggaaagaggaaatcaaaaagatcaacaatgctgagttcagcgaggaagatatcgtgaagctgagcgagatcacaggcatccagcagtaccacagcctgtcctacaaggccatgaaggtgatcatccctgagctgtatgagaaccccgagaaccagatggaagtgatcaccagactcaatctggtgaaaaccagcatcgacgacctgaagggcagaaccaccatccccagctactccgaccggatctacagccctgtggccaaaagagtgcacaacgagacaatcaaggtggtgaacgcagtgatcaagaagtacggcgaccttgacagcatcgtgatcgagatggccagagacagaaacaccgccgaagaaagaaagaacatcgaacaggcccagaagcggaaccaggagaatagagagaaagctaaagaaatcctggacgtaaactactctttttcaaagctggattctaagctggtccagaaaatcatgctgtacaatcaacaagacggaaagtccatgtacagcggcaagcccatcaatctgaacgtcctgatcagcgaccctaacgcctacgagatcgaccacatcatccccctgtctatcagcctggacgacagcatcgccaacaaagtgctggtttacagaagcgagaaccagcagaagctgaacaacacacccctgcagtacctgagaagcggcaatagcaacggctggagcgtggacgagttcagagaagtggtgatcaagatgtacaacgacaaaaaaatttccctcaaaaaactgcagaacctgctgtgcgagaaggacatcaccaagcaggacgtgcgaaaggaatttatcgagcggaacctggtcgatacaagatacgccagccgggtggtgctgggcctgctgaaggactatttcaaggccaataacaagaacaccaaggttttcacaatcagaggacagatcaccagccagtttcggaagagcagaaatctggtgaagagccgggattgcttctaccaccacatcgtggatgccctgatcatcgcttacttccggaagttcaactacattaacgagcttatgcagtacaacttcagttacaaagtgaagatcgatgaggagacaggcgaactgatccagtataatgataccctgttcagcaccaaggatgacgattttacaatccaactgagaaaacttaacgacctgaacaagtacagtgaggatatcaagatcagccacaaggtggatagaaagcctaatagacagttttctgacgagacaatctacagcgccagaaagatcaacaacgactacatgaagatcagcaagtacaaggacatctatggcgaggagggcaagaagttcgctaaggttatcaaggacaacctgcagaaggagaaggacctggacaagtttctgatgaagaaggtagatcctgaaaccttcaacctgctggtgtccatcgtgattaacaccacttgcggcgagaaggagaaccctttcgtgaagtactgtgaagagaacaacatcgacttcatccggaagaagagcaagaacgggaacggccctatcatcagaagcatcagatactacgacggcaaagtgaacagccacctggacatctctcagaagtacgtgaacctctctgacgacaagaaggtggtgctgctgcagatcagcccctaccggaccgacttctacctgaccaaagagggcaactacaaattcctgaccattaagtacgccaacatcaaggaaaatcacaacggctactacatccctaaggacgtgtacgagagcctgaagaagtctaagaagatcaacgacagcgatgagttcctgttttctctgtacagaaatgactacctcaggatcactacaaacgaccctacagagaacgctcctatgatctatagattcgtgggcaccaacaacgacctggaaaacaaggttgaggtcaaacctatctattgcgacaaaaatgaagagaaccggatcatgaaaaccatcggcaagaaggtgaccaagatcgagaagttcgacgtggatgtgctgggaaacaagatcgccaaaaagagagacaagtgtctgcagctggagctg

SEQ ID NO: 30 - DNA, 3363 bp, synthetic construct
  source 1..3363: mol_type = other DNA
atgatccccaatcccctgctgggcctggacaagcggactgccgacggcagtgagttcgagtctcccaagaaaaagaggaaagtgggatccgctaagtacatcctgggactggacatgggcatcacaagcgtgggctggggcctgatcgattacgacagcggcaagatcgtggacaagggcgtgcggctgtttaaggaggccacagccgaggacaacctgaagagaaggacccacagaggctcccggagactgaagcggcggagacagcaaagaattatagagctgaagcgcctgctgaaaaaagaaaatatcctgacagatgactacacccctctgaacaatgtgtactatctgaggtgtaaaggactcaacgagaaactgacaaacgccgagctggtgtccgtcctgcttcatatcgccaagattcggggcagcagcctggaagtggtcgaagatgacgaaaaaaaggccaacgaagataagagcagcaaggccaacctgtccgtgaatgaaaccaagctgagaacaagcaaagaagagatctgcgagaagcaatacaaagacctgatcgagaacggcaaggtcagaggaatcaacaacatctataagagctctagctatgaaagcgagctgaagaaaattctggccaatcagggcctgagcaaggaggtgaacgataagatcatcaacatcattttcagacggcgggacttcaacgaaggccccggcagcatggagagcccaacaatctacggcaggttcatcccacacgagaacggcgaaatcgaaatcattccttctatgatcgaaaagatgcagggcaagtgcagcatctaccagaacgaacctagagccgcgaagatgagcttcaccgccgatctgttcaatttcctgaacgacctgaacaatttggagatccagagagccgacaactacgacaactacatcacccctgacgagaagaaactgatcatcaacgagtacatcattaagaagggcaagatcaccatccagaagctgtgcgacgtgctgcaagtgaagaaggagaacatcaccggattcagagtgaacaagggcgaacccatcctgaccgagttcaagggatatcagaaggtgctgaaagctctgaacagaatcattgataccgagatcgtggatgccgtgatcgatgtgctgaccaaatacaaggacgtggaaaaacggaaagaggaaatcaaaaagatcaacaatgctgagttcagcgaggaagatatcgtgaagctgagcgagatcacaggcatccagcagtaccacagcctgtcctacaaggccatgaaggtgatcatccctgagctgtatgagaaccccgagaaccagatggaagtgatcaccagactcaatctggtgaaaaccagcatcgacgacctgaagggcagaaccaccatccccagctactccgaccggatctacagccctgtggccaaaagagtgcacaacgagacaatcaaggtggtgaacgcagtgatcaagaagtacggcgaccttgacagcatcgtgatcgagatggccagagacagaaacaccgccgaagaaagaaagaacatcgaacaggcccagaagcggaaccaggagaatagagagaaagctaaagaaatcctggacgtaaactactctttttcaaagctggattctaagctggtccagaaaatcatgctgtacaatcaacaagacggaaagtccatgtacagcggcaagcccatcaatctgaacgtcctgatcagcgaccctaacgcctacgagatcgaccacatcatccccctgtctatcagcctggacgacagcatcgccaacaaagtgctggtttacagaagcgagaaccagcagaagctgaacaacacacccctgcagtacctgagaagcggcaatagcaacggctggagcgtggacgagttcagagaagtggtgatcaagatgtacaacgacaaaaaaatttccctcaaaaaactgcagaacctgctgtgcgagaaggacatcaccaagcaggacgtgcgaaaggaatttatcgagcggaacctggtcgatacaagatacgccagccgggtggtgctgggcctgctgaaggactatttcaaggccaataacaagaacaccaaggttttcacaatcagaggacagatcaccagccagtttcggaagagcagaaatctggtgaagagccgggattgcttctaccaccacatcgtggatgccctgatcatcgcttacttccggaagttcaactacattaacgagcttatgcagtacaacttcagttacaaagtgaagatcgatgaggagacaggcgaactgatccagtataatgataccctgttcagcaccaaggatgacgattttacaatccaactgagaaaacttaacgacctgaacaagtacagtgaggatatcaagatcagccacaaggtggatagaaagcctaatagacagttttctgacgagacaatctacagcgccagaaagatcaacaacgactacatgaagatcagcaagtacaaggacatctatggcgaggagggcaagaagttcgctaaggttatcaaggacaacctgcagaaggagaaggacctggacaagtttctgatgaagaaggtagatcctgaaaccttcaacctgctggtgtccatcgtgattaacaccacttgcggcgagaaggagaaccctttcgtgaagtactgtgaagagaacaacatcgacttcatccggaagaagagcaagaacgggaacggccctatcatcagaagcatcagatactacgacggcaaagtgaacagccacctggacatctctcagaagtacgtgaacctctctgacgacaagaaggtggtgctgctgcagatcagcccctaccggaccgacttctacctgaccaaagagggcaactacaaattcctgaccattaagtacgccaacatcaaggaaaatcacaacggctactacatccctaaggacgtgtacgagagcctgaagaagtctaagaagatcaacgacagcgatgagttcctgttttctctgtacagaaatgactacctcaggatcactacaaacgaccctacagagaacgctcctatgatctatagattcgtgggcaccaacaacgacctggaaaacaaggttgaggtcaaacctatctattgcgacaaaaatgaagagaaccggatcatgaaaaccatcggcaagaaggtgaccaagatcgagaagttcgacgtggatgtgctgggaaacaagatcgccaaaaagagagacaagtgtctgcagctggagctgtctagaaagcggacagcagacggctccgaatttgaaagccctaagaaaaagagaaaggtgtga

SEQ ID NO: 31 - protein, 1070 aa, synthetic construct
AKYILGLDMGITSVGWGLIDYDSGKIVDKGVRLFKEATAEDNLNRRTHRGSRRLKRRRQQRIIELKRLLKKENILTDDYTPLNNVYYLRCKGLNEKLTNAELVSVLLHIAKIRGSSLEVVEDDEKKANEDKSSKANLSVNETKLRTSKEEICEKQYKDLIENGKVRGINNIYKSSSYESELKKILDNQGLSKEVNDKIINIIFRRRDFNEGPGSIESPTIYGRFIPHENGEIEIIPSMIEKMQGKCSIYQDEPRAPKMSFTADLFNFLNDLNNLEIQRADNYDNYITPDEKKLIINEYIIKKGKITIQKLCDVLQVKKENITGFRVNKGEPILTEFKGYQKVLKALNRIIDTEIVDAVIDVLTKYKDVEKRKEEIKKINNAEFSEEDIVKLSEITGIQQYHSLSYKAMKVIIPELYENPENQMEVITRLNLVKTSIDDLKGRTTIPSYSDRIYSPVAKRVHNETIKVVNAVIKKYGDLDSIVIEMARDRNTAEERKNIEQAQKRNQENREKAKEILDANHSFSKLDSKLVQKIMLYNQQDGKSMYSGKPINLNVLISDPNAYEIDHIIPLSISLDDSIANKVLVYRSENQQKLNNTPLQYLRSGNSNGWSVDEFREVVIKMYNDKKISLKKLQNLLCEKDITKQDVRKEFIERNLVDTRYASRVVLGLLKDYFKANNKNTKVFTIRGQITSQFRKSRNLVKSRDCFYHHIVDALIIAYFRKFNYINELMQYNFNYKGKIDEETGELIQYNDTLFSTKDDDFTIQLRKLNDLNKYSEDIKISHKVDRKPNRKFSDETIYSARKIDNDYMKISKYKDIYGEEGKKFAKVIKDNLQKEKDLDKFLMKKVDPETFNLLVSIVINTTCGEKENPFVKYCEENNIDFIRKKSKNGNGPIIRSIRYYDGKVNSHLDISQKYSNLPNDKKVVLLQISPYRTDFYLTKEGNYKFLTIRYANIRENHNGYYIPKEVYESLKKSKKINDSDEFLFSLYQNDYLRITTNDPTENVPMIYRFVGTNNDLANKVEVKPIYCNKIDKPQIMKTIGKKVTKIEKFDVDVLGNKIAKKRDKCLQLEL

SEQ ID NO: 32 - protein, 1071 aa, synthetic construct
VAKYILGLDMGITSVGWGLIDYDSGKIVDKGVRLFKEATAEDNLNRRTHRGSRRLKRRRQQRIIELKRLLKKENILTDDYTPLNNVYYLRCKGLNEKLTNAELVSVLLHIAKIRGSSLEVVEDDEKKANEDKSSKANLSVNETKLRTSKEEICEKQYKDLIENGKVRGINNIYKSSSYESELKKILDNQGLSKEVNDKIINIIFRRRDFNEGPGSIESPTIYGRFIPHENGEIEIIPSMIEKMQGKCSIYQDEPRAPKMSFTADLFNFLNDLNNLEIQRADNYDNYITPDEKKLIINEYIIKKGKITIQKLCDVLQVKKENITGFRVNKGEPILTEFKGYQKVLKALNRIIDTEIVDAVIDVLTKYKDVEKRKEEIKKINNAEFSEEDIVKLSEITGIQQYHSLSYKAMKVIIPELYENPENQMEVITRLNLVKTSIDDLKGRTTIPSYSDRIYSPVAKRVHNETIKVVNAVIKKYGDLDSIVIEMARDRNTAEERKNIEQAQKRNQENREKAKEILDANHSFSKLDSKLVQKIMLYNQQDGKSMYSGKPINLNVLISDPNAYEIDHIIPLSISLDDSIANKVLVYRSENQQKLNNTPLQYLRSGNSNGWSVDEFREVVIKMYNDKKISLKKLQNLLCEKDITKQDVRKEFIERNLVDTRYASRVVLGLLKDYFKANNKNTKVFTIRGQITSQFRKSRNLVKSRDCFYHHIVDALIIAYFRKFNYINELMQYNFNYKGKIDEETGELIQYNDTLFSTKDDDFTIQLRKLNDLNKYSEDIKISHKVDRKPNRKFSDETIYSARKIDNDYMKISKYKDIYGEEGKKFAKVIKDNLQKEKDLDKFLMKKVDPETFNLLVSIVINTTCGEKENPFVKYCEENNIDFIRKKSKNGNGPIIRSIRYYDGKVNSHLDISQKYSNLPNDKKVVLLQISPYRTDFYLTKEGNYKFLTIRYANIRENHNGYYIPKEVYESLKKSKKINDSDEFLFSLYQNDYLRITTNDPTENVPMIYRFVGTNNDLANKVEVKPIYCNKIDKPQIMKTIGKKVTKIEKFDVDVLGNKIAKKRDKCLQLEL

SEQ ID NO: 33 - protein, 1120 aa, synthetic construct
MIPNPLLGLDKRTADGSEFESPKKKRKVGSAKYILGLDMGITSVGWGLIDYDSGKIVDKGVRLFKEATAEDNLNRRTHRGSRRLKRRRQQRIIELKRLLKKENILTDDYTPLNNVYYLRCKGLNEKLTNAELVSVLLHIAKIRGSSLEVVEDDEKKANEDKSSKANLSVNETKLRTSKEEICEKQYKDLIENGKVRGINNIYKSSSYESELKKILDNQGLSKEVNDKIINIIFRRRDFNEGPGSIESPTIYGRFIPHENGEIEIIPSMIEKMQGKCSIYQDEPRAPKMSFTADLFNFLNDLNNLEIQRADNYDNYITPDEKKLIINEYIIKKGKITIQKLCDVLQVKKENITGFRVNKGEPILTEFKGYQKVLKALNRIIDTEIVDAVIDVLTKYKDVEKRKEEIKKINNAEFSEEDIVKLSEITGIQQYHSLSYKAMKVIIPELYENPENQMEVITRLNLVKTSIDDLKGRTTIPSYSDRIYSPVAKRVHNETIKVVNAVIKKYGDLDSIVIEMARDRNTAEERKNIEQAQKRNQENREKAKEILDANHSFSKLDSKLVQKIMLYNQQDGKSMYSGKPINLNVLISDPNAYEIDHIIPLSISLDDSIANKVLVYRSENQQKLNNTPLQYLRSGNSNGWSVDEFREVVIKMYNDKKISLKKLQNLLCEKDITKQDVRKEFIERNLVDTRYASRVVLGLLKDYFKANNKNTKVFTIRGQITSQFRKSRNLVKSRDCFYHHIVDALIIAYFRKFNYINELMQYNFNYKGKIDEETGELIQYNDTLFSTKDDDFTIQLRKLNDLNKYSEDIKISHKVDRKPNRKFSDETIYSARKIDNDYMKISKYKDIYGEEGKKFAKVIKDNLQKEKDLDKFLMKKVDPETFNLLVSIVINTTCGEKENPFVKYCEENNIDFIRKKSKNGNGPIIRSIRYYDGKVNSHLDISQKYSNLPNDKKVVLLQISPYRTDFYLTKEGNYKFLTIRYANIRENHNGYYIPKEVYESLKKSKKINDSDEFLFSLYQNDYLRITTNDPTENVPMIYRFVGTNNDLANKVEVKPIYCNKIDKPQIMKTIGKKVTKIEKFDVDVLGNKIAKKRDKCLQLELSRKRTADGSEFESPKKKRKV

SEQ ID NO: 34 - DNA, 3216 bp, synthetic construct
  source 1..3216: mol_type = other DNA
gtggcaaaatatatattaggactagatatgggaattacttcggtaggttggggcctaattgactatgattctgggaaaatagttgataaaggcgttagactttttaaagaagcaactgcggaagataatttaaacagacgtactcatcgtggttcaagaaggttaaaaagacgtagacaacaaagaataattgaattaaaacgattattaaaaaaagaaaacattcttacagatgattatactcctttaaataatgtttactatcttagatgtaaaggcttaaacgaaaaattaacaaatgctgaacttgtatcagtattattacatatagcaaaaataagaggaagttcactagaagtagttgaagatgatgaaaaaaaggctaatgaggataaaagtagtaaagccaatttatcagtaaatgaaacaaaattaagaacttcaaaagaagaaatttgtgaaaaacagtataaagatctaatcgaaaatggtaaagtgcgtggaataaataatatttataaaagttcttcatatgagtcagaactcaaaaaaattttagataatcaaggcttgtcaaaagaagttaatgataaaataataaatataatttttagaagacgtgactttaatgaaggaccaggaagcatagaatcgcctactatatatggtagatttataccacatgaaaatggtgagatagaaattatacctagtatgattgaaaaaatgcagggaaaatgttctatatatcaagacgagcctcgtgcaccgaaaatgtcatttacagctgatttgtttaatttcttaaacgatttaaacaatttagaaatacagcgtgctgataactatgataattatattaccccagacgaaaaaaaactaataataaatgagtatatcatcaaaaaaggtaaaattacaattcaaaaattatgtgatgtactacaggtaaaaaaagaaaatattactggctttagagttaacaaaggtgaaccaatactcactgaatttaaaggctatcaaaaagtattaaaagctttaaatagaataattgatactgaaattgttgatgcggttattgacgttttaacaaaatataaagatgtagaaaaaagaaaagaagaaattaaaaaaataaacaatgcagaatttagtgaagaagacattgtaaaattatcagaaataactggtatacaacaatatcattcattgtcttataaagcaatgaaagtaataatacctgaattatatgaaaatcctgaaaatcaaatggaagtaattacaagattaaatctagtgaaaacttcaatagatgatttgaaaggaagaacaacaataccttcatatagcgacagaatatattcccctgtagctaaacgtgtacataatgagacaatcaaagtagtaaatgcggtaataaaaaaatatggagatttagatagtattgttatagaaatggcaagggatagaaatactgctgaagaaaggaaaaatattgaacaagcccaaaaacgaaatcaagaaaatagagaaaaagcaaaagagatactagatgctaatcatagtttttcgaaacttgattcaaaattagttcaaaagatcatgctttataatcaacaagatggtaaaagtatgtatagtggaaaaccaataaacttaaatgttttaataagtgaccctaatgcatacgagatagatcatatcataccactatcaatttcgcttgatgattcaattgccaataaagttctggtgtatagaagtgaaaaccaacaaaaattaaataatacaccacttcaatacttacgtagtggtaattcaaatggttggagcgtcgacgaattccgtgaagttgtaataaaaatgtataatgataaaaaaatatcactaaaaaaattacaaaacttattatgtgaaaaagatataacaaaacaagatgtaagaaaagaatttattgaaagaaatttagttgacacaagatatgcaagtagggtagttttgggacttctaaaagattatttcaaagccaacaataaaaatactaaagtatttacaataagaggccaaataacatcacaattcagaaaaagtagaaacttggtgaaaagtagagattgcttttatcatcatatagtagatgcattaataattgcatattttagaaaattcaattacattaatgaattaatgcaatacaattttaattataaagggaaaattgatgaagaaacaggagaattaatccaatataatgatacgttgttttcaacgaaggatgatgatttcacaatacaattaagaaaactaaatgaccttaataaatatagtgaagatatcaaaatttcgcataaagttgatagaaagcctaatagaaagtttagtgacgaaacaatctattctgcgagaaaaatcgataacgattatatgaaaatttcaaaatataaagatatttatggtgaagaaggtaaaaaatttgcaaaagtaattaaagataatcttcaaaaagaaaaagacttagataaattcttaatgaagaaagtagatcccgaaacttttaatttattagttagcattgttataaatacaacatgtggtgaaaaagaaaacccttttgttaaatattgtgaagaaaacaatatagattttattagaaaaaaatctaaaaatggtaatggtccgattattaggtctattagatattatgatggtaaagtaaatagtcaccttgatatctcacaaaaatattcaaacttaccaaatgacaaaaaggttgtacttttacaaatttcaccatatagaactgatttttatttaactaaagaaggaaactataaatttctaacaattagatatgctaatatcagagaaaaccataatggatattatataccaaaagaagtttacgaatcattaaagaaatcaaagaaaataaatgatagtgatgaattcttattttcgctatatcaaaatgattacctaagaattacaacaaatgatcctactgaaaatgttcctatgatttatcgatttgtaggtacaaataatgatttagcaaacaaagttgaagtaaaaccaatttattgtaataagatagataaaccacaaattatgaaaaccataggtaaaaaagtaactaaaattgaaaagtttgatgttgatgtactaggtaataaaattgctaaaaaaagagataaatgcttgcaattagagctgtag

SEQ ID NO: 35 - DNA, 3210 bp, synthetic construct
  source 1..3210: mol_type = other DNA
gccaagtacatcctgggcctggacatgggcatcaccagcgtgggctggggcctgattgattacgacagcggcaagatcgtggacaagggcgtgcggctgtttaaggaggccaccgccgaggacaacctgaacagaaggacccacagaggcagcagacggctgaagcggcggagacagcagagaatcatcgaactaaagcggctgctgaagaaggagaacatcctgaccgacgattacacccctctgaataacgtgtactacctgcggtgcaaaggcctgaacgaaaagctgaccaacgccgaactcgtgtctgttctgctgcacatcgccaagattcggggctcttcccttgaggtggtagaggacgatgagaagaaagcaaacgaggataaaagctccaaggccaatctgagcgtgaatgaaaccaagctgcggacaagcaaggaagaaatctgcgagaagcagtacaaggacctgatcgaaaacggcaaggtgcggggaatcaataacatctacaagtcctctagctacgaatctgaactgaagaagattctggacaatcagggcctgtccaaggaagtgaacgacaaaatcataaacattatcttcagacggagagacttcaacgagggccctggcagcattgagagccctaccatctatggaagattcatcccccacgagaacggcgagatcgagataatccccagcatgatcgagaaaatgcagggaaaatgcagcatctaccaggatgagcctcgggcccctaagatgagcttcaccgctgatttgtttaacttcctgaacgatctgaataacctggaaatccagagagccgacaactacgacaattacatcactccagacgagaagaagctgatcatcaacgagtacatcatcaagaagggcaagatcaccattcagaaactgtgcgacgtgctgcaagtgaagaaggagaacatcacaggcttccgcgtgaacaagggagaacccattctgaccgagttcaagggctatcagaaagtgctgaaggctctgaacagaatcatcgacaccgagatcgtggatgccgtgatcgacgtgctgacaaagtacaaggatgtggaaaaaagaaaagaggagatcaagaagattaacaacgctgagttcagcgaggaggacatcgtgaaactgagcgagatcacaggaatccagcagtaccacagcctgagctacaaggctatgaaagtgatcatcccagagctgtacgagaaccctgagaatcagatggaagtgatcacaagactgaatctggtgaaaaccagtatcgatgacctgaaaggcagaaccaccatccctagctacagcgaccggatctacagccctgtggccaagagagtgcataacgaaacaatcaaagtggtgaacgccgtgatcaagaaatacggagatctggacagcatcgtgatcgagatggccagagatagaaataccgccgaggagagaaagaacatcgagcaggcccagaaaagaaaccaggagaacagagaaaaagccaaggaaatcctggatgctaaccactctttcagcaaattggactctaaactggtccagaaaatcatgctgtacaaccagcaagacggcaagagcatgtacagcggcaaacctatcaatctgaacgtgctgatcagcgatcccaacgcttacgagatcgatcacatcatcccactgtccatttccctggacgacagcatcgccaacaaagttctggtgtacagaagcgaaaatcaacagaagttgaataatacccctctgcagtacctgagatctggcaacagcaacggctggagcgttgacgagttccgggaagtcgtcatcaagatgtacaacgacaagaagatctccctcaagaagctgcagaacctgctgtgtgaaaaggacatcacaaagcaagacgtgcggaaggaatttatcgagcggaatctggtggacacaagatacgccagcagggtggttctgggtctgctgaaggactacttcaaggccaacaacaagaacaccaaggtgttcaccatccggggccagatcaccagccagttcagaaagagcagaaacctggtgaagagcagagattgcttctaccaccacatcgtggacgccctgatcatcgcctacttcagaaagttcaactacatcaacgagctgatgcagtacaacttcaattataagggcaagatcgatgaagaaaccggcgagctgatccagtacaacgacaccctgttctctacaaaggacgacgattttacaatccagctgagaaagctgaatgacttgaacaagtacagcgaggacatcaaaatctctcacaaggtggacagaaagcctaaccggaagttcagcgatgagactatctacagcgccagaaaaattgataacgactacatgaagatctctaagtataaggatatctacggcgaagaaggcaagaagtttgccaaggtgatcaaagataatctgcagaaggaaaaggacctggataagttcctgatgaagaaagtggaccctgaaaccttcaacctgctcgtgtccatcgtgatcaacaccacctgtggcgagaaggagaatcctttcgtgaagtactgtgaagagaacaatatcgactttatccgaaaaaaaagtaaaaacggcaacggccccatcatccggtctatcagatactatgacggcaaggtcaacagccacctggacatctcacagaagtattctaacctgcctaacgacaaaaaggtggtgctgctgcaaatctccccgtacagaacagatttttacctgacaaaggagggaaattacaagttcctgaccatcagatacgccaacatcagagagaaccataatggatactacatccccaaggaagtgtacgagagcctgaagaaaagcaagaagatcaacgattcagacgagtttctgttctctctgtaccaaaacgactacctgagaatcacaacaaacgaccccaccgagaacgtgcctatgatctatagattcgtgggtacaaacaacgatctggcgaacaaggttgaggtgaagcctatctattgcaacaagatcgacaagcctcagatcatgaaaaccatagggaagaaggtgaccaagatagagaaattcgacgtagacgtgcttggcaacaagatcgccaaaaagcgggacaagtgcctgcagctggagctg

SEQ ID NO: 36 - DNA, 3363 bp, synthetic construct
  source 1..3363: mol_type = other DNA
atgatccccaatcccctgctgggcctggacaagcggactgccgacggcagtgagttcgagtctcccaagaaaaagaggaaagtgggatccgccaagtacatcctgggcctggacatgggcatcaccagcgtgggctggggcctgattgattacgacagcggcaagatcgtggacaagggcgtgcggctgtttaaggaggccaccgccgaggacaacctgaacagaaggacccacagaggcagcagacggctgaagcggcggagacagcagagaatcatcgaactaaagcggctgctgaagaaggagaacatcctgaccgacgattacacccctctgaataacgtgtactacctgcggtgcaaaggcctgaacgaaaagctgaccaacgccgaactcgtgtctgttctgctgcacatcgccaagattcggggctcttcccttgaggtggtagaggacgatgagaagaaagcaaacgaggataaaagctccaaggccaatctgagcgtgaatgaaaccaagctgcggacaagcaaggaagaaatctgcgagaagcagtacaaggacctgatcgaaaacggcaaggtgcggggaatcaataacatctacaagtcctctagctacgaatctgaactgaagaagattctggacaatcagggcctgtccaaggaagtgaacgacaaaatcataaacattatcttcagacggagagacttcaacgagggccctggcagcattgagagccctaccatctatggaagattcatcccccacgagaacggcgagatcgagataatccccagcatgatcgagaaaatgcagggaaaatgcagcatctaccaggatgagcctcgggcccctaagatgagcttcaccgctgatttgtttaacttcctgaacgatctgaataacctggaaatccagagagccgacaactacgacaattacatcactccagacgagaagaagctgatcatcaacgagtacatcatcaagaagggcaagatcaccattcagaaactgtgcgacgtgctgcaagtgaagaaggagaacatcacaggcttccgcgtgaacaagggagaacccattctgaccgagttcaagggctatcagaaagtgctgaaggctctgaacagaatcatcgacaccgagatcgtggatgccgtgatcgacgtgctgacaaagtacaaggatgtggaaaaaagaaaagaggagatcaagaagattaacaacgctgagttcagcgaggaggacatcgtgaaactgagcgagatcacaggaatccagcagtaccacagcctgagctacaaggctatgaaagtgatcatcccagagctgtacgagaaccctgagaatcagatggaagtgatcacaagactgaatctggtgaaaaccagtatcgatgacctgaaaggcagaaccaccatccctagctacagcgaccggatctacagccctgtggccaagagagtgcataacgaaacaatcaaagtggtgaacgccgtgatcaagaaatacggagatctggacagcatcgtgatcgagatggccagagatagaaataccgccgaggagagaaagaacatcgagcaggcccagaaaagaaaccaggagaacagagaaaaagccaaggaaatcctggatgctaaccactctttcagcaaattggactctaaactggtccagaaaatcatgctgtacaaccagcaagacggcaagagcatgtacagcggcaaacctatcaatctgaacgtgctgatcagcgatcccaacgcttacgagatcgatcacatcatcccactgtccatttccctggacgacagcatcgccaacaaagttctggtgtacagaagcgaaaatcaacagaagttgaataatacccctctgcagtacctgagatctggcaacagcaacggctggagcgttgacgagttccgggaagtcgtcatcaagatgtacaacgacaagaagatctccctcaagaagctgcagaacctgctgtgtgaaaaggacatcacaaagcaagacgtgcggaaggaatttatcgagcggaatctggtggacacaagatacgccagcagggtggttctgggtctgctgaaggactacttcaaggccaacaacaagaacaccaaggtgttcaccatccggggccagatcaccagccagttcagaaagagcagaaacctggtgaagagcagagattgcttctaccaccacatcgtggacgccctgatcatcgcctacttcagaaagttcaactacatcaacgagctgatgcagtacaacttcaattataagggcaagatcgatgaagaaaccggcgagctgatccagtacaacgacaccctgttctctacaaaggacgacgattttacaatccagctgagaaagctgaatgacttgaacaagtacagcgaggacatcaaaatctctcacaaggtggacagaaagcctaaccggaagttcagcgatgagactatctacagcgccagaaaaattgataacgactacatgaagatctctaagtataaggatatctacggcgaagaaggcaagaagtttgccaaggtgatcaaagataatctgcagaaggaaaaggacctggataagttcctgatgaagaaagtggaccctgaaaccttcaacctgctcgtgtccatcgtgatcaacaccacctgtggcgagaaggagaatcctttcgtgaagtactgtgaagagaacaatatcgactttatccgaaaaaaaagtaaaaacggcaacggccccatcatccggtctatcagatactatgacggcaaggtcaacagccacctggacatctcacagaagtattctaacctgcctaacgacaaaaaggtggtgctgctgcaaatctccccgtacagaacagatttttacctgacaaaggagggaaattacaagttcctgaccatcagatacgccaacatcagagagaaccataatggatactacatccccaaggaagtgtacgagagcctgaagaaaagcaagaagatcaacgattcagacgagtttctgttctctctgtaccaaaacgactacctgagaatcacaacaaacgaccccaccgagaacgtgcctatgatctatagattcgtgggtacaaacaacgatctggcgaacaaggttgaggtgaagcctatctattgcaacaagatcgacaagcctcagatcatgaaaaccatagggaagaaggtgaccaagatagagaaattcgacgtagacgtgcttggcaacaagatcgccaaaaagcgggacaagtgcctgcagctggagctgtctagaaagcggacagcagacggctccgaatttgaaagccctaagaaaaagagaaaggtgtga

SEQ ID NO: 37 - protein, 1068 aa, synthetic construct
AKYILGLDMGITSVGWGLIDYDSGKIVDKGVRLFKEATAEDNLKRRTHRGSRRLKRRRQQRIIELKRLLKKENILTDDYTPLNNVYSLRCKGLNEKLTNAELVSVLLHIAKIRGSSLEVVEDDEKKANEDKSSKANLSVNETKLRTSKEEICEKQYKDLIENGKVRGINNIYKSSSYESELKKILANQGLSKEVNDKIINIIFRRRDFNEGPGSMESPTIYGRFIPHKNGEVEVIHSMIEKMQGKCSIYQNEPRAAKMSFTADLFNFLNDLNNLEIQRADNYDNYITPDEKKLIINEYIIKKGKITIQKLCDVLQVKKENITGFRVNKGEPILTEFKGYQKVLKALNRIIDTEIVDAVIDVLTMHKDVEKRKEEIKKINNTEFSEEDIVKLSEITGIQQYHSLSYKAMKVIIPELYENPENQMEVITRLNLVKTSIDDLKGRTTIPSYSDRIYSPVAKRVHNETIKVVNAVIKKYGDLDSIVIEMARDRNTAEEQKNIEQVKKRNQENREKAKEIIEVNHSFSKLDSKLVQKIMLYNQQDGKSMYSGKPINLNVLISDREAYEIDHIIPLSISLDDSIANKVLVYKSENQQKLNNTPLQYLRSGNSNGWSVDEFREVVIKMYNDKKISLKKLQNLLCEKDITKYDVRKGFIERNLVDTRYASRVVLGLLKDYFKANNKNTKVFTVRGQITSQFRRAKNLKKDRNYFYHHIVDALIIAHFRKFNYISELMQYGFNYKEKIDEETGELIQYNDTLFPKKDDDFTIQLRKLNDLNIYSENIKISHKVDRKPNRQFSDETIYSARKIDNDYMKILKYKDIYGEEGKKFAKKIKDNLQKENELDIFLMKKVDPETFNLLVSIVKNTICGEKENPFVKYCEENNIDFIRKKSKNGNGPIIRSIRYYKKIESDYLDVSNKYINLPYNKKVLLDSTKNFRIDFYKSKNGIYKFLVIKYYYIDKINNKYYIPLNIYEKLKKDRCINDSDEFLFSVYKNEYLRITTKDSKKNVPNIYRFIGTTHNTIELKPLYCDEEQEKKRNRPTIGKKITKIEKFDVDVLGNKIAKKRDKCLQLEL

SEQ ID NO: 38 - protein, 1069 aa, synthetic construct
VAKYILGLDMGITSVGWGLIDYDSGKIVDKGVRLFKEATAEDNLKRRTHRGSRRLKRRRQQRIIELKRLLKKENILTDDYTPLNNVYSLRCKGLNEKLTNAELVSVLLHIAKIRGSSLEVVEDDEKKANEDKSSKANLSVNETKLRTSKEEICEKQYKDLIENGKVRGINNIYKSSSYESELKKILANQGLSKEVNDKIINIIFRRRDFNEGPGSMESPTIYGRFIPHKNGEVEVIHSMIEKMQGKCSIYQNEPRAAKMSFTADLFNFLNDLNNLEIQRADNYDNYITPDEKKLIINEYIIKKGKITIQKLCDVLQVKKENITGFRVNKGEPILTEFKGYQKVLKALNRIIDTEIVDAVIDVLTMHKDVEKRKEEIKKINNTEFSEEDIVKLSEITGIQQYHSLSYKAMKVIIPELYENPENQMEVITRLNLVKTSIDDLKGRTTIPSYSDRIYSPVAKRVHNETIKVVNAVIKKYGDLDSIVIEMARDRNTAEEQKNIEQVKKRNQENREKAKEIIEVNHSFSKLDSKLVQKIMLYNQQDGKSMYSGKPINLNVLISDREAYEIDHIIPLSISLDDSIANKVLVYKSENQQKLNNTPLQYLRSGNSNGWSVDEFREVVIKMYNDKKISLKKLQNLLCEKDITKYDVRKGFIERNLVDTRYASRVVLGLLKDYFKANNKNTKVFTVRGQITSQFRRAKNLKKDRNYFYHHIVDALIIAHFRKFNYISELMQYGFNYKEKIDEETGELIQYNDTLFPKKDDDFTIQLRKLNDLNIYSENIKISHKVDRKPNRQFSDETIYSARKIDNDYMKILKYKDIYGEEGKKFAKKIKDNLQKENELDIFLMKKVDPETFNLLVSIVKNTICGEKENPFVKYCEENNIDFIRKKSKNGNGPIIRSIRYYKKIESDYLDVSNKYINLPYNKKVLLDSTKNFRIDFYKSKNGIYKFLVIKYYYIDKINNKYYIPLNIYEKLKKDRCINDSDEFLFSVYKNEYLRITTKDSKKNVPNIYRFIGTTHNTIELKPLYCDEEQEKKRNRPTIGKKITKIEKFDVDVLGNKIAKKRDKCLQLEL

SEQ ID NO: 39 - protein, 1118 aa, synthetic construct
MIPNPLLGLDKRTADGSEFESPKKKRKVGSAKYILGLDMGITSVGWGLIDYDSGKIVDKGVRLFKEATAEDNLKRRTHRGSRRLKRRRQQRIIELKRLLKKENILTDDYTPLNNVYSLRCKGLNEKLTNAELVSVLLHIAKIRGSSLEVVEDDEKKANEDKSSKANLSVNETKLRTSKEEICEKQYKDLIENGKVRGINNIYKSSSYESELKKILANQGLSKEVNDKIINIIFRRRDFNEGPGSMESPTIYGRFIPHKNGEVEVIHSMIEKMQGKCSIYQNEPRAAKMSFTADLFNFLNDLNNLEIQRADNYDNYITPDEKKLIINEYIIKKGKITIQKLCDVLQVKKENITGFRVNKGEPILTEFKGYQKVLKALNRIIDTEIVDAVIDVLTMHKDVEKRKEEIKKINNTEFSEEDIVKLSEITGIQQYHSLSYKAMKVIIPELYENPENQMEVITRLNLVKTSIDDLKGRTTIPSYSDRIYSPVAKRVHNETIKVVNAVIKKYGDLDSIVIEMARDRNTAEEQKNIEQVKKRNQENREKAKEIIEVNHSFSKLDSKLVQKIMLYNQQDGKSMYSGKPINLNVLISDREAYEIDHIIPLSISLDDSIANKVLVYKSENQQKLNNTPLQYLRSGNSNGWSVDEFREVVIKMYNDKKISLKKLQNLLCEKDITKYDVRKGFIERNLVDTRYASRVVLGLLKDYFKANNKNTKVFTVRGQITSQFRRAKNLKKDRNYFYHHIVDALIIAHFRKFNYISELMQYGFNYKEKIDEETGELIQYNDTLFPKKDDDFTIQLRKLNDLNIYSENIKISHKVDRKPNRQFSDETIYSARKIDNDYMKILKYKDIYGEEGKKFAKKIKDNLQKENELDIFLMKKVDPETFNLLVSIVKNTICGEKENPFVKYCEENNIDFIRKKSKNGNGPIIRSIRYYKKIESDYLDVSNKYINLPYNKKVLLDSTKNFRIDFYKSKNGIYKFLVIKYYYIDKINNKYYIPLNIYEKLKKDRCINDSDEFLFSVYKNEYLRITTKDSKKNVPNIYRFIGTTHNTIELKPLYCDEEQEKKRNRPTIGKKITKIEKFDVDVLGNKIAKKRDKCLQLELSRKRTADGSEFESPKKKRKV

SEQ ID NO: 40 - DNA, 3210 bp, synthetic construct
  source 1..3210: mol_type = other DNA
gtggcaaaatatatattaggactagatatgggaattacttcggtaggttggggcctaattgactatgattctggaaaaatagttgataaaggcgttagactttttaaagaagcaactgcggaagacaatttaaaaagacgtacacatcgtggatcaagaaggttaaaaagacgtagacaacaaagaataattgaattaaaacgattattgaaaaaagaaaacatccttacagatgattatactcctttaaataatgtttactctcttagatgtaaaggtttaaatgaaaaattaacaaatgccgaacttgtatcagtattattacatatagcaaaaataagaggaagttccctagaagtagttgaagatgatgaaaaaaaggctaatgaggataaaagtagtaaagccaatttatcagtaaatgaaacaaaattaagaacttcaaaagaagaaatttgtgaaaaacaatataaagatctaatcgaaaatggtaaagttcgtggaataaataatatttataaaagttcttcatatgagtcagaactcaaaaaaattttagctaatcaaggcttgtcaaaagaagttaatgataagataataaatataattttcagaagacgtgactttaatgaaggaccaggaagcatggaatcgcctactatatatggtagatttataccacataaaaatggtgaggtagaagttatacatagtatgattgaaaaaatgcaagggaaatgttccatataccaaaacgaacctcgtgcagcaaaaatgtcatttacagctgatttgtttaattttttaaatgatttaaacaatttagaaatacagcgtgctgataactatgataattatattaccccagacgaaaaaaagctaataataaatgagtatatcataaaaaaaggtaaaattacaattcaaaaattatgcgatgtactacaggtaaaaaaagaaaatattactggctttagagttaacaaaggtgaaccaatactcactgaatttaaaggttatcaaaaagtattaaaagctttaaatagaataattgatactgaaattgttgatgcggttattgatgttttaacaatgcataaagatgtagaaaaaagaaaagaagaaattaaaaaaataaacaatacagagtttagcgaagaagacattgtaaaattatcagaaataactggtatacaacagtatcattcattgtcttataaagcaatgaaagtaataatacctgaattatatgaaaatcctgaaaatcaaatggaagtaattacaagattaaatctagtgaaaacttcaatagatgatttgaaaggaagaacaacaataccttcatatagcgacagaatatattcccctgtagctaaacgtgtacataatgagacaatcaaagtagtaaatgcggtaataaaaaaatatggagatttagatagtattgttatagaaatggcaagggatagaaatactgctgaagaacaaaaaaatattgaacaagtcaaaaagcgaaatcaagaaaatagagaaaaagcaaaagaaataatagaagttaatcatagtttttcaaaacttgattcaaaattagttcaaaagattatgctttataatcaacaagatggtaaaagtatgtatagtggaaaaccaataaacttaaatgttttaataagtgaccgtgaagcatacgagatagatcatatcataccactatcaatttcacttgatgattcaattgccaataaagttttggtatataaaagtgaaaaccaacaaaaattaaataatacaccacttcaatatttacgtagtggtaactcaaatggctggagcgtcgacgaattccgtgaagttgtaataaaaatgtataatgataaaaaaatatcactaaaaaaattacaaaacttgttatgtgaaaaagatataacaaaatatgatgtaagaaaaggatttattgaaagaaatttagttgacacaagatatgcaagtagagtagttttgggacttctaaaagactatttcaaagctaacaataaaaatactaaagtatttacagtaagaggtcaaatcacatcacagttcagaagagccaaaaacttgaaaaaagatagaaattacttttaccatcatatagtggatgcattaataattgcacattttaggaaattcaattacattagtgaattaatgcaatatggctttaattataaagaaaaaatcgatgaagaaacaggagaattaattcaatataatgatacgttgtttccaaagaaggatgatgatttcacaatacaattaagaaaactaaatgatcttaatatatatagtgaaaacatcaaaatttcgcataaagttgatagaaagcctaatagacagtttagtgatgaaacaatctattctgcgagaaaaatcgacaacgattatatgaaaattttgaaatataaagatatttatggtgaagaaggcaaaaaatttgcaaaaaaaattaaagataatcttcaaaaggaaaatgaattagatatattcttaatgaaaaaagttgatccagaaactttcaatttattagttagcattgttaagaatacaatatgtggtgaaaaagaaaatccatttgttaaatactgtgaagaaaacaatatagattttattagaaaaaaatctaaaaatggcaatggtccgattattagatctattagatattataaaaaaatagagagtgactatttggatgtttcaaataaatatattaatttaccatataacaaaaaggttttgctagatagcactaaaaattttagaatagatttttataaatctaaaaatggcatttataaatttttggtaataaagtattactatatagataaaatcaataataaatattacattccattaaatatatatgaaaaattaaaaaaagatagatgtattaatgatagtgatgagttcttattttcagtatataagaacgagtatttaagaattacaaccaaagatagtaagaaaaatgttccaaatatttatagatttattggaacaacacataatacaattgaattaaaaccgctttattgtgacgaagaacaagaaaaaaaacgtaacagaccaaccataggtaaaaaaataactaaaattgaaaagtttgatgttgatgtactaggtaataaaattgctaaaaaaagagataaatgcttgcaattagagctgtag

SEQ ID NO: 41 - DNA, 3204 bp, synthetic construct
  source 1..3204: mol_type = other DNA
gctaagtacatcctgggcctggatatgggcatcacatccgtgggctggggcctgatcgattacgatagcggcaaaatcgtggataagggcgtgcggctgttcaaggaggccaccgccgaggataacctcaagaggaggacccatagaggaagcagacggctgaagcggcggagacagcaaagaatcatcgaactgaagagactgctgaagaaggaaaacatcctgacagacgattatacacctctgaacaacgtctacagcctgagatgcaaggggctgaatgaaaagctcacaaacgctgagctggtgtccgtgctgctgcacatcgccaaaatccggggaagcagccttgaagtggtggaagatgatgagaagaaggctaatgaggacaagtctagcaaagcaaacctgagcgtgaatgaaacaaagctcagaaccagcaaggaagaaatctgcgaaaaacagtacaaggacctgattgaaaacggcaaagttagaggcatcaacaacatctacaagagctccagctacgaaagcgagctgaagaaaattctcgccaaccagggcctgtccaaggaggtgaacgacaagatcatcaacatcatcttcagacggcgggacttcaacgagggccccggcagtatggaatctccaacaatctacggccgatttatcccccacaagaacggcgaggtggaagtgatccatagcatgatcgaaaagatgcagggcaagtgcagcatctaccagaacgaacctagagccgcaaagatgagcttcaccgccgacctcttcaacttcctgaacgaccttaacaacctggagatccagagagccgataattatgacaactacatcacacctgatgagaagaagctgatcataaacgagtacatcatcaagaagggcaagattacgattcagaagctgtgcgacgtgctgcaggtaaaaaaggagaacatcaccggcttccgcgtcaacaaaggagagcccatcctgaccgagttcaagggttatcagaaggtgctgaaggctctgaacagaatcatcgacaccgagatcgtggatgccgtgatcgatgtgctgaccatgcacaaggatgtggaaaagcggaaagaagaaatcaagaagattaacaatactgagttcagcgaagaggacatcgtgaaactgtctgagatcacaggcatccagcaataccacagcctgagctacaaggccatgaaggtcatcatcccagagctgtacgagaatcctgaaaaccagatggaagtgatcaccagactgaacctggtgaaaacctctatcgatgacctgaaaggaagaacaaccatccccagctacagcgacagaatctacagccctgttgctaaaagagtgcacaatgaaaccatcaaggtggttaatgccgtgattaagaagtacggcgacctggatagcatcgttatcgagatggccagagataggaacacagctgaagagcagaagaacatcgaacaggtgaagaagagaaaccaggagaatagagagaaggccaaggaaatcatcgaagtcaaccacagtttcagcaaactggattctaagctggtgcagaaaatcatgctgtacaaccagcaggacggcaaatctatgtactctggcaagcccatcaacctgaacgtgctgatcagcgacagagaggcctacgagatcgaccacatcatccctctatcaatctctctggatgacagcatcgccaataaagtgctggtctacaaatctgagaaccagcaaaagctgaataacacccctctgcagtacctgagaagcggcaacagcaacggctggagcgtggacgagttcagagaagtggtcatcaagatgtacaacgacaagaagatctccctgaagaagctgcagaatctcctgtgtgagaaggacataaccaagtacgacgtgcggaagggattcatcgagagaaatctggtcgacacccggtacgccagcagagtggtgctgggactgctgaaggactactttaaggccaacaataaaaacacaaaagtgttcacagtgcgcggccagatcaccagccagttcagacgggccaagaacctgaaaaaggacagaaactacttttaccaccacatcgtggacgccctgatcatcgcccacttcagaaagtttaactacatctccgagctgatgcaatacggatttaactacaaggagaagattgacgaggaaaccggcgagctgatccagtacaatgacacactgttccctaagaaagacgatgattttaccatccagctgcggaagctgaacgatctgaacatctacagcgaaaacatcaagatcagccacaaagtggacagaaaacctaatagacaattctctgatgaaaccatctatagcgcccggaagatcgacaatgactacatgaaaatcctgaagtacaaagatatctacggcgaggaaggcaagaagttcgccaaaaagatcaaagataacctgcagaaggaaaatgagctggacatcttccttatgaagaaggtggaccctgagacattcaacctgctggtttccatcgtgaagaataccatctgtggcgaaaaagagaaccccttcgtgaaatactgcgaagagaacaacatcgacttcatcagaaagaaaagcaagaacggcaatggccctatcatccggagcatcagatactacaaaaagatcgagagcgattacctggacgtgtccaacaagtacatcaatctgccttacaacaagaaggtgctgctcgattccaccaagaacttccggatcgacttctacaagagcaagaatggcatttacaagttcctggtgatcaagtattattacatcgacaagatcaacaacaaatattacatcccactgaacatctatgagaagctgaagaaagaccggtgcatcaacgacagcgacgagtttctgtttagcgtgtacaaaaacgaatacctgcggatcacaacaaaggacagcaagaaaaacgtgcctaacatataccggttcatcggcaccacccacaacaccatcgagctgaagcctctgtactgtgacgaggaacaggagaagaagcggaaccgccccaccatcggcaagaagattaccaagatcgagaagttcgacgtggacgtcctgggaaacaagatcgccaagaagagggacaagtgcctgcagctggaactg

SEQ ID NO: 42 - DNA, 3357 bp, synthetic construct
  source 1..3357: mol_type = other DNA
atgatccccaatcccctgctgggcctggacaagcggactgccgacggcagtgagttcgagtctcccaagaaaaagaggaaagtgggatccgctaagtacatcctgggcctggatatgggcatcacatccgtgggctggggcctgatcgattacgatagcggcaaaatcgtggataagggcgtgcggctgttcaaggaggccaccgccgaggataacctcaagaggaggacccatagaggaagcagacggctgaagcggcggagacagcaaagaatcatcgaactgaagagactgctgaagaaggaaaacatcctgacagacgattatacacctctgaacaacgtctacagcctgagatgcaaggggctgaatgaaaagctcacaaacgctgagctggtgtccgtgctgctgcacatcgccaaaatccggggaagcagccttgaagtggtggaagatgatgagaagaaggctaatgaggacaagtctagcaaagcaaacctgagcgtgaatgaaacaaagctcagaaccagcaaggaagaaatctgcgaaaaacagtacaaggacctgattgaaaacggcaaagttagaggcatcaacaacatctacaagagctccagctacgaaagcgagctgaagaaaattctcgccaaccagggcctgtccaaggaggtgaacgacaagatcatcaacatcatcttcagacggcgggacttcaacgagggccccggcagtatggaatctccaacaatctacggccgatttatcccccacaagaacggcgaggtggaagtgatccatagcatgatcgaaaagatgcagggcaagtgcagcatctaccagaacgaacctagagccgcaaagatgagcttcaccgccgacctcttcaacttcctgaacgaccttaacaacctggagatccagagagccgataattatgacaactacatcacacctgatgagaagaagctgatcataaacgagtacatcatcaagaagggcaagattacgattcagaagctgtgcgacgtgctgcaggtaaaaaaggagaacatcaccggcttccgcgtcaacaaaggagagcccatcctgaccgagttcaagggttatcagaaggtgctgaaggctctgaacagaatcatcgacaccgagatcgtggatgccgtgatcgatgtgctgaccatgcacaaggatgtggaaaagcggaaagaagaaatcaagaagattaacaatactgagttcagcgaagaggacatcgtgaaactgtctgagatcacaggcatccagcaataccacagcctgagctacaaggccatgaaggtcatcatcccagagctgtacgagaatcctgaaaaccagatggaagtgatcaccagactgaacctggtgaaaacctctatcgatgacctgaaaggaagaacaaccatccccagctacagcgacagaatctacagccctgttgctaaaagagtgcacaatgaaaccatcaaggtggttaatgccgtgattaagaagtacggcgacctggatagcatcgttatcgagatggccagagataggaacacagctgaagagcagaagaacatcgaacaggtgaagaagagaaaccaggagaatagagagaaggccaaggaaatcatcgaagtcaaccacagtttcagcaaactggattctaagctggtgcagaaaatcatgctgtacaaccagcaggacggcaaatctatgtactctggcaagcccatcaacctgaacgtgctgatcagcgacagagaggcctacgagatcgaccacatcatccctctatcaatctctctggatgacagcatcgccaataaagtgctggtctacaaatctgagaaccagcaaaagctgaataacacccctctgcagtacctgagaagcggcaacagcaacggctggagcgtggacgagttcagagaagtggtcatcaagatgtacaacgacaagaagatctccctgaagaagctgcagaatctcctgtgtgagaaggacataaccaagtacgacgtgcggaagggattcatcgagagaaatctggtcgacacccggtacgccagcagagtggtgctgggactgctgaaggactactttaaggccaacaataaaaacacaaaagtgttcacagtgcgcggccagatcaccagccagttcagacgggccaagaacctgaaaaaggacagaaactacttttaccaccacatcgtggacgccctgatcatcgcccacttcagaaagtttaactacatctccgagctgatgcaatacggatttaactacaaggagaagattgacgaggaaaccggcgagctgatccagtacaatgacacactgttccctaagaaagacgatgattttaccatccagctgcggaagctgaacgatctgaacatctacagcgaaaacatcaagatcagccacaaagtggacagaaaacctaatagacaattctctgatgaaaccatctatagcgcccggaagatcgacaatgactacatgaaaatcctgaagtacaaagatatctacggcgaggaaggcaagaagttcgccaaaaagatcaaagataacctgcagaaggaaaatgagctggacatcttccttatgaagaaggtggaccctgagacattcaacctgctggtttccatcgtgaagaataccatctgtggcgaaaaagagaaccccttcgtgaaatactgcgaagagaacaacatcgacttcatcagaaagaaaagcaagaacggcaatggccctatcatccggagcatcagatactacaaaaagatcgagagcgattacctggacgtgtccaacaagtacatcaatctgccttacaacaagaaggtgctgctcgattccaccaagaacttccggatcgacttctacaagagcaagaatggcatttacaagttcctggtgatcaagtattattacatcgacaagatcaacaacaaatattacatcccactgaacatctatgagaagctgaagaaagaccggtgcatcaacgacagcgacgagtttctgtttagcgtgtacaaaaacgaatacctgcggatcacaacaaaggacagcaagaaaaacgtgcctaacatataccggttcatcggcaccacccacaacaccatcgagctgaagcctctgtactgtgacgaggaacaggagaagaagcggaaccgccccaccatcggcaagaagattaccaagatcgagaagttcgacgtggacgtcctgggaaacaagatcgccaagaagagggacaagtgcctgcagctggaactgtctagaaagcggacagcagacggctccgaatttgaaagccctaagaaaaagagaaaggtgtga

SEQ ID NO: 43 - protein, 1005 aa, synthetic construct
VEVKTEAVGRLGRPRYLVLGMDPGIASCGFALLDINNHEILEMGVRLFDAPLHPKTKQSLAAIRRGFRSTRRNIDRTQDRLKHCFECFKKYGVVPDDADKEYLHTVKGDKQPLKLRVDGLNRMLTNREWAMALYSLCKRRGYIPHGEGAQDKSSDDGKVLAAISANEKEIAAGKFRTAGEWLASRPTSRNRGGNYEMCVKHEQLIEETRILFEAQRSFGSKVASEDFEREYIDICNWERPRDDFDRRTYGLVGSCVYFPQEKRAARCTLTSELVSAYGALGNITIVLGDGGVRRLTAAERDECIRILFTADPNAKTRVVKFGALRKKLDLSSRDYFKGVPADDEKTREVFKPSGWRALYSTLRESNPQLLARLRDDIDLADAVMEAAAYASSASSLENCLSQLPLSDDEKQALCALPYSSKVLNGYGSRSKKALDLLLACLKEPEVLNLTDAEAACGLDALRLGGPQLERSEKLIPYEAWVSAAGRTNNNPVVIRAMSQMRKVVNAICGKWGVPNEIHVELDRELRLPKRAKEEIAKANKKNEKERERIAVQAADILSCSPDEVSGKQIEKYRLWEEQENSDLYTGERIHVQRLLSDDTYTQIDHILPFSRTGENSRNNKVLVLSESNQQKREQTPYEWMNSGQPGAPSWDEFKLRVQANQHLKHRKKEFLLEQDLESKEVEFLQRNVTDTAYMSREVCAYLSDCLLFPDDGMKVHVVPTTGRATAWLRRRWGLNFGATGEKDRSDDRHHATDACVIAACSRGLVIKTAKVMKGTHWSITRNMTSEERRDARMEALRESMPWESFADDVRARRDFVVPTRYVPRKGSGELFEQTVYSYVGRNEKGQDLGRKKGVDKDKVMGNAVVSDDGKSVIKVSEMLCLRLWHDPEIKKKGKVVGGWYADPVYKSDIPALKDGTYVPRIAKAHCGRSAWAPVPERLLLQRPVELYLGDVVRVKEMVGCLSGFDMGGVAWKFVDPMTRESISAPTIGSLTGKDAPKVVRGGIVG

SEQ ID NO: 44 - protein, 1006 aa, synthetic construct
MVEVKTEAVGRLGRPRYLVLGMDPGIASCGFALLDINNHEILEMGVRLFDAPLHPKTKQSLAAIRRGFRSTRRNIDRTQDRLKHCFECFKKYGVVPDDADKEYLHTVKGDKQPLKLRVDGLNRMLTNREWAMALYSLCKRRGYIPHGEGAQDKSSDDGKVLAAISANEKEIAAGKFRTAGEWLASRPTSRNRGGNYEMCVKHEQLIEETRILFEAQRSFGSKVASEDFEREYIDICNWERPRDDFDRRTYGLVGSCVYFPQEKRAARCTLTSELVSAYGALGNITIVLGDGGVRRLTAAERDECIRILFTADPNAKTRVVKFGALRKKLDLSSRDYFKGVPADDEKTREVFKPSGWRALYSTLRESNPQLLARLRDDIDLADAVMEAAAYASSASSLENCLSQLPLSDDEKQALCALPYSSKVLNGYGSRSKKALDLLLACLKEPEVLNLTDAEAACGLDALRLGGPQLERSEKLIPYEAWVSAAGRTNNNPVVIRAMSQMRKVVNAICGKWGVPNEIHVELDRELRLPKRAKEEIAKANKKNEKERERIAVQAADILSCSPDEVSGKQIEKYRLWEEQENSDLYTGERIHVQRLLSDDTYTQIDHILPFSRTGENSRNNKVLVLSESNQQKREQTPYEWMNSGQPGAPSWDEFKLRVQANQHLKHRKKEFLLEQDLESKEVEFLQRNVTDTAYMSREVCAYLSDCLLFPDDGMKVHVVPTTGRATAWLRRRWGLNFGATGEKDRSDDRHHATDACVIAACSRGLVIKTAKVMKGTHWSITRNMTSEERRDARMEALRESMPWESFADDVRARRDFVVPTRYVPRKGSGELFEQTVYSYVGRNEKGQDLGRKKGVDKDKVMGNAVVSDDGKSVIKVSEMLCLRLWHDPEIKKKGKVVGGWYADPVYKSDIPALKDGTYVPRIAKAHCGRSAWAPVPERLLLQRPVELYLGDVVRVKEMVGCLSGFDMGGVAWKFVDPMTRESISAPTIGSLTGKDAPKVVRGGIVG

SEQ ID NO: 45 - protein, 1055 aa, synthetic construct
MIPNPLLGLDKRTADGSEFESPKKKRKVGSVEVKTEAVGRLGRPRYLVLGMDPGIASCGFALLDINNHEILEMGVRLFDAPLHPKTKQSLAAIRRGFRSTRRNIDRTQDRLKHCFECFKKYGVVPDDADKEYLHTVKGDKQPLKLRVDGLNRMLTNREWAMALYSLCKRRGYIPHGEGAQDKSSDDGKVLAAISANEKEIAAGKFRTAGEWLASRPTSRNRGGNYEMCVKHEQLIEETRILFEAQRSFGSKVASEDFEREYIDICNWERPRDDFDRRTYGLVGSCVYFPQEKRAARCTLTSELVSAYGALGNITIVLGDGGVRRLTAAERDECIRILFTADPNAKTRVVKFGALRKKLDLSSRDYFKGVPADDEKTREVFKPSGWRALYSTLRESNPQLLARLRDDIDLADAVMEAAAYASSASSLENCLSQLPLSDDEKQALCALPYSSKVLNGYGSRSKKALDLLLACLKEPEVLNLTDAEAACGLDALRLGGPQLERSEKLIPYEAWVSAAGRTNNNPVVIRAMSQMRKVVNAICGKWGVPNEIHVELDRELRLPKRAKEEIAKANKKNEKERERIAVQAADILSCSPDEVSGKQIEKYRLWEEQENSDLYTGERIHVQRLLSDDTYTQIDHILPFSRTGENSRNNKVLVLSESNQQKREQTPYEWMNSGQPGAPSWDEFKLRVQANQHLKHRKKEFLLEQDLESKEVEFLQRNVTDTAYMSREVCAYLSDCLLFPDDGMKVHVVPTTGRATAWLRRRWGLNFGATGEKDRSDDRHHATDACVIAACSRGLVIKTAKVMKGTHWSITRNMTSEERRDARMEALRESMPWESFADDVRARRDFVVPTRYVPRKGSGELFEQTVYSYVGRNEKGQDLGRKKGVDKDKVMGNAVVSDDGKSVIKVSEMLCLRLWHDPEIKKKGKVVGGWYADPVYKSDIPALKDGTYVPRIAKAHCGRSAWAPVPERLLLQRPVELYLGDVVRVKEMVGCLSGFDMGGVAWKFVDPMTRESISAPTIGSLTGKDAPKVVRGGIVGSRKRTADGSEFESPKKKRKV

SEQ ID NO: 46 - DNA, 3021 bp, synthetic construct
  source 1..3021: mol_type = other DNA
atggtcgaggttaaaacagaggcggtcggtcggctcggtcgtcctcgatatctggttttgggaatggacccaggcattgcgagttgcggcttcgcgttgctcgatatcaacaatcatgagatcttggaaatgggcgttcgcttgttcgatgccccgttgcatcctaaaacgaagcagagcttggccgctattcgtcgcggcttccgttcgacgcggcgcaatatcgaccgaacgcaggatcgactcaagcattgcttcgagtgctttaagaaatacggcgtcgttccagatgatgccgataaagaatatcttcatacggtcaaaggggataaacagcccctcaagcttcgtgtcgatggcctgaatcgcatgctcacgaacagggagtgggccatggccctctattctctgtgcaagcgtcgtggctacattccgcacggcgaaggcgctcaggataaatcgagcgatgacggcaaggtgcttgctgcgatcagcgcgaatgaaaaagaaatcgcagcaggaaaattcagaaccgcaggcgaatggctcgcgtcgcgtcccacgagcaggaatcgcgggggcaattacgagatgtgcgtcaagcacgagcagcttatcgaagaaacccgtattctgttcgaagcacagcgctcgttcggttccaaggttgcctctgaagatttcgagcgcgaatacatcgacatatgcaattgggaacggccgcgcgacgatttcgataggcgtacgtacggcctggtggggtcatgcgtttactttccgcaagagaagcgcgcggcacgttgcacgcttacgagcgagcttgtttccgcgtatggcgctttgggaaatataaccatcgtgctcggcgacggcggcgtgcgccgtcttacggcggccgaacgagacgaatgcattcgcatattgtttacggccgatccgaatgcgaagacccgcgttgtgaagttcggtgccttgcgcaaaaagctcgacttgagttcgcgcgattatttcaagggcgttcctgccgatgacgaaaagacccgtgaggtgttcaaacccagcggttggcgcgccttgtattcgacgttgcgggaatccaatccgcagcttctcgcaaggttgagggacgatatcgatttggcggatgccgttatggaggccgccgcgtatgcatcatctgcgtcgagcttggagaactgcctcagccagcttcctcttagcgatgatgaaaagcaggcgctttgcgcgctgccgtattcgtctaaagtgctgaatggatatggaagccgctcgaaaaaggcgctcgacttgctgcttgcttgtctcaaagaacccgaagtgctcaatctgaccgatgccgaggcggcatgtgggttggatgcgttgcgtcttggcggcccacagctcgagcgttccgagaagctgattccctatgaggcctgggtaagcgcggcgggtcgtacgaataacaatcctgtcgtcattcgtgctatgtcgcaaatgcgcaaggtcgtaaatgccatatgcggcaaatggggcgttccgaacgaaatccacgtcgaacttgaccgcgagcttcggttgcctaagcgtgcgaaagaagaaattgccaaggccaataagaaaaacgagaaagagcgcgagcgcatagccgttcaggccgcggacattctttcctgcagccccgatgaggtgagcggaaagcagatagagaagtaccgcttgtgggaggagcaggaaaactccgatttgtatacgggcgagcgtatccacgtccaacgccttttgagcgatgacacctatacgcagatcgaccatattcttccgttttcgcgaacgggagagaattcgcgtaataacaaggtgctcgtgctttcggaaagcaatcagcagaaacgcgagcagactccgtatgaatggatgaattcgggccagcctggtgctccgtcgtgggacgagttcaaacttcgagttcaggcgaaccagcatcttaagcaccgcaagaaagaattcttgctcgagcaggatttggaatcgaaggaagtcgaattcctccagcgaaacgtgaccgacacggcttatatgtcgcgcgaagtctgcgcctatctgtccgattgcctgttgttccccgatgacggcatgaaggttcacgttgttcccacgacgggtcgcgctacggcatggttgcgtcgccgatgggggctgaacttcggagcaacgggggagaaggaccgttcggacgaccgtcatcatgctacggacgcctgcgttattgccgcgtgcagcaggggccttgtcatcaagacggcaaaggttatgaagggaacgcactggagcatcacgcgtaatatgacgtcggaggagcgcagagatgcccgtatggaggcgttgcgtgagagcatgccctgggaaagcttcgccgacgatgtccgcgcacgtcgcgatttcgtggtacccacgcgctatgtgcctcgtaaaggcagcggtgagcttttcgagcagacagtgtattcgtatgtcggtcggaacgaaaaagggcaggaccttggccgaaagaagggcgtcgacaaagataaggtcatgggcaatgccgtagtgtccgatgatggaaaatcggtcattaaggtgagcgagatgctgtgcctgcgtttgtggcacgatccagagattaaaaagaaggggaaggtcgttggcggctggtatgccgacccggtgtacaagtctgatattcccgctcttaaggatggcacgtatgtgccgcgaatcgcaaaggcccactgcggccgttcggcgtgggcgcctgttcccgaacggcttttgctccagcgtccggttgagttgtatcttggtgatgttgtcagggtgaaagagatggttgggtgcctttctggttttgacatgggtggtgttgcatggaagttcgttgacccaatgacgcgtgagtcgatatctgcgccgaccattggaagtcttacgggcaaggatgctcccaaggtggttcgtgggggcattgtcggttag

SEQ ID NO: 47 - DNA, 3015 bp, synthetic construct
  source 1..3015: mol_type = other DNA
gtggaagtgaaaaccgaagccgtgggcagactgggccggcctagatatctggtcctgggcatggaccccggcatcgcctcctgcggctttgccctcctggacatcaacaatcacgagatcctggaaatgggagttcggctgtttgacgcacctctgcaccctaaaactaagcagagcctggccgccatccggaggggatttcggtccacccggagaaacatcgaccggacccaggacaggctcaaacattgttttgagtgcttcaagaaatacggcgtggttcctgacgatgccgacaaggagtacctgcacaccgtgaaaggagataagcaacctctgaagctgagagtggacggtctgaacagaatgctgaccaatagagagtgggctatggccctgtattctctgtgcaagcggagagggtacatcccccacggcgagggagctcaggacaaaagcagcgacgatggaaaagtgctcgctgcaattagcgctaacgagaaggagattgccgccggaaagttcagaaccgccggcgaatggctggccagccggcccacctccagaaatagaggcggcaactacgaaatgtgcgtgaagcacgagcaattgatcgaggaaaccagaatcctgttcgaggcccagcggagcttcggcagtaaggtcgcttctgaggacttcgagcgggagtacatcgacatctgcaactgggaacggcctcgggacgacttcgaccggcgcacctacggcctggtggggagctgcgtgtacttcccccaggagaaaagagccgctcggtgcaccctgacatccgagctggtgtctgcctatggagcactgggcaatatcacaatcgtgctgggcgatggcggcgtgcgccggctgaccgccgccgaacgggacgagtgcatcagaatcctgttcaccgccgatcctaacgccaagaccagagtggtgaagttcggcgccttacgaaaaaagctggacctgtccagcagagactacttcaagggcgtgcccgccgatgatgaaaagaccagagaagtctttaagccaagcggctggcgcgccctgtacagtacactgagagagagcaacccccagctgctggcccgattacgagatgatatcgatctcgccgatgctgtgatggaagccgctgcttacgccagcagcgccagctctctggaaaactgcctgagccagctgcctctgagcgacgacgagaagcaggccttgtgcgccctgccatactcaagcaaagtgctgaacggctacggcagcagatctaagaaggccctggaccttctgctggcctgtctcaaggagcctgaagttctcaacctgaccgatgctgaagctgcttgtggcctcgatgccctgagactgggcggtcctcagctggaacggagtgaaaagctgataccctacgaggcctgggtgagcgctgccggccgtaccaacaacaatcccgtggtgattagagccatgagccaaatgagaaaggtggtcaacgccatctgcggcaagtggggcgtgccaaatgagatccacgtggaactggatcgggaactgcggctgcccaagcgggctaaagaagagatcgccaaggccaacaagaagaacgagaaagagagagagagaatcgccgtgcaagccgccgacatactgagttgttctcctgatgaggtgtctggcaagcagatcgagaagtacagactgtgggaggaacaggagaatagcgacctgtacacaggcgagagaatccacgtccagagacttctgagcgacgatacctatacacagatcgaccacatcctgcccttcagccgcacaggcgagaacagcagaaacaacaaggtgctcgtgctgtccgaaagcaaccagcagaagagagagcagaccccttatgagtggatgaacagcggacagcctggcgctcccagctgggacgagttcaagctgcgggtgcaggccaaccagcacctgaagcataggaagaaagagttcctgctggagcaggatctggaatctaaggaagtggagttcctgcagagaaacgtgaccgacaccgcctacatgagccgggaagtgtgcgcctacctgagcgactgcctgctcttccctgacgacggcatgaaggtgcacgtggtgcccacaacaggcagagccacagcctggctgagaagaagatggggactgaacttcggcgctaccggcgagaaagacagaagcgatgaccggcaccacgccaccgacgcttgcgtgatcgccgcctgtagcagaggcctggtgatcaagacagccaaggtcatgaagggcacccactggagcatcaccagaaacatgacaagcgaggaaagaagagatgcccggatggaagctctgagagagagcatgccttgggagtccttcgccgacgacgtgagagctagaagagattttgtggtgccaacacggtacgtgcctagaaagggctctggagagctgttcgagcagaccgtctacagctacgtgggaagaaatgaaaagggccaggacctgggacggaagaaaggcgttgacaaggacaaggtgatgggcaacgccgtggtgagcgacgacggcaagtccgtgatcaaggtgagcgaaatgctgtgcctgagactgtggcacgaccctgagatcaagaagaaaggcaaagttgtcggcggctggtacgccgaccctgtgtacaagagcgacatccctgccctgaaggatggcacatacgtgcctagaatcgccaaggcccactgcggcagatccgcgtgggccccagtaccagagagactgctgctgcagcggcctgtggaactgtacctgggcgatgtggtgcgggtgaaggagatggtgggttgtctgagcggcttcgacatgggcggcgtggcctggaaattcgtggaccctatgaccagagagagcatcagcgcccctaccatcggctccctgaccggcaaggacgcccctaaggtggtgcggggcggcatcgtgggc

SEQ ID NO: 48 - DNA, 3168 bp, synthetic construct
  source 1..3168: mol_type = other DNA
atgatccccaatcccctgctgggcctggacaagcggactgccgacggcagtgagttcgagtctcccaagaaaaagaggaaagtgggatccgtggaagtgaaaaccgaagccgtgggcagactgggccggcctagatatctggtcctgggcatggaccccggcatcgcctcctgcggctttgccctcctggacatcaacaatcacgagatcctggaaatgggagttcggctgtttgacgcacctctgcaccctaaaactaagcagagcctggccgccatccggaggggatttcggtccacccggagaaacatcgaccggacccaggacaggctcaaacattgttttgagtgcttcaagaaatacggcgtggttcctgacgatgccgacaaggagtacctgcacaccgtgaaaggagataagcaacctctgaagctgagagtggacggtctgaacagaatgctgaccaatagagagtgggctatggccctgtattctctgtgcaagcggagagggtacatcccccacggcgagggagctcaggacaaaagcagcgacgatggaaaagtgctcgctgcaattagcgctaacgagaaggagattgccgccggaaagttcagaaccgccggcgaatggctggccagccggcccacctccagaaatagaggcggcaactacgaaatgtgcgtgaagcacgagcaattgatcgaggaaaccagaatcctgttcgaggcccagcggagcttcggcagtaaggtcgcttctgaggacttcgagcgggagtacatcgacatctgcaactgggaacggcctcgggacgacttcgaccggcgcacctacggcctggtggggagctgcgtgtacttcccccaggagaaaagagccgctcggtgcaccctgacatccgagctggtgtctgcctatggagcactgggcaatatcacaatcgtgctgggcgatggcggcgtgcgccggctgaccgccgccgaacgggacgagtgcatcagaatcctgttcaccgccgatcctaacgccaagaccagagtggtgaagttcggcgccttacgaaaaaagctggacctgtccagcagagactacttcaagggcgtgcccgccgatgatgaaaagaccagagaagtctttaagccaagcggctggcgcgccctgtacagtacactgagagagagcaacccccagctgctggcccgattacgagatgatatcgatctcgccgatgctgtgatggaagccgctgcttacgccagcagcgccagctctctggaaaactgcctgagccagctgcctctgagcgacgacgagaagcaggccttgtgcgccctgccatactcaagcaaagtgctgaacggctacggcagcagatctaagaaggccctggaccttctgctggcctgtctcaaggagcctgaagttctcaacctgaccgatgctgaagctgcttgtggcctcgatgccctgagactgggcggtcctcagctggaacggagtgaaaagctgataccctacgaggcctgggtgagcgctgccggccgtaccaacaacaatcccgtggtgattagagccatgagccaaatgagaaaggtggtcaacgccatctgcggcaagtggggcgtgccaaatgagatccacgtggaactggatcgggaactgcggctgcccaagcgggctaaagaagagatcgccaaggccaacaagaagaacgagaaagagagagagagaatcgccgtgcaagccgccgacatactgagttgttctcctgatgaggtgtctggcaagcagatcgagaagtacagactgtgggaggaacaggagaatagcgacctgtacacaggcgagagaatccacgtccagagacttctgagcgacgatacctatacacagatcgaccacatcctgcccttcagccgcacaggcgagaacagcagaaacaacaaggtgctcgtgctgtccgaaagcaaccagcagaagagagagcagaccccttatgagtggatgaacagcggacagcctggcgctcccagctgggacgagttcaagctgcgggtgcaggccaaccagcacctgaagcataggaagaaagagttcctgctggagcaggatctggaatctaaggaagtggagttcctgcagagaaacgtgaccgacaccgcctacatgagccgggaagtgtgcgcctacctgagcgactgcctgctcttccctgacgacggcatgaaggtgcacgtggtgcccacaacaggcagagccacagcctggctgagaagaagatggggactgaacttcggcgctaccggcgagaaagacagaagcgatgaccggcaccacgccaccgacgcttgcgtgatcgccgcctgtagcagaggcctggtgatcaagacagccaaggtcatgaagggcacccactggagcatcaccagaaacatgacaagcgaggaaagaagagatgcccggatggaagctctgagagagagcatgccttgggagtccttcgccgacgacgtgagagctagaagagattttgtggtgccaacacggtacgtgcctagaaagggctctggagagctgttcgagcagaccgtctacagctacgtgggaagaaatgaaaagggccaggacctgggacggaagaaaggcgttgacaaggacaaggtgatgggcaacgccgtggtgagcgacgacggcaagtccgtgatcaaggtgagcgaaatgctgtgcctgagactgtggcacgaccctgagatcaagaagaaaggcaaagttgtcggcggctggtacgccgaccctgtgtacaagagcgacatccctgccctgaaggatggcacatacgtgcctagaatcgccaaggcccactgcggcagatccgcgtgggccccagtaccagagagactgctgctgcagcggcctgtggaactgtacctgggcgatgtggtgcgggtgaaggagatggtgggttgtctgagcggcttcgacatgggcggcgtggcctggaaattcgtggaccctatgaccagagagagcatcagcgcccctaccatcggctccctgaccggcaaggacgcccctaaggtggtgcggggcggcatcgtgggctctagaaagcggacagcagacggctccgaatttgaaagccctaagaaaaagagaaaggtgtga

SEQ ID NO: 49 - protein, 1007 aa, synthetic construct
VEVKTEAVGRLGRPRYLVLGMDPGIASCGFALLDVNNHEILEMGVRLFDAPLHPKTKQSLAAIRRGFRSTRRNIDRTQDRLKHCLKCFKKYGVVPDDADKEYLHMVKGDKRPLELRVDGLNRMLTNREWGMVLYSLCKRRGYIPHGEGAQDKSSDDGKVLAAISANEKEIAAGEFKTVGEWLASRPASRNHGGNYKMCVKHEQLIEETRILFEAQRSFGSKVASEDFEREYIDICNWERPRDDFDRRTYGLVGSCVYFPQEKRAARCTLTSELVSAYGALGNITIVLGDGGTRRLTAAERNECICVLFTADPNAKTRVVKFGALRKKLDLGSRDYFKGVPADDEKTREVFKPSGWRALYSTLRESNPQLLARLKNDIDLADAVMEAAAYASSASSLDNCLSQLPLDDDEKQALCALPYSSKVLNGYGSRSKKALDLLLACLEEPEVLNLTDAEAACGLDAMRLGGPQLERSEKLIPYEAWVSATGRTNNNPVVIRAMSQMRKVVNAICGKWGVPNEIHVELDRELRLPKRAKEEIAKANRKNEKERERIAIQAADILSCSPDEVSGKQIEKYRLWEEQENSDLYTGERIHVQRLLNDDTYTQIDHILPFSRTGENSRNNKVLVLSESNQQKREQTPYEWMNSGHPGAPSWDEFKLRVQANQHLKHRKKEFLLEQDLESKEVEFLQRNVTDTAYMSREVCAYLSDCLLFPDDGMKVHVVPTTGRATAWLRRRWGLNFGATGEKDRSDDRHHATDACVIAACSRSLVIKTAKVMKGTHWSVTRHMTPEERRDARMEALRESMPWESFANDVRDRRDSVVPTRYVPRKGSGELFEQTVYSYAGEVDSLSRQVIGKSGGKASPKANVFILDDGKSALLVGDMLCLRLWFDPEVKKKGKVVGGWYANPLYKADLPDLRKGTYVPKAVKPRHGRSAWLEIPERMLEQKPVEIYRGDLVEIDGKNGCFVGYNIANGSWTFVDPLTREEIDMPSLASLSSQTNLKVVRRGLLPMD

SEQ ID NO: 50 - protein, 1008 aa, synthetic construct
MVEVKTEAVGRLGRPRYLVLGMDPGIASCGFALLDVNNHEILEMGVRLFDAPLHPKTKQSLAAIRRGFRSTRRNIDRTQDRLKHCLKCFKKYGVVPDDADKEYLHMVKGDKRPLELRVDGLNRMLTNREWGMVLYSLCKRRGYIPHGEGAQDKSSDDGKVLAAISANEKEIAAGEFKTVGEWLASRPASRNHGGNYKMCVKHEQLIEETRILFEAQRSFGSKVASEDFEREYIDICNWERPRDDFDRRTYGLVGSCVYFPQEKRAARCTLTSELVSAYGALGNITIVLGDGGTRRLTAAERNECICVLFTADPNAKTRVVKFGALRKKLDLGSRDYFKGVPADDEKTREVFKPSGWRALYSTLRESNPQLLARLKNDIDLADAVMEAAAYASSASSLDNCLSQLPLDDDEKQALCALPYSSKVLNGYGSRSKKALDLLLACLEEPEVLNLTDAEAACGLDAMRLGGPQLERSEKLIPYEAWVSATGRTNNNPVVIRAMSQMRKVVNAICGKWGVPNEIHVELDRELRLPKRAKEEIAKANRKNEKERERIAIQAADILSCSPDEVSGKQIEKYRLWEEQENSDLYTGERIHVQRLLNDDTYTQIDHILPFSRTGENSRNNKVLVLSESNQQKREQTPYEWMNSGHPGAPSWDEFKLRVQANQHLKHRKKEFLLEQDLESKEVEFLQRNVTDTAYMSREVCAYLSDCLLFPDDGMKVHVVPTTGRATAWLRRRWGLNFGATGEKDRSDDRHHATDACVIAACSRSLVIKTAKVMKGTHWSVTRHMTPEERRDARMEALRESMPWESFANDVRDRRDSVVPTRYVPRKGSGELFEQTVYSYAGEVDSLSRQVIGKSGGKASPKANVFILDDGKSALLVGDMLCLRLWFDPEVKKKGKVVGGWYANPLYKADLPDLRKGTYVPKAVKPRHGRSAWLEIPERMLEQKPVEIYRGDLVEIDGKNGCFVGYNIANGSWTFVDPLTREEIDMPSLASLSSQTNLKVVRRGLLPMD

SEQ ID NO: 51 - protein, 1064 aa, synthetic construct
KLEFATTMIPNPLLGLDKRTADGSEFESPKKKRKVGSVEVKTEAVGRLGRPRYLVLGMDPGIASCGFALLDVNNHEILEMGVRLFDAPLHPKTKQSLAAIRRGFRSTRRNIDRTQDRLKHCLKCFKKYGVVPDDADKEYLHMVKGDKRPLELRVDGLNRMLTNREWGMVLYSLCKRRGYIPHGEGAQDKSSDDGKVLAAISANEKEIAAGEFKTVGEWLASRPASRNHGGNYKMCVKHEQLIEETRILFEAQRSFGSKVASEDFEREYIDICNWERPRDDFDRRTYGLVGSCVYFPQEKRAARCTLTSELVSAYGALGNITIVLGDGGTRRLTAAERNECICVLFTADPNAKTRVVKFGALRKKLDLGSRDYFKGVPADDEKTREVFKPSGWRALYSTLRESNPQLLARLKNDIDLADAVMEAAAYASSASSLDNCLSQLPLDDDEKQALCALPYSSKVLNGYGSRSKKALDLLLACLEEPEVLNLTDAEAACGLDAMRLGGPQLERSEKLIPYEAWVSATGRTNNNPVVIRAMSQMRKVVNAICGKWGVPNEIHVELDRELRLPKRAKEEIAKANRKNEKERERIAIQAADILSCSPDEVSGKQIEKYRLWEEQENSDLYTGERIHVQRLLNDDTYTQIDHILPFSRTGENSRNNKVLVLSESNQQKREQTPYEWMNSGHPGAPSWDEFKLRVQANQHLKHRKKEFLLEQDLESKEVEFLQRNVTDTAYMSREVCAYLSDCLLFPDDGMKVHVVPTTGRATAWLRRRWGLNFGATGEKDRSDDRHHATDACVIAACSRSLVIKTAKVMKGTHWSVTRHMTPEERRDARMEALRESMPWESFANDVRDRRDSVVPTRYVPRKGSGELFEQTVYSYAGEVDSLSRQVIGKSGGKASPKANVFILDDGKSALLVGDMLCLRLWFDPEVKKKGKVVGGWYANPLYKADLPDLRKGTYVPKAVKPRHGRSAWLEIPERMLEQKPVEIYRGDLVEIDGKNGCFVGYNIANGSWTFVDPLTREEIDMPSLASLSSQTNLKVVRRGLLPMDSRKRTADGSEFESPKKKRKV

SEQ ID NO: 52 - DNA, 3027 bp, synthetic construct
  source 1..3027: mol_type = other DNA
atggtcgaggttaaaacagaggcggtcggtcggctcggtcgtcctcgatatctggttttgggcatggatccgggcattgcgagttgcggcttcgcgttgctcgacgtcaacaatcatgagatcttggaaatgggcgttcgtttgttcgatgctccgttgcatcctaaaacgaagcagagcttggccgccattcgtcgcggcttccgttcgacgcggcgtaatatcgaccgaacgcaagatcgactcaagcattgcctcaagtgctttaagaaatacggtgtcgttccggatgatgccgataaagagtaccttcatatggtcaagggggataaacggcccctcgagcttcgcgtcgacggcctgaaccgcatgctcacgaacagggagtggggcatggtcctttattccctttgcaagcgtcgcggctacattccgcacggcgaaggcgctcaggataaatcgagcgatgacggcaaagttcttgccgcgatcagcgcgaatgaaaaagaaatcgccgcaggtgaattcaaaaccgtaggcgaatggcttgcgtctcgtcccgctagcaggaaccacgggggcaattacaagatgtgcgtcaagcatgagcagcttatcgaagaaacccgtattctgttcgaagcacagcgctcgttcggttccaaggttgcttctgaagatttcgagcgcgaatacatcgacatatgcaattgggaacggccgcgcgacgatttcgataggcgcacgtacggcctggtggggtcatgcgtttactttccacaagagaagcgcgcggcacgctgcacgcttacaagcgagcttgtttctgcgtatggcgctttgggaaatataaccatcgtgctcggcgatggcggcacacgccgtcttacggcggccgaacgaaacgaatgcatctgcgtattgtttacggccgatccgaatgcgaagacccgcgttgtgaagttcggcgccttgcgcaaaaagctcgacttgggttcgcgtgattatttcaagggcgttcctgccgatgacgagaagacccgcgaggtgttcaagcccagcggttggcgcgccttgtattcgactttgagggaatctaatccgcagcttctcgcaaggctgaagaatgatatcgatttggcggatgccgttatggaggccgccgcgtatgcgtcgtctgcatcgagcttggacaattgcctcagccagcttcctcttgatgacgatgaaaaacaggcgctttgcgcgctgccgtattcgtctaaagtgctgaatggatacggaagtcgttcaaagaaagcgctcgacttgctgcttgcttgccttgaagagcctgaagttcttaatctgaccgatgcagaggcggcatgcgggttggatgcgatgcgtcttggcggtcctcagctcgaacgctctgagaaactaattccatatgaggcttgggtaagcgcgacgggtcgcacgaataataatcctgtcgtcattcgcgctatgtcgcaaatgcgcaaggtcgtaaatgccatatgcggcaaatggggcgttccgaacgaaatccacgttgagcttgaccgcgagcttcggttgcccaagcgcgcgaaagaagaaatcgctaaggccaataggaaaaacgagaaagagcgcgagcgcatagccattcaggctgcggacattctttcctgcagccccgatgaggtgagtgggaaacagatagagaagtatcgcttgtgggaggagcaggaaaactccgatttgtatacgggtgagcgcattcatgtccaacgccttttgaacgatgacacctatacgcagattgaccatattcttccgttttcgcgaacgggagagaattcgcgcaataacaaagtgctcgtgctttcggagagcaatcagcagaaacgcgagcagactccgtatgaatggatgaattcgggccatcctggcgctccgtcgtgggacgagttcaagcttcgagttcaggcgaatcagcaccttaaacatcgtaagaaagaattcttgctcgaacaggatttggaatcgaaggaagtcgaatttctccagcgaaacgtgaccgacacggcctatatgtcgcgcgaagtctgcgcctatctatccgattgtctgctgttccccgatgatggcatgaaggttcacgttgtccccacgacgggtcgtgctacggcatggttgcgtcgccgatgggggctgaacttcggagcaacgggggagaaggatcgttcggacgatcgtcatcatgccacggatgcttgcgttattgccgcatgcagcaggagcctcgtcatcaagacggcaaaggtcatgaagggaacgcattggagtgttacgcgtcatatgacgccggaggagcgcagagatgctcgtatggaggcgttgcgtgagagcatgccctgggaaagctttgccaacgatgtccgcgatcgtcgcgattccgtcgtgcccacgcgctatgtgcctcgcaaaggcagcggtgagcttttcgagcagacggtttattcgtatgcgggagaagtagattcgttgagtcggcaagtaattggcaagtcgggtggcaaagcatctccgaaggcgaatgttttcatccttgatgatggaaaatcagcactgttggttggcgacatgctttgcctgagactgtggtttgatccggaagtgaagaagaaaggcaaagttgttggaggttggtatgctaatccactgtacaaagccgatcttccagacctgcggaagggtacgtatgtcccgaaggcggttaagccaaggcatggaaggtctgcatggcttgagattccagaacgcatgcttgagcagaagccggtagagatataccgtggagacttggttgaaattgatggaaagaatggatgctttgttggatacaacatcgctaacggatcatggacctttgttgaccctctaacgcgcgaggagattgacatgccttccctggccagcctttcgtcgcaaacgaacttgaaggtcgtgcgaagagggctgcttcccatggattga

SEQ ID NO: 53 - DNA, 3021 bp, synthetic construct
  source 1..3021: mol_type = other DNA
gtcgaggtgaaaaccgaggccgtgggcagactgggcagacctagatacctggtgctgggcatggaccccggcatcgcttcctgcggattcgccctgctggatgttaacaatcacgagatcctggagatgggcgtgcggctgtttgatgctcctctgcaccccaaaactaagcagagcctggccgccatcaggcgcggatttagatccactagaagaaacattgacagaacccaggacagactgaaacactgcctgaagtgcttcaagaaatacggcgtggttccagatgacgctgacaaggaatacctgcacatggtgaagggggataagcggcctctggaactgagagtggacggcctgaaccggatgctgacaaacagagagtggggaatggttctgtactctctgtgcaagagaagaggctacatcccacacggagagggcgcccaggacaagagctccgacgacggcaaggtgctggccgctatcagcgccaacgagaaagaaatcgcagccggagagttcaagacagtgggcgagtggctggccagcagacctgccagcagaaaccacggcggcaattacaaaatgtgcgtgaagcacgagcaactcatcgaggagacaagaatcctgttcgaggcccagcggtcttttggcagcaaggtcgcctccgaggatttcgaaagagagtacatcgatatctgcaactgggaaagacctagagatgacttcgatagacggacatacggcctggtgggcagctgtgtgtacttccctcaggagaagcgggccgccagatgcaccctgaccagcgagctggtcagcgcttatggcgccctgggcaacatcaccatcgtgctgggcgacggcggcaccagacggcttacagccgctgagagaaatgagtgcatctgtgtgctgttcacagccgatcccaacgccaagacccgggtggtgaagttcggcgctcttagaaagaagctggacctgggcagtagagactacttcaagggcgtgcccgctgatgacgaaaagaccagagaagtgttcaagccttctggctggcgggctctgtacagcaccctgcgggagagcaacccccagctgctggccagactgaagaatgacatcgatctcgccgacgccgtgatggaagccgccgcctacgccagcagcgcctctagcctggacaactgcctgagtcagcttcccctggacgatgacgagaagcaagccctgtgcgccctgccttacagcagcaaggtgctgaacggctacggcagcagaagcaagaaagccctggacctgctgctggcctgcctggaggagcctgaggtgctgaacctgacagatgccgaggccgcttgtggactggatgctatgagactgggaggtcctcagctggaacggagcgagaagttgatcccctatgaggcctgggtgagcgccacaggtagaaccaacaacaaccccgtggtgatccgggctatgagccagatgcggaaagtggtgaacgccatctgcggcaagtggggcgtgcctaacgagatccacgtggaactggatagagagctgagactgcctaagcgcgccaaagaggaaatcgccaaggccaaccggaagaacgagaaagaaagagaacggatcgccatccaggccgccgacatcctgtcttgcagccctgacgaggtgagcggcaaacagatcgagaagtacagactgtgggaggaacaggagaacagcgacctgtacaccggcgaaagaatccacgtgcagagactactaaatgacgacacctatacacagattgaccacatcctgcccttcagcagaacaggcgaaaacagcagaaacaacaaggtgcttgtgctcagcgaatctaaccagcagaagagagagcagaccccatacgagtggatgaacagcggccaccctggcgccccttcttgggacgagttcaagctgcgcgttcaggccaatcaacacctgaagcacagaaagaaggagttcctgctggaacaagatctggaatccaaggaggtggagttcctgcagagaaacgtgaccgataccgcctacatgagccgggaggtttgtgcctacctgagcgactgcctgctgtttccggacgacggcatgaaagtgcatgtggtgcctaccaccggcagagccaccgcatggctgagaagaagatggggcctgaacttcggcgccaccggagagaaggacaggagcgacgatagacaccacgccacagacgcttgtgtgatcgccgcctgttcccggagcctcgtgatcaagaccgctaaggtcatgaaaggaacccactggtccgtgacacgccacatgacacctgaggaacggagggatgccagaatggaagccctgagggaaagcatgccctgggagtctttcgctaacgacgtgcgggacagaagagacagcgtggtgcctacccggtacgtgcctagaaagggctctggcgagctgttcgagcagacagtgtacagctacgccggcgaagtggattctctgtccagacaggtgatcggaaagagcggcggcaaagcctctcctaaggccaacgtgttcatcctggacgacggcaagagcgccctgctggtcggcgacatgctgtgcctgagactgtggttcgacccagaagtcaagaaaaagggcaaggtggtgggtggatggtacgcgaatcccctgtataaggccgacctgcctgatctgcggaagggcacatacgtgcctaaggccgtgaaaccaagacacggcagaagcgcctggctggagatccccgagagaatgctggaacagaaacctgtggagatctaccggggcgacctggttgaaatcgatggcaagaacggttgttttgtgggatacaacatcgctaatggctcttggaccttcgtggaccctcttacccgggaagagatcgacatgcctagcctggcaagcctgtccagccagaccaacctgaaggtggttagacgcggcctgctgcctatggac

SEQ ID NO: 54 - DNA, 3192 bp, synthetic construct
  source 1..3192: mol_type = other DNA
aagcttgaattcgccaccatgatccccaatcccctgctgggcctggacaagcggactgccgacggcagtgagttcgagtctcccaagaaaaagaggaaagtgggatccgtcgaggtgaaaaccgaggccgtgggcagactgggcagacctagatacctggtgctgggcatggaccccggcatcgcttcctgcggattcgccctgctggatgttaacaatcacgagatcctggagatgggcgtgcggctgtttgatgctcctctgcaccccaaaactaagcagagcctggccgccatcaggcgcggatttagatccactagaagaaacattgacagaacccaggacagactgaaacactgcctgaagtgcttcaagaaatacggcgtggttccagatgacgctgacaaggaatacctgcacatggtgaagggggataagcggcctctggaactgagagtggacggcctgaaccggatgctgacaaacagagagtggggaatggttctgtactctctgtgcaagagaagaggctacatcccacacggagagggcgcccaggacaagagctccgacgacggcaaggtgctggccgctatcagcgccaacgagaaagaaatcgcagccggagagttcaagacagtgggcgagtggctggccagcagacctgccagcagaaaccacggcggcaattacaaaatgtgcgtgaagcacgagcaactcatcgaggagacaagaatcctgttcgaggcccagcggtcttttggcagcaaggtcgcctccgaggatttcgaaagagagtacatcgatatctgcaactgggaaagacctagagatgacttcgatagacggacatacggcctggtgggcagctgtgtgtacttccctcaggagaagcgggccgccagatgcaccctgaccagcgagctggtcagcgcttatggcgccctgggcaacatcaccatcgtgctgggcgacggcggcaccagacggcttacagccgctgagagaaatgagtgcatctgtgtgctgttcacagccgatcccaacgccaagacccgggtggtgaagttcggcgctcttagaaagaagctggacctgggcagtagagactacttcaagggcgtgcccgctgatgacgaaaagaccagagaagtgttcaagccttctggctggcgggctctgtacagcaccctgcgggagagcaacccccagctgctggccagactgaagaatgacatcgatctcgccgacgccgtgatggaagccgccgcctacgccagcagcgcctctagcctggacaactgcctgagtcagcttcccctggacgatgacgagaagcaagccctgtgcgccctgccttacagcagcaaggtgctgaacggctacggcagcagaagcaagaaagccctggacctgctgctggcctgcctggaggagcctgaggtgctgaacctgacagatgccgaggccgcttgtggactggatgctatgagactgggaggtcctcagctggaacggagcgagaagttgatcccctatgaggcctgggtgagcgccacaggtagaaccaacaacaaccccgtggtgatccgggctatgagccagatgcggaaagtggtgaacgccatctgcggcaagtggggcgtgcctaacgagatccacgtggaactggatagagagctgagactgcctaagcgcgccaaagaggaaatcgccaaggccaaccggaagaacgagaaagaaagagaacggatcgccatccaggccgccgacatcctgtcttgcagccctgacgaggtgagcggcaaacagatcgagaagtacagactgtgggaggaacaggagaacagcgacctgtacaccggcgaaagaatccacgtgcagagactactaaatgacgacacctatacacagattgaccacatcctgcccttcagcagaacaggcgaaaacagcagaaacaacaaggtgcttgtgctcagcgaatctaaccagcagaagagagagcagaccccatacgagtggatgaacagcggccaccctggcgccccttcttgggacgagttcaagctgcgcgttcaggccaatcaacacctgaagcacagaaagaaggagttcctgctggaacaagatctggaatccaaggaggtggagttcctgcagagaaacgtgaccgataccgcctacatgagccgggaggtttgtgcctacctgagcgactgcctgctgtttccggacgacggcatgaaagtgcatgtggtgcctaccaccggcagagccaccgcatggctgagaagaagatggggcctgaacttcggcgccaccggagagaaggacaggagcgacgatagacaccacgccacagacgcttgtgtgatcgccgcctgttcccggagcctcgtgatcaagaccgctaaggtcatgaaaggaacccactggtccgtgacacgccacatgacacctgaggaacggagggatgccagaatggaagccctgagggaaagcatgccctgggagtctttcgctaacgacgtgcgggacagaagagacagcgtggtgcctacccggtacgtgcctagaaagggctctggcgagctgttcgagcagacagtgtacagctacgccggcgaagtggattctctgtccagacaggtgatcggaaagagcggcggcaaagcctctcctaaggccaacgtgttcatcctggacgacggcaagagcgccctgctggtcggcgacatgctgtgcctgagactgtggttcgacccagaagtcaagaaaaagggcaaggtggtgggtggatggtacgcgaatcccctgtataaggccgacctgcctgatctgcggaagggcacatacgtgcctaaggccgtgaaaccaagacacggcagaagcgcctggctggagatccccgagagaatgctggaacagaaacctgtggagatctaccggggcgacctggttgaaatcgatggcaagaacggttgttttgtgggatacaacatcgctaatggctcttggaccttcgtggaccctcttacccgggaagagatcgacatgcctagcctggcaagcctgtccagccagaccaacctgaaggtggttagacgcggcctgctgcctatggactctagaaagcggacagcagacggctccgaatttgaaagccctaagaaaaagagaaaggtgtga

SEQ ID NO: 55 - protein, 1018 aa, synthetic construct
PMAVNREVGKLGNPRYLVLGLDPGIASCGFALLDLNNHEILEMGSRLFDAPTVPKTGQSKAVVRRMHRSTRRNLDRTQDRLKHCLNALKEADLVPEDATKEYFHTQKGDKPPLRLRVDGLDRRLTEREWAIVLYSLCKRRGYIPHGEGSVDGSSEDGKVLAAVSANAKLLDEGKYRTVGEWLASQERSRNRAGSYEKCVTHKLLMEEASTLFAKQRGFGNEYASPELEKKYVGICDWEKPRDAFDERVYQTQVGHCIYFHDEKRAARSTLSNELVSAYAAFGNISIVQPGHETRRVSPDVRDQCIGLLFSASPDGPASGTKVTYKSLRKLLDLSAKASFKGINVDDEKSREVFQPKGWRALRATLVDSAPELLMRLRTDIDLADEVFEAAAYSSSIEVLRNRLGLLDLSDEEVDKLSRLPYASRVLNGYGSRSKKALDILLDQFEDPEILDLAEAERSSGLLGKRMAGNGIAKSDRLMPYKCWLEDTGRTNNNPVVLRAIAQMRKVVNAICREWGVPHEIHVELDRELKLSKKQQDKIRKNNKKNEAENARVAKQIAGLLGCDPGSVSRNQIRKYTLWEQQDNVDIYTGDAIDLERLLADSMYTQIDHVLPFSRTGDNAWYNKVLCLASSNQHKRERSPYEWMTSGEAGAPSWEEFQARVVENHKLGYKKRSFLLEENLDAKVDDFLRRSITDTAYMSRELSAYLDDCLEFPEGRTKVPVQPTNGRATAWLRHAWGLNFGAQGEKDREDDRHHATDACVIAACNRSMLIKTAQYSSGKRKMSEEQKDAELREAQPWPTFSKDVTTRRDCVIPTRFVPHKAGGEIFEQTLYAYKGTNAQGKHILAKTDASTGKEKETVAGNAVVSVDGKSAVIVSEMLCLMLWHDADARPNGKVKGQWYADPIYRADLPALRDGTFVHRIAKAHKGRDVWPEIPDRVLENTKPIVVYRGDVLMVDDAIGRFAGIDIDGVRWEFAQISPDVPAEIDTIGSLGNENKVKLVTEDVLGHCWGQLNMKVGCAE

SEQ ID NO: 56 - protein, 1019 aa, synthetic construct
MPMAVNREVGKLGNPRYLVLGLDPGIASCGFALLDLNNHEILEMGSRLFDAPTVPKTGQSKAVVRRMHRSTRRNLDRTQDRLKHCLNALKEADLVPEDATKEYFHTQKGDKPPLRLRVDGLDRRLTEREWAIVLYSLCKRRGYIPHGEGSVDGSSEDGKVLAAVSANAKLLDEGKYRTVGEWLASQERSRNRAGSYEKCVTHKLLMEEASTLFAKQRGFGNEYASPELEKKYVGICDWEKPRDAFDERVYQTQVGHCIYFHDEKRAARSTLSNELVSAYAAFGNISIVQPGHETRRVSPDVRDQCIGLLFSASPDGPASGTKVTYKSLRKLLDLSAKASFKGINVDDEKSREVFQPKGWRALRATLVDSAPELLMRLRTDIDLADEVFEAAAYSSSIEVLRNRLGLLDLSDEEVDKLSRLPYASRVLNGYGSRSKKALDILLDQFEDPEILDLAEAERSSGLLGKRMAGNGIAKSDRLMPYKCWLEDTGRTNNNPVVLRAIAQMRKVVNAICREWGVPHEIHVELDRELKLSKKQQDKIRKNNKKNEAENARVAKQIAGLLGCDPGSVSRNQIRKYTLWEQQDNVDIYTGDAIDLERLLADSMYTQIDHVLPFSRTGDNAWYNKVLCLASSNQHKRERSPYEWMTSGEAGAPSWEEFQARVVENHKLGYKKRSFLLEENLDAKVDDFLRRSITDTAYMSRELSAYLDDCLEFPEGRTKVPVQPTNGRATAWLRHAWGLNFGAQGEKDREDDRHHATDACVIAACNRSMLIKTAQYSSGKRKMSEEQKDAELREAQPWPTFSKDVTTRRDCVIPTRFVPHKAGGEIFEQTLYAYKGTNAQGKHILAKTDASTGKEKETVAGNAVVSVDGKSAVIVSEMLCLMLWHDADARPNGKVKGQWYADPIYRADLPALRDGTFVHRIAKAHKGRDVWPEIPDRVLENTKPIVVYRGDVLMVDDAIGRFAGIDIDGVRWEFAQISPDVPAEIDTIGSLGNENKVKLVTEDVLGHCWGQLNMKVGCAE

SEQ ID NO: 57 - protein, 1068 aa, synthetic construct
MIPNPLLGLDKRTADGSEFESPKKKRKVGSPMAVNREVGKLGNPRYLVLGLDPGIASCGFALLDLNNHEILEMGSRLFDAPTVPKTGQSKAVVRRMHRSTRRNLDRTQDRLKHCLNALKEADLVPEDATKEYFHTQKGDKPPLRLRVDGLDRRLTEREWAIVLYSLCKRRGYIPHGEGSVDGSSEDGKVLAAVSANAKLLDEGKYRTVGEWLASQERSRNRAGSYEKCVTHKLLMEEASTLFAKQRGFGNEYASPELEKKYVGICDWEKPRDAFDERVYQTQVGHCIYFHDEKRAARSTLSNELVSAYAAFGNISIVQPGHETRRVSPDVRDQCIGLLFSASPDGPASGTKVTYKSLRKLLDLSAKASFKGINVDDEKSREVFQPKGWRALRATLVDSAPELLMRLRTDIDLADEVFEAAAYSSSIEVLRNRLGLLDLSDEEVDKLSRLPYASRVLNGYGSRSKKALDILLDQFEDPEILDLAEAERSSGLLGKRMAGNGIAKSDRLMPYKCWLEDTGRTNNNPVVLRAIAQMRKVVNAICREWGVPHEIHVELDRELKLSKKQQDKIRKNNKKNEAENARVAKQIAGLLGCDPGSVSRNQIRKYTLWEQQDNVDIYTGDAIDLERLLADSMYTQIDHVLPFSRTGDNAWYNKVLCLASSNQHKRERSPYEWMTSGEAGAPSWEEFQARVVENHKLGYKKRSFLLEENLDAKVDDFLRRSITDTAYMSRELSAYLDDCLEFPEGRTKVPVQPTNGRATAWLRHAWGLNFGAQGEKDREDDRHHATDACVIAACNRSMLIKTAQYSSGKRKMSEEQKDAELREAQPWPTFSKDVTTRRDCVIPTRFVPHKAGGEIFEQTLYAYKGTNAQGKHILAKTDASTGKEKETVAGNAVVSVDGKSAVIVSEMLCLMLWHDADARPNGKVKGQWYADPIYRADLPALRDGTFVHRIAKAHKGRDVWPEIPDRVLENTKPIVVYRGDVLMVDDAIGRFAGIDIDGVRWEFAQISPDVPAEIDTIGSLGNENKVKLVTEDVLGHCWGQLNMKVGCAESRKRTADGSEFESPKKKRKV

SEQ ID NO: 58 - DNA, 3060 bp, synthetic construct
  source 1..3060: mol_type = other DNA
atgcccatggccgtcaaccgtgaagtcggcaagttggggaatcctcgctatttggtgcttggtctcgacccggggattgccagctgcgggtttgctctccttgacctcaacaatcacgagatcctcgagatggggtcgcgcctatttgacgccccgacagttcctaagaccggccagagcaaggcggtcgtgaggcgtatgcatcgctcgacccgccgcaacctcgacaggacccaagaccgtctcaagcattgtctcaatgccctaaaggaagccgacttggtcccggaggatgccaccaaggagtacttccatacgcagaagggtgacaagccgccgctgcggctcagagtcgacgggctcgataggaggctgacagagcgcgaatgggccatcgtgctctattccctgtgcaagcgtcgtggctacattccccatggtgaggggagcgttgatggctccagcgaggacggcaaggtgctggctgccgtctctgccaatgccaagctgttggacgagggaaagtatcgcacggtaggtgagtggctggcttcgcaggagcgcagtcgcaatagggccggatcgtatgagaagtgcgtcacccacaagctgctgatggaagaggcctcgacgctctttgccaagcagaggggcttcggcaacgaatacgcatcgccggaactcgagaagaaatatgtcgggatctgcgattgggaaaagccgcgagatgccttcgatgagcgcgtctatcagacgcaagttggccattgcatctattttcatgatgagaaacgcgccgctcggtccacgctttccaacgagctcgtttccgcctatgcggcctttggcaacatctcgatagtccagcctggtcatgagacgcgtcgggtcagtcctgatgttcgcgaccagtgcatcggcttgctgttctcggcgtcacccgatggcccagcctcaggcactaaggtcacctataagagcctgaggaagctcctggacctctccgccaaggcctcctttaaaggaatcaatgtcgatgacgagaaatcgcgcgaggtctttcagccaaagggctggcgagccctgagggctacgttggtcgatagcgcccccgagctcctcatgaggctcaggacggacatcgatcttgccgatgaggtcttcgaggcggcggcttactcgtcatccatcgaggttctcaggaaccggcttggcctgctcgacctttctgacgaggaagtcgacaagctgagccgactgccgtatgcgagcagggtcctcaacggctatggctctaggtcgaagaaggcattggacatcctcttggatcagtttgaagatccagagatccttgacctcgccgaggcagagaggagctcggggcttcttgggaagcggatggccgggaacggcatcgccaagtccgatcgcctgatgccttataagtgctggctcgaagacactggacgtacgaacaacaatcctgtcgttctgcgcgcaatcgcccagatgcgcaaggtcgtcaacgccatatgccgagagtggggggtcccgcatgagatacatgtggagctcgatagggaattaaagctctcgaagaagcaacaagacaagatacggaaaaacaacaagaagaacgaggcggagaacgcgcgcgttgccaagcagattgccggtctgcttgggtgcgacccgggctccgtgagcaggaaccagattcgcaaatacaccctgtgggagcagcaggacaacgtcgacatctatacgggggacgcaatcgatctcgagcgcctgctcgcagacagcatgtacacccaaatagaccacgtcttgccgttctcgaggactggcgacaacgcttggtacaacaaggttctctgccttgcatcgagcaaccagcacaagcgagagcgctcaccttatgagtggatgacgtctggcgaagccggggctccctcttgggaggagtttcaggctcgtgtggtcgagaatcacaagcttggctataagaagcgctcgttccttctcgaagagaacctcgacgccaaggtcgatgactttcttagaaggagcatcactgacaccgcatacatgtctcgcgaactgagcgcgtatctcgacgactgcctcgagttccctgaggggagaacgaaggtccctgtccagccaacgaacggcagggcaaccgcgtggttgcggcatgcttgggggctgaacttcggcgcgcaaggcgagaaggaccgggaggacgaccgccatcacgccaccgatgcttgcgtgatcgctgcctgcaaccgctcaatgctcatcaagacggcgcagtattccagcggaaagcgcaagatgagcgaggagcagaaggatgccgagctgcgggaagcccagccctggccaacgttctccaaggatgtgacaacaaggcgggattgcgttattcccaccaggttcgtacctcataaggcaggaggagagatcttcgagcaaacgctttatgcctataagggaacgaacgcccaaggcaagcacatccttgccaagaccgatgcgtcaaccggcaaggaaaaggaaacggttgccgggaatgccgttgtttctgtagatgggaagtcggcggtcatcgtcagcgagatgctttgcttgatgctgtggcatgacgcagacgcacggcccaacggcaaggtcaaaggccagtggtatgccgaccccatctatcgcgccgatttgcctgctctgcgagacggcaccttcgtccatcgcattgcaaaggcgcataaaggaagggacgtttggcctgagatacccgaccgtgtgttggagaacaccaaaccgatagtcgtctatcgaggcgacgtcctcatggtggatgatgcgataggacgatttgcaggcattgatatcgatggtgttaggtgggaattcgctcaaatatctcctgatgttcccgcagagattgacaccataggaagcctcggcaacgagaacaaggtgaagctggttacagaggatgtccttggccactgttggggccaattaaatatgaaagtgggctgcgcagaataa

SEQ ID NO: 59 - DNA, 3054 bp, synthetic construct
  source 1..3054: mol_type = other DNA
cctatggccgtgaatcgggaagtgggaaagctgggcaatcctagatacctggtgctgggcctggaccctggcatcgccagctgcggcttcgccctgctggatctgaacaatcacgaaatccttgagatgggcagcagactgttcgacgcaccaacagtgcctaagaccggccagagcaaggccgtggtgcggagaatgcaccgctctacaagaagaaacctggataggacacaggaccggctgaagcactgtctgaatgccctcaaggaagccgatctggttccagaggacgcgacaaaggaatacttccacacccaaaagggcgataagcctccactgaggctgagagtggacggcctggaccggcggctgaccgagagagaatgggccatcgtgctgtacagcctgtgcaagagaagaggctacatcccccacggcgaaggctctgtggatggcagcagcgaggacggcaaggtcttggccgccgtgagcgccaacgctaagctgctggacgagggaaaatacagaaccgtgggagaatggctggccagtcaggagagatcccggaacagagccggctcctacgagaaatgcgtgacacacaaactgctgatggaagaggcttctactctttttgccaagcaaagaggcttcggcaacgagtatgccagccctgaattggaaaagaagtacgtgggtatttgtgattgggagaagcccagagatgcatttgacgaacgcgtctaccagacacaagtgggacattgcatctactttcacgatgaaaagagagctgccagaagcacgctgagcaacgagctggtgtcggcttacgcagctttcggcaacatctctatcgtgcagcctggccacgaaaccagacgggtcagccccgacgttcgggaccagtgcatcggcctgctgttcagcgcctccccagacggtcctgccagcggcacaaaggtgacctacaaaagcctgagaaagctgctggacctgtctgctaaggcctcttttaagggcatcaacgtggacgacgagaagagccgggaagttttccaacccaagggctggagagctctgagggccaccctggtggacagcgcccctgagctgctgatgagactcagaacagacatcgacctggccgacgaggtgttcgaggctgccgcttacagctcttctatcgaggtgctgagaaataggctgggcttgctggatctgagcgacgaggaagtggacaagctgtccagactgccatacgccagcagagtgctgaacggctacggcagccggtccaagaaggccctggacatcctgctggaccagttcgaggaccctgaaatcctggacctggctgaagccgaacggagcagcggcctgctcggcaagcggatggcgggcaacggcatcgctaaaagcgacagactgatgccttacaagtgctggctggaggacaccggcagaacaaacaacaaccctgtggtcctgcgggccatcgcccagatgcgcaaggtggtgaacgccatttgcagagagtggggcgtgcctcatgagatccacgtggaactggatagagaactgaagctgagcaaaaagcagcaggacaagatccggaagaacaacaagaagaacgaggccgaaaacgcccgggtggccaagcagatcgccggcctgctgggctgtgaccccggcagcgttagcagaaatcagatcagaaagtacaccctgtgggagcagcaagataatgtggacatctacaccggagatgccatcgatctggaacggctcctggctgacagtatgtacacccagatcgaccacgtgctccccttcagcagaaccggcgacaacgcctggtacaacaaagtgctctgcctggccagctccaaccagcacaagcgggagcggtcaccctacgagtggatgaccagcggcgaggccggagcccctagctgggaagagttccaggcccgagtggtggaaaaccacaagctgggatacaagaaaagatccttcctgctggaagagaatctggatgccaaggtggacgacttcctgagaagaagcatcaccgacaccgcctacatgagcagagagctgagcgcctatctggacgactgcctggaatttcctgagggccgtacaaaggtgcccgtgcagcctactaacggacgggctaccgcctggctccggcacgcctggggcctgaacttcggcgcccagggcgagaaggacagggaggacgacagacaccacgcgaccgacgcttgtgtgatcgctgcatgcaacagatctatgctgatcaagaccgcccaatacagcagcggaaaaagaaagatgagcgaggaacagaaagatgctgaactgcgggaggcccagccttggcccaccttttctaaggacgtgaccacccgccgggattgcgtgatccctacacggttcgtgcctcataaggccggcggcgagatcttcgagcagaccctgtacgcctacaagggcaccaacgcccagggaaagcacatcctggccaaaaccgacgcctctaccggcaaagagaaagagacagtggccggaaatgccgttgtttctgtggacggcaagtccgccgtgatcgtgtccgagatgctgtgtctgatgctgtggcacgacgccgacgccaggccgaacggtaaggtgaagggacagtggtatgccgaccccatctatcgggccgacctgcctgccctgagagatgggaccttcgtgcacagaatcgccaaagcccacaagggccgagatgtgtggcctgagattccagacagagtgctggagaacaccaagcctatcgtcgtgtaccggggcgacgtgctgatggtggatgatgccatcggcagattcgccggcatcgatatcgatggcgtgagatgggagttcgcccagattagccctgatgtgcctgccgaaatcgatacaatcggtagcctgggcaacgagaacaaggtgaagctcgtgacagaggatgtgctgggacactgctggggacagctgaacatgaaagtaggctgcgccgag

SEQ ID NO: 60 - DNA, 3207 bp, synthetic construct
  source 1..3207: mol_type = other DNA
atgatccccaatcccctgctgggcctggacaagcggactgccgacggcagtgagttcgagtctcccaagaaaaagaggaaagtgggatcccctatggccgtgaatcgggaagtgggaaagctgggcaatcctagatacctggtgctgggcctggaccctggcatcgccagctgcggcttcgccctgctggatctgaacaatcacgaaatccttgagatgggcagcagactgttcgacgcaccaacagtgcctaagaccggccagagcaaggccgtggtgcggagaatgcaccgctctacaagaagaaacctggataggacacaggaccggctgaagcactgtctgaatgccctcaaggaagccgatctggttccagaggacgcgacaaaggaatacttccacacccaaaagggcgataagcctccactgaggctgagagtggacggcctggaccggcggctgaccgagagagaatgggccatcgtgctgtacagcctgtgcaagagaagaggctacatcccccacggcgaaggctctgtggatggcagcagcgaggacggcaaggtcttggccgccgtgagcgccaacgctaagctgctggacgagggaaaatacagaaccgtgggagaatggctggccagtcaggagagatcccggaacagagccggctcctacgagaaatgcgtgacacacaaactgctgatggaagaggcttctactctttttgccaagcaaagaggcttcggcaacgagtatgccagccctgaattggaaaagaagtacgtgggtatttgtgattgggagaagcccagagatgcatttgacgaacgcgtctaccagacacaagtgggacattgcatctactttcacgatgaaaagagagctgccagaagcacgctgagcaacgagctggtgtcggcttacgcagctttcggcaacatctctatcgtgcagcctggccacgaaaccagacgggtcagccccgacgttcgggaccagtgcatcggcctgctgttcagcgcctccccagacggtcctgccagcggcacaaaggtgacctacaaaagcctgagaaagctgctggacctgtctgctaaggcctcttttaagggcatcaacgtggacgacgagaagagccgggaagttttccaacccaagggctggagagctctgagggccaccctggtggacagcgcccctgagctgctgatgagactcagaacagacatcgacctggccgacgaggtgttcgaggctgccgcttacagctcttctatcgaggtgctgagaaataggctgggcttgctggatctgagcgacgaggaagtggacaagctgtccagactgccatacgccagcagagtgctgaacggctacggcagccggtccaagaaggccctggacatcctgctggaccagttcgaggaccctgaaatcctggacctggctgaagccgaacggagcagcggcctgctcggcaagcggatggcgggcaacggcatcgctaaaagcgacagactgatgccttacaagtgctggctggaggacaccggcagaacaaacaacaaccctgtggtcctgcgggccatcgcccagatgcgcaaggtggtgaacgccatttgcagagagtggggcgtgcctcatgagatccacgtggaactggatagagaactgaagctgagcaaaaagcagcaggacaagatccggaagaacaacaagaagaacgaggccgaaaacgcccgggtggccaagcagatcgccggcctgctgggctgtgaccccggcagcgttagcagaaatcagatcagaaagtacaccctgtgggagcagcaagataatgtggacatctacaccggagatgccatcgatctggaacggctcctggctgacagtatgtacacccagatcgaccacgtgctccccttcagcagaaccggcgacaacgcctggtacaacaaagtgctctgcctggccagctccaaccagcacaagcgggagcggtcaccctacgagtggatgaccagcggcgaggccggagcccctagctgggaagagttccaggcccgagtggtggaaaaccacaagctgggatacaagaaaagatccttcctgctggaagagaatctggatgccaaggtggacgacttcctgagaagaagcatcaccgacaccgcctacatgagcagagagctgagcgcctatctggacgactgcctggaatttcctgagggccgtacaaaggtgcccgtgcagcctactaacggacgggctaccgcctggctccggcacgcctggggcctgaacttcggcgcccagggcgagaaggacagggaggacgacagacaccacgcgaccgacgcttgtgtgatcgctgcatgcaacagatctatgctgatcaagaccgcccaatacagcagcggaaaaagaaagatgagcgaggaacagaaagatgctgaactgcgggaggcccagccttggcccaccttttctaaggacgtgaccacccgccgggattgcgtgatccctacacggttcgtgcctcataaggccggcggcgagatcttcgagcagaccctgtacgcctacaagggcaccaacgcccagggaaagcacatcctggccaaaaccgacgcctctaccggcaaagagaaagagacagtggccggaaatgccgttgtttctgtggacggcaagtccgccgtgatcgtgtccgagatgctgtgtctgatgctgtggcacgacgccgacgccaggccgaacggtaaggtgaagggacagtggtatgccgaccccatctatcgggccgacctgcctgccctgagagatgggaccttcgtgcacagaatcgccaaagcccacaagggccgagatgtgtggcctgagattccagacagagtgctggagaacaccaagcctatcgtcgtgtaccggggcgacgtgctgatggtggatgatgccatcggcagattcgccggcatcgatatcgatggcgtgagatgggagttcgcccagattagccctgatgtgcctgccgaaatcgatacaatcggtagcctgggcaacgagaacaaggtgaagctcgtgacagaggatgtgctgggacactgctggggacagctgaacatgaaagtaggctgcgccgagtctagaaagcggacagcagacggctccgaatttgaaagccctaagaaaaagagaaaggtgtga

SEQ ID NO: 61 - RNA, 36 nt, synthetic construct
  source 1..36: mol_type = other RNA
gttctgctaccatcgaaatttttgctaggctacaac

SEQ ID NO: 62 - RNA, 78 nt, synthetic construct
  source 1..78: mol_type = other RNA
ttgtagtctagcaaaggttttgatgatctagcagaacaagggtttatcccggaatcgactccttagggagtctttttt

SEQ ID NO: 63 - RNA, 35 nt, synthetic construct
  source 1..35: mol_type = other RNA
gttttggtaccctctaaatttttgctatactgaaa

SEQ ID NO: 64 - RNA, 80 nt, synthetic construct
  source 1..80: mol_type = other RNA
cagtatagcaaaggtttagaggacctatcaaaacaagggaattattcccgaaatcggaactgctaagcagttcctttttt

SEQ ID NO: 65 - RNA, 36 nt, synthetic construct
  source 1..36: mol_type = other RNA
gttttgctaccctcacaatttttgctatgtaaagac

SEQ ID NO: 66 - RNA, 88 nt, synthetic construct
  source 1..88: mol_type = other RNA
ttacatagcaaagattgtgaggatctagcgaaacaagggctgtcttaggacatgtcccgaaatcgagtctcgtaggagactctttttt

SEQ ID NO: 67 - RNA, 36 nt, synthetic construct
  source 1..36: mol_type = other RNA
gttttagtacctgaaagaattgagttattgtaaaac

SEQ ID NO: 68 - RNA, 83 nt, synthetic construct
  source 1..83: mol_type = other RNA
gttttgcaataactcaattttttcagatctactaaaacaaggctttatgccgaaatcaaggacacagataagtgtcctttttt

SEQ ID NO: 69 - RNA, 36 nt, synthetic construct; identical to SEQ ID NO: 65
  source 1..36: mol_type = other RNA

SEQ ID NO: 70 - RNA, 88 nt, synthetic construct
  source 1..88: mol_type = other RNA
ttacatagcaaagattgtgaggatctagcaaaacaagggctgtcttaggacatgtcccgaaatcgagtctcgtaggagactctttttt

SEQ ID NO: 71 - RNA, 36 nt, synthetic construct
  source 1..36: mol_type = other RNA
gtcttgagctcgcacttttccccaagctgatacaat

SEQ ID NO: 72 - RNA, 111 nt, synthetic construct
  source 1..111: mol_type = other RNA
tcaccttggggaaaagtgcgagactccagacaaggggaacctacaacggtaggttcacccgtagggttacccccgcgtcatcttcggaaggcgcggggcgaactctttttt

SEQ ID NO: 73 - RNA, 36 nt, synthetic construct; identical to SEQ ID NO: 71
  source 1..36: mol_type = other RNA

SEQ ID NO: 74 - RNA, 111 nt, synthetic construct
  source 1..111: mol_type = other RNA
tcaccttggggaaaagtgcgagactccagacaaggggagtctacaacagtaggttcacccgtagggttacccccgcgtcatcctcggaaggcgcggggcgaactctttttt

SEQ ID NO: 75 - RNA, 36 nt, synthetic construct
  source 1..36: mol_type = other RNA
gtcttgagtttgcgcccttccccaaggtgatacgct

SEQ ID NO: 76 - RNA, 106 nt, synthetic construct
  source 1..106: mol_type = other RNA
tcaccttggggaagggcgctgctccagacaagggaagccacttgctggcttacccgtaaagtttcaaccccgcgttgccttcaggcggcgcggggtgaactttttt

SEQ ID NO: 77 - RNA, 111 nt, synthetic construct
  source 1..111: mol_type = other RNA
gttctgctaccatcgaaatttttgctaggctacaagaaattgtagtctagcaaaggttttgatgatctagcagaacaagggtttatcccggaatcgactccttagggagtc

SEQ ID NO: 78 - RNA, 69 nt, synthetic construct
  source 1..69: mol_type = other RNA
gttctgctaccatcgaaagatgatctagcagaacaagggtttatcccggaatcgactccttagggagtc

SEQ ID NO: 79 - RNA, 108 nt, synthetic construct
  source 1..108: mol_type = other RNA
gttttggtaccctctaaatttttgctatactgaaaagtatagcaaaggtttagaggacctatcaaaacaagggaattattcccgaaatcggaactgctaagcagttcc

SEQ ID NO: 80 - RNA, 74 nt, synthetic construct
  source 1..74: mol_type = other RNA
gttttggtaccctcgaaagaggacctatcaaaacaagggaattattcccgaaatcggaactgctaagcagttcc

SEQ ID NO: 81 - RNA, 118 nt, synthetic construct
  source 1..118: mol_type = other RNA
gttttgctaccctcacaatttttgctatgtaagaaattacatagcaaagattgtgaggatctagcgaaacaagggctgtcttaggacatgtcccgaaatcgagtctcgtaggagactc

SEQ ID NO: 82 - RNA, 82 nt, synthetic construct
  source 1..82: mol_type = other RNA
gttttgctaccctcgaaagaggatctagcgaaacaagggctgtcttaggacatgtcccgaaatcgagtctcgtaggagactc

SEQ ID NO: 83 - RNA, 117 nt, synthetic construct
  source 1..117: mol_type = other RNA
gttttagtacctgaaagaattgagttattgtaaaacgaaagttttgcaataactcaattttttcagatctactaaaacaaggctttatgccgaaatcaaggacacagataagtgtcc

SEQ ID NO: 84 - RNA, 73 nt, synthetic construct
  source 1..73: mol_type = other RNA
gttttagtacctgagaaatcagatctactaaaacaaggctttatgccgaaatcaaggacacagataagtgtcc

SEQ ID NO: 85 - RNA, 118 nt, synthetic construct
  source 1..118: mol_type = other RNA
gttttgctaccctcacaatttttgctatgtaagaaattacatagcaaagattgtgaggatctagcaaaacaagggctgtcttaggacatgtcccgaaatcgagtctcgtaggagactc

SEQ ID NO: 86 - RNA, 82 nt, synthetic construct
  source 1..82: mol_type = other RNA
gttttgctaccctcgaaagaggatctagcaaaacaagggctgtcttaggacatgtcccgaaatcgagtctcgtaggagactc

SEQ ID NO: 87 - RNA, 139 nt, synthetic construct
  source 1..139: mol_type = other RNA
gtcttgagctcgcacttttccccaagctgagaaatcaccttggggaaaagtgcgagactccagacaaggggaacctacaacggtaggttcacccgtagggttacccccgcgtcatcttcggaaggcgcggggcgaactc

SEQ ID NO: 88 - RNA, 103 nt, synthetic construct
  source 1..103: mol_type = other RNA
gtcttgagctcggaaacgagactccagacaaggggaacctacaacggtaggttcacccgtagggttacccccgcgtcatcttcggaaggcgcggggcgaactc

SEQ ID NO: 89 - RNA, 139 nt, synthetic construct
  source 1..139: mol_type = other RNA
gtcttgagctcgcacttttccccaagctgagaaatcaccttggggaaaagtgcgagactccagacaaggggagtctacaacagtaggttcacccgtagggttacccccgcgtcatcctcggaaggcgcggggcgaactc

SEQ ID NO: 90 - RNA, 103 nt, synthetic construct
  source 1..103: mol_type = other RNA
gtcttgagctcggaaacgagactccagacaaggggagtctacaacagtaggttcacccgtagggttacccccgcgtcatcctcggaaggcgcggggcgaactc

SEQ ID NO: 91 - RNA, 134 nt, synthetic construct
  source 1..134: mol_type = other RNA
gtcttgagtttgcgcccttccccaaggtgagaaatcaccttggggaagggcgctgctccagacaagggaagccacttgctggcttacccgtaaagtttcaaccccgcgttgccttcaggcggcgcggggtgaac

SEQ ID NO: 92 - RNA, 102 nt, synthetic construct
  source 1..102: mol_type = other RNA
gtcttgagtttgcggaaacgctgctccagacaagggaagccacttgctggcttacccgtaaagtttcaaccccgcgttgccttcaggcggcgcggggtgaac

SEQ ID NO: 93 - RNA, 117 nt, synthetic construct
  source 1..117: mol_type = other RNA
gttctgctaccatcgaaatttttgctaggctacaagaaattgtagtctagcaaaggttttgatgatctagcagaacaagggtttatcccggaatcgactccttagggagtctttttt

SEQ ID NO: 94 - RNA, 75 nt, synthetic construct
  source 1..75: mol_type = other RNA
gttctgctaccatcgaaagatgatctagcagaacaagggtttatcccggaatcgactccttagggagtctttttt

SEQ ID NO: 95 - RNA, 114 nt, synthetic construct
  source 1..114: mol_type = other RNA
gttttggtaccctctaaatttttgctatactgaaaagtatagcaaaggtttagaggacctatcaaaacaagggaattattcccgaaatcggaactgctaagcagttcctttttt

SEQ ID NO: 96 - RNA, 80 nt, synthetic construct
  source 1..80: mol_type = other RNA
gttttggtaccctcgaaagaggacctatcaaaacaagggaattattcccgaaatcggaactgctaagcagttcctttttt

SEQ ID NO: 97 - RNA, 124 nt, synthetic construct
  source 1..124: mol_type = other RNA
gttttgctaccctcacaatttttgctatgtaagaaattacatagcaaagattgtgaggatctagcgaaacaagggctgtcttaggacatgtcccgaaatcgagtctcgtaggagactctttttt

SEQ ID NO: 98 - RNA, 88 nt, synthetic construct
  source 1..88: mol_type = other RNA
gttttgctaccctcgaaagaggatctagcgaaacaagggctgtcttaggacatgtcccgaaatcgagtctcgtaggagactctttttt

SEQ ID NO: 99 - RNA, 123 nt, synthetic construct
  source 1..123: mol_type = other RNA
gttttagtacctgaaagaattgagttattgtaaaacgaaagttttgcaataactcaattttttcagatctactaaaacaaggctttatgccgaaatcaaggacacagataagtgtcctttttt

SEQ ID NO: 100 - RNA, 79 nt, synthetic construct
  source 1..79: mol_type = other RNA
gttttagtacctgagaaatcagatctactaaaacaaggctttatgccgaaatcaaggacacagataagtgtcctttttt

SEQ ID NO: 101 - RNA, 124 nt, synthetic construct
  source 1..124: mol_type = other RNA
gttttgctaccctcacaatttttgctatgtaagaaattacatagcaaagattgtgaggatctagcaaaacaagggctgtcttaggacatgtcccgaaatcgagtctcgtaggagactctttttt

SEQ ID NO: 102 - RNA, 88 nt, synthetic construct
  source 1..88: mol_type = other RNA
gttttgctaccctcgaaagaggatctagcaaaacaagggctgtcttaggacatgtcccgaaatcgagtctcgtaggagactctttttt

SEQ ID NO: 103 - RNA, 145 nt, synthetic construct
  source 1..145: mol_type = other RNA
gtcttgagctcgcacttttccccaagctgagaaatcaccttggggaaaagtgcgagactccagacaaggggaacctacaacggtaggttcacccgtagggttacccccgcgtcatcttcggaaggcgcggggcgaactctttttt

SEQ ID NO: 104 - RNA, 109 nt, synthetic construct
  source 1..109: mol_type = other RNA
gtcttgagctcggaaacgagactccagacaaggggaacctacaacggtaggttcacccgtagggttacccccgcgtcatcttcggaaggcgcggggcgaactctttttt

SEQ ID NO: 105 - RNA, 145 nt, synthetic construct
  source 1..145: mol_type = other RNA
gtcttgagctcgcacttttccccaagctgagaaatcaccttggggaaaagtgcgagactccagacaaggggagtctacaacagtaggttcacccgtagggttacccccgcgtcatcctcggaaggcgcggggcgaactctttttt

SEQ ID NO: 106 - RNA, 109 nt, synthetic construct
  source 1..109: mol_type = other RNA
gtcttgagctcggaaacgagactccagacaaggggagtctacaacagtaggttcacccgtagggttacccccgcgtcatcctcggaaggcgcggggcgaactctttttt

SEQ ID NO: 107 - RNA, 140 nt, synthetic construct
  source 1..140: mol_type = other RNA
gtcttgagtttgcgcccttccccaaggtgagaaatcaccttggggaagggcgctgctccagacaagggaagccacttgctggcttacccgtaaagtttcaaccccgcgttgccttcaggcggcgcggggtgaactttttt

SEQ ID NO: 108 - RNA, 108 nt, synthetic construct
  source 1..108: mol_type = other RNA
gtcttgagtttgcggaaacgctgctccagacaagggaagccacttgctggcttacccgtaaagtttcaaccccgcgttgccttcaggcggcgcggggtgaactttttt

SEQ ID NO: 109 - protein, 18 aa, synthetic construct
KRTADGSEFESPKKKRKV

SEQ ID NO: 110 - protein, 7 aa, synthetic construct
PKKKRKV

SEQ ID NO: 111 - protein, 7 aa, synthetic construct
PKKKRRV

SEQ ID NO: 112 - protein, 16 aa, synthetic construct
KRPAATKKAGQAKKKK

SEQ ID NO: 113 - protein, 11 aa, synthetic construct
YGRKKRRQRRR

SEQ ID NO: 114 - protein, 9 aa, synthetic construct
RKKRRQRRR

SEQ ID NO: 115 - protein, 9 aa, synthetic construct
PAAKRVKLD

SEQ ID NO: 116 - protein, 11 aa, synthetic construct
RQRRNELKRSP

SEQ ID NO: 117 - protein, 8 aa, synthetic construct
VSRKRPRP

SEQ ID NO: 118 - protein, 8 aa, synthetic construct
PPKKARED

SEQ ID NO: 119 - protein, 8 aa, synthetic construct
PQPKKKPL

SEQ ID NO: 120 - protein, 12 aa, synthetic construct
SALIKKKKKMAP

SEQ ID NO: 121 - protein, 7 aa, synthetic construct
PKQKKRK

SEQ ID NO: 122 - protein, 10 aa, synthetic construct
RKLKKKIKKL

SEQ ID NO: 123 - protein, 10 aa, synthetic construct
REKKKFLKRR

SEQ ID NO: 124 - protein, 20 aa, synthetic construct
KRKGDEVDGVDEVAKKKSKK

SEQ ID NO: 125 - protein, 17 aa, synthetic construct
RKCLQAGMNLEARKTKK

SEQ ID NO: 126 - protein, 38 aa, synthetic construct
NQSSNFGPMKGGNFGGRSSGPYGGGGQYFAKPRNQGGY

SEQ ID NO: 127 - protein, 42 aa, synthetic construct
RMRIZFKNKGKDTAELRRRRVEVSVELRKAKKDEQILKRRNV

SEQ ID NO: 128 - protein, 14 aa, synthetic construct
GKPIPNPLLGLDST

SEQ ID NO: 129 - protein, 9 aa, synthetic construct
IPNPLLGLD

SEQ ID NO: 130 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 131 - DNA, 66 bp, synthetic construct
  source 1..66: mol_type = other DNA
cctctaatacgactcactataggtcgtcgccctcgaacttcacctgttttggtaccctcgaaagag

SEQ ID NO: 132 - DNA, 23 bp, synthetic construct
  source 1..23: mol_type = other DNA
aaaaaaggaactgcttagcagtt

SEQ ID NO: 133 - DNA, 66 bp, synthetic construct
  source 1..66: mol_type = other DNA
cctctaatacgactcactataggtcgtcgccctcgaacttcacctgttctgctaccatcgaaagat

SEQ ID NO: 134 - DNA, 22 bp, synthetic construct
  source 1..22: mol_type = other DNA
aaaaaagactccctaaggagtc

SEQ ID NO: 135 - DNA, 60 bp, synthetic construct
  source 1..60: mol_type = other DNA
taatacgactcactataggtcgtcgccctcgaacttcacctgttttgctaccctcgaaag

SEQ ID NO: 136 - DNA, 65 bp, synthetic construct
  source 1..65: mol_type = other DNA
taatacgactcactataggtcgtcgccctcgaacttcacctgttttgctaccctcacaatttttg

SEQ ID NO: 137 - DNA, 21 bp, synthetic construct
  source 1..21: mol_type = other DNA
aaaaagagtctcctacgagac

SEQ ID NO: 138 - DNA, 66 bp, synthetic construct
  source 1..66: mol_type = other DNA
cctctaatacgactcactataggtcgtcgccctcgaacttcacctgttttgctaccctcgaaagag

SEQ ID NO: 139 - DNA, 22 bp, synthetic construct
  source 1..22: mol_type = other DNA
aaaaaagagtctcctacgagac

SEQ ID NO: 140 - DNA, 67 bp, synthetic construct
  source 1..67: mol_type = other DNA
cctctaatacgactcactataggtcgtcgccctcgaacttcacctgttttagtacctgagaaatcag

SEQ ID NO: 141 - DNA, 23 bp, synthetic construct
  source 1..23: mol_type = other DNA
aaaaaaggacacttatctgtgtc

SEQ ID NO: 142 - DNA, 65 bp, synthetic construct
  source 1..65: mol_type = other DNA
cctctaatacgactcactataggtcgtcgccctcgaacttcacctgtcttgagtttgcggaaacg

SEQ ID NO: 143 - DNA, 19 bp, synthetic construct
  source 1..19: mol_type = other DNA
aaaaaagttcaccccgcgc

SEQ ID NO: 144 - DNA, 64 bp, synthetic construct
  source 1..64: mol_type = other DNA
cctctaatacgactcactataggtcgtcgccctcgaacttcacctgtcttgagctcggaaacga

SEQ ID NO: 145 - DNA, 19 bp, synthetic construct
  source 1..19: mol_type = other DNA
aaaaaagagttcgccccgc

SEQ ID NO: 146 - DNA, 31 bp, synthetic construct
  source 1..31: mol_type = other DNA
cggcattcctgctgaaccgctcttccgatct

SEQ ID NO: 147 - DNA, 30 bp, synthetic construct
  source 1..30: mol_type = other DNA
gatcggaagagcggttcagcaggaatgccg

SEQ ID NO: 148 - DNA, 55 bp, synthetic construct
  source 1..55: mol_type = other DNA
tcgtcggcagcgtcagatgtgtataagagacagctgctgaaccgctcttccgatc

SEQ ID NO: 149 - DNA, 60 bp, synthetic construct
  source 1..60: mol_type = other DNA
tcgtcggcagcgtcagatgtgtataagagacagtaagactgctgaaccgctcttccgatc

SEQ ID NO: 150 - DNA, 72 bp, synthetic construct
  source 1..72: mol_type = other DNA
tcgtcggcagcgtcagatgtgtataagagacaggctagacctaatgtgatctgctgaaccgctcttccgatc

SEQ ID NO: 151 - DNA, 63 bp, synthetic construct
  source 1..63: mol_type = other DNA
gtctcgtgggctcggagatgtgtataagagacagtctgcgttctgatttaatctgtatcaggc

SEQ ID NO: 152 - DNA, 23 bp, synthetic construct
  source 1..23: mol_type = other DNA
gctggacggcgacgtaaacggcc

SEQ ID NO: 153 - DNA, 23 bp, synthetic construct
  source 1..23: mol_type = other DNA
gggcgagggcgatgccacctacg

SEQ ID NO: 154 - DNA, 23 bp, synthetic construct
  source 1..23: mol_type = other DNA
cctgaagttcatctgcaccaccg

SEQ ID NO: 155 - DNA, 23 bp, synthetic construct
  source 1..23: mol_type = other DNA
gtcagggtggtcacgagggtggg

SEQ ID NO: 156 - DNA, 23 bp, synthetic construct
  source 1..23: mol_type = other DNA
ccacatgaagcagcacgacttct

SEQ ID NO: 157 - DNA, 23 bp, synthetic construct
  source 1..23: mol_type = other DNA
cttcttcaaggacgacggcaact

SEQ ID NO: 158 - DNA, 23 bp, synthetic construct
  source 1..23: mol_type = other DNA
gtttacgtcgccgtccagctcga

SEQ ID NO: 159 - DNA, 23 bp, synthetic construct
  source 1..23: mol_type = other DNA
tccgccatgcccgaaggctacgt

SEQ ID NO: 160 - DNA, 23 bp, synthetic construct
  source 1..23: mol_type = other DNA
tttacgtcgccgtccagctcgac

SEQ ID NO: 161 - DNA, 23 bp, synthetic construct
  source 1..23: mol_type = other DNA
gaccctgaagttcatctgcacca

SEQ ID NO: 162 - DNA, 23 bp, synthetic construct
  source 1..23: mol_type = other DNA
aagtcgtgctgcttcatgtggtc

SEQ ID NO: 163 - DNA, 23 bp, synthetic construct
  source 1..23: mol_type = other DNA
ggcaagctgaccctgaagttcat

SEQ ID NO: 164 - DNA, 23 bp, synthetic construct
  source 1..23: mol_type = other DNA
gcactgcacgccgtaggtcaggg

SEQ ID NO: 165 - DNA, 23 bp, synthetic construct
  source 1..23: mol_type = other DNA
atgcccgaaggctacgtccagga

SEQ ID NO: 166 - DNA, 24 bp, synthetic construct
  source 1..24: mol_type = other DNA
gcccatcctggtcgagctggacgg

SEQ ID NO: 167 - DNA, 23 bp, synthetic construct
  source 1..23: mol_type = other DNA
ggggtagcggctgaagcactgca

SEQ ID NO: 168 - DNA, 24 bp, synthetic construct
  source 1..24: mol_type = other DNA
gtagttgccgtcgtccttgaagaa

SEQ ID NO: 169 - DNA, 23 bp, synthetic construct
  source 1..23: mol_type = other DNA
ggcgaggagctgttcaccggggt

SEQ ID NO: 170 - DNA, 23 bp, synthetic construct
  source 1..23: mol_type = other DNA
cctggcccaccctcgtgaccacc

SEQ ID NO: 171 - DNA, 23 bp, synthetic construct
  source 1..23: mol_type = other DNA
agcacgacttcttcaagtccgcc

SEQ ID NO: 172 - DNA, 23 bp, synthetic construct
  source 1..23: mol_type = other DNA
gcccatcctggtcgagctggacg

SEQ ID NO: 173 - DNA, 23 bp, synthetic construct
  source 1..23: mol_type = other DNA
tgcagatgaacttcagggtcagc

SEQ ID NO: 174 - DNA, 23 bp, synthetic construct
  source 1..23: mol_type = other DNA
gcacgacttcttcaagtccgcca

SEQ ID NO: 175 - DNA, 23 bp, synthetic construct
  source 1..23: mol_type = other DNA
cgccctcgccctcgccggacacg

SEQ ID NO: 176 - DNA, 22 bp, synthetic construct
  source 1..22: mol_type = other DNA
gccctcgccctcgccggacacg

SEQ ID NO: 177 - DNA, 23 bp, synthetic construct
  source 1..23: mol_type = other DNA
gcttcatgtggtcggggtagcgg

SEQ ID NO: 178 - DNA, 21 bp, synthetic construct
  source 1..21: mol_type = other DNA
ttcatgtggtcggggtagcgg

SEQ ID NO: 179 - DNA, 23 bp, synthetic construct
  source 1..23: mol_type = other DNA
gcaactacaagacccgcgccgag

SEQ ID NO: 180 - DNA, 21 bp, synthetic construct
  source 1..21: mol_type = other DNA
aactacaagacccgcgccgag

SEQ ID NO: 181 - DNA, 21 bp, synthetic construct
  source 1..21: mol_type = other DNA
ttacgtcgccgtccagctcga

SEQ ID NO: 182 - DNA, 24 bp, synthetic construct
  source 1..24: mol_type = other DNA
gtagcggctgaagcactgcacgcc

SEQ ID NO: 183 - DNA, 21 bp, synthetic construct
  source 1..21: mol_type = other DNA
gcggctgaagcactgcacgcc

SEQ ID NO: 184 - DNA, 21 bp, synthetic construct
  source 1..21: mol_type = other DNA
cagggtggtcacgagggtggg

SEQ ID NO: 185 - DNA, 34 bp, synthetic construct
  source 1..34: mol_type = other DNA
cgagctggacggcgacgtaaacggccacaagttc

SEQ ID NO: 186 - DNA, 34 bp, synthetic construct
  source 1..34: mol_type = other DNA
cgagggcgagggcgatgccacctacggcaagctg

SEQ ID NO: 187 - DNA, 34 bp, synthetic construct
  source 1..34: mol_type = other DNA
gaccctgaagttcatctgcaccaccggcaagctg

SEQ ID NO: 188 - DNA, 36 bp, synthetic construct
  source 1..36: mol_type = other DNA
taggtcagggtggtcacgagggtgggccagggcacg

SEQ ID NO: 189 - DNA, 36 bp, synthetic construct
  source 1..36: mol_type = other DNA
cgaccacatgaagcagcacgacttcttcaagtccgc

SEQ ID NO: 190 - DNA, 36 bp, synthetic construct
  source 1..36: mol_type = other DNA
catcttcttcaaggacgacggcaactacaagacccg

SEQ ID NO: 191 - DNA, 34 bp, synthetic construct
  source 1..34: mol_type = other DNA
gccgtttacgtcgccgtccagctcgaccaggatg

SEQ ID NO: 192 - DNA, 34 bp, synthetic construct
  source 1..34: mol_type = other DNA
taggtcagggtggtcacgagggtgggccagggca

SEQ ID NO: 193 - DNA, 34 bp, synthetic construct
  source 1..34: mol_type = other DNA
aagtccgccatgcccgaaggctacgtccaggagc

SEQ ID NO: 194 - DNA, 35 bp, synthetic construct
  source 1..35: mol_type = other DNA
ccgtttacgtcgccgtccagctcgaccaggatggg

SEQ ID NO: 195 - DNA, 35 bp, synthetic construct
  source 1..35: mol_type = other DNA
gctgaccctgaagttcatctgcaccaccggcaagc

SEQ ID NO: 196 - DNA, 35 bp, synthetic construct
  source 1..35: mol_type = other DNA
aagaagtcgtgctgcttcatgtggtcggggtagcg

SEQ ID NO: 197 - DNA, 37 bp, synthetic construct
  source 1..37: mol_type = other DNA
tacggcaagctgaccctgaagttcatctgcaccaccg

SEQ ID NO: 198 - DNA, 37 bp, synthetic construct
  source 1..37: mol_type = other DNA
gaagcactgcacgccgtaggtcagggtggtcacgagg

SEQ ID NO: 199 - DNA, 37 bp, synthetic construct
  source 1..37: mol_type = other DNA
gccatgcccgaaggctacgtccaggagcgcaccatct

SEQ ID NO: 200 - DNA, 36 bp, synthetic construct
  source 1..36: mol_type = other DNA
ggtgcccatcctggtcgagctggacggcgacgtaaa

SEQ ID NO: 201 - DNA, 35 bp, synthetic construct
  source 1..35: mol_type = other DNA
gtcggggtagcggctgaagcactgcacgccgtagg

SEQ ID NO: 202 - DNA, 36 bp, synthetic construct
  source 1..36: mol_type = other DNA
cttgtagttgccgtcgtccttgaagaagatggtgcg

SEQ ID NO: 203 - DNA, 37 bp, synthetic construct
  source 1..37: mol_type = other DNA
aagggcgaggagctgttcaccggggtggtgcccatcc

SEQ ID NO: 204 - DNA, 37 bp, synthetic construct
  source 1..37: mol_type = other DNA
tgccctggcccaccctcgtgaccaccctgacctacgg

SEQ ID NO: 205 - DNA, 37 bp, synthetic construct
  source 1..37: mol_type = other DNA
agcagcacgacttcttcaagtccgccatgcccgaagg

SEQ ID NO: 206 - DNA, 37 bp, synthetic construct
  source 1..37: mol_type = other DNA
ggtgcccatcctggtcgagctggacggcgacgtaaac

SEQ ID NO: 207 - DNA, 37 bp, synthetic construct
  source 1..37: mol_type = other DNA
tggtgcagatgaacttcagggtcagcttgccgtaggt

SEQ ID NO: 208 - DNA, 37 bp, synthetic construct
  source 1..37: mol_type = other DNA
gcagcacgacttcttcaagtccgccatgcccgaaggc

SEQ ID NO: 209 - DNA, 34 bp, synthetic construct
  source 1..34: mol_type = other DNA
catcgccctcgccctcgccggacacgctgaactt

SEQ ID NO: 210 - DNA, 33 bp, synthetic construct
  source 1..33: mol_type = other DNA
atcgccctcgccctcgccggacacgctgaactt

SEQ ID NO: 211 - DNA, 34 bp, synthetic construct
  source 1..34: mol_type = other DNA
gctgcttcatgtggtcggggtagcggctgaagca

SEQ ID NO: 212 - DNA, 32 bp, synthetic construct
  source 1..32: mol_type = other DNA
tgcttcatgtggtcggggtagcggctgaagca

SEQ ID NO: 213 - DNA, 34 bp, synthetic construct
  source 1..34: mol_type = other DNA
acggcaactacaagacccgcgccgaggtgaagtt

SEQ ID NO: 214 - DNA, 32 bp, synthetic construct
  source 1..32: mol_type = other DNA
ggcaactacaagacccgcgccgaggtgaagtt

SEQ ID NO: 215 - DNA, 36 bp, synthetic construct
  source 1..36: mol_type = other DNA
gccgtttacgtcgccgtccagctcgaccaggatggg

SEQ ID NO: 216 - DNA, 34 bp, synthetic construct
  source 1..34: mol_type = other DNA
cgtttacgtcgccgtccagctcgaccaggatggg

SEQ ID NO: 217 - DNA, 37 bp, synthetic construct
  source 1..37: mol_type = other DNA
ggggtagcggctgaagcactgcacgccgtaggtcagg

SEQ ID NO: 218 - DNA, 34 bp, synthetic construct
  source 1..34: mol_type = other DNA
gtagcggctgaagcactgcacgccgtaggtcagg

SEQ ID NO: 219 - DNA, 34 bp, synthetic construct
  source 1..34: mol_type = other DNA
ggtcagggtggtcacgagggtgggccagggcacg

SEQ ID NO: 220 - DNA, 27 bp, synthetic construct
  source 1..27: mol_type = other DNA
caccgctggacggcgacgtaaacggcc

SEQ ID NO: 221 - DNA, 27 bp, synthetic construct
  source 1..27: mol_type = other DNA
caccgggcgagggcgatgccacctacg

SEQ ID NO: 222 - DNA, 28 bp, synthetic construct
  source 1..28: mol_type = other DNA
caccgcctgaagttcatctgcaccaccg

SEQ ID NO: 223 - DNA, 27 bp, synthetic construct
  source 1..27: mol_type = other DNA
caccgtcagggtggtcacgagggtggg

SEQ ID NO: 224 - DNA, 28 bp, synthetic construct
  source 1..28: mol_type = other DNA
caccgccacatgaagcagcacgacttct

SEQ ID NO: 225 - DNA, 28 bp, synthetic construct
  source 1..28: mol_type = other DNA
caccgcttcttcaaggacgacggcaact

SEQ ID NO: 226 - DNA, 27 bp, synthetic construct
  source 1..27: mol_type = other DNA
caccgtttacgtcgccgtccagctcga

SEQ ID NO: 227 - DNA, 28 bp, synthetic construct
  source 1..28: mol_type = other DNA
caccgtccgccatgcccgaaggctacgt

SEQ ID NO: 228 - DNA, 28 bp, synthetic construct
  source 1..28: mol_type = other DNA
caccgtttacgtcgccgtccagctcgac

SEQ ID NO: 229 - DNA, 27 bp, synthetic construct
  source 1..27: mol_type = other DNA
caccgaccctgaagttcatctgcacca

SEQ ID NO: 230 - DNA, 28 bp, synthetic construct
  source 1..28: mol_type = other DNA
caccgaagtcgtgctgcttcatgtggtc

SEQ ID NO: 231 - DNA, 27 bp, synthetic construct
  source 1..27: mol_type = other DNA
caccggcaagctgaccctgaagttcat

SEQ ID NO: 232 - DNA, 27 bp, synthetic construct
  source 1..27: mol_type = other DNA
caccgcactgcacgccgtaggtcaggg

SEQ ID NO: 233 - DNA, 28 bp, synthetic construct
  source 1..28: mol_type = other DNA
caccgatgcccgaaggctacgtccagga

SEQ ID NO: 234 - DNA, 28 bp, synthetic construct
  source 1..28: mol_type = other DNA
caccgcccatcctggtcgagctggacgg

SEQ ID NO: 235 - DNA, 27 bp, synthetic construct
  source 1..27: mol_type = other DNA
caccggggtagcggctgaagcactgca

SEQ ID NO: 236 - DNA, 28 bp, synthetic construct
  source 1..28: mol_type = other DNA
caccgtagttgccgtcgtccttgaagaa

SEQ ID NO: 237 - DNA, 27 bp, synthetic construct
  source 1..27: mol_type = other DNA
caccggcgaggagctgttcaccggggt

SEQ ID NO: 238 - DNA, 28 bp, synthetic construct
  source 1..28: mol_type = other DNA
caccgcctggcccaccctcgtgaccacc

SEQ ID NO: 239 - DNA, 28 bp, synthetic construct
  source 1..28: mol_type = other DNA
caccgagcacgacttcttcaagtccgcc

SEQ ID NO: 240 - DNA, 27 bp, synthetic construct
  source 1..27: mol_type = other DNA
caccgcccatcctggtcgagctggacg

SEQ ID NO: 241 - DNA, 28 bp, synthetic construct
  source 1..28: mol_type = other DNA
caccgtgcagatgaacttcagggtcagc

SEQ ID NO: 242 - DNA, 27 bp, synthetic construct
  source 1..27: mol_type = other DNA
caccgcacgacttcttcaagtccgcca

SEQ ID NO: 243 - DNA, 28 bp, synthetic construct
  source 1..28: mol_type = other DNA
caccgcgccctcgccctcgccggacacg

SEQ ID NO: 244 - DNA, 26 bp, synthetic construct
  source 1..26: mol_type = other DNA
caccgccctcgccctcgccggacacg

SEQ ID NO: 245 - DNA, 27 bp, synthetic construct
  source 1..27: mol_type = other DNA
caccgcttcatgtggtcggggtagcgg

SEQ ID NO: 246 - DNA, 26 bp, synthetic construct
  source 1..26: mol_type = other DNA
caccgttcatgtggtcggggtagcgg

SEQ ID NO: 247 - DNA, 27 bp, synthetic construct
  source 1..27: mol_type = other DNA
caccgcaactacaagacccgcgccgag

SEQ ID NO: 248 - DNA, 26 bp, synthetic construct
  source 1..26: mol_type = other DNA
caccgaactacaagacccgcgccgag

SEQ ID NO: 249 - DNA, 26 bp, synthetic construct
  source 1..26: mol_type = other DNA
caccgttacgtcgccgtccagctcga

SEQ ID NO: 250 - DNA, 28 bp, synthetic construct
  source 1..28: mol_type = other DNA
caccgtagcggctgaagcactgcacgcc

SEQ ID NO: 251 - DNA, 25 bp, synthetic construct
  source 1..25: mol_type = other DNA
caccgcggctgaagcactgcacgcc

SEQ ID NO: 252 - DNA, 26 bp, synthetic construct
  source 1..26: mol_type = other DNA
caccgcagggtggtcacgagggtggg

SEQ ID NO: 253 - DNA, 27 bp, synthetic construct
  source 1..27: mol_type = other DNA
aaacggccgtttacgtcgccgtccagc

SEQ ID NO: 254 - DNA, 27 bp, synthetic construct
  source 1..27: mol_type = other DNA
aaaccgtaggtggcatcgccctcgccc

SEQ ID NO: 255 - DNA, 28 bp, synthetic construct
  source 1..28: mol_type = other DNA
aaaccggtggtgcagatgaacttcaggc

SEQ ID NO: 256 - DNA, 27 bp, synthetic construct
  source 1..27: mol_type = other DNA
gaaccccaccctcgtgaccaccctgac

SEQ ID NO: 257 - DNA, 28 bp, synthetic construct
  source 1..28: mol_type = other DNA
gaacagaagtcgtgctgcttcatgtggc

SEQ ID NO: 258 - DNA, 28 bp, synthetic construct
  source 1..28: mol_type = other DNA
gaacagttgccgtcgtccttgaagaagc

SEQ ID NO: 259 - DNA, 27 bp, synthetic construct
  source 1..27: mol_type = other DNA
aaactcgagctggacggcgacgtaaac

SEQ ID NO: 260 - DNA, 27 bp, synthetic construct
  source 1..27: mol_type = other DNA
aaaccccaccctcgtgaccaccctgac

SEQ ID NO: 261 - DNA, 28 bp, synthetic construct
  source 1..28: mol_type = other DNA
aaacacgtagccttcgggcatggcggac

SEQ ID NO: 262 - DNA, 28 bp, synthetic construct
  source 1..28: mol_type = other DNA
aaacgtcgagctggacggcgacgtaaac

SEQ ID NO: 263 - DNA, 27 bp, synthetic construct
  source 1..27: mol_type = other DNA
aaactggtgcagatgaacttcagggtc

SEQ ID NO: 264 - DNA, 28 bp, synthetic construct
  source 1..28: mol_type = other DNA
aaacgaccacatgaagcagcacgacttc

SEQ ID NO: 265 - DNA, 27 bp, synthetic construct
  source 1..27: mol_type = other DNA
aaacatgaacttcagggtcagcttgcc

SEQ ID NO: 266 - DNA, 27 bp, synthetic construct
  source 1..27: mol_type = other DNA
aaacccctgacctacggcgtgcagtgc

SEQ ID NO: 267 - DNA, 28 bp, synthetic construct
  source 1..28: mol_type = other DNA
aaactcctggacgtagccttcgggcatc

SEQ ID NO: 268 - DNA, 28 bp, synthetic construct
  source 1..28: mol_type = other DNA
agacccgtccagctcgaccaggatgggc

SEQ ID NO: 269 - DNA, 27 bp, synthetic construct
  source 1..27: mol_type = other DNA
agactgcagtgcttcagccgctacccc

SEQ ID NO: 270 - DNA, 28 bp, synthetic construct
  source 1..28: mol_type = other DNA
agacttcttcaaggacgacggcaactac

SEQ ID NO: 271 - DNA, 27 bp, synthetic construct
  source 1..27: mol_type = other DNA
agacaccccggtgaacagctcctcgcc

SEQ ID NO: 272 - DNA, 28 bp, synthetic construct
  source 1..28: mol_type = other DNA
agacggtggtcacgagggtgggccaggc

SEQ ID NO: 273 - DNA, 28 bp, synthetic construct
  source 1..28: mol_type = other DNA
agacggcggacttgaagaagtcgtgctc

SEQ ID NO: 274 - DNA, 27 bp, synthetic construct
  source 1..27: mol_type = other DNA
agaccgtccagctcgaccaggatgggc

SEQ ID NO: 275 - DNA, 28 bp, synthetic construct
  source 1..28: mol_type = other DNA
agacgctgaccctgaagttcatctgcac

SEQ ID NO: 276 - DNA, 27 bp, synthetic construct
  source 1..27: mol_type = other DNA
agactggcggacttgaagaagtcgtgc

SEQ ID NO: 277 - DNA, 28 bp, synthetic construct
  source 1..28: mol_type = other DNA
aaaccgtgtccggcgagggcgagggcgc

SEQ ID NO: 278 - DNA, 26 bp, synthetic construct
  source 1..26: mol_type = other DNA
aaaccgtgtccggcgagggcgagggc

SEQ ID NO: 279 - DNA, 27 bp, synthetic construct
  source 1..27: mol_type = other DNA
aaacccgctaccccgaccacatgaagc

SEQ ID NO: 280 - DNA, 26 bp, synthetic construct
  source 1..26: mol_type = other DNA
aaacccgctaccccgaccacatgaac

SEQ ID NO: 281 - DNA, 27 bp, synthetic construct
  source 1..27: mol_type = other DNA
aaacctcggcgcgggtcttgtagttgc

SEQ ID NO: 282 - DNA, 26 bp, synthetic construct
  source 1..26: mol_type = other DNA
aaacctcggcgcgggtcttgtagttc

SEQ ID NO: 283 - DNA, 26 bp, synthetic construct
  source 1..26: mol_type = other DNA
aaactcgagctggacggcgacgtaac

SEQ ID NO: 284 - DNA, 28 bp, synthetic construct
  source 1..28: mol_type = other DNA
aaacggcgtgcagtgcttcagccgctac

SEQ ID NO: 285 - DNA, 25 bp, synthetic construct
  source 1..25: mol_type = other DNA
aaacggcgtgcagtgcttcagccgc

SEQ ID NO: 286 - DNA, 26 bp, synthetic construct
  source 1..26: mol_type = other DNA
aaaccccaccctcgtgaccaccctgc

SEQ ID NO: 287 - RNA, 23 nt, synthetic construct
  source 1..23: mol_type = other RNA
ttgtggctgacccgyggctgctc

SEQ ID NO: 288 - RNA, 23 nt, synthetic construct
  source 1..23: mol_type = other RNA
ttgtggctgacccgtggctgctc

SEQ ID NO: 289 - RNA, 23 nt, synthetic construct
  source 1..23: mol_type = other RNA
ttgtggctgacccgcggctgctc

SEQ ID NO: 290 - RNA, 23 nt, synthetic construct
  source 1..23: mol_type = other RNA
cttgtggctgacccgyggctgct

SEQ ID NO: 291 - RNA, 23 nt, synthetic construct
  source 1..23: mol_type = other RNA
cttgtggctgacccgtggctgct

SEQ ID NO: 292 - RNA, 23 nt, synthetic construct
  source 1..23: mol_type = other RNA
cttgtggctgacccgcggctgct

SEQ ID NO: 293 - RNA, 23 nt, synthetic construct
  source 1..23: mol_type = other RNA
ggcccttgtggctgacccgyggc

SEQ ID NO: 294 - RNA, 23 nt, synthetic construct
  source 1..23: mol_type = other RNA
ggcccttgtggctgacccgtggc

SEQ ID NO: 295 - RNA, 23 nt, synthetic construct
  source 1..23: mol_type = other RNA
ggcccttgtggctgacccgcggc

SEQ ID NO: 296 - RNA, 23 nt, synthetic construct
  source 1..23: mol_type = other RNA
cttgggtgggagcagccrcgggt

SEQ ID NO: 297 - RNA, 23 nt, synthetic construct
  source 1..23: mol_type = other RNA
cttgggtgggagcagccacgggt

SEQ ID NO: 298 - RNA, 23 nt, synthetic construct
  source 1..23: mol_type = other RNA
cttgggtgggagcagccgcgggt

SEQ ID NO: 299 - RNA, 23 nt, synthetic construct
  source 1..23: mol_type = other RNA
gagcagccrcgggtcagccacaa

SEQ ID NO: 300 - RNA, 23 nt, synthetic construct
  source 1..23: mol_type = other RNA
gagcagccacgggtcagccacaa

SEQ ID NO: 301 - RNA, 23 nt, synthetic construct
  source 1..23: mol_type = other RNA
gagcagccgcgggtcagccacaa

SEQ ID NO: 302 - RNA, 23 nt, synthetic construct
  source 1..23: mol_type = other RNA
catggctgtggcccttgtggctg

SEQ ID NO: 303 - RNA, 23 nt, synthetic construct
  source 1..23: mol_type = other RNA
gtgggagcagccrcgggtcagcc

SEQ ID NO: 304 - RNA, 23 nt, synthetic construct
  source 1..23: mol_type = other RNA
gtgggagcagccacgggtcagcc

SEQ ID NO: 305 - RNA, 23 nt, synthetic construct
  source 1..23: mol_type = other RNA
gtgggagcagccgcgggtcagcc

SEQ ID NO: 306 - RNA, 23 nt, synthetic construct
  source 1..23: mol_type = other RNA
ggctgacccgyggctgctcccac

SEQ ID NO: 307 - RNA, 23 nt, synthetic construct
  source 1..23: mol_type = other RNA
ggctgacccgtggctgctcccac

SEQ ID NO: 308 - RNA, 23 nt, synthetic construct
  source 1..23: mol_type = other RNA
ggctgacccgcggctgctcccac

SEQ ID NO: 309 - RNA, 23 nt, synthetic construct
  source 1..23: mol_type = other RNA
cagaaagagagagtagcgcgagc

SEQ ID NO: 310 - RNA, 23 nt, synthetic construct
  source 1..23: mol_type = other RNA
cacgtcatccagcagagaatgga

SEQ ID NO: 311 - RNA, 23 nt, synthetic construct
  source 1..23: mol_type = other RNA
cattcttcagtaagtcaacttca

SEQ ID NO: 312 - RNA, 23 nt, synthetic construct
  source 1..23: mol_type = other RNA
agcattcgggccgagatgtctcg

SEQ ID NO: 313 - RNA, 23 nt, synthetic construct
  source 1..23: mol_type = other RNA
gagatgtctcgctccgtggcctt

SEQ ID NO: 314 - RNA, 23 nt, synthetic construct
  source 1..23: mol_type = other RNA
ggatagcctccaggccagaaaga

SEQ ID NO: 315 - RNA, 23 nt, synthetic construct
  source 1..23: mol_type = other RNA
ttgactttccattctctgctgga

SEQ ID NO: 316 - RNA, 23 nt, synthetic construct
  source 1..23: mol_type = other RNA
ggaaagtcaaatttcctgaattg

SEQ ID NO: 317 - RNA, 23 nt, synthetic construct
  source 1..23: mol_type = other RNA
tttgtctgtgatatacacatcag

SEQ ID NO: 318 - RNA, 24 nt, synthetic construct
  source 1..24: mol_type = other RNA
gtaaggattctgatgtgtatatca

SEQ ID NO: 319 - RNA, 23 nt, synthetic construct
  source 1..23: mol_type = other RNA
ggccacagcactgttgctcttga

SEQ ID NO: 320 - RNA, 23 nt, synthetic construct
  source 1..23: mol_type = other RNA
gagcaacagtgctgtggcctgga

SEQ ID NO: 321 - RNA, 23 nt, synthetic construct
  source 1..23: mol_type = other RNA
ggagcaacaaatctgactttgca

SEQ ID NO: 322 - RNA, 23 nt, synthetic construct
  source 1..23: mol_type = other RNA
aatgtgtcacaaagtaaggattc

SEQ ID NO: 323 - RNA, 23 nt, synthetic construct
  source 1..23: mol_type = other RNA
tctgatgtgtatatcacagacaa

SEQ ID NO: 324 - RNA, 24 nt, synthetic construct
  source 1..24: mol_type = other RNA
gcctggagcaacaaatctgacttt

SEQ ID NO: 325 - RNA, 23 nt, synthetic construct
  source 1..23: mol_type = other RNA
ggcgtttgcacatgcaaagtcag

SEQ ID NO: 326 - RNA, 23 nt, synthetic construct
  source 1..23: mol_type = other RNA
gaagaaggtgtcttctggaataa

SEQ ID NO: 327 - RNA, 24 nt, synthetic construct
  source 1..24: mol_type = other RNA
gctgcccttacctgggctggggaa

SEQ ID NO: 328 - RNA, 23 nt, synthetic construct
  source 1..23: mol_type = other RNA
tgggctggcggccaggatggttc

SEQ ID NO: 329 - RNA, 23 nt, synthetic construct
  source 1..23: mol_type = other RNA
ccgcccagacgactggccagggc

SEQ ID NO: 330 - RNA, 23 nt, synthetic construct
  source 1..23: mol_type = other RNA
caggcgccctggccagtcgtctg

SEQ ID NO: 331 - RNA, 23 nt, synthetic construct
  source 1..23: mol_type = other RNA
agaaccatcctggccgccagccc

SEQ ID NO: 332 - RNA, 23 nt, synthetic construct
  source 1..23: mol_type = other RNA
ctaagaaccatcctggccgccag

SEQ ID NO: 333 - RNA, 24 nt, synthetic construct
  source 1..24: mol_type = other RNA
gctggcggccaggatggttcttag

SEQ ID NO: 334 - RNA, 23 nt, synthetic construct
  source 1..23: mol_type = other RNA
acaactgggctggcggccaggat

SEQ ID NO: 335 - RNA, 23 nt, synthetic construct
  source 1..23: mol_type = other RNA
gtggggctgctccaggcatgcag

SEQ ID NO: 336 - RNA, 23 nt, synthetic construct
  source 1..23: mol_type = other RNA
ggccgccagcccagttgtagcac

SEQ ID NO: 337 - DNA, 23 bp, synthetic construct
  source 1..23: mol_type = other DNA
ttgtggctgacccgtggctgctc

SEQ ID NO: 338 - DNA, 23 bp, synthetic construct
  source 1..23: mol_type = other DNA
cttgtggctgacccgtggctgct

SEQ ID NO: 339 - DNA, 23 bp, synthetic construct
  source 1..23: mol_type = other DNA
ggcccttgtggctgacccgtggc

SEQ ID NO: 340 - DNA, 23 bp, synthetic construct
  source 1..23: mol_type = other DNA
cttgggtgggagcagccacgggt

SEQ ID NO: 341 - DNA, 23 bp, synthetic construct
  source 1..23: mol_type = other DNA
gagcagccacgggtcagccacaa

SEQ ID NO: 342 - DNA, 23 bp, synthetic construct
  source 1..23: mol_type = other DNA
catggctgtggcccttgtggctg

SEQ ID NO: 343 - DNA, 23 bp, synthetic construct
  source 1..23: mol_type = other DNA
gtgggagcagccacgggtcagcc

SEQ ID NO: 344 - DNA, 23 bp, synthetic construct
  source 1..23: mol_type = other DNA
ggctgacccgtggctgctcccac

SEQ ID NO: 345 - DNA, 28 bp, synthetic construct
  source 1..28: mol_type = other DNA
caccgttgtggctgacccgtggctgctc

SEQ ID NO: 346 - DNA, 28 bp, synthetic construct
  source 1..28: mol_type = other DNA
caccgcttgtggctgacccgtggctgct

SEQ ID NO: 347 - DNA, 27 bp, synthetic construct
  source 1..27: mol_type = other DNA
caccggcccttgtggctgacccgtggc

SEQ ID NO: 348 - DNA, 28 bp, synthetic construct
  source 1..28: mol_type = other DNA
caccgcttgggtgggagcagccacgggt

SEQ ID NO: 349 - DNA, 27 bp, synthetic construct
  source 1..27: mol_type = other DNA
caccgagcagccacgggtcagccacaa

SEQ ID NO: 350 - DNA, 28 bp, synthetic construct
  source 1..28: mol_type = other DNA
caccgcatggctgtggcccttgtggctg

SEQ ID NO: 351 - DNA, 27 bp, synthetic construct
  source 1..27: mol_type = other DNA
caccgtgggagcagccacgggtcagcc

SEQ ID NO: 352 - DNA, 27 bp, synthetic construct
  source 1..27: mol_type = other DNA
caccggctgacccgtggctgctcccac

SEQ ID NO: 353 - DNA, 28 bp, synthetic construct
  source 1..28: mol_type = other DNA
agacgagcagccacgggtcagccacaac

SEQ ID NO: 354 - DNA, 28 bp, synthetic construct
  source 1..28: mol_type = other DNA
agacagcagccacgggtcagccacaagc

SEQ ID NO: 355 - DNA, 27 bp, synthetic construct
  source 1..27: mol_type = other DNA
agacgccacgggtcagccacaagggcc

SEQ ID NO: 356 - DNA, 28 bp, synthetic construct
  source 1..28: mol_type = other DNA
agacacccgtggctgctcccacccaagc

SEQ ID NO: 357 - DNA, 27 bp, synthetic construct
  source 1..27: mol_type = other DNA
agacttgtggctgacccgtggctgctc

SEQ ID NO: 358 - DNA, 28 bp, synthetic construct
  source 1..28: mol_type = other DNA
agaccagccacaagggccacagccatgc

SEQ ID NO: 359 - DNA, 27 bp, synthetic construct
  source 1..27: mol_type = other DNA
gaacggctgacccgtggctgctcccac

SEQ ID NO: 360 - DNA, 27 bp, synthetic construct
  source 1..27: mol_type = other DNA
gaacgtgggagcagccacgggtcagcc

SEQ ID NO: 361 - DNA, 20 bp, synthetic construct
  source 1..20: mol_type = other DNA
gggaacgaacaggtaagggg

SEQ ID NO: 362 - DNA, 20 bp, synthetic construct
  source 1..20: mol_type = other DNA
agcgtgaggaagttgatggg

SEQ ID NO: 363 - DNA, 23 bp, synthetic construct
  source 1..23: mol_type = other DNA
cagaaagagagagtagcgcgagc

SEQ ID NO: 364 - DNA, 23 bp, synthetic construct
  source 1..23: mol_type = other DNA
cacgtcatccagcagagaatgga

SEQ ID NO: 365 - DNA, 23 bp, synthetic construct
  source 1..23: mol_type = other DNA
cattcttcagtaagtcaacttca

SEQ ID NO: 366 - DNA, 23 bp, synthetic construct
  source 1..23: mol_type = other DNA
agcattcgggccgagatgtctcg

SEQ ID NO: 367 - DNA, 23 bp, synthetic construct
  source 1..23: mol_type = other DNA
gagatgtctcgctccgtggcctt

SEQ ID NO: 368 - DNA, 23 bp, synthetic construct
  source 1..23: mol_type = other DNA
ggatagcctccaggccagaaaga

SEQ ID NO: 369 - DNA, 23 bp, synthetic construct
  source 1..23: mol_type = other DNA
ttgactttccattctctgctgga

SEQ ID NO: 370 - DNA, 23 bp, synthetic construct
  source 1..23: mol_type = other DNA
ggaaagtcaaatttcctgaattg

SEQ ID NO: 371 - DNA, 23 bp, synthetic construct
  source 1..23: mol_type = other DNA
tttgtctgtgatatacacatcag

SEQ ID NO: 372 - DNA, 24 bp, synthetic construct
  source 1..24: mol_type = other DNA
gtaaggattctgatgtgtatatca

SEQ ID NO: 373 - DNA, 23 bp, synthetic construct
  source 1..23: mol_type = other DNA
ggccacagcactgttgctcttga

SEQ ID NO: 374 - DNA, 23 bp, synthetic construct
  source 1..23: mol_type = other DNA
gagcaacagtgctgtggcctgga

SEQ ID NO: 375 - DNA, 23 bp, synthetic construct
  source 1..23: mol_type = other DNA
ggagcaacaaatctgactttgca

SEQ ID NO: 376 - DNA, 23 bp, synthetic construct
  source 1..23: mol_type = other DNA
aatgtgtcacaaagtaaggattc

SEQ ID NO: 377 - DNA, 23 bp, synthetic construct
  source 1..23: mol_type = other DNA
tctgatgtgtatatcacagacaa

SEQ ID NO: 378 - DNA, 24 bp, synthetic construct
  source 1..24: mol_type = other DNA
gcctggagcaacaaatctgacttt

SEQ ID NO: 379 - DNA, 23 bp, synthetic construct
  source 1..23: mol_type = other DNA
ggcgtttgcacatgcaaagtcag

SEQ ID NO: 380 - DNA, 23 bp, synthetic construct
  source 1..23: mol_type = other DNA
gaagaaggtgtcttctggaataa

SEQ ID NO: 381 - DNA, 24 bp, synthetic construct
  source 1..24: mol_type = other DNA
gctgcccttacctgggctggggaa

SEQ ID NO: 382 - DNA, 23 bp, synthetic construct
  source 1..23: mol_type = other DNA
tgggctggcggccaggatggttc

SEQ ID NO: 383 - DNA, 23 bp, synthetic construct
  source 1..23: mol_type = other DNA
ccgcccagacgactggccagggc

SEQ ID NO: 384 - DNA, 23 bp, synthetic construct
  source 1..23: mol_type = other DNA
caggcgccctggccagtcgtctg

SEQ ID NO: 385 - DNA, 23 bp, synthetic construct
  source 1..23: mol_type = other DNA
agaaccatcctggccgccagccc

SEQ ID NO: 386 - DNA, 23 bp, synthetic construct
  source 1..23: mol_type = other DNA
ctaagaaccatcctggccgccag

SEQ ID NO: 387 - DNA, 24 bp, synthetic construct
  source 1..24: mol_type = other DNA
gctggcggccaggatggttcttag

SEQ ID NO: 388 - DNA, 23 bp, synthetic construct
  source 1..23: mol_type = other DNA
acaactgggctggcggccaggat

SEQ ID NO: 389 - DNA, 23 bp, synthetic construct
  source 1..23: mol_type = other DNA
gtggggctgctccaggcatgcag

SEQ ID NO: 390 - DNA, 23 bp, synthetic construct
  source 1..23: mol_type = other DNA
ggccgccagcccagttgtagcac

SEQ ID NO: 391 - DNA, 28 bp, synthetic construct
  source 1..28: mol_type = other DNA
caccgcagaaagagagagtagcgcgagc

SEQ ID NO: 392 - DNA, 28 bp, synthetic construct
  source 1..28: mol_type = other DNA
caccgcacgtcatccagcagagaatgga

SEQ ID NO: 393 - DNA, 28 bp, synthetic construct
  source 1..28: mol_type = other DNA
caccgcattcttcagtaagtcaacttca

SEQ ID NO: 394 - DNA, 28 bp, synthetic construct
  source 1..28: mol_type = other DNA
caccgagcattcgggccgagatgtctcg

SEQ ID NO: 395 - DNA, 27 bp, synthetic construct
  source 1..27: mol_type = other DNA
caccgagatgtctcgctccgtggcctt

SEQ ID NO: 396 - DNA, 27 bp, synthetic construct
  source 1..27: mol_type = other DNA
caccggatagcctccaggccagaaaga

SEQ ID NO: 397 - DNA, 28 bp, synthetic construct
  source 1..28: mol_type = other DNA
caccgttgactttccattctctgctgga

SEQ ID NO: 398 - DNA, 27 bp, synthetic construct
  source 1..27: mol_type = other DNA
caccggaaagtcaaatttcctgaattg

SEQ ID NO: 399 - DNA, 28 bp, synthetic construct
  source 1..28: mol_type = other DNA
caccgtttgtctgtgatatacacatcag

SEQ ID NO: 400 - DNA, 28 bp, synthetic construct
  source 1..28: mol_type = other DNA
caccgtaaggattctgatgtgtatatca

SEQ ID NO: 401 - DNA, 27 bp, synthetic construct
  source 1..27: mol_type = other DNA
caccggccacagcactgttgctcttga

SEQ ID NO: 402 - DNA, 27 bp, synthetic construct
  source 1..27: mol_type = other DNA
caccgagcaacagtgctgtggcctgga

SEQ ID NO: 403 - DNA, 27 bp, synthetic construct
  source 1..27: mol_type = other DNA
caccggagcaacaaatctgactttgca

SEQ ID NO: 404 - DNA, 28 bp, synthetic construct
  source 1..28: mol_type = other DNA
caccgaatgtgtcacaaagtaaggattc

SEQ ID NO: 405 - DNA, 28 bp, synthetic construct
  source 1..28: mol_type = other DNA
caccgtctgatgtgtatatcacagacaa

SEQ ID NO: 406 - DNA, 28 bp, synthetic construct
  source 1..28: mol_type = other DNA
caccgcctggagcaacaaatctgacttt

SEQ ID NO: 407 - DNA, 27 bp, synthetic construct
  source 1..27: mol_type = other DNA
caccggcgtttgcacatgcaaagtcag

SEQ ID NO: 408 - DNA, 27 bp, synthetic construct
  source 1..27: mol_type = other DNA
caccgaagaaggtgtcttctggaataa

SEQ ID NO: 409 - DNA, 28 bp, synthetic construct
  source 1..28: mol_type = other DNA
caccgctgcccttacctgggctggggaa

SEQ ID NO: 410 - DNA, 28 bp, synthetic construct
  source 1..28: mol_type = other DNA
caccgtgggctggcggccaggatggttc

SEQ ID NO: 411 - DNA, 28 bp, synthetic construct
  source 1..28: mol_type = other DNA
caccgccgcccagacgactggccagggc

SEQ ID NO: 412 - DNA, 28 bp, synthetic construct
  source 1..28: mol_type = other DNA
caccgcaggcgccctggccagtcgtctg

SEQ ID NO: 413 - DNA, 28 bp, synthetic construct
  source 1..28: mol_type = other DNA
caccgagaaccatcctggccgccagccc

SEQ ID NO: 414 - DNA, 28 bp, synthetic construct
  source 1..28: mol_type = other DNA
caccgctaagaaccatcctggccgccag

SEQ ID NO: 415 - DNA, 28 bp, synthetic construct
  source 1..28: mol_type = other DNA
caccgctggcggccaggatggttcttag

SEQ ID NO: 416 - DNA, 28 bp, synthetic construct
  source 1..28: mol_type = other DNA
caccgacaactgggctggcggccaggat

SEQ ID NO: 417 - DNA, 27 bp, synthetic construct
  source 1..27: mol_type = other DNA
caccgtggggctgctccaggcatgcag

SEQ ID NO: 418 - DNA, 27 bp, synthetic construct
  source 1..27: mol_type = other DNA
caccggccgccagcccagttgtagcac

SEQ ID NO: 419 - DNA, 28 bp, synthetic construct
  source 1..28: mol_type = other DNA
agacgctcgcgctactctctctttctgc

SEQ ID NO: 420 - DNA, 28 bp, synthetic construct
  source 1..28: mol_type = other DNA
agactccattctctgctggatgacgtgc

SEQ ID NO: 421 - DNA, 28 bp, synthetic construct
  source 1..28: mol_type = other DNA
agactgaagttgacttactgaagaatgc

SEQ ID NO: 422 - DNA, 28 bp, synthetic construct
  source 1..28: mol_type = other DNA
agaccgagacatctcggcccgaatgctc

SEQ ID NO: 423 - DNA, 27 bp, synthetic construct
  source 1..27: mol_type = other DNA
agacaaggccacggagcgagacatctc

SEQ ID NO: 424 - DNA, 27 bp, synthetic construct
  source 1..27: mol_type = other DNA
agactctttctggcctggaggctatcc

SEQ ID NO: 425 - DNA, 28 bp, synthetic construct
  source 1..28: mol_type = other DNA
agactccagcagagaatggaaagtcaac

SEQ ID NO: 426 - DNA, 27 bp, synthetic construct
  source 1..27: mol_type = other DNA
agaccaattcaggaaatttgactttcc

SEQ ID NO: 427 - DNA, 28 bp, synthetic construct
  source 1..28: mol_type = other DNA
agacctgatgtgtatatcacagacaaac

SEQ ID NO: 428 - DNA, 28 bp, synthetic construct
  source 1..28: mol_type = other DNA
agactgatatacacatcagaatccttac

SEQ ID NO: 429 - DNA, 27 bp, synthetic construct
  source 1..27: mol_type = other DNA
agactcaagagcaacagtgctgtggcc

SEQ ID NO: 430 - DNA, 27 bp, synthetic construct
  source 1..27: mol_type = other DNA
agactccaggccacagcactgttgctc

SEQ ID NO: 431 - DNA, 27 bp, synthetic construct
  source 1..27: mol_type = other DNA
agactgcaaagtcagatttgttgctcc

SEQ ID NO: 432 - DNA, 28 bp, synthetic construct
  source 1..28: mol_type = other DNA
agacgaatccttactttgtgacacattc

SEQ ID NO: 433 - DNA, 28 bp, synthetic construct
  source 1..28: mol_type = other DNA
agacttgtctgtgatatacacatcagac

SEQ ID NO: 434 - DNA, 28 bp, synthetic construct
  source 1..28: mol_type = other DNA
agacaaagtcagatttgttgctccaggc

SEQ ID NO: 435 - DNA, 27 bp, synthetic construct
  source 1..27: mol_type = other DNA
agacctgactttgcatgtgcaaacgcc

SEQ ID NO: 436 - DNA, 27 bp, synthetic construct
  source 1..27: mol_type = other DNA
agacttattccagaagacaccttcttc

SEQ ID NO: 437 - DNA, 28 bp, synthetic construct
  source 1..28: mol_type = other DNA
agacttccccagcccaggtaagggcagc

SEQ ID NO: 438 - DNA, 28 bp, synthetic construct
  source 1..28: mol_type = other DNA
agacgaaccatcctggccgccagcccac

SEQ ID NO: 439 - DNA, 28 bp, synthetic construct
  source 1..28: mol_type = other DNA
agacgccctggccagtcgtctgggcggc

SEQ ID NO: 440 - DNA, 28 bp, synthetic construct
  source 1..28: mol_type = other DNA
agaccagacgactggccagggcgcctgc

SEQ ID NO: 441 - DNA, 28 bp, synthetic construct
  source 1..28: mol_type = other DNA
agacgggctggcggccaggatggttctc

SEQ ID NO: 442 - DNA, 28 bp, synthetic construct
  source 1..28: mol_type = other DNA
agacctggcggccaggatggttcttagc

SEQ ID NO: 443 - DNA, 28 bp, synthetic construct
  source 1..28: mol_type = other DNA
agacctaagaaccatcctggccgccagc

SEQ ID NO: 444 - DNA, 28 bp, synthetic construct
  source 1..28: mol_type = other DNA
agacatcctggccgccagcccagttgtc

SEQ ID NO: 445 - DNA, 27 bp, synthetic construct
  source 1..27: mol_type = other DNA
agacctgcatgcctggagcagccccac

SEQ ID NO: 446 - DNA, 27 bp, synthetic construct
  source 1..27: mol_type = other DNA
agacgtgctacaactgggctggcggcc

SEQ ID NO: 447 - DNA, 20 bp, synthetic construct
  source 1..20: mol_type = other DNA
catcacgagcagctggtttc

SEQ ID NO: 448 - DNA, 20 bp, synthetic construct
  source 1..20: mol_type = other DNA
ctctaacctggcactgcgtc

SEQ ID NO: 449 - DNA, 20 bp, synthetic construct
  source 1..20: mol_type = other DNA
tggccagagtggaaatggaa

SEQ ID NO: 450 - DNA, 20 bp, synthetic construct
  source 1..20: mol_type = other DNA
cactgcctctgtcactctcg

SEQ ID NO: 451 - DNA, 20 bp, synthetic construct
  source 1..20: mol_type = other DNA
tggcaatggataaggccgag

SEQ ID NO: 452 - DNA, 20 bp, synthetic construct
  source 1..20: mol_type = other DNA
cgaagtccacagctctccag

SEQ ID NO: 453 - DNA, 21 bp, synthetic construct
  source 1..21: mol_type = other DNA
tgtatttgtgcaagtgctgct

SEQ ID NO: 454 - DNA, 20 bp, synthetic construct
  source 1..20: mol_type = other DNA
gacttgggccaggggaggag

SEQ ID NO: 455 - DNA, 111 bp, unidentified
  source 1..111: mol_type = unassigned DNA
ctggagccctgagtggctgagctcaggccttcgcagcattcttgggtgggagcagccacgggtcagccacaagggccacagccatgaatggcacagaaggccctaacttct